(19) United States
(12) Patent Application Publication
(10) Pub. No.: US 2022/0169659 A1
(43) Pub. Date: Jun. 2, 2022

(54) HETEROCYCLIC COMPOUND AND ORGANIC LIGHT-EMITTING DEVICE COMPRISING SAME

(71) Applicant: LT MATERIALS CO., LTD., Yongin-si, Gyeonggi-do (KR)

(72) Inventors: Nam-Jin LEE, Yongin-si (KR); Yu-Jin HEO, Yongin-si (KR); Won-Jang JEONG, Yongin-si (KR); Dong-Jun KIM, Yongin-si (KR)

(73) Assignee: LT MATERIALS CO., LTD., Yongin-si, Gyeonggi-do (KR)

(21) Appl. No.: 17/441,390

(22) PCT Filed: Sep. 1, 2020
(86) PCT No.: PCT/KR2020/011674

§ 371 Date: Sep. 21, 2021

(30) Foreign Application Priority Data
Sep. 3, 2019 (KR) 10-2019-0108843

(51) Int. Cl. C07D 498/04; C07D 513/04; C07D 519/00; C07F 9/6561; H01L 51/00

(52) U.S. Cl. CPC C07D 498/04; C07D 513/04; C07D 519/00; C07F 9/6561; H01L 51/0067; H01L 51/5056; H01L 51/0072; H01L 51/0056; H01L 51/006; H01L 51/0054; H01L 51/0073; H01L 51/0058

(57) ABSTRACT

The present specification relates to a heterocyclic compound represented by Chemical Formula 1, and an organic light emitting device comprising the same.

### TECHNICAL FIELD

This application claims priority to and the benefits of Korean Patent Application No. 10-2019-0108843, filed with the Korean Intellectual Property Office on Sep. 3, 2019, the entire contents of which are incorporated herein by reference.

The present specification relates to a heterocyclic compound, and an organic light emitting device comprising the same.

### BACKGROUND ART

An organic electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

U.S. Pat. No. 4,356,429

### DISCLOSURE

### Technical Problem

The present disclosure is directed to providing a heterocyclic compound, and an organic light emitting device comprising the same.

### Technical Solution

One embodiment of the present application provides a heterocyclic compound represented by the following Chemical Formula 1.

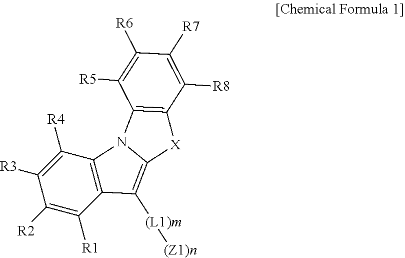

In Chemical Formula 1,

X is O; S; or NRa,

R1 to R8 and Ra are the same as or different from each other, and each independently selected from the group consisting of hydrogen; deuterium; a halogen group; —CN; a substituted or unsubstituted C1 to C60 alkyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR11R12R13; —P(═O)R14R15; and —NR16R17, or two or more groups adjacent to each other bond to each other to form a substituted or unsubstituted C6 to C60 aromatic hydrocarbon ring or a substituted or unsubstituted C2 to C60 heteroring,

L1 is a direct bond; a substituted or unsubstituted C6 to C60 arylene group; a substituted or unsubstituted C2 to C60 heteroarylene group; or a substituted or unsubstituted divalent amine group,

Z1 is a halogen group; —CN; a substituted or unsubstituted C1 to C60 alkyl group; a substituted or unsubstituted C6 to C60 aryl group; a substituted or unsubstituted C2 to C60 heteroaryl group; —SiR11R12R13; —P(═O)R14R15; or —NR16R17,

R11 to R17 are the same as or different from each other, and each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group,

m is an integer of 0 to 4, and when m is 2 or greater, two or more L1s are the same as or different from each other, and

n is an integer of 1 to 5, and when n is 2 or greater, two or more Z1s are the same as or different from each other.

Another embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

### Advantageous Effects

A compound described in the present specification can be used as a material of an organic material layer of an organic light emitting device. In the organic light emitting device, the compound is capable of performing a role of a hole injection material, a hole transfer material, a hole blocking material, a light emitting material, an electron transfer material, an electron injection material, a charge generation material or the like. Particularly, the compound can be used as an electron transfer layer material, a hole transfer layer material or a charge generation layer material of an organic light emitting device.

When using the compound represented by Chemical Formula 1 in an organic material layer, a driving voltage of a device can be lowered, light efficiency can be enhanced, and lifetime properties of the device can be enhanced by thermal stability of the compound.

The compound represented by Chemical Formula 1 has a core form of fused four rings having a heteroatom, and by having a more electron-friendly heteroatom added to the central skeleton of the core structure and thereby having an enhanced electron transfer ability, superior device properties are obtained when used in an organic light emitting device afterward.

### DESCRIPTION OF DRAWINGS

FIG. 1 to FIG. 4 are diagrams each schematically illustrating a lamination structure of an organic light emitting device according to one embodiment of the present application.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

### MODE FOR DISCLOSURE

Hereinafter, the present application will be described in detail.

In the present specification, the term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, and

R, R′ and R″ are the same as or different from each other, and each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0% or a hydrogen content being 100%. In other words, an expression of “substituent X is hydrogen” does not exclude deuterium such as a hydrogen content being 100% or a deuterium content being 0%, and therefore, may mean a state in which hydrogen and deuterium are mixed.

In one embodiment of the present application, deuterium is one of isotopes of hydrogen, is an element having deuteron formed with one proton and one neutron as a nucleus, and may be expressed as hydrogen-2, and the elemental symbol may also be written as D or 2H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

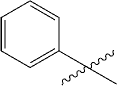 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

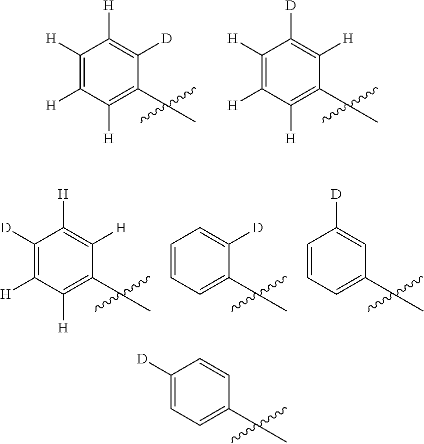 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present application, “a phenyl group having a deuterium content of 0%” may mean a phenyl group that does not comprise a deuterium atom, that is, a phenyl group that has 5 hydrogen atoms.

In the present specification, the halogen may be fluorine, chlorine, bromine or iodine.

In the present specification, the alkyl group comprises linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group comprises linear or branched having 2 to 60 carbon atoms, and may be further substituted with other substituents. The number of carbon atoms of the alkenyl group may be from 2 to 60, specifically from 2 to 40 and more specifically from 2 to 20.

Specific examples thereof may comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group comprises linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may comprise methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group comprises monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group comprises O, S, Se, N or Si as a heteroatom, comprises monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group comprises monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group comprises a spiro group. The number of carbon atoms of the aryl group may be from 6 to 60, specifically from 6 to 40 and more specifically from 6 to 25. Specific examples of the aryl group may comprise a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the phosphine oxide may comprise a diphenylphosphine oxide group, a dinaphthylphosphine oxide group and the like, but are not limited thereto.

In the present specification, the silyl group is a substituent comprising Si, having the Si atom directly linked as a radical, and is represented by —SiR₁₀₄R₁₀₅R₁₀₆. R₁₀₄ to R₁₀₆ are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted, the following structures may be included, however, the structure is not limited thereto.

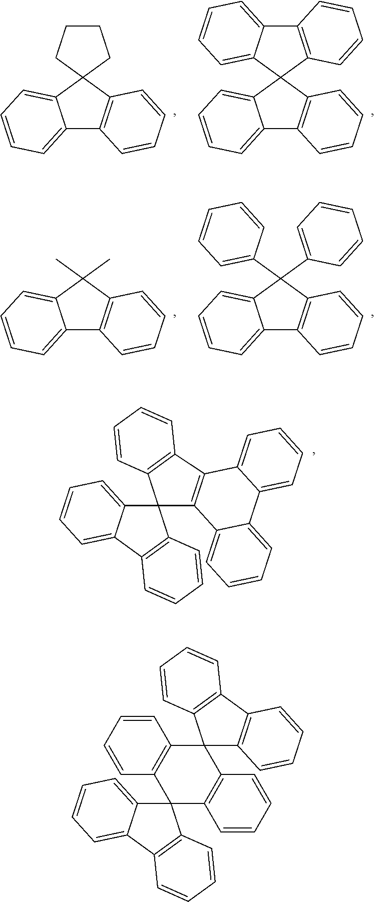

In the present specification, the heteroaryl group comprises S, O, Se, N or Si as a heteroatom, comprises monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

One embodiment of the present application provides a compound represented by Chemical Formula 1.

In one embodiment of the present application, X may be 0.

In one embodiment of the present application, X may be S.

In one embodiment of the present application, X may be NRa.

In one embodiment of the present application, L1 may be a direct bond; a substituted or unsubstituted C6 to C60 arylene group; a substituted or unsubstituted C2 to C60 heteroarylene group; or a substituted or unsubstituted divalent amine group.

In another embodiment, L1 may be a direct bond; a substituted or unsubstituted C6 to C40 arylene group; a substituted or unsubstituted C2 to C40 heteroarylene group; or a substituted or unsubstituted divalent amine group.

In another embodiment, L1 may be a direct bond; a C6 to C40 arylene group unsubstituted or substituted with a C2 to C40 heteroaryl group; a C2 to C40 heteroarylene group unsubstituted or substituted with a C6 to C40 aryl group; or a divalent amine group unsubstituted or substituted with a C6 to C40 aryl group.

In another embodiment, L1 may be a direct bond; a C6 to C40 monocyclic or polycyclic arylene group unsubstituted or substituted with a C2 to C40 heteroaryl group; a C2 to C40 monocyclic or polycyclic heteroarylene group unsubstituted or substituted with a C6 to C40 aryl group; or a divalent amine group unsubstituted or substituted with a C6 to C40 aryl group.

In another embodiment, L1 may be a direct bond; a C6 to C40 monocyclic arylene group unsubstituted or substituted with a C2 to C40 heteroaryl group; a C10 to C40 polycyclic arylene group unsubstituted or substituted with a C2 to C40 heteroaryl group; a C2 to C40 monocyclic heteroarylene group unsubstituted or substituted with a C6 to C40 aryl group; a C2 to C40 polycyclic heteroarylene group unsubstituted or substituted with a C6 to C40 aryl group; or a divalent amine group unsubstituted or substituted with a C6 to C40 aryl group.

In another embodiment, L1 may be a direct bond; a phenylene group unsubstituted or substituted with a carbazole group; a biphenylene group; a naphthalene group; a triazine group unsubstituted or substituted with a phenyl group; or a divalent amine group unsubstituted or substituted with a phenyl group.

In another embodiment, L1 may be a direct bond; a phenylene group unsubstituted or substituted with a carbazole group; a biphenylene group; a naphthalene group; a triazine group unsubstituted or substituted with a phenyl group; or a divalent phenylamine group.

In one embodiment of the present application, Z1 may be a halogen group; —CN; a substituted or unsubstituted C1 to C60 alkyl group; a substituted or unsubstituted C6 to C60 aryl group; a substituted or unsubstituted C2 to C60 heteroaryl group; —SiR11R12R13; —P(═O)R14R15; or —NR16R17.

In another embodiment, Z1 may be a halogen group; —CN; a substituted or unsubstituted C1 to C40 alkyl group; a substituted or unsubstituted C6 to C40 aryl group; a substituted or unsubstituted C2 to C40 heteroaryl group; —SiR11R12R13; —P(═O)R14R15; or —NR16R17.

In another embodiment, Z1 may be a C1 to C40 alkyl group; a C6 to C40 aryl group; a C2 to C40 heteroaryl group unsubstituted or substituted with one or more substituents selected from the group consisting of a C1 to C20 alkyl group, a C6 to C40 aryl group and a C2 to C40 heteroaryl group; —P(═O)R14R15; or —NR16R17.

In another embodiment, Z1 may be a methyl group; an ethyl group; a phenyl group; a biphenyl group; a naphthylenyl group; a triphenylenyl group; a triazine group unsubstituted or substituted with one or more substituents selected from the group consisting of a phenyl group, a biphenyl group and a naphthylenyl group; a pyrimidine group unsubstituted or substituted with one or more substituents selected from the group consisting of a phenyl group, a biphenyl group and a naphthylenyl group; a pyridine group unsubstituted or substituted with a pyridine group; a quinazoline group unsubstituted or substituted with a phenyl group; a phenanthroline group unsubstituted or substituted with a phenyl group; a benzimidazole group unsubstituted or substituted with a phenyl group or an ethyl group; a dibenzofuran group; a carbazole group; —P(═O)R14R15; or —NR16R17.

In one embodiment of the present application, Z1 may be substituted again with a C2 to C40 heteroaryl group; or a C1 to C20 alkyl group.

In another embodiment, Z1 may be substituted again with a methyl group; a carbazole group; or a dibenzofuran group.

In one embodiment of the present application, R11 to R17 are the same as or different from each other, and may be each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group.

In another embodiment, R11 to R17 are the same as or different from each other, and may be each independently hydrogen; a halogen group; —CN; a substituted or unsubstituted C1 to C40 alkyl group; a substituted or unsubstituted C6 to C40 aryl group; or a substituted or unsubstituted C2 to C40 heteroaryl group.

In another embodiment, R11 to R17 are the same as or different from each other, and may be each independently a C6 to C40 aryl group unsubstituted or substituted with a C1 to C20 alkyl group or a C6 to C40 aryl group; or a C2 to C40 heteroaryl group unsubstituted or substituted with a C1 to C20 alkyl group or a C6 to C40 aryl group.

In another embodiment, R11 to R17 are the same as or different from each other, and may be each independently a phenyl group; a naphthylenyl group; a biphenyl group; a dimethylfluorenyl group; a diphenylfluorenyl group; a spirobifluorenyl group; or a dibenzofuran group.

In one embodiment of the present application, R14 and R15 may be a phenyl group; or a naphthylenyl group.

In one embodiment of the present application, R16 and R17 may be a phenyl group; a biphenyl group; a naphthylenyl group; a dimethylfluorenyl group; a diphenylfluorenyl group; a spirobifluorenyl group; or a dibenzofuran group.

In one embodiment of the present application, Chemical Formula 1 may be represented by any one of the following Chemical Formula 2 to Chemical Formula 4.

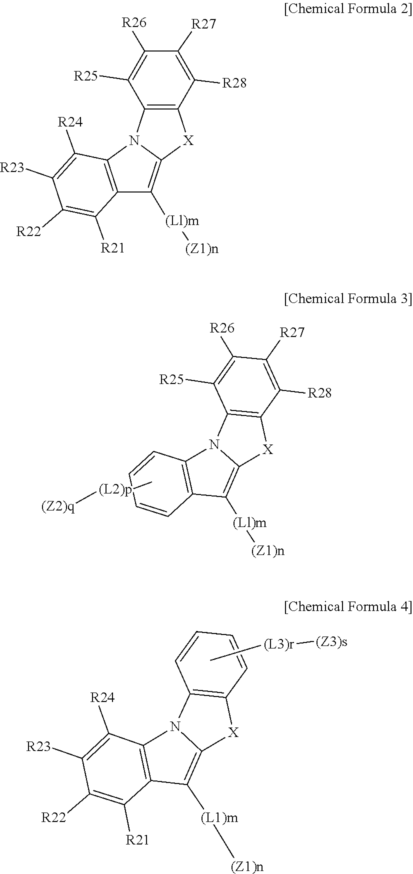

In Chemical Formulae 2 to 4,

X, m, n, L1 and Z1 have the same definitions as in Chemical Formula 1,

R21 to R28 are the same as or different from each other, and are hydrogen; or deuterium,

L2 and L3 are the same as or different from each other, and each independently a direct bond; a substituted or unsubstituted C6 to C60 arylene group; or a substituted or unsubstituted C2 to C60 heteroarylene group,

Z2 and Z3 are the same as or different from each other, and each independently a halogen group; —CN; a substituted or unsubstituted C1 to C60 alkyl group; a substituted or unsubstituted C6 to C60 aryl group; a substituted or unsubstituted C2 to C60 heteroaryl group; —SiR31R32R33; —P(═O)R34R35; or —NR36R37,

R31 to R37 are the same as or different from each other, and each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group,

p and r are an integer of 0 to 4, and

q and s are an integer of 1 to 5.

In one embodiment of the present application, Chemical Formula 3 may be represented by any one of the following Chemical Formula 3-1 to Chemical Formula 3-4.

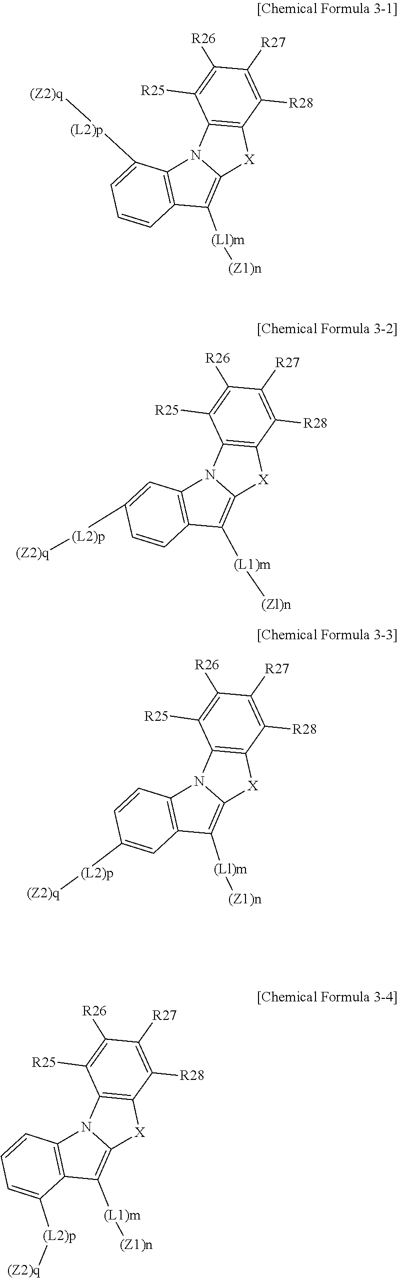

In Chemical Formulae 3-1 to 3-4,

X, L1, L2, Z1, Z2, m, n, p, q and R25 to R28 have the same definitions as in Chemical Formula 3.

In one embodiment of the present application, Chemical Formula 4 may be represented by any one of the following Chemical Formula 4-1 to Chemical Formula 4-4.

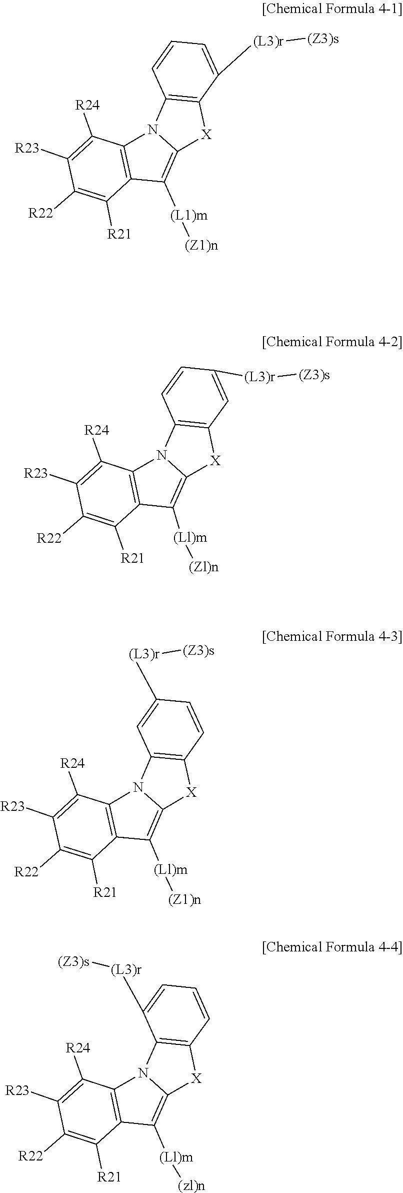

In Chemical Formulae 4-1 to 4-4,

X, L1, L3, Z1, Z3, m, n, r, s and R21 to R24 have the same definitions as in Chemical Formula 4.

In one embodiment of the present application, L2 and L3 are the same as or different from each other, and may be each independently a direct bond; a substituted or unsubstituted C6 to C60 arylene group; or a substituted or unsubstituted C2 to C60 heteroarylene group.

In another embodiment, L2 and L3 are the same as or different from each other, and may be each independently a direct bond; or a substituted or unsubstituted C6 to C60 arylene group.

In another embodiment, L2 and L3 are the same as or different from each other, and may be each independently a direct bond; or a substituted or unsubstituted C6 to C40 arylene group.

In another embodiment, L2 and L3 are the same as or different from each other, and may be each independently a direct bond; or a C6 to C40 monocyclic or polycyclic arylene group.

In another embodiment, L2 and L3 are the same as or different from each other, and may be each independently a direct bond; or a C6 to C20 monocyclic arylene group.

In another embodiment, L2 and L3 are the same as or different from each other, and may be each independently a direct bond; or a phenylene group.

In one embodiment of the present application, Z2 and Z3 are the same as or different from each other, and may be each independently a halogen group; —CN; a substituted or unsubstituted C1 to C60 alkyl group; a substituted or unsubstituted C6 to C60 aryl group; a substituted or unsubstituted C2 to C60 heteroaryl group; —SiR31R32R33; —P(═O)R34R35; or —NR36R37.

In another embodiment, Z2 and Z3 are the same as or different from each other, and may be each independently —CN; a substituted or unsubstituted C6 to C60 aryl group; a substituted or unsubstituted C2 to C60 heteroaryl group; or —NR36R37.

In another embodiment, Z2 and Z3 are the same as or different from each other, and may be each independently —CN; a substituted or unsubstituted C6 to C40 aryl group; a substituted or unsubstituted C2 to C40 heteroaryl group; or —NR36R37.

In another embodiment, Z2 and Z3 are the same as or different from each other, and may be each independently —CN; a C6 to C40 aryl group; a C2 to C40 heteroaryl group unsubstituted or substituted with a C6 to C40 aryl group; or —NR36R37.

In another embodiment, Z2 and Z3 are the same as or different from each other, and may be each independently —CN; a phenyl group; a naphthylenyl group; a biphenyl group; a triphenylenyl group; a dibenzofuran group; a pyrimidine group unsubstituted or substituted with a phenyl group; or —NR36R37.

In another embodiment, Z2 and Z3 are the same as or different from each other, and may be each independently —CN; a phenyl group; a naphthylenyl group; a biphenyl group; a triphenylenyl group; a dibenzofuran group; a pyrimidine group unsubstituted or substituted with a phenyl group; a phenylnaphthylamine group; a dinaphthylamine group; or a diphenylamine group.

In one embodiment of the present application, R31 to R37 are the same as or different from each other, and may be each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group.

In another embodiment, R31 to R37 are the same as or different from each other, and may be each independently a substituted or unsubstituted C6 to C60 aryl group.

In another embodiment, R31 to R37 are the same as or different from each other, and may be each independently a C6 to C60 aryl group.

In another embodiment, R31 to R37 are the same as or different from each other, and may be each independently a C6 to C40 monocyclic or polycyclic aryl group.

In another embodiment, R31 to R37 are the same as or different from each other, and may be each independently a C6 to C40 monocyclic aryl group; or a C10 to C40 polycyclic aryl group.

In another embodiment, R31 to R37 are the same as or different from each other, and may be each independently a phenyl group; or a naphthylenyl group.

In one embodiment of the present application, Chemical Formula 1 may be represented by any one of the following Chemical Formulae 5 to 7.

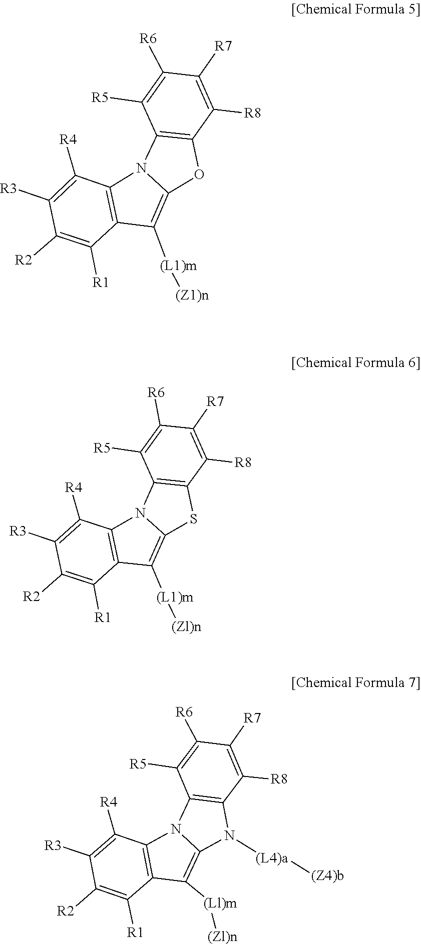

In Chemical Formulae 5 to 7,

R1 to R8, L1, Z1, m and n have the same definitions as in Chemical Formula 1,

L4 is a direct bond; a substituted or unsubstituted C6 to C60 arylene group; a substituted or unsubstituted C2 to C60 heteroarylene group; or a substituted or unsubstituted divalent amine group,

Z4 is a halogen group; —CN; a substituted or unsubstituted C1 to C60 alkyl group; a substituted or unsubstituted C6 to C60 aryl group; a substituted or unsubstituted C2 to C60 heteroaryl group; —SiR41R42R43; —P(═O)R44R45; or —NR46R47,

R41 to R47 are the same as or different from each other, and each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group,

a is an integer of 0 to 4, and when a is 2 or greater, two or more L4s are the same as or different from each other, and

b is an integer of 1 to 5, and when b is 2 or greater, two or more Z4s are the same as or different from each other.

In one embodiment of the present application, L4 has the same definition as L1.

In one embodiment of the present application, Z4 has the same definition as Z1.

In one embodiment of the present application, R1 to R8 are the same as or different from each other, and each independently selected from the group consisting of hydrogen; deuterium; a halogen group; —CN; a substituted or unsubstituted C1 to C60 alkyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR11R12R13; —P(═O)R14R15; and —NR16R17, or two or more groups adjacent to each other may bond to each other to form a substituted or unsubstituted C6 to C60 aromatic hydrocarbon ring or a substituted or unsubstituted C2 to C60 heteroring.

In another embodiment, R1 to R8 are the same as or different from each other, and each independently selected from the group consisting of hydrogen; —CN; a substituted or unsubstituted C1 to C60 alkyl group; a substituted or unsubstituted C6 to C60 aryl group; a substituted or unsubstituted C2 to C60 heteroaryl group; —P(═O)R14R15; and —NR16R17, or two or more groups adjacent to each other may bond to each other to form a substituted or unsubstituted C6 to C60 aromatic hydrocarbon ring or a substituted or unsubstituted C2 to C60 heteroring.

In another embodiment, R1 to R8 are the same as or different from each other, and may be each independently selected from the group consisting of hydrogen; —CN; a substituted or unsubstituted C1 to C60 alkyl group; a substituted or unsubstituted C6 to C60 aryl group; a substituted or unsubstituted C2 to C60 heteroaryl group; —P(═O)R14R15; and —NR16R17.

In another embodiment, R1 to R8 are the same as or different from each other, and may be each independently selected from the group consisting of hydrogen; —CN; a C1 to C60 alkyl group unsubstituted or substituted with one or more substituents selected from the group consisting of a C6 to C40 aryl group and a C2 to C40 heteroaryl group; a C6 to C60 aryl group unsubstituted or substituted with one or more substituents selected from the group consisting of a C1 to C20 alkyl group, a C6 to C40 aryl group and a C2 to C40 heteroaryl group; a C2 to C60 heteroaryl group unsubstituted or substituted with one or more substituents selected from the group consisting of a C1 to C20 alkyl group, a C6 to C40 aryl group and a C2 to C40 heteroaryl group; —P(═O)R14R15; and —NR16R17.

In the heterocyclic compound provided in one embodiment of the present application, Chemical Formula 1 is represented by any n of h following

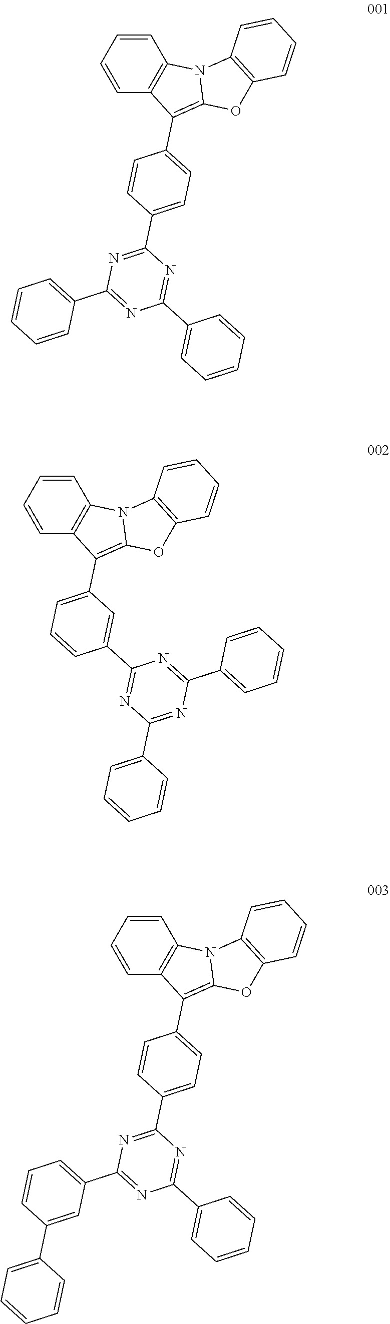 [SMILES: c1ccc(-c2nc(-c3ccccc3)nc(-c3ccc(-c4c5ccccc5n5c4oc4ccccc45)cc3)n2)cc1 ; c1ccc(-c2nc(-c3ccccc3)nc(-c3cccc(-c4c5ccccc5n5c4oc4ccccc45)c3)n2)cc1 ; c1ccc(-c2cccc(-c3nc(-c4ccccc4)nc(-c4ccc(-c5c6ccccc6n6c5oc5ccccc56)cc4)n3)c2)cc1]

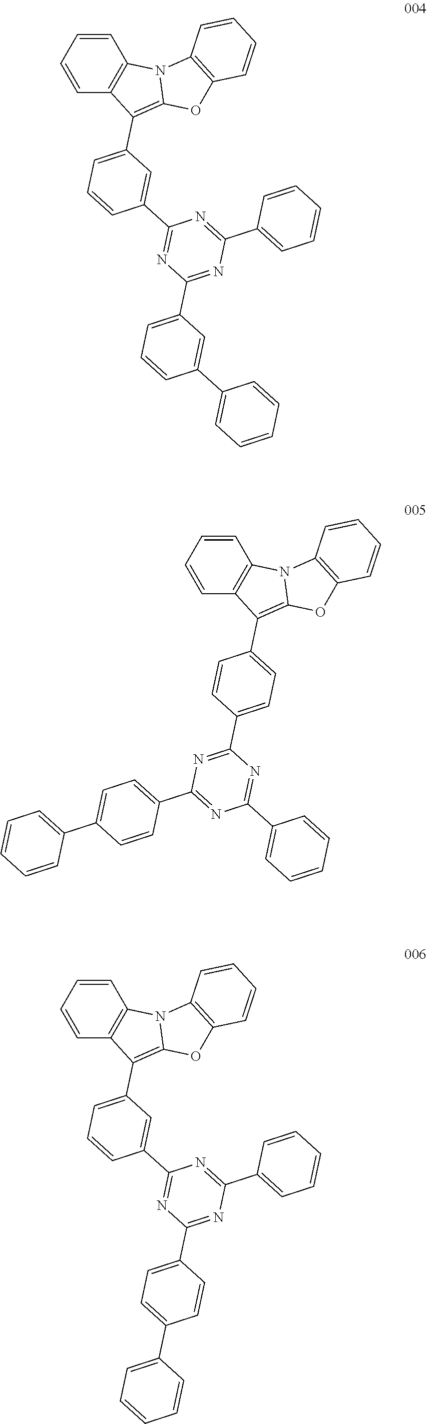 [SMILES: c1ccc(-c2cccc(-c3nc(-c4ccccc4)nc(-c4cccc(-c5c6ccccc6n6c5oc5ccccc56)c4)n3)c2)cc1 ; c1ccc(-c2ccc(-c3nc(-c4ccccc4)nc(-c4ccc(-c5c6ccccc6n6c5oc5ccccc56)cc4)n3)cc2)cc1 ; c1ccc(-c2ccc(-c3nc(-c4ccccc4)nc(-c4cccc(-c5c6ccccc6n6c5oc5ccccc56)c4)n3)cc2)cc1]

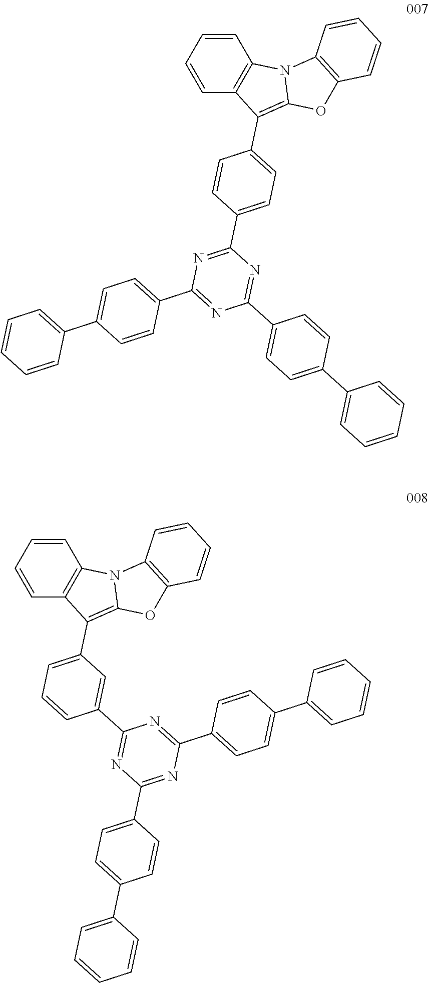 [SMILES: c1ccc(-c2ccc(-c3nc(-c4ccc(-c5ccccc5)cc4)nc(-c4ccc(-c5c6ccccc6n6c5oc5ccccc56)cc4)n3)cc2)cc1 ; c1ccc(-c2ccc(-c3nc(-c4ccc(-c5ccccc5)cc4)nc(-c4cccc(-c5c6ccccc6n6c5oc5ccccc56)c4)n3)cc2)cc1]

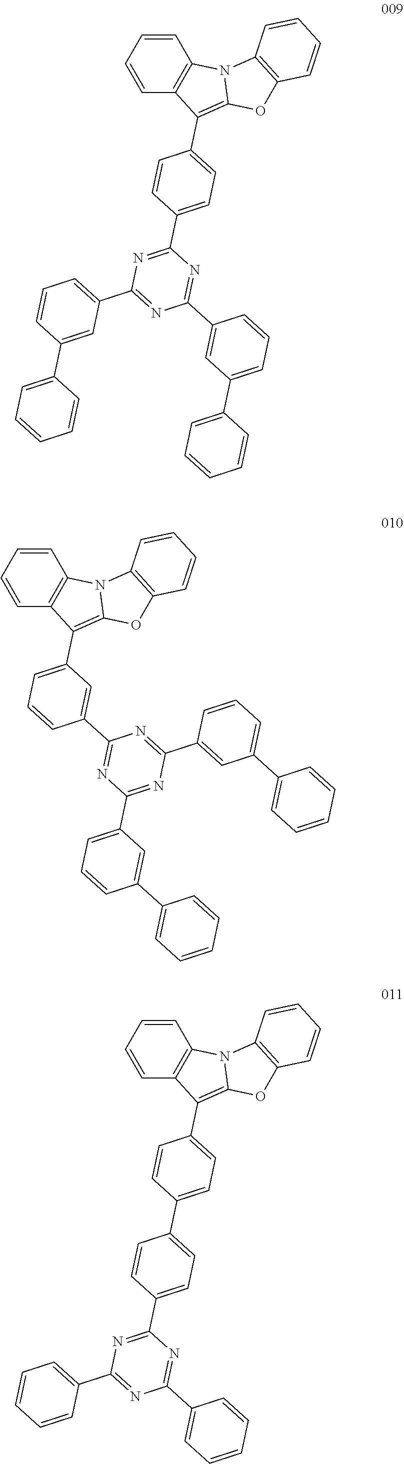 [SMILES: c1ccc(-c2cccc(-c3nc(-c4ccc(-c5c6ccccc6n6c5oc5ccccc56)cc4)nc(-c4cccc(-c5ccccc5)c4)n3)c2)cc1 ; c1ccc(-c2cccc(-c3nc(-c4cccc(-c5ccccc5)c4)nc(-c4cccc(-c5c6ccccc6n6c5oc5ccccc56)c4)n3)c2)cc1 ; c1ccc(-c2nc(-c3ccccc3)nc(-c3ccc(-c4ccc(-c5c6ccccc6n6c5oc5ccccc56)cc4)cc3)n2)cc1]

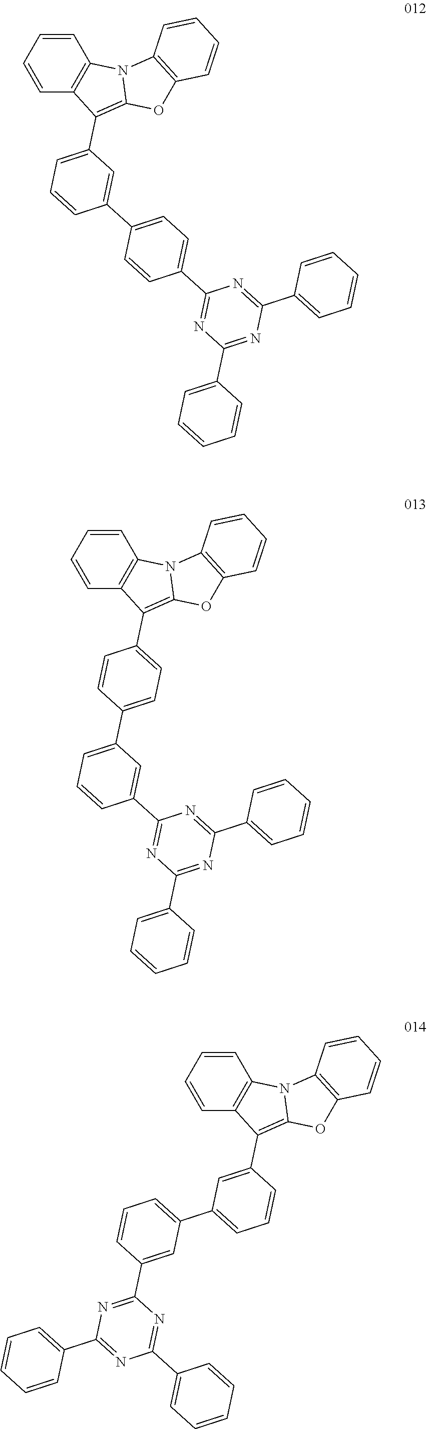 [SMILES: c1ccc(-c2nc(-c3ccccc3)nc(-c3ccc(-c4cccc(-c5c6ccccc6n6c5oc5ccccc56)c4)cc3)n2)cc1 ; c1ccc(-c2nc(-c3ccccc3)nc(-c3cccc(-c4ccc(-c5c6ccccc6n6c5oc5ccccc56)cc4)c3)n2)cc1 ; c1ccc(-c2nc(-c3ccccc3)nc(-c3cccc(-c4cccc(-c5c6ccccc6n6c5oc5ccccc56)c4)c3)n2)cc1]

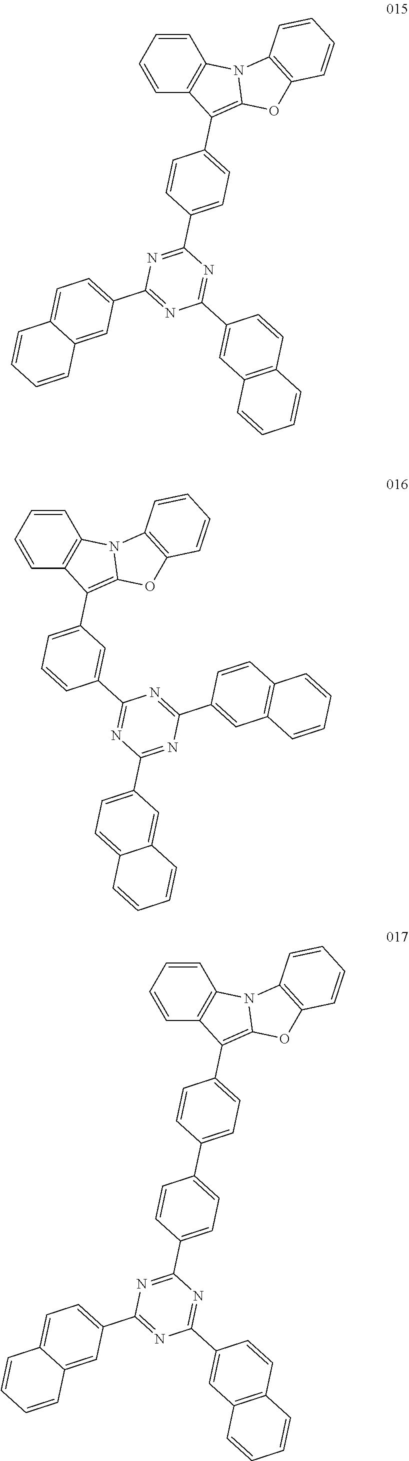 [SMILES: c1ccc2cc(-c3nc(-c4ccc(-c5c6ccccc6n6c5oc5ccccc56)cc4)nc(-c4ccc5ccccc5c4)n3)ccc2c1 ; c1cc(-c2nc(-c3ccc4ccccc4c3)nc(-c3ccc4ccccc4c3)n2)cc(-c2c3ccccc3n3c2oc2ccccc23)c1 ; c1ccc2cc(-c3nc(-c4ccc(-c5ccc(-c6c7ccccc7n7c6oc6ccccc67)cc5)cc4)nc(-c4ccc5ccccc5c4)n3)ccc2c1]

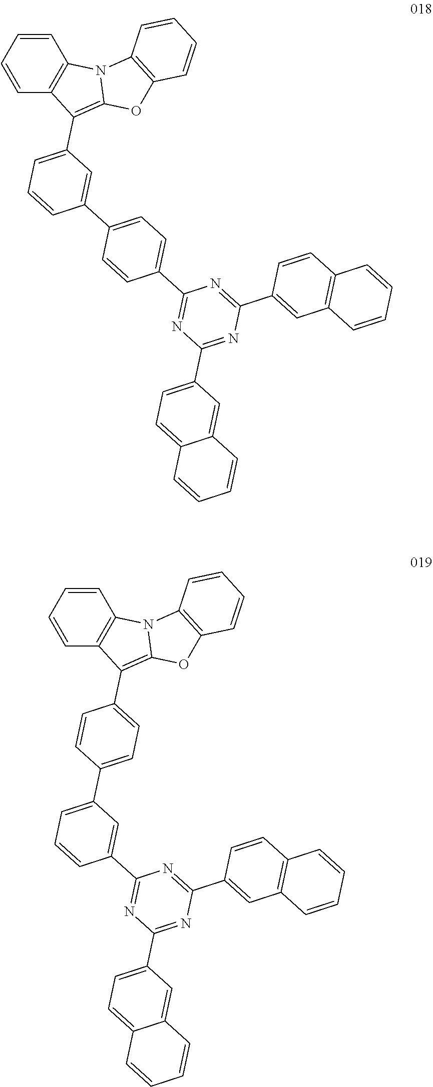 [SMILES: c1cc(-c2ccc(-c3nc(-c4ccc5ccccc5c4)nc(-c4ccc5ccccc5c4)n3)cc2)cc(-c2c3ccccc3n3c2oc2ccccc23)c1 ; c1cc(-c2ccc(-c3c4ccccc4n4c3oc3ccccc34)cc2)cc(-c2nc(-c3ccc4ccccc4c3)nc(-c3ccc4ccccc4c3)n2)c1]

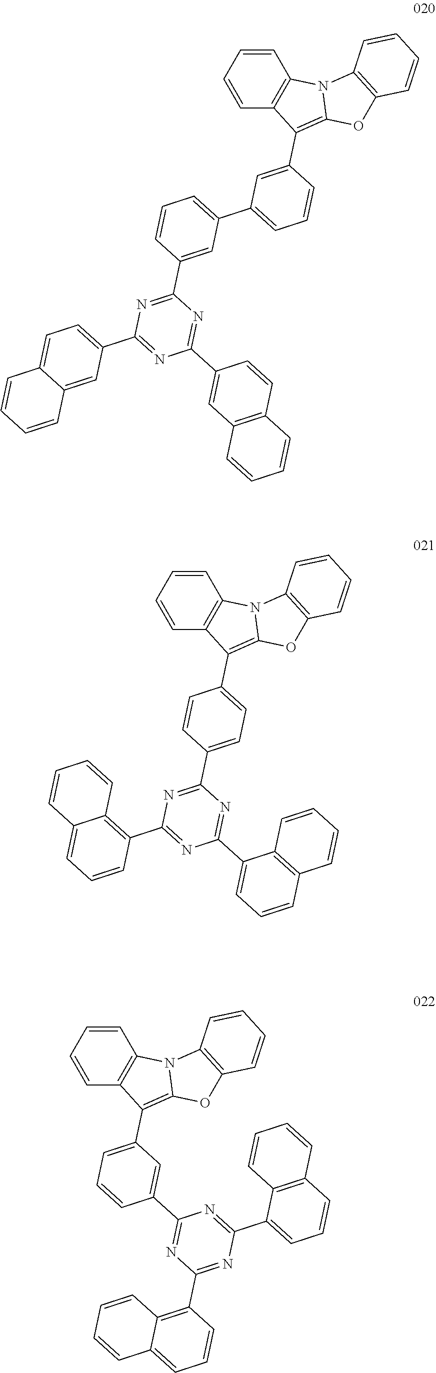 [SMILES: c1cc(-c2cccc(-c3c4ccccc4n4c3oc3ccccc34)c2)cc(-c2nc(-c3ccc4ccccc4c3)nc(-c3ccc4ccccc4c3)n2)c1 ; c1ccc2c(-c3nc(-c4ccc(-c5c6ccccc6n6c5oc5ccccc56)cc4)nc(-c4cccc5ccccc45)n3)cccc2c1 ; c1cc(-c2nc(-c3cccc4ccccc34)nc(-c3cccc4ccccc34)n2)cc(-c2c3ccccc3n3c2oc2ccccc23)c1]

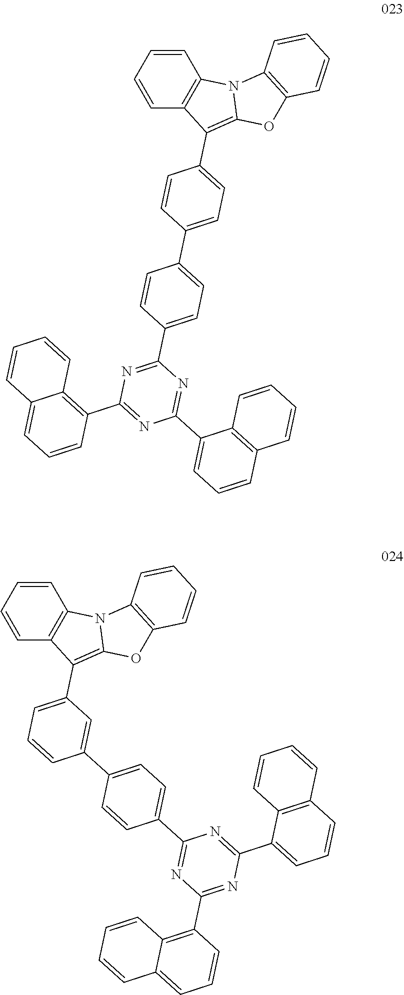 [SMILES: c1ccc2c(-c3nc(-c4ccc(-c5ccc(-c6c7ccccc7n7c6oc6ccccc67)cc5)cc4)nc(-c4cccc5ccccc45)n3)cccc2c1 ; c1cc(-c2ccc(-c3nc(-c4cccc5ccccc45)nc(-c4cccc5ccccc45)n3)cc2)cc(-c2c3ccccc3n3c2oc2ccccc23)c1]

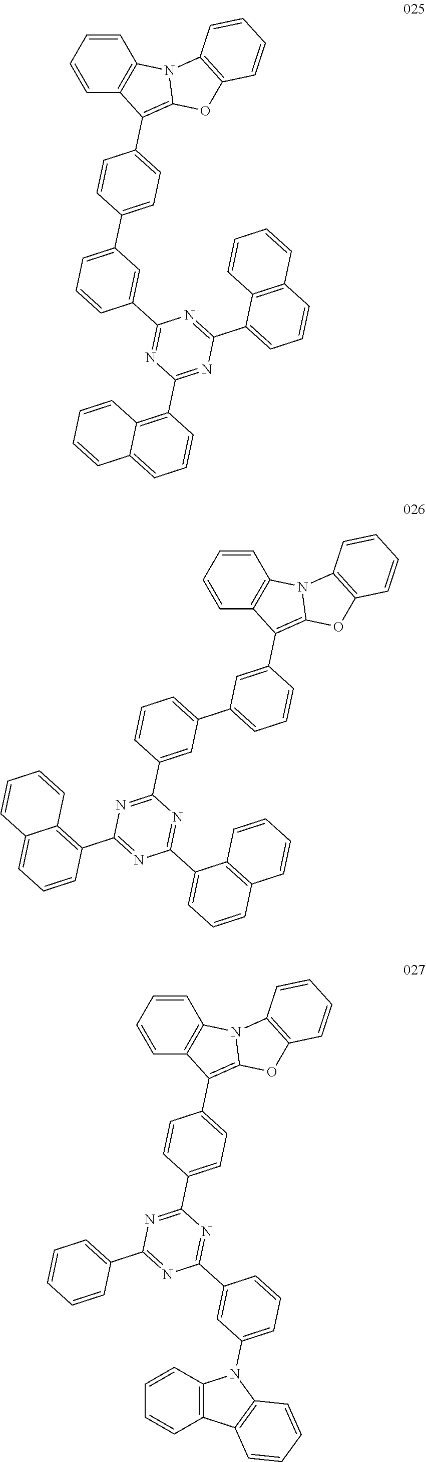 [SMILES: c1cc(-c2ccc(-c3c4ccccc4n4c3oc3ccccc34)cc2)cc(-c2nc(-c3cccc4ccccc34)nc(-c3cccc4ccccc34)n2)c1 ; c1cc(-c2cccc(-c3c4ccccc4n4c3oc3ccccc34)c2)cc(-c2nc(-c3cccc4ccccc34)nc(-c3cccc4ccccc34)n2)c1 ; c1ccc(-c2nc(-c3ccc(-c4c5ccccc5n5c4oc4ccccc45)cc3)nc(-c3cccc(-n4c5ccccc5c5ccccc54)c3)n2)cc1]

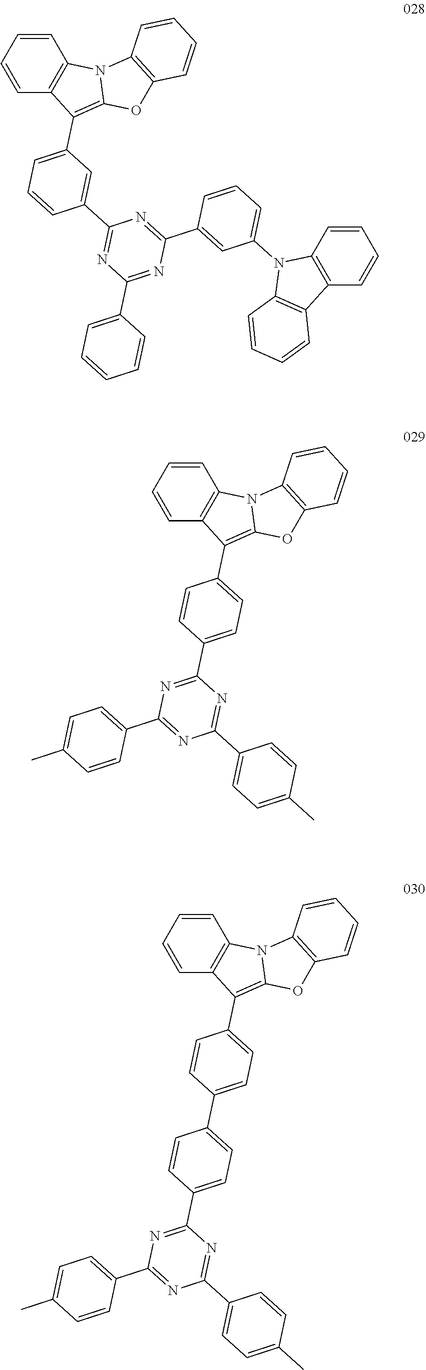 [SMILES: c1ccc(-c2nc(-c3cccc(-c4c5ccccc5n5c4oc4ccccc45)c3)nc(-c3cccc(-n4c5ccccc5c5ccccc54)c3)n2)cc1 ; Cc1ccc(-c2nc(-c3ccc(C)cc3)nc(-c3ccc(-c4c5ccccc5n5c4oc4ccccc45)cc3)n2)cc1 ; Cc1ccc(-c2nc(-c3ccc(C)cc3)nc(-c3ccc(-c4ccc(-c5c6ccccc6n6c5oc5ccccc56)cc4)cc3)n2)cc1]

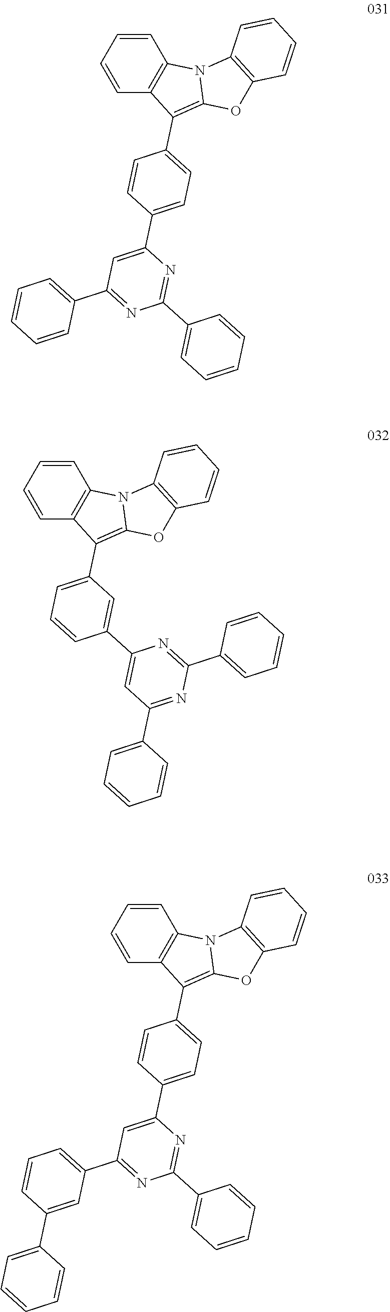 [SMILES: c1ccc(-c2cc(-c3ccc(-c4c5ccccc5n5c4oc4ccccc45)cc3)nc(-c3ccccc3)n2)cc1 ; c1ccc(-c2cc(-c3cccc(-c4c5ccccc5n5c4oc4ccccc45)c3)nc(-c3ccccc3)n2)cc1 ; c1ccc(-c2cccc(-c3cc(-c4ccc(-c5c6ccccc6n6c5oc5ccccc56)cc4)nc(-c4ccccc4)n3)c2)cc1]

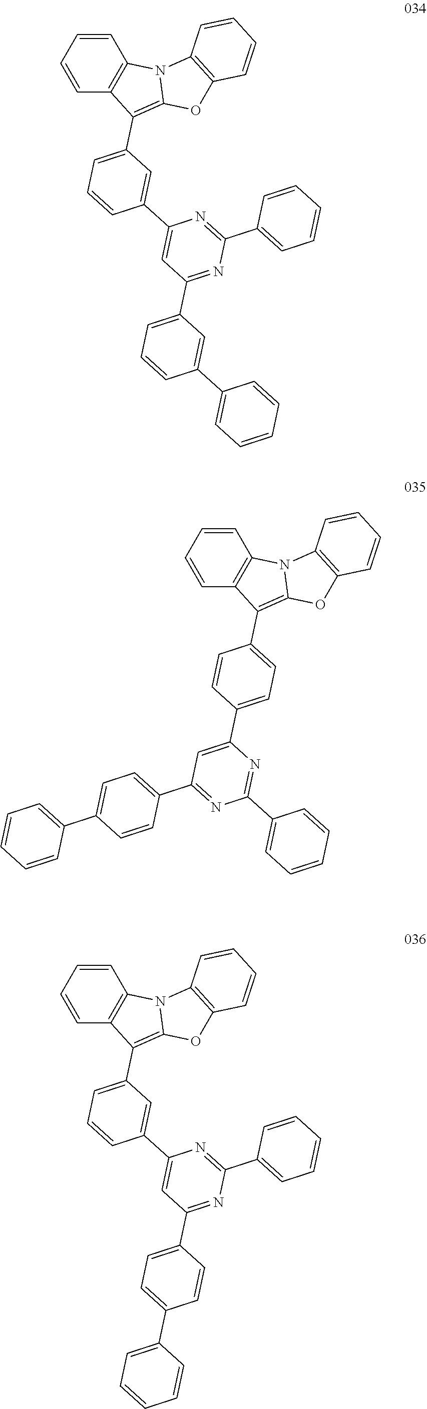 [SMILES: c1ccc(-c2cccc(-c3cc(-c4cccc(-c5c6ccccc6n6c5oc5ccccc56)c4)nc(-c4ccccc4)n3)c2)cc1 ; c1ccc(-c2ccc(-c3cc(-c4ccc(-c5c6ccccc6n6c5oc5ccccc56)cc4)nc(-c4ccccc4)n3)cc2)cc1 ; c1ccc(-c2ccc(-c3cc(-c4cccc(-c5c6ccccc6n6c5oc5ccccc56)c4)nc(-c4ccccc4)n3)cc2)cc1]

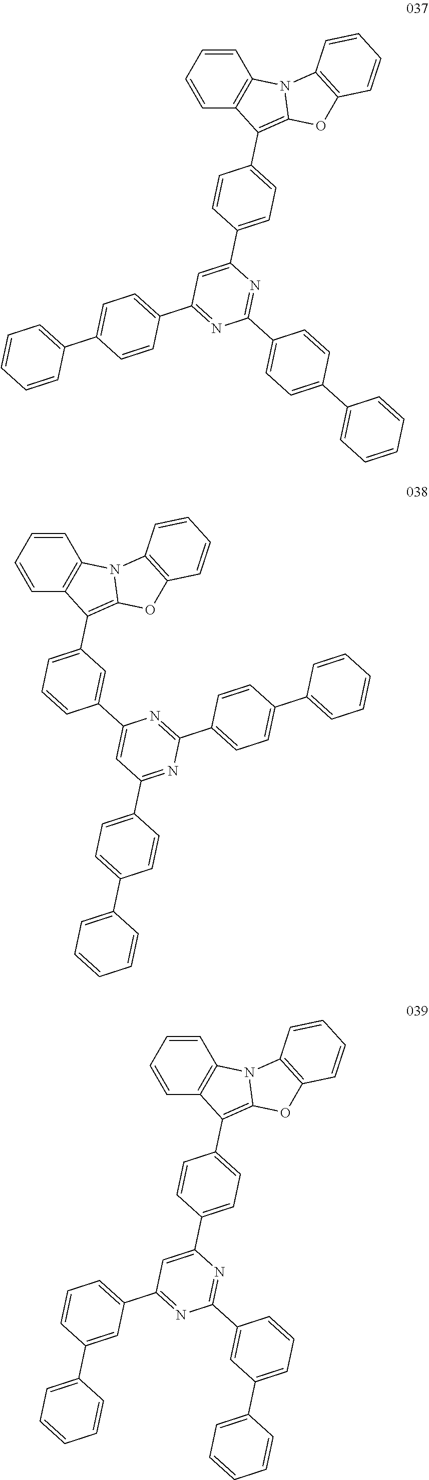 [SMILES: c1ccc(-c2ccc(-c3cc(-c4ccc(-c5c6ccccc6n6c5oc5ccccc56)cc4)nc(-c4ccc(-c5ccccc5)cc4)n3)cc2)cc1 ; c1ccc(-c2ccc(-c3cc(-c4cccc(-c5c6ccccc6n6c5oc5ccccc56)c4)nc(-c4ccc(-c5ccccc5)cc4)n3)cc2)cc1 ; c1ccc(-c2cccc(-c3cc(-c4ccc(-c5c6ccccc6n6c5oc5ccccc56)cc4)nc(-c4cccc(-c5ccccc5)c4)n3)c2)cc1]

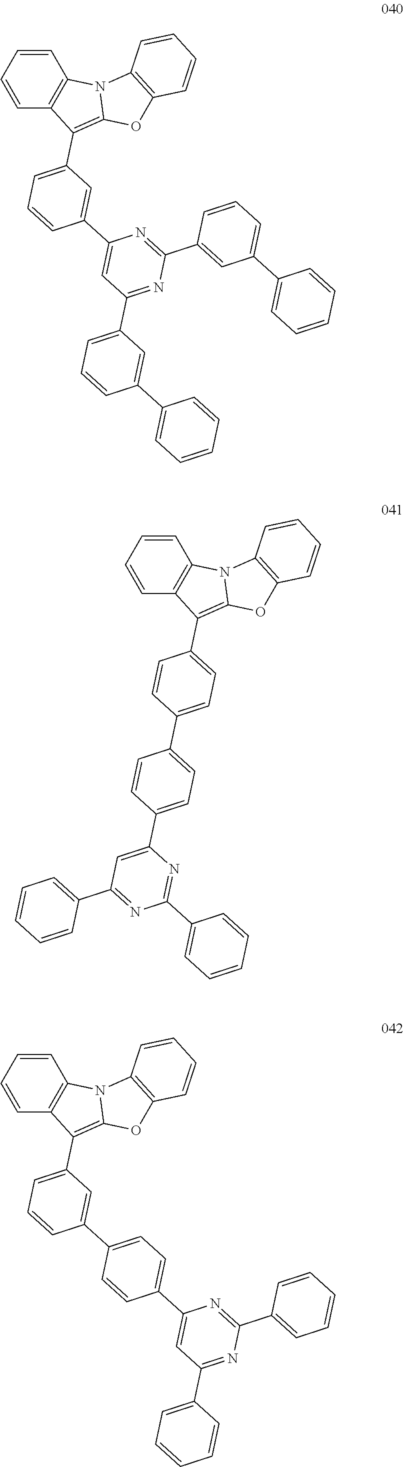 [SMILES: c1ccc(-c2cccc(-c3cc(-c4cccc(-c5c6ccccc6n6c5oc5ccccc56)c4)nc(-c4cccc(-c5ccccc5)c4)n3)c2)cc1 ; c1ccc(-c2cc(-c3ccc(-c4ccc(-c5c6ccccc6n6c5oc5ccccc56)cc4)cc3)nc(-c3ccccc3)n2)cc1 ; c1ccc(-c2cc(-c3ccc(-c4cccc(-c5c6ccccc6n6c5oc5ccccc56)c4)cc3)nc(-c3ccccc3)n2)cc1]

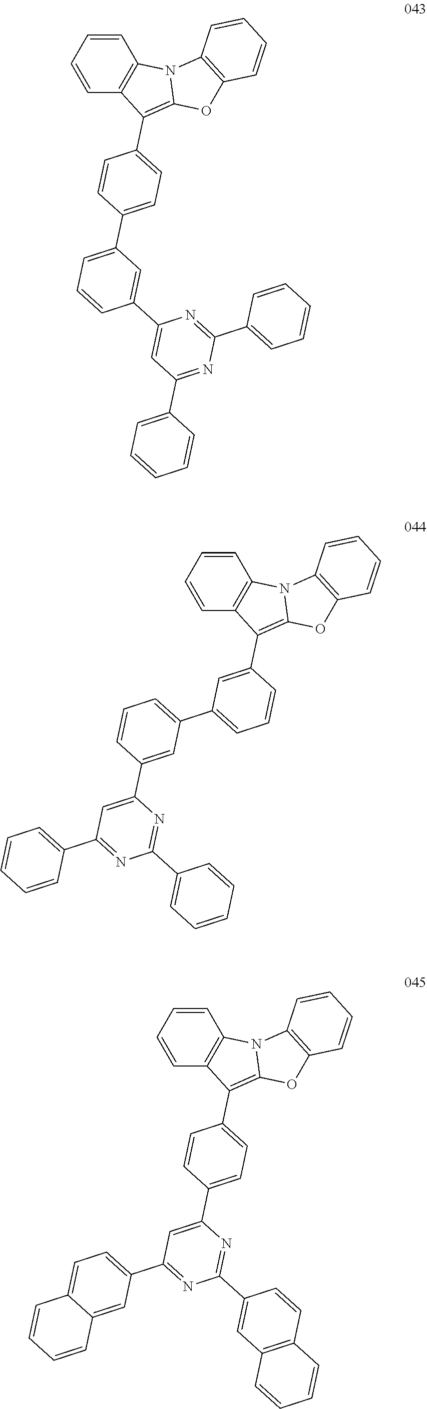 [SMILES: c1ccc(-c2cc(-c3cccc(-c4ccc(-c5c6ccccc6n6c5oc5ccccc56)cc4)c3)nc(-c3ccccc3)n2)cc1 ; c1ccc(-c2cc(-c3cccc(-c4cccc(-c5c6ccccc6n6c5oc5ccccc56)c4)c3)nc(-c3ccccc3)n2)cc1 ; c1ccc2cc(-c3cc(-c4ccc(-c5c6ccccc6n6c5oc5ccccc56)cc4)nc(-c4ccc5ccccc5c4)n3)ccc2c1]

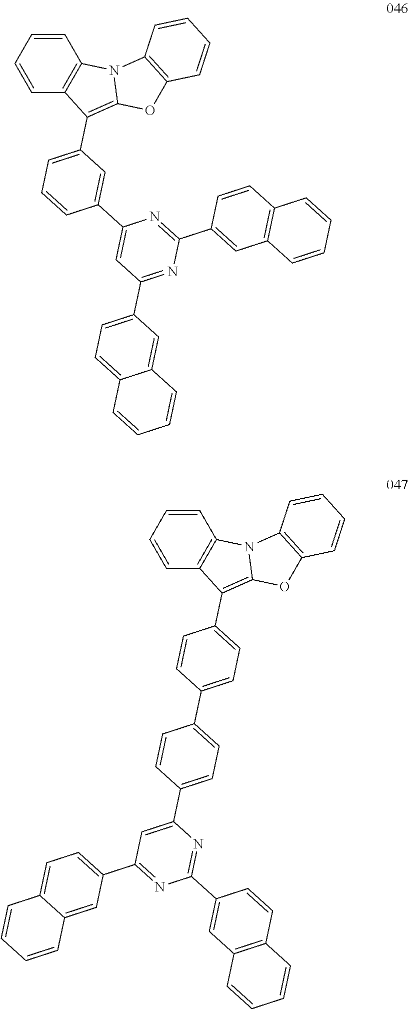 [SMILES: c1cc(-c2cc(-c3ccc4ccccc4c3)nc(-c3ccc4ccccc4c3)n2)cc(-c2c3ccccc3n3c2oc2ccccc23)c1 ; c1ccc2cc(-c3cc(-c4ccc(-c5ccc(-c6c7ccccc7n7c6oc6ccccc67)cc5)cc4)nc(-c4ccc5ccccc5c4)n3)ccc2c1]

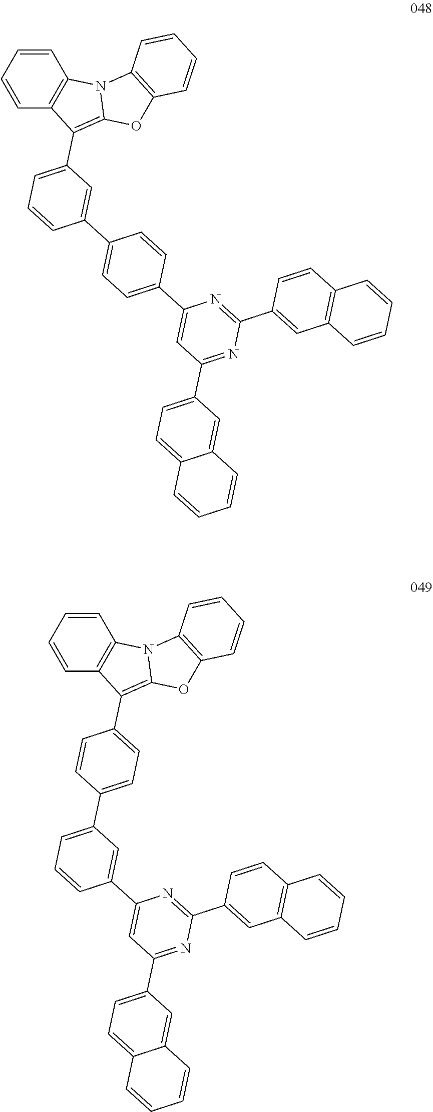 [SMILES: c1cc(-c2ccc(-c3cc(-c4ccc5ccccc5c4)nc(-c4ccc5ccccc5c4)n3)cc2)cc(-c2c3ccccc3n3c2oc2ccccc23)c1 ; c1cc(-c2ccc(-c3c4ccccc4n4c3oc3ccccc34)cc2)cc(-c2cc(-c3ccc4ccccc4c3)nc(-c3ccc4ccccc4c3)n2)c1]

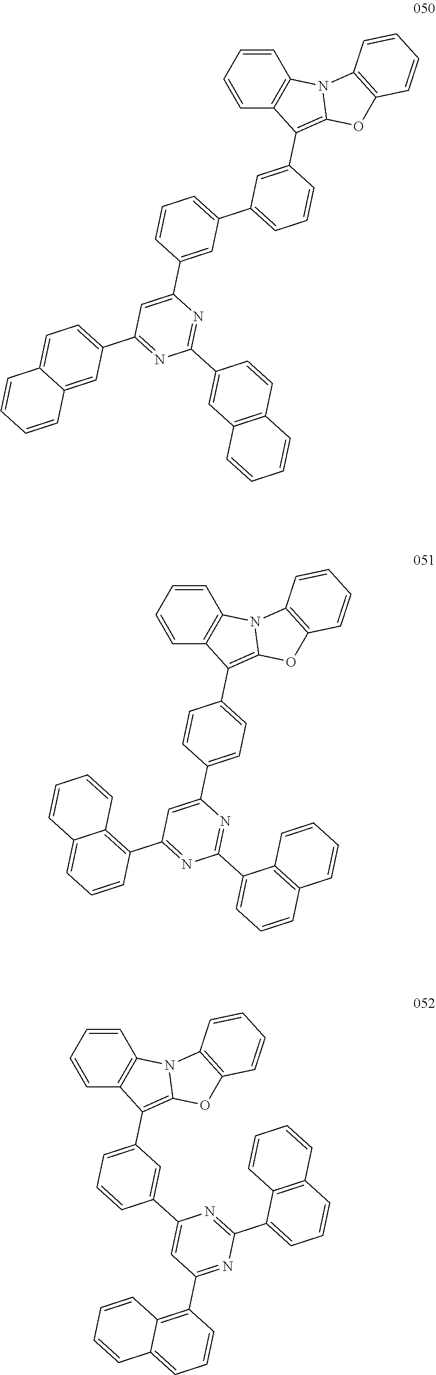 [SMILES: c1cc(-c2cccc(-c3c4ccccc4n4c3oc3ccccc34)c2)cc(-c2cc(-c3ccc4ccccc4c3)nc(-c3ccc4ccccc4c3)n2)c1 ; c1ccc2c(-c3cc(-c4ccc(-c5c6ccccc6n6c5oc5ccccc56)cc4)nc(-c4cccc5ccccc45)n3)cccc2c1 ; c1cc(-c2cc(-c3cccc4ccccc34)nc(-c3cccc4ccccc34)n2)cc(-c2c3ccccc3n3c2oc2ccccc23)c1]

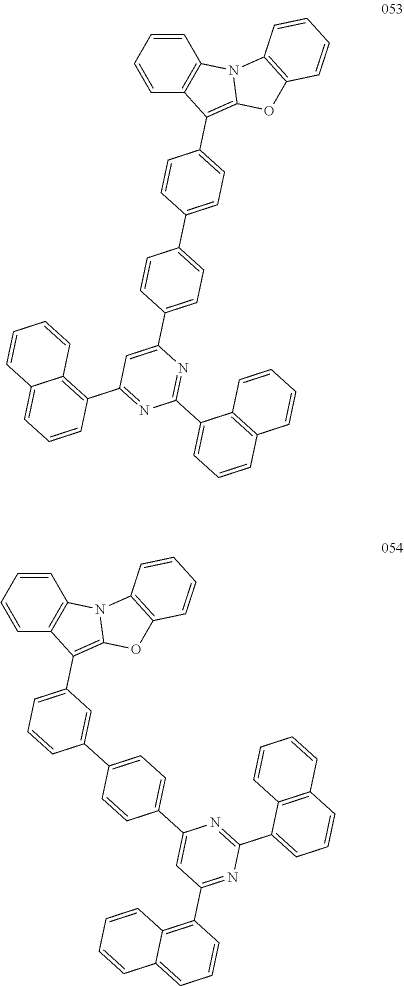 [SMILES: c1ccc2c(-c3cc(-c4ccc(-c5ccc(-c6c7ccccc7n7c6oc6ccccc67)cc5)cc4)nc(-c4cccc5ccccc45)n3)cccc2c1 ; c1cc(-c2ccc(-c3cc(-c4cccc5ccccc45)nc(-c4cccc5ccccc45)n3)cc2)cc(-c2c3ccccc3n3c2oc2ccccc23)c1]

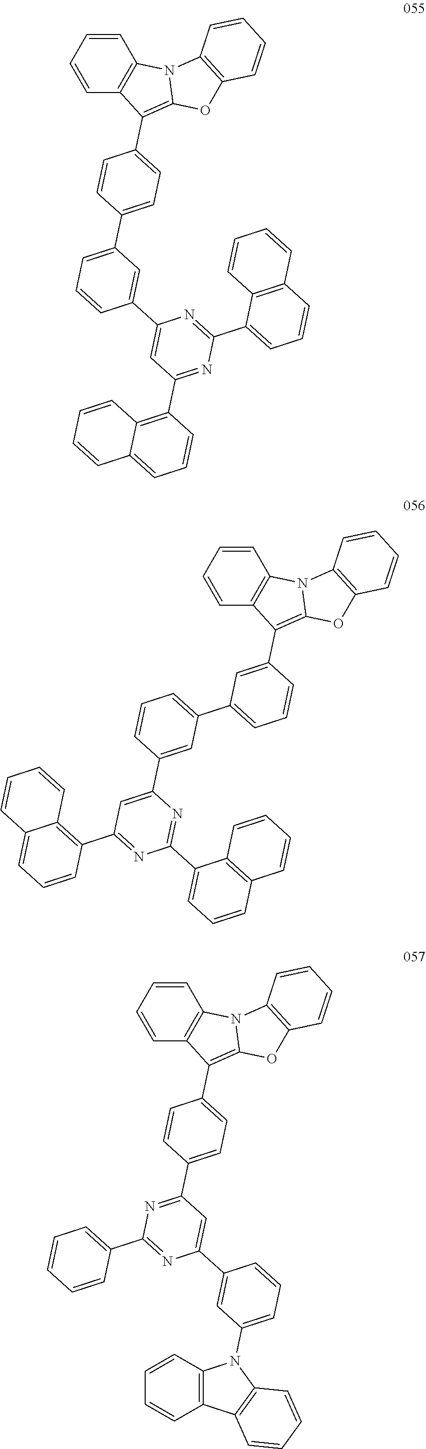 [SMILES: c1cc(-c2ccc(-c3c4ccccc4n4c3oc3ccccc34)cc2)cc(-c2cc(-c3cccc4ccccc34)nc(-c3cccc4ccccc34)n2)c1 ; c1cc(-c2cccc(-c3c4ccccc4n4c3oc3ccccc34)c2)cc(-c2cc(-c3cccc4ccccc34)nc(-c3cccc4ccccc34)n2)c1 ; c1ccc(-c2nc(-c3ccc(-c4c5ccccc5n5c4oc4ccccc45)cc3)cc(-c3cccc(-n4c5ccccc5c5ccccc54)c3)n2)cc1]

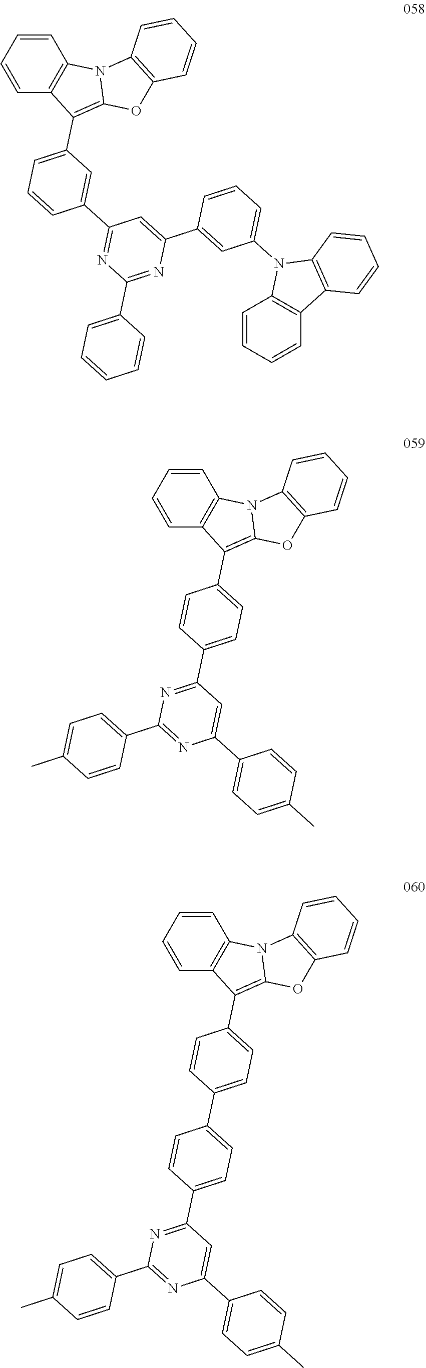 [SMILES: c1ccc(-c2nc(-c3cccc(-c4c5ccccc5n5c4oc4ccccc45)c3)cc(-c3cccc(-n4c5ccccc5c5ccccc54)c3)n2)cc1 ; Cc1ccc(-c2cc(-c3ccc(-c4c5ccccc5n5c4oc4ccccc45)cc3)nc(-c3ccc(C)cc3)n2)cc1 ; Cc1ccc(-c2cc(-c3ccc(-c4ccc(-c5c6ccccc6n6c5oc5ccccc56)cc4)cc3)nc(-c3ccc(C)cc3)n2)cc1]

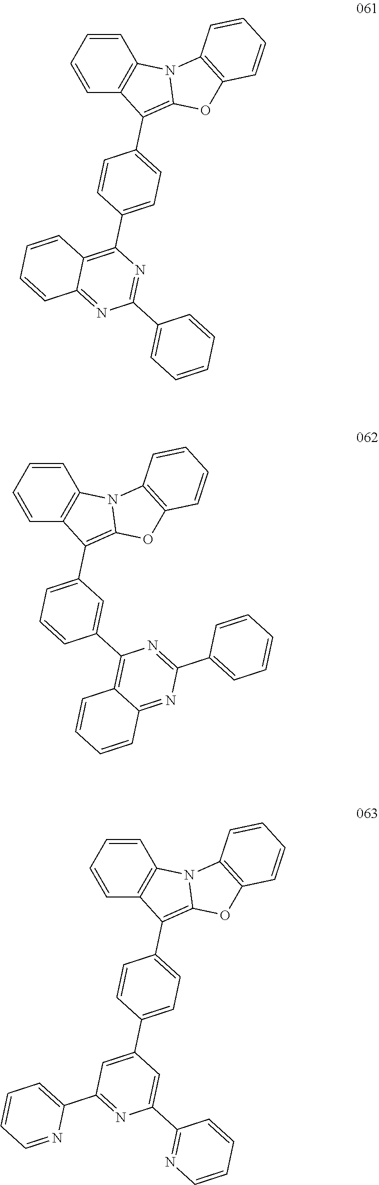 [SMILES: c1ccc(-c2nc(-c3ccc(-c4c5ccccc5n5c4oc4ccccc45)cc3)c3ccccc3n2)cc1 ; c1ccc(-c2nc(-c3cccc(-c4c5ccccc5n5c4oc4ccccc45)c3)c3ccccc3n2)cc1 ; c1ccc(-c2cc(-c3ccc(-c4c5ccccc5n5c4oc4ccccc45)cc3)cc(-c3ccccn3)n2)nc1]

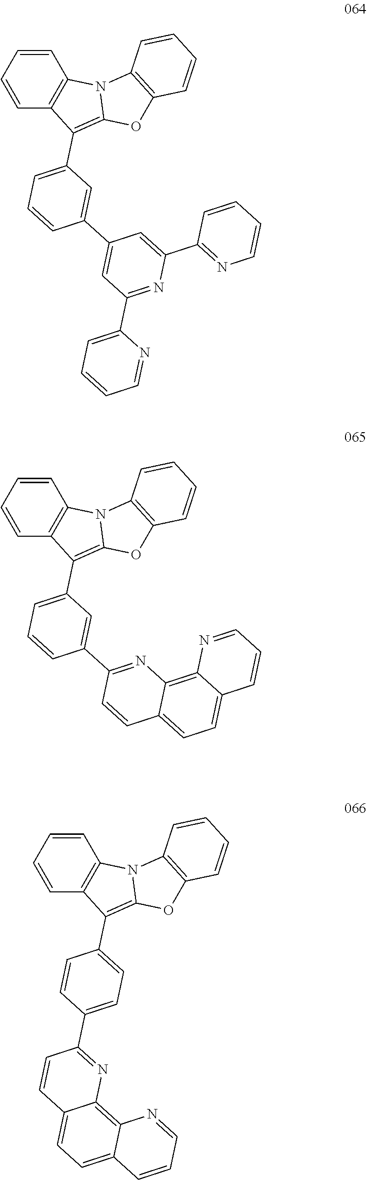 [SMILES: c1ccc(-c2cc(-c3cccc(-c4c5ccccc5n5c4oc4ccccc45)c3)cc(-c3ccccn3)n2)nc1 ; c1cc(-c2ccc3ccc4cccnc4c3n2)cc(-c2c3ccccc3n3c2oc2ccccc23)c1 ; c1cnc2c(c1)ccc1ccc(-c3ccc(-c4c5ccccc5n5c4oc4ccccc45)cc3)nc12]

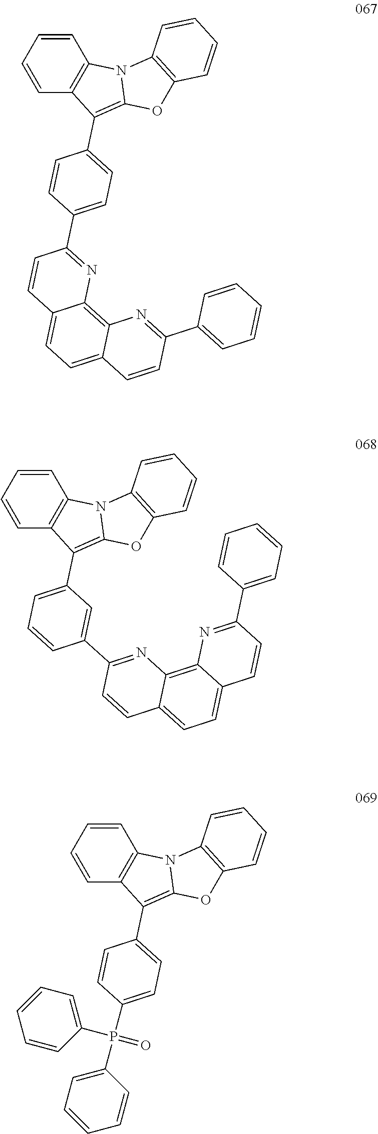 [SMILES: c1ccc(-c2ccc3ccc4ccc(-c5ccc(-c6c7ccccc7n7c6oc6ccccc67)cc5)nc4c3n2)cc1 ; c1ccc(-c2ccc3ccc4ccc(-c5cccc(-c6c7ccccc7n7c6oc6ccccc67)c5)nc4c3n2)cc1 ; O=P(c1ccccc1)(c1ccccc1)c1ccc(-c2c3ccccc3n3c2oc2ccccc23)cc1]

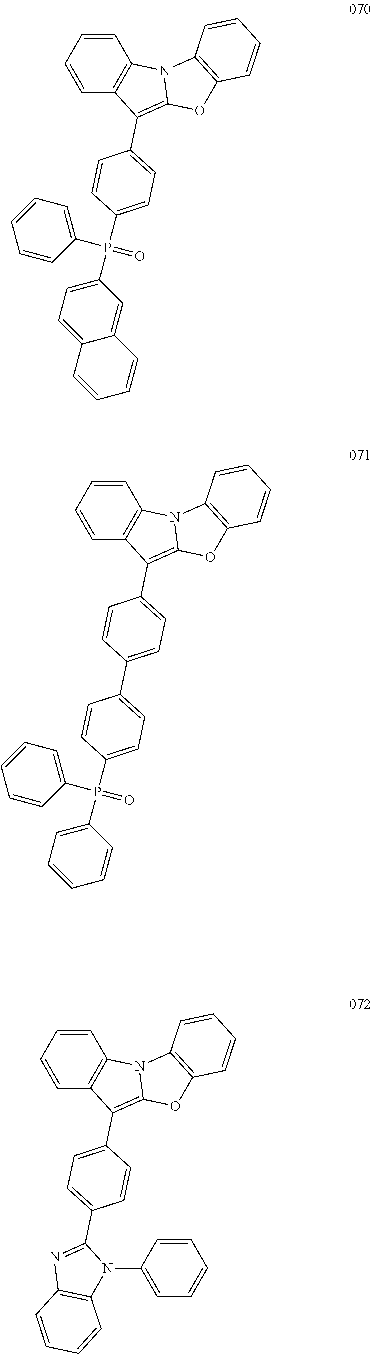 [SMILES: O=P(c1ccccc1)(c1ccc(-c2c3ccccc3n3c2oc2ccccc23)cc1)c1ccc2ccccc2c1 ; O=P(c1ccccc1)(c1ccccc1)c1ccc(-c2ccc(-c3c4ccccc4n4c3oc3ccccc34)cc2)cc1 ; c1ccc(-n2c(-c3ccc(-c4c5ccccc5n5c4oc4ccccc45)cc3)nc3ccccc32)cc1]

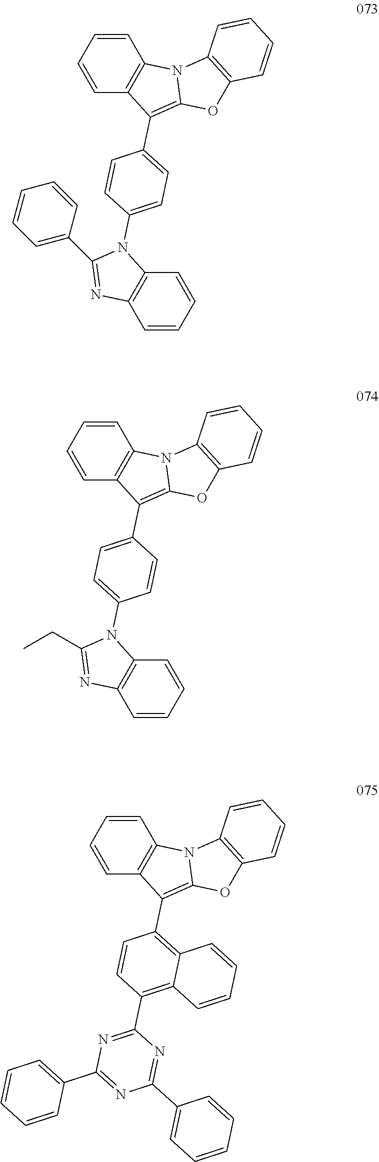 [SMILES: c1ccc(-c2nc3ccccc3n2-c2ccc(-c3c4ccccc4n4c3oc3ccccc34)cc2)cc1 ; CCc1nc2ccccc2n1-c1ccc(-c2c3ccccc3n3c2oc2ccccc23)cc1 ; c1ccc(-c2nc(-c3ccccc3)nc(-c3ccc(-c4c5ccccc5n5c4oc4ccccc45)c4ccccc34)n2)cc1]

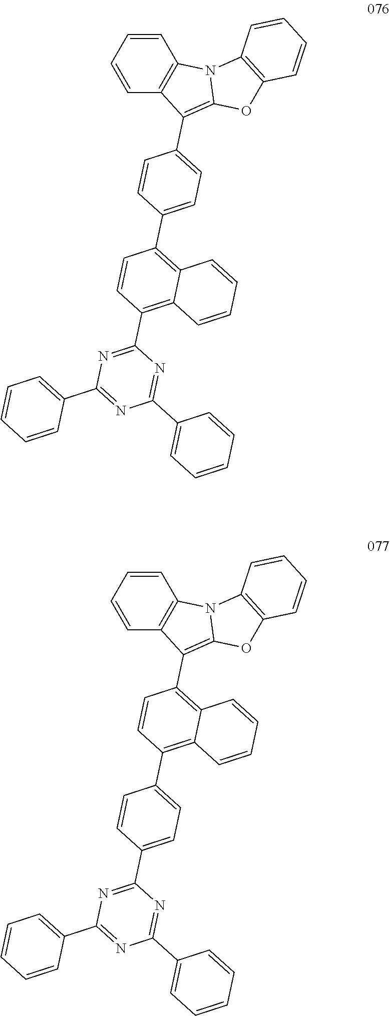 [SMILES: c1ccc(-c2nc(-c3ccccc3)nc(-c3ccc(-c4ccc(-c5c6ccccc6n6c5oc5ccccc56)cc4)c4ccccc34)n2)cc1 ; c1ccc(-c2nc(-c3ccccc3)nc(-c3ccc(-c4ccc(-c5c6ccccc6n6c5oc5ccccc56)c5ccccc45)cc3)n2)cc1]

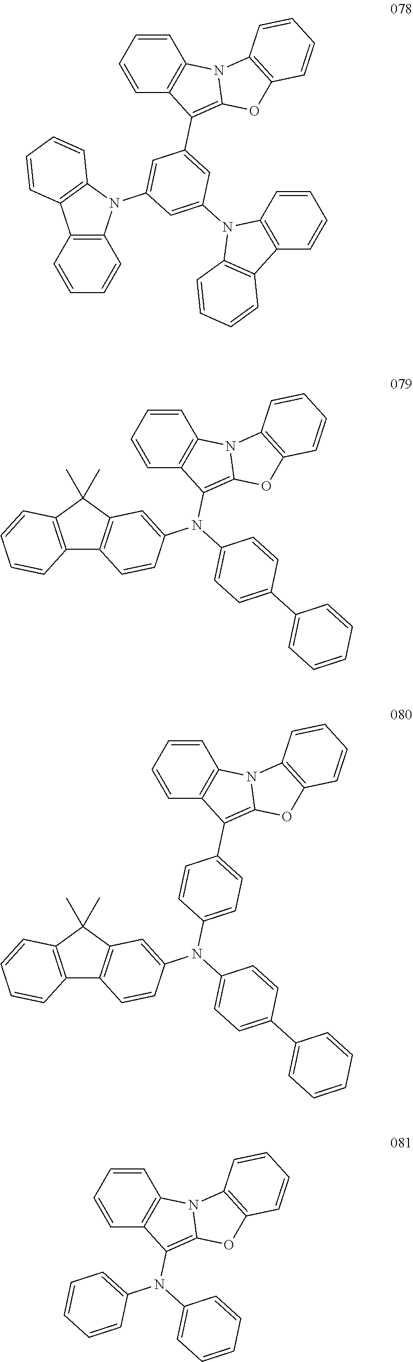 [SMILES: c1ccc2c(c1)oc1c(-c3cc(-n4c5ccccc5c5ccccc54)cc(-n4c5ccccc5c5ccccc54)c3)c3ccccc3n12 ; CC1(C)c2ccccc2-c2ccc(N(c3ccc(-c4ccccc4)cc3)c3c4ccccc4n4c3oc3ccccc34)cc21 ; CC1(C)c2ccccc2-c2ccc(N(c3ccc(-c4ccccc4)cc3)c3ccc(-c4c5ccccc5n5c4oc4ccccc45)cc3)cc21 ; c1ccc(N(c2ccccc2)c2c3ccccc3n3c2oc2ccccc23)cc1]

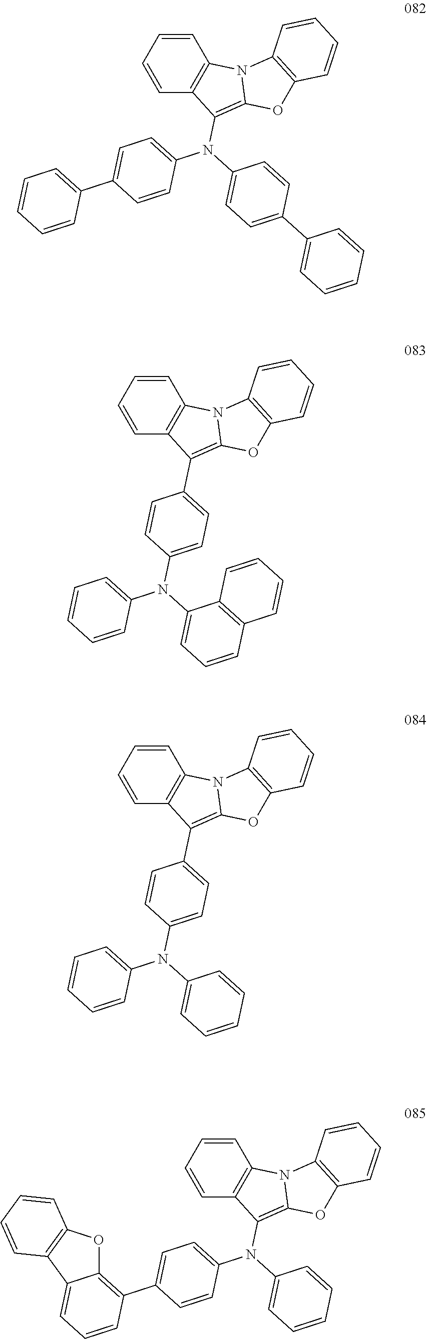 [SMILES: c1ccc(-c2ccc(N(c3ccc(-c4ccccc4)cc3)c3c4ccccc4n4c3oc3ccccc34)cc2)cc1 ; c1ccc(N(c2ccc(-c3c4ccccc4n4c3oc3ccccc34)cc2)c2cccc3ccccc23)cc1 ; c1ccc(N(c2ccccc2)c2ccc(-c3c4ccccc4n4c3oc3ccccc34)cc2)cc1 ; c1ccc(N(c2ccc(-c3cccc4c3oc3ccccc34)cc2)c2c3ccccc3n3c2oc2ccccc23)cc1]

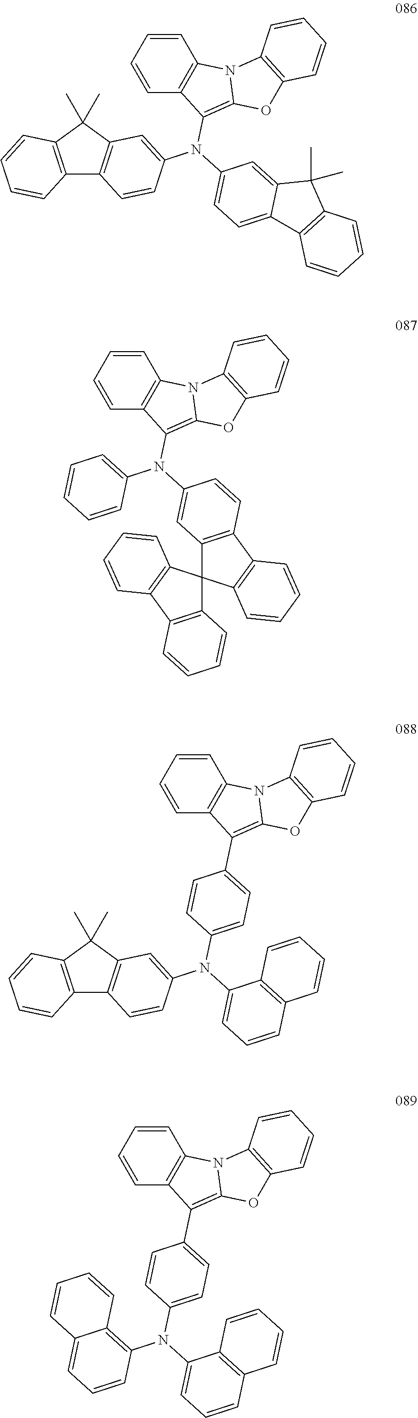 [SMILES: CC1(C)c2ccccc2-c2ccc(N(c3ccc4c(c3)C(C)(C)c3ccccc3-4)c3c4ccccc4n4c3oc3ccccc34)cc21 ; c1ccc(N(c2ccc3c(c2)C2(c4ccccc4-c4ccccc42)c2ccccc2-3)c2c3ccccc3n3c2oc2ccccc23)cc1 ; CC1(C)c2ccccc2-c2ccc(N(c3ccc(-c4c5ccccc5n5c4oc4ccccc45)cc3)c3cccc4ccccc34)cc21 ; c1ccc2c(N(c3ccc(-c4c5ccccc5n5c4oc4ccccc45)cc3)c3cccc4ccccc34)cccc2c1]

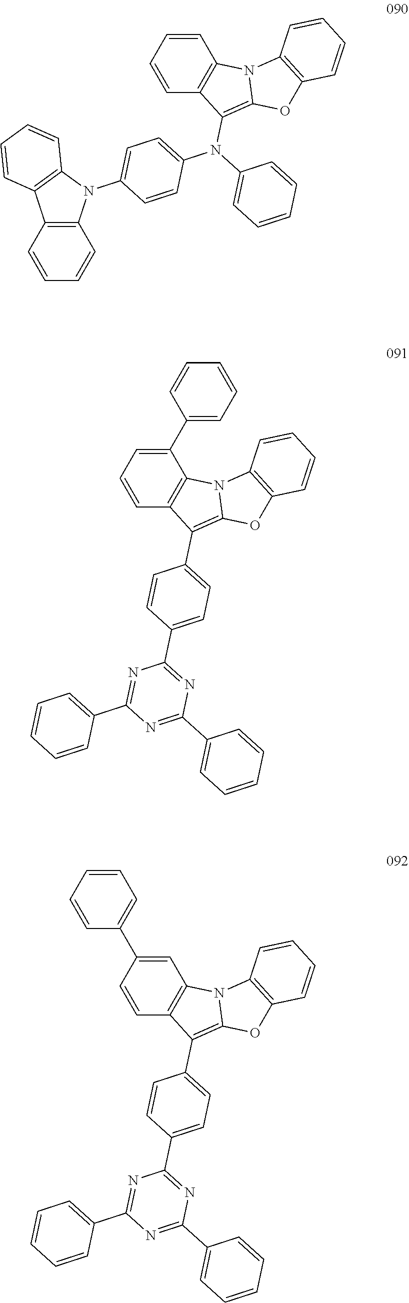 [SMILES: c1ccc(N(c2ccc(-n3c4ccccc4c4ccccc43)cc2)c2c3ccccc3n3c2oc2ccccc23)cc1 ; c1ccc(-c2nc(-c3ccccc3)nc(-c3ccc(-c4c5cccc(-c6ccccc6)c5n5c4oc4ccccc45)cc3)n2)cc1 ; c1ccc(-c2ccc3c(-c4ccc(-c5nc(-c6ccccc6)nc(-c6ccccc6)n5)cc4)c4oc5ccccc5n4c3c2)cc1]

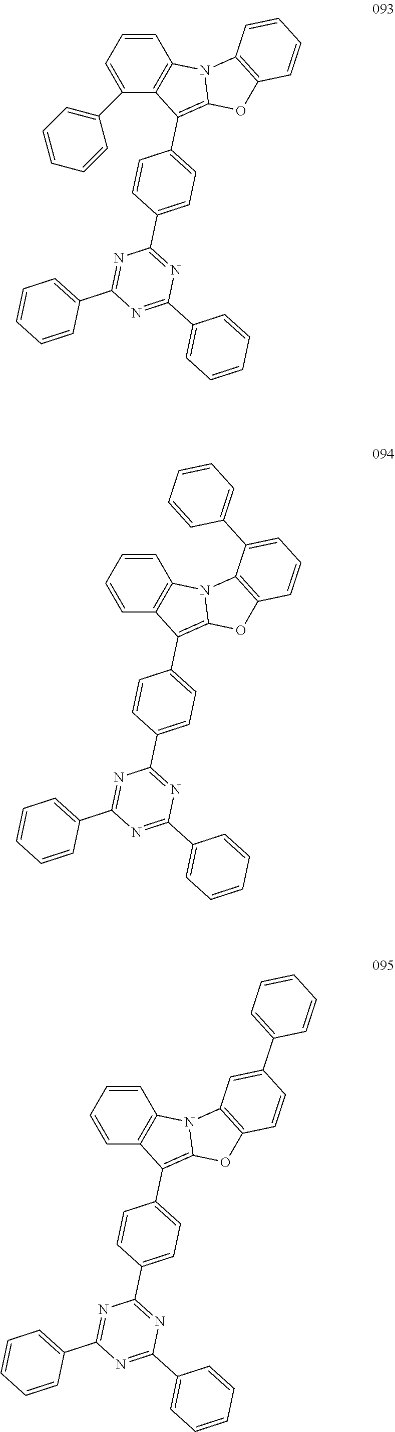 [SMILES: c1ccc(-c2nc(-c3ccccc3)nc(-c3ccc(-c4c5c(-c6ccccc6)cccc5n5c4oc4ccccc45)cc3)n2)cc1 ; c1ccc(-c2nc(-c3ccccc3)nc(-c3ccc(-c4c5ccccc5n5c4oc4cccc(-c6ccccc6)c45)cc3)n2)cc1 ; c1ccc(-c2ccc3oc4c(-c5ccc(-c6nc(-c7ccccc7)nc(-c7ccccc7)n6)cc5)c5ccccc5n4c3c2)cc1]

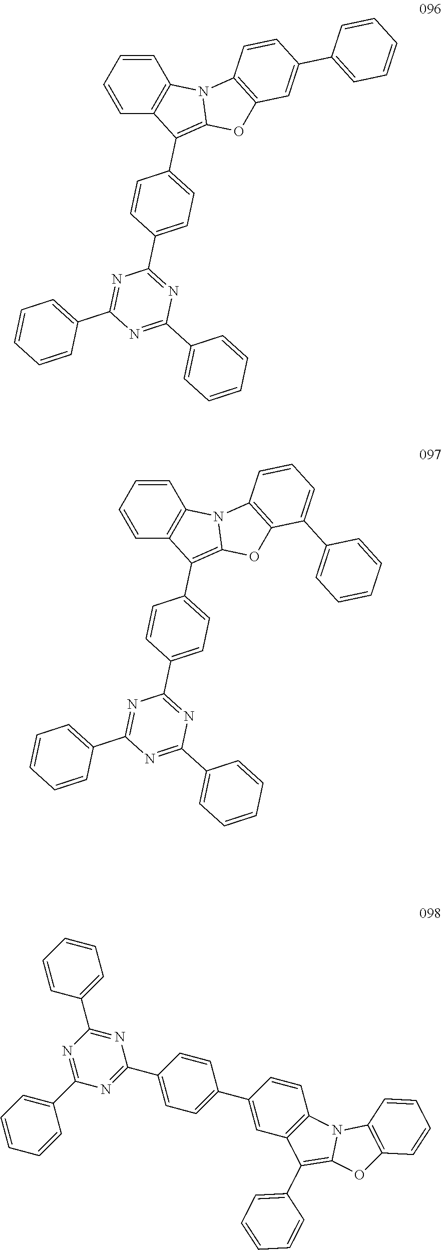 [SMILES: c1ccc(-c2ccc3c(c2)oc2c(-c4ccc(-c5nc(-c6ccccc6)nc(-c6ccccc6)n5)cc4)c4ccccc4n23)cc1 ; c1ccc(-c2nc(-c3ccccc3)nc(-c3ccc(-c4c5ccccc5n5c4oc4c(-c6ccccc6)cccc45)cc3)n2)cc1 ; c1ccc(-c2nc(-c3ccccc3)nc(-c3ccc(-c4ccc5c(c4)c(-c4ccccc4)c4oc6ccccc6n45)cc3)n2)cc1]

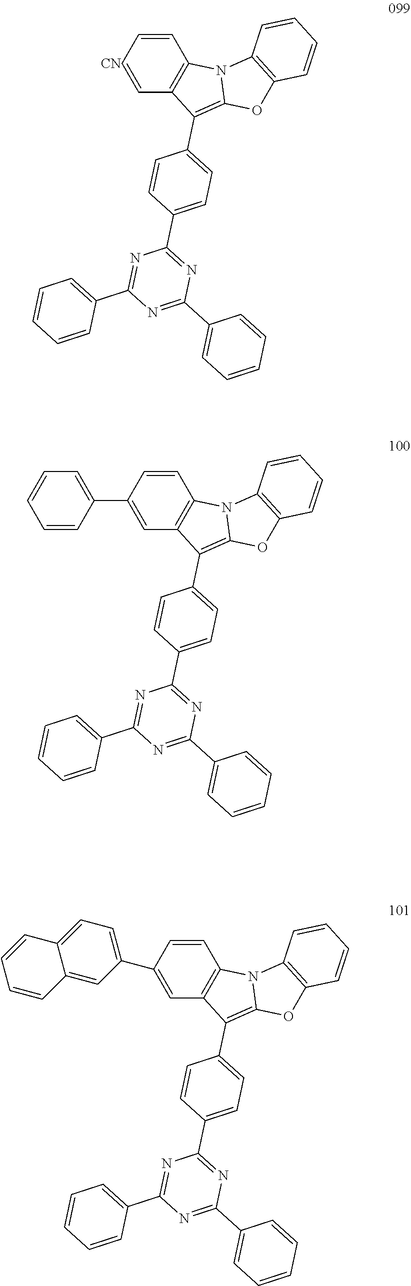

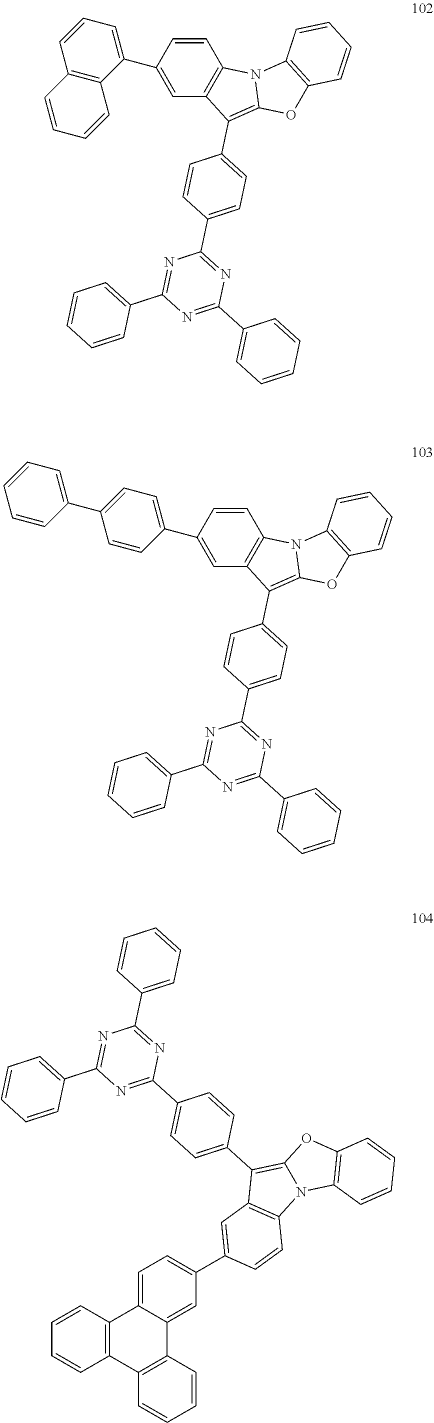 [SMILES: c1ccc(-c2nc(-c3ccccc3)nc(-c3ccc(-c4c5cc(-c6cccc7ccccc67)ccc5n5c4oc4ccccc45)cc3)n2)cc1 ; c1ccc(-c2ccc(-c3ccc4c(c3)c(-c3ccc(-c5nc(-c6ccccc6)nc(-c6ccccc6)n5)cc3)c3oc5ccccc5n34)cc2)cc1 ; c1ccc(-c2nc(-c3ccccc3)nc(-c3ccc(-c4c5cc(-c6ccc7c8ccccc8c8ccccc8c7c6)ccc5n5c4oc4ccccc45)cc3)n2)cc1]

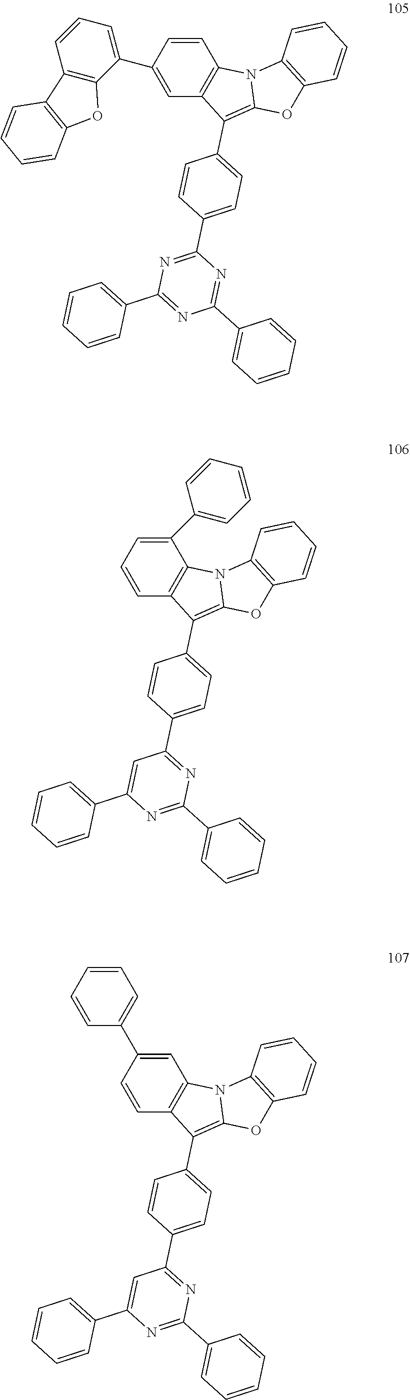 [SMILES: c1ccc(-c2nc(-c3ccccc3)nc(-c3ccc(-c4c5cc(-c6cccc7c6oc6ccccc67)ccc5n5c4oc4ccccc45)cc3)n2)cc1 ; c1ccc(-c2cc(-c3ccc(-c4c5cccc(-c6ccccc6)c5n5c4oc4ccccc45)cc3)nc(-c3ccccc3)n2)cc1 ; c1ccc(-c2ccc3c(-c4ccc(-c5cc(-c6ccccc6)nc(-c6ccccc6)n5)cc4)c4oc5ccccc5n4c3c2)cc1]

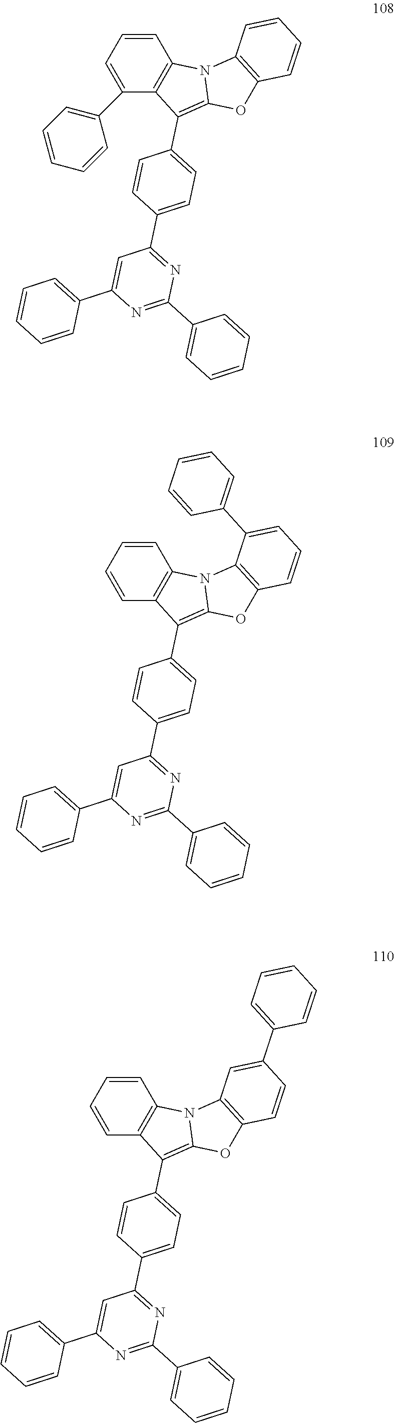 [SMILES: c1ccc(-c2cc(-c3ccc(-c4c5c(-c6ccccc6)cccc5n5c4oc4ccccc45)cc3)nc(-c3ccccc3)n2)cc1 ; c1ccc(-c2cc(-c3ccc(-c4c5ccccc5n5c4oc4cccc(-c6ccccc6)c45)cc3)nc(-c3ccccc3)n2)cc1 ; c1ccc(-c2ccc3oc4c(-c5ccc(-c6cc(-c7ccccc7)nc(-c7ccccc7)n6)cc5)c5ccccc5n4c3c2)cc1]

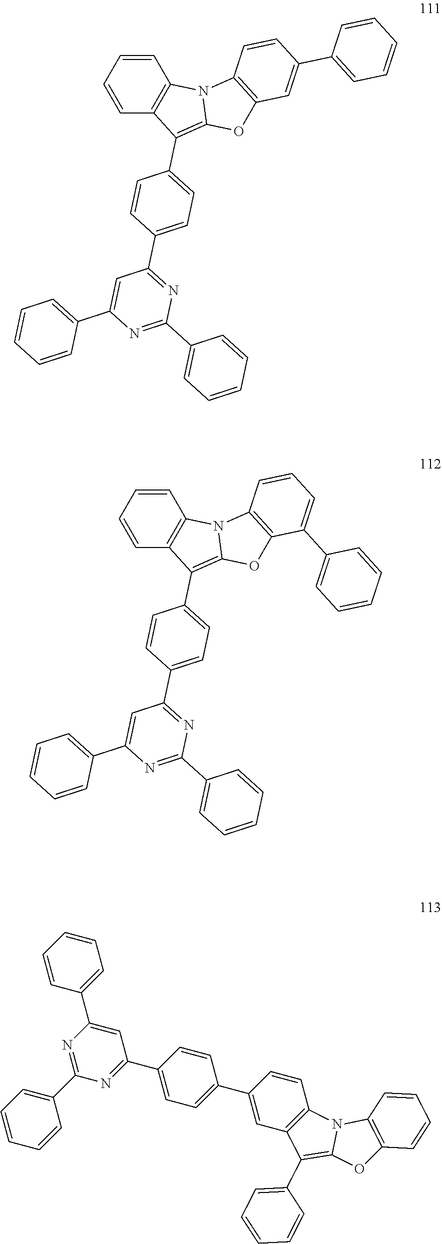 [SMILES: c1ccc(-c2ccc3c(c2)oc2c(-c4ccc(-c5cc(-c6ccccc6)nc(-c6ccccc6)n5)cc4)c4ccccc4n23)cc1 ; c1ccc(-c2cc(-c3ccc(-c4c5ccccc5n5c4oc4c(-c6ccccc6)cccc45)cc3)nc(-c3ccccc3)n2)cc1 ; c1ccc(-c2cc(-c3ccc(-c4ccc5c(c4)c(-c4ccccc4)c4oc6ccccc6n45)cc3)nc(-c3ccccc3)n2)cc1]

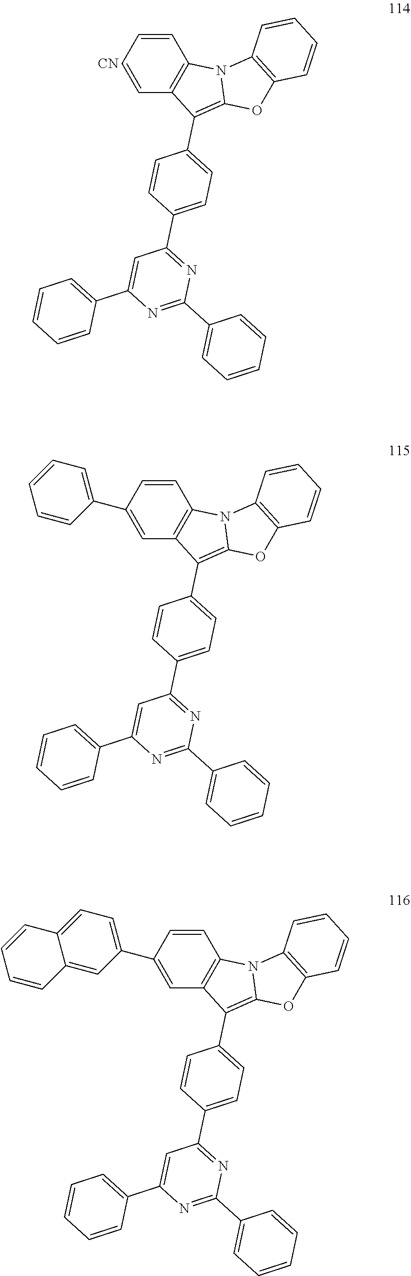

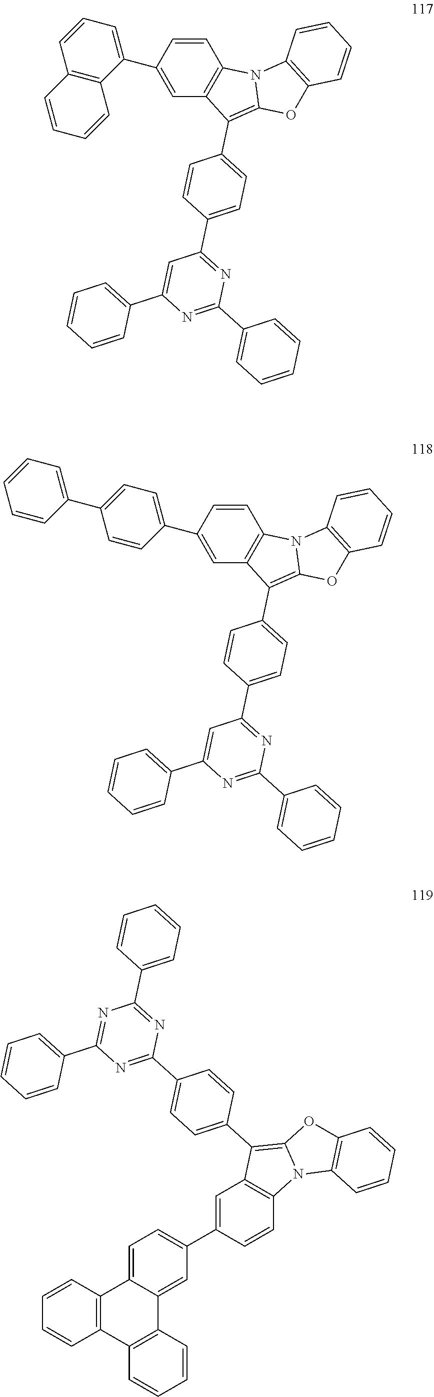 [SMILES: c1ccc(-c2cc(-c3ccc(-c4c5cc(-c6cccc7ccccc67)ccc5n5c4oc4ccccc45)cc3)nc(-c3ccccc3)n2)cc1 ; c1ccc(-c2ccc(-c3ccc4c(c3)c(-c3ccc(-c5cc(-c6ccccc6)nc(-c6ccccc6)n5)cc3)c3oc5ccccc5n34)cc2)cc1 ; c1ccc(-c2nc(-c3ccccc3)nc(-c3ccc(-c4c5cc(-c6ccc7c8ccccc8c8ccccc8c7c6)ccc5n5c4oc4ccccc45)cc3)n2)cc1]

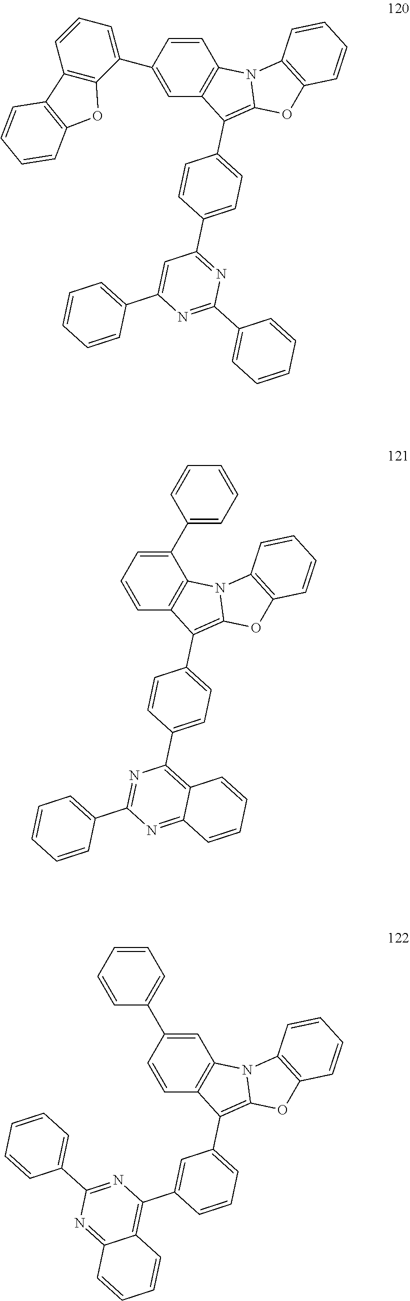 [SMILES: c1ccc(-c2cc(-c3ccc(-c4c5cc(-c6cccc7c6oc6ccccc67)ccc5n5c4oc4ccccc45)cc3)nc(-c3ccccc3)n2)cc1 ; c1ccc(-c2nc(-c3ccc(-c4c5cccc(-c6ccccc6)c5n5c4oc4ccccc45)cc3)c3ccccc3n2)cc1 ; c1ccc(-c2ccc3c(-c4cccc(-c5nc(-c6ccccc6)nc6ccccc56)c4)c4oc5ccccc5n4c3c2)cc1]

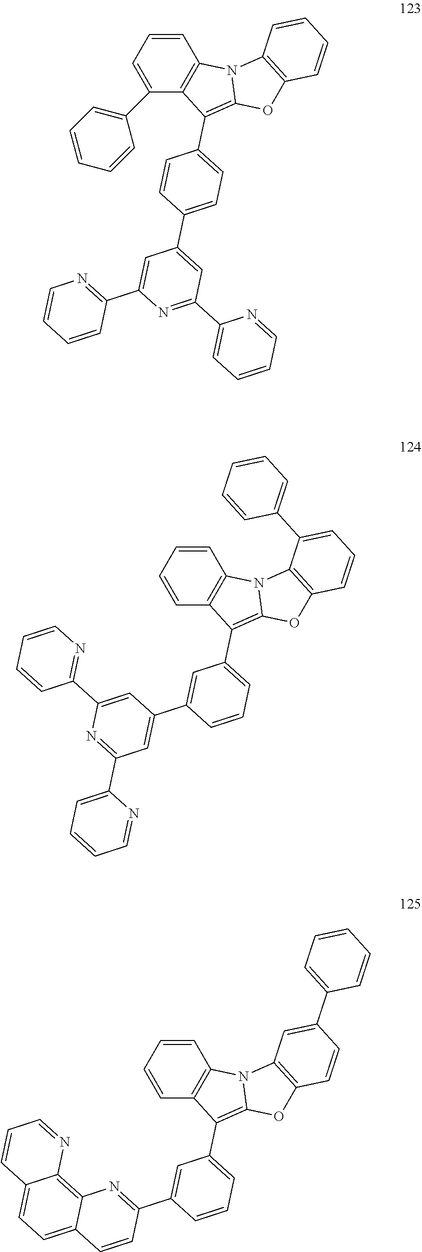 [SMILES: c1ccc(-c2cccc3c2c(-c2ccc(-c4cc(-c5ccccn5)nc(-c5ccccn5)c4)cc2)c2oc4ccccc4n23)cc1 ; c1ccc(-c2cccc3oc4c(-c5cccc(-c6cc(-c7ccccn7)nc(-c7ccccn7)c6)c5)c5ccccc5n4c23)cc1 ; c1ccc(-c2ccc3oc4c(-c5cccc(-c6ccc7ccc8cccnc8c7n6)c5)c5ccccc5n4c3c2)cc1]

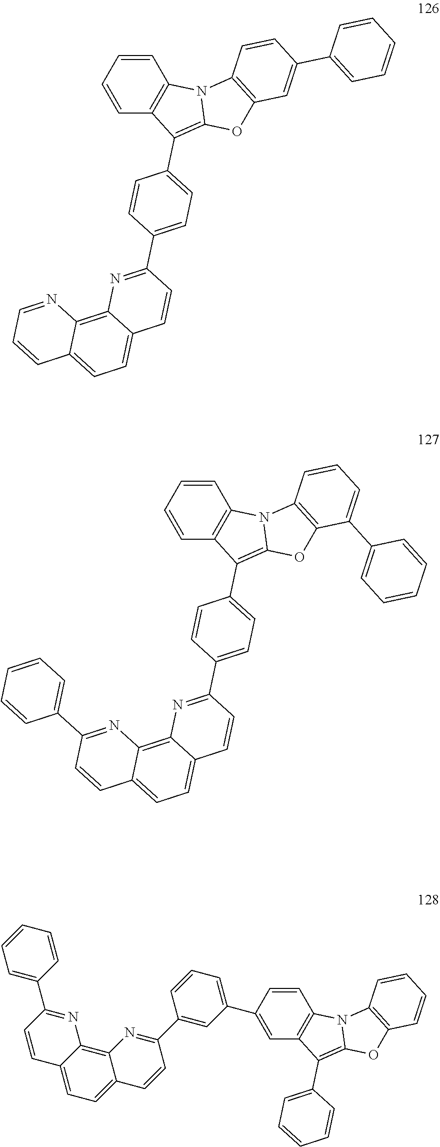 [SMILES: c1ccc(-c2ccc3c(c2)oc2c(-c4ccc(-c5ccc6ccc7cccnc7c6n5)cc4)c4ccccc4n23)cc1 ; c1ccc(-c2ccc3ccc4ccc(-c5ccc(-c6c7ccccc7n7c6oc6c(-c8ccccc8)cccc67)cc5)nc4c3n2)cc1 ; c1ccc(-c2ccc3ccc4ccc(-c5cccc(-c6ccc7c(c6)c(-c6ccccc6)c6oc8ccccc8n67)c5)nc4c3n2)cc1]

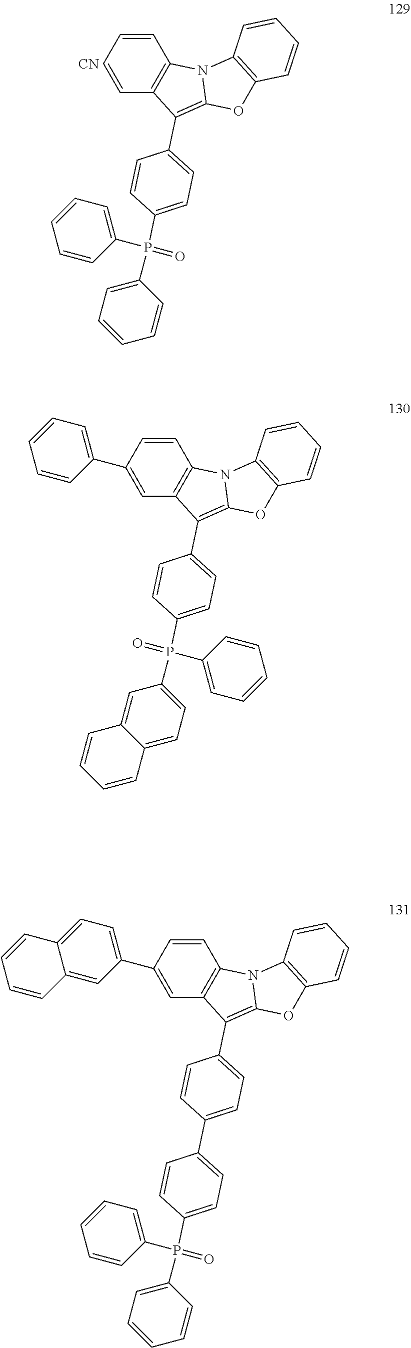

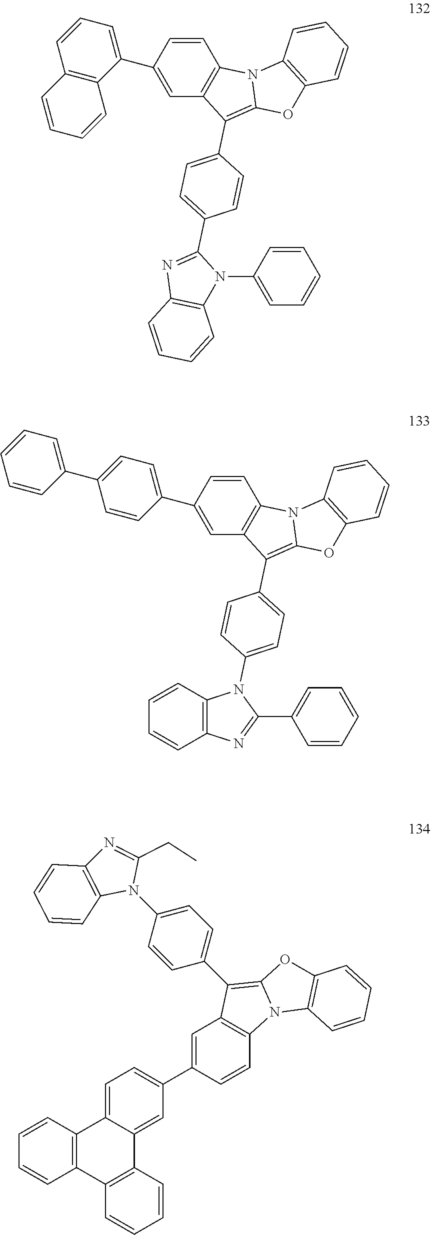 [SMILES: c1ccc(-n2c(-c3ccc(-c4c5cc(-c6cccc7ccccc67)ccc5n5c4oc4ccccc45)cc3)nc3ccccc32)cc1 ; c1ccc(-c2ccc(-c3ccc4c(c3)c(-c3ccc(-n5c(-c6ccccc6)nc6ccccc65)cc3)c3oc5ccccc5n34)cc2)cc1 ; CCc1nc2ccccc2n1-c1ccc(-c2c3cc(-c4ccc5c6ccccc6c6ccccc6c5c4)ccc3n3c2oc2ccccc23)cc1]

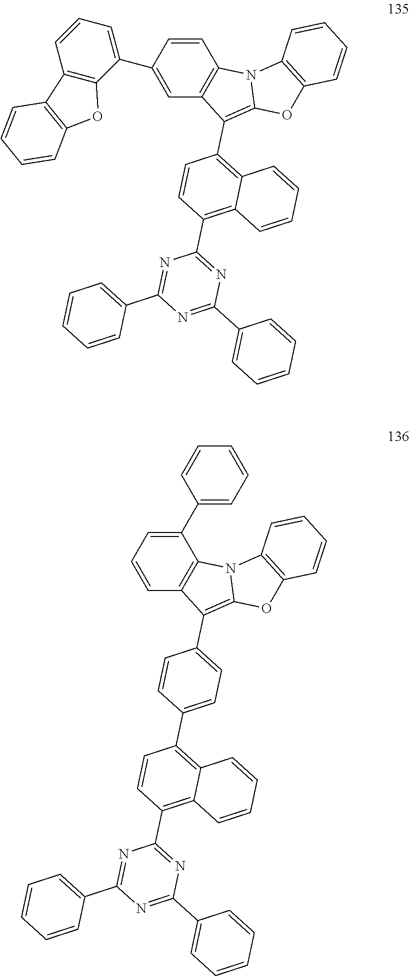 [SMILES: c1ccc(-c2nc(-c3ccccc3)nc(-c3ccc(-c4c5cc(-c6cccc7c6oc6ccccc67)ccc5n5c4oc4ccccc45)c4ccccc34)n2)cc1 ; c1ccc(-c2nc(-c3ccccc3)nc(-c3ccc(-c4ccc(-c5c6cccc(-c7ccccc7)c6n6c5oc5ccccc56)cc4)c4ccccc34)n2)cc1]

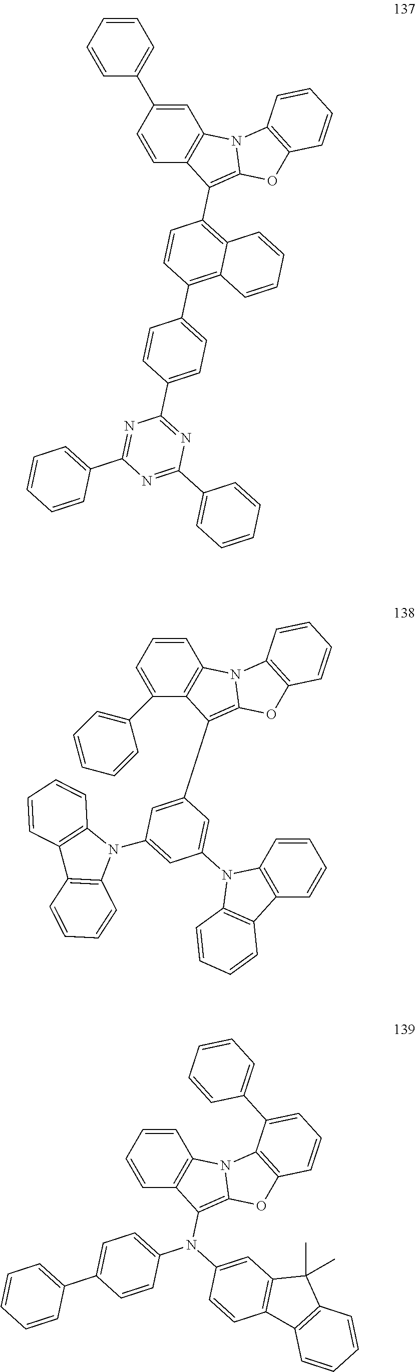 [SMILES: c1ccc(-c2ccc3c(-c4ccc(-c5ccc(-c6nc(-c7ccccc7)nc(-c7ccccc7)n6)cc5)c5ccccc45)c4oc5ccccc5n4c3c2)cc1 ; c1ccc(-c2cccc3c2c(-c2cc(-n4c5ccccc5c5ccccc54)cc(-n4c5ccccc5c5ccccc54)c2)c2oc4ccccc4n23)cc1 ; CC1(C)c2ccccc2-c2ccc(N(c3ccc(-c4ccccc4)cc3)c3c4ccccc4n4c3oc3cccc(-c5ccccc5)c34)cc21]

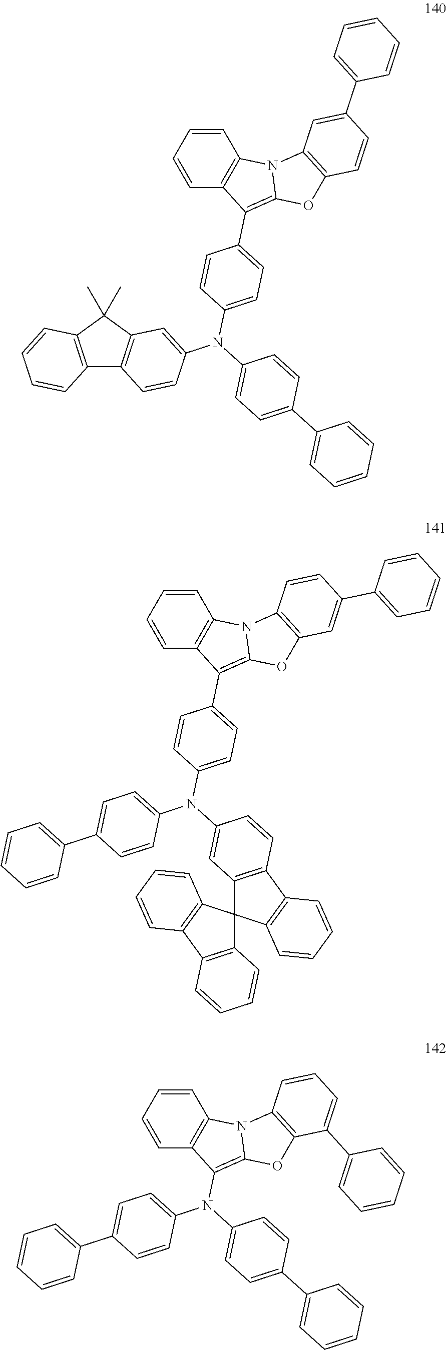 [SMILES: CC1(C)c2ccccc2-c2ccc(N(c3ccc(-c4ccccc4)cc3)c3ccc(-c4c5ccccc5n5c4oc4ccc(-c6ccccc6)cc45)cc3)cc21 ; c1ccc(-c2ccc(N(c3ccc(-c4c5ccccc5n5c4oc4cc(-c6ccccc6)ccc45)cc3)c3ccc4c(c3)C3(c5ccccc5-c5ccccc53)c3ccccc3-4)cc2)cc1 ; c1ccc(-c2ccc(N(c3ccc(-c4ccccc4)cc3)c3c4ccccc4n4c3oc3c(-c5ccccc5)cccc34)cc2)cc1]

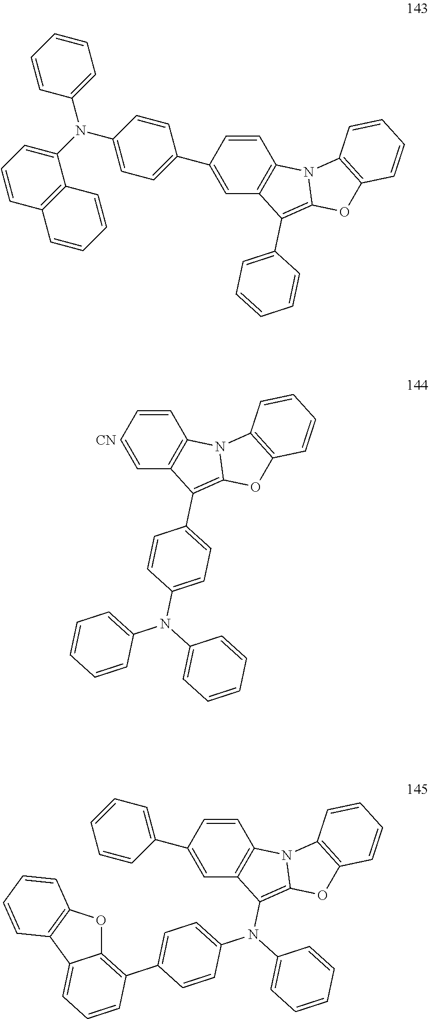

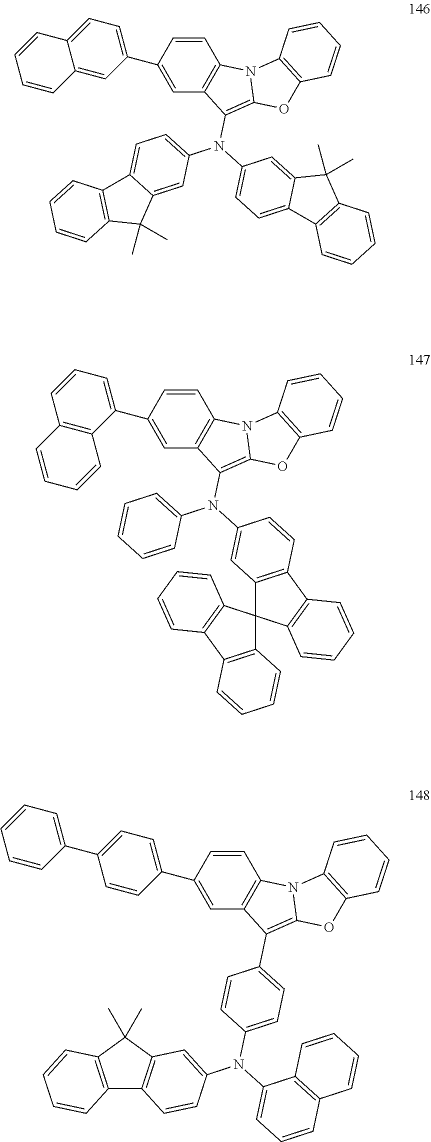 [SMILES: CC1(C)c2ccccc2-c2ccc(N(c3ccc4c(c3)C(C)(C)c3ccccc3-4)c3c4cc(-c5ccc6ccccc6c5)ccc4n4c3oc3ccccc34)cc21 ; c1ccc(N(c2ccc3c(c2)C2(c4ccccc4-c4ccccc42)c2ccccc2-3)c2c3cc(-c4cccc5ccccc45)ccc3n3c2oc2ccccc23)cc1 ; CC1(C)c2ccccc2-c2ccc(N(c3ccc(-c4c5cc(-c6ccc(-c7ccccc7)cc6)ccc5n5c4oc4ccccc45)cc3)c3cccc4ccccc34)cc21]

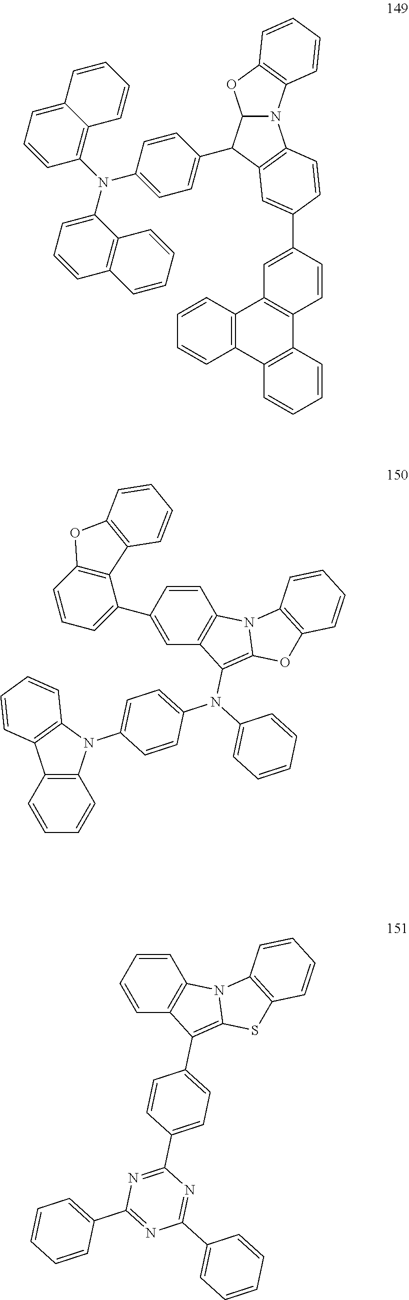 [SMILES: c1ccc2c(c1)OC1C(c3ccc(N(c4cccc5ccccc45)c4cccc5ccccc45)cc3)c3cc(-c4ccc5c6ccccc6c6ccccc6c5c4)ccc3N21 ; c1ccc(N(c2ccc(-n3c4ccccc4c4ccccc43)cc2)c2c3cc(-c4cccc5oc6ccccc6c45)ccc3n3c2oc2ccccc23)cc1 ; c1ccc(-c2nc(-c3ccccc3)nc(-c3ccc(-c4c5ccccc5n5c4sc4ccccc45)cc3)n2)cc1]

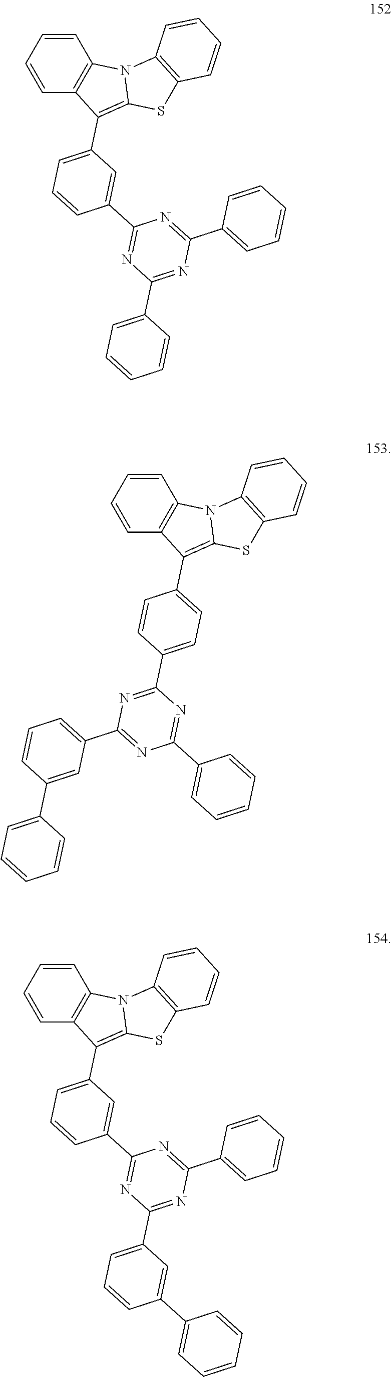 [SMILES: c1ccc(-c2nc(-c3ccccc3)nc(-c3cccc(-c4c5ccccc5n5c4sc4ccccc45)c3)n2)cc1 ; c1ccc(-c2cccc(-c3nc(-c4ccccc4)nc(-c4ccc(-c5c6ccccc6n6c5sc5ccccc56)cc4)n3)c2)cc1 ; c1ccc(-c2cccc(-c3nc(-c4ccccc4)nc(-c4cccc(-c5c6ccccc6n6c5sc5ccccc56)c4)n3)c2)cc1]

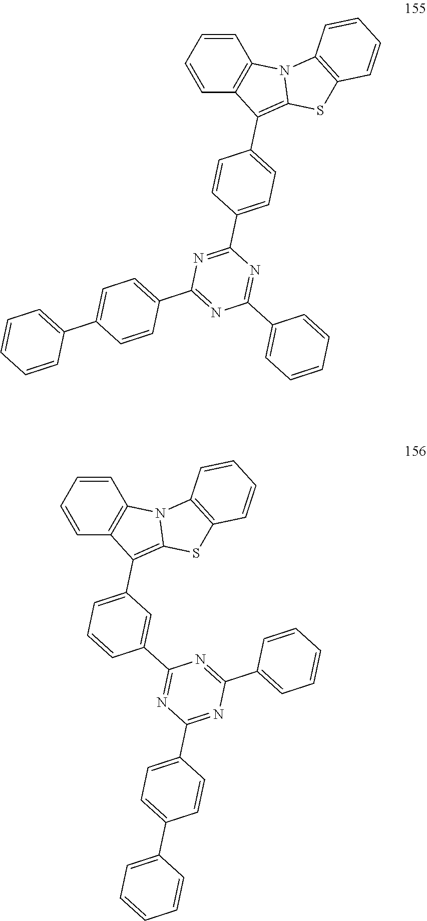 [SMILES: c1ccc(-c2ccc(-c3nc(-c4ccccc4)nc(-c4ccc(-c5c6ccccc6n6c5sc5ccccc56)cc4)n3)cc2)cc1 ; c1ccc(-c2ccc(-c3nc(-c4ccccc4)nc(-c4cccc(-c5c6ccccc6n6c5sc5ccccc56)c4)n3)cc2)cc1]

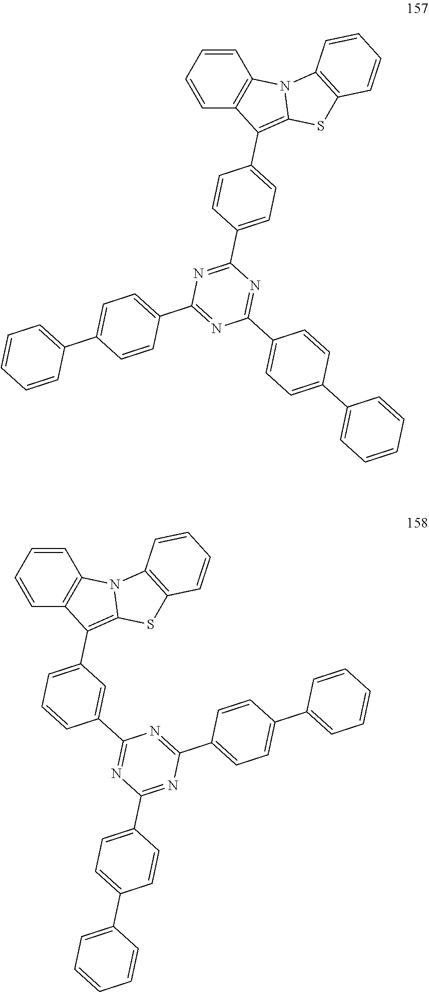 [SMILES: c1ccc(-c2ccc(-c3nc(-c4ccc(-c5ccccc5)cc4)nc(-c4ccc(-c5c6ccccc6n6c5sc5ccccc56)cc4)n3)cc2)cc1 ; c1ccc(-c2ccc(-c3nc(-c4ccc(-c5ccccc5)cc4)nc(-c4cccc(-c5c6ccccc6n6c5sc5ccccc56)c4)n3)cc2)cc1]

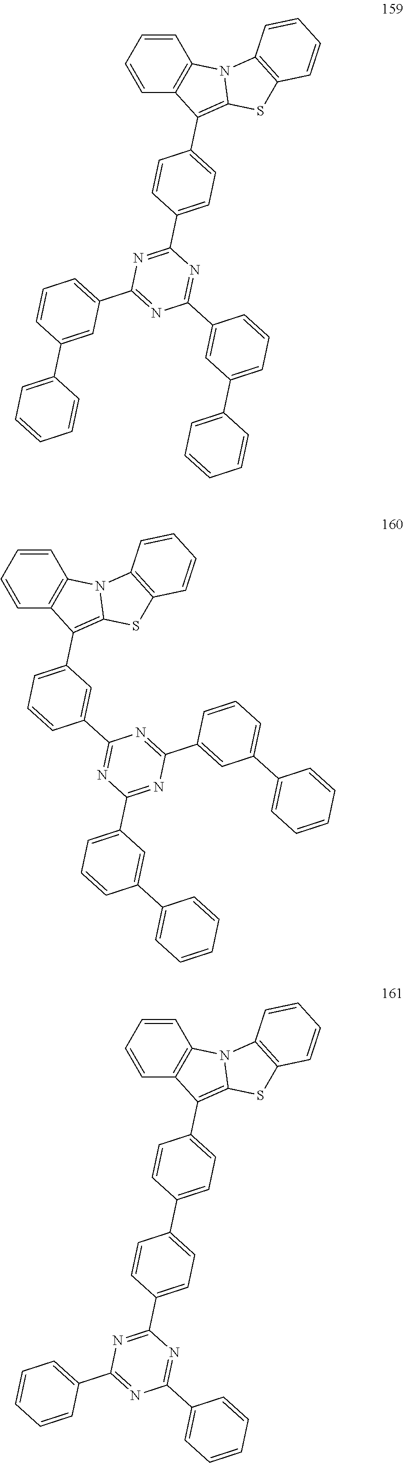 [SMILES: c1ccc(-c2cccc(-c3nc(-c4ccc(-c5c6ccccc6n6c5sc5ccccc56)cc4)nc(-c4cccc(-c5ccccc5)c4)n3)c2)cc1 ; c1ccc(-c2cccc(-c3nc(-c4cccc(-c5ccccc5)c4)nc(-c4cccc(-c5c6ccccc6n6c5sc5ccccc56)c4)n3)c2)cc1 ; c1ccc(-c2nc(-c3ccccc3)nc(-c3ccc(-c4ccc(-c5c6ccccc6n6c5sc5ccccc56)cc4)cc3)n2)cc1]

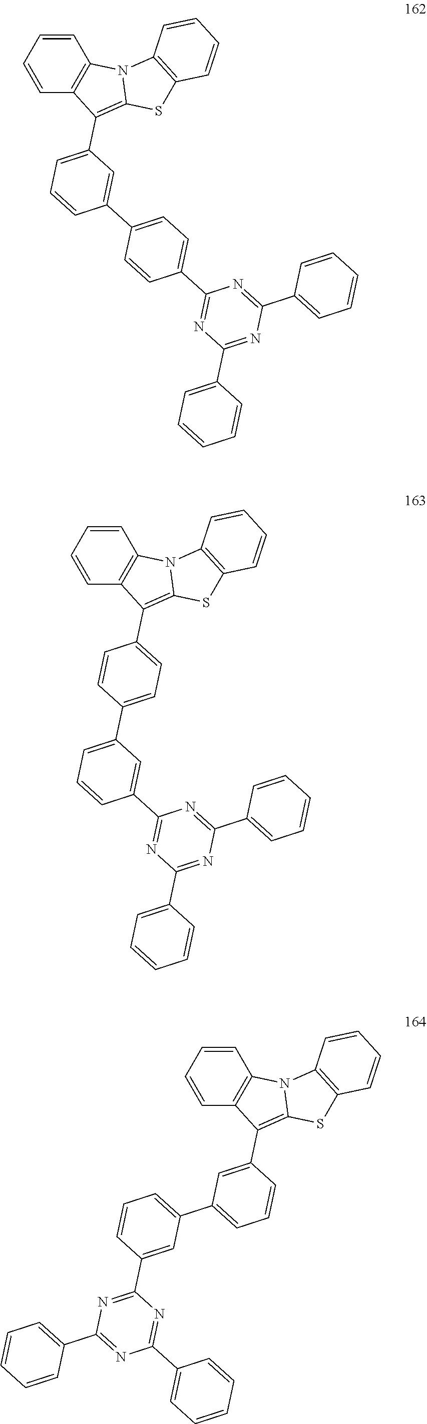 [SMILES: c1ccc(-c2nc(-c3ccccc3)nc(-c3ccc(-c4cccc(-c5c6ccccc6n6c5sc5ccccc56)c4)cc3)n2)cc1 ; c1ccc(-c2nc(-c3ccccc3)nc(-c3cccc(-c4ccc(-c5c6ccccc6n6c5sc5ccccc56)cc4)c3)n2)cc1 ; c1ccc(-c2nc(-c3ccccc3)nc(-c3cccc(-c4cccc(-c5c6ccccc6n6c5sc5ccccc56)c4)c3)n2)cc1]

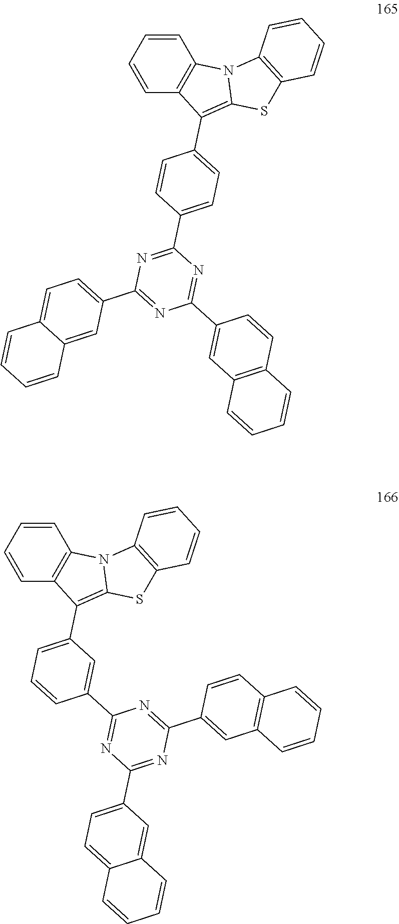 [SMILES: c1ccc2cc(-c3nc(-c4ccc(-c5c6ccccc6n6c5sc5ccccc56)cc4)nc(-c4ccc5ccccc5c4)n3)ccc2c1 ; c1cc(-c2nc(-c3ccc4ccccc4c3)nc(-c3ccc4ccccc4c3)n2)cc(-c2c3ccccc3n3c2sc2ccccc23)c1]

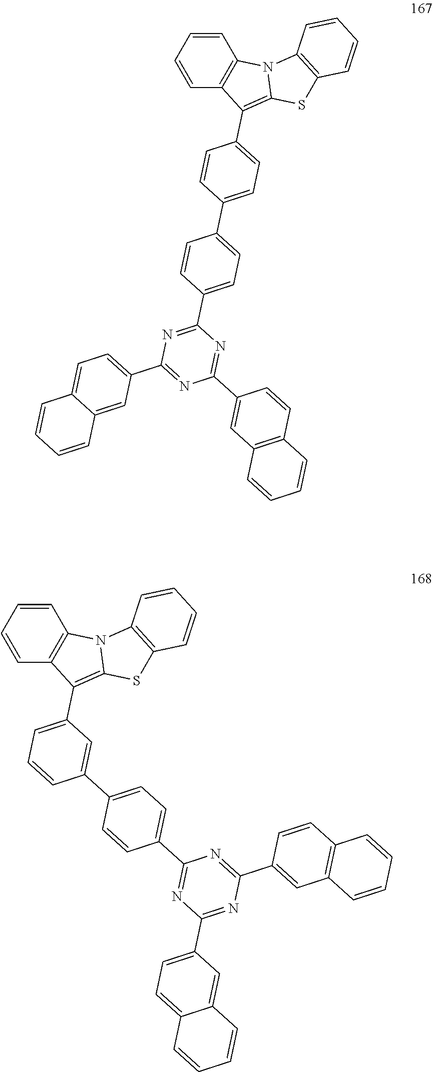 [SMILES: c1ccc2cc(-c3nc(-c4ccc(-c5ccc(-c6c7ccccc7n7c6sc6ccccc67)cc5)cc4)nc(-c4ccc5ccccc5c4)n3)ccc2c1 ; c1cc(-c2ccc(-c3nc(-c4ccc5ccccc5c4)nc(-c4ccc5ccccc5c4)n3)cc2)cc(-c2c3ccccc3n3c2sc2ccccc23)c1]

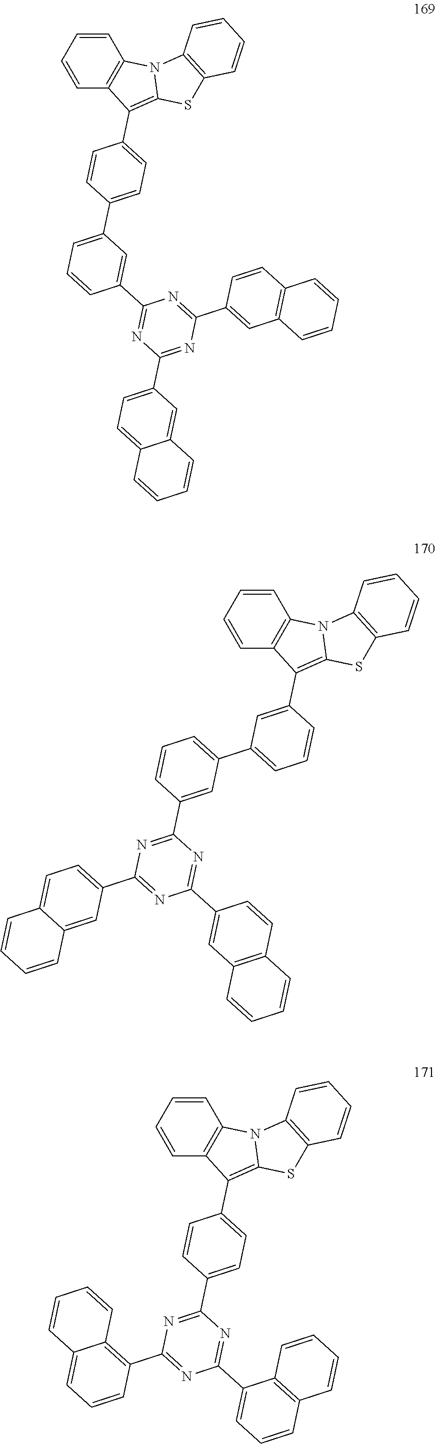 [SMILES: c1cc(-c2ccc(-c3c4ccccc4n4c3sc3ccccc34)cc2)cc(-c2nc(-c3ccc4ccccc4c3)nc(-c3ccc4ccccc4c3)n2)c1 ; c1cc(-c2cccc(-c3c4ccccc4n4c3sc3ccccc34)c2)cc(-c2nc(-c3ccc4ccccc4c3)nc(-c3ccc4ccccc4c3)n2)c1 ; c1ccc2c(-c3nc(-c4ccc(-c5c6ccccc6n6c5sc5ccccc56)cc4)nc(-c4cccc5ccccc45)n3)cccc2c1]

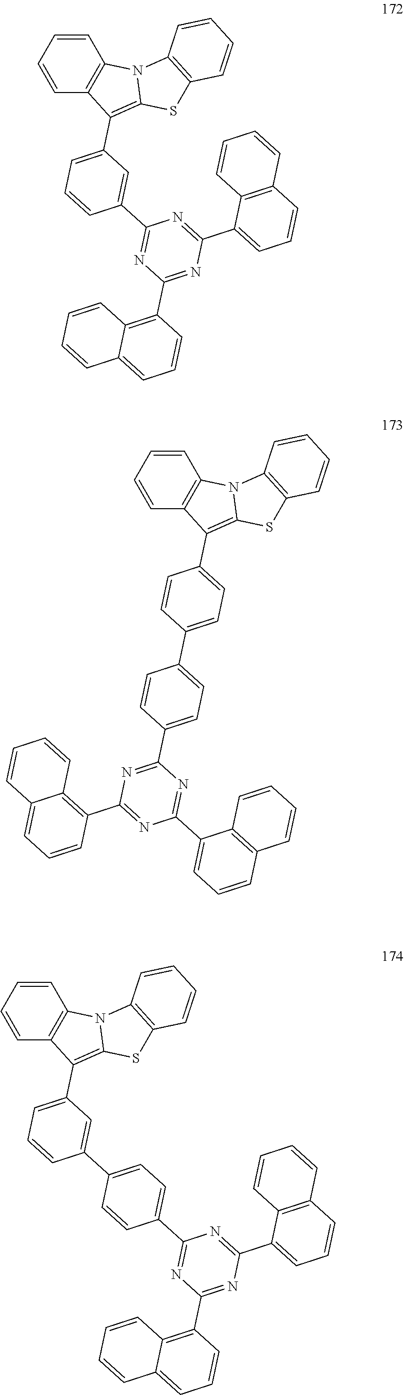 [SMILES: c1cc(-c2nc(-c3cccc4ccccc34)nc(-c3cccc4ccccc34)n2)cc(-c2c3ccccc3n3c2sc2ccccc23)c1 ; c1ccc2c(-c3nc(-c4ccc(-c5ccc(-c6c7ccccc7n7c6sc6ccccc67)cc5)cc4)nc(-c4cccc5ccccc45)n3)cccc2c1 ; c1cc(-c2ccc(-c3nc(-c4cccc5ccccc45)nc(-c4cccc5ccccc45)n3)cc2)cc(-c2c3ccccc3n3c2sc2ccccc23)c1]

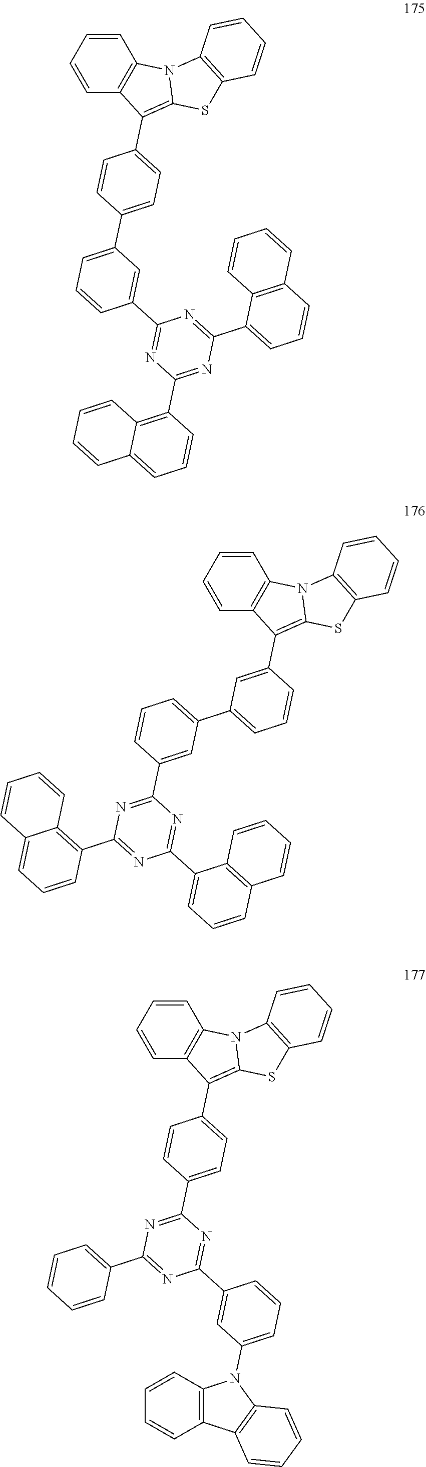 [SMILES: c1cc(-c2ccc(-c3c4ccccc4n4c3sc3ccccc34)cc2)cc(-c2nc(-c3cccc4ccccc34)nc(-c3cccc4ccccc34)n2)c1 ; c1cc(-c2cccc(-c3c4ccccc4n4c3sc3ccccc34)c2)cc(-c2nc(-c3cccc4ccccc34)nc(-c3cccc4ccccc34)n2)c1 ; c1ccc(-c2nc(-c3ccc(-c4c5ccccc5n5c4sc4ccccc45)cc3)nc(-c3cccc(-n4c5ccccc5c5ccccc54)c3)n2)cc1]

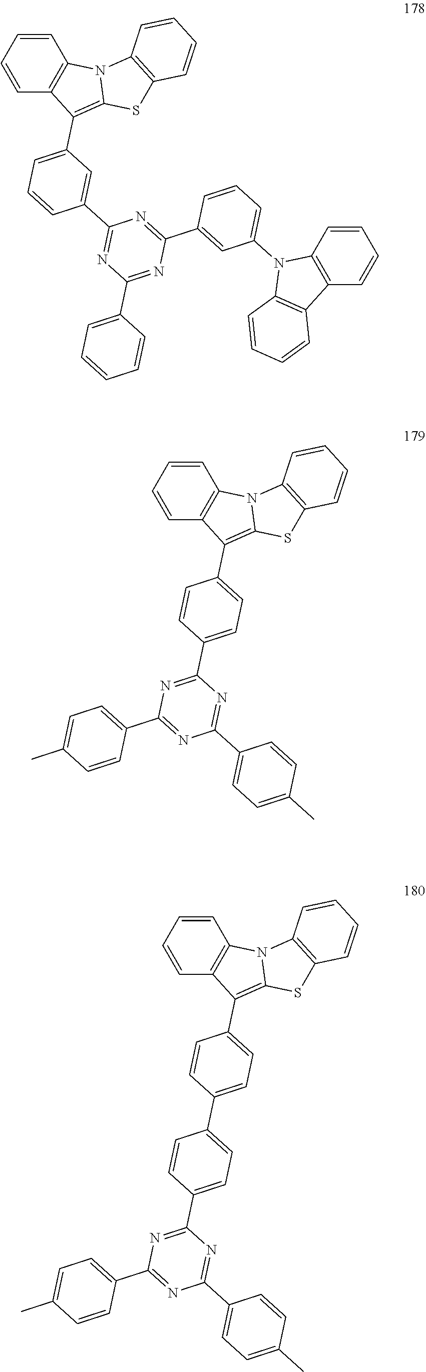 [SMILES: c1ccc(-c2nc(-c3cccc(-c4c5ccccc5n5c4sc4ccccc45)c3)nc(-c3cccc(-n4c5ccccc5c5ccccc54)c3)n2)cc1 ; Cc1ccc(-c2nc(-c3ccc(C)cc3)nc(-c3ccc(-c4c5ccccc5n5c4sc4ccccc45)cc3)n2)cc1 ; Cc1ccc(-c2nc(-c3ccc(C)cc3)nc(-c3ccc(-c4ccc(-c5c6ccccc6n6c5sc5ccccc56)cc4)cc3)n2)cc1]

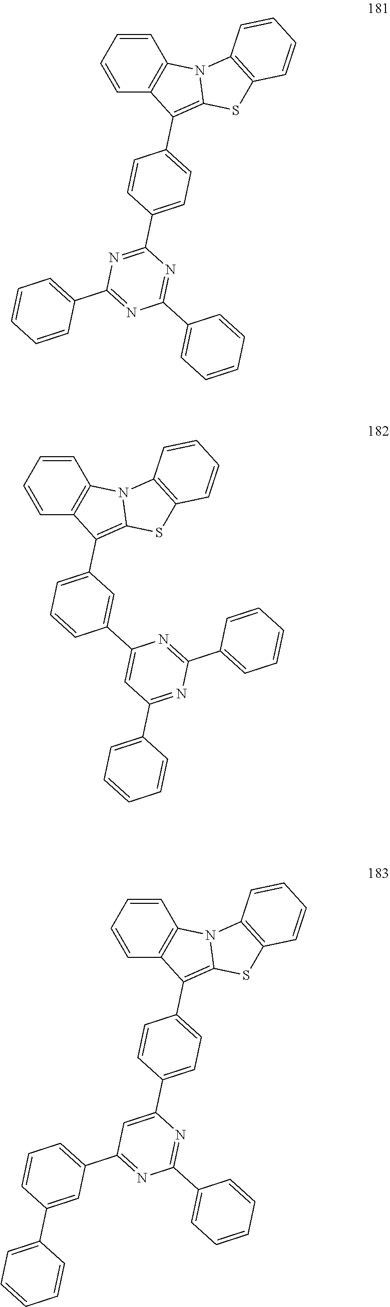 [SMILES: c1ccc(-c2nc(-c3ccccc3)nc(-c3ccc(-c4c5ccccc5n5c4sc4ccccc45)cc3)n2)cc1 ; c1ccc(-c2cc(-c3cccc(-c4c5ccccc5n5c4sc4ccccc45)c3)nc(-c3ccccc3)n2)cc1 ; c1ccc(-c2cccc(-c3cc(-c4ccc(-c5c6ccccc6n6c5sc5ccccc56)cc4)nc(-c4ccccc4)n3)c2)cc1]

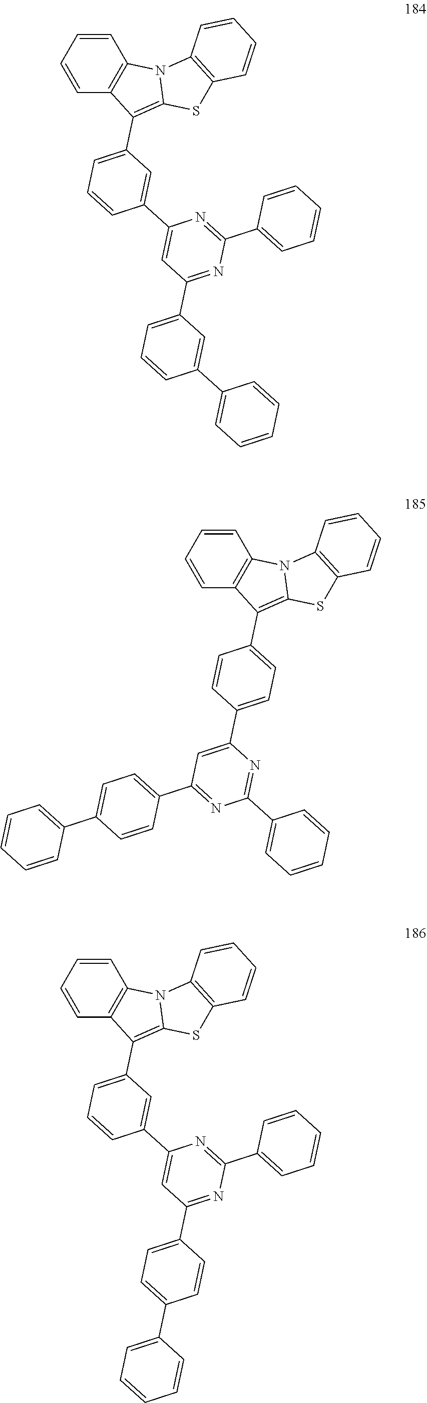 [SMILES: c1ccc(-c2cccc(-c3cc(-c4cccc(-c5c6ccccc6n6c5sc5ccccc56)c4)nc(-c4ccccc4)n3)c2)cc1 ; c1ccc(-c2ccc(-c3cc(-c4ccc(-c5c6ccccc6n6c5sc5ccccc56)cc4)nc(-c4ccccc4)n3)cc2)cc1 ; c1ccc(-c2ccc(-c3cc(-c4cccc(-c5c6ccccc6n6c5sc5ccccc56)c4)nc(-c4ccccc4)n3)cc2)cc1]

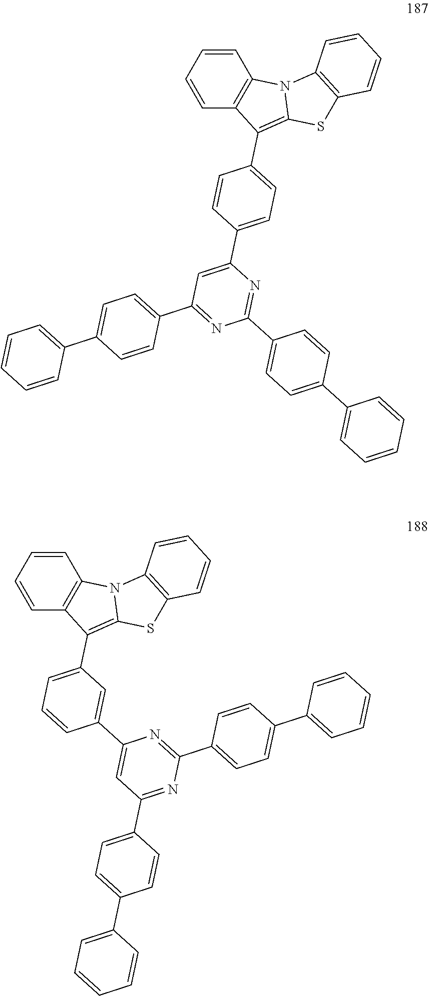 [SMILES: c1ccc(-c2ccc(-c3cc(-c4ccc(-c5c6ccccc6n6c5sc5ccccc56)cc4)nc(-c4ccc(-c5ccccc5)cc4)n3)cc2)cc1 ; c1ccc(-c2ccc(-c3cc(-c4cccc(-c5c6ccccc6n6c5sc5ccccc56)c4)nc(-c4ccc(-c5ccccc5)cc4)n3)cc2)cc1]

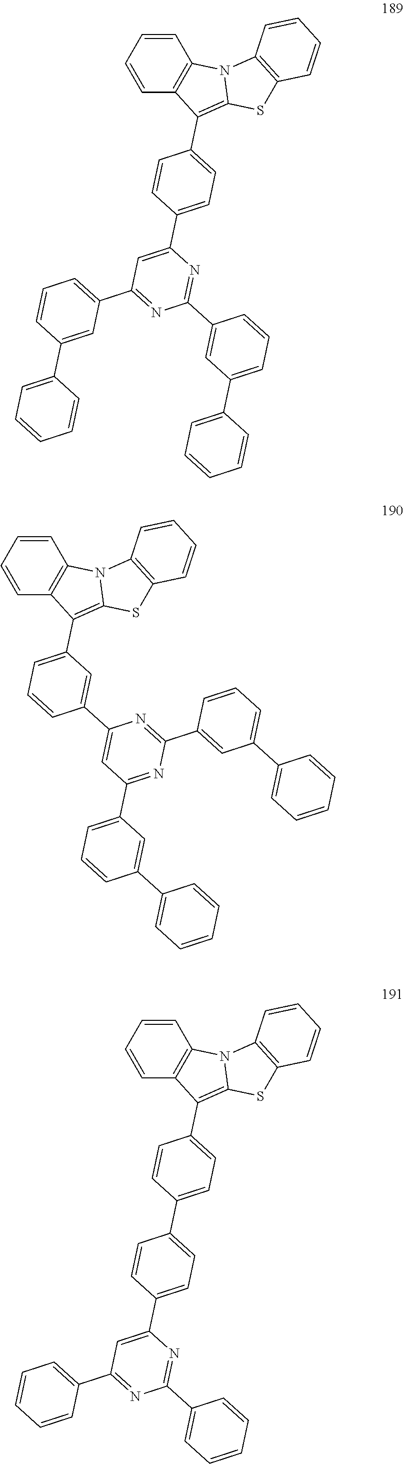 [SMILES: c1ccc(-c2cccc(-c3cc(-c4ccc(-c5c6ccccc6n6c5sc5ccccc56)cc4)nc(-c4cccc(-c5ccccc5)c4)n3)c2)cc1 ; c1ccc(-c2cccc(-c3cc(-c4cccc(-c5c6ccccc6n6c5sc5ccccc56)c4)nc(-c4cccc(-c5ccccc5)c4)n3)c2)cc1 ; c1ccc(-c2cc(-c3ccc(-c4ccc(-c5c6ccccc6n6c5sc5ccccc56)cc4)cc3)nc(-c3ccccc3)n2)cc1]

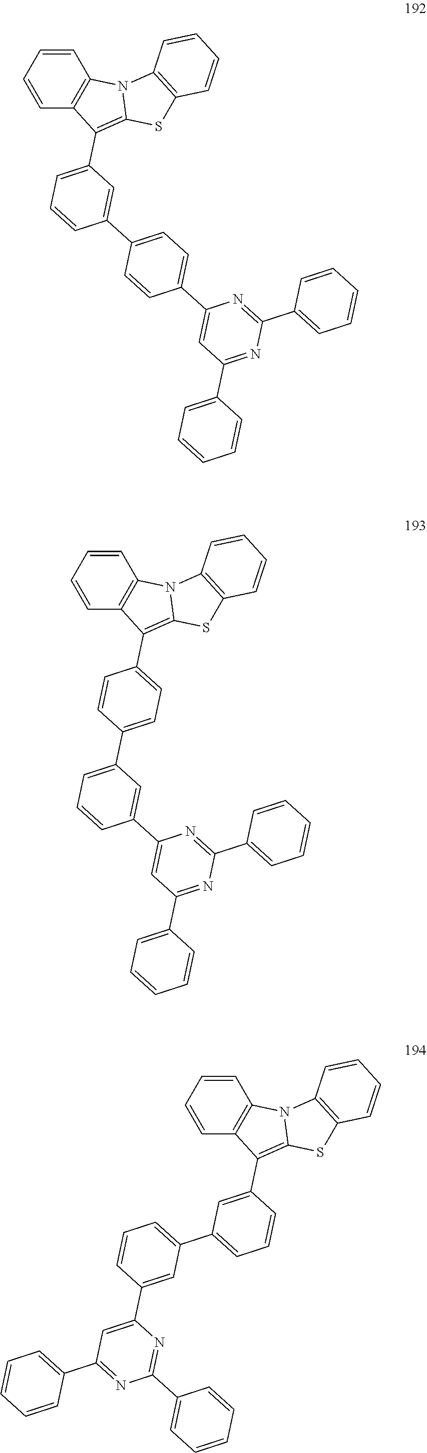 [SMILES: c1ccc(-c2cc(-c3ccc(-c4cccc(-c5c6ccccc6n6c5sc5ccccc56)c4)cc3)nc(-c3ccccc3)n2)cc1 ; c1ccc(-c2cc(-c3cccc(-c4ccc(-c5c6ccccc6n6c5sc5ccccc56)cc4)c3)nc(-c3ccccc3)n2)cc1 ; c1ccc(-c2cc(-c3cccc(-c4cccc(-c5c6ccccc6n6c5sc5ccccc56)c4)c3)nc(-c3ccccc3)n2)cc1]

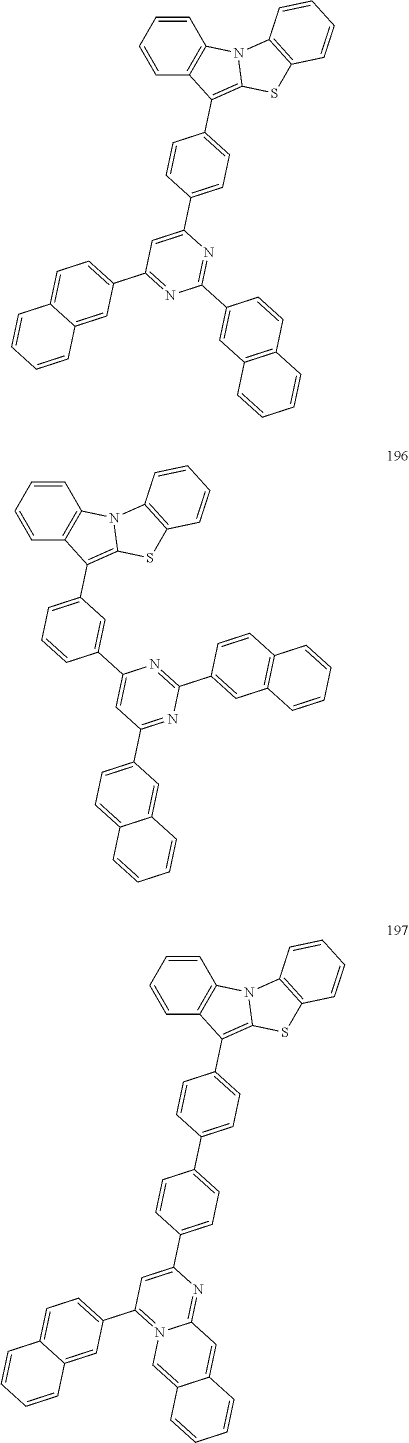

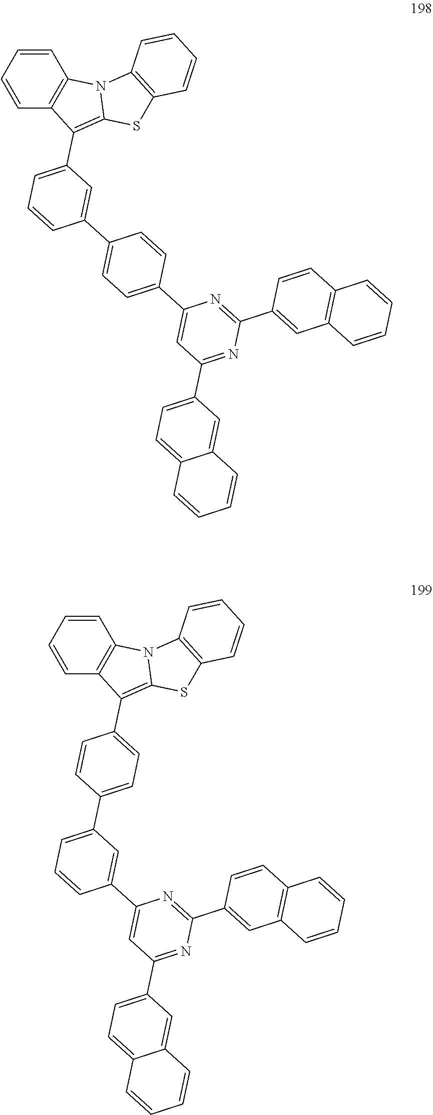 [SMILES: c1cc(-c2ccc(-c3cc(-c4ccc5ccccc5c4)nc(-c4ccc5ccccc5c4)n3)cc2)cc(-c2c3ccccc3n3c2sc2ccccc23)c1 ; c1cc(-c2ccc(-c3c4ccccc4n4c3sc3ccccc34)cc2)cc(-c2cc(-c3ccc4ccccc4c3)nc(-c3ccc4ccccc4c3)n2)c1]

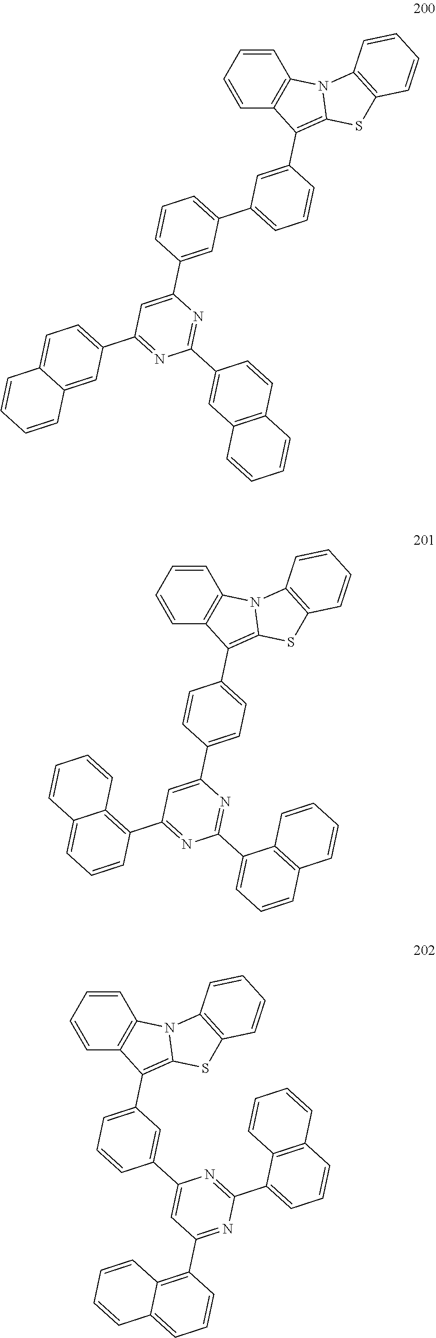 [SMILES: c1cc(-c2cccc(-c3c4ccccc4n4c3sc3ccccc34)c2)cc(-c2cc(-c3ccc4ccccc4c3)nc(-c3ccc4ccccc4c3)n2)c1 ; c1ccc2c(-c3cc(-c4ccc(-c5c6ccccc6n6c5sc5ccccc56)cc4)nc(-c4cccc5ccccc45)n3)cccc2c1 ; c1cc(-c2cc(-c3cccc4ccccc34)nc(-c3cccc4ccccc34)n2)cc(-c2c3ccccc3n3c2sc2ccccc23)c1]

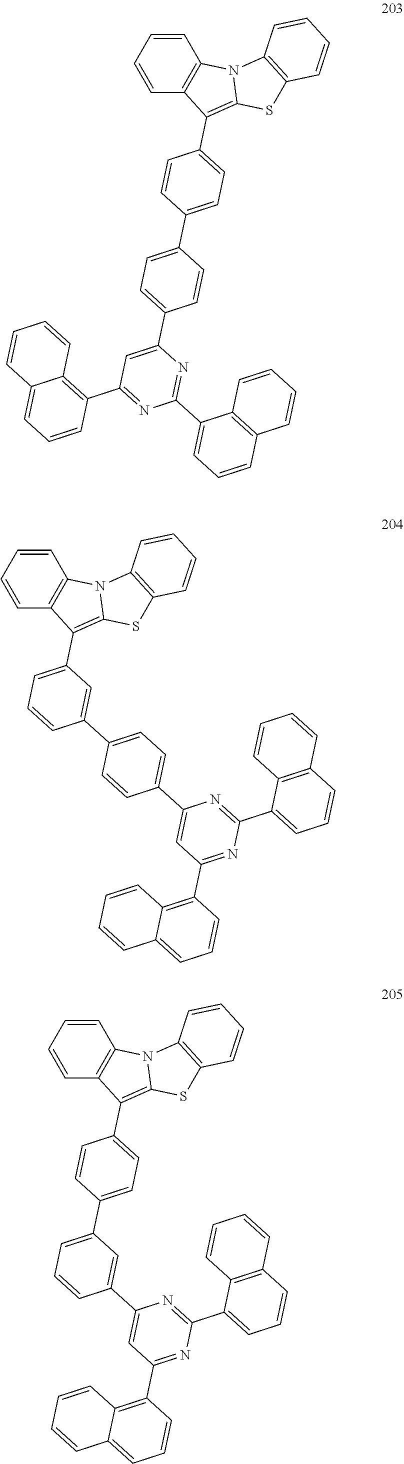 [SMILES: c1ccc2c(-c3cc(-c4ccc(-c5ccc(-c6c7ccccc7n7c6sc6ccccc67)cc5)cc4)nc(-c4cccc5ccccc45)n3)cccc2c1 ; c1cc(-c2ccc(-c3cc(-c4cccc5ccccc45)nc(-c4cccc5ccccc45)n3)cc2)cc(-c2c3ccccc3n3c2sc2ccccc23)c1 ; c1cc(-c2ccc(-c3c4ccccc4n4c3sc3ccccc34)cc2)cc(-c2cc(-c3cccc4ccccc34)nc(-c3cccc4ccccc34)n2)c1]

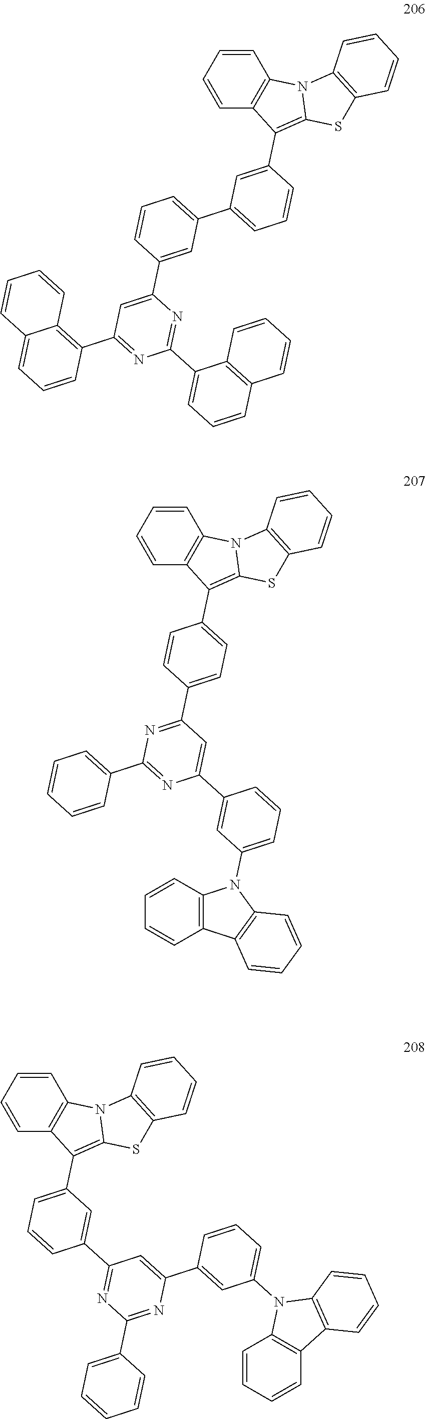 [SMILES: c1cc(-c2cccc(-c3c4ccccc4n4c3sc3ccccc34)c2)cc(-c2cc(-c3cccc4ccccc34)nc(-c3cccc4ccccc34)n2)c1 ; c1ccc(-c2nc(-c3ccc(-c4c5ccccc5n5c4sc4ccccc45)cc3)cc(-c3cccc(-n4c5ccccc5c5ccccc54)c3)n2)cc1 ; c1ccc(-c2nc(-c3cccc(-c4c5ccccc5n5c4sc4ccccc45)c3)cc(-c3cccc(-n4c5ccccc5c5ccccc54)c3)n2)cc1]

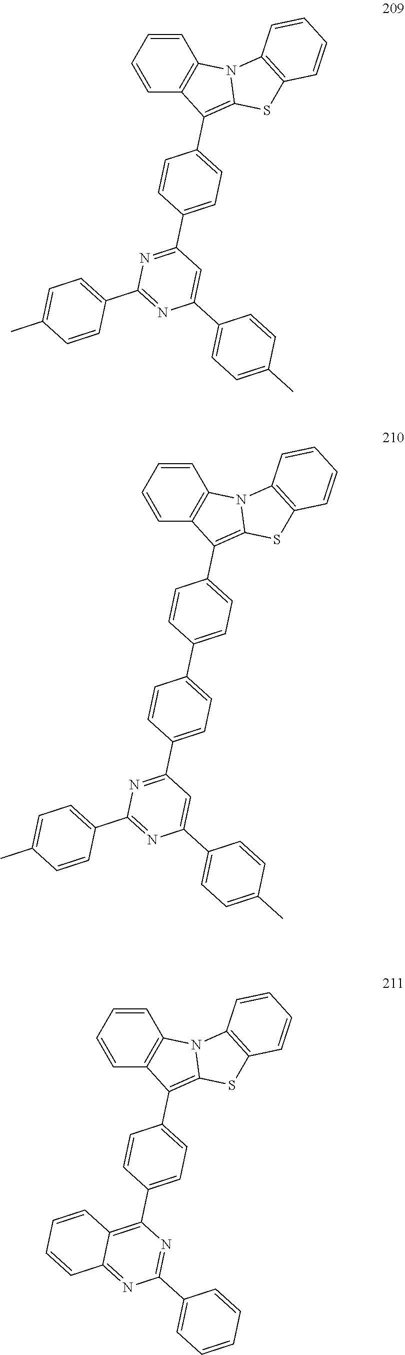 [SMILES: Cc1ccc(-c2cc(-c3ccc(-c4c5ccccc5n5c4sc4ccccc45)cc3)nc(-c3ccc(C)cc3)n2)cc1 ; Cc1ccc(-c2cc(-c3ccc(-c4ccc(-c5c6ccccc6n6c5sc5ccccc56)cc4)cc3)nc(-c3ccc(C)cc3)n2)cc1 ; c1ccc(-c2nc(-c3ccc(-c4c5ccccc5n5c4sc4ccccc45)cc3)c3ccccc3n2)cc1]

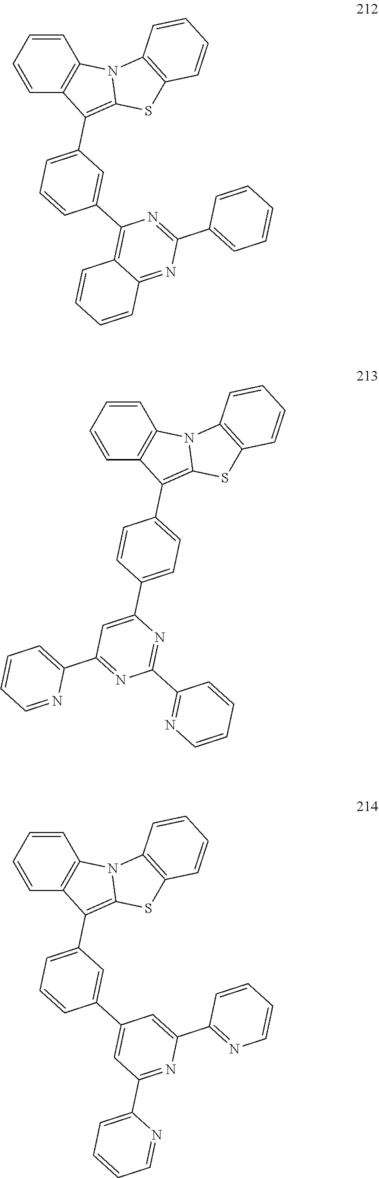 [SMILES: c1ccc(-c2nc(-c3cccc(-c4c5ccccc5n5c4sc4ccccc45)c3)c3ccccc3n2)cc1 ; c1ccc(-c2cc(-c3ccc(-c4c5ccccc5n5c4sc4ccccc45)cc3)nc(-c3ccccn3)n2)nc1 ; c1ccc(-c2cc(-c3cccc(-c4c5ccccc5n5c4sc4ccccc45)c3)cc(-c3ccccn3)n2)nc1]

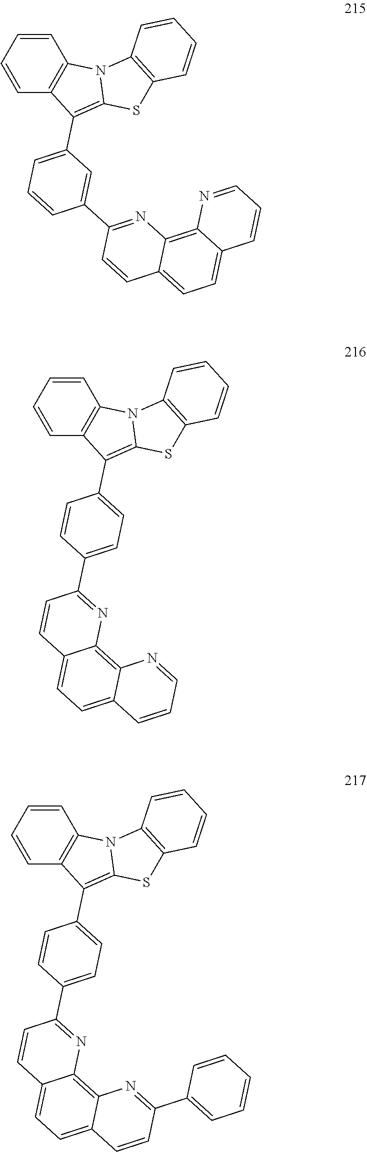 [SMILES: c1cc(-c2ccc3ccc4cccnc4c3n2)cc(-c2c3ccccc3n3c2sc2ccccc23)c1 ; c1cnc2c(c1)ccc1ccc(-c3ccc(-c4c5ccccc5n5c4sc4ccccc45)cc3)nc12 ; c1ccc(-c2ccc3ccc4ccc(-c5ccc(-c6c7ccccc7n7c6sc6ccccc67)cc5)nc4c3n2)cc1]

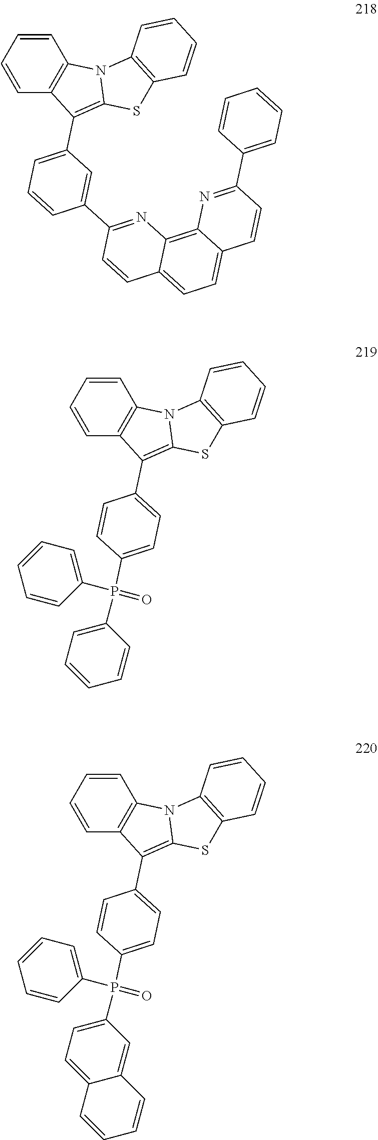 [SMILES: c1ccc(-c2ccc3ccc4ccc(-c5cccc(-c6c7ccccc7n7c6sc6ccccc67)c5)nc4c3n2)cc1 ; O=P(c1ccccc1)(c1ccccc1)c1ccc(-c2c3ccccc3n3c2sc2ccccc23)cc1 ; O=P(c1ccccc1)(c1ccc(-c2c3ccccc3n3c2sc2ccccc23)cc1)c1ccc2ccccc2c1]

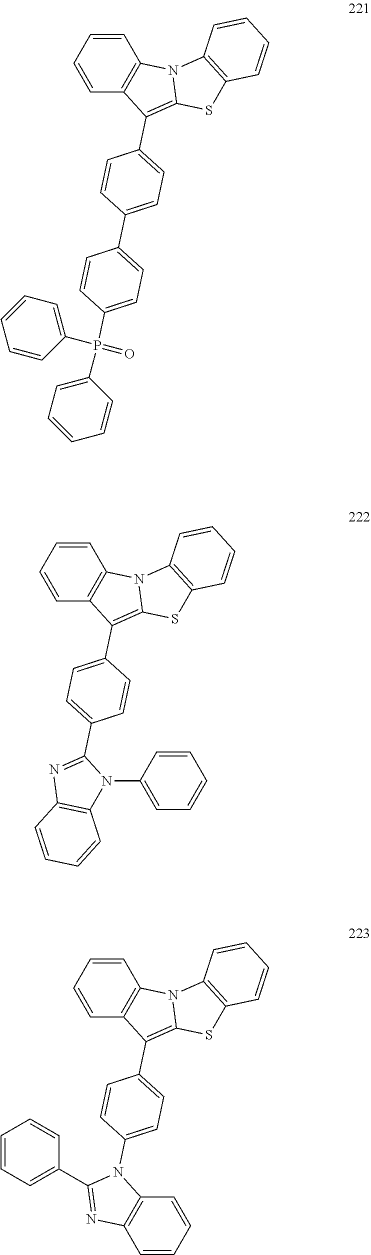 [SMILES: O=P(c1ccccc1)(c1ccccc1)c1ccc(-c2ccc(-c3c4ccccc4n4c3sc3ccccc34)cc2)cc1 ; c1ccc(-n2c(-c3ccc(-c4c5ccccc5n5c4sc4ccccc45)cc3)nc3ccccc32)cc1 ; c1ccc(-c2nc3ccccc3n2-c2ccc(-c3c4ccccc4n4c3sc3ccccc34)cc2)cc1]

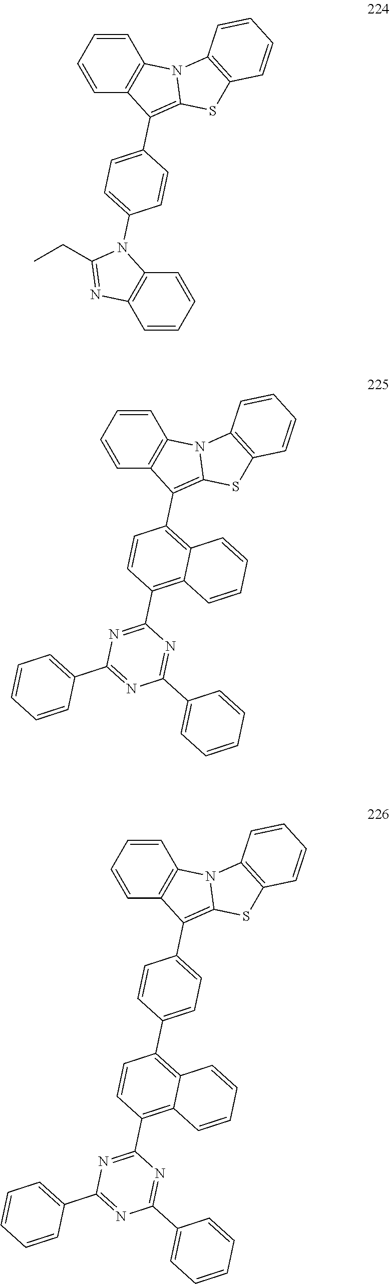 [SMILES: CCc1nc2ccccc2n1-c1ccc(-c2c3ccccc3n3c2sc2ccccc23)cc1 ; c1ccc(-c2nc(-c3ccccc3)nc(-c3ccc(-c4c5ccccc5n5c4sc4ccccc45)c4ccccc34)n2)cc1 ; c1ccc(-c2nc(-c3ccccc3)nc(-c3ccc(-c4ccc(-c5c6ccccc6n6c5sc5ccccc56)cc4)c4ccccc34)n2)cc1]

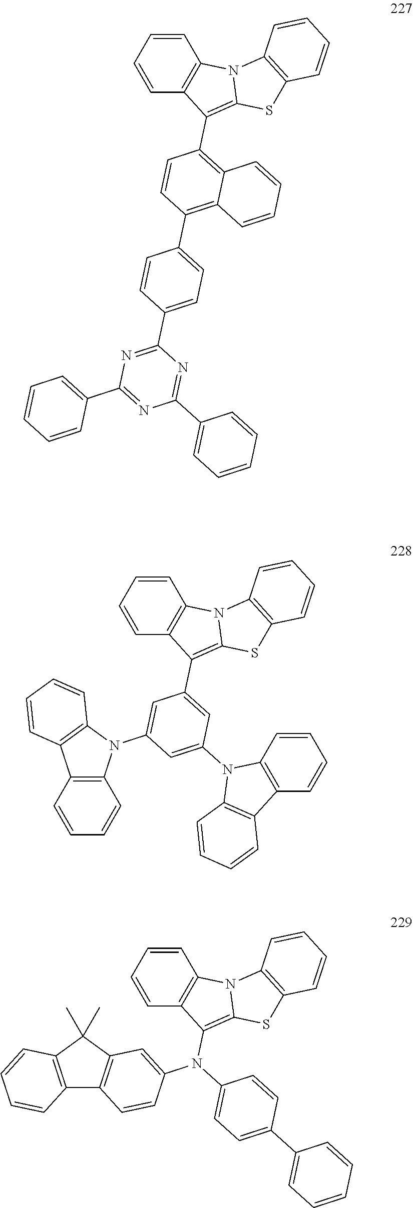 [SMILES: c1ccc(-c2nc(-c3ccccc3)nc(-c3ccc(-c4ccc(-c5c6ccccc6n6c5sc5ccccc56)c5ccccc45)cc3)n2)cc1 ; c1ccc2c(c1)sc1c(-c3cc(-n4c5ccccc5c5ccccc54)cc(-n4c5ccccc5c5ccccc54)c3)c3ccccc3n12 ; CC1(C)c2ccccc2-c2ccc(N(c3ccc(-c4ccccc4)cc3)c3c4ccccc4n4c3sc3ccccc34)cc21]

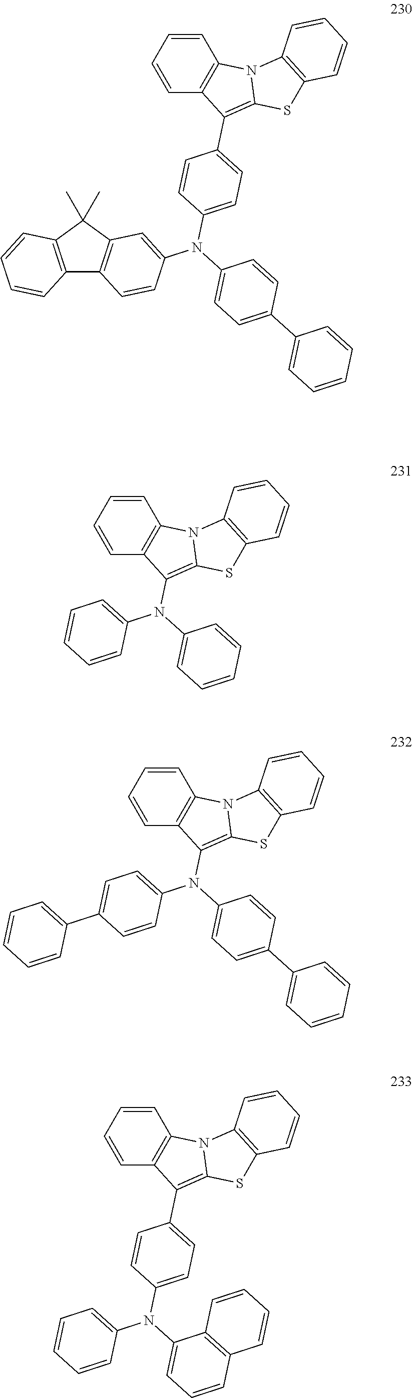 [SMILES: CC1(C)c2ccccc2-c2ccc(N(c3ccc(-c4ccccc4)cc3)c3ccc(-c4c5ccccc5n5c4sc4ccccc45)cc3)cc21 ; c1ccc(N(c2ccccc2)c2c3ccccc3n3c2sc2ccccc23)cc1 ; c1ccc(-c2ccc(N(c3ccc(-c4ccccc4)cc3)c3c4ccccc4n4c3sc3ccccc34)cc2)cc1 ; c1ccc(N(c2ccc(-c3c4ccccc4n4c3sc3ccccc34)cc2)c2cccc3ccccc23)cc1]

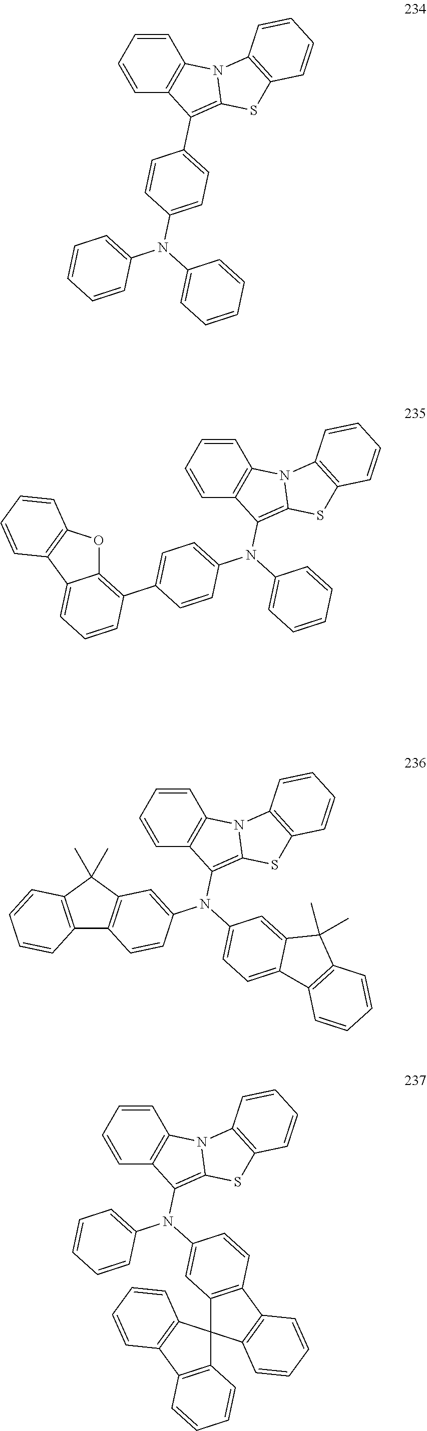 [SMILES: c1ccc(N(c2ccccc2)c2ccc(-c3c4ccccc4n4c3sc3ccccc34)cc2)cc1 ; c1ccc(N(c2ccc(-c3cccc4c3oc3ccccc34)cc2)c2c3ccccc3n3c2sc2ccccc23)cc1 ; CC1(C)c2ccccc2-c2ccc(N(c3ccc4c(c3)C(C)(C)c3ccccc3-4)c3c4ccccc4n4c3sc3ccccc34)cc21 ; c1ccc(N(c2ccc3c(c2)C2(c4ccccc4-c4ccccc42)c2ccccc2-3)c2c3ccccc3n3c2sc2ccccc23)cc1]

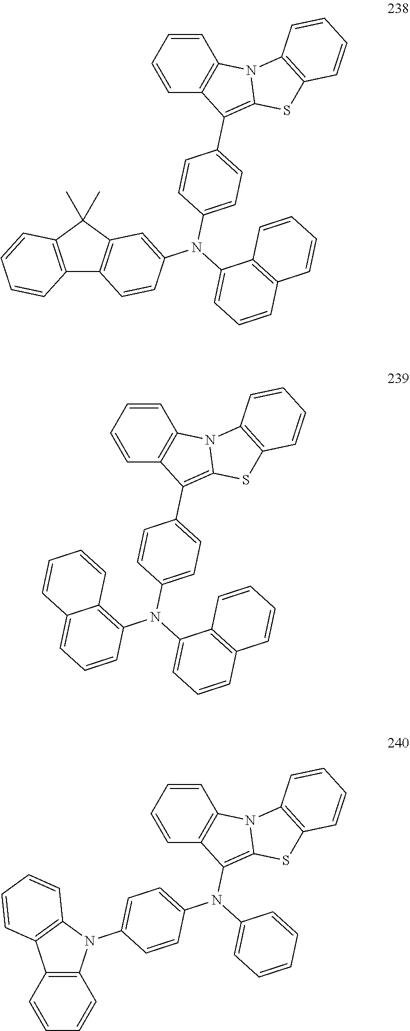 [SMILES: CC1(C)c2ccccc2-c2ccc(N(c3ccc(-c4c5ccccc5n5c4sc4ccccc45)cc3)c3cccc4ccccc34)cc21 ; c1ccc2c(N(c3ccc(-c4c5ccccc5n5c4sc4ccccc45)cc3)c3cccc4ccccc34)cccc2c1 ; c1ccc(N(c2ccc(-n3c4ccccc4c4ccccc43)cc2)c2c3ccccc3n3c2sc2ccccc23)cc1]

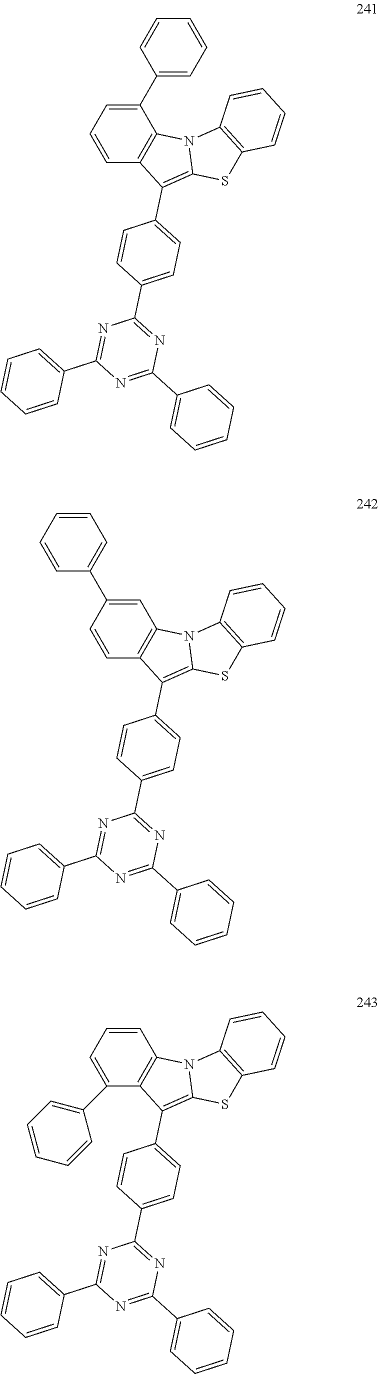 [SMILES: c1ccc(-c2nc(-c3ccccc3)nc(-c3ccc(-c4c5cccc(-c6ccccc6)c5n5c4sc4ccccc45)cc3)n2)cc1 ; c1ccc(-c2ccc3c(-c4ccc(-c5nc(-c6ccccc6)nc(-c6ccccc6)n5)cc4)c4sc5ccccc5n4c3c2)cc1 ; c1ccc(-c2nc(-c3ccccc3)nc(-c3ccc(-c4c5c(-c6ccccc6)cccc5n5c4sc4ccccc45)cc3)n2)cc1]

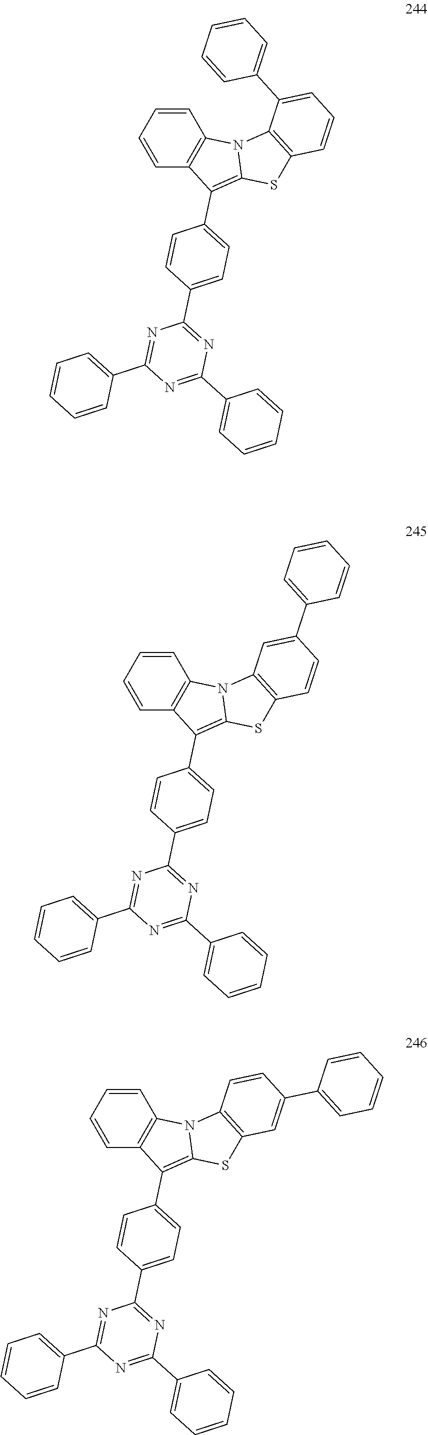 [SMILES: c1ccc(-c2nc(-c3ccccc3)nc(-c3ccc(-c4c5ccccc5n5c4sc4cccc(-c6ccccc6)c45)cc3)n2)cc1 ; c1ccc(-c2ccc3sc4c(-c5ccc(-c6nc(-c7ccccc7)nc(-c7ccccc7)n6)cc5)c5ccccc5n4c3c2)cc1 ; c1ccc(-c2ccc3c(c2)sc2c(-c4ccc(-c5nc(-c6ccccc6)nc(-c6ccccc6)n5)cc4)c4ccccc4n23)cc1]

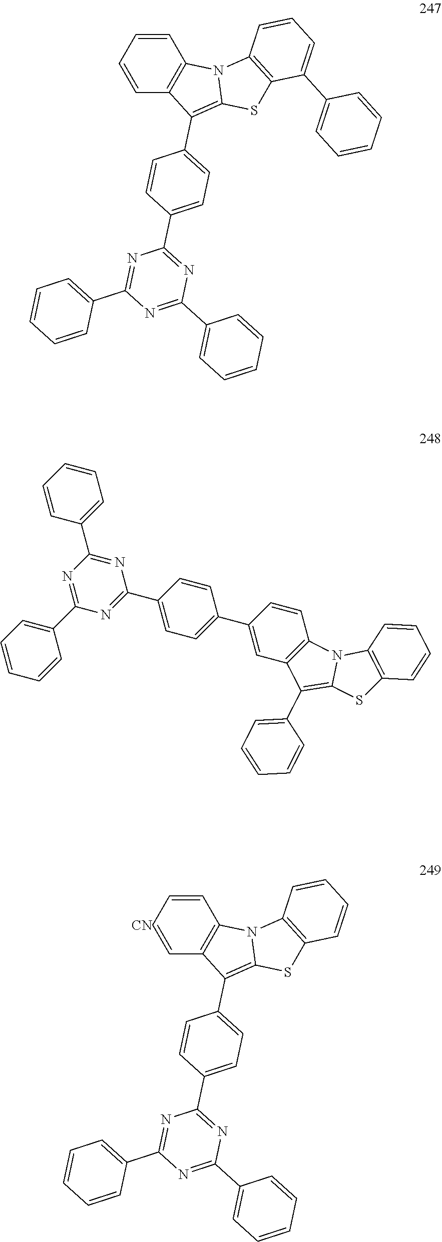

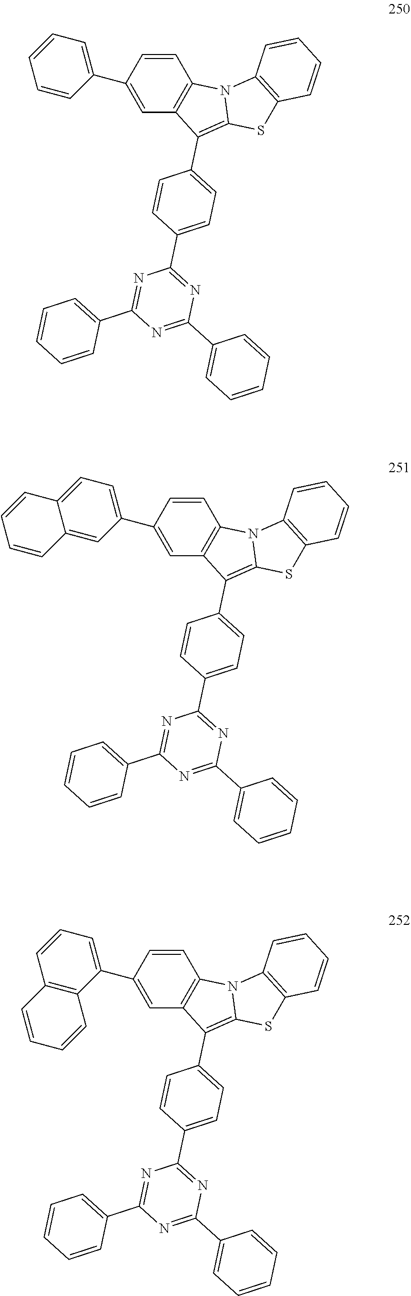 [SMILES: c1ccc(-c2ccc3c(c2)c(-c2ccc(-c4nc(-c5ccccc5)nc(-c5ccccc5)n4)cc2)c2sc4ccccc4n23)cc1 ; c1ccc(-c2nc(-c3ccccc3)nc(-c3ccc(-c4c5cc(-c6ccc7ccccc7c6)ccc5n5c4sc4ccccc45)cc3)n2)cc1 ; c1ccc(-c2nc(-c3ccccc3)nc(-c3ccc(-c4c5cc(-c6cccc7ccccc67)ccc5n5c4sc4ccccc45)cc3)n2)cc1]

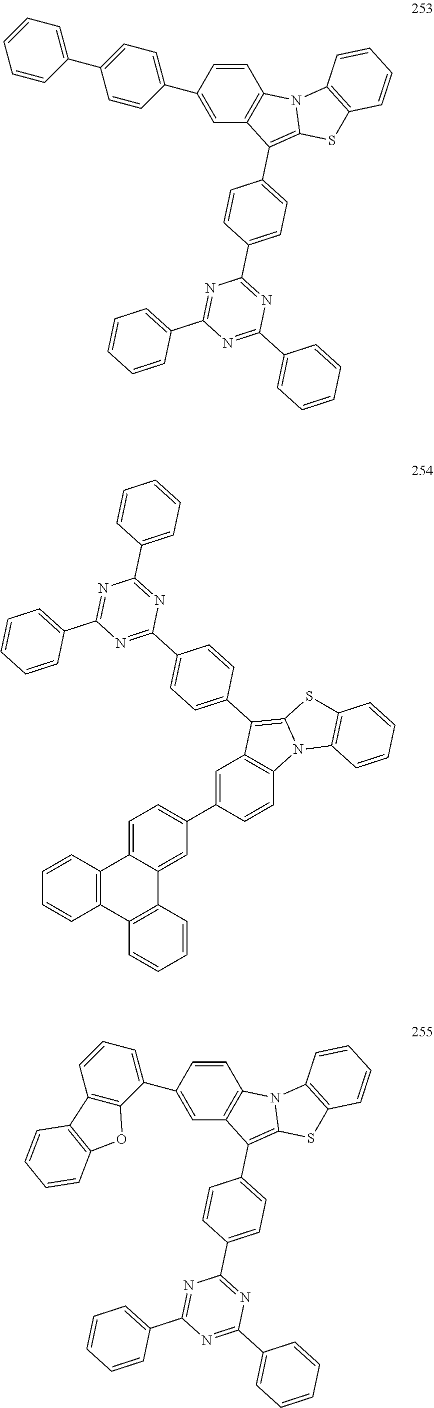 [SMILES: c1ccc(-c2ccc(-c3ccc4c(c3)c(-c3ccc(-c5nc(-c6ccccc6)nc(-c6ccccc6)n5)cc3)c3sc5ccccc5n34)cc2)cc1 ; c1ccc(-c2nc(-c3ccccc3)nc(-c3ccc(-c4c5cc(-c6ccc7c8ccccc8c8ccccc8c7c6)ccc5n5c4sc4ccccc45)cc3)n2)cc1 ; c1ccc(-c2nc(-c3ccccc3)nc(-c3ccc(-c4c5cc(-c6cccc7c6oc6ccccc67)ccc5n5c4sc4ccccc45)cc3)n2)cc1]

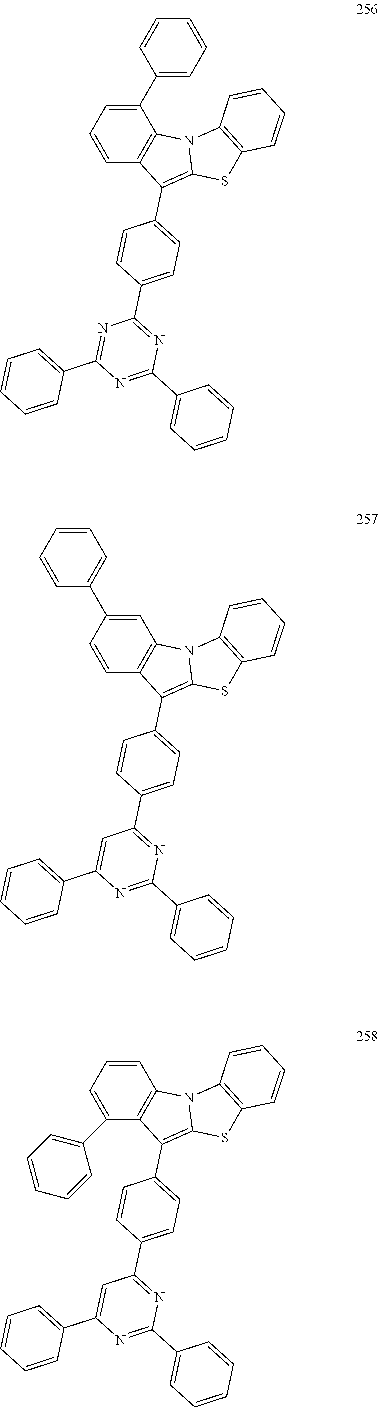 [SMILES: c1ccc(-c2nc(-c3ccccc3)nc(-c3ccc(-c4c5cccc(-c6ccccc6)c5n5c4sc4ccccc45)cc3)n2)cc1 ; c1ccc(-c2ccc3c(-c4ccc(-c5cc(-c6ccccc6)nc(-c6ccccc6)n5)cc4)c4sc5ccccc5n4c3c2)cc1 ; c1ccc(-c2cc(-c3ccc(-c4c5c(-c6ccccc6)cccc5n5c4sc4ccccc45)cc3)nc(-c3ccccc3)n2)cc1]

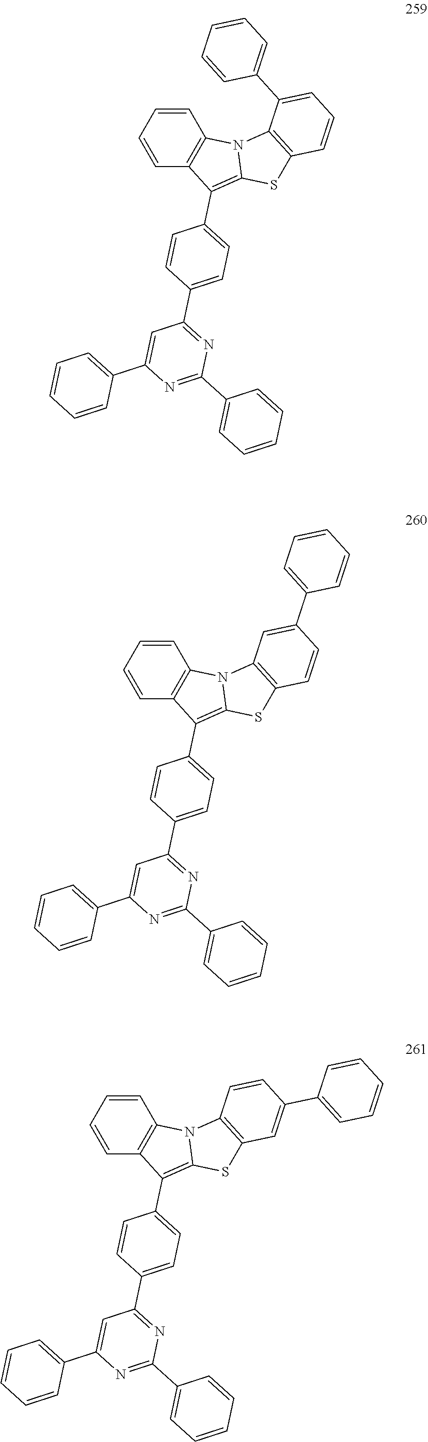 [SMILES: c1ccc(-c2cc(-c3ccc(-c4c5ccccc5n5c4sc4cccc(-c6ccccc6)c45)cc3)nc(-c3ccccc3)n2)cc1 ; c1ccc(-c2ccc3sc4c(-c5ccc(-c6cc(-c7ccccc7)nc(-c7ccccc7)n6)cc5)c5ccccc5n4c3c2)cc1 ; c1ccc(-c2ccc3c(c2)sc2c(-c4ccc(-c5cc(-c6ccccc6)nc(-c6ccccc6)n5)cc4)c4ccccc4n23)cc1]

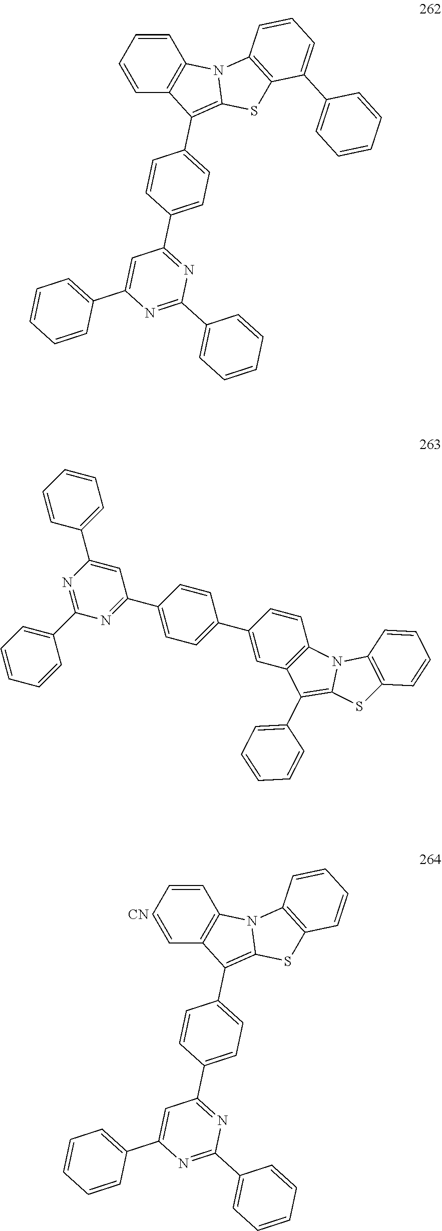

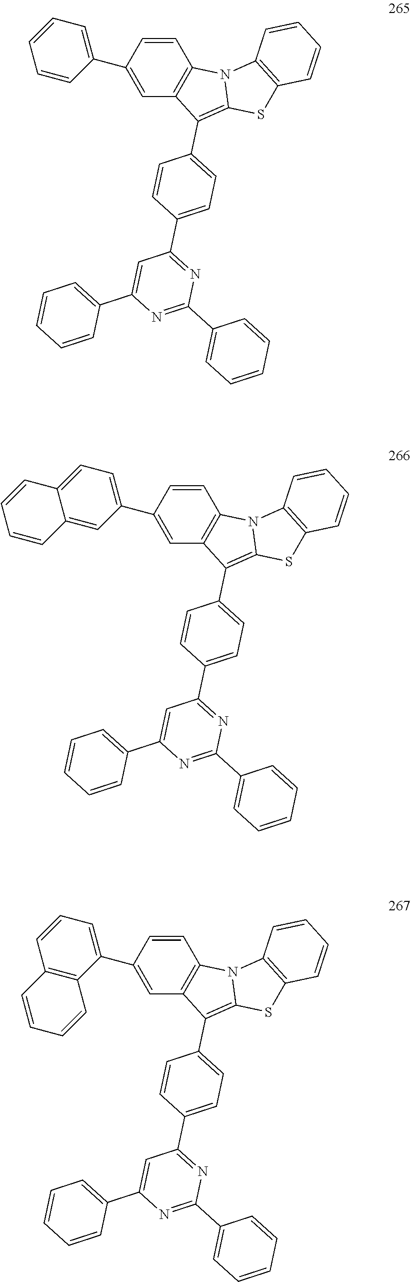 [SMILES: c1ccc(-c2ccc3c(c2)c(-c2ccc(-c4cc(-c5ccccc5)nc(-c5ccccc5)n4)cc2)c2sc4ccccc4n23)cc1 ; c1ccc(-c2cc(-c3ccc(-c4c5cc(-c6ccc7ccccc7c6)ccc5n5c4sc4ccccc45)cc3)nc(-c3ccccc3)n2)cc1 ; c1ccc(-c2cc(-c3ccc(-c4c5cc(-c6cccc7ccccc67)ccc5n5c4sc4ccccc45)cc3)nc(-c3ccccc3)n2)cc1]

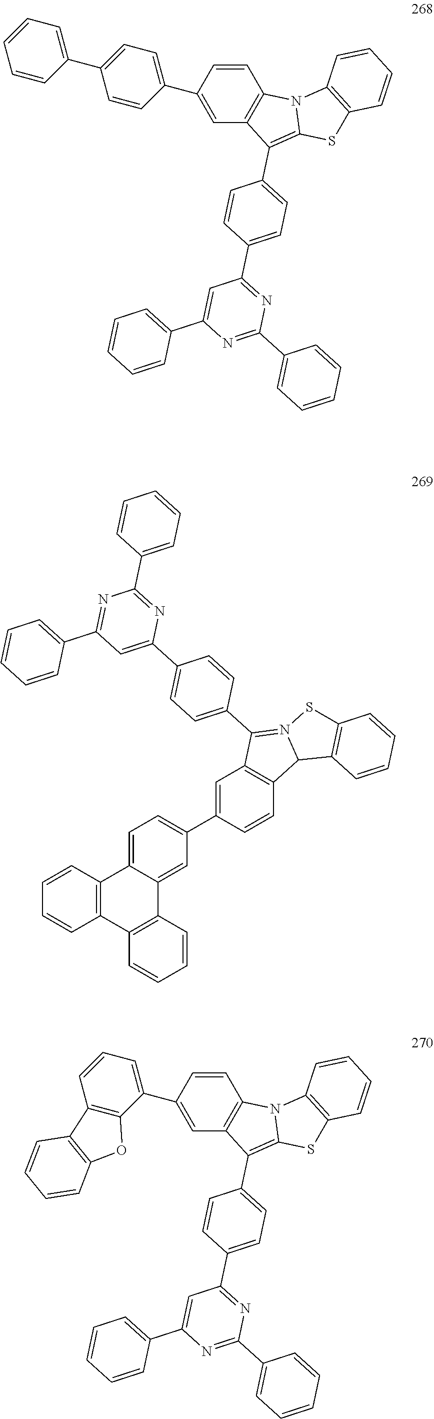

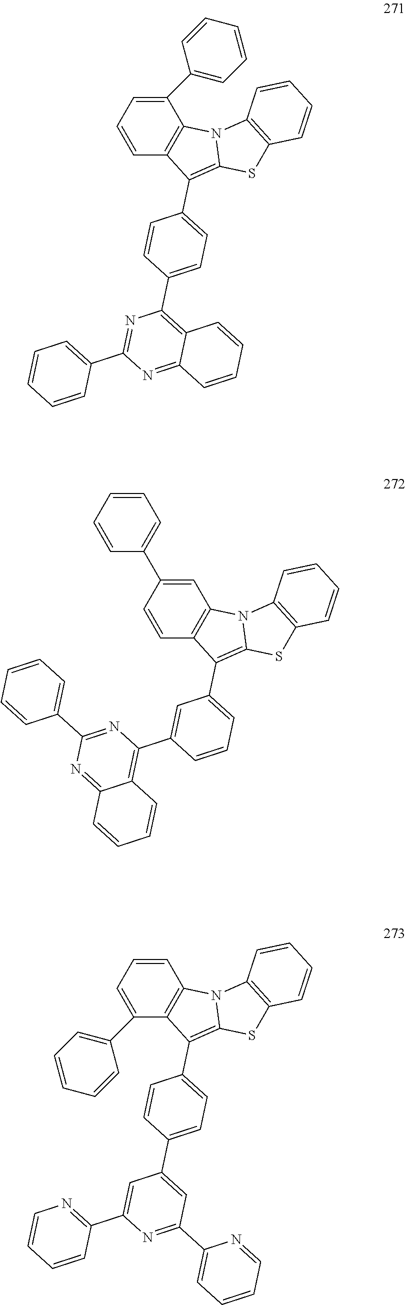 [SMILES: c1ccc(-c2nc(-c3ccc(-c4c5cccc(-c6ccccc6)c5n5c4sc4ccccc45)cc3)c3ccccc3n2)cc1 ; c1ccc(-c2ccc3c(-c4cccc(-c5nc(-c6ccccc6)nc6ccccc56)c4)c4sc5ccccc5n4c3c2)cc1 ; c1ccc(-c2cccc3c2c(-c2ccc(-c4cc(-c5ccccn5)nc(-c5ccccn5)c4)cc2)c2sc4ccccc4n23)cc1]

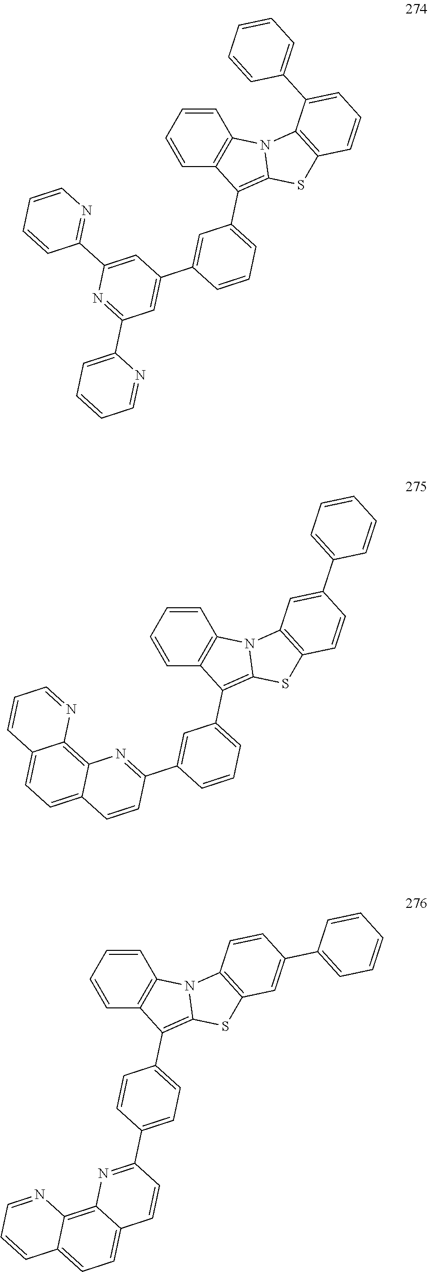 [SMILES: c1ccc(-c2cccc3sc4c(-c5cccc(-c6cc(-c7ccccn7)nc(-c7ccccn7)c6)c5)c5ccccc5n4c23)cc1 ; c1ccc(-c2ccc3sc4c(-c5cccc(-c6ccc7ccc8cccnc8c7n6)c5)c5ccccc5n4c3c2)cc1 ; c1ccc(-c2ccc3c(c2)sc2c(-c4ccc(-c5ccc6ccc7cccnc7c6n5)cc4)c4ccccc4n23)cc1]

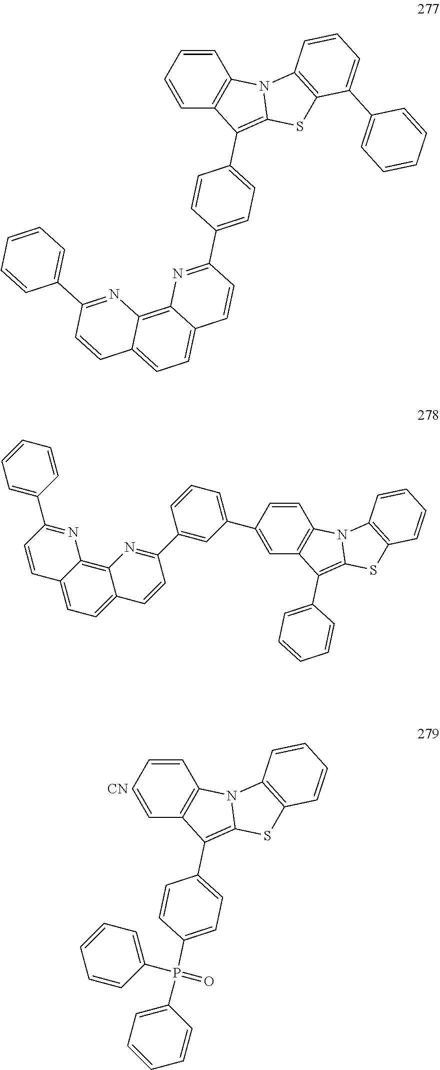

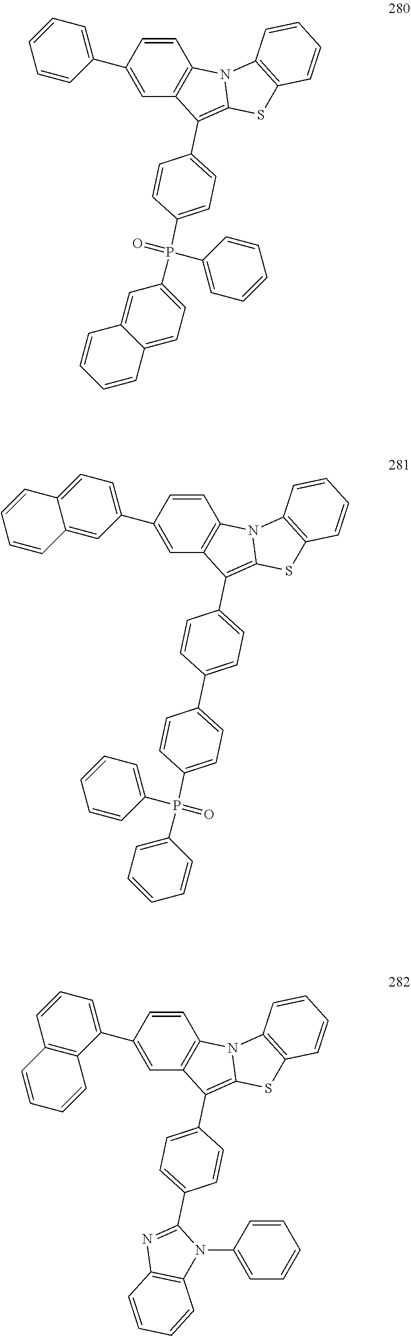 [SMILES: O=P(c1ccccc1)(c1ccc(-c2c3cc(-c4ccccc4)ccc3n3c2sc2ccccc23)cc1)c1ccc2ccccc2c1 ; O=P(c1ccccc1)(c1ccccc1)c1ccc(-c2ccc(-c3c4cc(-c5ccc6ccccc6c5)ccc4n4c3sc3ccccc34)cc2)cc1 ; c1ccc(-n2c(-c3ccc(-c4c5cc(-c6cccc7ccccc67)ccc5n5c4sc4ccccc45)cc3)nc3ccccc32)cc1]

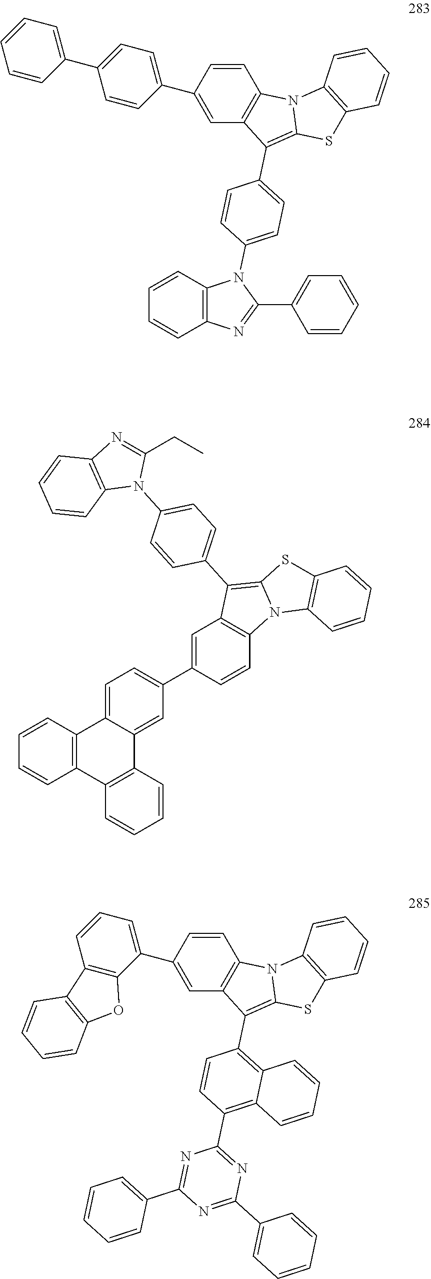 [SMILES: c1ccc(-c2ccc(-c3ccc4c(c3)c(-c3ccc(-n5c(-c6ccccc6)nc6ccccc65)cc3)c3sc5ccccc5n34)cc2)cc1 ; CCc1nc2ccccc2n1-c1ccc(-c2c3cc(-c4ccc5c6ccccc6c6ccccc6c5c4)ccc3n3c2sc2ccccc23)cc1 ; c1ccc(-c2nc(-c3ccccc3)nc(-c3ccc(-c4c5cc(-c6cccc7c6oc6ccccc67)ccc5n5c4sc4ccccc45)c4ccccc34)n2)cc1]

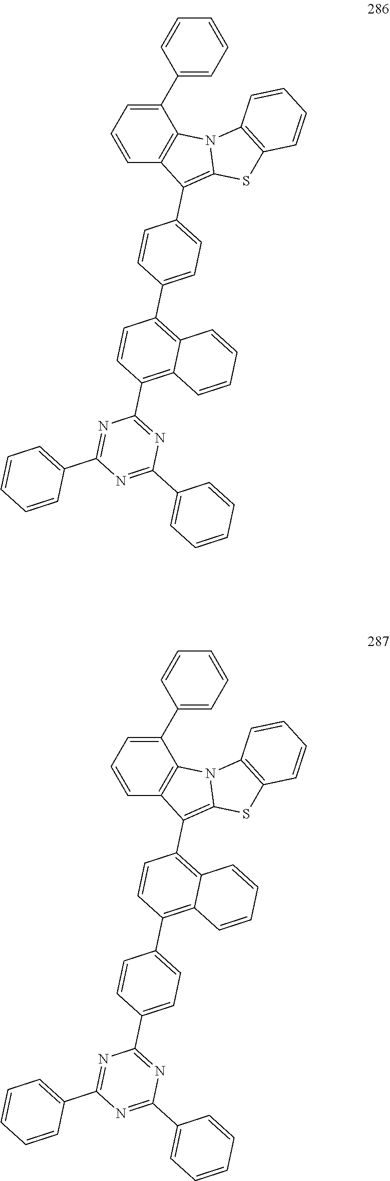 [SMILES: c1ccc(-c2nc(-c3ccccc3)nc(-c3ccc(-c4ccc(-c5c6cccc(-c7ccccc7)c6n6c5sc5ccccc56)cc4)c4ccccc34)n2)cc1 ; c1ccc(-c2nc(-c3ccccc3)nc(-c3ccc(-c4ccc(-c5c6cccc(-c7ccccc7)c6n6c5sc5ccccc56)c5ccccc45)cc3)n2)cc1]

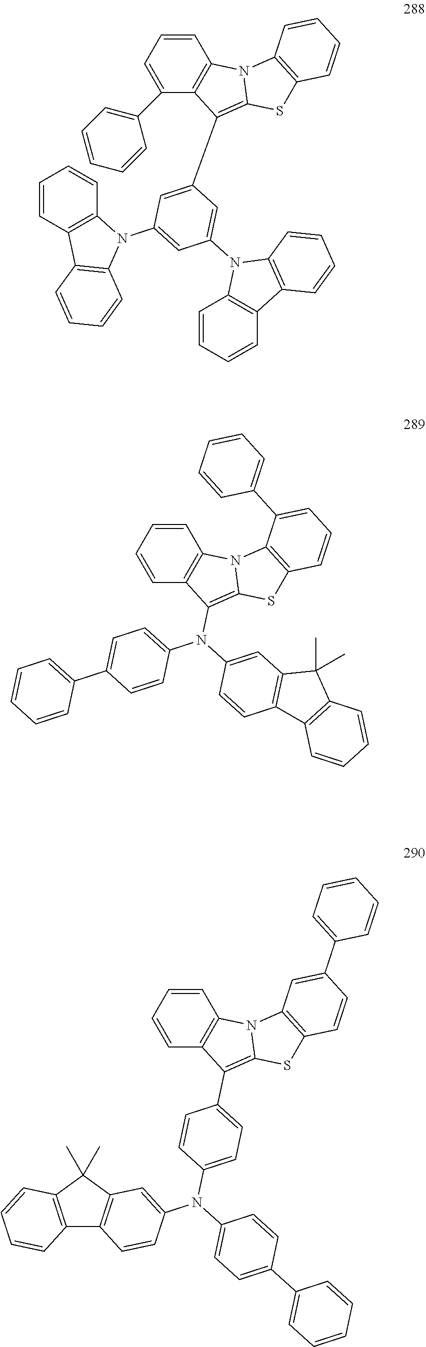 [SMILES: c1ccc(-c2cccc3c2c(-c2cc(-n4c5ccccc5c5ccccc54)cc(-n4c5ccccc5c5ccccc54)c2)c2sc4ccccc4n23)cc1 ; CC1(C)c2ccccc2-c2ccc(N(c3ccc(-c4ccccc4)cc3)c3c4ccccc4n4c3sc3cccc(-c5ccccc5)c34)cc21 ; CC1(C)c2ccccc2-c2ccc(N(c3ccc(-c4ccccc4)cc3)c3ccc(-c4c5ccccc5n5c4sc4ccc(-c6ccccc6)cc45)cc3)cc21]

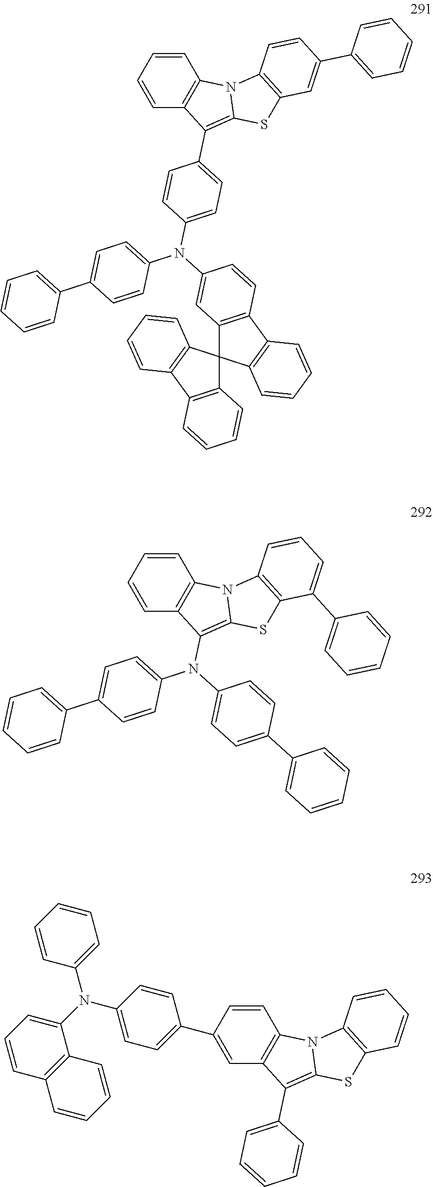 [SMILES: c1ccc(-c2ccc(N(c3ccc(-c4c5ccccc5n5c4sc4cc(-c6ccccc6)ccc45)cc3)c3ccc4c(c3)C3(c5ccccc5-c5ccccc53)c3ccccc3-4)cc2)cc1 ; c1ccc(-c2ccc(N(c3ccc(-c4ccccc4)cc3)c3c4ccccc4n4c3sc3c(-c5ccccc5)cccc34)cc2)cc1 ; c1ccc(-c2c3cc(-c4ccc(N(c5ccccc5)c5cccc6ccccc56)cc4)ccc3n3c2sc2ccccc23)cc1]

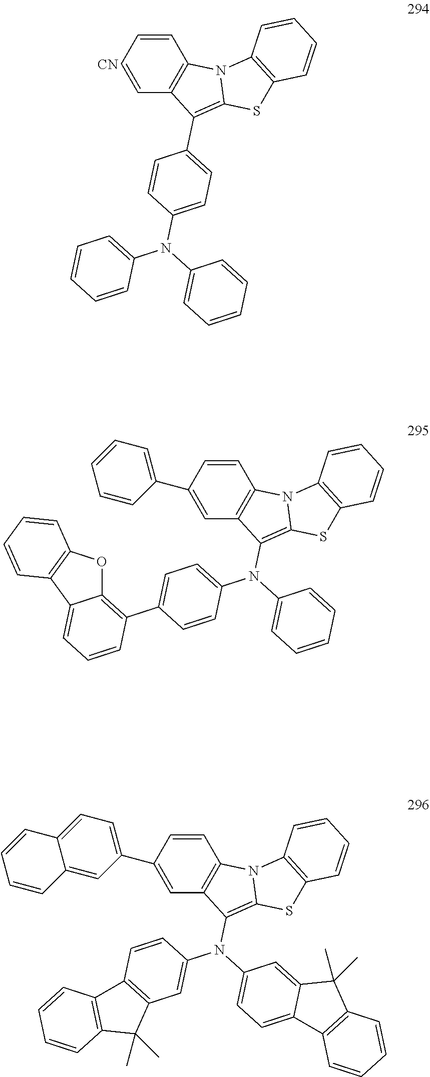

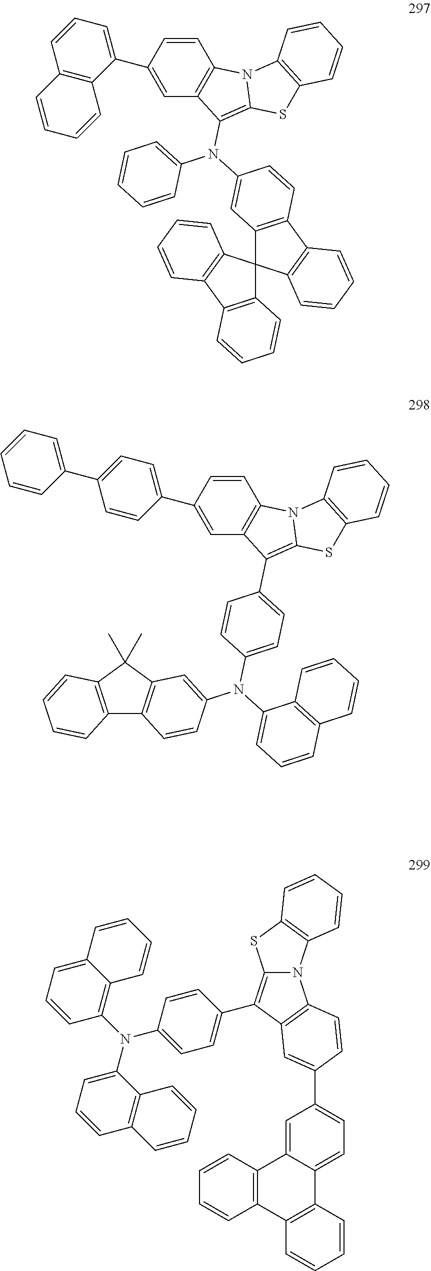 [SMILES: c1ccc(N(c2ccc3c(c2)C2(c4ccccc4-c4ccccc42)c2ccccc2-3)c2c3cc(-c4cccc5ccccc45)ccc3n3c2sc2ccccc23)cc1 ; CC1(C)c2ccccc2-c2ccc(N(c3ccc(-c4c5cc(-c6ccc(-c7ccccc7)cc6)ccc5n5c4sc4ccccc45)cc3)c3cccc4ccccc34)cc21 ; c1ccc2c(N(c3ccc(-c4c5cc(-c6ccc7c8ccccc8c8ccccc8c7c6)ccc5n5c4sc4ccccc45)cc3)c3cccc4ccccc34)cccc2c1]

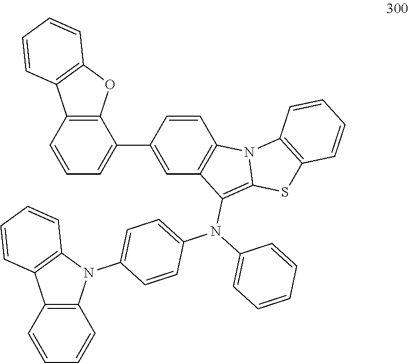 [SMILES: c1ccc(N(c2ccc(-n3c4ccccc4c4ccccc43)cc2)c2c3cc(-c4cccc5c4oc4ccccc45)ccc3n3c2sc2ccccc23)cc1]

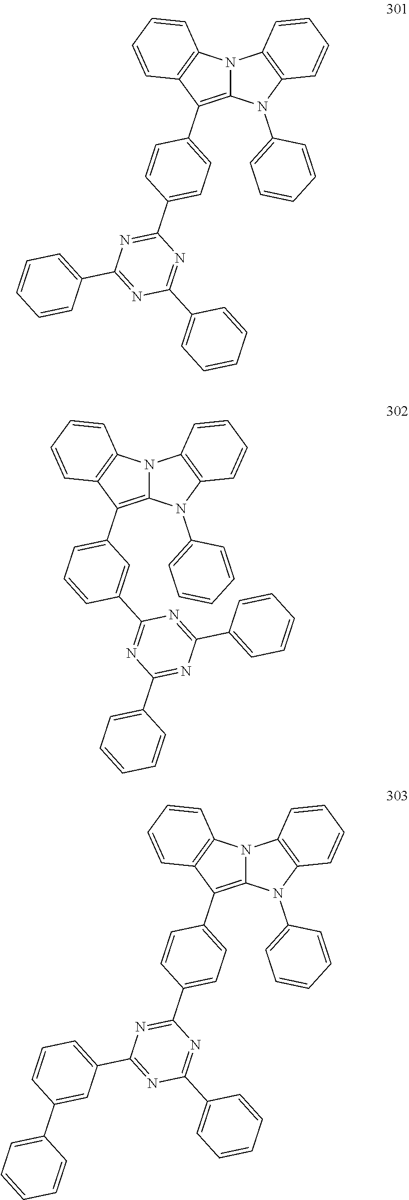

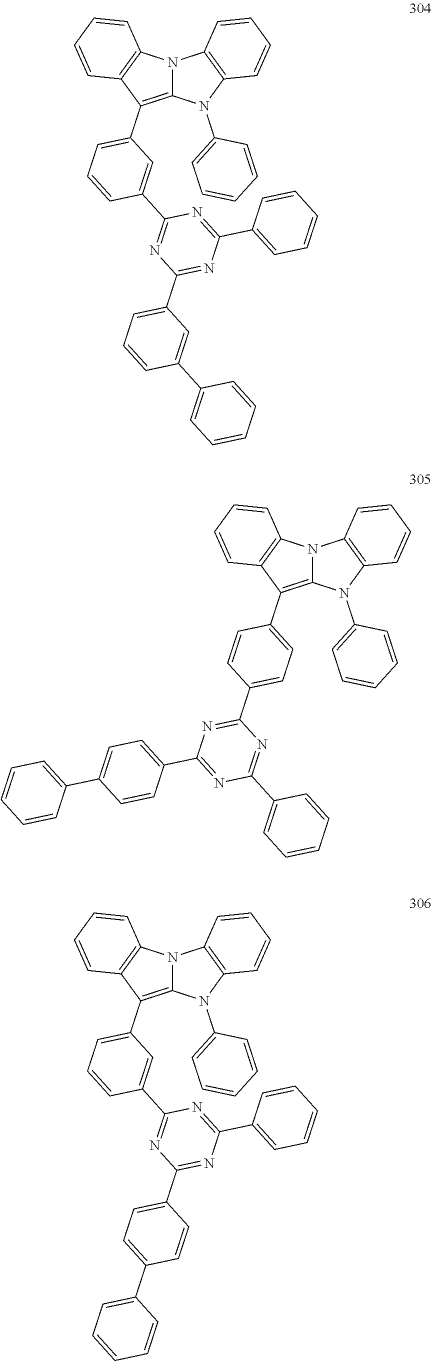 [SMILES: c1ccc(-c2cccc(-c3nc(-c4ccccc4)nc(-c4cccc(-c5c6ccccc6n6c7ccccc7n(-c7ccccc7)c56)c4)n3)c2)cc1 ; c1ccc(-c2ccc(-c3nc(-c4ccccc4)nc(-c4ccc(-c5c6ccccc6n6c7ccccc7n(-c7ccccc7)c56)cc4)n3)cc2)cc1 ; c1ccc(-c2ccc(-c3nc(-c4ccccc4)nc(-c4cccc(-c5c6ccccc6n6c7ccccc7n(-c7ccccc7)c56)c4)n3)cc2)cc1]

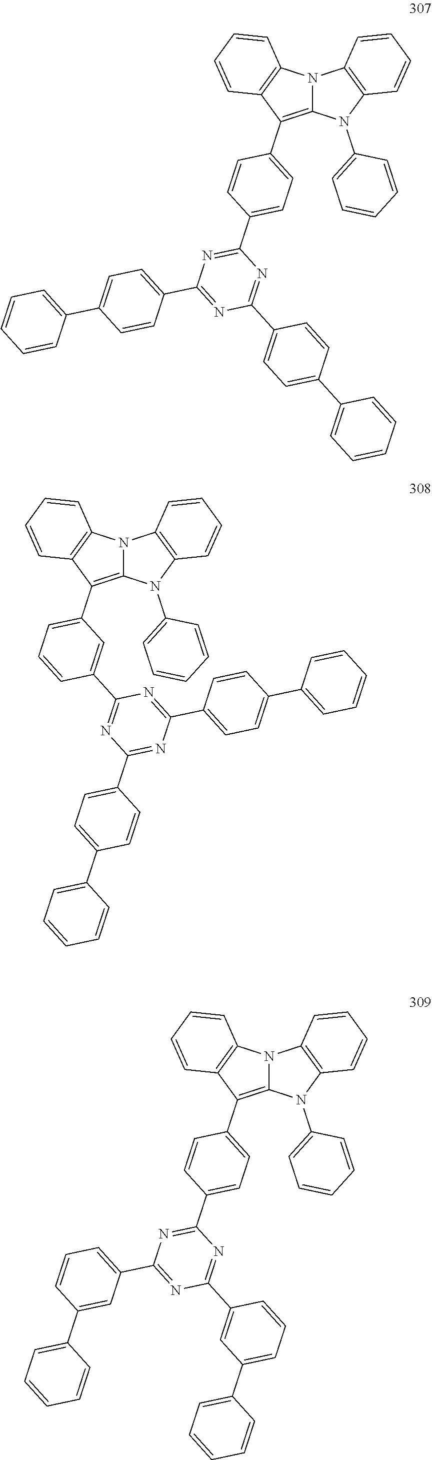 [SMILES: c1ccc(-c2ccc(-c3nc(-c4ccc(-c5ccccc5)cc4)nc(-c4ccc(-c5c6ccccc6n6c7ccccc7n(-c7ccccc7)c56)cc4)n3)cc2)cc1 ; c1ccc(-c2ccc(-c3nc(-c4ccc(-c5ccccc5)cc4)nc(-c4cccc(-c5c6ccccc6n6c7ccccc7n(-c7ccccc7)c56)c4)n3)cc2)cc1 ; c1ccc(-c2cccc(-c3nc(-c4ccc(-c5c6ccccc6n6c7ccccc7n(-c7ccccc7)c56)cc4)nc(-c4cccc(-c5ccccc5)c4)n3)c2)cc1]

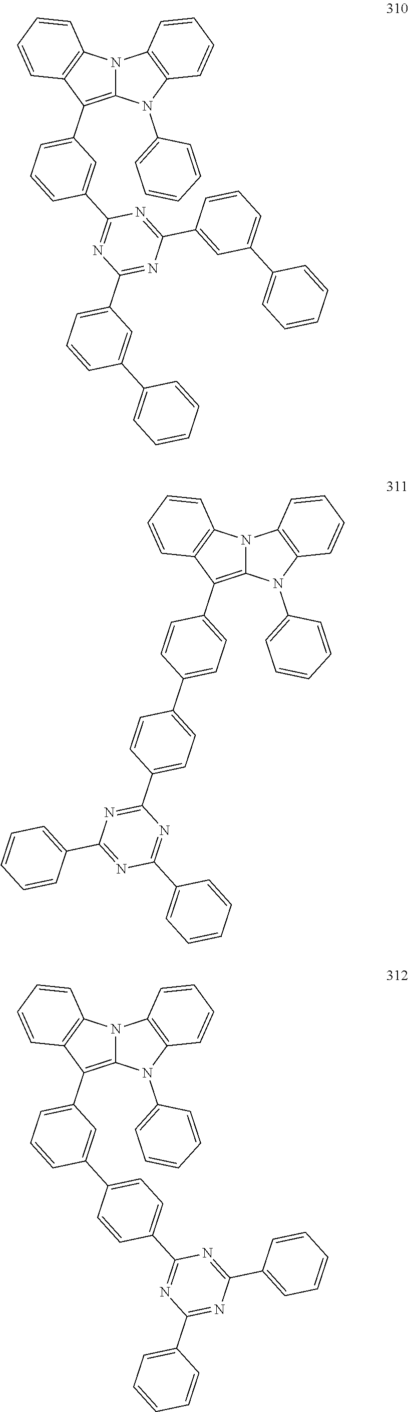

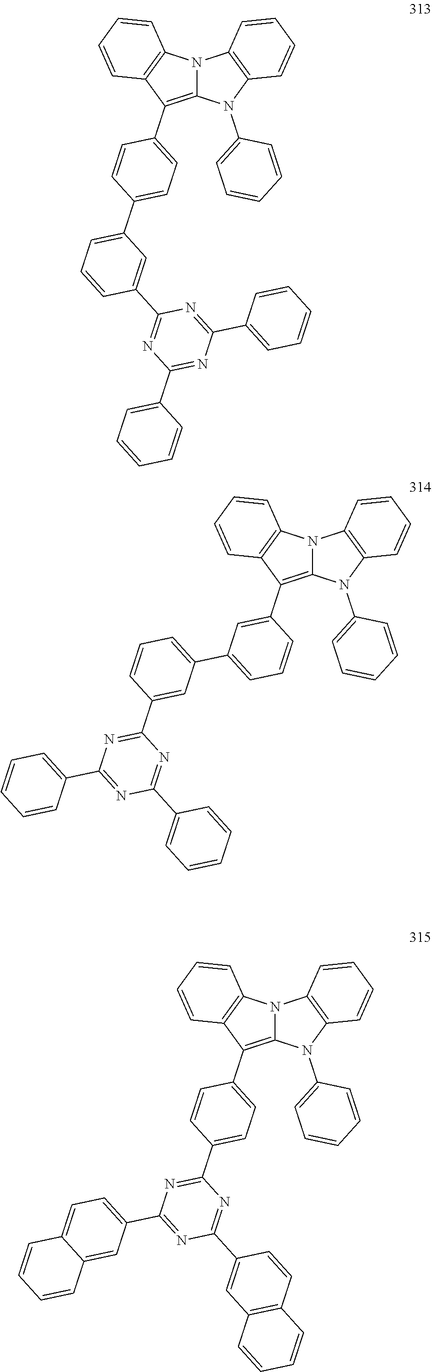 [SMILES: c1ccc(-c2nc(-c3ccccc3)nc(-c3cccc(-c4ccc(-c5c6ccccc6n6c7ccccc7n(-c7ccccc7)c56)cc4)c3)n2)cc1 ; c1ccc(-c2nc(-c3ccccc3)nc(-c3cccc(-c4cccc(-c5c6ccccc6n6c7ccccc7n(-c7ccccc7)c56)c4)c3)n2)cc1 ; c1ccc(-n2c3ccccc3n3c4ccccc4c(-c4ccc(-c5nc(-c6ccc7ccccc7c6)nc(-c6ccc7ccccc7c6)n5)cc4)c23)cc1]

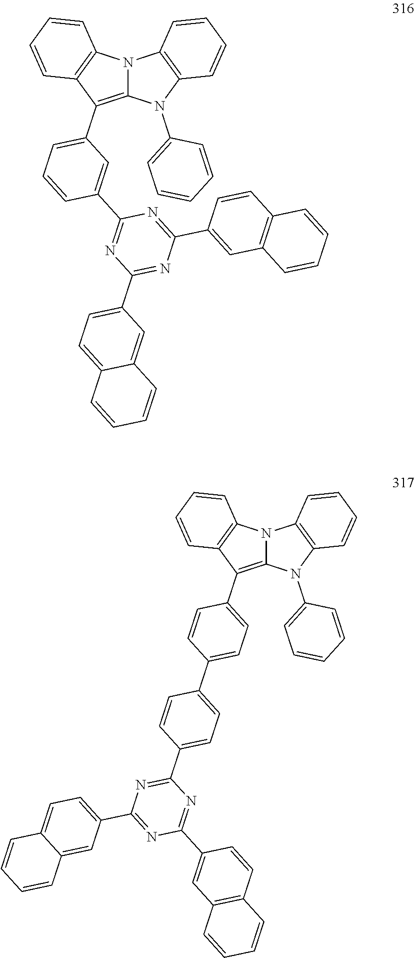 [SMILES: c1ccc(-n2c3ccccc3n3c4ccccc4c(-c4cccc(-c5nc(-c6ccc7ccccc7c6)nc(-c6ccc7ccccc7c6)n5)c4)c23)cc1 ; c1ccc(-n2c3ccccc3n3c4ccccc4c(-c4ccc(-c5ccc(-c6nc(-c7ccc8ccccc8c7)nc(-c7ccc8ccccc8c7)n6)cc5)cc4)c23)cc1]

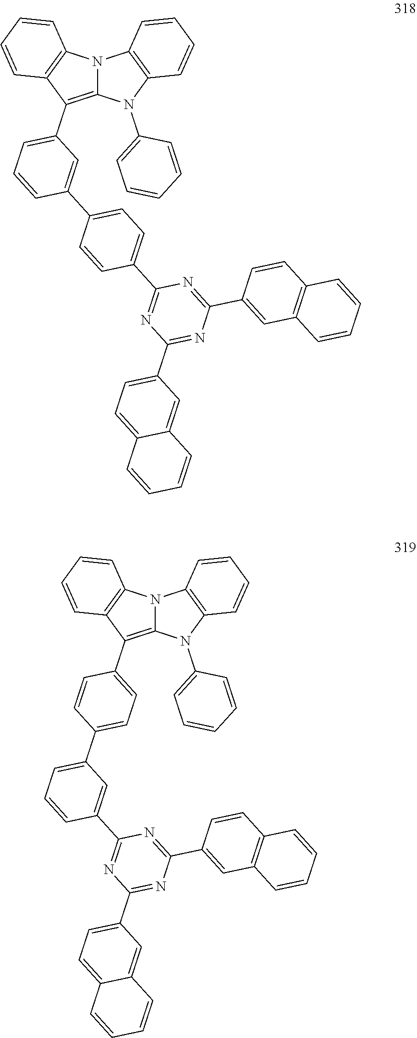 [SMILES: c1ccc(-n2c3ccccc3n3c4ccccc4c(-c4cccc(-c5ccc(-c6nc(-c7ccc8ccccc8c7)nc(-c7ccc8ccccc8c7)n6)cc5)c4)c23)cc1 ; c1ccc(-n2c3ccccc3n3c4ccccc4c(-c4ccc(-c5cccc(-c6nc(-c7ccc8ccccc8c7)nc(-c7ccc8ccccc8c7)n6)c5)cc4)c23)cc1]

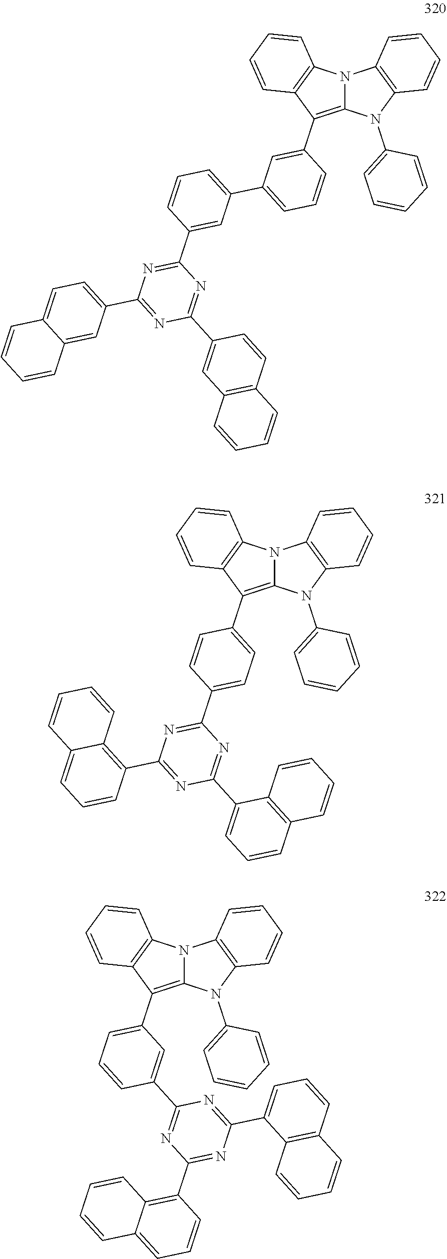 [SMILES: c1ccc(-n2c3ccccc3n3c4ccccc4c(-c4cccc(-c5cccc(-c6nc(-c7ccc8ccccc8c7)nc(-c7ccc8ccccc8c7)n6)c5)c4)c23)cc1 ; c1ccc(-n2c3ccccc3n3c4ccccc4c(-c4ccc(-c5nc(-c6cccc7ccccc67)nc(-c6cccc7ccccc67)n5)cc4)c23)cc1 ; c1ccc(-n2c3ccccc3n3c4ccccc4c(-c4cccc(-c5nc(-c6cccc7ccccc67)nc(-c6cccc7ccccc67)n5)c4)c23)cc1]

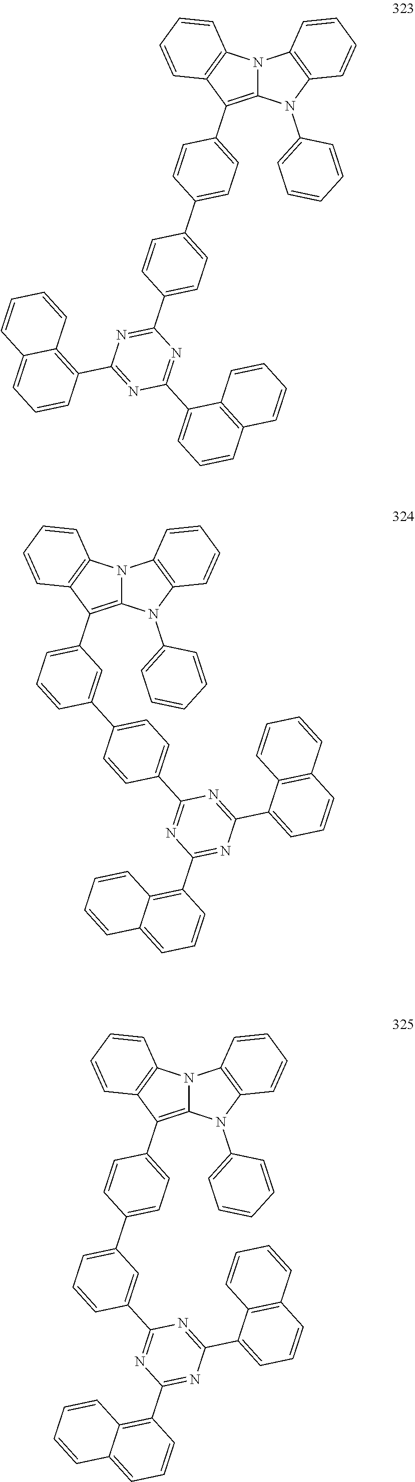 [SMILES: c1ccc(-n2c3ccccc3n3c4ccccc4c(-c4ccc(-c5ccc(-c6nc(-c7cccc8ccccc78)nc(-c7cccc8ccccc78)n6)cc5)cc4)c23)cc1 ; c1ccc(-n2c3ccccc3n3c4ccccc4c(-c4cccc(-c5ccc(-c6nc(-c7cccc8ccccc78)nc(-c7cccc8ccccc78)n6)cc5)c4)c23)cc1 ; c1ccc(-n2c3ccccc3n3c4ccccc4c(-c4ccc(-c5cccc(-c6nc(-c7cccc8ccccc78)nc(-c7cccc8ccccc78)n6)c5)cc4)c23)cc1]

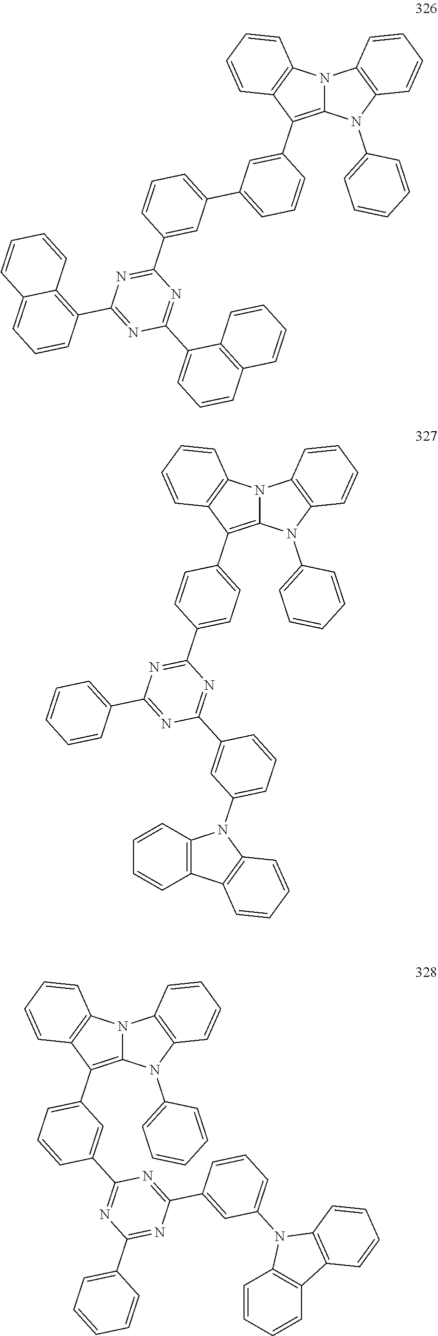

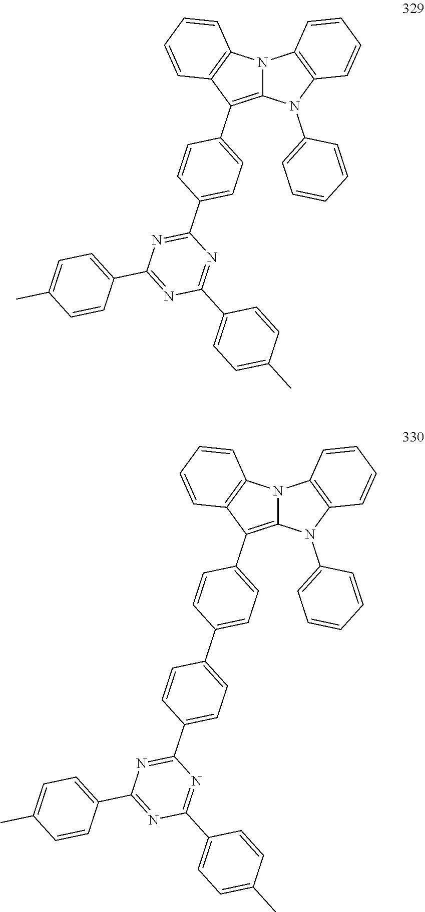 [SMILES: Cc1ccc(-c2nc(-c3ccc(C)cc3)nc(-c3ccc(-c4c5ccccc5n5c6ccccc6n(-c6ccccc6)c45)cc3)n2)cc1 ; Cc1ccc(-c2nc(-c3ccc(C)cc3)nc(-c3ccc(-c4ccc(-c5c6ccccc6n6c7ccccc7n(-c7ccccc7)c56)cc4)cc3)n2)cc1]

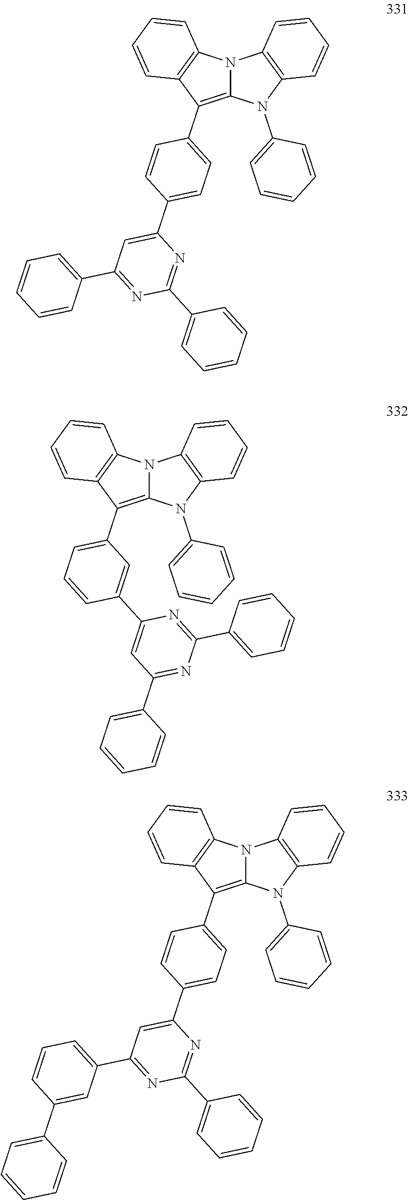

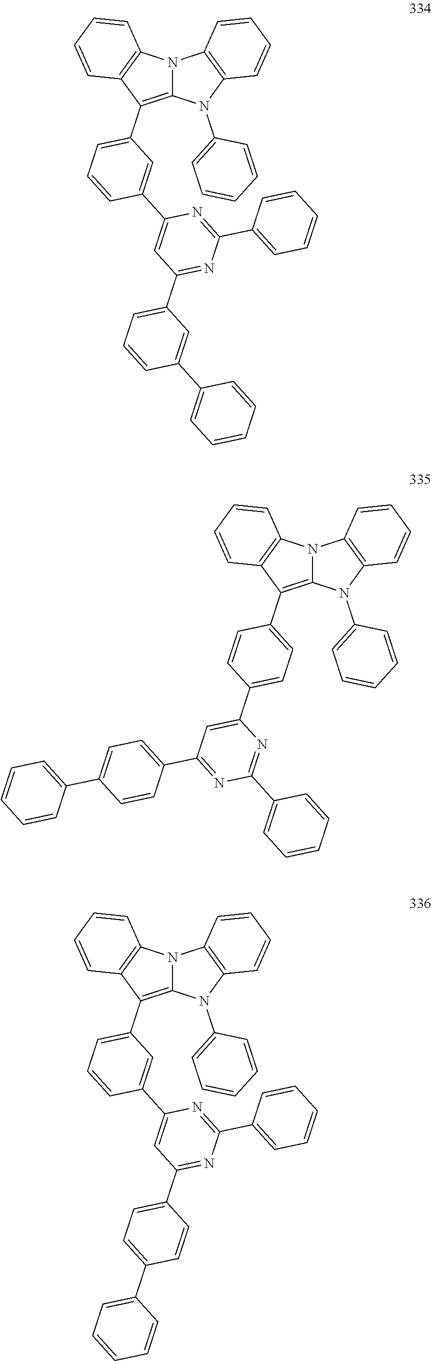 [SMILES: c1ccc(-c2cccc(-c3cc(-c4cccc(-c5c6ccccc6n6c7ccccc7n(-c7ccccc7)c56)c4)nc(-c4ccccc4)n3)c2)cc1 ; c1ccc(-c2ccc(-c3cc(-c4ccc(-c5c6ccccc6n6c7ccccc7n(-c7ccccc7)c56)cc4)nc(-c4ccccc4)n3)cc2)cc1 ; c1ccc(-c2ccc(-c3cc(-c4cccc(-c5c6ccccc6n6c7ccccc7n(-c7ccccc7)c56)c4)nc(-c4ccccc4)n3)cc2)cc1]

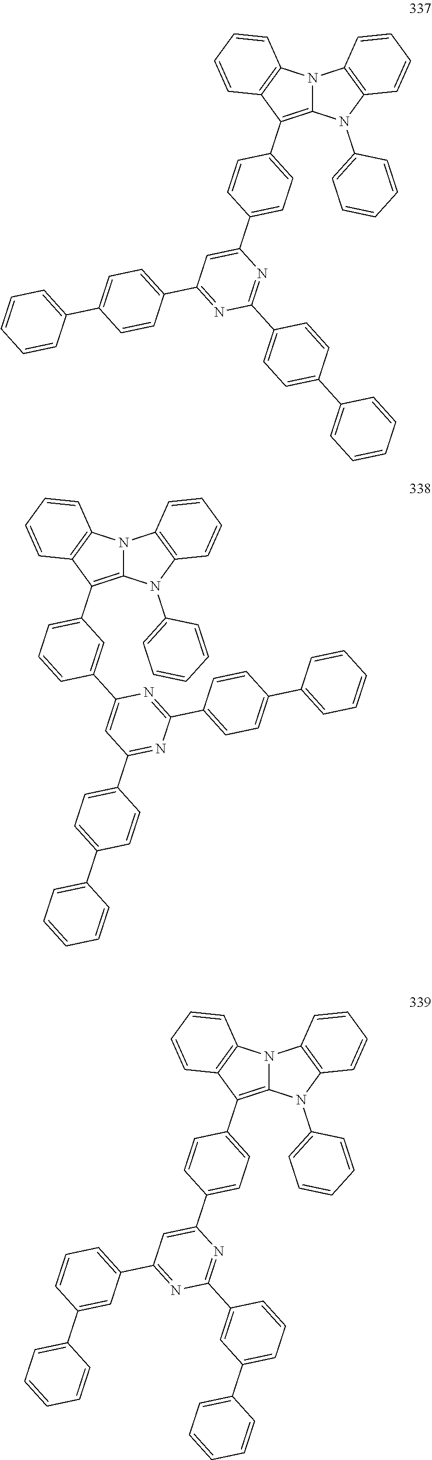 [SMILES: c1ccc(-c2ccc(-c3cc(-c4ccc(-c5c6ccccc6n6c7ccccc7n(-c7ccccc7)c56)cc4)nc(-c4ccc(-c5ccccc5)cc4)n3)cc2)cc1 ; c1ccc(-c2ccc(-c3cc(-c4cccc(-c5c6ccccc6n6c7ccccc7n(-c7ccccc7)c56)c4)nc(-c4ccc(-c5ccccc5)cc4)n3)cc2)cc1 ; c1ccc(-c2cccc(-c3cc(-c4ccc(-c5c6ccccc6n6c7ccccc7n(-c7ccccc7)c56)cc4)nc(-c4cccc(-c5ccccc5)c4)n3)c2)cc1]

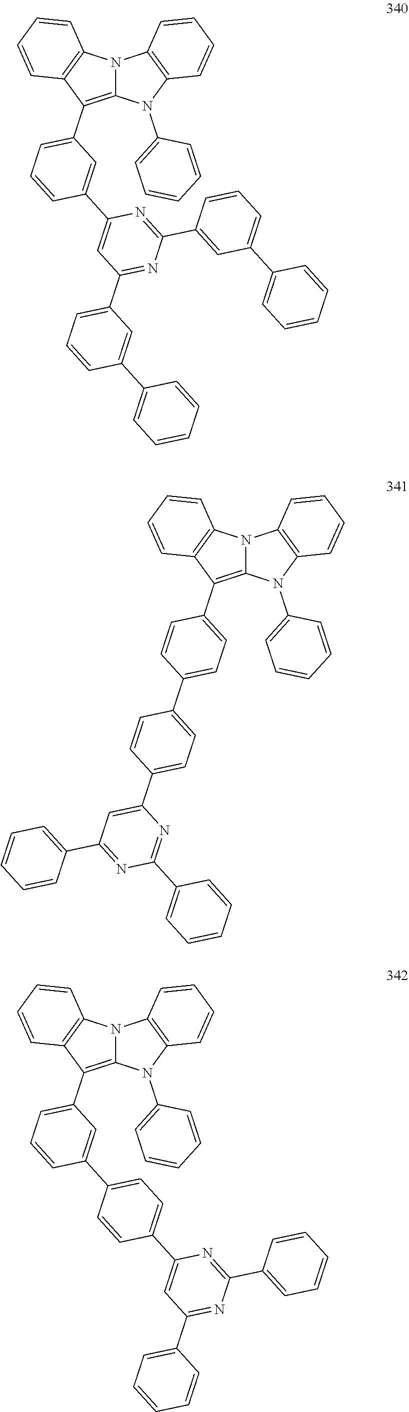

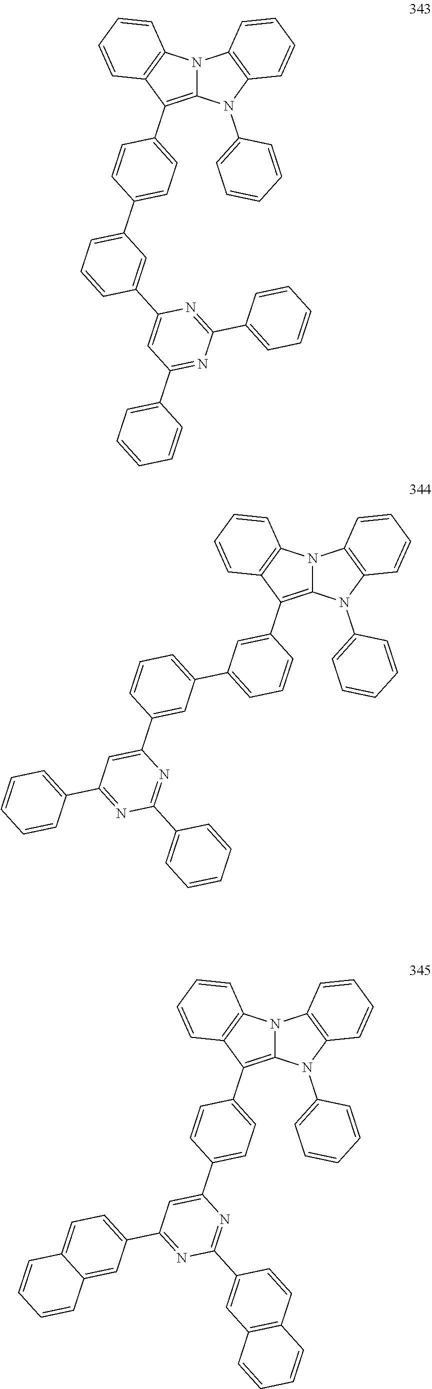 [SMILES: c1ccc(-c2cc(-c3cccc(-c4ccc(-c5c6ccccc6n6c7ccccc7n(-c7ccccc7)c56)cc4)c3)nc(-c3ccccc3)n2)cc1 ; c1ccc(-c2cc(-c3cccc(-c4cccc(-c5c6ccccc6n6c7ccccc7n(-c7ccccc7)c56)c4)c3)nc(-c3ccccc3)n2)cc1 ; c1ccc(-n2c3ccccc3n3c4ccccc4c(-c4ccc(-c5cc(-c6ccc7ccccc7c6)nc(-c6ccc7ccccc7c6)n5)cc4)c23)cc1]

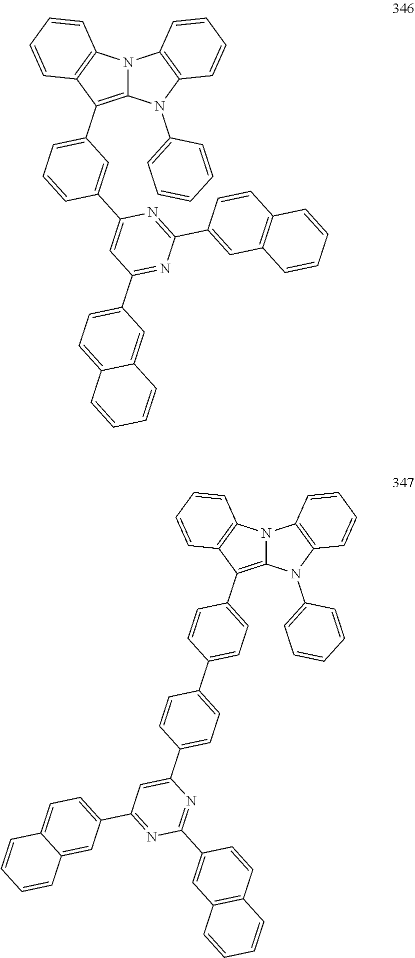 [SMILES: c1ccc(-n2c3ccccc3n3c4ccccc4c(-c4cccc(-c5cc(-c6ccc7ccccc7c6)nc(-c6ccc7ccccc7c6)n5)c4)c23)cc1 ; c1ccc(-n2c3ccccc3n3c4ccccc4c(-c4ccc(-c5ccc(-c6cc(-c7ccc8ccccc8c7)nc(-c7ccc8ccccc8c7)n6)cc5)cc4)c23)cc1]

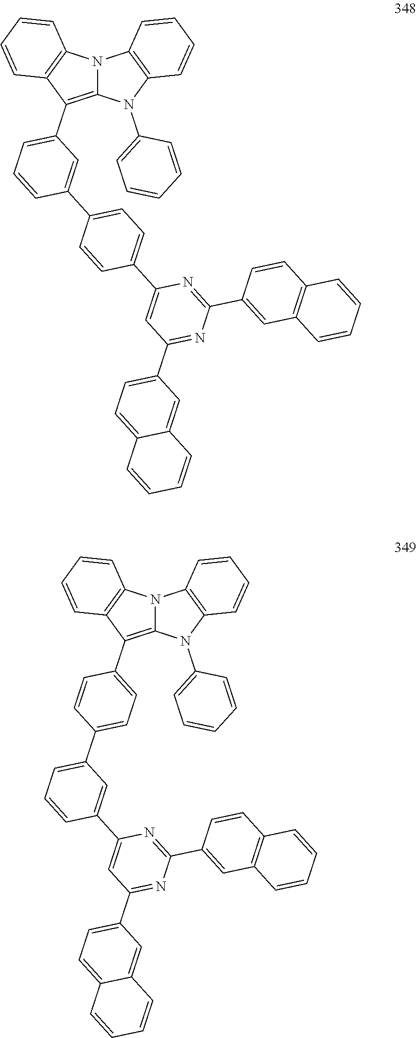 [SMILES: c1ccc(-n2c3ccccc3n3c4ccccc4c(-c4cccc(-c5ccc(-c6cc(-c7ccc8ccccc8c7)nc(-c7ccc8ccccc8c7)n6)cc5)c4)c23)cc1 ; c1ccc(-n2c3ccccc3n3c4ccccc4c(-c4ccc(-c5cccc(-c6cc(-c7ccc8ccccc8c7)nc(-c7ccc8ccccc8c7)n6)c5)cc4)c23)cc1]

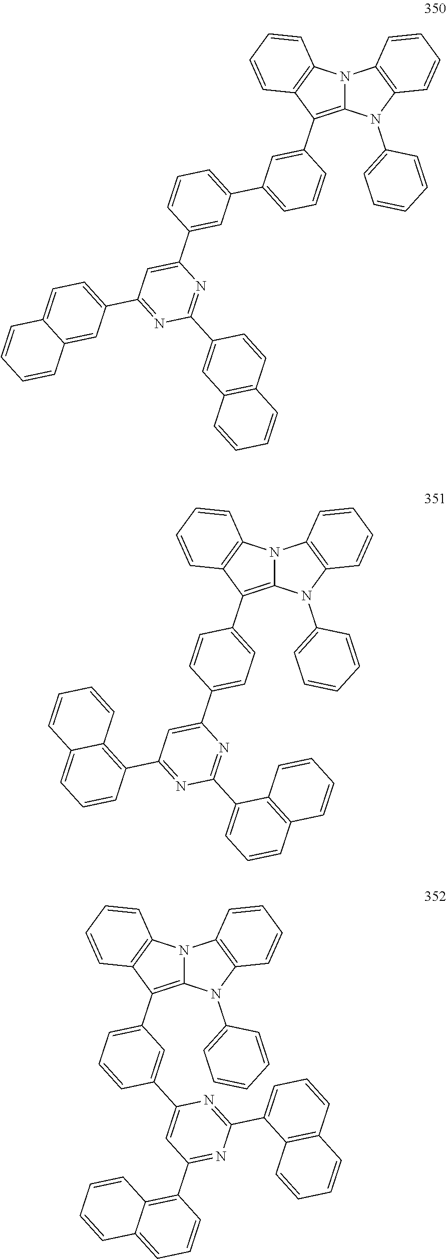 [SMILES: c1ccc(-n2c3ccccc3n3c4ccccc4c(-c4cccc(-c5cccc(-c6cc(-c7ccc8ccccc8c7)nc(-c7ccc8ccccc8c7)n6)c5)c4)c23)cc1 ; c1ccc(-n2c3ccccc3n3c4ccccc4c(-c4ccc(-c5cc(-c6cccc7ccccc67)nc(-c6cccc7ccccc67)n5)cc4)c23)cc1 ; c1ccc(-n2c3ccccc3n3c4ccccc4c(-c4cccc(-c5cc(-c6cccc7ccccc67)nc(-c6cccc7ccccc67)n5)c4)c23)cc1]

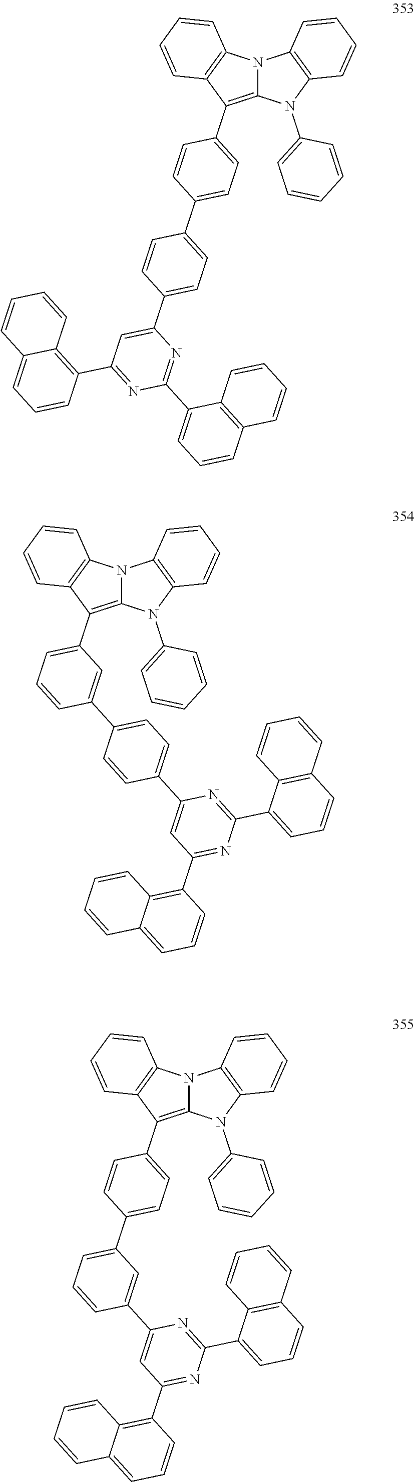 [SMILES: c1ccc(-n2c3ccccc3n3c4ccccc4c(-c4ccc(-c5ccc(-c6cc(-c7cccc8ccccc78)nc(-c7cccc8ccccc78)n6)cc5)cc4)c23)cc1 ; c1ccc(-n2c3ccccc3n3c4ccccc4c(-c4cccc(-c5ccc(-c6cc(-c7cccc8ccccc78)nc(-c7cccc8ccccc78)n6)cc5)c4)c23)cc1 ; c1ccc(-n2c3ccccc3n3c4ccccc4c(-c4ccc(-c5cccc(-c6cc(-c7cccc8ccccc78)nc(-c7cccc8ccccc78)n6)c5)cc4)c23)cc1]

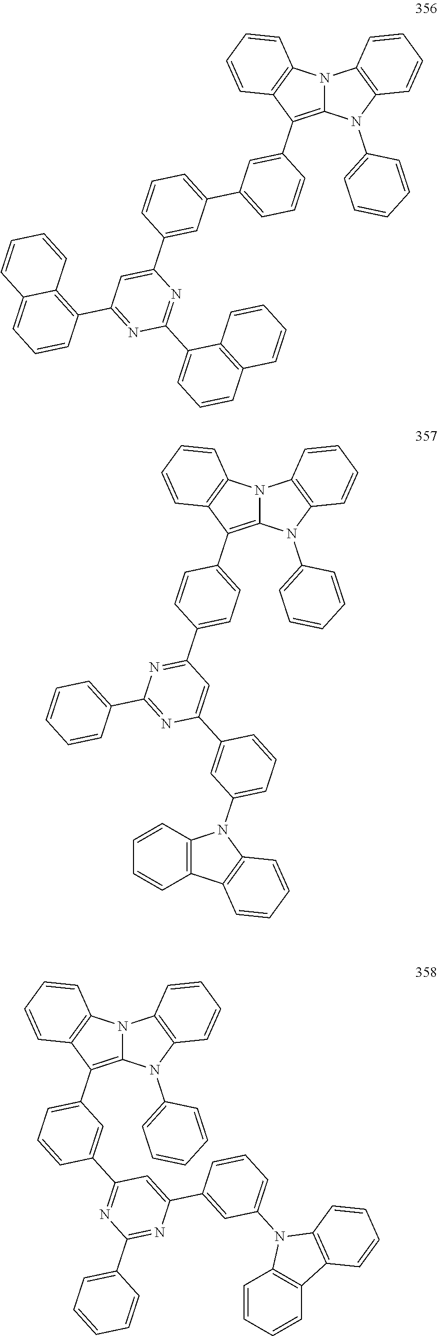

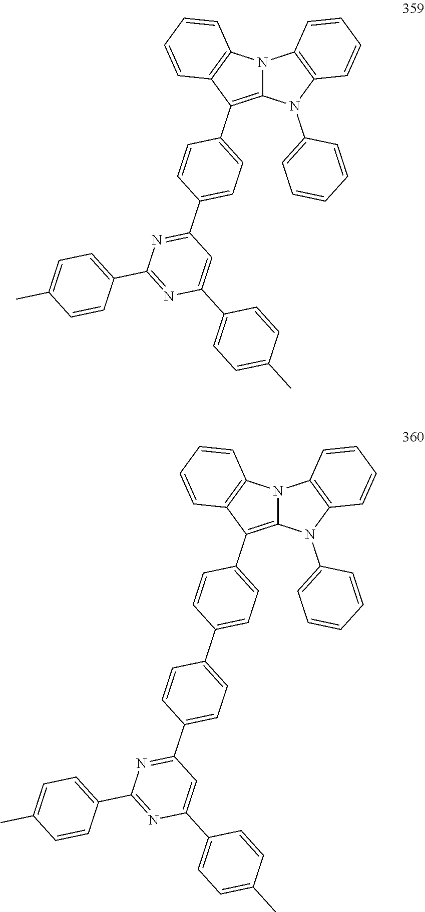 [SMILES: Cc1ccc(-c2cc(-c3ccc(-c4c5ccccc5n5c6ccccc6n(-c6ccccc6)c45)cc3)nc(-c3ccc(C)cc3)n2)cc1 ; Cc1ccc(-c2cc(-c3ccc(-c4ccc(-c5c6ccccc6n6c7ccccc7n(-c7ccccc7)c56)cc4)cc3)nc(-c3ccc(C)cc3)n2)cc1]

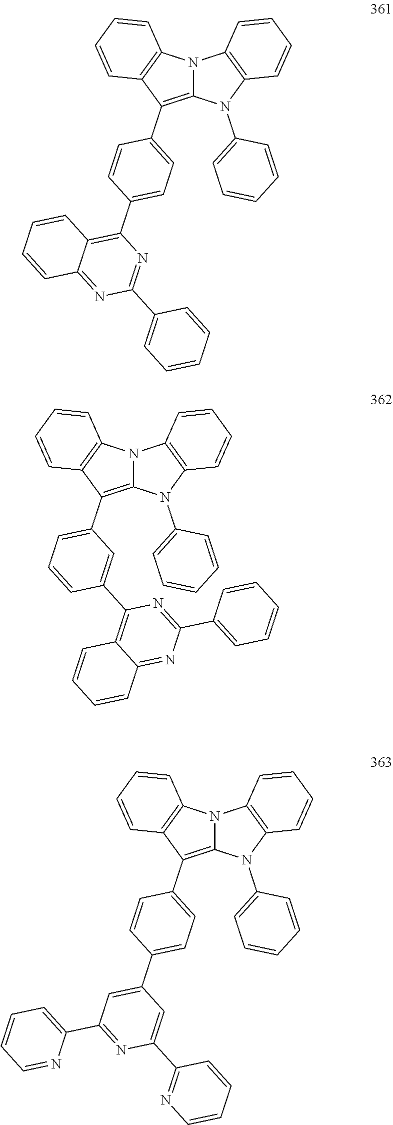

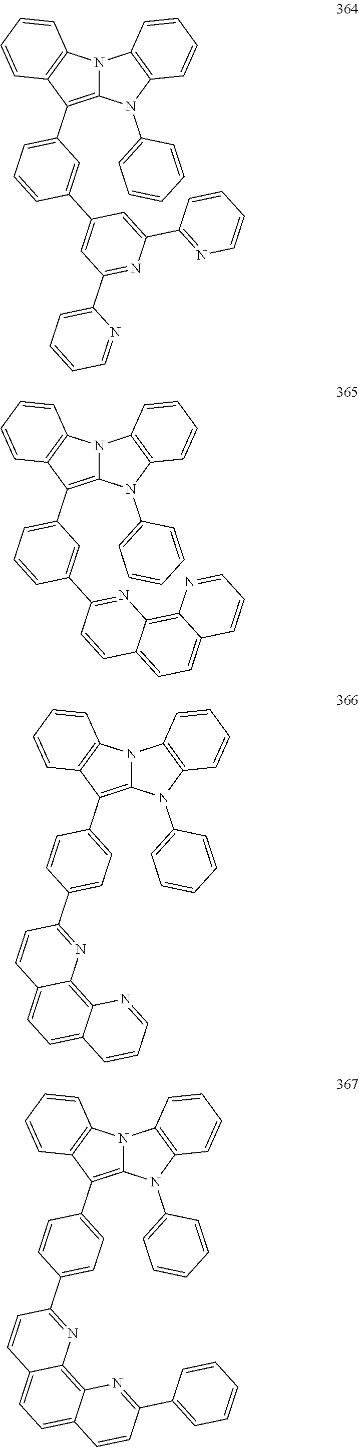

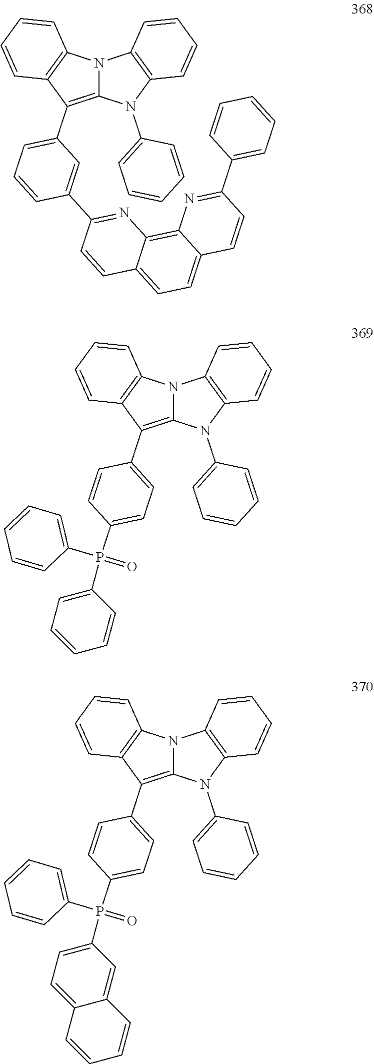 [SMILES: c1ccc(-c2ccc3ccc4ccc(-c5cccc(-c6c7ccccc7n7c8ccccc8n(-c8ccccc8)c67)c5)nc4c3n2)cc1 ; O=P(c1ccccc1)(c1ccccc1)c1ccc(-c2c3ccccc3n3c4ccccc4n(-c4ccccc4)c23)cc1 ; O=P(c1ccccc1)(c1ccc(-c2c3ccccc3n3c4ccccc4n(-c4ccccc4)c23)cc1)c1ccc2ccccc2c1]

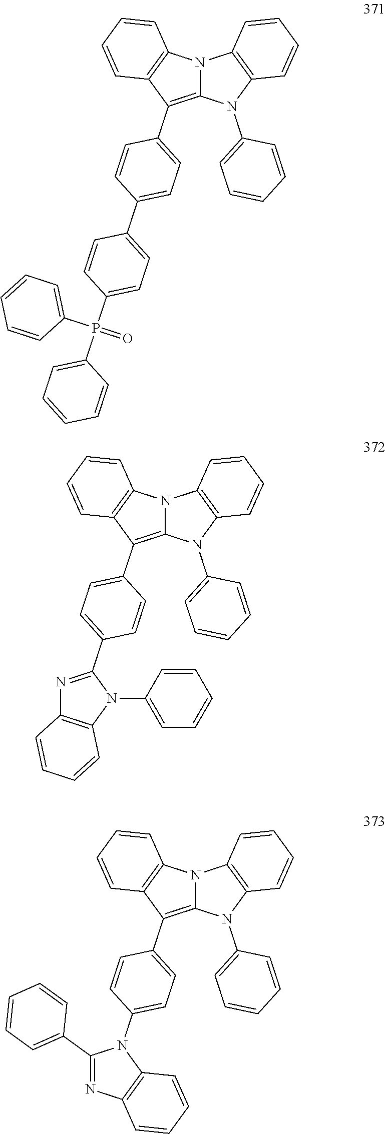

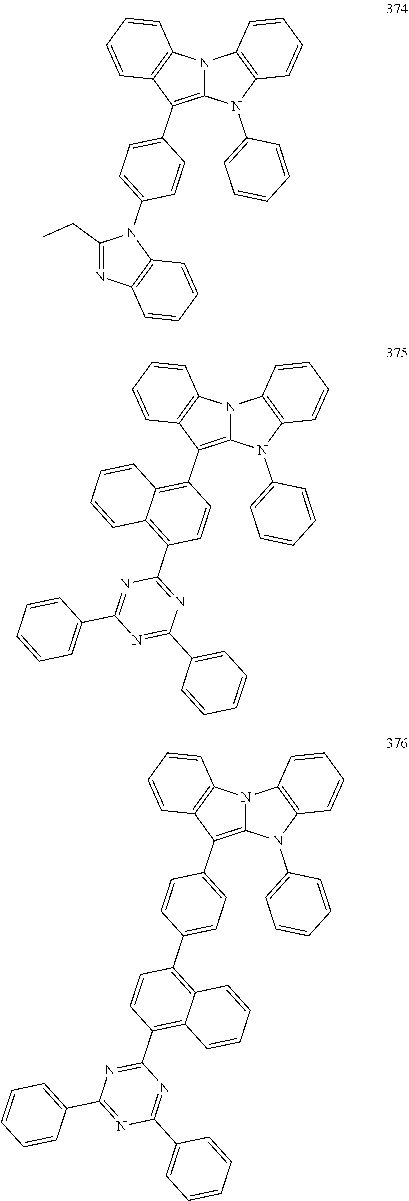

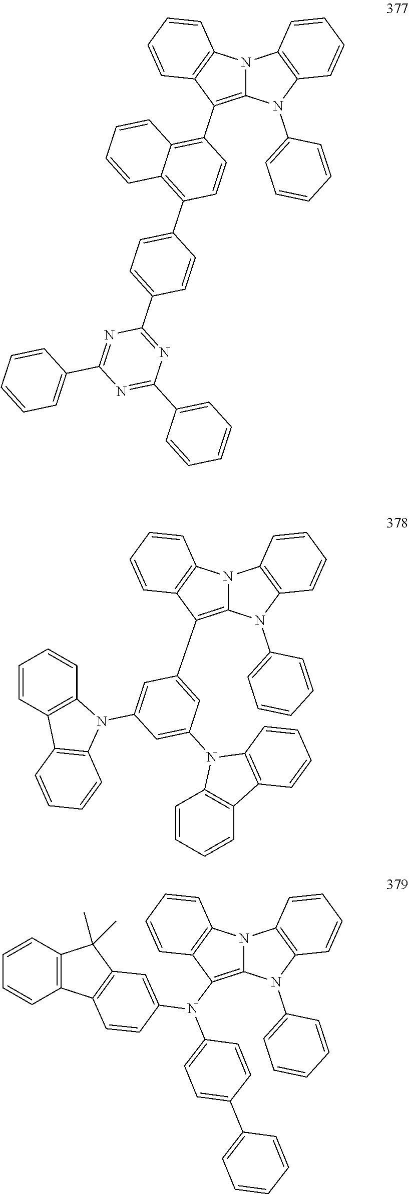

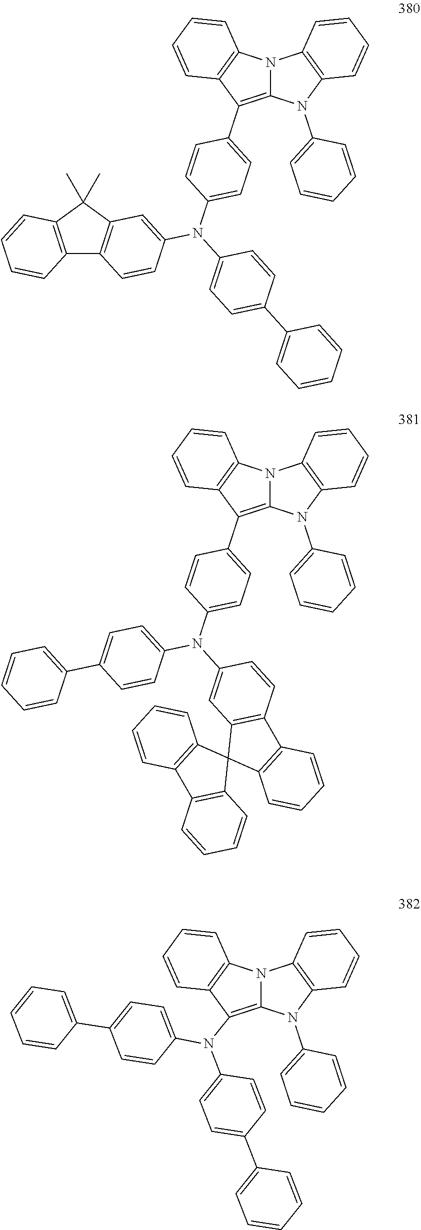 [SMILES: CC1(C)c2ccccc2-c2ccc(N(c3ccc(-c4ccccc4)cc3)c3ccc(-c4c5ccccc5n5c6ccccc6n(-c6ccccc6)c45)cc3)cc21 ; c1ccc(-c2ccc(N(c3ccc(-c4c5ccccc5n5c6ccccc6n(-c6ccccc6)c45)cc3)c3ccc4c(c3)C3(c5ccccc5-c5ccccc53)c3ccccc3-4)cc2)cc1 ; c1ccc(-c2ccc(N(c3ccc(-c4ccccc4)cc3)c3c4ccccc4n4c5ccccc5n(-c5ccccc5)c34)cc2)cc1]

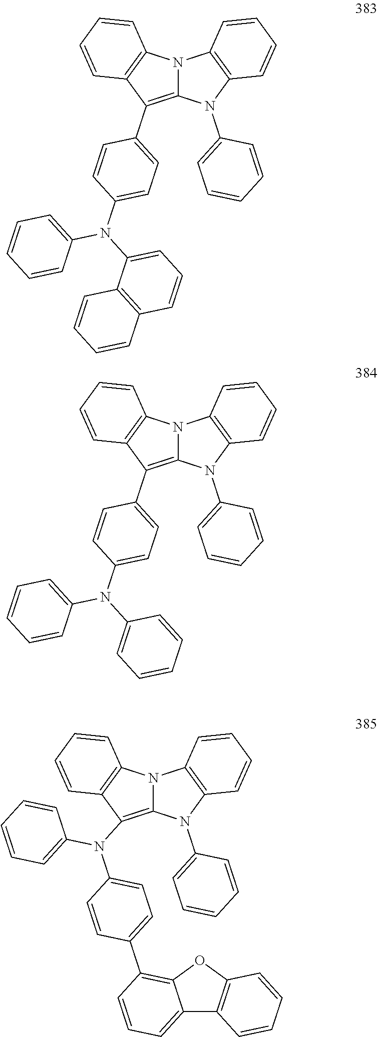

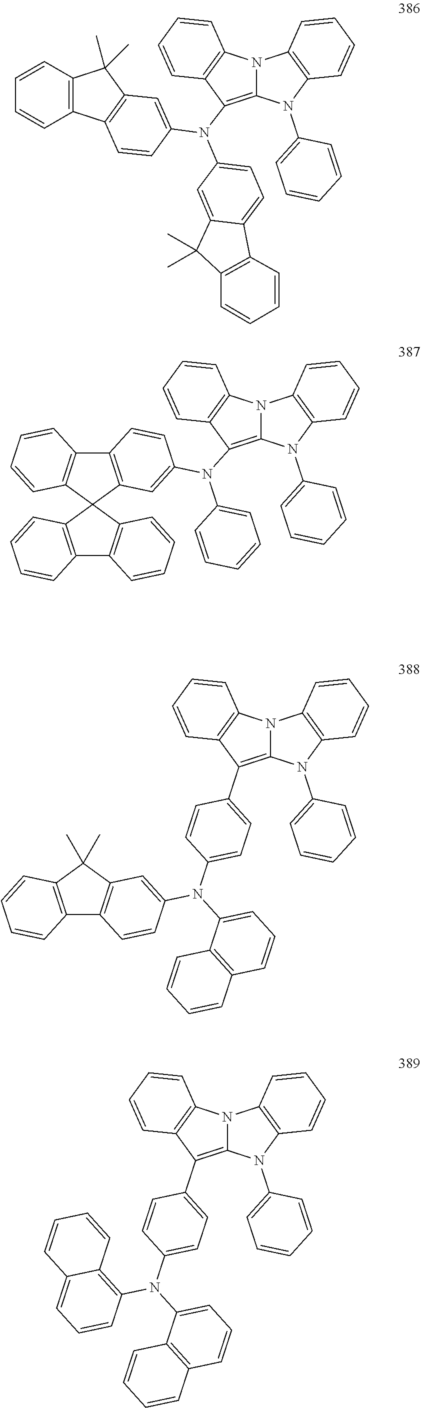 [SMILES: CC1(C)c2ccccc2-c2ccc(N(c3ccc4c(c3)C(C)(C)c3ccccc3-4)c3c4ccccc4n4c5ccccc5n(-c5ccccc5)c34)cc21 ; c1ccc(N(c2ccc3c(c2)C2(c4ccccc4-c4ccccc42)c2ccccc2-3)c2c3ccccc3n3c4ccccc4n(-c4ccccc4)c23)cc1 ; CC1(C)c2ccccc2-c2ccc(N(c3ccc(-c4c5ccccc5n5c6ccccc6n(-c6ccccc6)c45)cc3)c3cccc4ccccc34)cc21 ; c1ccc(-n2c3ccccc3n3c4ccccc4c(-c4ccc(N(c5cccc6ccccc56)c5cccc6ccccc56)cc4)c23)cc1]

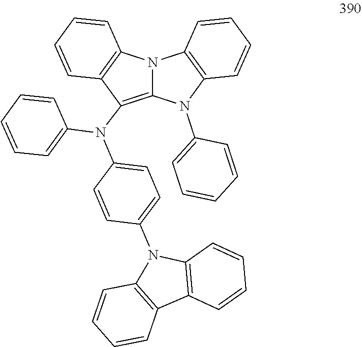 [SMILES: c1ccc(N(c2ccc(-n3c4ccccc4c4ccccc43)cc2)c2c3ccccc3n3c4ccccc4n(-c4ccccc4)c23)cc1]

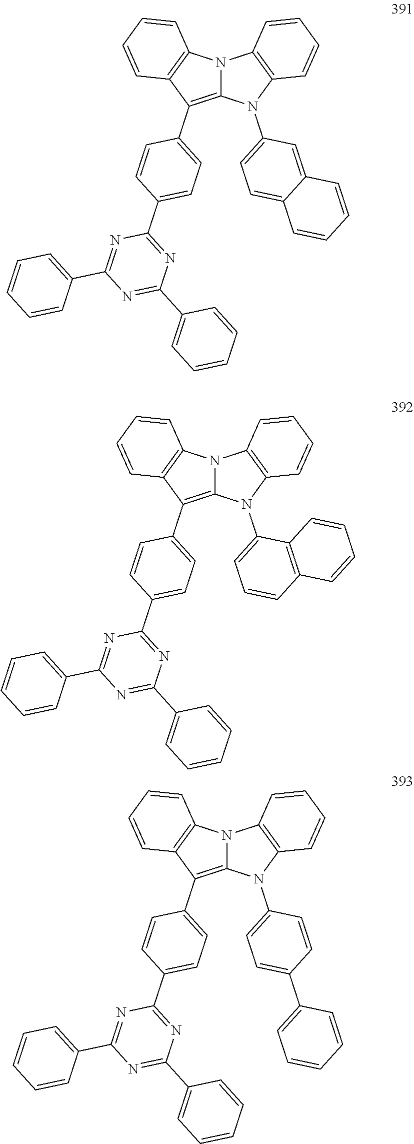

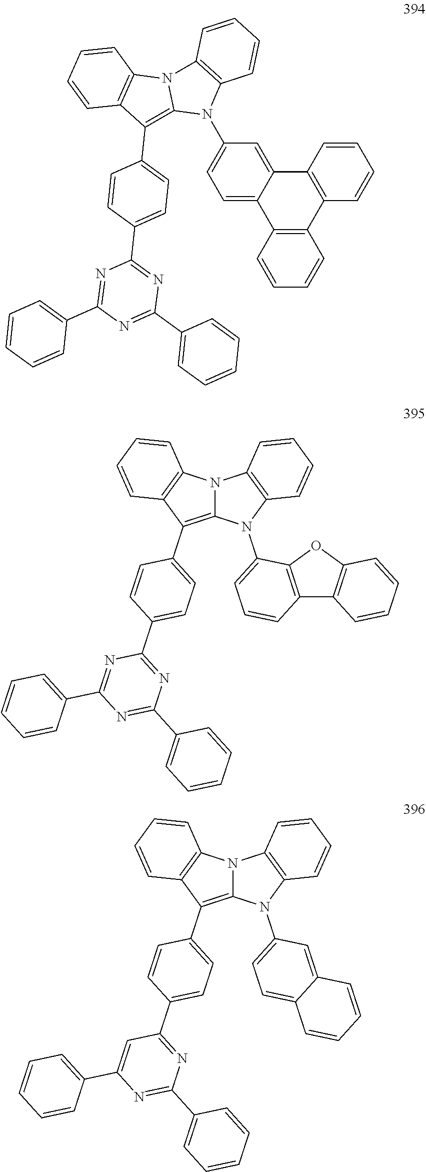

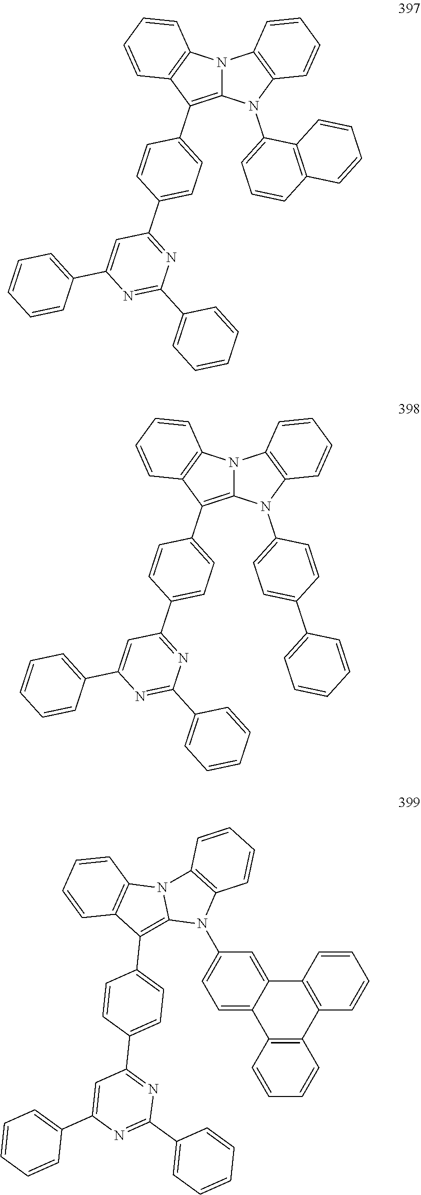 [SMILES: c1ccc(-c2cc(-c3ccc(-c4c5ccccc5n5c6ccccc6n(-c6cccc7ccccc67)c45)cc3)nc(-c3ccccc3)n2)cc1 ; c1ccc(-c2ccc(-n3c4ccccc4n4c5ccccc5c(-c5ccc(-c6cc(-c7ccccc7)nc(-c7ccccc7)n6)cc5)c34)cc2)cc1 ; c1ccc(-c2cc(-c3ccc(-c4c5ccccc5n5c6ccccc6n(-c6ccc7c8ccccc8c8ccccc8c7c6)c45)cc3)nc(-c3ccccc3)n2)cc1]

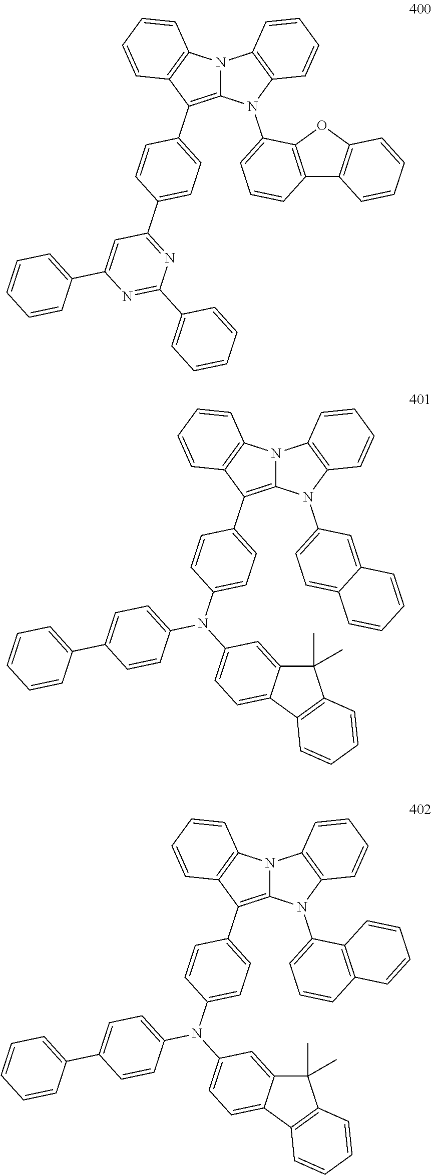

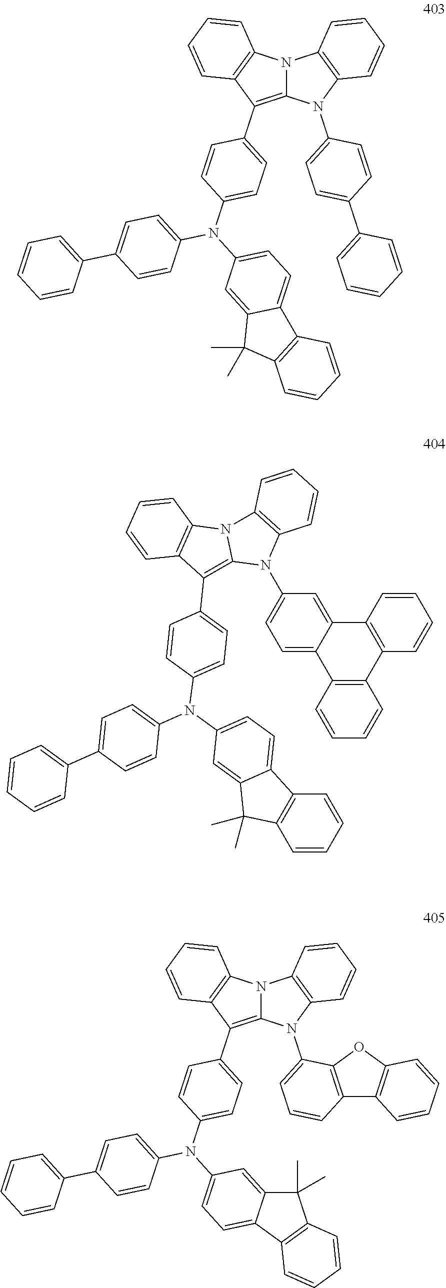 [SMILES: CC1(C)c2ccccc2-c2ccc(N(c3ccc(-c4ccccc4)cc3)c3ccc(-c4c5ccccc5n5c6ccccc6n(-c6ccc(-c7ccccc7)cc6)c45)cc3)cc21 ; CC1(C)c2ccccc2-c2ccc(N(c3ccc(-c4ccccc4)cc3)c3ccc(-c4c5ccccc5n5c6ccccc6n(-c6ccc7c8ccccc8c8ccccc8c7c6)c45)cc3)cc21 ; CC1(C)c2ccccc2-c2ccc(N(c3ccc(-c4ccccc4)cc3)c3ccc(-c4c5ccccc5n5c6ccccc6n(-c6cccc7c6oc6ccccc67)c45)cc3)cc21]

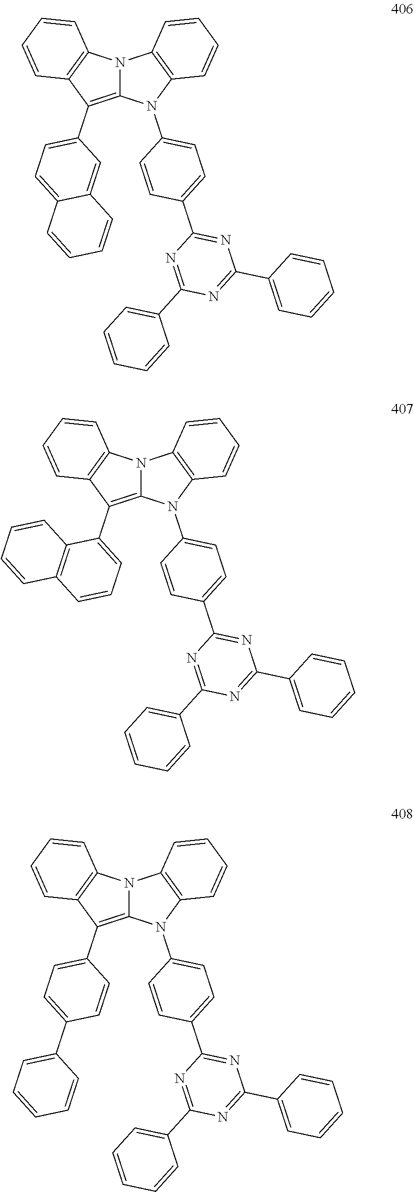 [SMILES: c1ccc(-c2nc(-c3ccccc3)nc(-c3ccc(-n4c5ccccc5n5c6ccccc6c(-c6ccc7ccccc7c6)c45)cc3)n2)cc1 ; c1ccc(-c2nc(-c3ccccc3)nc(-c3ccc(-n4c5ccccc5n5c6ccccc6c(-c6cccc7ccccc67)c45)cc3)n2)cc1 ; c1ccc(-c2ccc(-c3c4ccccc4n4c5ccccc5n(-c5ccc(-c6nc(-c7ccccc7)nc(-c7ccccc7)n6)cc5)c34)cc2)cc1]

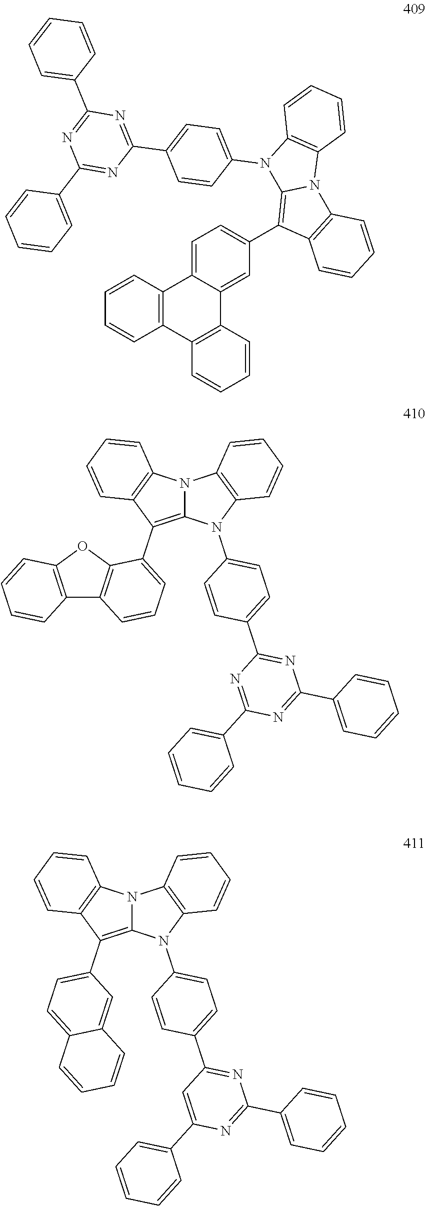 [SMILES: c1ccc(-c2nc(-c3ccccc3)nc(-c3ccc(-n4c5ccccc5n5c6ccccc6c(-c6ccc7c8ccccc8c8ccccc8c7c6)c45)cc3)n2)cc1 ; c1ccc(-c2nc(-c3ccccc3)nc(-c3ccc(-n4c5ccccc5n5c6ccccc6c(-c6cccc7c6oc6ccccc67)c45)cc3)n2)cc1 ; c1ccc(-c2cc(-c3ccc(-n4c5ccccc5n5c6ccccc6c(-c6ccc7ccccc7c6)c45)cc3)nc(-c3ccccc3)n2)cc1]

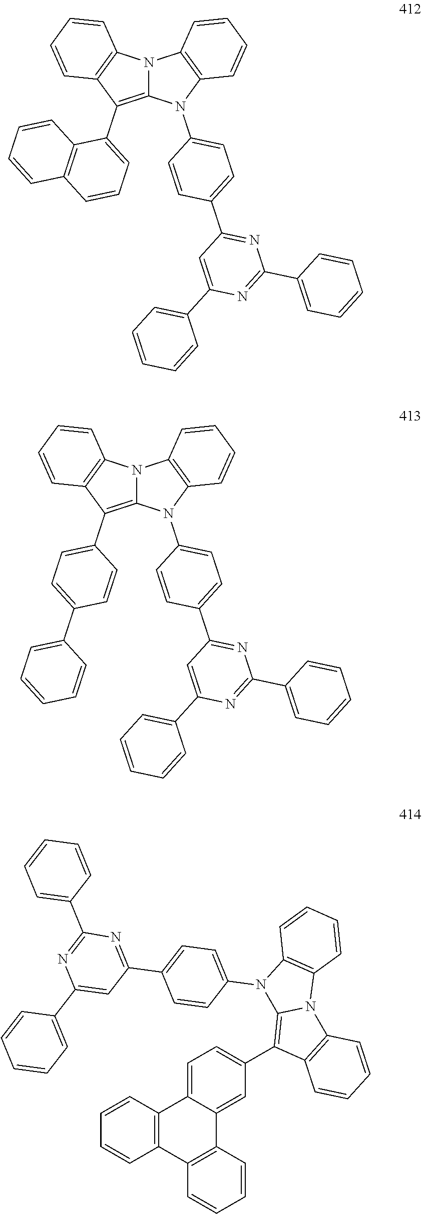 [SMILES: c1ccc(-c2cc(-c3ccc(-n4c5ccccc5n5c6ccccc6c(-c6cccc7ccccc67)c45)cc3)nc(-c3ccccc3)n2)cc1 ; c1ccc(-c2ccc(-c3c4ccccc4n4c5ccccc5n(-c5ccc(-c6cc(-c7ccccc7)nc(-c7ccccc7)n6)cc5)c34)cc2)cc1 ; c1ccc(-c2cc(-c3ccc(-n4c5ccccc5n5c6ccccc6c(-c6ccc7c8ccccc8c8ccccc8c7c6)c45)cc3)nc(-c3ccccc3)n2)cc1]

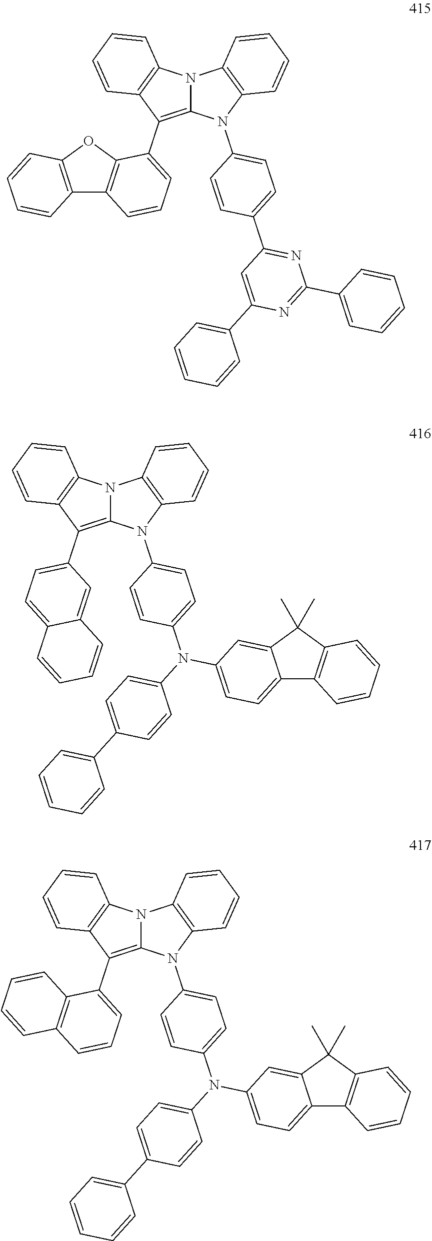 [SMILES: c1ccc(-c2cc(-c3ccc(-n4c5ccccc5n5c6ccccc6c(-c6cccc7c6oc6ccccc67)c45)cc3)nc(-c3ccccc3)n2)cc1 ; CC1(C)c2ccccc2-c2ccc(N(c3ccc(-c4ccccc4)cc3)c3ccc(-n4c5ccccc5n5c6ccccc6c(-c6ccc7ccccc7c6)c45)cc3)cc21 ; CC1(C)c2ccccc2-c2ccc(N(c3ccc(-c4ccccc4)cc3)c3ccc(-n4c5ccccc5n5c6ccccc6c(-c6cccc7ccccc67)c45)cc3)cc21]

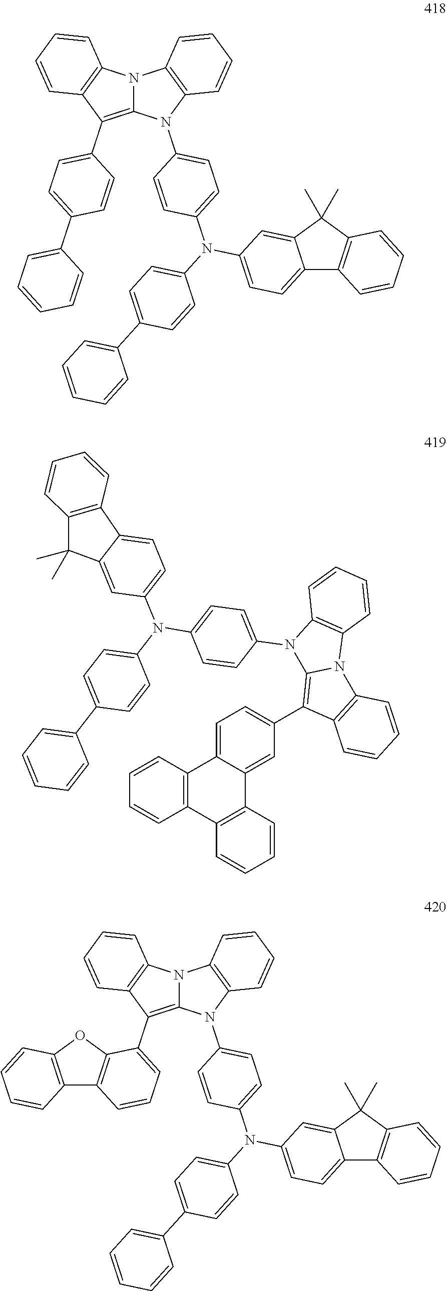 [SMILES: CC1(C)c2ccccc2-c2ccc(N(c3ccc(-c4ccccc4)cc3)c3ccc(-n4c5ccccc5n5c6ccccc6c(-c6ccc(-c7ccccc7)cc6)c45)cc3)cc21 ; CC1(C)c2ccccc2-c2ccc(N(c3ccc(-c4ccccc4)cc3)c3ccc(-n4c5ccccc5n5c6ccccc6c(-c6ccc7c8ccccc8c8ccccc8c7c6)c45)cc3)cc21 ; CC1(C)c2ccccc2-c2ccc(N(c3ccc(-c4ccccc4)cc3)c3ccc(-n4c5ccccc5n5c6ccccc6c(-c6cccc7c6oc6ccccc67)c45)cc3)cc21]

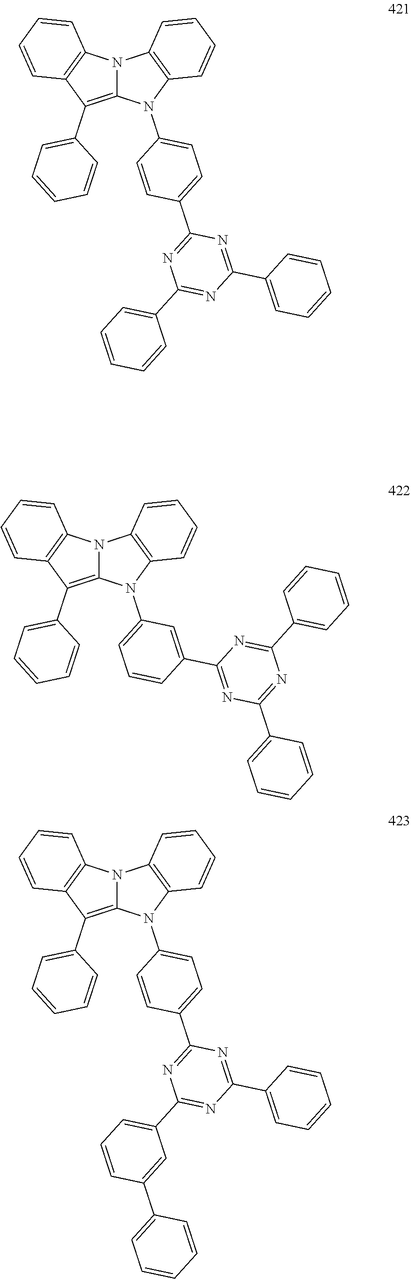 [SMILES: c1ccc(-c2nc(-c3ccccc3)nc(-c3ccc(-n4c5ccccc5n5c6ccccc6c(-c6ccccc6)c45)cc3)n2)cc1 ; c1ccc(-c2nc(-c3ccccc3)nc(-c3cccc(-n4c5ccccc5n5c6ccccc6c(-c6ccccc6)c45)c3)n2)cc1 ; c1ccc(-c2cccc(-c3nc(-c4ccccc4)nc(-c4ccc(-n5c6ccccc6n6c7ccccc7c(-c7ccccc7)c56)cc4)n3)c2)cc1]

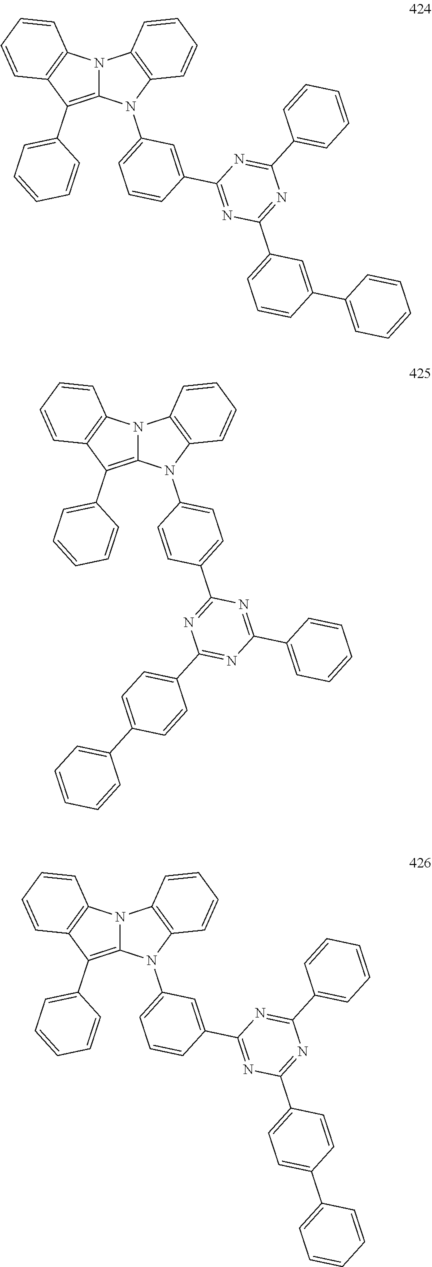 [SMILES: c1ccc(-c2cccc(-c3nc(-c4ccccc4)nc(-c4cccc(-n5c6ccccc6n6c7ccccc7c(-c7ccccc7)c56)c4)n3)c2)cc1 ; c1ccc(-c2ccc(-c3nc(-c4ccccc4)nc(-c4ccc(-n5c6ccccc6n6c7ccccc7c(-c7ccccc7)c56)cc4)n3)cc2)cc1 ; c1ccc(-c2ccc(-c3nc(-c4ccccc4)nc(-c4cccc(-n5c6ccccc6n6c7ccccc7c(-c7ccccc7)c56)c4)n3)cc2)cc1]

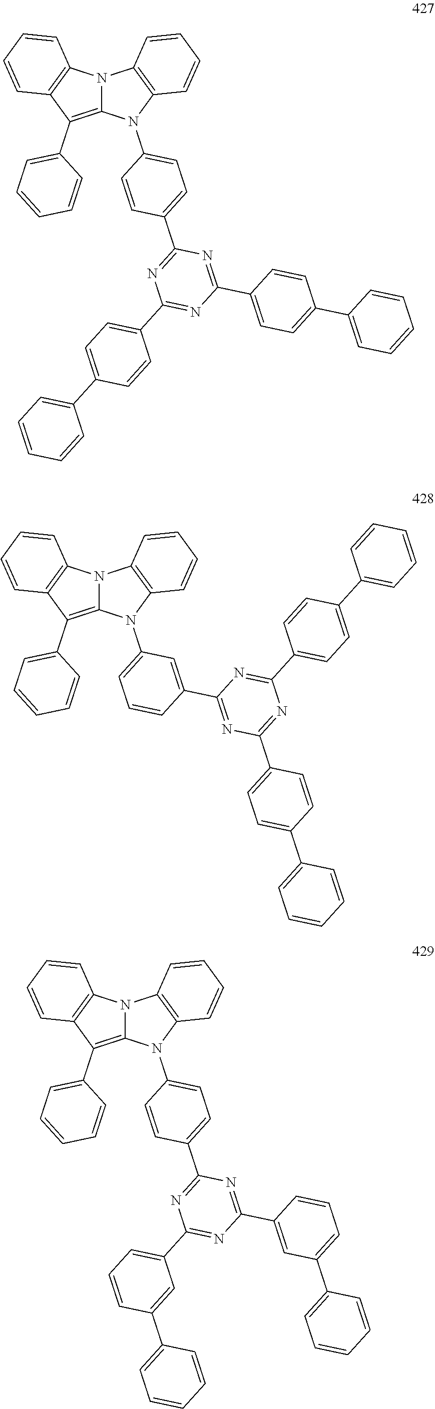 [SMILES: c1ccc(-c2ccc(-c3nc(-c4ccc(-c5ccccc5)cc4)nc(-c4ccc(-n5c6ccccc6n6c7ccccc7c(-c7ccccc7)c56)cc4)n3)cc2)cc1 ; c1ccc(-c2ccc(-c3nc(-c4ccc(-c5ccccc5)cc4)nc(-c4cccc(-n5c6ccccc6n6c7ccccc7c(-c7ccccc7)c56)c4)n3)cc2)cc1 ; c1ccc(-c2cccc(-c3nc(-c4ccc(-n5c6ccccc6n6c7ccccc7c(-c7ccccc7)c56)cc4)nc(-c4cccc(-c5ccccc5)c4)n3)c2)cc1]

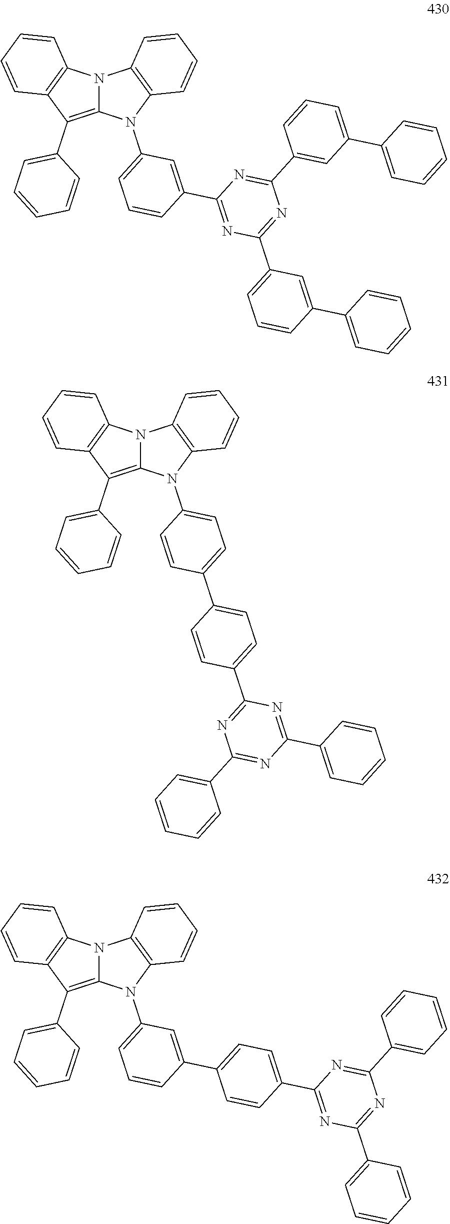 [SMILES: c1ccc(-c2cccc(-c3nc(-c4cccc(-c5ccccc5)c4)nc(-c4cccc(-n5c6ccccc6n6c7ccccc7c(-c7ccccc7)c56)c4)n3)c2)cc1 ; c1ccc(-c2nc(-c3ccccc3)nc(-c3ccc(-c4ccc(-n5c6ccccc6n6c7ccccc7c(-c7ccccc7)c56)cc4)cc3)n2)cc1 ; c1ccc(-c2nc(-c3ccccc3)nc(-c3ccc(-c4cccc(-n5c6ccccc6n6c7ccccc7c(-c7ccccc7)c56)c4)cc3)n2)cc1]

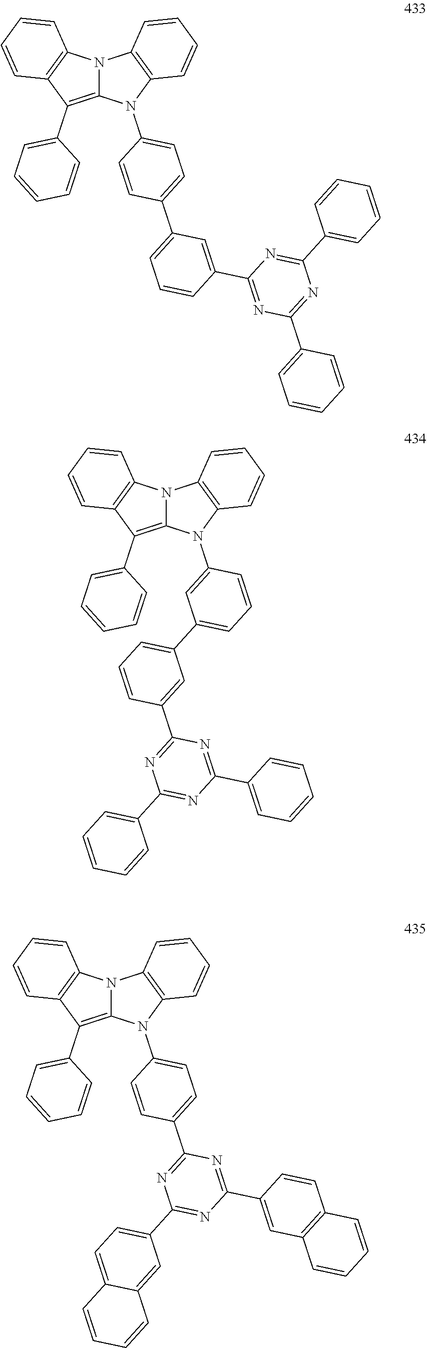 [SMILES: c1ccc(-c2nc(-c3ccccc3)nc(-c3cccc(-c4ccc(-n5c6ccccc6n6c7ccccc7c(-c7ccccc7)c56)cc4)c3)n2)cc1 ; c1ccc(-c2nc(-c3ccccc3)nc(-c3cccc(-c4cccc(-n5c6ccccc6n6c7ccccc7c(-c7ccccc7)c56)c4)c3)n2)cc1 ; c1ccc(-c2c3ccccc3n3c4ccccc4n(-c4ccc(-c5nc(-c6ccc7ccccc7c6)nc(-c6ccc7ccccc7c6)n5)cc4)c23)cc1]

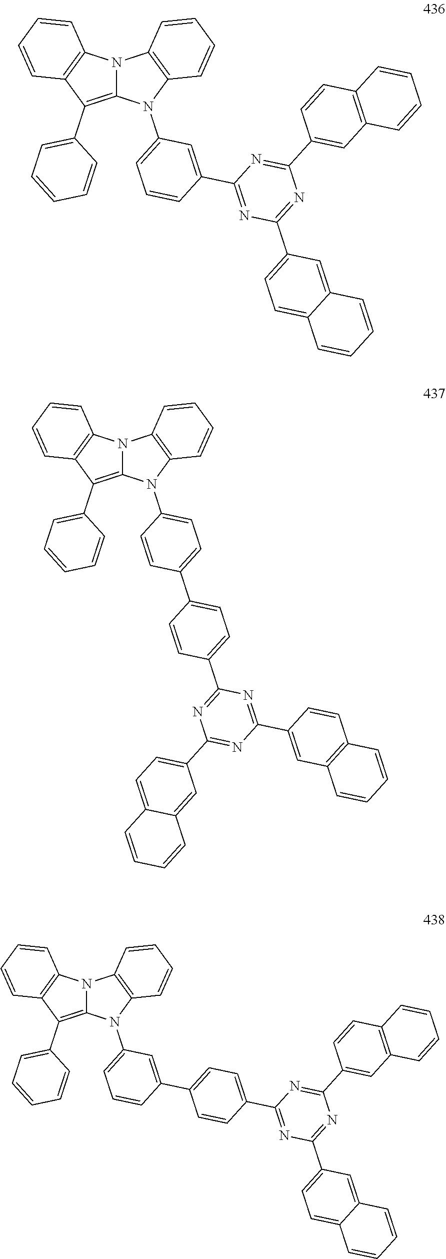 [SMILES: c1ccc(-c2c3ccccc3n3c4ccccc4n(-c4cccc(-c5nc(-c6ccc7ccccc7c6)nc(-c6ccc7ccccc7c6)n5)c4)c23)cc1 ; c1ccc(-c2c3ccccc3n3c4ccccc4n(-c4ccc(-c5ccc(-c6nc(-c7ccc8ccccc8c7)nc(-c7ccc8ccccc8c7)n6)cc5)cc4)c23)cc1 ; c1ccc(-c2c3ccccc3n3c4ccccc4n(-c4cccc(-c5ccc(-c6nc(-c7ccc8ccccc8c7)nc(-c7ccc8ccccc8c7)n6)cc5)c4)c23)cc1]

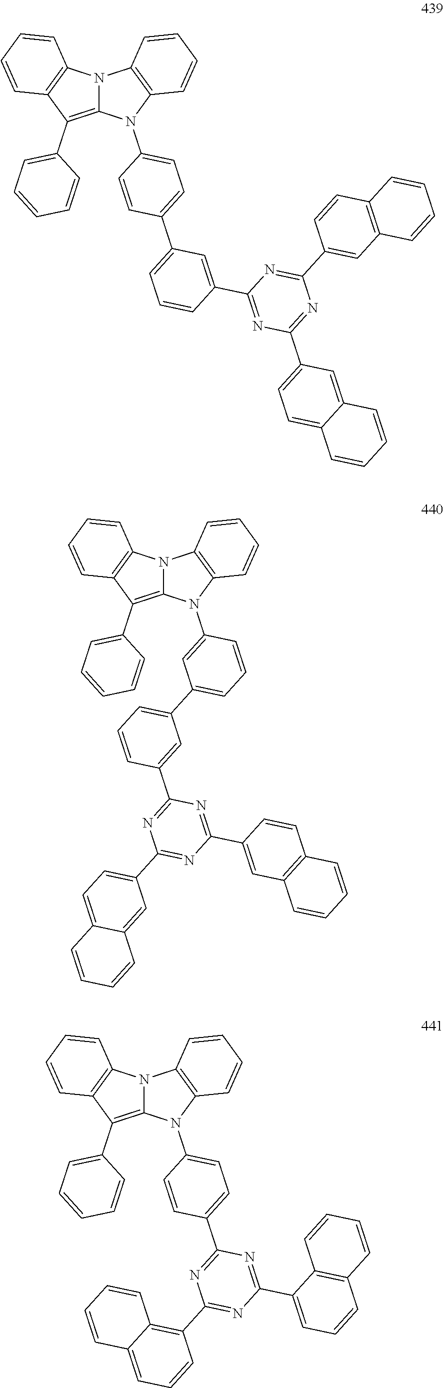 [SMILES: c1ccc(-c2c3ccccc3n3c4ccccc4n(-c4ccc(-c5cccc(-c6nc(-c7ccc8ccccc8c7)nc(-c7ccc8ccccc8c7)n6)c5)cc4)c23)cc1 ; c1ccc(-c2c3ccccc3n3c4ccccc4n(-c4cccc(-c5cccc(-c6nc(-c7ccc8ccccc8c7)nc(-c7ccc8ccccc8c7)n6)c5)c4)c23)cc1 ; c1ccc(-c2c3ccccc3n3c4ccccc4n(-c4ccc(-c5nc(-c6cccc7ccccc67)nc(-c6cccc7ccccc67)n5)cc4)c23)cc1]

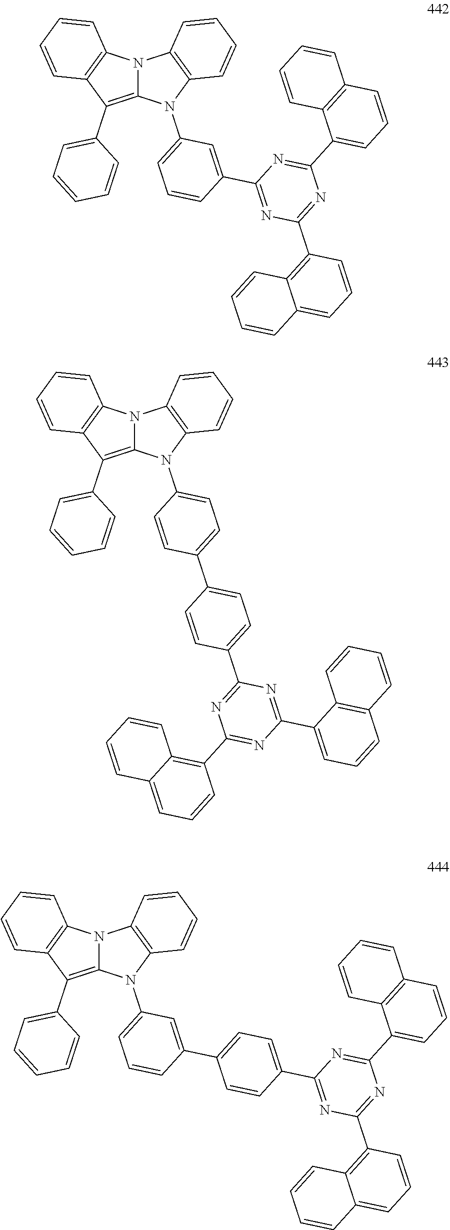 [SMILES: c1ccc(-c2c3ccccc3n3c4ccccc4n(-c4cccc(-c5nc(-c6cccc7ccccc67)nc(-c6cccc7ccccc67)n5)c4)c23)cc1 ; c1ccc(-c2c3ccccc3n3c4ccccc4n(-c4ccc(-c5ccc(-c6nc(-c7cccc8ccccc78)nc(-c7cccc8ccccc78)n6)cc5)cc4)c23)cc1 ; c1ccc(-c2c3ccccc3n3c4ccccc4n(-c4cccc(-c5ccc(-c6nc(-c7cccc8ccccc78)nc(-c7cccc8ccccc78)n6)cc5)c4)c23)cc1]

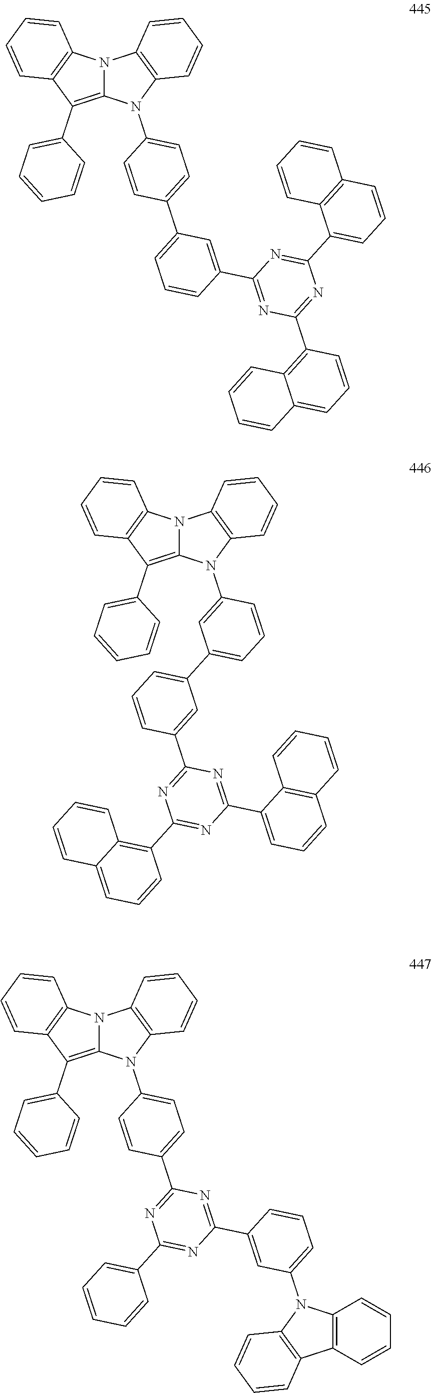 [SMILES: c1ccc(-c2c3ccccc3n3c4ccccc4n(-c4ccc(-c5cccc(-c6nc(-c7cccc8ccccc78)nc(-c7cccc8ccccc78)n6)c5)cc4)c23)cc1 ; c1ccc(-c2c3ccccc3n3c4ccccc4n(-c4cccc(-c5cccc(-c6nc(-c7cccc8ccccc78)nc(-c7cccc8ccccc78)n6)c5)c4)c23)cc1 ; c1ccc(-c2nc(-c3ccc(-n4c5ccccc5n5c6ccccc6c(-c6ccccc6)c45)cc3)nc(-c3cccc(-n4c5ccccc5c5ccccc54)c3)n2)cc1]

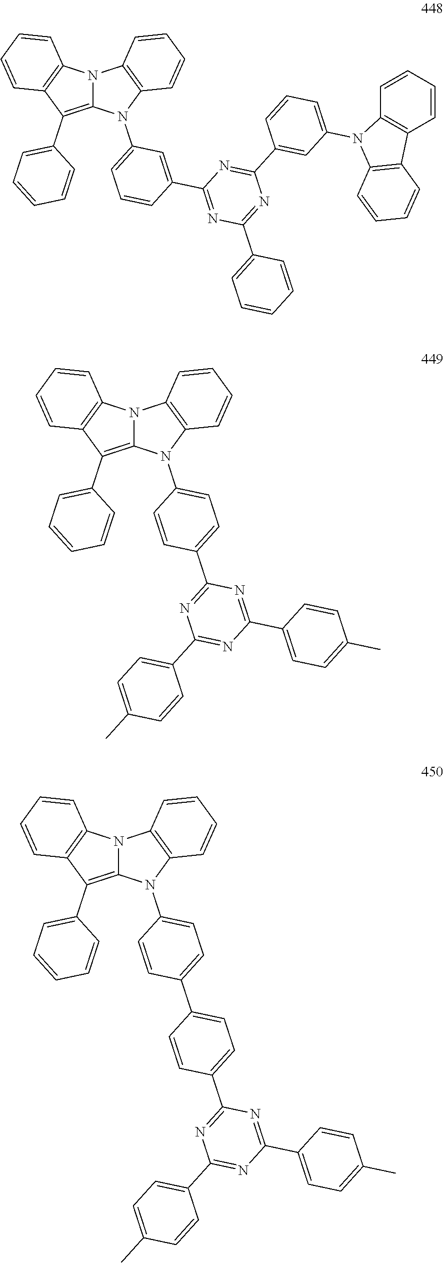 [SMILES: c1ccc(-c2nc(-c3cccc(-n4c5ccccc5c5ccccc54)c3)nc(-c3cccc(-n4c5ccccc5n5c6ccccc6c(-c6ccccc6)c45)c3)n2)cc1 ; Cc1ccc(-c2nc(-c3ccc(C)cc3)nc(-c3ccc(-n4c5ccccc5n5c6ccccc6c(-c6ccccc6)c45)cc3)n2)cc1 ; Cc1ccc(-c2nc(-c3ccc(C)cc3)nc(-c3ccc(-c4ccc(-n5c6ccccc6n6c7ccccc7c(-c7ccccc7)c56)cc4)cc3)n2)cc1]

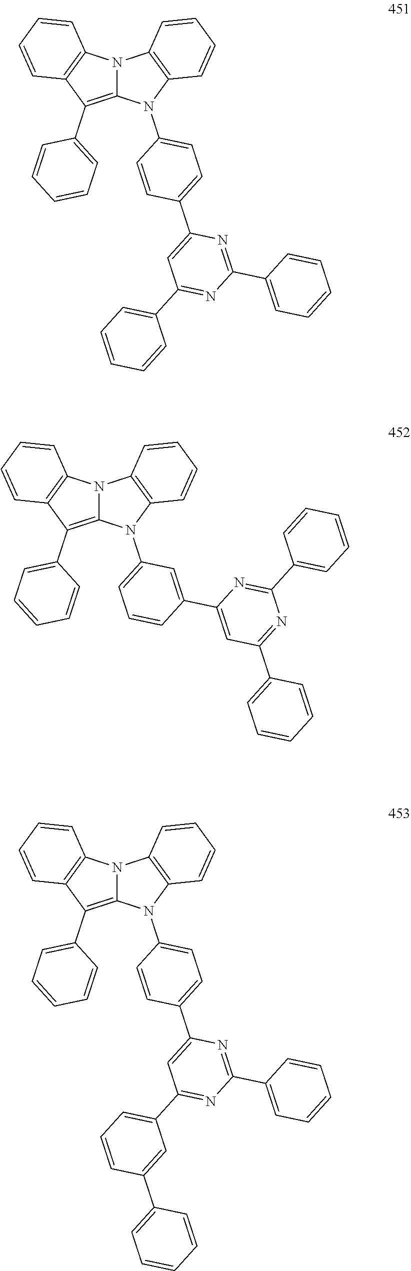 [SMILES: c1ccc(-c2cc(-c3ccc(-n4c5ccccc5n5c6ccccc6c(-c6ccccc6)c45)cc3)nc(-c3ccccc3)n2)cc1 ; c1ccc(-c2cc(-c3cccc(-n4c5ccccc5n5c6ccccc6c(-c6ccccc6)c45)c3)nc(-c3ccccc3)n2)cc1 ; c1ccc(-c2cccc(-c3cc(-c4ccc(-n5c6ccccc6n6c7ccccc7c(-c7ccccc7)c56)cc4)nc(-c4ccccc4)n3)c2)cc1]

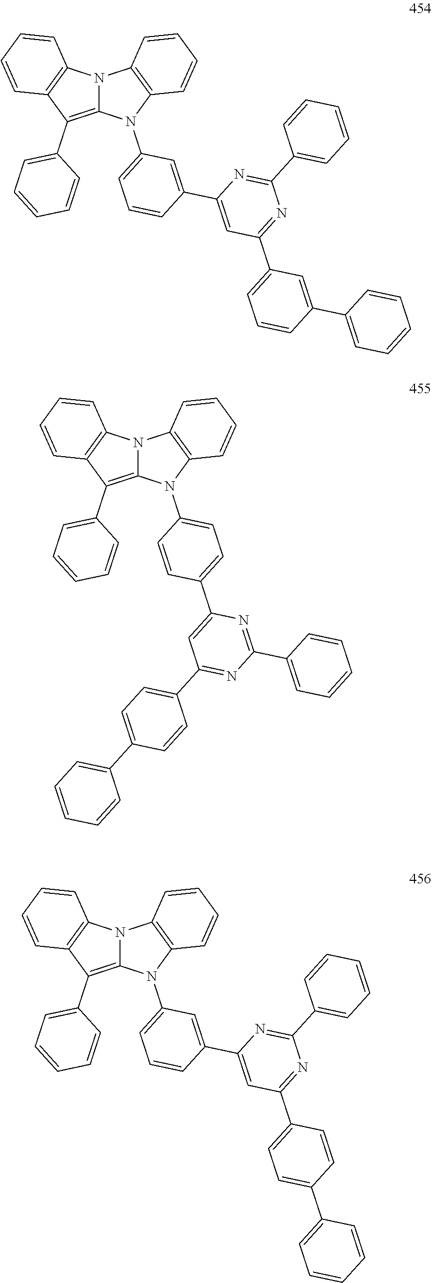 [SMILES: c1ccc(-c2cccc(-c3cc(-c4cccc(-n5c6ccccc6n6c7ccccc7c(-c7ccccc7)c56)c4)nc(-c4ccccc4)n3)c2)cc1 ; c1ccc(-c2ccc(-c3cc(-c4ccc(-n5c6ccccc6n6c7ccccc7c(-c7ccccc7)c56)cc4)nc(-c4ccccc4)n3)cc2)cc1 ; c1ccc(-c2ccc(-c3cc(-c4cccc(-n5c6ccccc6n6c7ccccc7c(-c7ccccc7)c56)c4)nc(-c4ccccc4)n3)cc2)cc1]

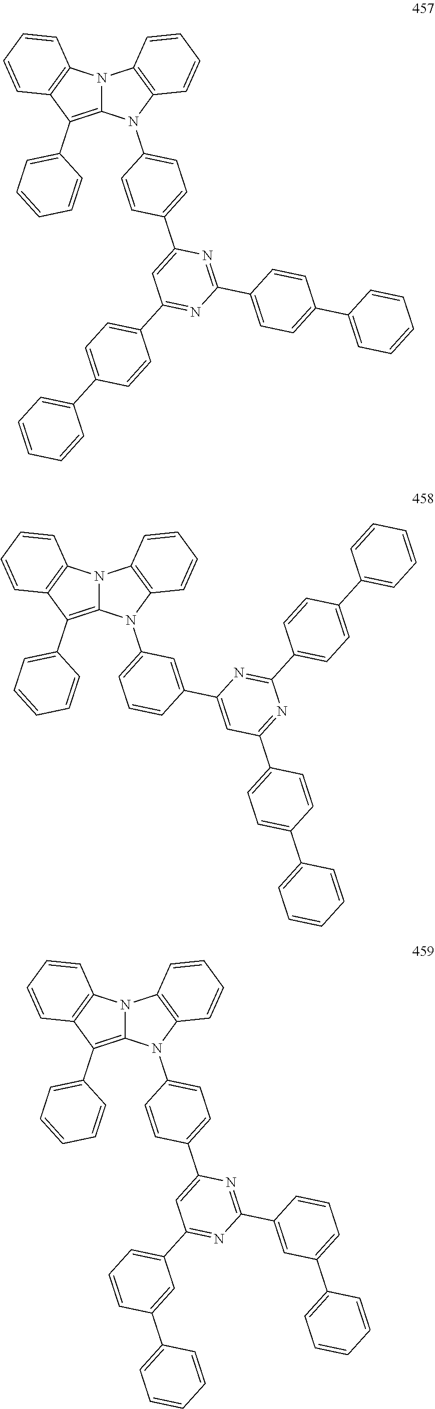 [SMILES: c1ccc(-c2ccc(-c3cc(-c4ccc(-n5c6ccccc6n6c7ccccc7c(-c7ccccc7)c56)cc4)nc(-c4ccc(-c5ccccc5)cc4)n3)cc2)cc1 ; c1ccc(-c2ccc(-c3cc(-c4cccc(-n5c6ccccc6n6c7ccccc7c(-c7ccccc7)c56)c4)nc(-c4ccc(-c5ccccc5)cc4)n3)cc2)cc1 ; c1ccc(-c2cccc(-c3cc(-c4ccc(-n5c6ccccc6n6c7ccccc7c(-c7ccccc7)c56)cc4)nc(-c4cccc(-c5ccccc5)c4)n3)c2)cc1]

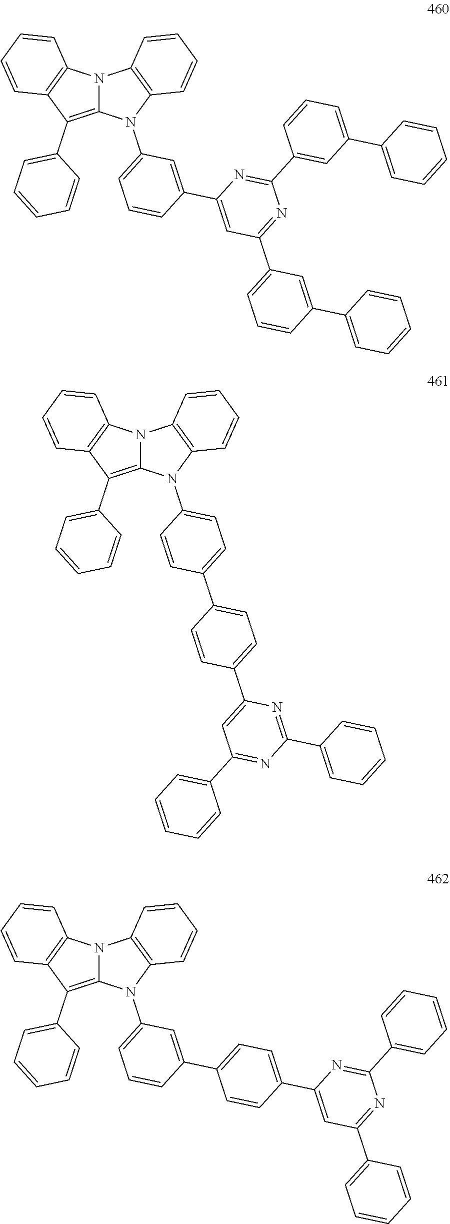 [SMILES: c1ccc(-c2cccc(-c3cc(-c4cccc(-n5c6ccccc6n6c7ccccc7c(-c7ccccc7)c56)c4)nc(-c4cccc(-c5ccccc5)c4)n3)c2)cc1 ; c1ccc(-c2cc(-c3ccc(-c4ccc(-n5c6ccccc6n6c7ccccc7c(-c7ccccc7)c56)cc4)cc3)nc(-c3ccccc3)n2)cc1 ; c1ccc(-c2cc(-c3ccc(-c4cccc(-n5c6ccccc6n6c7ccccc7c(-c7ccccc7)c56)c4)cc3)nc(-c3ccccc3)n2)cc1]

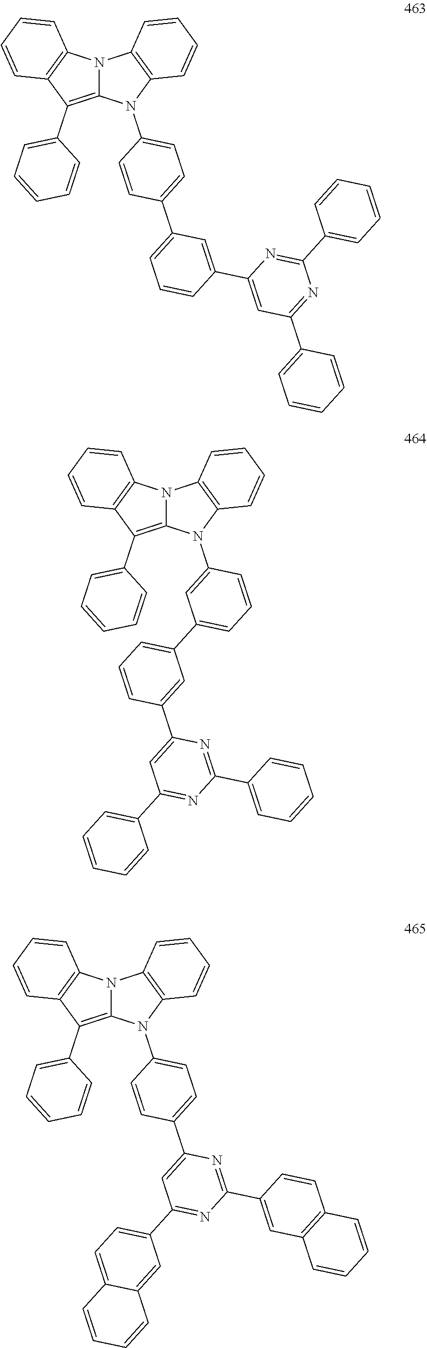 [SMILES: c1ccc(-c2cc(-c3cccc(-c4ccc(-n5c6ccccc6n6c7ccccc7c(-c7ccccc7)c56)cc4)c3)nc(-c3ccccc3)n2)cc1 ; c1ccc(-c2cc(-c3cccc(-c4cccc(-n5c6ccccc6n6c7ccccc7c(-c7ccccc7)c56)c4)c3)nc(-c3ccccc3)n2)cc1 ; c1ccc(-c2c3ccccc3n3c4ccccc4n(-c4ccc(-c5cc(-c6ccc7ccccc7c6)nc(-c6ccc7ccccc7c6)n5)cc4)c23)cc1]

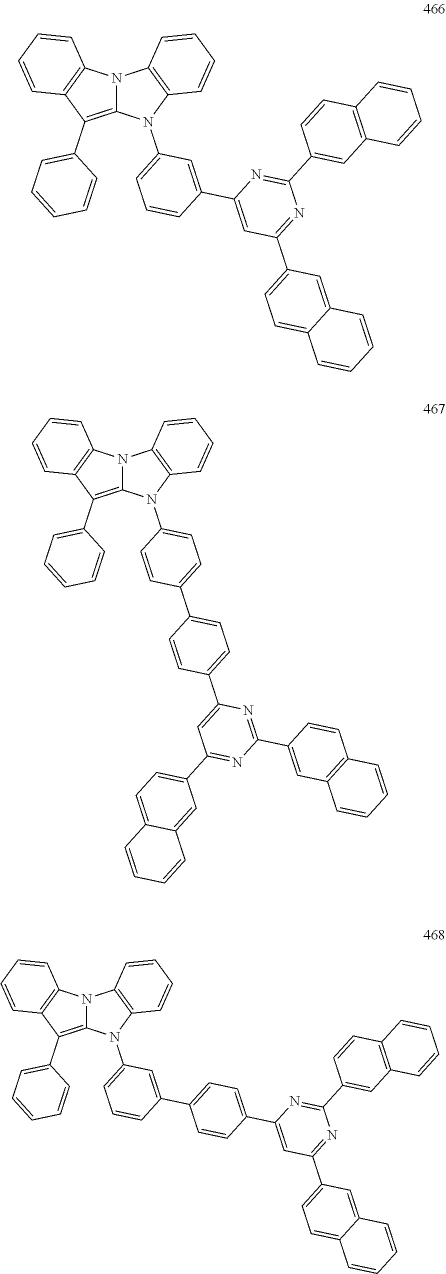 [SMILES: c1ccc(-c2c3ccccc3n3c4ccccc4n(-c4cccc(-c5cc(-c6ccc7ccccc7c6)nc(-c6ccc7ccccc7c6)n5)c4)c23)cc1 ; c1ccc(-c2c3ccccc3n3c4ccccc4n(-c4ccc(-c5ccc(-c6cc(-c7ccc8ccccc8c7)nc(-c7ccc8ccccc8c7)n6)cc5)cc4)c23)cc1 ; c1ccc(-c2c3ccccc3n3c4ccccc4n(-c4cccc(-c5ccc(-c6cc(-c7ccc8ccccc8c7)nc(-c7ccc8ccccc8c7)n6)cc5)c4)c23)cc1]

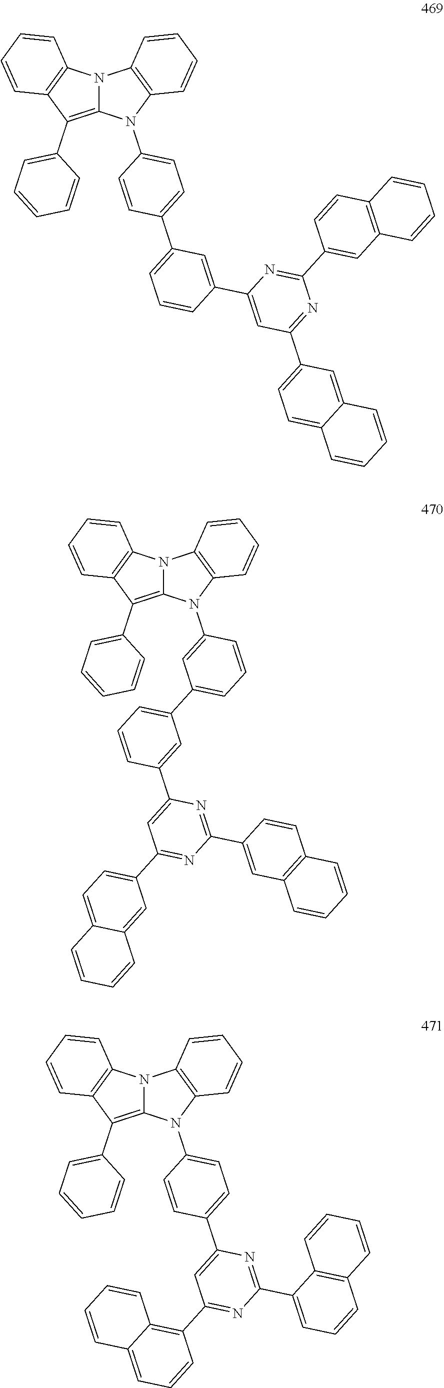 [SMILES: c1ccc(-c2c3ccccc3n3c4ccccc4n(-c4ccc(-c5cccc(-c6cc(-c7ccc8ccccc8c7)nc(-c7ccc8ccccc8c7)n6)c5)cc4)c23)cc1 ; c1ccc(-c2c3ccccc3n3c4ccccc4n(-c4cccc(-c5cccc(-c6cc(-c7ccc8ccccc8c7)nc(-c7ccc8ccccc8c7)n6)c5)c4)c23)cc1 ; c1ccc(-c2c3ccccc3n3c4ccccc4n(-c4ccc(-c5cc(-c6cccc7ccccc67)nc(-c6cccc7ccccc67)n5)cc4)c23)cc1]

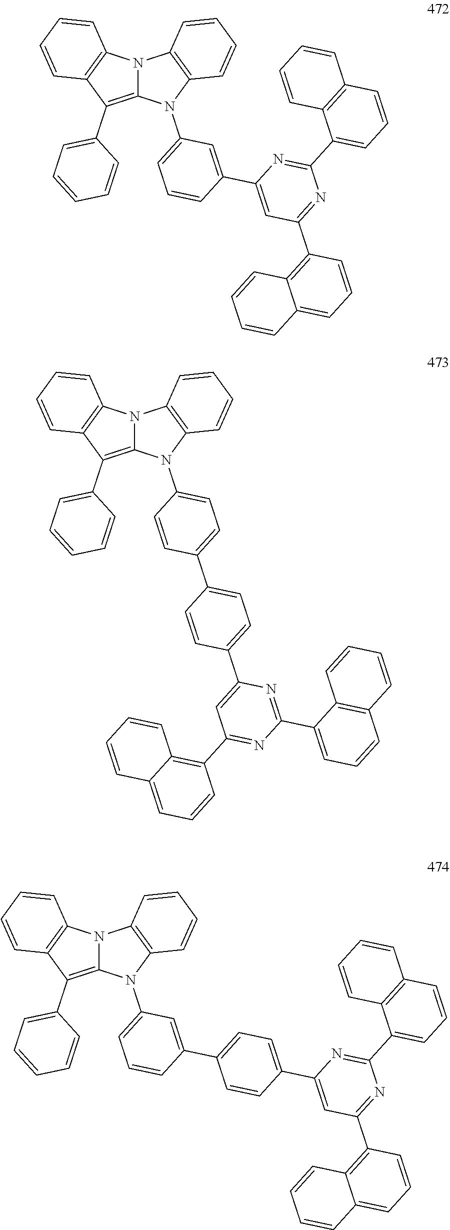 [SMILES: c1ccc(-c2c3ccccc3n3c4ccccc4n(-c4cccc(-c5cc(-c6cccc7ccccc67)nc(-c6cccc7ccccc67)n5)c4)c23)cc1 ; c1ccc(-c2c3ccccc3n3c4ccccc4n(-c4ccc(-c5ccc(-c6cc(-c7cccc8ccccc78)nc(-c7cccc8ccccc78)n6)cc5)cc4)c23)cc1 ; c1ccc(-c2c3ccccc3n3c4ccccc4n(-c4cccc(-c5ccc(-c6cc(-c7cccc8ccccc78)nc(-c7cccc8ccccc78)n6)cc5)c4)c23)cc1]

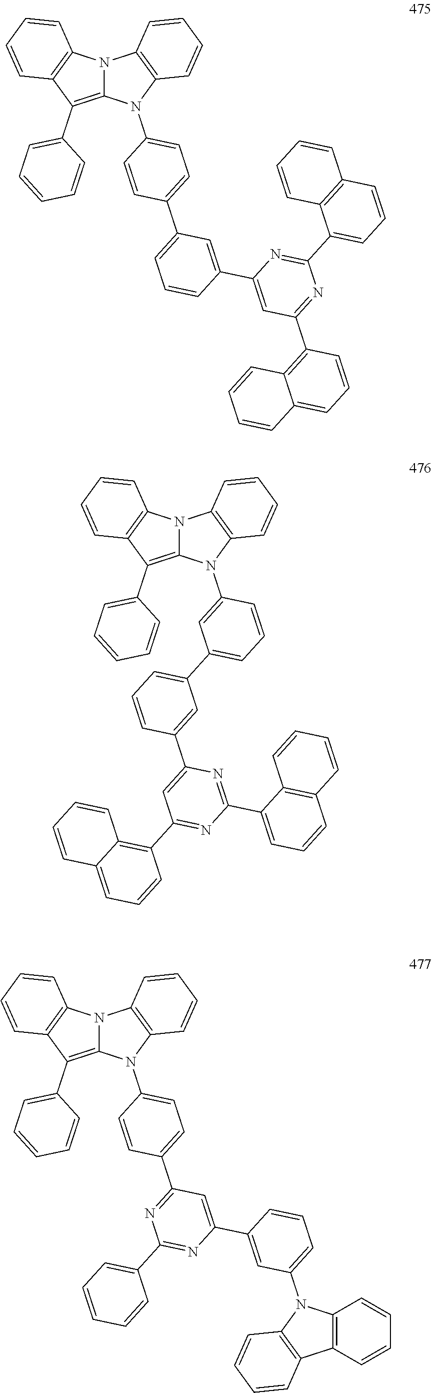 [SMILES: c1ccc(-c2c3ccccc3n3c4ccccc4n(-c4ccc(-c5cccc(-c6cc(-c7cccc8ccccc78)nc(-c7cccc8ccccc78)n6)c5)cc4)c23)cc1 ; c1ccc(-c2c3ccccc3n3c4ccccc4n(-c4cccc(-c5cccc(-c6cc(-c7cccc8ccccc78)nc(-c7cccc8ccccc78)n6)c5)c4)c23)cc1 ; c1ccc(-c2nc(-c3ccc(-n4c5ccccc5n5c6ccccc6c(-c6ccccc6)c45)cc3)cc(-c3cccc(-n4c5ccccc5c5ccccc54)c3)n2)cc1]

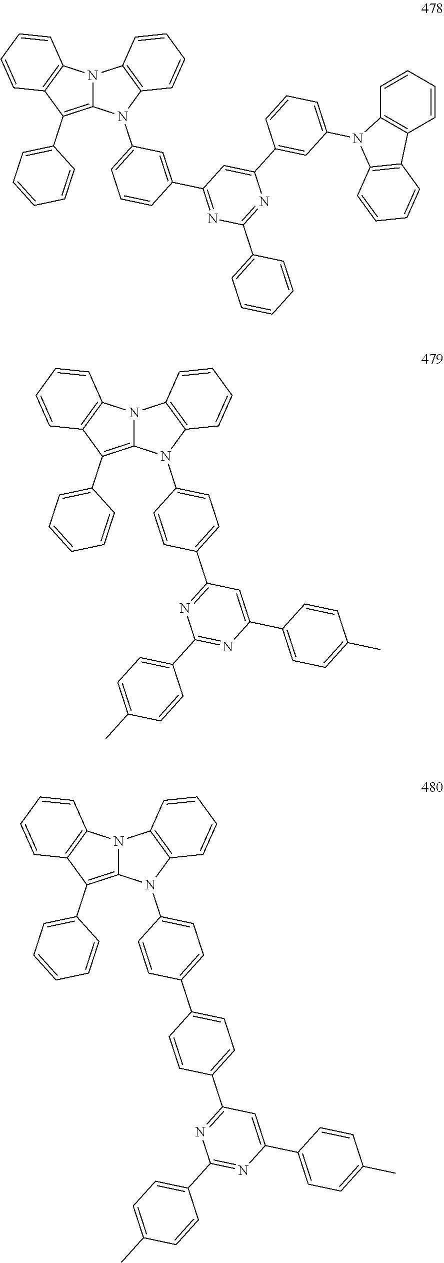 [SMILES: c1ccc(-c2nc(-c3cccc(-n4c5ccccc5c5ccccc54)c3)cc(-c3cccc(-n4c5ccccc5n5c6ccccc6c(-c6ccccc6)c45)c3)n2)cc1 ; Cc1ccc(-c2cc(-c3ccc(-n4c5ccccc5n5c6ccccc6c(-c6ccccc6)c45)cc3)nc(-c3ccc(C)cc3)n2)cc1 ; Cc1ccc(-c2cc(-c3ccc(-c4ccc(-n5c6ccccc6n6c7ccccc7c(-c7ccccc7)c56)cc4)cc3)nc(-c3ccc(C)cc3)n2)cc1]

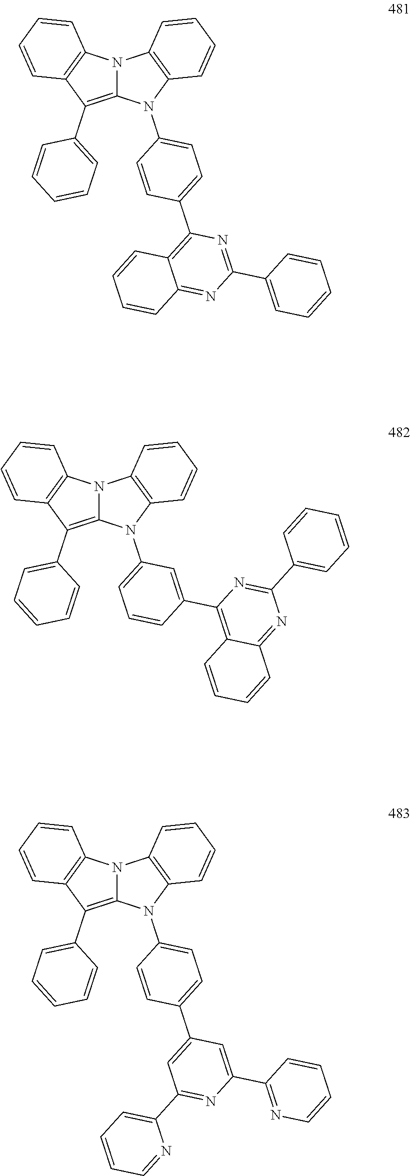 [SMILES: c1ccc(-c2nc(-c3ccc(-n4c5ccccc5n5c6ccccc6c(-c6ccccc6)c45)cc3)c3ccccc3n2)cc1 ; c1ccc(-c2nc(-c3cccc(-n4c5ccccc5n5c6ccccc6c(-c6ccccc6)c45)c3)c3ccccc3n2)cc1 ; c1ccc(-c2c3ccccc3n3c4ccccc4n(-c4ccc(-c5cc(-c6ccccn6)nc(-c6ccccn6)c5)cc4)c23)cc1]

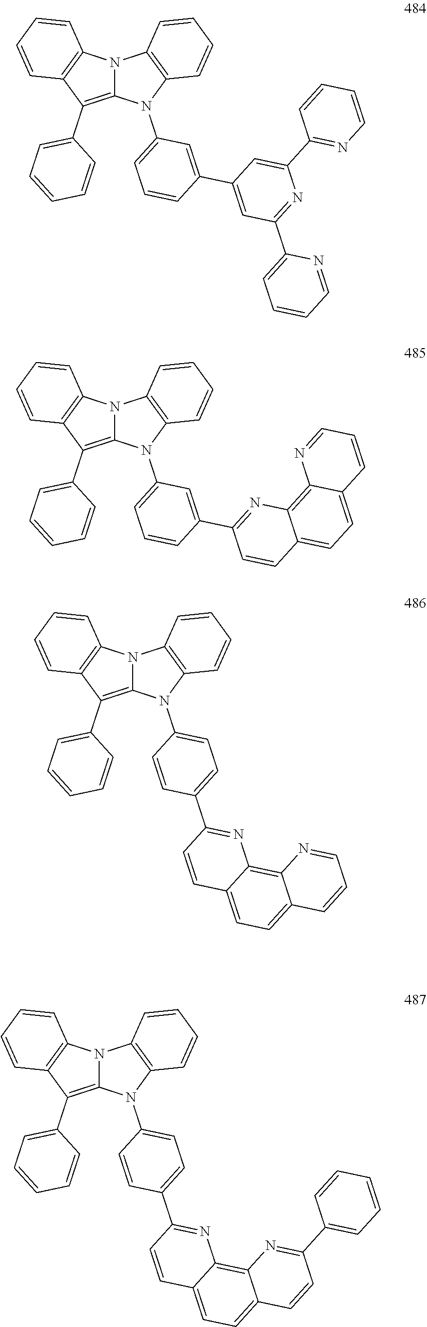 [SMILES: c1ccc(-c2c3ccccc3n3c4ccccc4n(-c4cccc(-c5cc(-c6ccccn6)nc(-c6ccccn6)c5)c4)c23)cc1 ; c1ccc(-c2c3ccccc3n3c4ccccc4n(-c4cccc(-c5ccc6ccc7cccnc7c6n5)c4)c23)cc1 ; c1ccc(-c2c3ccccc3n3c4ccccc4n(-c4ccc(-c5ccc6ccc7cccnc7c6n5)cc4)c23)cc1 ; c1ccc(-c2ccc3ccc4ccc(-c5ccc(-n6c7ccccc7n7c8ccccc8c(-c8ccccc8)c67)cc5)nc4c3n2)cc1]

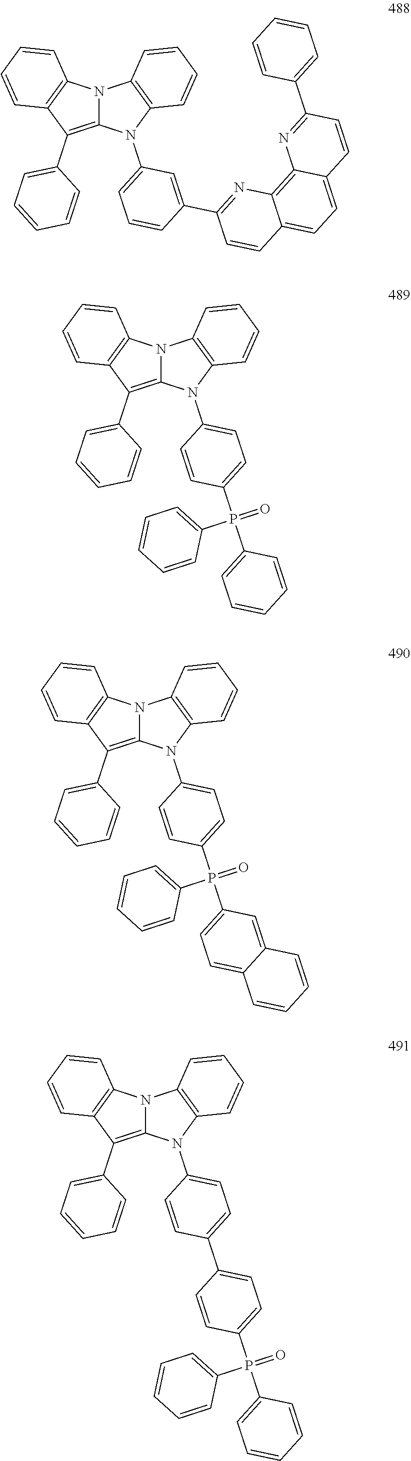 [SMILES: c1ccc(-c2ccc3ccc4ccc(-c5cccc(-n6c7ccccc7n7c8ccccc8c(-c8ccccc8)c67)c5)nc4c3n2)cc1 ; O=P(c1ccccc1)(c1ccccc1)c1ccc(-n2c3ccccc3n3c4ccccc4c(-c4ccccc4)c23)cc1 ; O=P(c1ccccc1)(c1ccc(-n2c3ccccc3n3c4ccccc4c(-c4ccccc4)c23)cc1)c1ccc2ccccc2c1 ; O=P(c1ccccc1)(c1ccccc1)c1ccc(-c2ccc(-n3c4ccccc4n4c5ccccc5c(-c5ccccc5)c34)cc2)cc1]

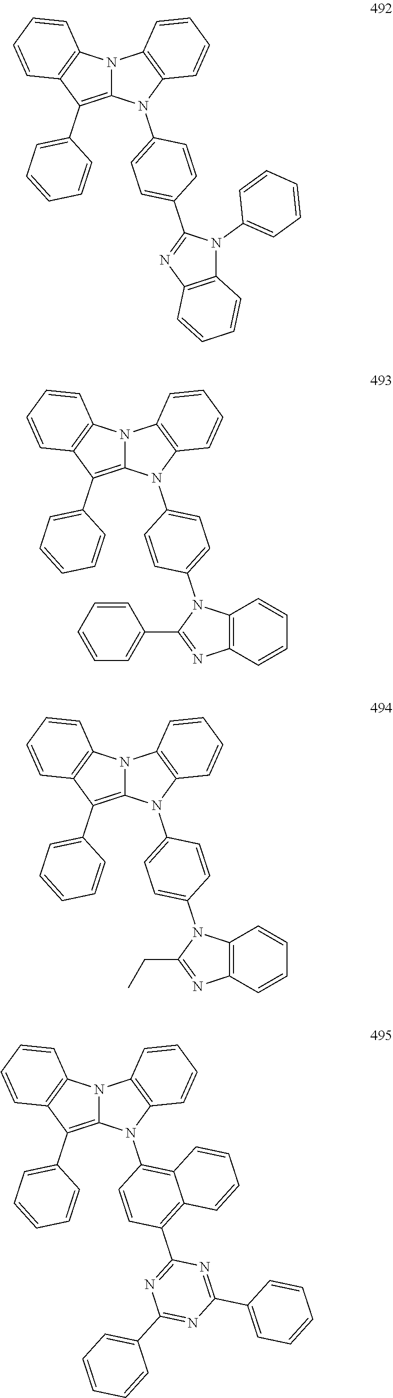 [SMILES: c1ccc(-c2c3ccccc3n3c4ccccc4n(-c4ccc(-c5nc6ccccc6n5-c5ccccc5)cc4)c23)cc1 ; c1ccc(-c2c3ccccc3n3c4ccccc4n(-c4ccc(-n5c(-c6ccccc6)nc6ccccc65)cc4)c23)cc1 ; CCc1nc2ccccc2n1-c1ccc(-n2c3ccccc3n3c4ccccc4c(-c4ccccc4)c23)cc1 ; c1ccc(-c2nc(-c3ccccc3)nc(-c3ccc(-n4c5ccccc5n5c6ccccc6c(-c6ccccc6)c45)c4ccccc34)n2)cc1]

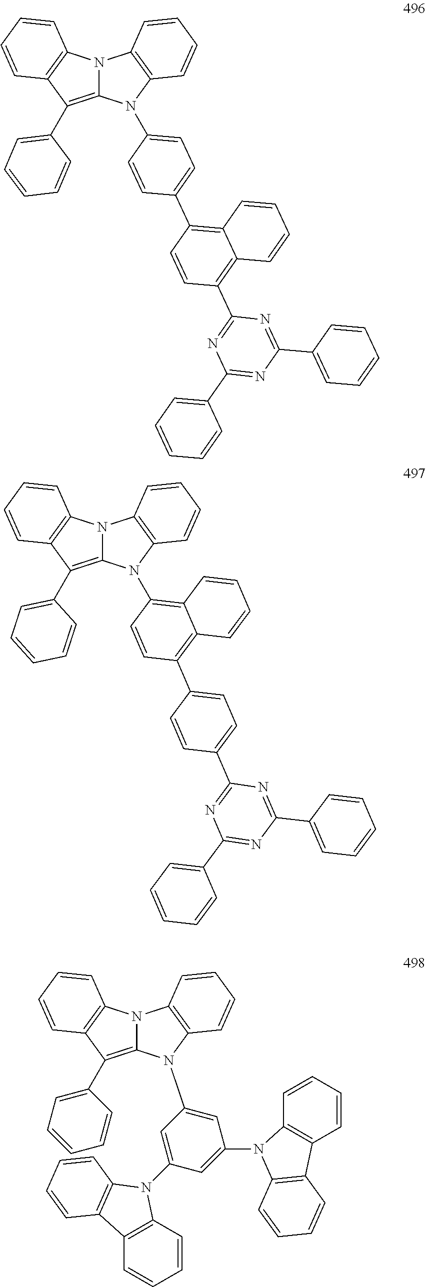

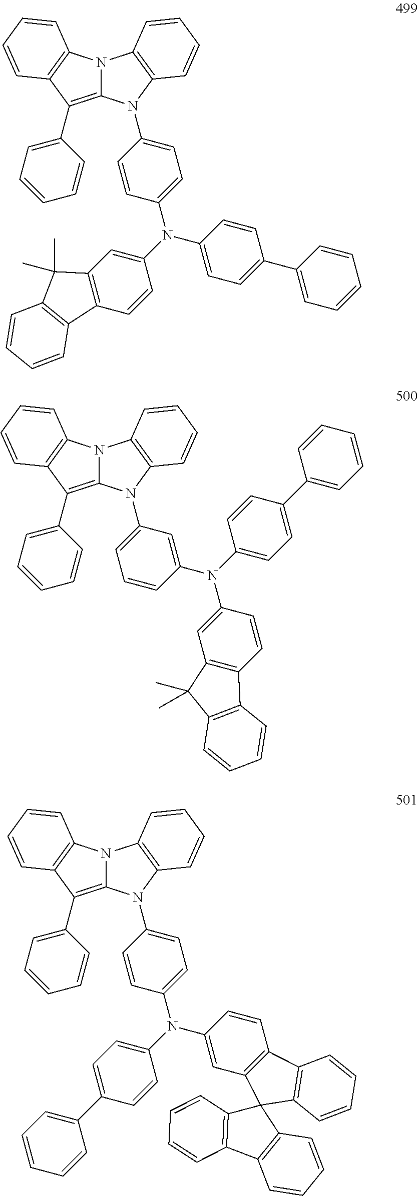

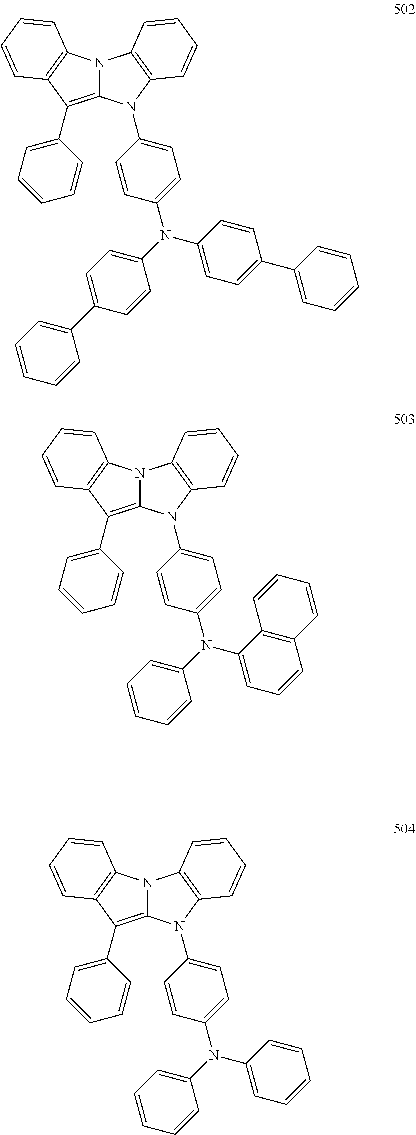

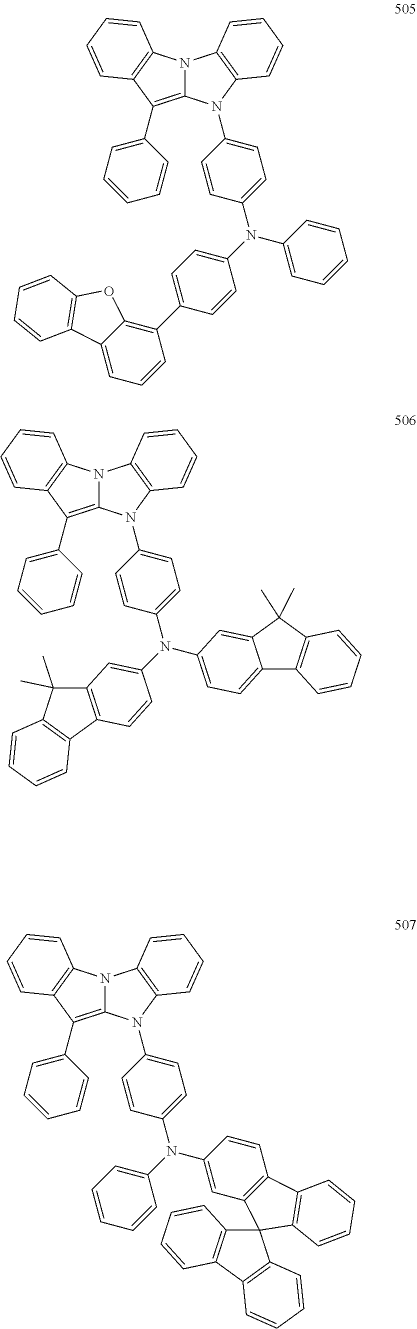 [SMILES: c1ccc(-c2c3ccccc3n3c4ccccc4n(-c4ccc(N(c5ccccc5)c5ccc(-c6cccc7c6oc6ccccc67)cc5)cc4)c23)cc1 ; CC1(C)c2ccccc2-c2ccc(N(c3ccc(-n4c5ccccc5n5c6ccccc6c(-c6ccccc6)c45)cc3)c3ccc4c(c3)C(C)(C)c3ccccc3-4)cc21 ; c1ccc(-c2c3ccccc3n3c4ccccc4n(-c4ccc(N(c5ccccc5)c5ccc6c(c5)C5(c7ccccc7-c7ccccc75)c5ccccc5-6)cc4)c23)cc1]

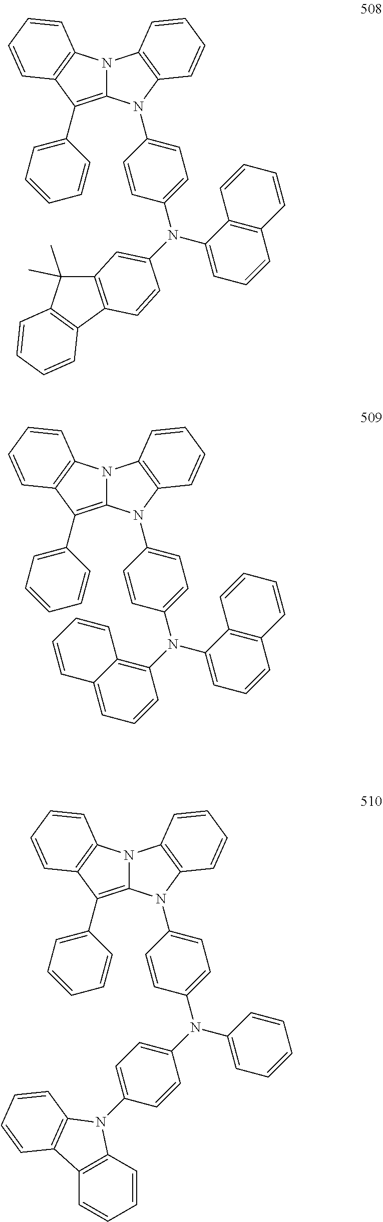 [SMILES: CC1(C)c2ccccc2-c2ccc(N(c3ccc(-n4c5ccccc5n5c6ccccc6c(-c6ccccc6)c45)cc3)c3cccc4ccccc34)cc21 ; c1ccc(-c2c3ccccc3n3c4ccccc4n(-c4ccc(N(c5cccc6ccccc56)c5cccc6ccccc56)cc4)c23)cc1 ; c1ccc(-c2c3ccccc3n3c4ccccc4n(-c4ccc(N(c5ccccc5)c5ccc(-n6c7ccccc7c7ccccc76)cc5)cc4)c23)cc1]

By introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

Meanwhile, the compound has a high glass transition temperature (Tg) and thereby has superior thermal stability. Such an increase in the thermal stability becomes an important factor in providing driving stability to a device.

In addition, one embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

In one embodiment of the present application, the first electrode may be an anode, and the second electrode may be a cathode.

In another embodiment, the first electrode may be a cathode, and the second electrode may be an anode.

Specific descriptions on the heterocyclic compound represented by Chemical Formula 1 are the same as the descriptions provided above.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the heterocyclic compound according to Chemical Formula 1 may be used as a material of the green organic light emitting device.

In one embodiment of the present application, the organic light emitting device may be a red organic light emitting device, and the heterocyclic compound according to Chemical Formula 1 may be used as a material of the red organic light emitting device.

The organic light emitting device of the present disclosure may be manufactured using common organic light emitting device manufacturing methods and materials except that one or more of the organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may be formed in a multilayer structure in which two or more organic material layers are laminated. For example, the organic light emitting device of the present disclosure may have a structure comprising a hole injection layer, a hole transfer layer, a light emitting layer, an electron transfer layer, an electron injection layer and the like as the organic material layer. However, the structure of the organic light emitting device is not limited thereto, and may comprise a smaller number of organic material layers.

In the organic light emitting device of the present disclosure, the organic material layer comprises an electron injection layer or an electron transfer layer, and the electron injection layer or the electron transfer layer may comprise the heterocyclic compound.

In the organic light emitting device of the present disclosure, the organic material layer comprises an electron transfer layer, and the electron transfer layer may comprise the heterocyclic compound.

In another organic light emitting device, the organic material layer comprises an electron blocking layer or a hole blocking layer, and the electron blocking layer or the hole blocking layer may comprise the heterocyclic compound.

In another organic light emitting device, the organic material layer comprises a hole blocking layer, and the hole blocking layer may comprise the heterocyclic compound.

In the organic light emitting device provided in one embodiment of the present application, the organic material layer comprises a hole injection layer, and the hole injection layer comprises the heterocyclic compound.

In another organic light emitting device, the organic material layer comprises a hole transfer layer, and the hole transfer layer may comprise the heterocyclic compound.

In another organic light emitting device, the organic material layer comprises an electron transfer layer, a light emitting layer or a hole blocking layer, and the electron transfer layer, the light emitting layer or the hole blocking layer may comprise the heterocyclic compound.

The organic light emitting device of the present disclosure may further comprise one, two or more layers selected from the group consisting of a light emitting layer, a hole injection layer, a hole transfer layer, an electron injection layer, an electron transfer layer, an electron blocking layer and a hole blocking layer.

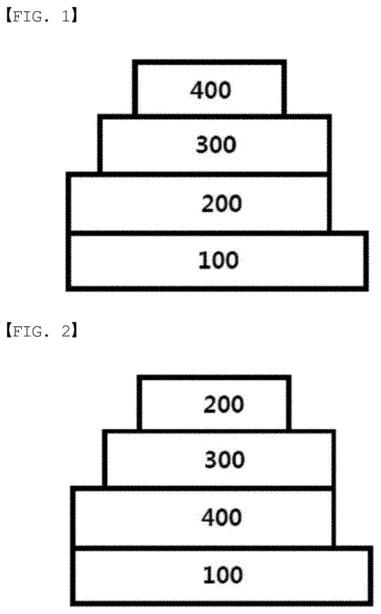

FIG. 1 to FIG. 4 illustrate a lamination order of electrodes and organic material layers of an organic light emitting device according to one embodiment of the present application. However, the scope of the present application is not limited to these diagrams, and structures of organic light emitting devices known in the art may also be used in the present application.

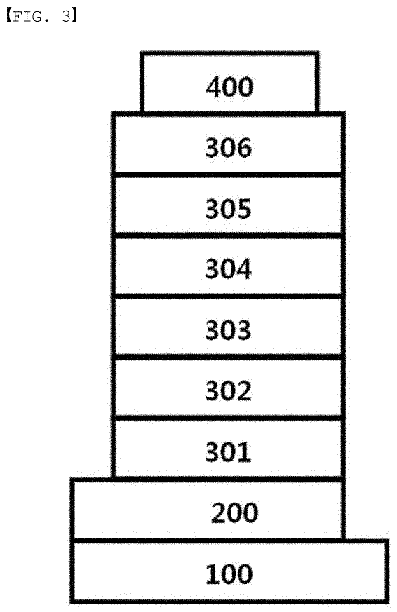

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

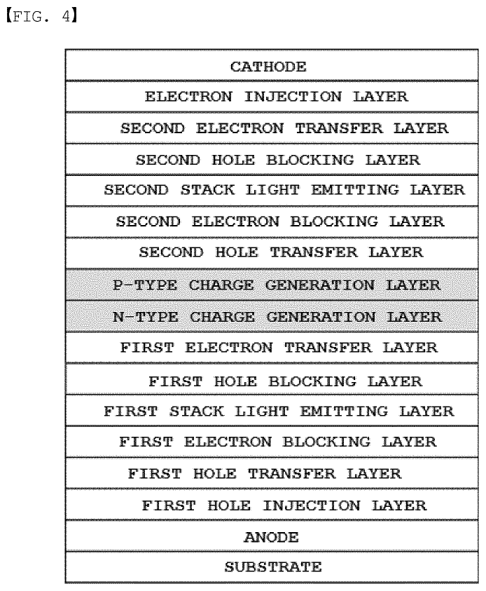

FIG. 3 illustrates a case of the organic material layer being a multilayer. The organic light emitting device according to FIG. 3 comprises a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, layers other than the light emitting layer may not be included, and other necessary functional layers may be further added.

The organic material layer comprising Chemical Formula 1 may further comprise other materials as necessary.

In addition, the organic light emitting device according to one embodiment of the present application comprises an anode, a cathode, and two or more stacks provided between the anode and the cathode, the two or more stacks each independently comprise a light emitting layer, a charge generation layer is included between the two or more stacks, and the charge generation layer comprises the compound represented by Chemical Formula 1.

In addition, the organic light emitting device according to one embodiment of the present application comprises an anode, a first stack provided on the anode and comprising a first light emitting layer, a charge generation layer provided on the first stack, a second stack provided on the charge generation layer and comprising a second light emitting layer, and a cathode provided on the second stack. Herein, the charge generation layer may comprise the heterocyclic compound represented by Chemical Formula 1. In addition, the first stack and the second stack may each independently further comprise one or more types of the hole injection layer, the hole transfer layer, the hole blocking layer, the electron transfer layer, the electron injection layer and the like described above.

The charge generation layer may be an N-type charge generation layer, and the charge generation layer may further comprise a dopant known in the art in addition to the heterocyclic compound represented by Chemical Formula 1.

As the organic light emitting device according to one embodiment of the present application, an organic light emitting device having a 2-stack tandem structure is schematically illustrated in FIG. 4.

Herein, the first electron blocking layer, the first hole blocking layer, the second hole blocking layer and the like described in FIG. 4 may not be included in some cases.

In the organic light emitting device according to one embodiment of the present application, materials other than the compound of Chemical Formula 1 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material comprise metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material comprise metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrene-sulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed.

For example, any two or more types of materials among n-type host materials or p-type host materials may be selected and used as a host material of a light emitting layer.

The organic light emitting device according to one embodiment of the present application may be a top-emission type, a bottom-emission type or a dual-emission type depending on the materials used.

The heterocyclic compound according to one embodiment of the present application may also be used in an organic electronic device comprising an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### PREPARATION EXAMPLE

### [Preparation Example 1] Preparation of Intermediate A1

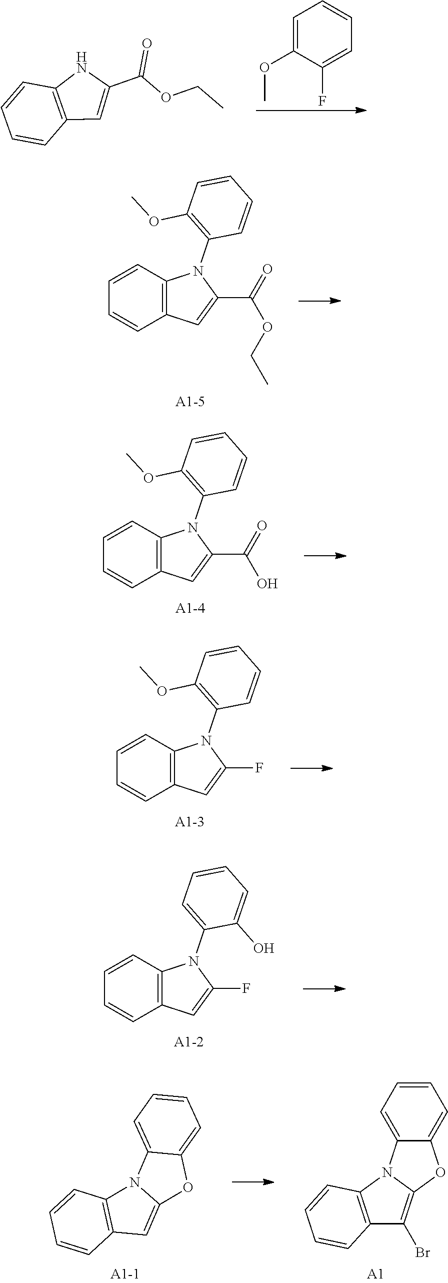

### Preparation of Intermediate A1-5

Ethyl 1H-indole-2-carboxylate (50 g, 264.26 mmol), 1-fluoro-2-methoxybenzene (36.66 g, 290.68 mmol), Cs₂CO₃ (258.3 g, 792.77 mmol) and dimethylacetamide (500 ml) were introduced into a one-neck round bottom flask, and stirred for 12 hours under reflux. The result was cooled and then filtered, and after removing the solvent, the result was purified by column chromatography using dichloromethane and hexane as a developing solvent to obtain Intermediate A1-5 (70 g, 90%).

### Preparation of Intermediate A1-4

Intermediate A1-5 (70 g, 237.02 mmol), NaOH (94.81 g, 2370.23 mmol), tetrahydrofuran (THF) (700 ml) and H₂O (1400 ml) were introduced into a one-neck round bottom flask, and stirred for 5 hours under reflux. After the reaction was completed, the result was extracted with 1N HCl and H₂O, and after removing the solvent, the result was purified by column chromatography using dichloromethane and hexane as a developing solvent to obtain Intermediate A1-4 (50 g, 79%).

### Preparation of Intermediate A1-3

Intermediate A1-4 (50 g, 187.07 mmol), KF (43.48 g, 748.28 mmol), selectfluor (132.54 g, 374.14 mmol) 1,2-dichloroethane (DCE) (200 ml) and H₂O (100 ml) were introduced into a one-neck round bottom flask, and stirred for 15 hours under reflux. After the reaction was completed, the result was extracted with methylene chloride (MC) and H₂O, and after removing the solvent, the result was purified by column chromatography using dichloromethane and hexane as a developing solvent to obtain Intermediate A1-3 (35 g, 77%).

### Preparation of Intermediate A1-2

In a one-neck round bottom flask, a mixture of Intermediate A1-3 (35 g, 145.07 mmol) and methylene chloride (MC) (500 ml) was cooled to 0° C., BBr₃ (72.69 g, 120.14 mmol) was added dropwise thereto, and, after raising the temperature to room temperature, the result was stirred for 1 hour. After the reaction was completed, the result was extracted with methylene chloride (MC) and H₂O, and after removing the solvent, the result was purified by column chromatography using dichloromethane and hexane as a developing solvent to obtain Intermediate A1-2 (20 g, 61%).

### Preparation of Intermediate A1-1

Intermediate A1-2 (20 g, 88.02 mmol), Cs₂CO₃ (71.69 g, 220.04 mmol) and dimethylacetamide (200 ml) were introduced into a one-neck round bottom flask, and stirred for 3 hours at 120° C. The result was cooled and then filtered, and after removing the solvent, the result was purified by column chromatography using dichloromethane and hexane as a developing solvent to obtain Intermediate A1-1 (17 g, 93%).

### Preparation of Intermediate A1

Intermediate A1-1 (17 g, 82.03 mmol), N-bromosuccinimide (NBS) (16.06 g, 90.24 mmol) and dimethylformamide (DMF) (200 ml) were introduced into a one-neck round bottom flask, and stirred for 3 hours at room temperature. After the reaction was completed, the result was extracted with methylene chloride (MC) and H₂O, and after removing the solvent, the result was purified by column chromatography using dichloromethane and hexane as a developing solvent to obtain Intermediate A1 (20 g, 85%).

### [Preparation Example 2] Preparation of Intermediate A2

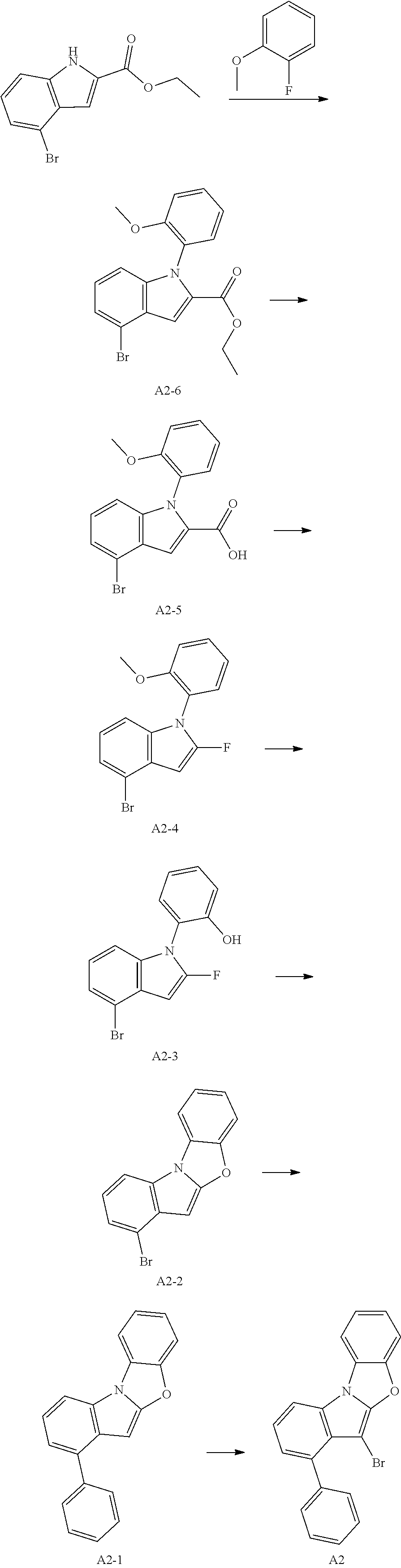

### Preparation of Intermediate A2-6

Intermediate A2-6 was synthesized in the same manner as in Preparation of A1-5 of Preparation Example 1 except that ethyl 4-bromo-1H-indole-2-carboxylate was used instead of ethyl 1H-indole-2-carboxylate.

### Preparation of Intermediate A2-5

Intermediate A2-5 was synthesized in the same manner as in Preparation of A1-4 of Preparation Example 1 except that Intermediate A2-6 was used instead of Intermediate A1-5.

### Preparation of Intermediate A2-4

Intermediate A2-4 was synthesized in the same manner as in Preparation of A1-3 of Preparation Example 1 except that Intermediate A2-5 was used instead of Intermediate A1-4.

### Preparation of Intermediate A2-3

Intermediate A2-3 was synthesized in the same manner as in Preparation of A1-2 of Preparation Example 1 except that Intermediate A2-4 was used instead of Intermediate A1-3.

### Preparation of Intermediate A2-2

Intermediate A2-2 was synthesized in the same manner as in Preparation of A1-1 of Preparation Example 1 except that Intermediate A2-3 was used instead of Intermediate A1-2.

### Preparation of Intermediate A2-1

Intermediate A2-2 (10 g, 34.95 mmol), phenylboronic acid (4.47 g, 36.70 mmol), K₂CO₃ (14.49 g, 104.85 mmol), Pd(PPh₃)₄ (1.21 g, 1.05 mmol), toluene (100 ml), EtOH (20 ml) and H₂O (20 ml) were introduced into a one-neck round bottom flask, and stirred for 12 hours under reflux. After the reaction was completed, the result was extracted with methylene chloride (MC) and H₂O, and after removing the solvent, the result was purified by column chromatography using dichloromethane and hexane as a developing solvent to obtain Intermediate A2-1 (8 g, 81%).

### Preparation of Intermediate A2

Intermediate A2 was synthesized in the same manner as in Preparation of A1 of Preparation Example 1 except that Intermediate A2-1 was used instead of Intermediate A1-1.

Intermediates were synthesized in the same manner as in Preparation Example 2 except that Si of the following Table 1 was used instead of ethyl 4-bromo-1H-indole-2-carboxylate, S2 of the following Table 1 was used instead of 1-fluoro-2-methoxybenzene, and S3 of the following Table 1 was used instead of phenylboronic acid.

TABLE 1

| Intermediate | S1 | S2 | S3 | Structure | Yield |
|---|---|---|---|---|---|
| A3 | 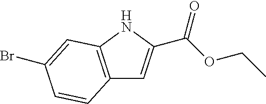 | 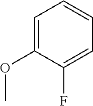 | 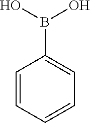 | 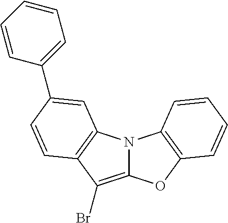 [SMILES: Brc1c2ccc(-c3ccccc3)cc2n2c1oc1ccccc12] | 82% |
| A4 | 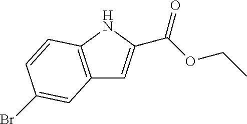 | 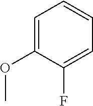 | 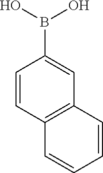 | 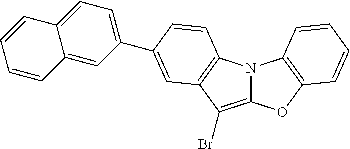 [SMILES: Brc1c2cc(-c3ccc4ccccc4c3)ccc2n2c1oc1ccccc12] | 77% |
| A5 | 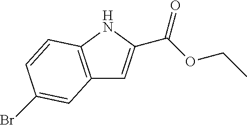 | 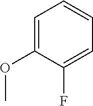 | 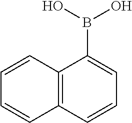 | 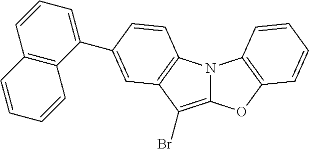 [SMILES: Brc1c2cc(-c3cccc4ccccc34)ccc2n2c1oc1ccccc12] | 83% |
| A6 | 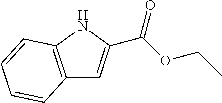 | 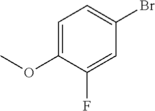 | 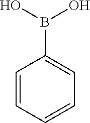 | 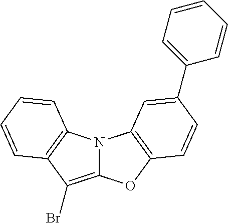 [SMILES: Brc1c2ccccc2n2c1oc1ccc(-c3ccccc3)cc12] | 80% |
| A7 | 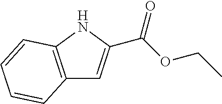 | 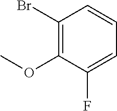 | 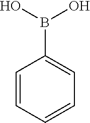 | 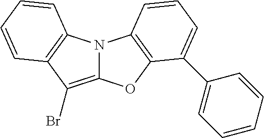 [SMILES: Brc1c2ccccc2n2c1oc1c(-c3ccccc3)cccc12] | 84% |
| A8 | 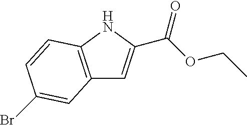 | 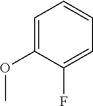 | 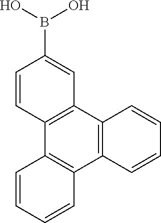 | 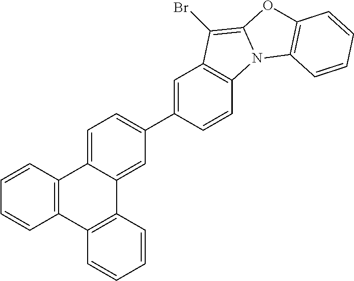 [SMILES: Brc1c2cc(-c3ccc4c5ccccc5c5ccccc5c4c3)ccc2n2c1oc1ccccc12] | 79% |
| A9 | 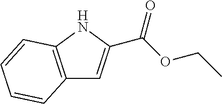 | 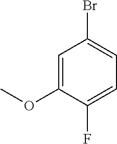 | 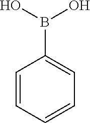 | 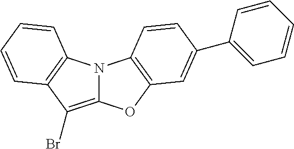 [SMILES: Brc1c2ccccc2n2c1oc1cc(-c3ccccc3)ccc12] | 73% |

### [Preparation Example 3] Preparation of Intermediate B1

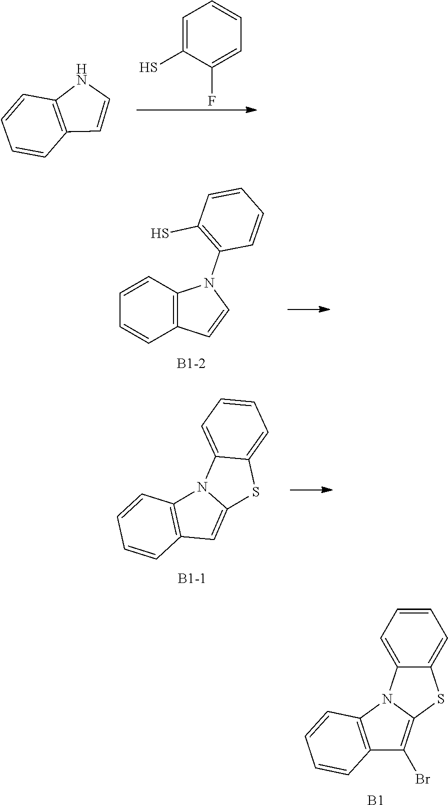

### Preparation of Intermediate B1-2

1H-indole (50 g, 426.80 mmol), 2-fluorobenzenethiol (60.17 g, 469.48 mmol), Cs₂CO₃ (417.18 g, 1280.41 mmol) and dimethylacetamide (500 ml) were introduced into a one-neck round bottom flask, and stirred for 12 hours under reflux. The result was cooled and then filtered, and after removing the solvent, the result was purified by column chromatography using dichloromethane and hexane as a developing solvent to obtain Intermediate B1-2 (85 g, 88%).

### Preparation of Intermediate B1-1

Intermediate B1-2 (85 g, 377.26 mmol), PdCl₂ (2.01 g, 11.32 mmol) and DMSO (850 ml) were introduced into a one-neck round bottom flask, and stirred for 12 hours at 140° C. After the reaction was completed, the result was extracted with methylene chloride (MC) and H₂O, and after removing the solvent, the result was purified by column chromatography using dichloromethane and hexane as a developing solvent to obtain Intermediate B1-1 (70 g, 83%).

### Preparation of Intermediate B1

Intermediate B1-1 (85 g, 380.67 mmol), N-bromosuccinimide (NBS) (74.53 g, 419.74 mmol) and dimethylformamide (DMF) (800 ml) were introduced into a one-neck round bottom flask, and stirred for 3 hours at room temperature. After the reaction was completed, the result was extracted with methylene chloride (MC) and H₂O, and after removing the solvent, the result was purified by column chromatography using dichloromethane and hexane as a developing solvent to obtain Intermediate B1 (90 g, 78%).

### [Preparation Example 4] Preparation of Intermediate B2

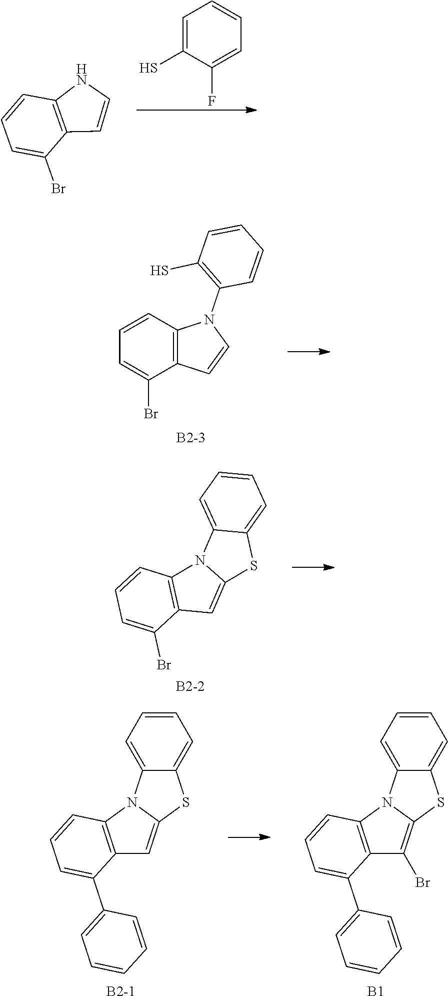

### Preparation of Intermediate B2-3

Intermediate B2-3 was synthesized in the same manner as in Preparation of B1-2 of Preparation Example 3 except that 4-bromo-1H-indole was used instead of 1H-indole.

### Preparation of Intermediate B2-2

Intermediate B2-2 was synthesized in the same manner as in Preparation of B1-1 of Preparation Example 3 except that Intermediate B2-3 was used instead of Intermediate B1-2.

### Preparation of Intermediate B2-1

Intermediate B2-1 was synthesized in the same manner as in Preparation of A2-1 of Preparation Example 2 except that Intermediate B2-2 was used instead of Intermediate A2-2.

### Preparation of Intermediate B2

Intermediate B2 was synthesized in the same manner as in Preparation of B1 of Preparation Example 3 except that Intermediate B2-1 was used instead of Intermediate B1-1.

Intermediates were synthesized in the same manner as in Preparation Example 4 except that S4 of the following Table 2 was used instead of 4-bromo-1H-indole, S5 of the following Table 2 was used instead of 2-fluorobenzenethiol, and S6 of the following Table 2 was used instead of phenylboronic acid.

TABLE 2

| Intermediate | S4 | S5 | S6 | Structure | Yield |
|---|---|---|---|---|---|
| B3 | 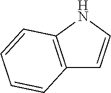 | 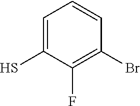 | 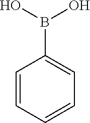 | 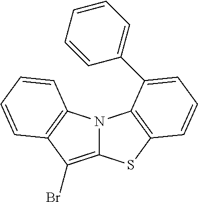 [SMILES: Brc1c2ccccc2n2c1sc1cccc(-c3ccccc3)c12] | 76% |
| B4 | 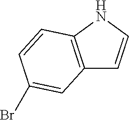 | 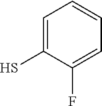 | 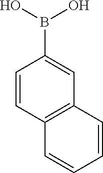 | 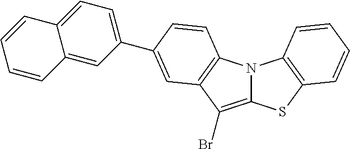 [SMILES: Brc1c2cc(-c3ccc4ccccc4c3)ccc2n2c1sc1ccccc12] | 80% |
| B5 | 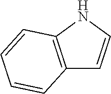 | 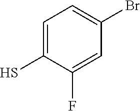 | 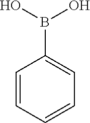 | 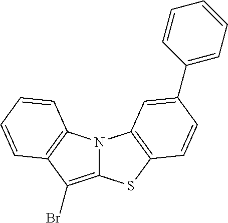 [SMILES: Brc1c2ccccc2n2c1sc1ccc(-c3ccccc3)cc12] | 79% |
| B6 | 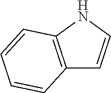 | 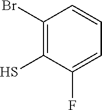 | 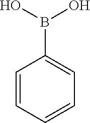 | 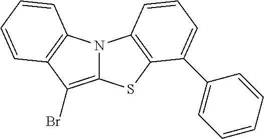 [SMILES: Brc1c2ccccc2n2c1sc1c(-c3ccccc3)cccc12] | 79% |
| B7 | 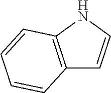 | 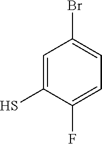 | 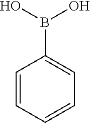 | 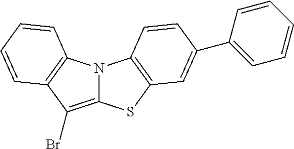 [SMILES: Brc1c2ccccc2n2c1sc1cc(-c3ccccc3)ccc12] | 82% |

### [Preparation Example 5] Preparation of Intermediate C1

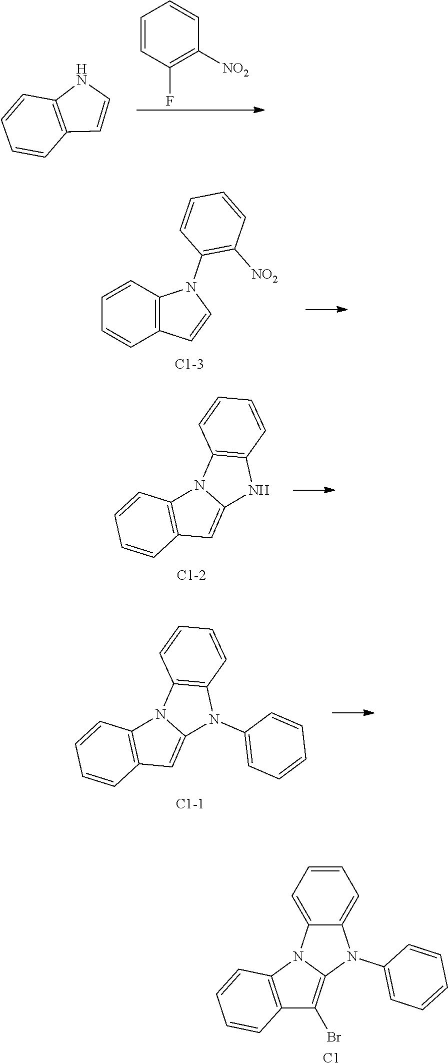

### Preparation of Intermediate C1-3

1H-indole (50 g, 426.80 mmol), 1-fluoro-2-nitrobenzene (66.24 g, 469.48 mmol), Cs₂CO₃ (417.18 g, 1280.41 mmol) and dimethylacetamide (500 ml) were introduced into a one-neck round bottom flask, and stirred for 12 hours under reflux. The result was cooled and then filtered, and after removing the solvent, the result was purified by column chromatography using dichloromethane and hexane as a developing solvent to obtain Intermediate C1-3 (85 g, 83%).

### Preparation of Intermediate C1-2

Intermediate C1-3 (85 g, 356.78 mmol), triphenylphosphine (233.95 g, 891.96 mmol) and 1,2-dichlorobenzene (900 ml) were introduced into a one-neck round bottom flask, and stirred for 12 hours under reflux. After the reaction was completed, the result was extracted with methylene chloride (MC) and H₂O, and after removing the solvent, the result was purified by column chromatography using dichloromethane and hexane as a developing solvent to obtain Intermediate C1-2 (65 g, 88%).

### Preparation of Intermediate C1-1

Intermediate C1-2 (65 g, 315.17 mmol), bromobenzene (51.96 g, 330.93 mmol), Pd(dba)₂ (9.06 g, 15.76 mmol), tri-tert-butylphosphine (50 wt %, 13 ml, 31.52 mmol), sodium tert-butoxide (75.72 g, 787.92 mmol) and toluene (700 ml) were introduced into a one-neck round bottom flask, and stirred for 12 hours under reflux. After the reaction was completed, the result was extracted with methylene chloride (MC) and H₂O, and after removing the solvent, the result was purified by column chromatography using dichloromethane and hexane as a developing solvent to obtain Intermediate C1-1 (76 g, 85%).

### Preparation of Intermediate C1

Intermediate C1-1 (76 g, 269.18 mmol), NBS (52.70 g, 296.10 mmol) and DMF (800 ml) were introduced into a one-neck round bottom flask, and stirred for 4 hours at room temperature. After the reaction was completed, the result was extracted with methylene chloride (MC) and H₂O, and after removing the solvent, the result was purified by column chromatography using dichloromethane and hexane as a developing solvent to obtain Intermediate C1 (84 g, 86%).

Intermediates were synthesized in the same manner as in Preparation Example 5 except that S7 of the following Table 3 was used instead of bromobenzene.

TABLE 3

| Intermediate | S7 | Structure | Yield |
|---|---|---|---|
| C2 | 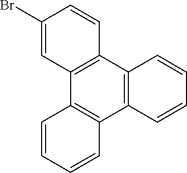 | 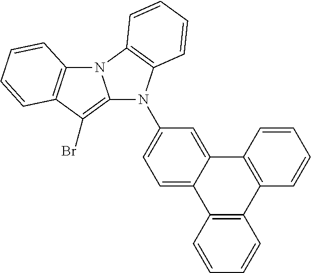 [SMILES: Brc1c2ccccc2n2c3ccccc3n(-c3ccc4c5ccccc5c5ccccc5c4c3)c12] | 76% |
| C3 | 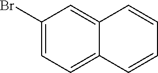 | 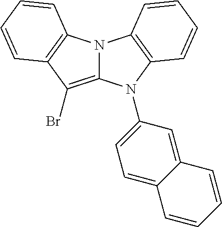 [SMILES: Brc1c2ccccc2n2c3ccccc3n(-c3ccc4ccccc4c3)c12] | 80% |
| C4 | 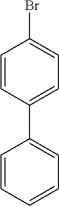 | 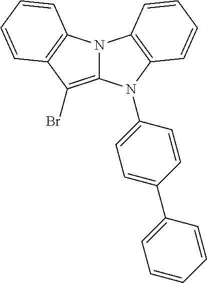 [SMILES: Brc1c2ccccc2n2c3ccccc3n(-c3ccc(-c4ccccc4)cc3)c12] | 79% |
| C5 | 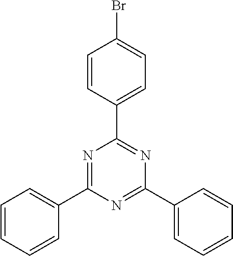 | 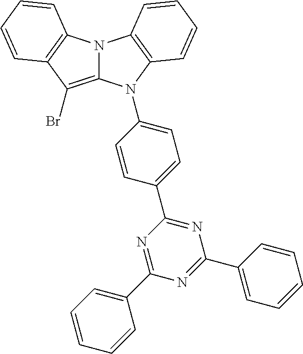 [SMILES: Brc1c2ccccc2n2c3ccccc3n(-c3ccc(-c4nc(-c5ccccc5)nc(-c5ccccc5)n4)cc3)c12] | 79% |

### [Preparation Example 6] Preparation of Compound 001

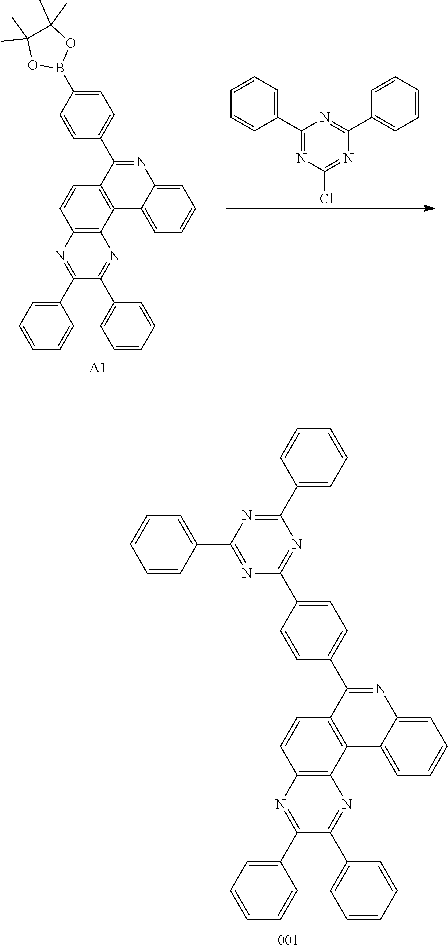

### Preparation of Compound 001

Intermediate A1 (8 g, 27.96 mmol), 2,4-diphenyl-6-(4-(4,4,5,5-tetramethyl-1,3,2-dioxaborolan-2-yl)phenyl)-1,3,5-triazine (12.78 g, 29.36 mmol), K₂CO₃ (11.59 g, 83.88 mmol), Pd(PPh₃)₄ (1.62 g, 1.40 mmol), toluene (100 ml), EtOH (20 ml) and H₂O (20 ml) were introduced into a one-neck round bottom flask, and stirred for 12 hours under reflux. After the reaction was completed, the result was extracted with methylene chloride (MC) and H₂O, and after removing the solvent, the result was purified by column chromatography using dichloromethane and hexane as a developing solvent to obtain Compound 001 (10 g, 69%).

Final compounds were synthesized in the same manner as in Preparation Example 6 except that Intermediate of the following Table 4 was used instead of Intermediate A1, and S8 of the following Table 4 was used instead of 2,4-diphenyl-6-(4-(4,4,5,5-tetramethyl-1,3,2-dioxaborolan-2-yl)phenyl)-1,3,5-triazine.

TABLE 4

| Compound | Intermediate | S8 | Structure | Yield |
|---|---|---|---|---|
| 003 | 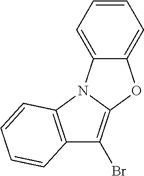 [SMILES: Brc1c2ccccc2n2c1oc1ccccc12] | 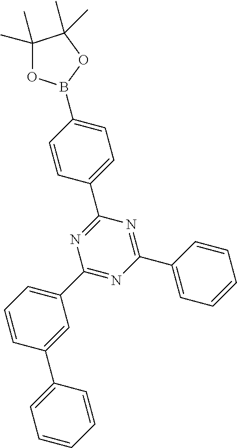 | 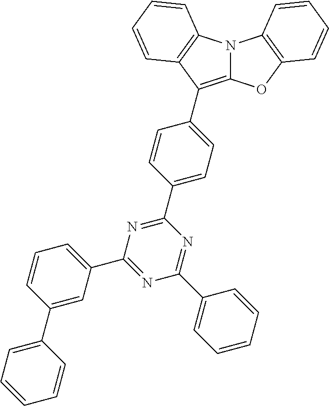 [SMILES: c1ccc(-c2cccc(-c3nc(-c4ccccc4)nc(-c4ccc(-c5c6ccccc6n6c5oc5ccccc56)cc4)n3)c2)cc1] | 76% |
| 005 | 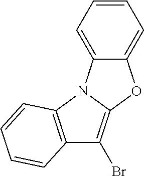 [SMILES: Brc1c2ccccc2n2c1oc1ccccc12] | 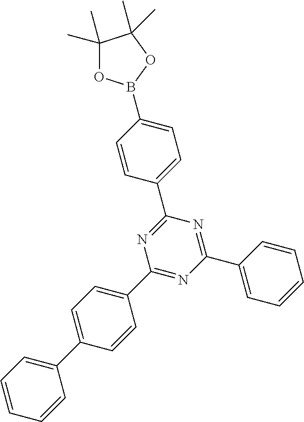 | 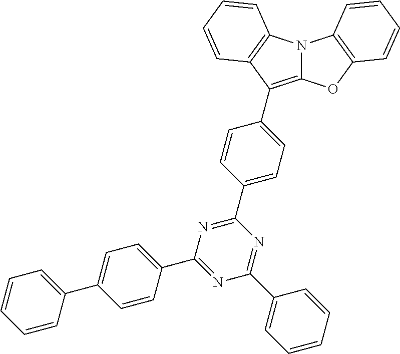 [SMILES: c1ccc(-c2ccc(-c3nc(-c4ccccc4)nc(-c4ccc(-c5c6ccccc6n6c5oc5ccccc56)cc4)n3)cc2)cc1] | 82% |
| 009 | 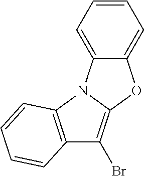 [SMILES: Brc1c2ccccc2n2c1oc1ccccc12] | 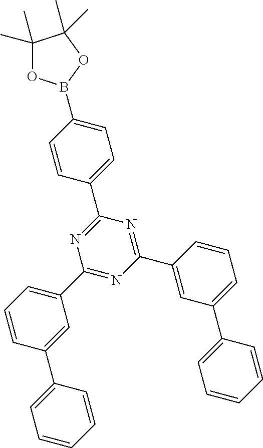 | 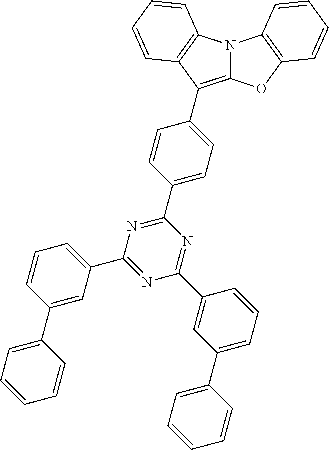 [SMILES: c1ccc(-c2cccc(-c3nc(-c4ccc(-c5c6ccccc6n6c5oc5ccccc56)cc4)nc(-c4cccc(-c5ccccc5)c4)n3)c2)cc1] | 79% |
| 011 | 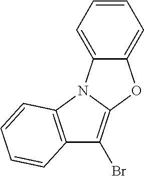 [SMILES: Brc1c2ccccc2n2c1oc1ccccc12] | 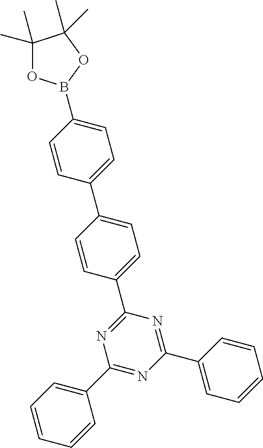 | 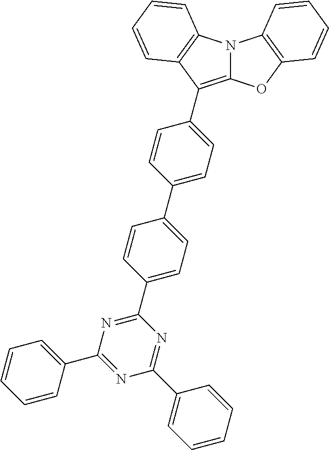 [SMILES: c1ccc(-c2nc(-c3ccccc3)nc(-c3ccc(-c4ccc(-c5c6ccccc6n6c5oc5ccccc56)cc4)cc3)n2)cc1] | 79% |
| 012 | 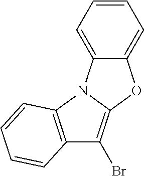 [SMILES: Brc1c2ccccc2n2c1oc1ccccc12] | 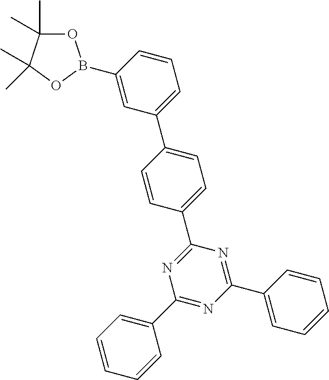 | 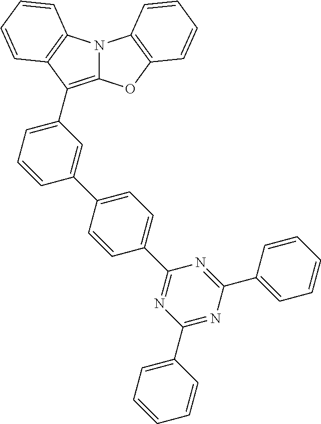 [SMILES: c1ccc(-c2nc(-c3ccccc3)nc(-c3ccc(-c4cccc(-c5c6ccccc6n6c5oc5ccccc56)c4)cc3)n2)cc1] | 82% |
| 014 | 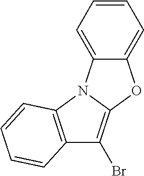 [SMILES: Brc1c2ccccc2n2c1oc1ccccc12] | 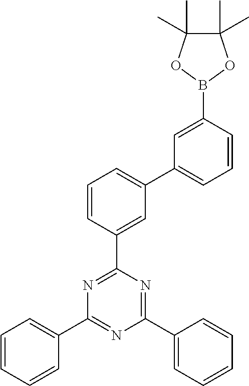 | 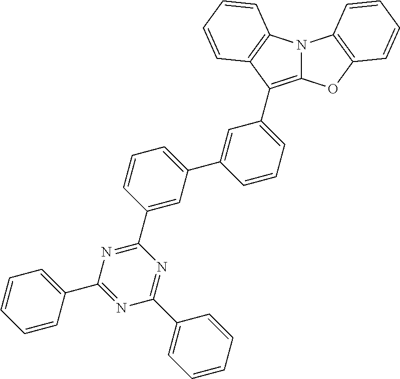 [SMILES: c1ccc(-c2nc(-c3ccccc3)nc(-c3cccc(-c4cccc(-c5c6ccccc6n6c5oc5ccccc56)c4)c3)n2)cc1] | 80% |
| 017 | 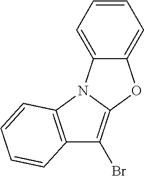 [SMILES: Brc1c2ccccc2n2c1oc1ccccc12] | 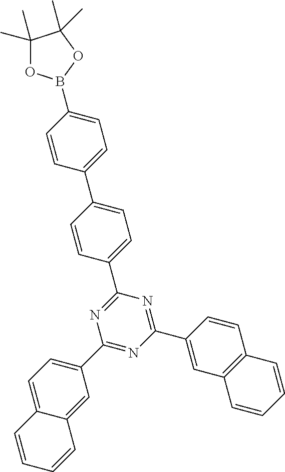 | 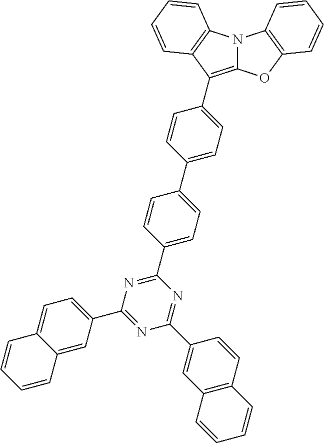 [SMILES: c1ccc2cc(-c3nc(-c4ccc(-c5ccc(-c6c7ccccc7n7c6oc6ccccc67)cc5)cc4)nc(-c4ccc5ccccc5c4)n3)ccc2c1] | 79% |
| 020 | 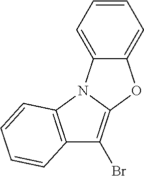 [SMILES: Brc1c2ccccc2n2c1oc1ccccc12] | 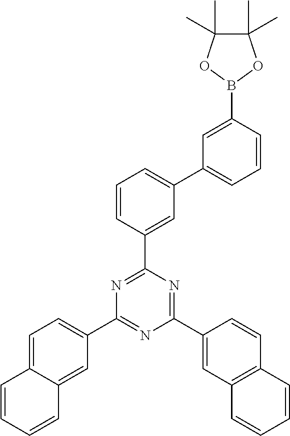 | 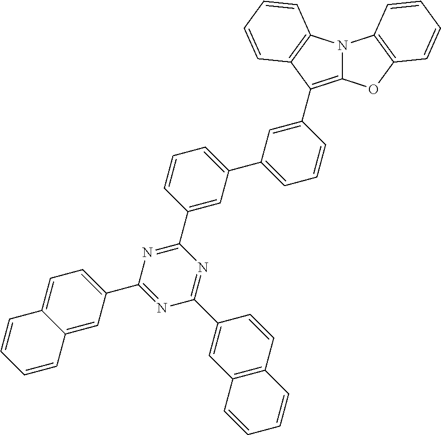 [SMILES: c1cc(-c2cccc(-c3c4ccccc4n4c3oc3ccccc34)c2)cc(-c2nc(-c3ccc4ccccc4c3)nc(-c3ccc4ccccc4c3)n2)c1] | 72% |
| 021 | 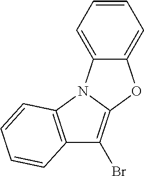 [SMILES: Brc1c2ccccc2n2c1oc1ccccc12] | 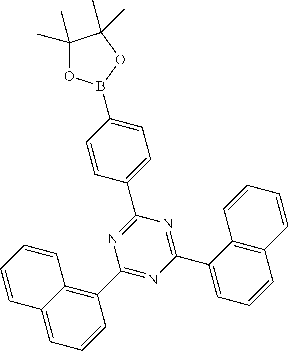 | 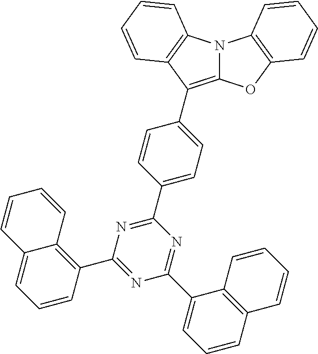 [SMILES: c1ccc2c(-c3nc(-c4ccc(-c5c6ccccc6n6c5oc5ccccc56)cc4)nc(-c4cccc5ccccc45)n3)cccc2c1] | 82% |
| 023 | 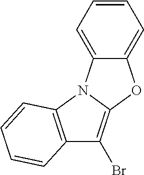 [SMILES: Brc1c2ccccc2n2c1oc1ccccc12] | 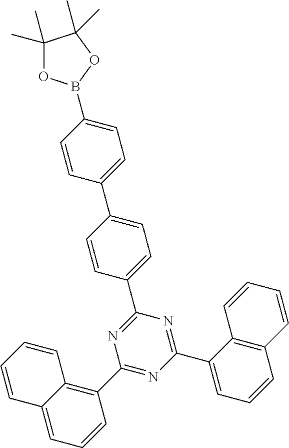 | 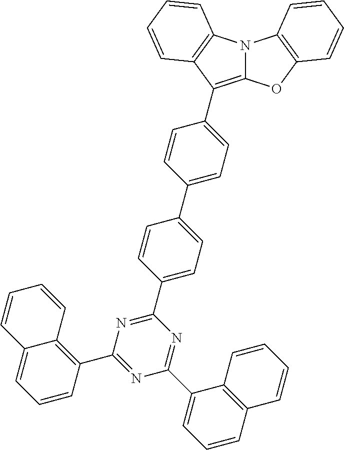 [SMILES: c1ccc2c(-c3nc(-c4ccc(-c5ccc(-c6c7ccccc7n7c6oc6ccccc67)cc5)cc4)nc(-c4cccc5ccccc45)n3)cccc2c1] | 80% |
| 030 | 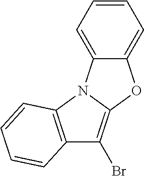 [SMILES: Brc1c2ccccc2n2c1oc1ccccc12] | 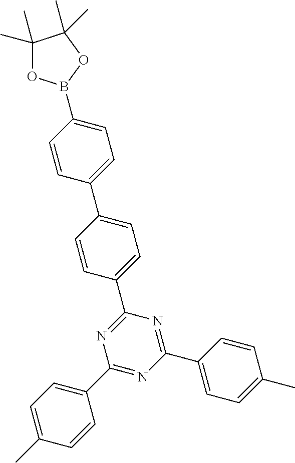 | 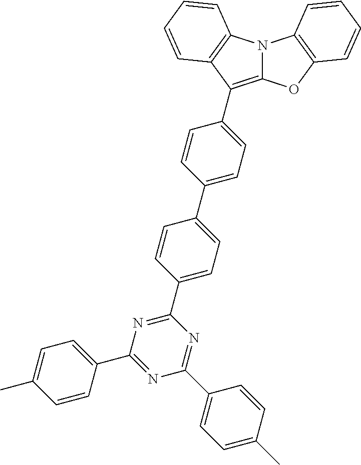 [SMILES: Cc1ccc(-c2nc(-c3ccc(C)cc3)nc(-c3ccc(-c4ccc(-c5c6ccccc6n6c5oc5ccccc56)cc4)cc3)n2)cc1] | 85% |
| 031 | 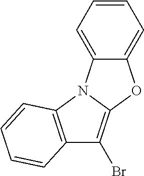 [SMILES: Brc1c2ccccc2n2c1oc1ccccc12] | 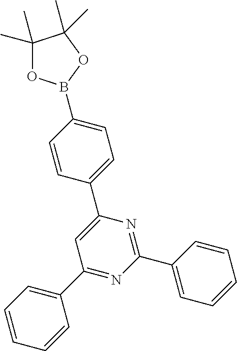 | 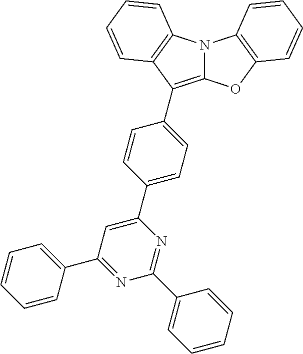 [SMILES: c1ccc(-c2cc(-c3ccc(-c4c5ccccc5n5c4oc4ccccc45)cc3)nc(-c3ccccc3)n2)cc1] | 79% |
| 033 | 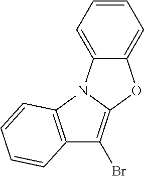 [SMILES: Brc1c2ccccc2n2c1oc1ccccc12] | 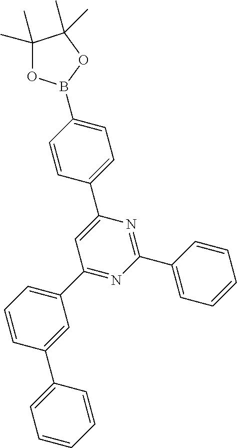 | 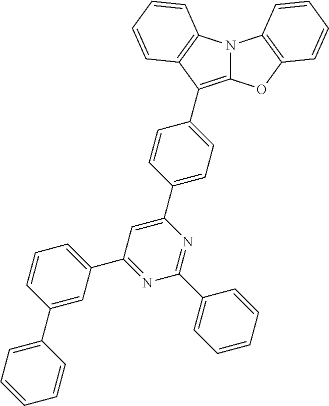 [SMILES: c1ccc(-c2cccc(-c3cc(-c4ccc(-c5c6ccccc6n6c5oc5ccccc56)cc4)nc(-c4ccccc4)n3)c2)cc1] | 72% |
| 035 | 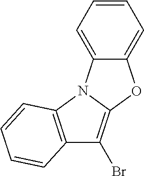 [SMILES: Brc1c2ccccc2n2c1oc1ccccc12] | 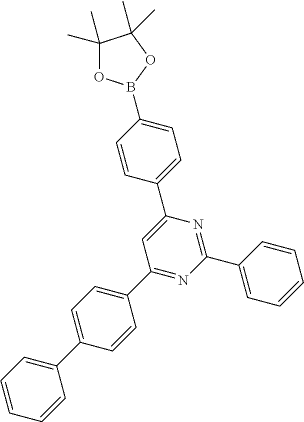 | 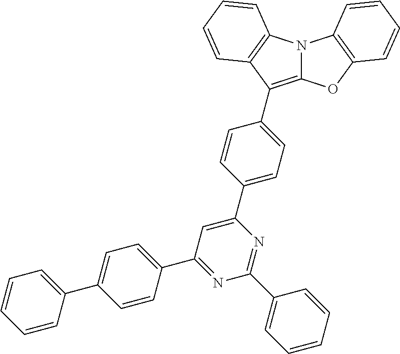 [SMILES: c1ccc(-c2ccc(-c3cc(-c4ccc(-c5c6ccccc6n6c5oc5ccccc56)cc4)nc(-c4ccccc4)n3)cc2)cc1] | 79% |
| 039 | 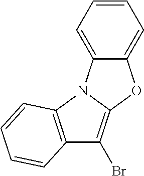 [SMILES: Brc1c2ccccc2n2c1oc1ccccc12] | 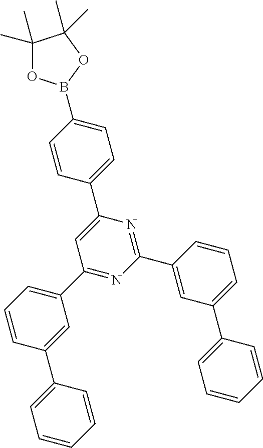 | 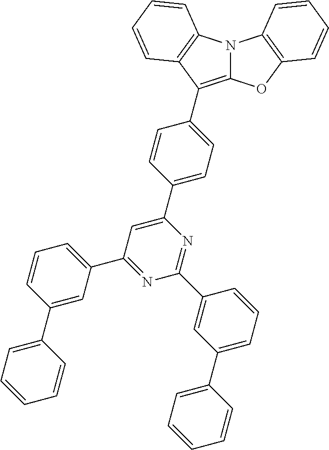 [SMILES: c1ccc(-c2cccc(-c3cc(-c4ccc(-c5c6ccccc6n6c5oc5ccccc56)cc4)nc(-c4cccc(-c5ccccc5)c4)n3)c2)cc1] | 82% |
| 041 | 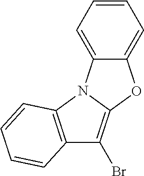 [SMILES: Brc1c2ccccc2n2c1oc1ccccc12] | 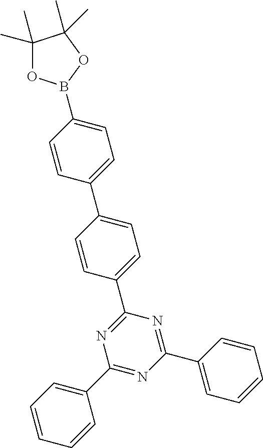 | 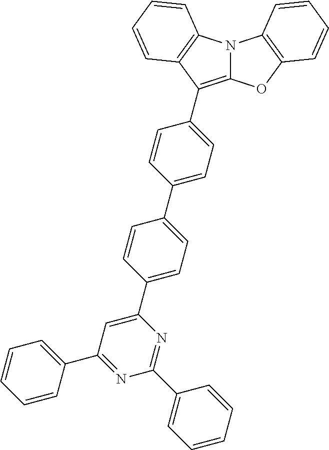 [SMILES: c1ccc(-c2cc(-c3ccc(-c4ccc(-c5c6ccccc6n6c5oc5ccccc56)cc4)cc3)nc(-c3ccccc3)n2)cc1] | 80% |
| 042 | 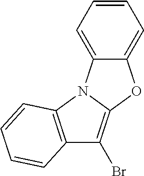 [SMILES: Brc1c2ccccc2n2c1oc1ccccc12] | 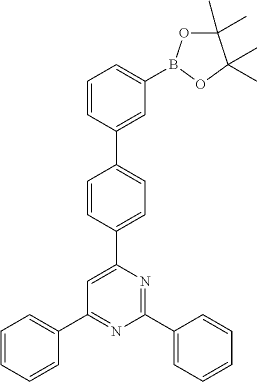 | 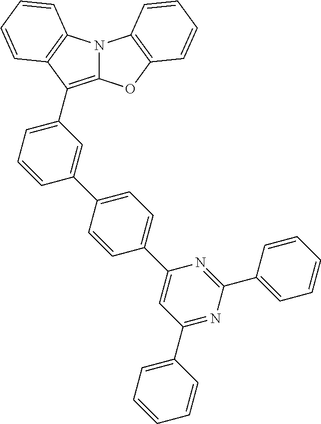 [SMILES: c1ccc(-c2cc(-c3ccc(-c4cccc(-c5c6ccccc6n6c5oc5ccccc56)c4)cc3)nc(-c3ccccc3)n2)cc1] | 79% |
| 043 | 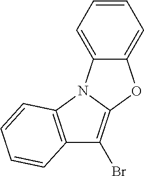 [SMILES: Brc1c2ccccc2n2c1oc1ccccc12] | 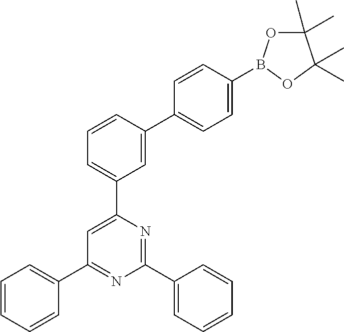 | 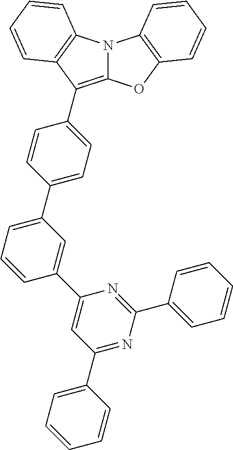 [SMILES: c1ccc(-c2cc(-c3cccc(-c4ccc(-c5c6ccccc6n6c5oc5ccccc56)cc4)c3)nc(-c3ccccc3)n2)cc1] | 71% |
| 044 | 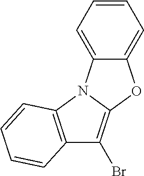 [SMILES: Brc1c2ccccc2n2c1oc1ccccc12] | 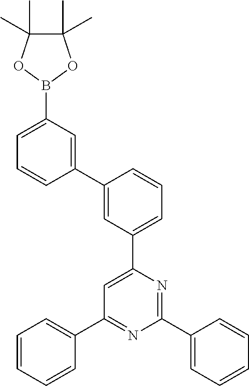 | 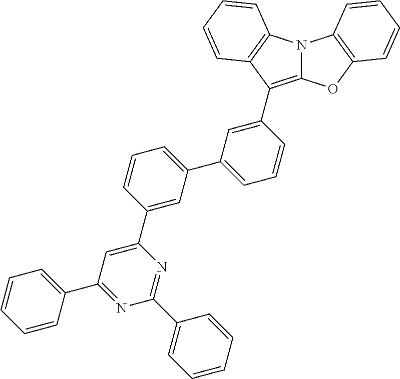 [SMILES: c1ccc(-c2cc(-c3cccc(-c4cccc(-c5c6ccccc6n6c5oc5ccccc56)c4)c3)nc(-c3ccccc3)n2)cc1] | 83% |
| 057 | 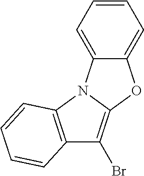 [SMILES: Brc1c2ccccc2n2c1oc1ccccc12] | 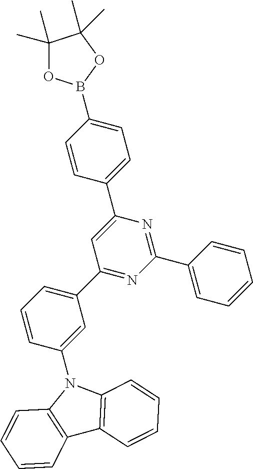 | 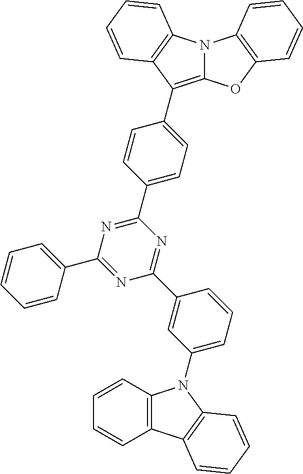 [SMILES: c1ccc(-c2nc(-c3ccc(-c4c5ccccc5n5c4oc4ccccc45)cc3)nc(-c3cccc(-n4c5ccccc5c5ccccc54)c3)n2)cc1] | 79% |
| 063 | 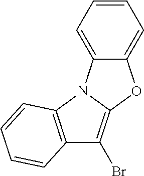 [SMILES: Brc1c2ccccc2n2c1oc1ccccc12] | 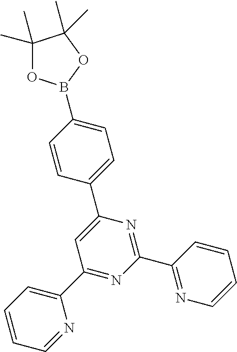 | 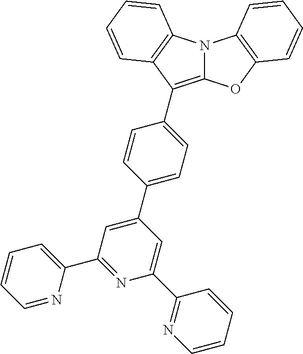 [SMILES: c1ccc(-c2cc(-c3ccc(-c4c5ccccc5n5c4oc4ccccc45)cc3)cc(-c3ccccn3)n2)nc1] | 82% |
| 065 | 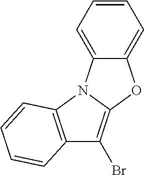 [SMILES: Brc1c2ccccc2n2c1oc1ccccc12] | 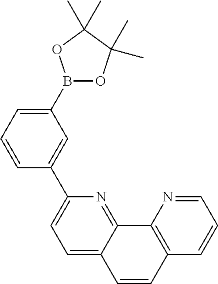 | 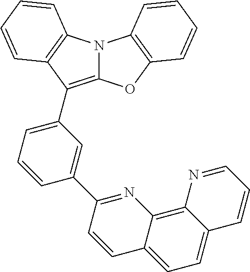 [SMILES: c1cc(-c2ccc3ccc4cccnc4c3n2)cc(-c2c3ccccc3n3c2oc2ccccc23)c1] | 80% |
| 067 | 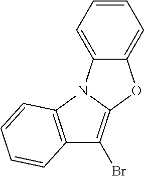 [SMILES: Brc1c2ccccc2n2c1oc1ccccc12] | 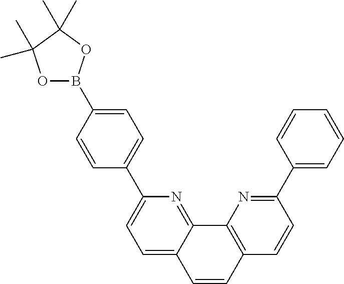 | 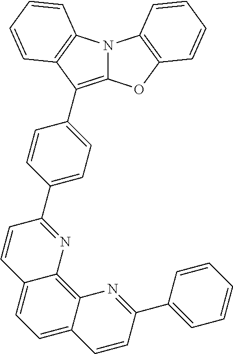 [SMILES: c1ccc(-c2ccc3ccc4ccc(-c5ccc(-c6c7ccccc7n7c6oc6ccccc67)cc5)nc4c3n2)cc1] | 79% |
| 068 | 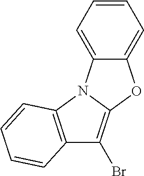 [SMILES: Brc1c2ccccc2n2c1oc1ccccc12] | 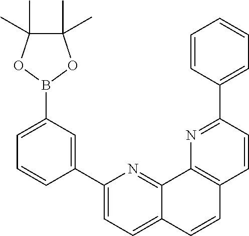 | 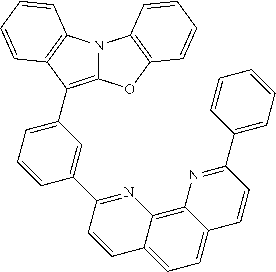 [SMILES: c1ccc(-c2ccc3ccc4ccc(-c5cccc(-c6c7ccccc7n7c6oc6ccccc67)c5)nc4c3n2)cc1] | 74% |
| 069 | 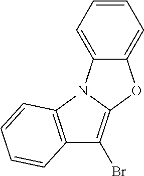 [SMILES: Brc1c2ccccc2n2c1oc1ccccc12] | 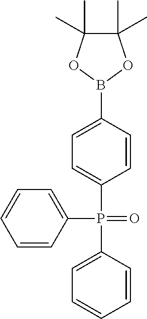 | 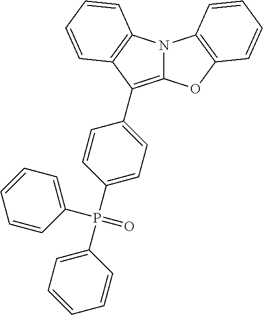 [SMILES: O=P(c1ccccc1)(c1ccccc1)c1ccc(-c2c3ccccc3n3c2oc2ccccc23)cc1] | 88% |
| 080 | 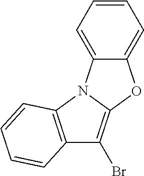 [SMILES: Brc1c2ccccc2n2c1oc1ccccc12] | 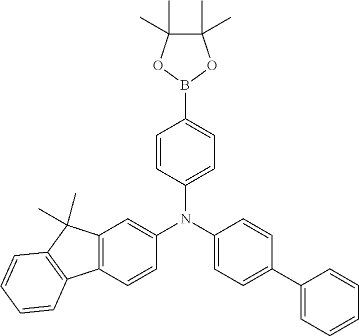 | 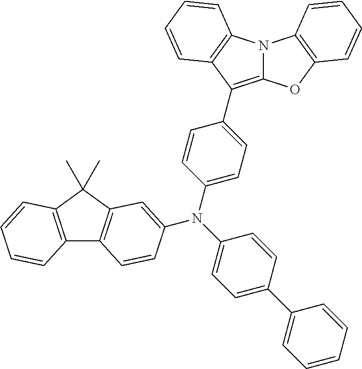 [SMILES: CC1(C)c2ccccc2-c2ccc(N(c3ccc(-c4ccccc4)cc3)c3ccc(-c4c5ccccc5n5c4oc4ccccc45)cc3)cc21] | 81% |
| 088 | 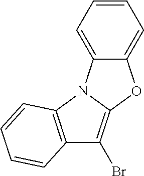 [SMILES: Brc1c2ccccc2n2c1oc1ccccc12] | 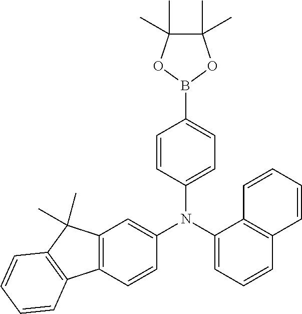 | 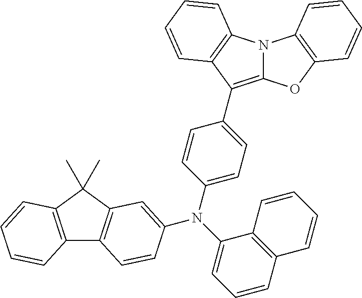 [SMILES: CC1(C)c2ccccc2-c2ccc(N(c3ccc(-c4c5ccccc5n5c4oc4ccccc45)cc3)c3cccc4ccccc34)cc21] | 79% |
| 092 | 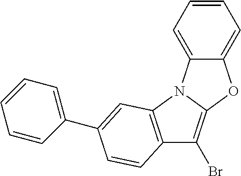 [SMILES: Brc1c2ccc(-c3ccccc3)cc2n2c1oc1ccccc12] | 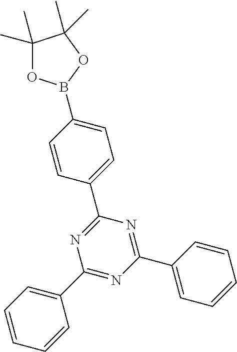 | 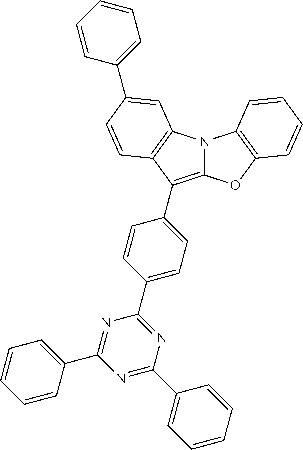 [SMILES: c1ccc(-c2ccc3c(-c4ccc(-c5nc(-c6ccccc6)nc(-c6ccccc6)n5)cc4)c4oc5ccccc5n4c3c2)cc1] | 79% |
| 101 | 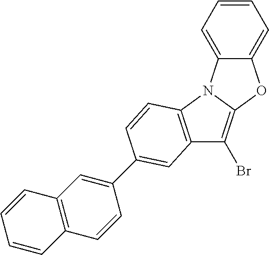 [SMILES: Brc1c2cc(-c3ccc4ccccc4c3)ccc2n2c1oc1ccccc12] | 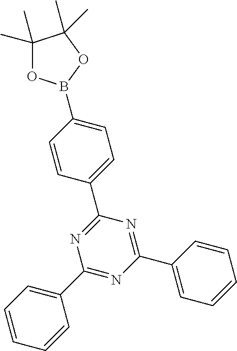 | 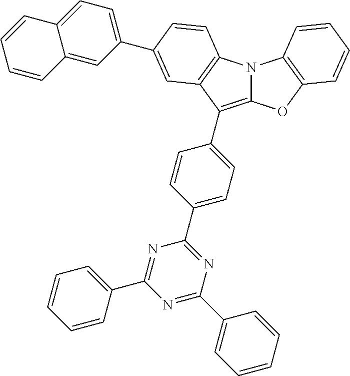 [SMILES: c1ccc(-c2nc(-c3ccccc3)nc(-c3ccc(-c4c5cc(-c6ccc7ccccc7c6)ccc5n5c4oc4ccccc45)cc3)n2)cc1] | 85% |
| 102 | 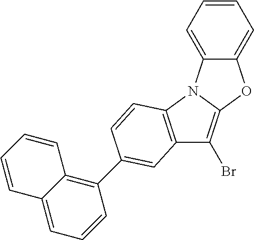 [SMILES: Brc1c2cc(-c3cccc4ccccc34)ccc2n2c1oc1ccccc12] | 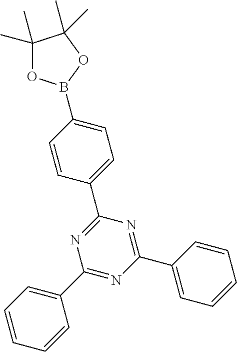 | 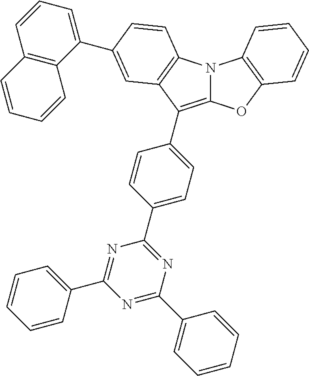 [SMILES: c1ccc(-c2nc(-c3ccccc3)nc(-c3ccc(-c4c5cc(-c6cccc7ccccc67)ccc5n5c4oc4ccccc45)cc3)n2)cc1] | 79% |
| 110 | 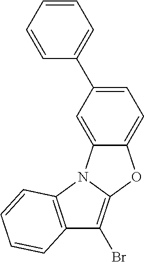 [SMILES: Brc1c2ccccc2n2c1oc1ccc(-c3ccccc3)cc12] | 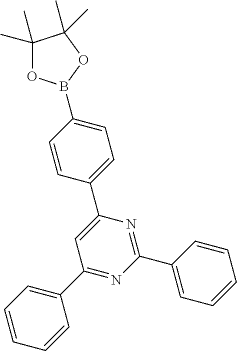 | 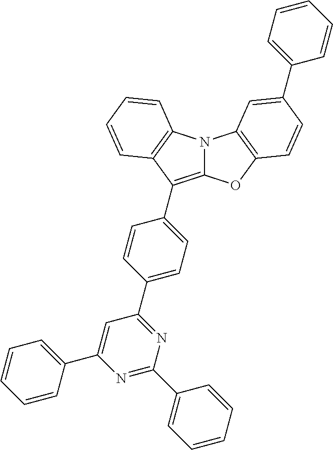 [SMILES: c1ccc(-c2ccc3oc4c(-c5ccc(-c6cc(-c7ccccc7)nc(-c7ccccc7)n6)cc5)c5ccccc5n4c3c2)cc1] | 72% |
| 112 | 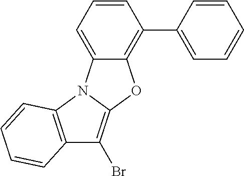 [SMILES: Brc1c2ccccc2n2c1oc1c(-c3ccccc3)cccc12] | 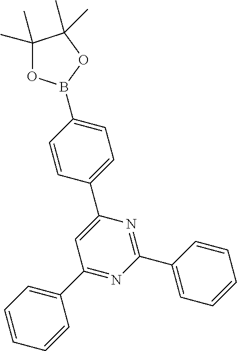 | 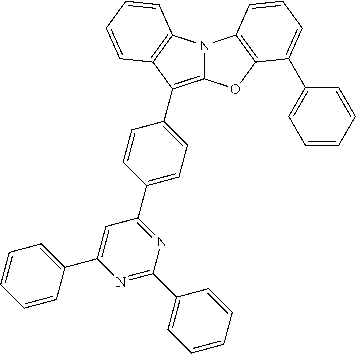 [SMILES: c1ccc(-c2cc(-c3ccc(-c4c5ccccc5n5c4oc4c(-c6ccccc6)cccc45)cc3)nc(-c3ccccc3)n2)cc1] | 79% |
| 119 | 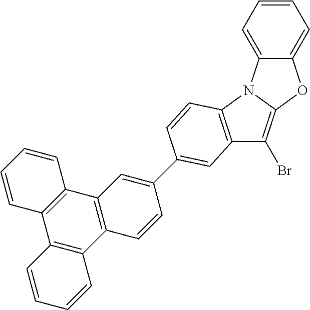 [SMILES: Brc1c2cc(-c3ccc4c5ccccc5c5ccccc5c4c3)ccc2n2c1oc1ccccc12] | 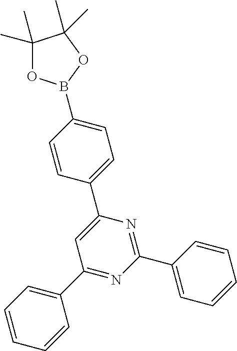 | 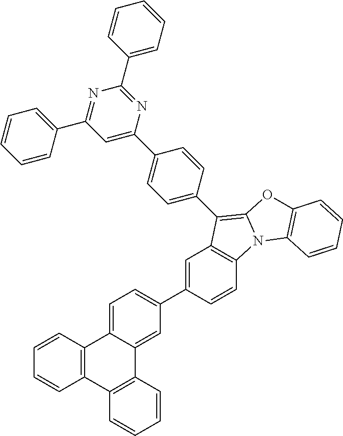 [SMILES: c1ccc(-c2cc(-c3ccc(-c4c5cc(-c6ccc7c8ccccc8c8ccccc8c7c6)ccc5n5c4oc4ccccc45)cc3)nc(-c3ccccc3)n2)cc1] | 82% |
| 126 | 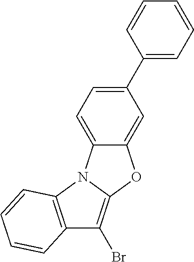 [SMILES: Brc1c2ccccc2n2c1oc1cc(-c3ccccc3)ccc12] | 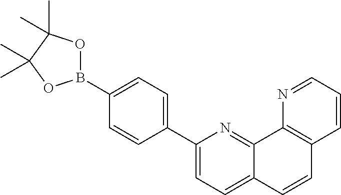 | 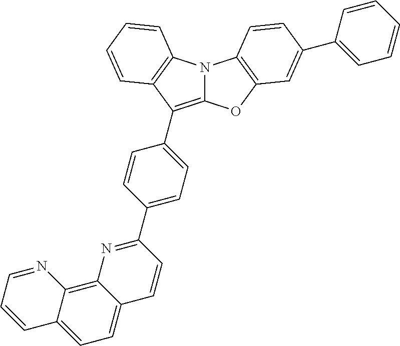 [SMILES: c1ccc(-c2ccc3c(c2)oc2c(-c4ccc(-c5ccc6ccc7cccnc7c6n5)cc4)c4ccccc4n23)cc1] | 80% |
| 127 | 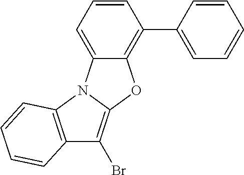 [SMILES: Brc1c2ccccc2n2c1oc1c(-c3ccccc3)cccc12] | 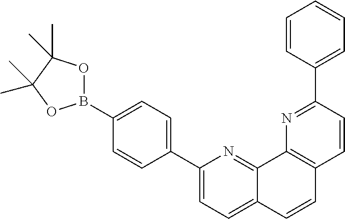 | 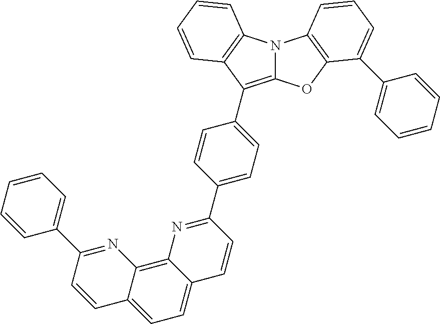 [SMILES: c1ccc(-c2ccc3ccc4ccc(-c5ccc(-c6c7ccccc7n7c6oc6c(-c8ccccc8)cccc67)cc5)nc4c3n2)cc1] | 79% |
| 132 | 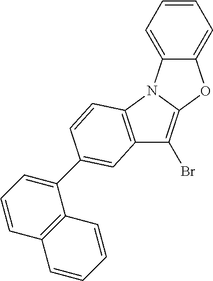 [SMILES: Brc1c2cc(-c3cccc4ccccc34)ccc2n2c1oc1ccccc12] | 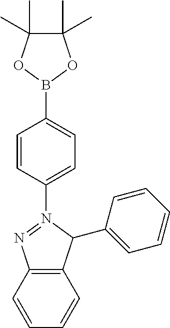 | 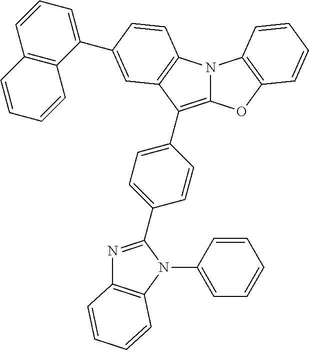 [SMILES: c1ccc(-n2c(-c3ccc(-c4c5cc(-c6cccc7ccccc67)ccc5n5c4oc4ccccc45)cc3)nc3ccccc32)cc1] | 83% |
| 140 | 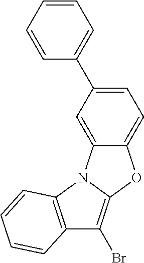 [SMILES: Brc1c2ccccc2n2c1oc1ccc(-c3ccccc3)cc12] | 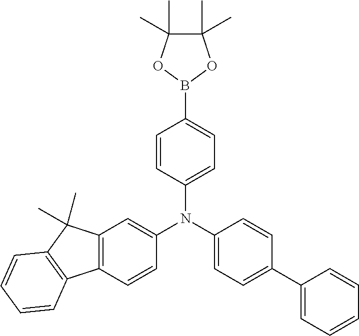 | 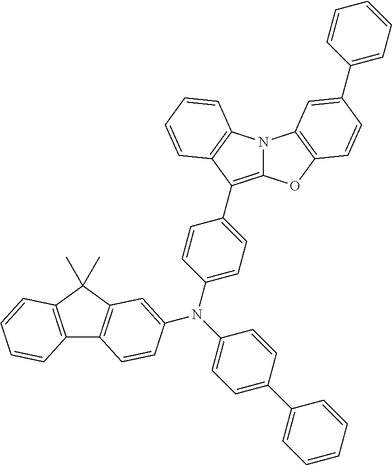 [SMILES: CC1(C)c2ccccc2-c2ccc(N(c3ccc(-c4ccccc4)cc3)c3ccc(-c4c5ccccc5n5c4oc4ccc(-c6ccccc6)cc45)cc3)cc21] | 84% |
| 141 | 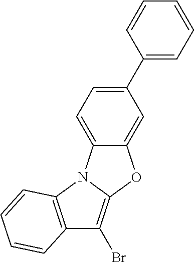 [SMILES: Brc1c2ccccc2n2c1oc1cc(-c3ccccc3)ccc12] | 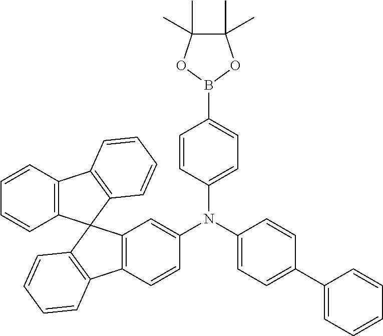 | 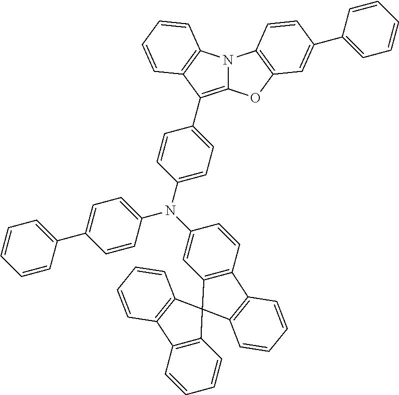 [SMILES: c1ccc(-c2ccc(N(c3ccc(-c4c5ccccc5n5c4oc4cc(-c6ccccc6)ccc45)cc3)c3ccc4c(c3)C3(c5ccccc5-c5ccccc53)c3ccccc3-4)cc2)cc1] | 79% |
| 151 | 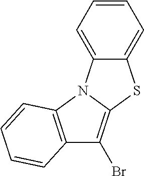 [SMILES: Brc1c2ccccc2n2c1sc1ccccc12] | 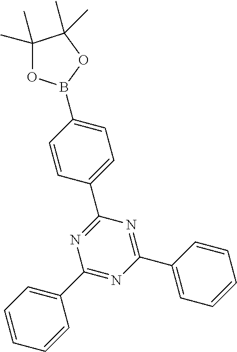 | 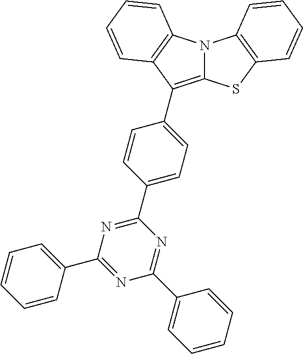 [SMILES: c1ccc(-c2nc(-c3ccccc3)nc(-c3ccc(-c4c5ccccc5n5c4sc4ccccc45)cc3)n2)cc1] | 79 |
| 152 | 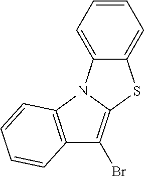 [SMILES: Brc1c2ccccc2n2c1sc1ccccc12] | 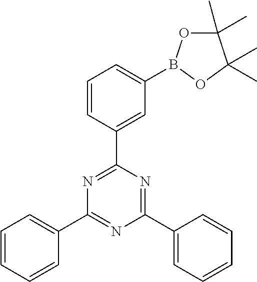 | 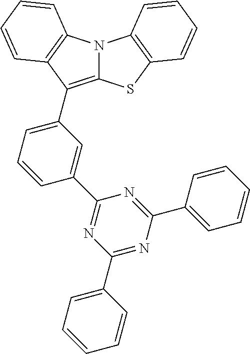 [SMILES: c1ccc(-c2nc(-c3ccccc3)nc(-c3cccc(-c4c5ccccc5n5c4sc4ccccc45)c3)n2)cc1] | 82% |
| 153 | 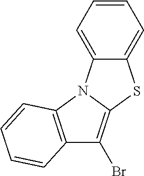 [SMILES: Brc1c2ccccc2n2c1sc1ccccc12] | 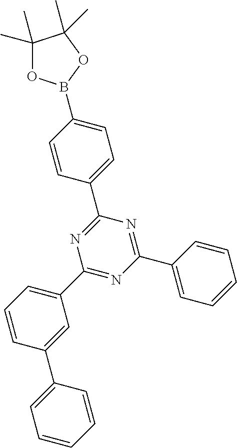 | 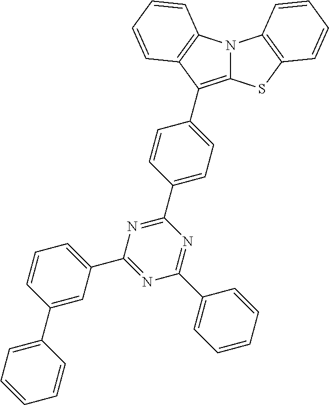 [SMILES: c1ccc(-c2cccc(-c3nc(-c4ccccc4)nc(-c4ccc(-c5c6ccccc6n6c5sc5ccccc56)cc4)n3)c2)cc1] | 80% |
| 161 | 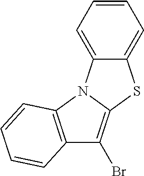 [SMILES: Brc1c2ccccc2n2c1sc1ccccc12] | 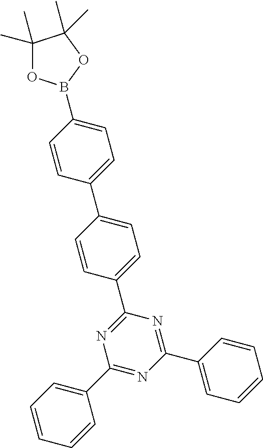 | 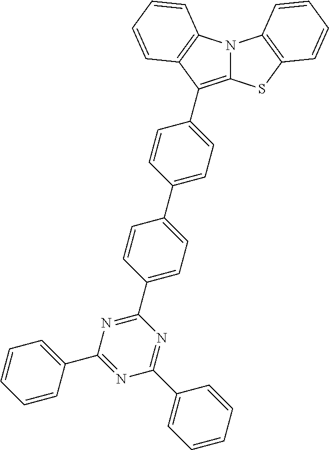 [SMILES: c1ccc(-c2nc(-c3ccccc3)nc(-c3ccc(-c4ccc(-c5c6ccccc6n6c5sc5ccccc56)cc4)cc3)n2)cc1] | 80% |
| 165 | 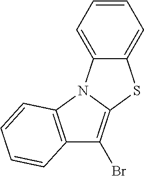 [SMILES: Brc1c2ccccc2n2c1sc1ccccc12] | 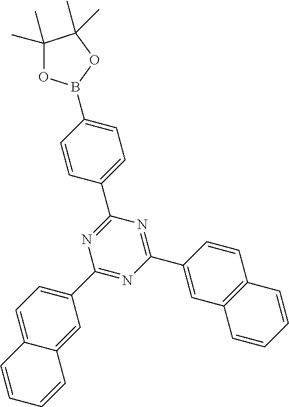 | 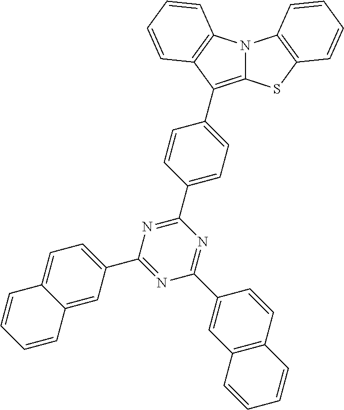 [SMILES: c1ccc2cc(-c3nc(-c4ccc(-c5c6ccccc6n6c5sc5ccccc56)cc4)nc(-c4ccc5ccccc5c4)n3)ccc2c1] | 79% |
| 173 | 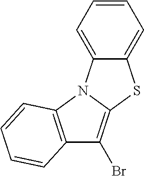 [SMILES: Brc1c2ccccc2n2c1sc1ccccc12] | 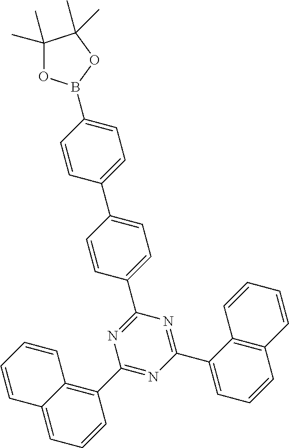 | 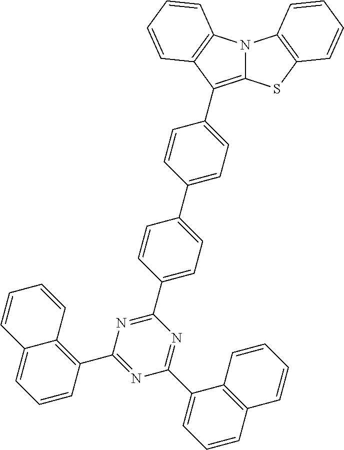 [SMILES: c1ccc2c(-c3nc(-c4ccc(-c5ccc(-c6c7ccccc7n7c6sc6ccccc67)cc5)cc4)nc(-c4cccc5ccccc45)n3)cccc2c1] | 71% |
| 177 | 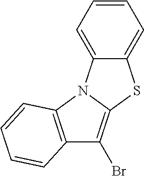 [SMILES: Brc1c2ccccc2n2c1sc1ccccc12] | 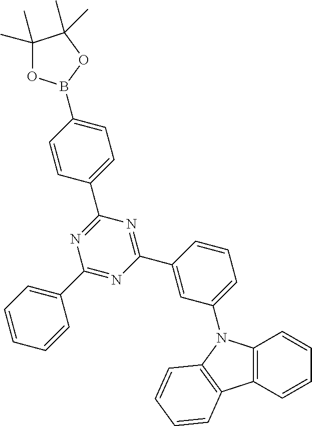 | 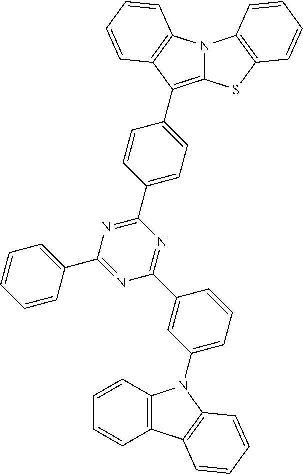 [SMILES: c1ccc(-c2nc(-c3ccc(-c4c5ccccc5n5c4sc4ccccc45)cc3)nc(-c3cccc(-n4c5ccccc5c5ccccc54)c3)n2)cc1] | 83% |
| 181 | 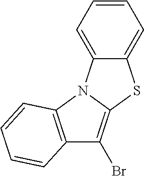 [SMILES: Brc1c2ccccc2n2c1sc1ccccc12] | 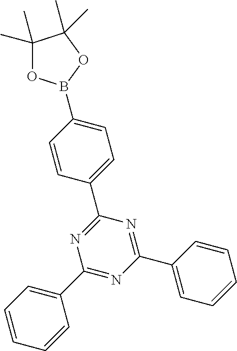 | 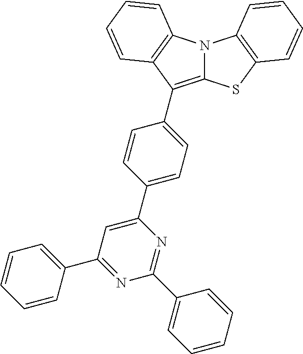 [SMILES: c1ccc(-c2cc(-c3ccc(-c4c5ccccc5n5c4sc4ccccc45)cc3)nc(-c3ccccc3)n2)cc1] | 82% |
| 187 | 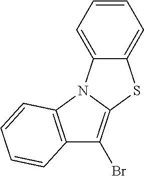 [SMILES: Brc1c2ccccc2n2c1sc1ccccc12] | 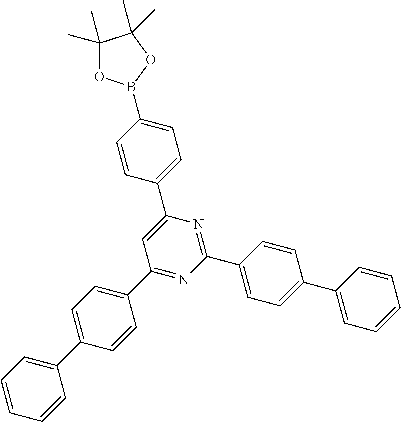 | 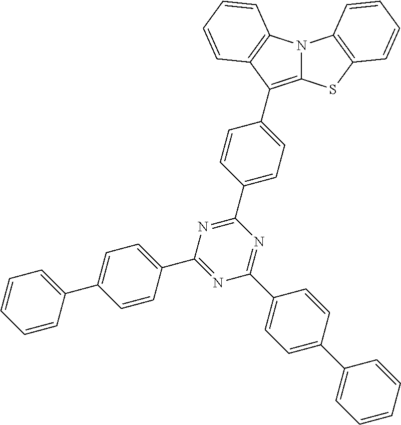 [SMILES: c1ccc(-c2ccc(-c3nc(-c4ccc(-c5ccccc5)cc4)nc(-c4ccc(-c5c6ccccc6n6c5sc5ccccc56)cc4)n3)cc2)cc1] | 79% |
| 191 | 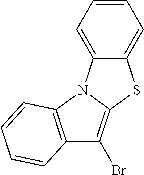 [SMILES: Brc1c2ccccc2n2c1sc1ccccc12] | 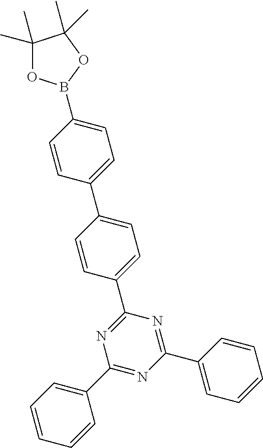 | 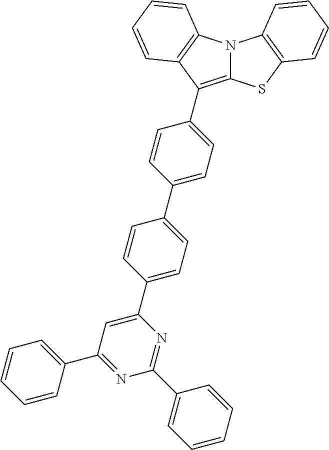 [SMILES: c1ccc(-c2cc(-c3ccc(-c4ccc(-c5c6ccccc6n6c5sc5ccccc56)cc4)cc3)nc(-c3ccccc3)n2)cc1] | 79% |
| 207 | 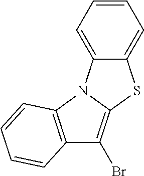 [SMILES: Brc1c2ccccc2n2c1sc1ccccc12] | 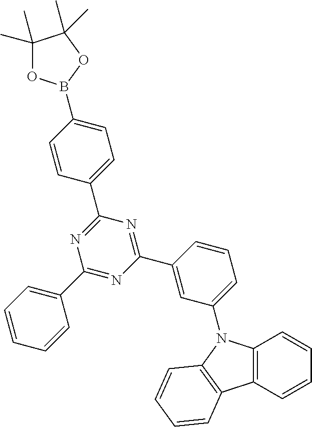 | 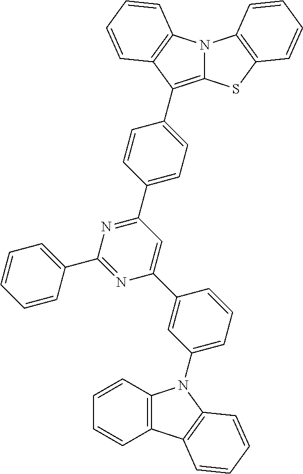 [SMILES: c1ccc(-c2nc(-c3ccc(-c4c5ccccc5n5c4sc4ccccc45)cc3)cc(-c3cccc(-n4c5ccccc5c5ccccc54)c3)n2)cc1] | 72% |
| 211 | 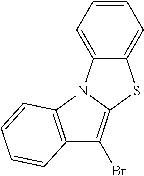 [SMILES: Brc1c2ccccc2n2c1sc1ccccc12] | 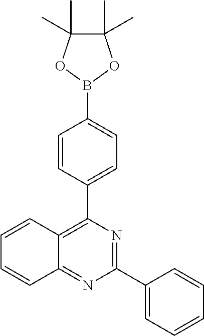 | 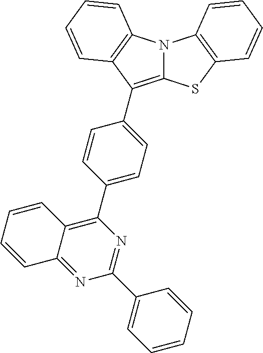 [SMILES: c1ccc(-c2nc(-c3ccc(-c4c5ccccc5n5c4sc4ccccc45)cc3)c3ccccc3n2)cc1] | 82% |
| 215 | 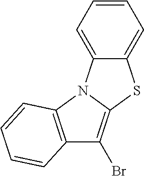 [SMILES: Brc1c2ccccc2n2c1sc1ccccc12] | 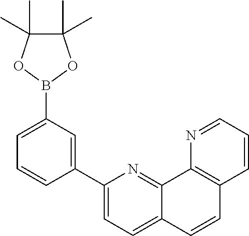 | 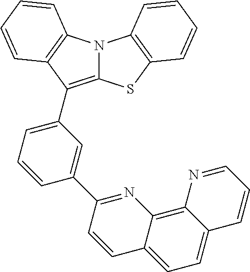 [SMILES: c1cc(-c2ccc3ccc4cccnc4c3n2)cc(-c2c3ccccc3n3c2sc2ccccc23)c1] | 80% |
| 217 | 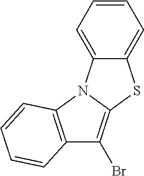 [SMILES: Brc1c2ccccc2n2c1sc1ccccc12] | 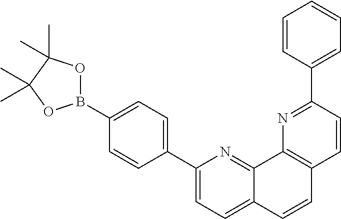 | 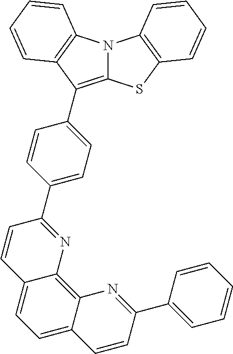 [SMILES: c1ccc(-c2ccc3ccc4ccc(-c5ccc(-c6c7ccccc7n7c6sc6ccccc67)cc5)nc4c3n2)cc1] | 85% |
| 218 | 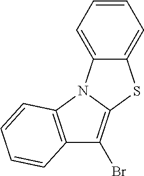 [SMILES: Brc1c2ccccc2n2c1sc1ccccc12] | 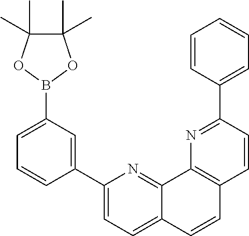 | 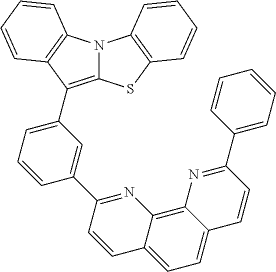 [SMILES: c1ccc(-c2ccc3ccc4ccc(-c5cccc(-c6c7ccccc7n7c6sc6ccccc67)c5)nc4c3n2)cc1] | 79% |
| 219 | 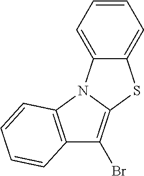 [SMILES: Brc1c2ccccc2n2c1sc1ccccc12] | 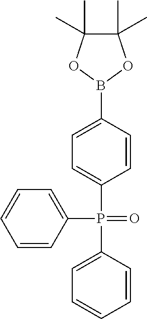 | 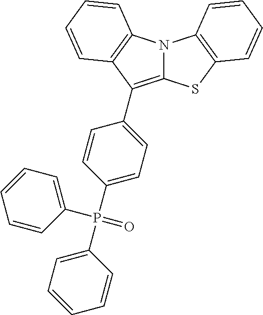 [SMILES: O=P(c1ccccc1)(c1ccccc1)c1ccc(-c2c3ccccc3n3c2sc2ccccc23)cc1] | 72% |
| 230 | 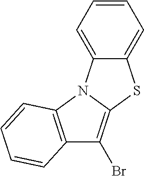 [SMILES: Brc1c2ccccc2n2c1sc1ccccc12] | 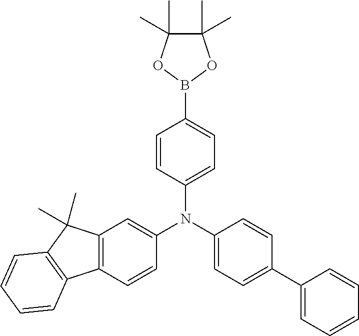 | 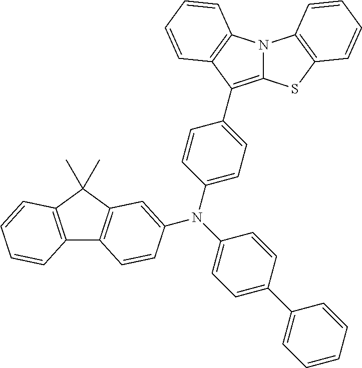 [SMILES: CC1(C)c2ccccc2-c2ccc(N(c3ccc(-c4ccccc4)cc3)c3ccc(-c4c5ccccc5n5c4sc4ccccc45)cc3)cc21] | 79% |
| 238 | 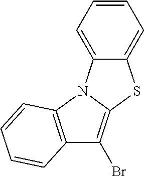 [SMILES: Brc1c2ccccc2n2c1sc1ccccc12] | 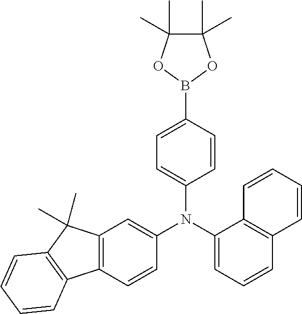 | 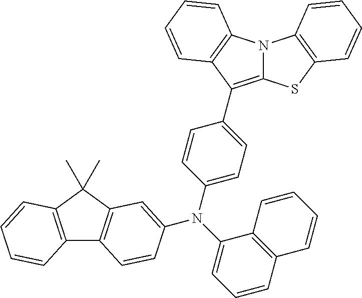 [SMILES: CC1(C)c2ccccc2-c2ccc(N(c3ccc(-c4c5ccccc5n5c4sc4ccccc45)cc3)c3cccc4ccccc34)cc21] | 82% |
| 243 | 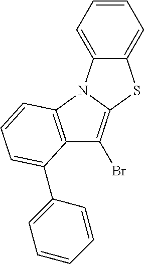 [SMILES: Brc1c2c(-c3ccccc3)cccc2n2c1sc1ccccc12] | 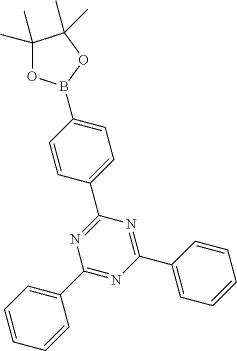 | 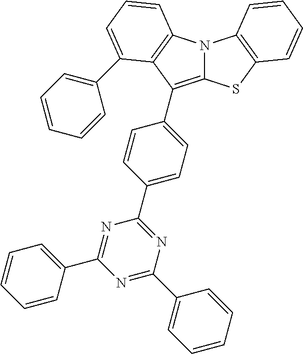 [SMILES: c1ccc(-c2nc(-c3ccccc3)nc(-c3ccc(-c4c5c(-c6ccccc6)cccc5n5c4sc4ccccc45)cc3)n2)cc1] | 80% |
| 244 | 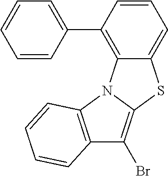 [SMILES: Brc1c2ccccc2n2c1sc1cccc(-c3ccccc3)c12] | 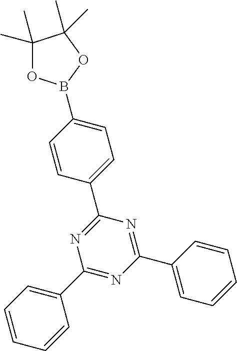 | 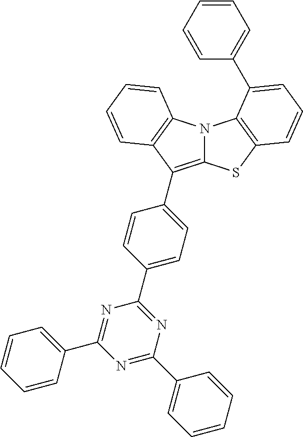 [SMILES: c1ccc(-c2nc(-c3ccccc3)nc(-c3ccc(-c4c5ccccc5n5c4sc4cccc(-c6ccccc6)c45)cc3)n2)cc1] | 79% |
| 251 | 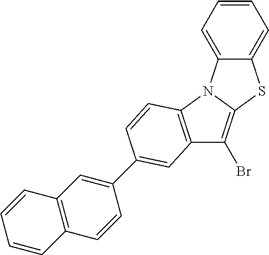 [SMILES: Brc1c2cc(-c3ccc4ccccc4c3)ccc2n2c1sc1ccccc12] | 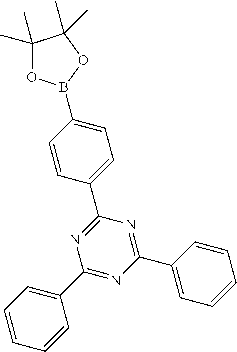 | 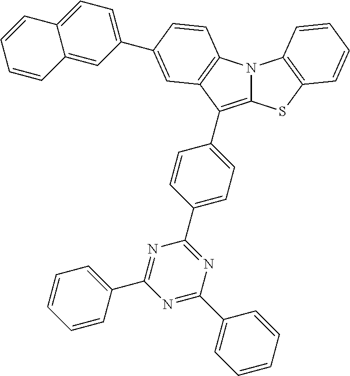 [SMILES: c1ccc(-c2nc(-c3ccccc3)nc(-c3ccc(-c4c5cc(-c6ccc7ccccc7c6)ccc5n5c4sc4ccccc45)cc3)n2)cc1] | 71% |
| 260 | 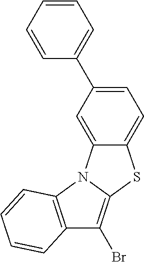 [SMILES: Brc1c2ccccc2n2c1sc1ccc(-c3ccccc3)cc12] | 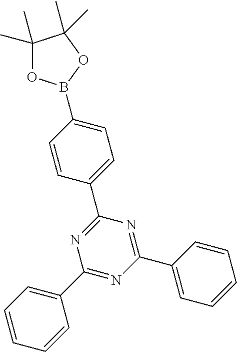 | 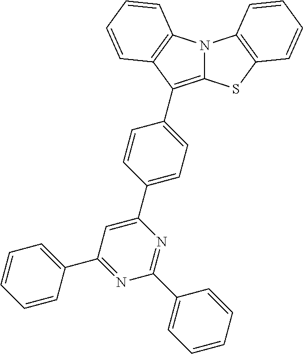 [SMILES: c1ccc(-c2cc(-c3ccc(-c4c5ccccc5n5c4sc4ccccc45)cc3)nc(-c3ccccc3)n2)cc1] | 80% |
| 262 | 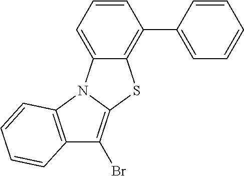 [SMILES: Brc1c2ccccc2n2c1sc1c(-c3ccccc3)cccc12] | 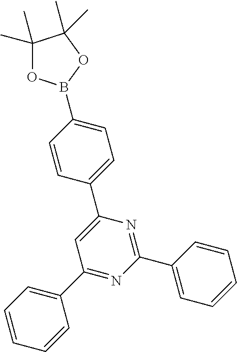 | 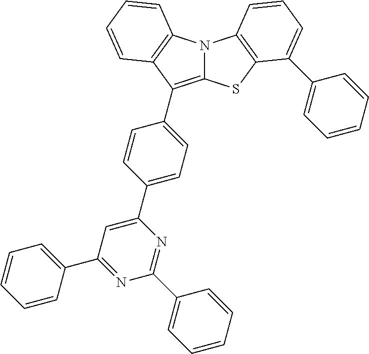 [SMILES: c1ccc(-c2cc(-c3ccc(-c4c5ccccc5n5c4sc4c(-c6ccccc6)cccc45)cc3)nc(-c3ccccc3)n2)cc1] | 79% |
| 277 | 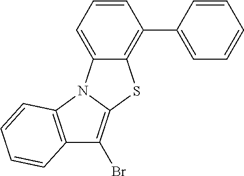 [SMILES: Brc1c2ccccc2n2c1sc1c(-c3ccccc3)cccc12] | 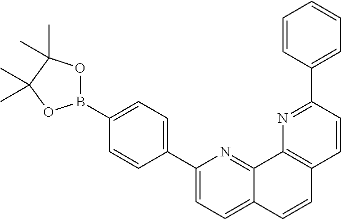 | 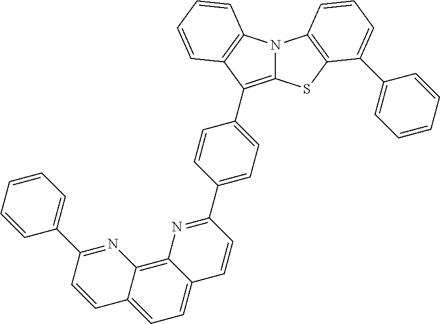 [SMILES: c1ccc(-c2ccc3ccc4ccc(-c5ccc(-c6c7ccccc7n7c6sc6c(-c8ccccc8)cccc67)cc5)nc4c3n2)cc1] | 71% |
| 281 | 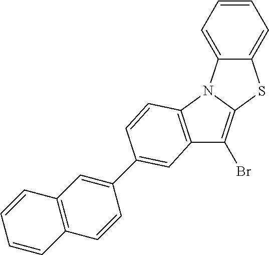 [SMILES: Brc1c2cc(-c3ccc4ccccc4c3)ccc2n2c1sc1ccccc12] | 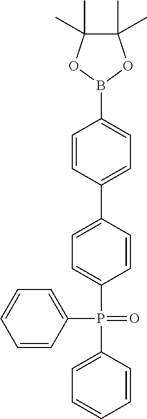 | 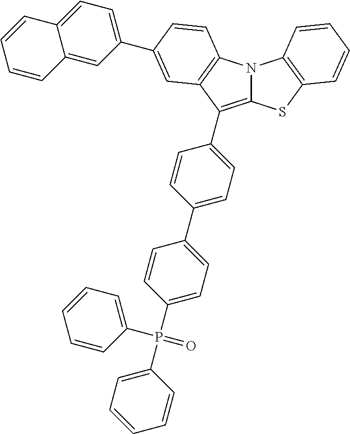 [SMILES: O=P(c1ccccc1)(c1ccccc1)c1ccc(-c2ccc(-c3c4cc(-c5ccc6ccccc6c5)ccc4n4c3sc3ccccc34)cc2)cc1] | 83% |
| 290 | 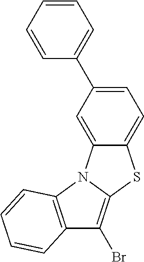 [SMILES: Brc1c2ccccc2n2c1sc1ccc(-c3ccccc3)cc12] | 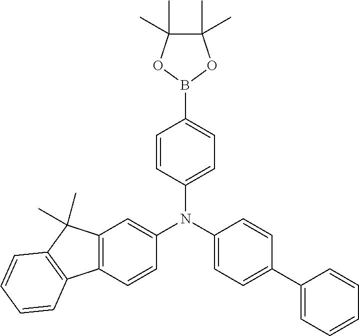 | 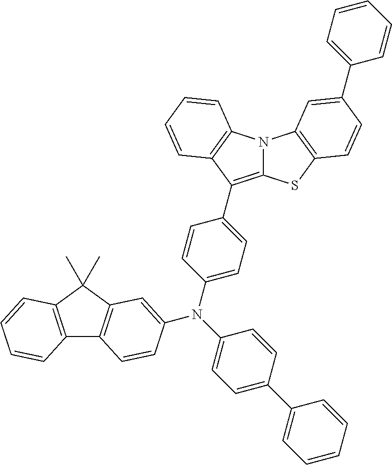 [SMILES: CC1(C)c2ccccc2-c2ccc(N(c3ccc(-c4ccccc4)cc3)c3ccc(-c4c5ccccc5n5c4sc4ccc(-c6ccccc6)cc45)cc3)cc21] | 82% |
| 291 | 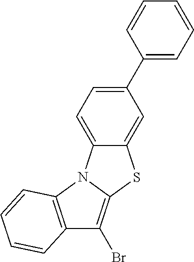 [SMILES: Brc1c2ccccc2n2c1sc1cc(-c3ccccc3)ccc12] | 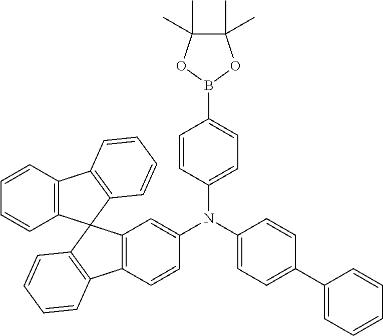 | 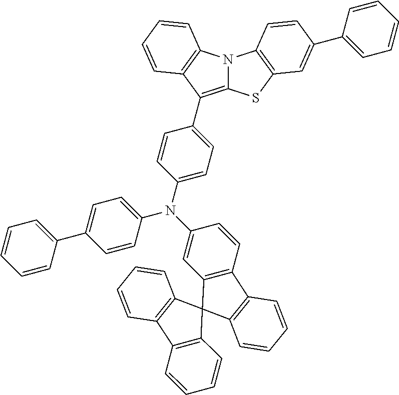 [SMILES: c1ccc(-c2ccc(N(c3ccc(-c4c5ccccc5n5c4sc4cc(-c6ccccc6)ccc45)cc3)c3ccc4c(c3)C3(c5ccccc5-c5ccccc53)c3ccccc3-4)cc2)cc1] | 790 |
| 301 | 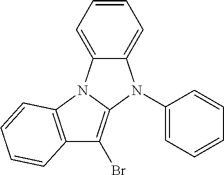 [SMILES: Brc1c2ccccc2n2c3ccccc3n(-c3ccccc3)c12] | 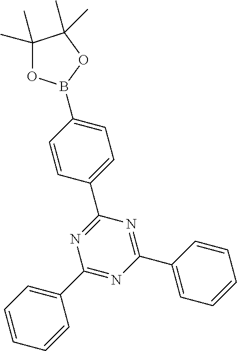 | 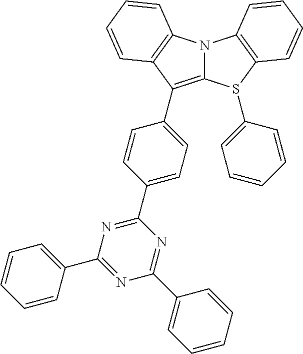 | 80% |
| 314 | 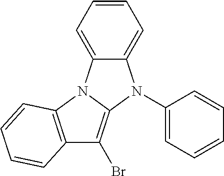 [SMILES: Brc1c2ccccc2n2c3ccccc3n(-c3ccccc3)c12] | 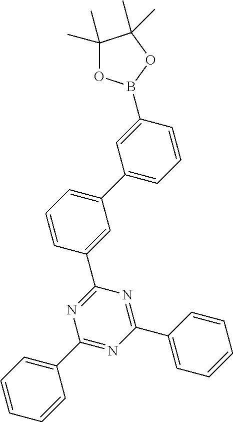 | 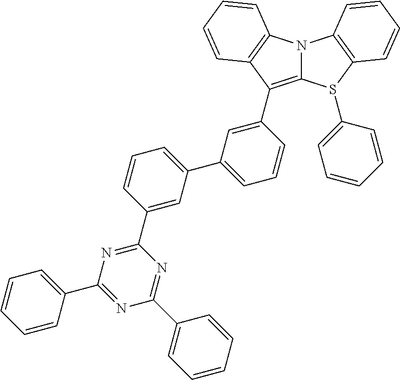 | 79% |
| 315 | 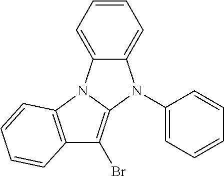 [SMILES: Brc1c2ccccc2n2c3ccccc3n(-c3ccccc3)c12] | 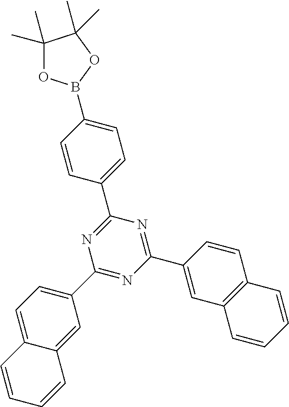 | 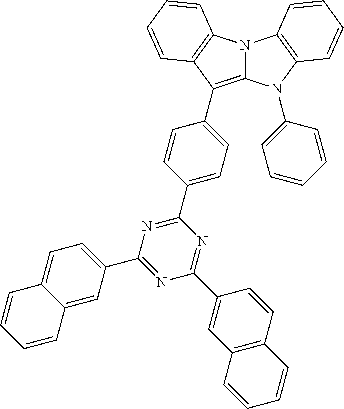 [SMILES: c1ccc(-n2c3ccccc3n3c4ccccc4c(-c4ccc(-c5nc(-c6ccc7ccccc7c6)nc(-c6ccc7ccccc7c6)n5)cc4)c23)cc1] | 72% |
| 329 | 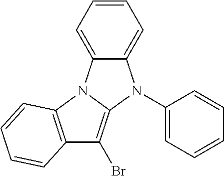 [SMILES: Brc1c2ccccc2n2c3ccccc3n(-c3ccccc3)c12] | 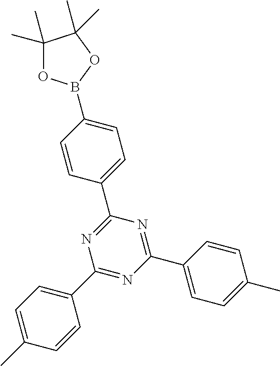 | 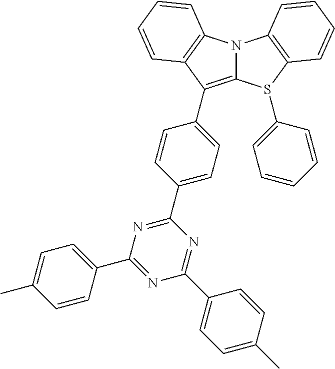 | 82% |
| 331 | 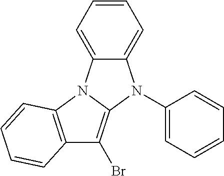 [SMILES: Brc1c2ccccc2n2c3ccccc3n(-c3ccccc3)c12] | 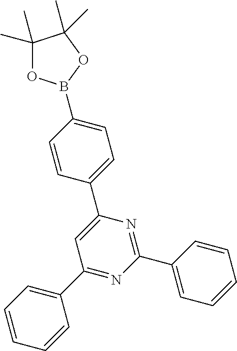 | 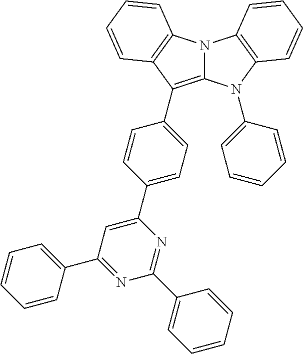 [SMILES: c1ccc(-c2cc(-c3ccc(-c4c5ccccc5n5c6ccccc6n(-c6ccccc6)c45)cc3)nc(-c3ccccc3)n2)cc1] | 80% |
| 341 | 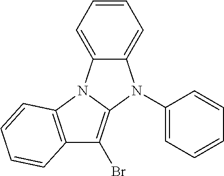 [SMILES: Brc1c2ccccc2n2c3ccccc3n(-c3ccccc3)c12] | 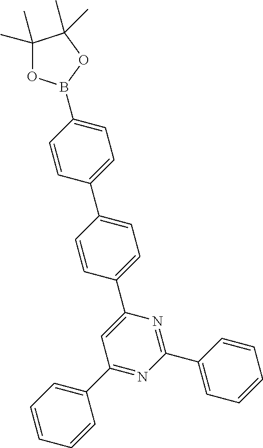 | 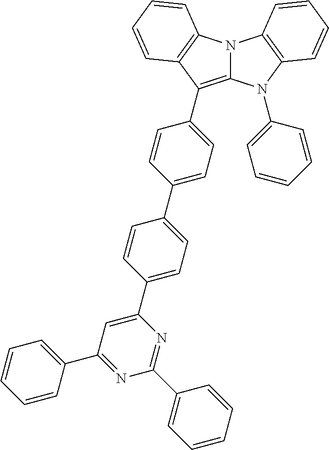 [SMILES: c1ccc(-c2cc(-c3ccc(-c4ccc(-c5c6ccccc6n6c7ccccc7n(-c7ccccc7)c56)cc4)cc3)nc(-c3ccccc3)n2)cc1] | 85% |
| 342 | 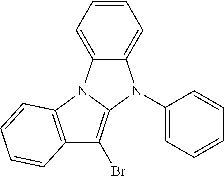 [SMILES: Brc1c2ccccc2n2c3ccccc3n(-c3ccccc3)c12] | 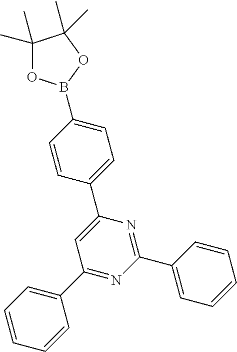 | 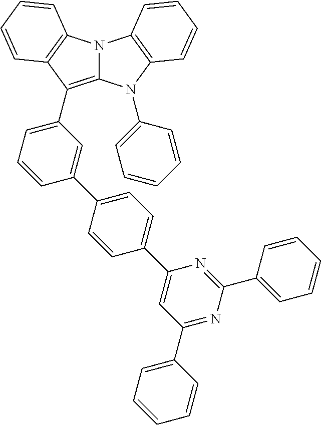 [SMILES: c1ccc(-c2cc(-c3ccc(-c4cccc(-c5c6ccccc6n6c7ccccc7n(-c7ccccc7)c56)c4)cc3)nc(-c3ccccc3)n2)cc1] | 79% |
| 357 | 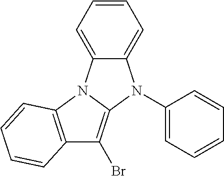 [SMILES: Brc1c2ccccc2n2c3ccccc3n(-c3ccccc3)c12] | 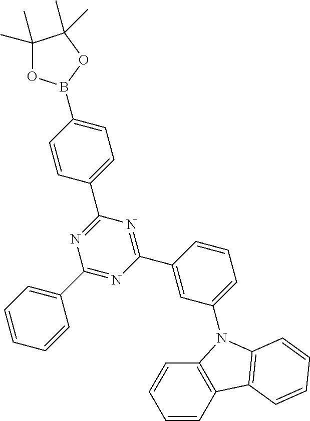 | 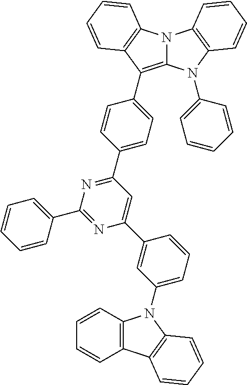 [SMILES: c1ccc(-c2nc(-c3ccc(-c4c5ccccc5n5c6ccccc6n(-c6ccccc6)c45)cc3)cc(-c3cccc(-n4c5ccccc5c5ccccc54)c3)n2)cc1] | 72% |
| 361 | 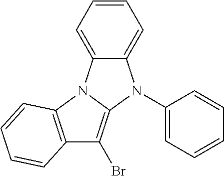 [SMILES: Brc1c2ccccc2n2c3ccccc3n(-c3ccccc3)c12] | 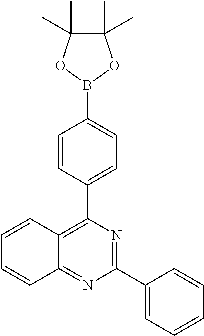 | 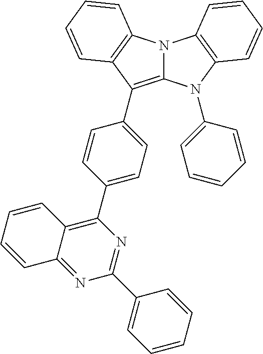 [SMILES: c1ccc(-c2nc(-c3ccc(-c4c5ccccc5n5c6ccccc6n(-c6ccccc6)c45)cc3)c3ccccc3n2)cc1] | 79% |
| 373 | 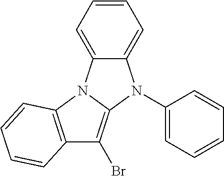 [SMILES: Brc1c2ccccc2n2c3ccccc3n(-c3ccccc3)c12] | 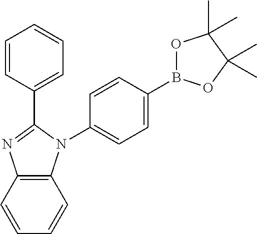 | 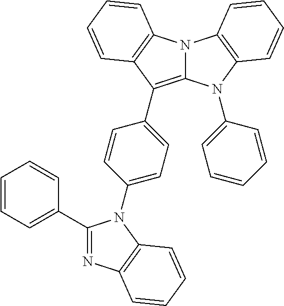 [SMILES: c1ccc(-c2nc3ccccc3n2-c2ccc(-c3c4ccccc4n4c5ccccc5n(-c5ccccc5)c34)cc2)cc1] | 82% |
| 380 | 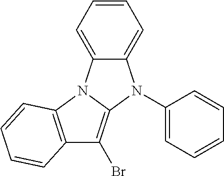 [SMILES: Brc1c2ccccc2n2c3ccccc3n(-c3ccccc3)c12] | 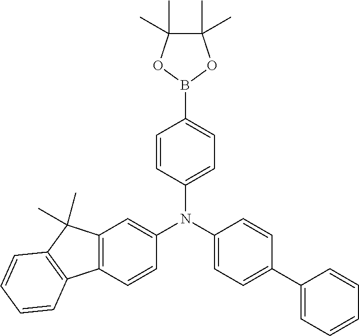 | 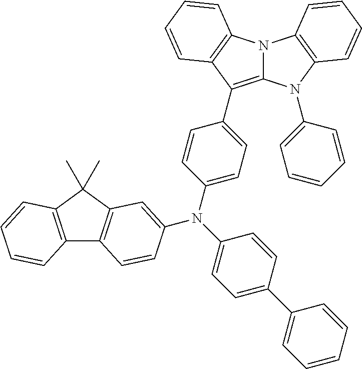 [SMILES: CC1(C)c2ccccc2-c2ccc(N(c3ccc(-c4ccccc4)cc3)c3ccc(-c4c5ccccc5n5c6ccccc6n(-c6ccccc6)c45)cc3)cc21] | 80% |
| 381 | 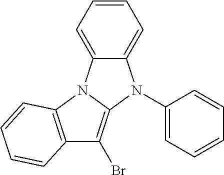 [SMILES: Brc1c2ccccc2n2c3ccccc3n(-c3ccccc3)c12] | 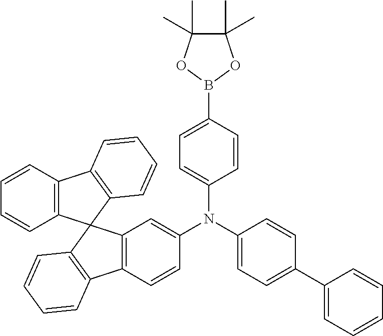 | 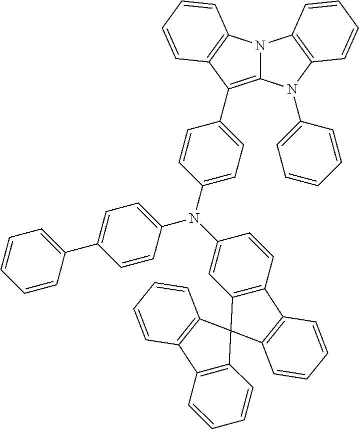 [SMILES: c1ccc(-c2ccc(N(c3ccc(-c4c5ccccc5n5c6ccccc6n(-c6ccccc6)c45)cc3)c3ccc4c(c3)C3(c5ccccc5-c5ccccc53)c3ccccc3-4)cc2)cc1] | 79% |
| 394 | 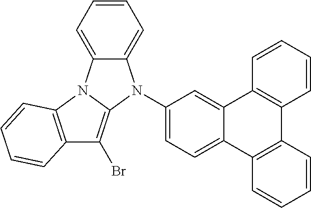 [SMILES: Brc1c2ccccc2n2c3ccccc3n(-c3ccc4c5ccccc5c5ccccc5c4c3)c12] | 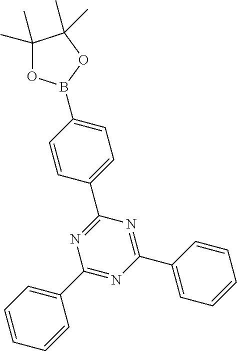 | 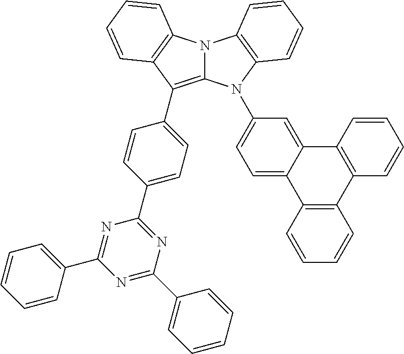 [SMILES: c1ccc(-c2nc(-c3ccccc3)nc(-c3ccc(-c4c5ccccc5n5c6ccccc6n(-c6ccc7c8ccccc8c8ccccc8c7c6)c45)cc3)n2)cc1] | 71% |
| 396 | 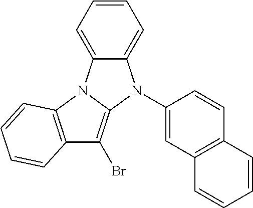 [SMILES: Brc1c2ccccc2n2c3ccccc3n(-c3ccc4ccccc4c3)c12] | 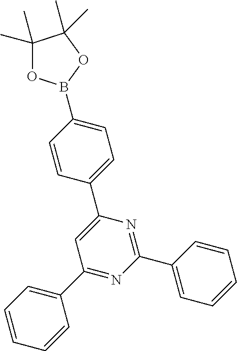 | 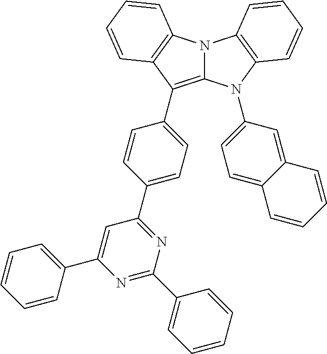 [SMILES: c1ccc(-c2cc(-c3ccc(-c4c5ccccc5n5c6ccccc6n(-c6ccc7ccccc7c6)c45)cc3)nc(-c3ccccc3)n2)cc1] | 83% |
| 401 | 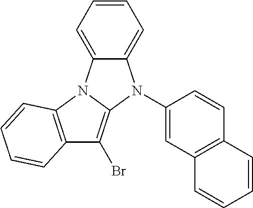 [SMILES: Brc1c2ccccc2n2c3ccccc3n(-c3ccc4ccccc4c3)c12] | 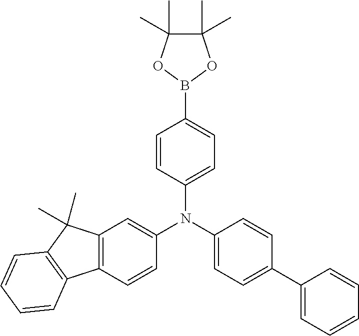 | 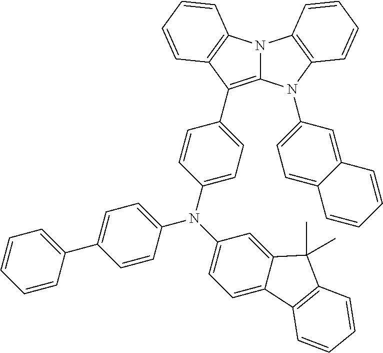 [SMILES: CC1(C)c2ccccc2-c2ccc(N(c3ccc(-c4ccccc4)cc3)c3ccc(-c4c5ccccc5n5c6ccccc6n(-c6ccc7ccccc7c6)c45)cc3)cc21] | 82% |
| 403 | 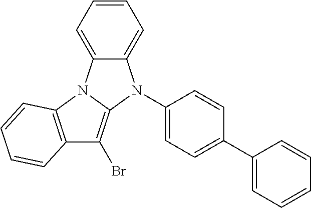 [SMILES: Brc1c2ccccc2n2c3ccccc3n(-c3ccc(-c4ccccc4)cc3)c12] | 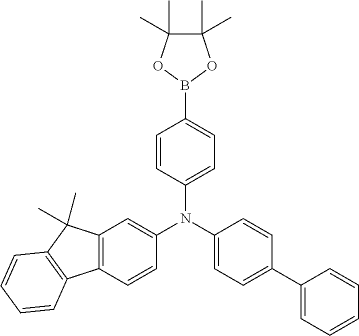 | 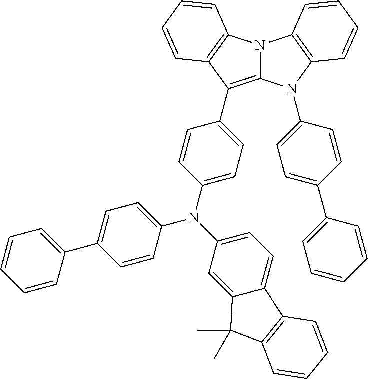 [SMILES: CC1(C)c2ccccc2-c2ccc(N(c3ccc(-c4ccccc4)cc3)c3ccc(-c4c5ccccc5n5c6ccccc6n(-c6ccc(-c7ccccc7)cc6)c45)cc3)cc21] | 79% |
| 406 | 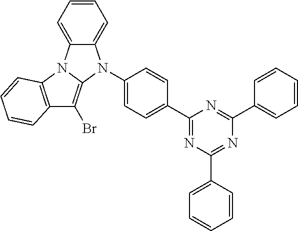 [SMILES: Brc1c2ccccc2n2c3ccccc3n(-c3ccc(-c4nc(-c5ccccc5)nc(-c5ccccc5)n4)cc3)c12] | 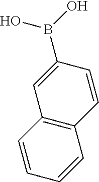 | 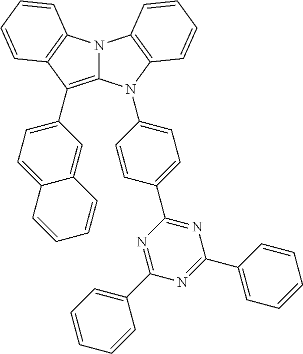 [SMILES: c1ccc(-c2nc(-c3ccccc3)nc(-c3ccc(-n4c5ccccc5n5c6ccccc6c(-c6ccc7ccccc7c6)c45)cc3)n2)cc1] | 82% |
| 421 | 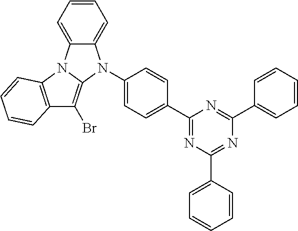 [SMILES: Brc1c2ccccc2n2c3ccccc3n(-c3ccc(-c4nc(-c5ccccc5)nc(-c5ccccc5)n4)cc3)c12] | 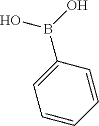 | 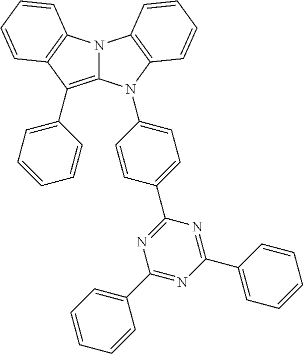 [SMILES: c1ccc(-c2nc(-c3ccccc3)nc(-c3ccc(-n4c5ccccc5n5c6ccccc6c(-c6ccccc6)c45)cc3)n2)cc1] | 80% |
| 451 | 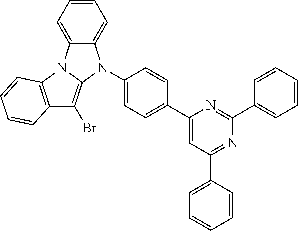 [SMILES: Brc1c2ccccc2n2c3ccccc3n(-c3ccc(-c4cc(-c5ccccc5)nc(-c5ccccc5)n4)cc3)c12] | 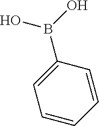 | 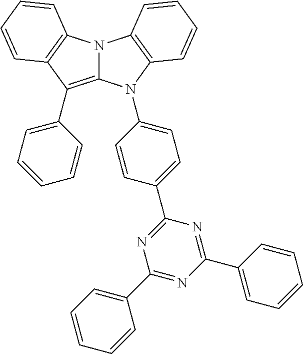 [SMILES: c1ccc(-c2nc(-c3ccccc3)nc(-c3ccc(-n4c5ccccc5n5c6ccccc6c(-c6ccccc6)c45)cc3)n2)cc1] | 79% |

Compounds other than the compounds described in Preparation Examples 1 to 6 and Tables 1 to 4 were also prepared in the same manner as the compounds described in Preparation Examples 1 to 6 and Tables 1 to 4, and the synthesis identification results are shown in the following Table 5 and Table 6.

Table 5 shows measurement values of 1H NMR (CDCl₃, 300 Mz), and Table 6 shows measurement values of FD-mass spectrometry (FD-MS: field desorption mass spectrometry).

TABLE 5

| NO | ¹H NMR (CDCl₃, 300 Mz) |
|---|---|
| 001 | 8.28 (4H, d), 7.85 (2H, d), 7.76-7.74 (3H, m), 7.51-7.25 (12H, m), |
| | 7.10 (1H, t) |
| 003 | 8.28-8.24 (3H, m), 7.85 (2H, d), 7.76-7.70 (4H, m), 7.57-7.25 |
| | (16H, m), 7.10 (1H, t) |
| 005 | 8.28 (2H, d), 7.85 (4H, d), 7.76-7.74 (3H, m), 7.52-7.25 (16H, m), |
| | 7.10 (1H, t) |
| 009 | 8.24 (2H, d), 7.85 (2H, d), 7.76-7.70 (5H, m), 7.57-7.39 (17H, m), |
| | 7.31-7.25 (3H, m), 7.10 (1H, t) |
| 011 | 8.28 (4H, d), 7.85 (2H, d), 7.76-7.74 (3H, m), 7.51-7.25 (16H, m), |
| | 7.10 (1H, t) |
| 012 | 8.28 (4H, d), 7.85 (2H, d), 7.76-7.70 (4H, m), 7.57-7.25 (15H, m), |
| | 7.10 (1H, t) |
| 014 | 8.28-8.24 (5H, m), 7.76-7.70 (5H, m), 7.57-7.31 (15H, m), 7.10 |
| | (1H, t) |
| 017 | 9.09 (2H, s), 8.49 (2H, d), 8.00-7.85 (8H, m), 7.76-7.74 (3H, m), |
| | 7.59 (4H, d), 7.48-7.25 (10H, m), 7.10 (1H, t) |
| 020 | 9.09 (2H, s), 8.49 (2H, d), 8.24 (1H, d), 8.00-7.92 (6H, m), 7.76- |
| | 7.70 (5H, m), 7.59-7.31 (13H, m), 7.10 (1H, t) |
| 021 | 8.55 (2H, d), 8.08-8.04 (4H, m), 7.95 (2H, d), 7.85 (2H, d), 7.76- |
| | 7.74 (3H, m), 7.61-7.25 (12H, m), 7.10 (1H, t) |
| 023 | 8.55 (2H, d), 8.08-8.04 (4H, m), 7.95 (2H, d), 7.85 (2H, d), 7.76- |
| | 7.74 (3H, m), 7.61-7.25 (16H, m), 7.10 (1H, t) |
| 030 | 8.52 (4H, d), 7.85 (2H, d), 7.76-7.74 (3H, m), 7.48-7.25 (14H, m), |
| | 7.10 (1H, t), 2.34 (6H, s) |
| 031 | 8.30-8.23 (5H, m), 7.79-7.74 (5H, m), 7.51-7.25 (12H, m), 7.10 |
| | (1H, t) |
| 033 | 8.30-8.23 (5H, m), 7.76-7.70 (5H, m), 7.57-7.25 (16H, m), 7.10 |
| | (1H, t) |
| 035 | 8.30-8.23 (7H, m), 7.85 (2H, d), 7.76-7.74 (3H, m), 7.52-7.25 |
| | (14H, m), 7.10 (1H, t) |
| 039 | 8.30-8.23 (4H, m), 7.76-7.70 (6H, m), 7.57-7.39 (17H, m), 7.31- |
| | 7.25 (3H, m), 7.10 (1H, t) |
| 041 | 8.30-8.23 (5H, m), 7.85-7.74 (7H, m), 7.51-7.25 (14H, m), 7.10 |
| | (1H, t) |
| 042 | 8.30-8.23 (5H, m), 7.85-7.70 (8H, m), 7.57-7.31 (13H, m), 7.10 |
| | (1H, t) |
| 043 | 8.28-8.23 (3H, m), 7.79-7.70 (7H, m), 7.57-7.25 (16H, m), 7.10 |
| | (1H, t) |
| 044 | 8.28-8.23 (3H, m), 7.79-7.70 (8H, m), 7.57-7.31 (15H, m), 7.10 |
| | (1H, t) |
| 057 | 8.55 (1H, d), 8.30-8.23 (5H, m), 8.12-8.09 (2H, m), 7.94 (1H, d), |
| | 7.79-7.74 (4H, m), 7.63 (1H, d), 7.51-7.25 (15H, m), 7.10 (1H, t) |
| 063 | 9.30 (2H, d), 9.15 (2H, s), 8.53 (2H, d), 7.76-7.70 (5H, m), 7.48- |
| | 7.25 (8H, m), 7.14-7.10 (3H, m) |
| 065 | 8.83 (1H, d), 8.38 (1H, d), 8.26-8.21 (2H, m), 8.10-8.06 (2H, m), |
| | 7.81-7.74 (4H, m), 7.60-7.31 (8H, m), 7.10 (1H, t) |
| 067 | 8.81 (2H, d), 8.30 (2H, d), 8.10-8.06 (3H, m), 7.81-7.74 (4H, m), |
| | 7.54-7.28 (11H, m), 7.10 (1H, t) |
| 068 | 8.30-8.21 (4H, m), 8.10-8.06 (3H, m), 7.81-7.74 (4H, m), 7.60- |
| | 7.31 (11H, m), 7.10 (1H, t) |
| 069 | 7.83-7.74 (11H, m), 7.48-7.39 (9H, m), 7.31 (1H, d), 7.10 (1H, t) |
| 080 | 7.87 (1H, d), 7.76-7.74 (3H, m), 7.62-7.28 (17H, m), 7.10 (1H, t), |
| | 6.75-6.69 (5H, m), 6.58 (1H, d), 1.72 (6H, s) |
| 088 | 8.07-8.02 (2H, m), 7.87 (1H, d), 7.76-7.74 (3H, m), 7.62-7.28 |
| | (14H, m), 7.10 (1H, t), 6.98 (1H, d), 6.75-6.69 (3H, m), 6.58 (1H, |
| | d), 1.72 (6H, s) |
| 092 | 8.28 (4H, d), 7.85-7.74 (5H, m), 7.65 (1H, d), 7.52-7.39 (14H, m), |
| | 7.25 (2H, d) |
| 101 | 8.28 (4H, d), 8.00-7.73 (9H, m), 7.59-7.37 (13H, m), 7.25 (2H, d) |
| 102 | 8.55 (1H, d), 8.42 (1H, d), 8.28 (4H, d), 8.08-8.04 (2H, m), 7.85- |
| | 7.74 (5H, m), 7.61-7.37 (13H, m), 7.25 (2H, d) |
| 108 | 8.30-8.23 (5H, m), 7.81-7.74 (7H, m), 7.51-7.39 (11H, m), 7.27- |
| | 7.25 (3H, m), 7.16 (1H, t) |
| 110 | 8.30-8.23 (5H, m), 8.09 (1H, s), 7.85-7.76 (5H, m), 7.52-7.41 |
| | (12H, m), 7.31-7.25 (3H, m), 7.10 (1H, t) |
| 112 | 8.30-8.23 (5H, m), 7.79-7.76 (4H, m), 7.58-7.41 (14H, m), 7.31- |
| | 7.25 (3H, m), 7.10 (1H, t) |
| 119 | 9.15 (1H, s), 8.93 (2H, d), 8.30-8.04 (9H, m), 7.88-7.74 (9H, m), |
| | 7.51-7.37 (10H, m), 7.25 (2H, d) |
| 126 | 8.83-8.81 (3H, m), 8.38 (1H, d), 8.10-8.06 (2H, m), 7.81-7.76 (3H, |
| | m), 7.68 (1H, d), 7.58-7.28 (12H, m), 7.10 (1H, t) |
| 127 | 8.81 (2H, d), 8.30 (2H, d), 8.10-8.06 (3H, m), 7.81-7.76 (3H, m), |
| | 7.54-7.28 (16H, m), 7.10 (1H, t) |
| 132 | 8.56-8.55 (2H, m), 8.42 (1H, d), 8.08-8.04 (2H, m), 7.85-7.74 (5H, |
| | m), 7.61-7.37 (13H, m), 7.25-7.22 (4H, m) |
| 140 | 8.09 (1H, s), 7.87-7.76 (4H, m), 7.62 (1H, d), 7.55-7.31 (19H, m), |
| | 7.10 (1H, t), 6.75-6.69 (5H, m), 6.58 (1H, d), 1.72 (6H, s) |
| 141 | 7.87 (1H, d), 7.79-7.75 (4H, m), 7.68-7.62 (2H, m), 7.55-7.10 |
| | (27H, m), 6.75-6.69 (5H, m), 6.58 (1H, d) |
| 151 | 8.28 (4H, d), 8.01-7.96 (2H, m), 7.85 (2H, d), 7.76 (1H, d), 7.53- |
| | 7.41 (9H, m), 7.31-7.25 (3H, m), 7.10 (1H, t) |
| 152 | 8.28-8.24 (5H, m), 8.01-7.96 (2H, m), 7.76-7.70 (2H, m), 7.57- |
| | 7.41 (11H, m), 7.31 (1H, d), 7.10 (1H, t) |
| 153 | 8.28-8.24 (3H, m), 8.01-7.96 (2H, m), 7.85 (2H, d), 7.76-7.70 (2H, |
| | m), 7.57-7.41 (13H, m), 7.31-7.25 (3H, m), 7.10 (1H, t) |
| 161 | 8.28 (4H, d), 8.01-7.96 (2H, m), 7.85 (2H, d), 7.76 (1H, d), 7.53- |
| | 7.41 (9H, m), 7.31-7.25 (7H, m), 7.10 (1H, t) |
| 165 | 9.09 (2H, d), 8.49 (2H, d), 8.01-7.85 (10H, m), 7.76 (1H, d), 7.59- |
| | 7.48 (7H, m), 7.31-7.25 (3H, m), 7.10 (1H, t) |
| 173 | 8.55 (2H, d), 8.08-7.95 (8H, m), 7.85 (2H, d), 7.76 (1H, d), 7.61- |
| | 7.48 (9H, m), 7.31-7.25 (7H, m), 7.10 (1H, t) |
| 177 | 8.55 (1H, d), 8.28 (3H, d), 8.12-8.09 (2H, m), 8.01-7.94 (3H, m), |
| | 7.85 (2H, d), 7.76 (1H, d), 7.63 (1H, d), 7.53-7.25 (15H, m), 7.10 |
| | (1H, t) |
| 181 | 8.30-8.23 (5H, m), 8.01-7.96 (2H, m), 7.79-7.76 (3H, m), 7.53- |
| | 7.41 (9H, m), 7.31-7.25 (3H, m), 7.10 (1H, t) |
| 187 | 8.30 (4H, d), 8.23 (1H, s), 8.01-7.96 (2H, m), 7.85 (4H, d), 7.76 |
| | (1H, d), 7.53-7.41 (13H, m), 7.31-7.25 (5H, m), 7.10 (1H, t) |
| 191 | 8.30-8.23 (5H, m), 8.01-7.96 (2H, m), 7.85-7.76 (5H, m), 7.53- |
| | 7.41 (9H, m), 7.31-7.25 (5H, m), 7.10 (1H, t) |
| 207 | 8.55 (1H, d), 8.30-8.23 (5H, m), 8.12-8.09 (2H, m), 8.01-7.94 (3H, |
| | m), 7.79-7.76 (2H, m), 7.63 (1H, d), 7.53-7.25 (15H, m), 7.10 (1H, |
| | t) |
| 211 | 8.30-8.28 (4H, m), 8.16 (1H, d), 8.01-7.96 (2H, m), 7.84-7.76 (3H, |
| | m), 7.58-7.41 (7H, m), 7.31-7.25 (3H, m), 7.10 (1H, t) |
| 215 | 8.83 (1H, d), 8.38 (1H, d), 8.26-8.21 (2H, m), 8.10-7.96 (4H, m), |
| | 7.81-7.76 (2H, m), 7.60-7.48 (6H, m), 7.35-7.31 (2H, m), 7.10 (1H, |
| | t) |
| 217 | 8.81 (2H, d), 8.30 (2H, d), 8.10-7.96 (5H, m), 7.81-7.76 (2H, m), |
| | 7.54-7.47 (6H, m), 7.35-7.28 (5H, m), 7.10 (1H, t) |
| 218 | 8.30-8.21 (4H, m), 8.10-7.96 (5H, m), 7.81-7.76 (2H, m), 7.60- |
| | 7.47 (8H, m), 7.35-7.31 (3H, m), 7.10 (1H, t) |
| 219 | 8.01-7.96 (2H, m), 7.83-7.76 (9H, m), 7.53-7.45 (9H, m), 7.31 (1H, |
| | d), 7.10 (1H, t) |
| 230 | 8.01-7.96 (2H, m), 7.87 (1H, d), 7.76 (1H, d), 7.62-7.28 (17H, m), |
| | 7.10 (1H, t), 6.75-6.69 (5H, m), 6.58 (1H, d), 1.72 (6H, s) |
| 238 | 8.02-7.96 (4H, m), 7.87 (1H, d), 7.76 (1H, d), 7.62-7.48 (10H, m), |
| | 7.38-7.28 (4H, m), 7.10 (1H, t), 6.98 (1H, d), 6.75-6.69 (3H, m), |
| | 6.58 (1H, d), 1.72 (6H, s) |
| 243 | 8.28 (4H, m), 8.01-7.96 (2H, m), 7.85-7.79 (5H, m), 7.53-7.41 |
| | (11H, m), 7.27-7.25 (3H, m), 7.16 (1H, t) |
| 244 | 8.28 (4H, d), 7.97 (1H, d), 7.85 (2H, d), 7.77-7.76 (2H, m), 7.59- |
| | 7.41 (11H, m), 7.31-7.19 (5H, m), 7.10 (1H, t) |
| 251 | 8.28 (4H, d), 8.01-7.81 (8H, m), 7.73 (1H, d), 7.59-7.37 (13H, m), |
| | 7.25 (2H, d) |
| 260 | 8.45 (1H, s), 8.30-8.23 (5H, m), 8.07 (1H, d), 7.79-7.76 (4H, m), |
| | 7.52-7.41 (12H, m), 7.31-7.25 (3H, m), 7.10 (1H, t) |
| 262 | 8.30-8.23 (5H, m), 7.92 (1H, d), 7.79-7.76 (4H, m), 7.59-7.41 |
| | (13H, m), 7.31-7.25 (3H, m), 7.10 (1H, t) |
| 277 | 8.81 (2H, d), 8.30 (2H, d), 8.10-8.06 (3H, m), 7.92 (1H, d), 7.81- |
| | 7.76 (3H, m), 7.59-7.28 (15H, m), 7.10 (1H, t) |
| 281 | 8.01-7.92 (5H, m), 7.83-7.73 (10H, m), 7.59-7.37 (13H, m), 7.25 |
| | (4H, s) |
| 290 | 8.45 (1H, s), 8.07 (1H, d), 7.87 (1H, d), 7.77-7.76 (2H, m), 7.62- |
| | 7.31 (20H, m), 7.10 (1H, t), 6.75-6.69 (5H, m), 6.58 (1H, d), 1.72 |
| | (6H, s) |
| 291 | 8.34 (1H, s), 7.87-7.75 (6H, m), 7.62-7.10 (27H, m), 6.75-6.69 |
| | (5H, m), 6.58 (1H, d) |
| 301 | 8.56 (2H, d), 8.28 (4H, d), 7.85 (2H, d), 7.76 (1H, d), 7.58-7.41 |
| | (12H, m), 7.31-7.22 (5H, m), 7.10 (1H, t) |
| 314 | 8.56 (2H, d), 8.28-8.24 (5H, m), 7.76-7.70 (3H, m), 7.58-7.41 |
| | (17H, m), 7.31 (1H, d), 7.22 (2H, d), 7.10 (1H, t) |
| 315 | 9.09 (2H, d), 8.56-8.49 (4H, m), 8.00-7.85 (8H, m), 7.76 (1H, d), |
| | 7.59-7.48 (10H, m), 7.31-7.22 (5H, m), 7.10 (1H, t) |
| 329 | 8.56-8.52 (6H, m), 7.85 (2H, d), 7.76 (1H, d), 7.58-7.45 (6H, m), |
| | 7.31-7.22 (9H, m), 7.10 (1H, t), 2.34 (6H, s) |
| 331 | 8.56 (2H, d), 8.30-8.23 (5H, m), 7.79-7.76 (3H, m), 7.58-7.41 |
| | (12H, m), 7.31-7.22 (5H, m), 7.10 (1H, t) |
| 341 | 8.56 (2H, d), 8.30-8.23 (5H, m), 7.85-7.76 (5H, m), 7.58-7.41 |
| | (12H, m), 7.31-7.22 (7H, m), 7.10 (1H, t) |
| 342 | 8.56 (2H, d), 8.30-8.23 (5H, m), 7.85-7.76 (6H, m), 7.58-7.41 |
| | (15H, m), 7.31 (1H, d), 7.22 (2H, d), 7.10 (1H, t) |
| 357 | 8.56 (2H, d), 8.30-8.23 (5H, m), 8.12-8.09 (2H, m), 7.94 (1H, d), |
| | 7.79-7.76 (2H, m), 7.58-7.22 (21H, m), 7.10 (1H, t) |
| 361 | 8.56 (2H, d), 8.30-8.28 (4H, m), 8.16 (1H, d), 7.84-7.76 (3H, m), |
| | 7.58-7.41 (10H, m), 7.31-7.22 (5H, m), 7.10 (1H, t) |
| 373 | 8.56 (3H, d), 8.28 (2H, d), 7.79-7.41 (15H, m), 7.31-7.22 (5H, m), |
| | 7.10 (1H, t) |
| 380 | 8.56 (2H, d), 7.87 (1H, d), 7.76 (1H, d), 7.62-7.22 (22H, m), 7.10 |
| | (1H, t), 6.75-6.69 (5H, m), 6.58 (1H, d), 1.72 (6H, s) |
| 381 | 8.56 (2H, d), 7.87 (1H, d), 7.76-7.75 (3H, m), 7.62-7.10 (29H, m), |
| | 6.73-6.69 (5H, m), 6.58 (1H, d) |
| 394 | 8.93-8.90 (3H, m), 8.56 (2H, d), 8.28 (4H, d), 8.12-8.10 (3H, m), |
| | 7.88-7.76 (8H, m), 7.51-7.41 (7H, m), 7.31-7.22 (5H, m), 7.10 (1H, |
| | t) |
| 396 | 8.56 (2H, d), 8.30-8.23 (5H, m), 8.00 (3H, d), 7.83-7.76 (4H, m), |
| | 7.59-7.22 (15H, m), 7.10 (1H, t) |
| 401 | 8.56 (2H, d), 8.00 (3H, d), 7.87-7.83 (2H, m), 7.76 (1H, d), 7.59- |
| | 7.22 (20H, m), 7.10 (1H, t), 6.75-6.69 (5H, m), 6.58 (1H, d), 1.72 |
| | (6H, s) |
| 403 | 8.56 (2H, d), 7.87 (1H, d), 7.79-7.76 (3H, m), 7.68-7.22 (24H, m), |
| | 7.10 (1H, t), 6.75-6.69 (5H, m), 6.58 (1H, d), 1.72 (6H, s) |
| 406 | 8.56 (2H, d), 8.28 (4H, d), 8.00-7.92 (3H, m), 7.79-7.41 (16H, m), |
| | 7.31 (1H, d), 7.22 (2H, d), 7.10 (1H, t) |
| 421 | 8.56 (2H, d), 8.28 (4H, d), 7.79-7.76 (3H, m), 7.68 (2H, d), 7.52- |
| | 7.41 (12H, m), 7.31 (1H, d), 7.22 (2H, d), 7.10 (1H, t) |
| 451 | 8.56 (2H, d), 8.28-8.23 (3H, m), 7.79-7.76 (5H, m), 7.68 (2H, d), |
| | 7.52-7.41 (12H, m), 7.31 (1H, d), 7.22 (2H, d), 7.10 (1H, t) |

TABLE 6

| Com- | | Com- | |
|---|---|---|---|
| pound | FD-MS | pound | FD-MS |
| 001 | m/z = 514.58 | 003 | m/z = 590.67 |
| | (C₃₅H₂₂N₄O = 514.18) | | (C₄₁H₂₆N₄O = 590.21) |
| 005 | m/z = 590.67 | 009 | m/z = 666.77 |
| | (C₄₁H₂₆N₄O = 590.21) | | (C₄₇H₃₀N₄O = 666.24) |
| 011 | m/z = 590.67 | 012 | m/z = 590.67 |
| | (C₄₁H₂₆N₄O = 590.21) | | (C₄₁H₂₆N₄O = 590.21) |
| 014 | m/z = 590.67 | 017 | m/z = 690.79 |
| | (C₄₁H₂₆N₄O = 590.21) | | (C₄₉H₃₀N₄O = 690.24) |
| 020 | m/z = 690.79 | 021 | m/z = 614.69 |
| | (C₄₉H₃₀N₄O = 690.24) | | (C₄₃H₂₆N₄O = 614.21) |
| 023 | m/z = 690.79 | 030 | m/z = 618.72 |
| | (C₄₉H₃₀N₄O = 690.24) | | (C₄₃H₃₀N₄O = 618.24) |
| 031 | m/z = 513.59 | 033 | m/z = 589.68 |
| | (C₃₆H₂₃N₃O = 513.18) | | (C₄₂H₂₇N₃O = 589.22) |
| 035 | m/z = 589.68 | 039 | m/z = 665.78 |
| | (C₄₂H₂₇N₃O = 589.22) | | (C₄₈H₃₁N₃O = 665.25) |
| 041 | m/z = 589.68 | 042 | m/z = 589.68 |
| | (C₄₂H₂₇N₃O = 589.22) | | (C₄₂H₂₇N₃O = 589.22) |
| 043 | m/z = 589.68 | 044 | m/z = 589.68 |
| | (C₄₂H₂₇N₃O = 589.22) | | (C₄₂H₂₇N₃O = 589.22) |
| 057 | m/z = 678.78 | 063 | m/z = 514.58 |
| | (C₄₈H₃₀N₄O = 678.24) | | (C₃₅H₂₂N₄O = 514.18) |
| 065 | m/z = 461.51 | 067 | m/z = 537.61 |
| | (C₃₂H₁₈N₃O = 461.15) | | (C₃₈H₂₃N₃O = 537.18) |
| 068 | m/z = 537.61 | 069 | m/z = 483.50 |
| | (C₃₈H₂₃N₃O = 537.18) | | (C₃₂H₂₂NO₂P = 483.14) |
| 080 | m/z = 642.79 | 088 | m/z = 616.75 |
| | (C₄₇H₃₄N₂O = 642.27) | | (C₄₅H₃₂N₂O = 616.25) |
| 092 | m/z = 590.67 | 101 | m/z = 640.73 |
| | (C₄₁H₂₆N₄O = 590.21) | | (C₄₅H₂₈N₄O = 640.23) |
| 102 | m/z = 640.73 | 108 | m/z = 589.68 |
| | (C₄₅H₂₈N₄O = 640.23) | | (C₄₂H₂₇N₃O = 589.22) |
| 110 | m/z = 589. 68 | 112 | m/z = 589.68 |
| | (C₄₂H₂₇N₃O = 589.22) | | (C₄₂H₂₇N₃O = 589.22) |
| 119 | m/z = 739.86 | 126 | m/z = 537.61 |
| | (C₅₄H₃₃N₃O = 739.26) | | (C₃₈H₂₃N₃O = 537.18) |
| 127 | m/z = 613.70 | 132 | m/z = 601.69 |
| | (C₄₄H₂₇N₃O = 613.22) | | (C₄₃H₂₇N₃O = 601.22) |
| 140 | m/z = 718.88 | 141 | m/z = 841.00 |
| | (C₅₃H₃₈N₂O = 718.30) | | (C₆₃H₄₀N₂O = 840.31) |
| 151 | m/z = 530.64 | 152 | m/z = 530.64 |
| | (C₃₅H₂₂N₄S = 530.16) | | (C₃₅H₂₂N₄S = 530.16) |
| 153 | m/z = 606.74 | 161 | m/z = 606.74 |
| | (C₄₁H₂₆N₄S = 606.19) | | (C₄₁H₂₆N₄S = 606.19) |
| 165 | m/z = 630.76 | 173 | m/z = 706.85 |
| | (C₄₃H₂₆N₄S = 630.19) | | (C₄₉H₃₀N₄S = 706.22) |
| 177 | m/z = 695.83 | 181 | m/z = 529.65 |
| | (C₄₇H₂₉N₅S = 695.21) | | (C₃₆H₂₃N₃S = 529.16) |
| 187 | m/z = 681.84 | 191 | m/z = 605.75 |
| | (C₄₈H₃₁N₃S = 681.22) | | (C₄₂H₂₇N₃S = 605.19) |
| 207 | m/z = 694.84 | 211 | m/z = 503.62 |
| | (C₄₈H₃₀N₄S = 694.22) | | (C₃₄H₂₁N₃S = 503.15) |
| 215 | m/z = 477.58 | 217 | m/z = 553.67 |
| | (C₃₂H₁₉N₃S = 477.13) | | (C₃₈H₂₃N₃S = 553.16) |
| 218 | m/z = 553.67 | 219 | m/z = 499.56 |
| | (C₃₈H₂₃N₃S = 553.16) | | (C₃₂H₂₂NOPS = 499.12) |
| 230 | m/z = 658.85 | 238 | m/z = 632.81 |
| | (C₄₇H₃₄N₂S = 658.24) | | (C₄₅H₃₂N₂S = 632.23) |
| 243 | m/z = 606.74 | 244 | m/z = 606.74 |
| | (C₄₁H₂₆N₄S = 606.19) | | (C₄₁H₂₆N₄S = 606.19) |
| 251 | m/z = 656.80 | 260 | m/z = 605.75 |
| | (C₄₅H₂₈N₄S = 656.20) | | (C₄₂H₂₇N₃S = 605.19) |
| 262 | m/z = 605.75 | 277 | m/z = 629.77 |
| | (C₄₂H₂₇N₃S = 605.19) | | (C₄₄H₂₇N₃S = 629.19) |
| 281 | m/z = 701.81 | 290 | m/z = 734.95 |
| | (C₄₈H₃₂NOPS = 701.19) | | (C₅₃H₃₈N₂S = 734.28) |
| 291 | m/z = 857.07 | 301 | m/z = 589.69 |
| | (C₆₃H₄₀N₂S = 856.29) | | (C₄₁H₂₇N₅ = 589.23) |
| 314 | m/z = 665.78 | 315 | m/z = 689.80 |
| | (C₄₇H₃₁N₅ = 665.26) | | (C₄₉H₃₁N₅ = 689.26) |
| 329 | m/z = 617.74 | 331 | m/z = 588.70 |
| | (C₄₃H₃₁N₅ = 617.26) | | (C₄₂H₂₈N₄ = 588.23) |
| 341 | m/z = 664.79 | 342 | m/z = 664.79 |
| | (C₄₈H₃₂N₄ = 664.26) | | (C₄₈H₃₂N₄ = 664.26) |
| 357 | m/z = 753.89 | 361 | m/z = 562.66 |
| | (C₅₄H₃₅N₅ = 753.29) | | (C₄₀H₂₆N₄ = 562.22) |
| 373 | m/z = 550.65 | 380 | m/z = 717.90 |
| | (C₃₉H₂₆N₄ = 550.22) | | (C₅₃H₃₉N₃ = 717.31) |
| 381 | m/z = 840.02 | 394 | m/z = 739.86 |
| | (C₆₃H₄₂N₃ = 839.33) | | (C₅₃H₃₃N₅ = 739.27) |
| 396 | m/z = 638.76 | 401 | m/z = 767.96 |
| | (C₄₆H₃₀N₄ = 638 .25) | | (C₅₇H₄₁N₃ = 767.33) |
| 403 | m/z = 793.99 | 406 | m/z = 639.75 |
| | (C₅₉H₄₃N₃ = 793.35) | | (C₄₅H₂₉N₅ = 639.24 ) |
| 421 | m/z = 589.69 | 451 | m/z = 588.70 |
| | (C₄₁H₂₇N₅ = 589.23) | | (C₄₂H₂₈N₄ = 588.23) |

### EXPERIMENTAL EXAMPLE

### Experimental Example 1

Manufacture of Organic Light Emitting Device A transparent indium tin oxide (ITO) electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, an ITO substrate was installed in a substrate folder of a vacuum deposition apparatus, and the following 4,4′,4″-tris(N,N-(2-naphthyl)-phenylamino)triphenylamine (2-TNATA) was introduced to a cell in the vacuum deposition apparatus.

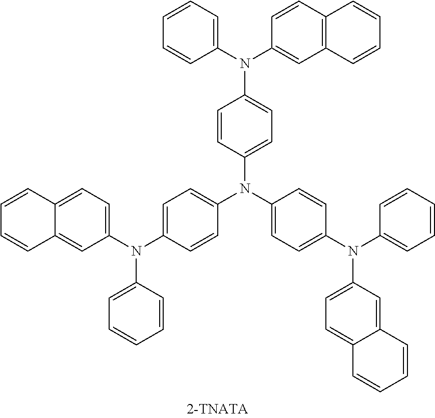 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate.

To another cell in the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

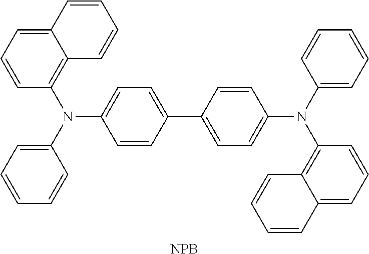 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

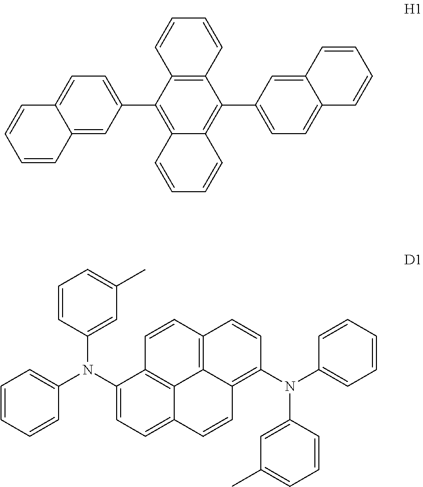

Subsequently, a compound of the following Table 7 was deposited to a thickness of 300 Å as an electron transfer layer.

As an electron injection layer, lithium fluoride (LiF) was deposited to a thickness of 10 Å, and an Al cathode was employed to a thickness of 1,000 Å, and as a result, an OLED was manufactured.

Meanwhile, all the organic compounds required to manufacture the OLED were vacuum sublimation purified under 10⁻⁶ torr to 10⁻⁸ torr by each material to be used in the OLED manufacture.

Results of measuring driving voltage, light emission efficiency, color coordinate (CIE) and lifetime of the blue organic light emitting devices manufactured according to the present disclosure are as shown in Table 7.

TABLE 7

| | | | Light | | |
|---|---|---|---|---|---|
| | | Driving | Emission | | |
| | | Voltage | Efficiency | | Lifetime |
| | Compound | (V) | (cd/A) | CIE (x, y) | (T95) |
| Comparative | El | 5.56 | 5.91 | (0.134, 0.100) | 40 |
| Example 1-1 | | | | | |
| Comparative | BBQB | 5.50 | 6.10 | (0.134, 0.101) | 42 |
| Example 1-2 | | | | | |
| Comparative | TBQB | 5.51 | 6.15 | (0.134, 0.102) | 43 |
| Example 1-3 | | | | | |
| Comparative | E2 | 5.66 | 5.87 | (0.134, 0.101) | 41 |
| Example 1-4 | | | | | |
| Comparative | E3 | 5.74 | 5.95 | (0.134, 0.103) | 39 |
| Example 1-5 | | | | | |
| Example 1 | 001 | 5.21 | 6.75 | (0.134, 0.101) | 41 |
| Example 2 | 003 | 5.16 | 6.84 | (0.134, 0.102) | 45 |
| Example 3 | 005 | 5.14 | 6.70 | (0.134, 0.101) | 40 |
| Example 4 | 009 | 5.32 | 6.76 | (0.134, 0.103) | 47 |
| Example 5 | 011 | 4.43 | 7.11 | (0.134, 0.102) | 60 |
| Example 6 | 012 | 5.13 | 6.59 | (0.134, 0.101) | 42 |
| Example 7 | 014 | 5.33 | 6.52 | (0.134, 0.102) | 41 |
| Example 8 | 017 | 5.32 | 6.43 | (0.134, 0.101) | 43 |
| Example 9 | 020 | 5.40 | 6.33 | (0.134, 0.101) | 43 |
| Example 10 | 021 | 4.52 | 7.01 | (0.134, 0.100) | 57 |
| Example 11 | 023 | 5.37 | 6.35 | (0.134, 0.101) | 46 |
| Example 12 | 030 | 5.38 | 6.61 | (0.134, 0.100) | 48 |
| Example 13 | 031 | 5.27 | 6.72 | (0.134, 0.100) | 47 |
| Example 14 | 033 | 5.48 | 6.52 | (0.134, 0.100) | 45 |
| Example 15 | 035 | 5.11 | 6.37 | (0.134, 0.100) | 41 |
| Example 16 | 039 | 5.35 | 6.68 | (0.134, 0.100) | 40 |
| Example 17 | 041 | 5.22 | 6.28 | (0.134, 0.102) | 42 |
| Example 18 | 042 | 5.12 | 6.29 | (0.134, 0.101) | 40 |
| Example 19 | 043 | 5.09 | 6.90 | (0.134, 0.102) | 45 |
| Example 20 | 044 | 5.42 | 6.88 | (0.134, 0.100) | 45 |
| Example 21 | 057 | 4.41 | 7.17 | (0.134, 0.103) | 53 |
| Example 22 | 063 | 5.63 | 5.67 | (0.134, 0.100) | 35 |
| Example 23 | 069 | 5.63 | 5.86 | (0.134, 0.102) | 80 |
| Example 24 | 092 | 5.30 | 6.57 | (0.134, 0.100) | 41 |
| Example 25 | 101 | 5.37 | 6.45 | (0.134, 0.101) | 42 |
| Example 26 | 102 | 5.22 | 6.41 | (0.134, 0.100) | 44 |
| Example 27 | 110 | 5.30 | 6.72 | (0.134, 0.100) | 46 |
| Example 28 | 112 | 5.28 | 6.35 | (0.134, 0.101) | 44 |
| Example 29 | 119 | 5.38 | 6.41 | (0.134, 0.100) | 45 |
| Example 30 | 132 | 5.27 | 6.49 | (0.134, 0.100) | 41 |
| Example 31 | 151 | 4.79 | 7.08 | (0.134, 0.100) | 50 |
| Example 32 | 152 | 5.05 | 6.79 | (0.134, 0.100) | 40 |
| Example 33 | 153 | 5.35 | 6.68 | (0.134, 0.100) | 47 |
| Example 34 | 161 | 4.82 | 7.11 | (0.134, 0.102) | 51 |
| Example 35 | 165 | 5.12 | 6.44 | (0.134, 0.101) | 47 |
| Example 36 | 173 | 5.09 | 6.84 | (0.134, 0.102) | 45 |
| Example 37 | 177 | 5.72 | 5.88 | (0.134, 0.100) | 77 |
| Example 38 | 181 | 5.21 | 6.45 | (0.134, 0.103) | 41 |
| Example 39 | 187 | 5.38 | 6.60 | (0.134, 0.100) | 43 |
| Example 40 | 191 | 4.90 | 7.06 | (0.134, 0.102) | 50 |
| Example 41 | 207 | 5.27 | 6.42 | (0.134, 0.100) | 44 |
| Example 42 | 211 | 5.31 | 6.21 | (0.134, 0.100) | 44 |
| Example 43 | 219 | 5.72 | 5.39 | (0.134, 0.100) | 82 |
| Example 44 | 243 | 5.29 | 6.68 | (0.134, 0.100) | 46 |
| Example 45 | 244 | 4.93 | 7.15 | (0.134, 0.102) | 55 |
| Example 46 | 251 | 5.12 | 6.20 | (0.134, 0.101) | 49 |
| Example 47 | 260 | 5.06 | 6.66 | (0.134, 0.102) | 43 |
| Example 48 | 262 | 5.30 | 6.71 | (0.134, 0.100) | 46 |
| Example 49 | 281 | 5.66 | 5.45 | (0.134, 0.103) | 81 |
| Example 50 | 301 | 4.88 | 7.21 | (0.134, 0.100) | 53 |
| Example 51 | 314 | 5.30 | 6.72 | (0.134, 0.100) | 46 |
| Example 52 | 315 | 5.27 | 6.35 | (0.134, 0.101) | 43 |
| Example 53 | 329 | 5.31 | 6.41 | (0.134, 0.100) | 46 |
| Example 54 | 331 | 5.27 | 6.42 | (0.134, 0.100) | 41 |
| Example 55 | 341 | 5.28 | 6.91 | (0.134, 0.100) | 43 |
| Example 56 | 342 | 5.12 | 6.89 | (0.134, 0.100) | 49 |
| Example 57 | 357 | 4.85 | 7.18 | (0.134, 0.100) | 53 |
| Example 58 | 361 | 5.22 | 6.60 | (0.134, 0.102) | 45 |
| Example 59 | 373 | 5.12 | 6.75 | (0.134, 0.101) | 45 |
| Example 60 | 394 | 5.09 | 6.77 | (0.134, 0.102) | 42 |
| Example 61 | 396 | 5.22 | 6.62 | (0.134, 0.100) | 47 |
| Example 62 | 406 | 5.21 | 6.45 | (0.134, 0.103) | 43 |
| Example 63 | 421 | 5.35 | 6.56 | (0.134, 0.100) | 45 |
| Example 64 | 451 | 5.30 | 6.76 | (0.134, 0.102) | 44 |

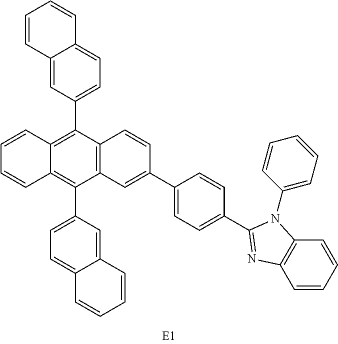 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

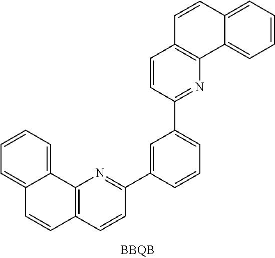 [SMILES: c1cc(-c2ccc3ccc4ccccc4c3n2)cc(-c2ccc3ccc4ccccc4c3n2)c1]

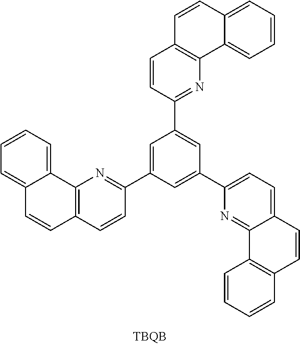 [SMILES: c1ccc2c(c1)ccc1ccc(-c3cc(-c4ccc5ccc6ccccc6c5n4)cc(-c4ccc5ccc6ccccc6c5n4)c3)nc12]

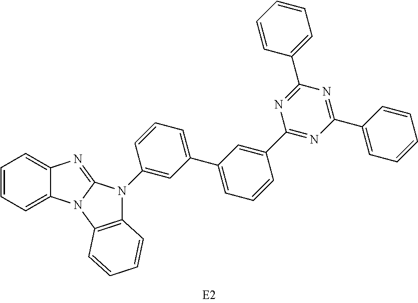 [SMILES: c1ccc(-c2nc(-c3ccccc3)nc(-c3cccc(-c4cccc(-n5c6ccccc6n6c7ccccc7nc56)c4)c3)n2)cc1]

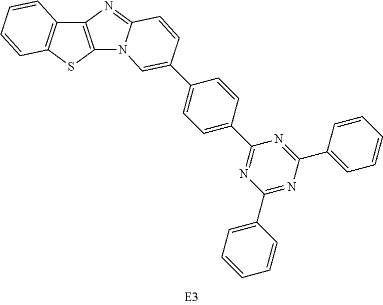 [SMILES: c1ccc(-c2nc(-c3ccccc3)nc(-c3ccc(-c4ccc5nc6c7ccccc7sc6n5c4)cc3)n2)cc1]

As seen from the results of Table 7, the organic light emitting device using the electron transfer layer material of the blue organic light emitting device of the present disclosure had lower driving voltage and significantly improved light emission efficiency and lifetime compared to Comparative Example 1-1 to Comparative Example 1-5. Particularly, it was identified that Compounds 011, 021, 057, 151, 191, 207, 244, 301 and 357 were superior in all aspects of driving, efficiency and lifetime.

Such a result is considered to be due to the fact that, when using the disclosed compound having proper length and strength, and flatness as an electron transfer layer, a compound in an excited state is made by receiving electrons under a specific condition, and particularly when an excited state is formed in the hetero-skeleton site of the compound, excited energy moves to a stable state before the excited hetero-skeleton site goes through other reactions, and as a result, the relatively stabilized compound is capable of efficiently transferring electrons without the compound being decomposed or destroyed. For reference, those that are stable when excited are aryl or acene-based compounds or polycyclic hetero-compounds. Accordingly, it is considered that excellent results in all aspects of driving, efficiency and lifetime were obtained by the compound of the present disclosure enhancing enhanced electron-transfer properties or improved stability.

### Experimental Example 2

1) Manufacture of Organic Light Emitting Device

A transparent ITO electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

On the transparent ITO electrode (anode), an organic material was formed in a 2-stack white organic light emitting device (WOLED) structure. As for a first stack, TAPC was thermal vacuum deposited first to a thickness of 300 Å to form a hole transfer layer. After forming the hole transfer layer, a light emitting layer was thermal vacuum deposited thereon as follows. The light emitting layer was deposited to a thickness of 300 Å by doping FIrpic as a blue phosphorescent dopant to TCz1, a host, by 8%. After forming an electron transfer layer to 400 Å using TmPyPB, a charge generation layer was formed to a thickness of 100 Å by doping Cs₂CO₃ to a compound described in the following Table 8 by 20%.

As for a second stack, MoO₃ was thermal vacuum deposited first to a thickness of 50 Å to form a hole injection layer. A hole transfer layer, a common layer, was formed by doping MoO₃ to TAPC by 20% to 100 Å and then depositing TAPC to 300 Å. A light emitting layer was deposited thereon to 300 Å by doping Ir(ppy)₃, a green phosphorescent dopant, to TCz1, a host, by 8%, and an electron transfer layer was formed to 600 Å using TmPyPB. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic light emitting device was manufactured.

Meanwhile, all the organic compounds required to manufacture the OLED were vacuum sublimation purified under 10⁻⁶ torr to 10⁻⁸ torr by each material to be used in the OLED manufacture.

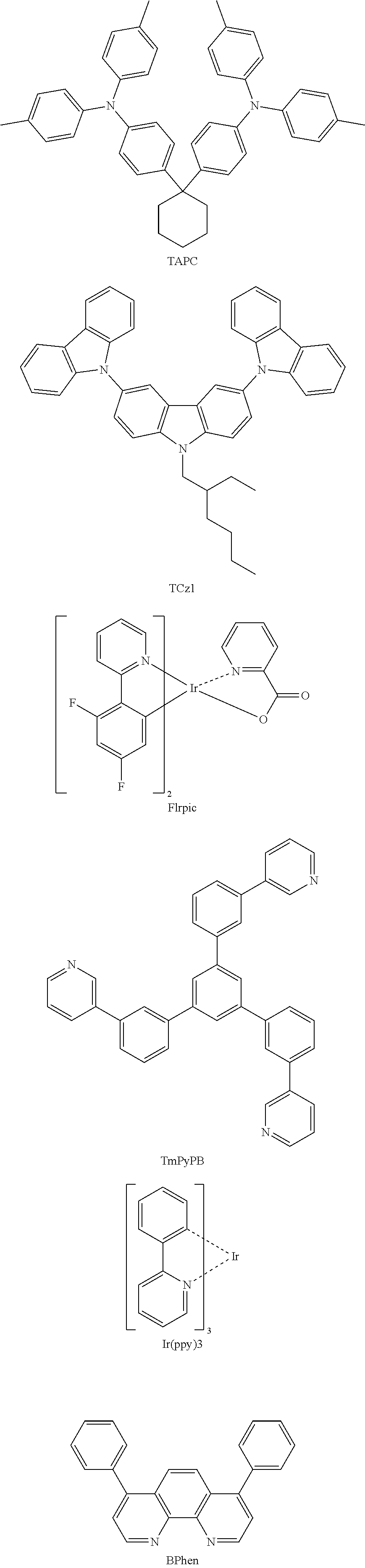

Results of measuring driving voltage, light emission efficiency, color coordinate (CIE) and lifetime of the white organic light emitting devices manufactured according to the present disclosure are as shown in the following Table 8.

TABLE 8

| | | | Light | | |
|---|---|---|---|---|---|
| | | Driving | Emission | | Life- |
| | Com- | Voltage | Efficiency | | time |
| | pound | (V) | (cd/A) | CIE (x, y) | (T95) |
| Comparative | BPhen | 7.54 | 54.23 | (0.213, 0.430) | 25 |
| Example 2 | | | | | |
| Example 65 | 065 | 6.84 | 60.44 | (0.212, 0.421) | 35 |
| Example 66 | 067 | 6.44 | 66.32 | (0.211, 0.433) | 40 |
| Example 67 | 068 | 6.56 | 59.68 | (0.214, 0.439) | 33 |
| Example 68 | 126 | 6.27 | 67.99 | (0.212, 0.424) | 53 |
| Example 69 | 127 | 6.82 | 59.18 | (0.214, 0.437) | 34 |
| Example 70 | 215 | 6.73 | 62.44 | (0.212, 0.426) | 37 |
| Example 71 | 217 | 6.72 | 57.18 | (0.214, 0.437) | 36 |
| Example 72 | 218 | 6.34 | 68.49 | (0.213, 0.424) | 49 |
| Example 73 | 277 | 6.37 | 67.41 | (0.213, 0.423) | 46 |

As seen from the results of Table 8, it was identified that the organic electroluminescent device using the charge generation layer material of the 2-stack white organic electroluminescent device of the present disclosure had lower driving voltage and improved light emission efficiency compared to Comparative Example 2. Such a result is due to the fact that the compound of the present disclosure used as an N-type charge generation layer formed with the disclosed skeleton having proper length and strength, and flatness and a proper hetero-compound capable of binding to metals forms a gap state in the N-type charge generation layer by doping an alkali metal or an alkaline earth metal thereto, and electrons produced from a P-type charge generation layer are readily injected into an electron transfer layer through the gap state produced in the N-type charge generation layer. Accordingly, the P-type charge generation layer may favorably inject and transfer electrons to the N-type charge generation layer, and as a result, driving voltage was lowered, and efficiency and lifetime were improved in the organic light emitting device.

### Experimental Example 3

1) Manufacture of Organic Light Emitting Device

A transparent ITO electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, an ITO substrate was installed in a substrate folder of a vacuum deposition apparatus, and the following 4,4′,4″-tris(N,N-(2-naphthyl)-phenylamino)triphenylamine (2-TNATA) was introduced to a cell in the vacuum deposition apparatus.

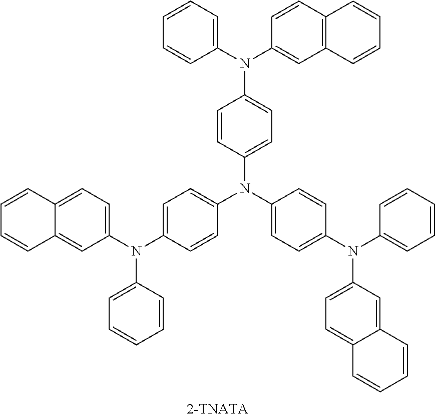 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate.

To another cell in the vacuum deposition apparatus, a compound described in the following Table 9 was introduced, and evaporated by applying a current to the cell to deposit a hole transfer layer having a thickness of 300 Å on the hole injection layer.

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

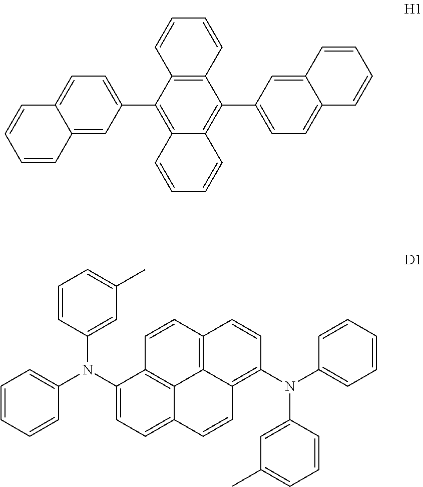

Subsequently, Compound E1 was deposited to a thickness of 300 Å as an electron transfer layer.

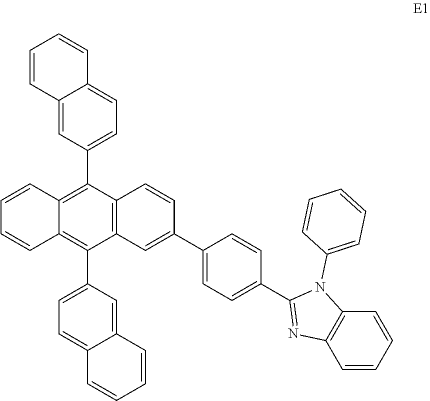 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

As an electron injection layer, lithium fluoride (LiF) was deposited to a thickness of 10 Å, and an Al cathode was employed to a thickness of 1,000 Å, and as a result, an OLED was manufactured.

Meanwhile, all the organic compounds required to manufacture the OLED were vacuum sublimation purified under 10⁻⁶ torr to 10⁻⁸ torr by each material to be used in the OLED manufacture.

Results of measuring driving voltage, light emission efficiency, color coordinate (CIE) and lifetime of the blue organic light emitting devices manufactured according to the present disclosure are as shown in Table 9.

TABLE 9

| | | | Light | | |
|---|---|---|---|---|---|
| | | Driving | Emission | | |
| | Com- | Voltage | Efficiency | | Lifetime |
| | pound | (V) | (cd/A) | CIE (x, y) | (T95) |
| Comparative | NPB | 5.56 | 5.91 | (0.134, 0.100) | 40 |
| Example 3-1 | | | | | |
| Comparative | H1 | 5.49 | 6.05 | (0.134, 0.101) | 45 |
| Example 3-2 | | | | | |
| Example 74 | 080 | 5.21 | 6.75 | (0.134, 0.101) | 47 |
| Example 75 | 088 | 5.16 | 6.84 | (0.134, 0.102) | 45 |
| Example 76 | 140 | 5.14 | 6.70 | (0.134, 0.101) | 47 |
| Example 77 | 141 | 5.32 | 6.76 | (0.134, 0.103) | 47 |
| Example 78 | 230 | 5.33 | 6.51 | (0.134, 0.102) | 51 |
| Example 79 | 238 | 5.13 | 6.49 | (0.134, 0.101) | 47 |
| Example 80 | 290 | 5.33 | 6.42 | (0.134, 0.102) | 49 |
| Example 81 | 291 | 5.32 | 6.43 | (0.134, 0.101) | 46 |
| Example 82 | 380 | 5.40 | 6.33 | (0.134, 0.101) | 48 |
| Example 83 | 381 | 5.12 | 6.62 | (0.134, 0.100) | 50 |
| Example 84 | 401 | 5.37 | 6.35 | (0.134, 0.101) | 46 |
| Example 85 | 403 | 5.32 | 6.61 | (0.134, 0.100) | 48 |

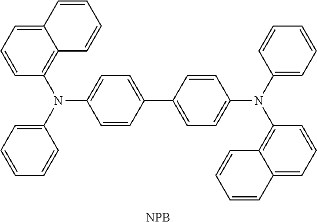 [SMILES: c1ccc(N(c2ccc(-c3ccc(N(c4ccccc4)c4cccc5ccccc45)cc3)cc2)c2cccc3ccccc23)cc1 ; "NPB"]

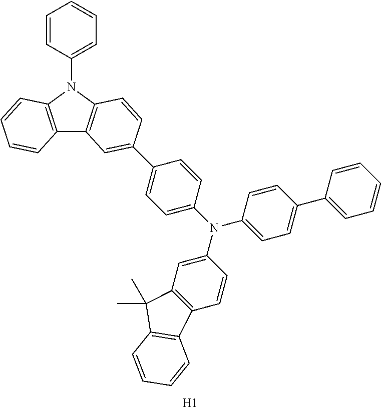

As seen from the results of Table 9, it was identified that the organic electroluminescent device using the hole transfer layer material of the blue organic electroluminescent device of the present disclosure had lower driving voltage and improved light emission efficiency compared to Comparative Example 3-1 and Comparative Example 3-2.

1. A heterocyclic compound represented by the following Chemical Formula 1: 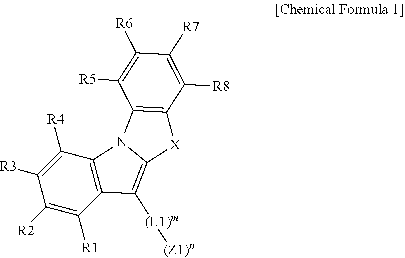
wherein, in Chemical Formula 1,
X is O; S; or NRa;
R1 to R8 and Ra are the same as or different from each other, and each independently selected from the group consisting of hydrogen; deuterium; a halogen group; —CN; a substituted or unsubstituted C1 to C60 alkyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiR11R12R13; —P(═O)R14R15; and —NR16R17, or two or more groups adjacent to each other bond to each other to form a substituted or unsubstituted C6 to C60 aromatic hydrocarbon ring or a substituted or unsubstituted C2 to C60 heteroring;
L1 is a direct bond; a substituted or unsubstituted C6 to C60 arylene group; a substituted or unsubstituted C2 to C60 heteroarylene group; or a substituted or unsubstituted divalent amine group;
Z1 is a halogen group; —CN; a substituted or unsubstituted C1 to C60 alkyl group; a substituted or unsubstituted C6 to C60 aryl group; a substituted or unsubstituted C2 to C60 heteroaryl group; —SiR11R12R13; —P(═O)R14R15; or —NR16R17;
R11 to R17 are the same as or different from each other, and each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group;
m is an integer of 0 to 4, and when m is 2 or greater, two or more L1s are the same as or different from each other; and
n is an integer of 1 to 5, and when n is 2 or greater, two or more Z1s are the same as or different from each other.

2. The heterocyclic compound of claim 1, wherein the “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, and
R, R′ and R″ are the same as or different from each other, and each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group.

3. The heterocyclic compound of claim 1, wherein Chemical Formula 1 is represented by any one of the following Chemical Formula 2 to Chemical Formula 4: 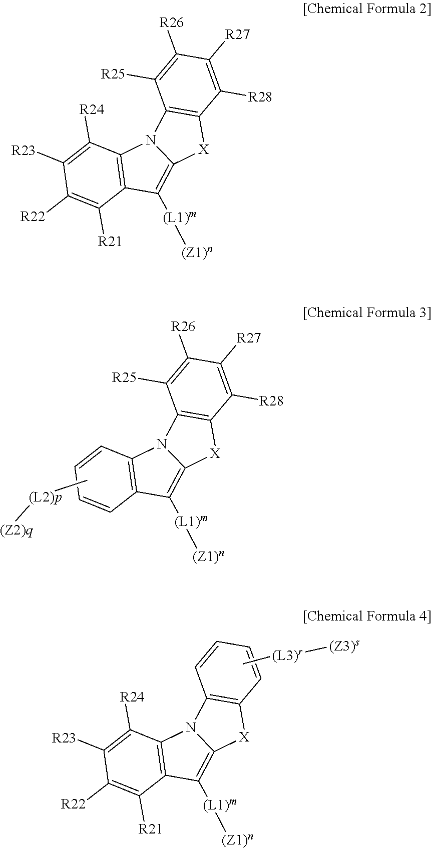
in Chemical Formulae 2 to 4,
X, m, n, L1 and Z1 have the same definitions as in Chemical Formula 1;
R21 to R28 are the same as or different from each other, and are hydrogen, or deuterium;
L2 and L3 are the same as or different from each other, and each independently a direct bond; a substituted or unsubstituted C6 to C60 arylene group; or a substituted or unsubstituted C2 to C60 heteroarylene group:
Z2 and Z3 are the same as or different from each other, and each independently a halogen group; —CN; a substituted or unsubstituted C1 to C60 alkyl group; a substituted or unsubstituted C6 to C60 aryl group; a substituted or unsubstituted C2 to C60 heteroaryl group; —SiR31R32R33; —P(═O)R34R35; or —NR36R37;
R31 to R37 are the same as or different from each other, and each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group;
p and r are an integer of 0 to 4; and
q and s are an integer of 1 to 5.

4. The heterocyclic compound of claim 1, wherein Chemical Formula 1 is represented by any one of the following Chemical Formulae 5 to 7: 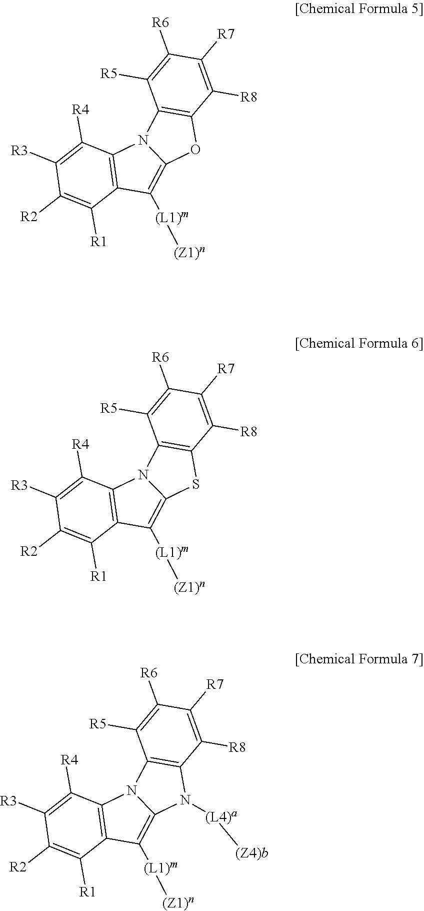
in Chemical Formulae 5 to 7,
R1 to R8, L1, Z1, m and n have the same definitions as in Chemical Formula 1;
L4 is a direct bond; a substituted or unsubstituted C6 to C60 arylene group; a substituted or unsubstituted C2 to C60 heteroarylene group; or a substituted or unsubstituted divalent amine group;
Z4 is a halogen group; —CN; a substituted or unsubstituted C1 to C60 alkyl group; a substituted or unsubstituted C6 to C60 aryl group; a substituted or unsubstituted C2 to C60 heteroaryl group; —SiR41R42R43; —P(═O)R44R45; or —NR46R47;
R41 to R47 are the same as or different from each other, and each independently hydrogen; a halogen group; —CN; a substituted or unsubstituted C1 to C60 alkyl group; a substituted or unsubstituted C6 to C60 aryl group; or a substituted or unsubstituted C2 to C60 heteroaryl group;
a is an integer of 0 to 4, and when a is 2 or greater, two or more L4s are the same as or different from each other; and
b is an integer of 1 to 5, and when b is 2 or greater, two or more Z4s are the same as or different from each other.

5. The heterocyclic compound of claim 3, wherein Chemical Formula 3 is represented by any one of the following Chemical Formula 3-1 to Chemical Formula 3-4: 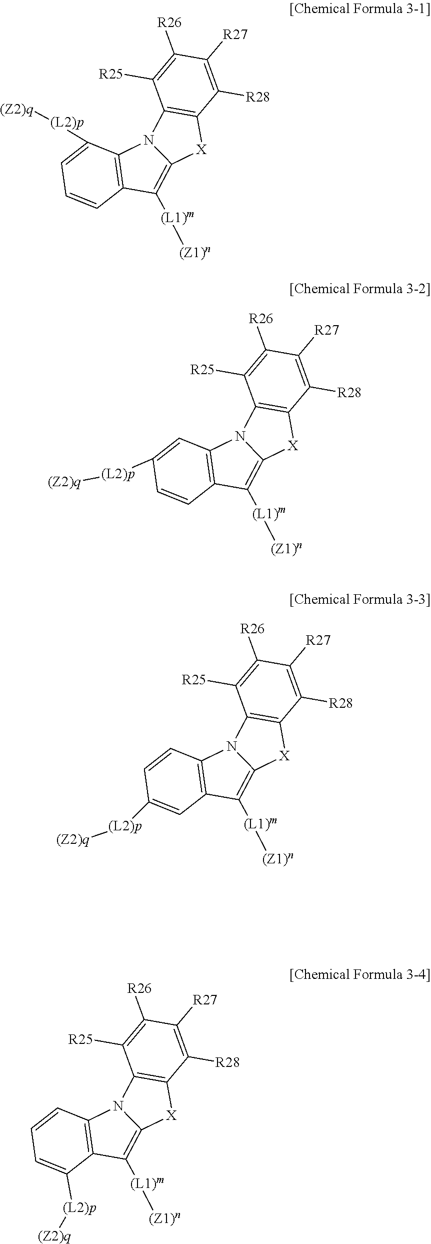
in Chemical Formulae 3-1 to 3-4,
X, L1, L2, Z1, Z2, m, n, p, q and R25 to R28 have the same definitions as in Chemical Formula 3.

6. The heterocyclic compound of claim 3, wherein Chemical Formula 4 is represented by any one of the following Chemical Formula 4-1 to Chemical Formula 4-4; 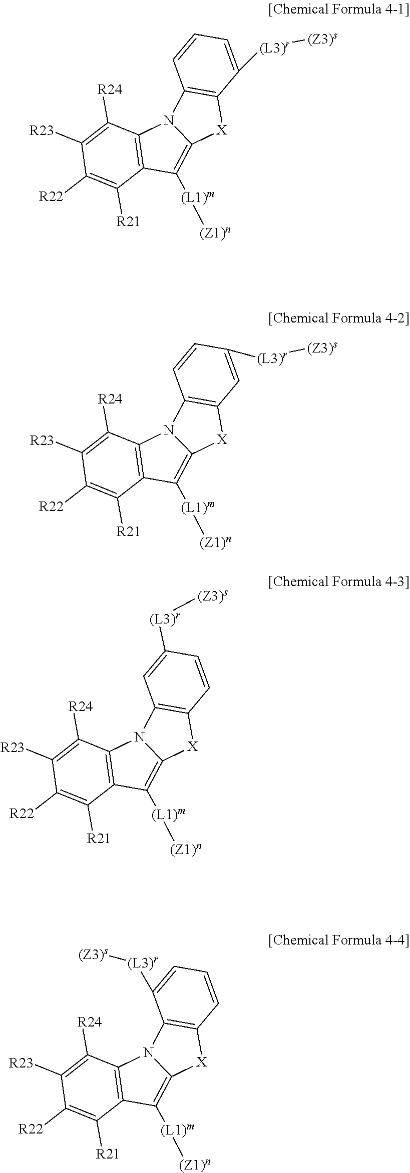
in Chemical Formulae 4-1 to 4-4,
X, L1, L3, Z1, Z3, m, n, r, s and R21 to R24 have the same definitions as in Chemical Formula 4.

7. The heterocyclic compound of claim 1, wherein Chemical Formula 1 is represented by any one of the following compounds:

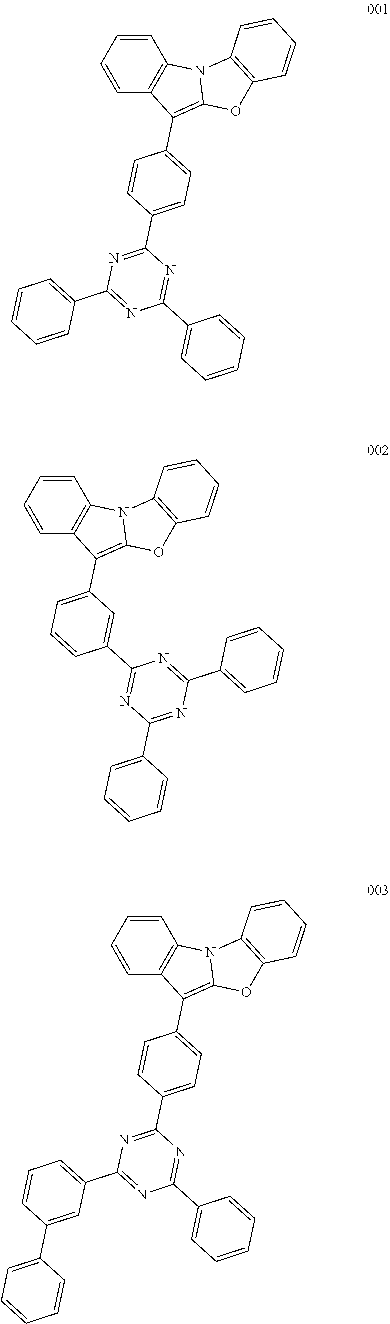 [SMILES: c1ccc(-c2nc(-c3ccccc3)nc(-c3ccc(-c4c5ccccc5n5c4oc4ccccc45)cc3)n2)cc1 ; c1ccc(-c2nc(-c3ccccc3)nc(-c3cccc(-c4c5ccccc5n5c4oc4ccccc45)c3)n2)cc1 ; c1ccc(-c2cccc(-c3nc(-c4ccccc4)nc(-c4ccc(-c5c6ccccc6n6c5oc5ccccc56)cc4)n3)c2)cc1]

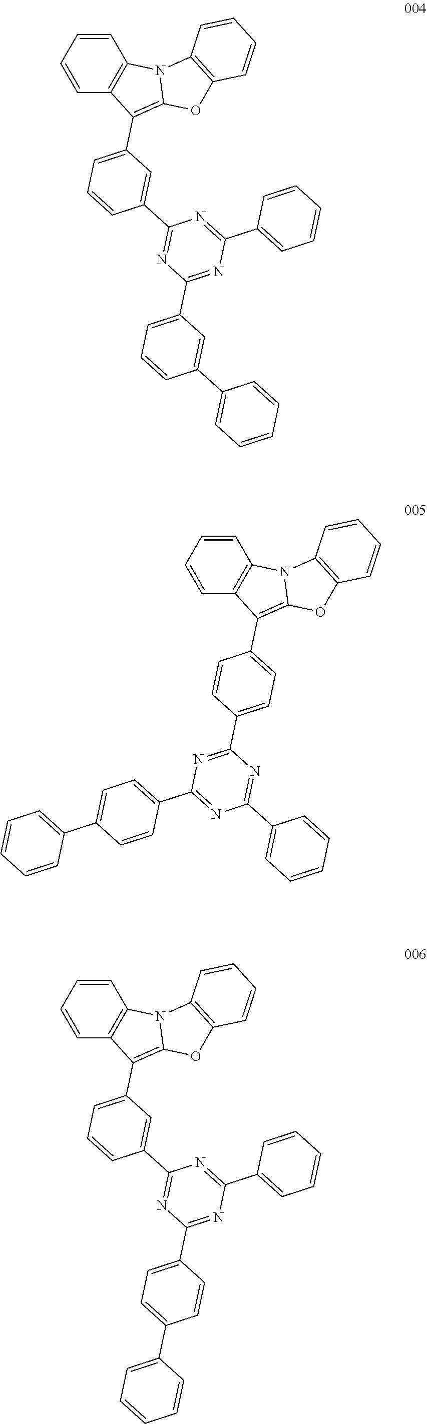 [SMILES: c1ccc(-c2cccc(-c3nc(-c4ccccc4)nc(-c4cccc(-c5c6ccccc6n6c5oc5ccccc56)c4)n3)c2)cc1 ; c1ccc(-c2ccc(-c3nc(-c4ccccc4)nc(-c4ccc(-c5c6ccccc6n6c5oc5ccccc56)cc4)n3)cc2)cc1 ; c1ccc(-c2ccc(-c3nc(-c4ccccc4)nc(-c4cccc(-c5c6ccccc6n6c5oc5ccccc56)c4)n3)cc2)cc1]

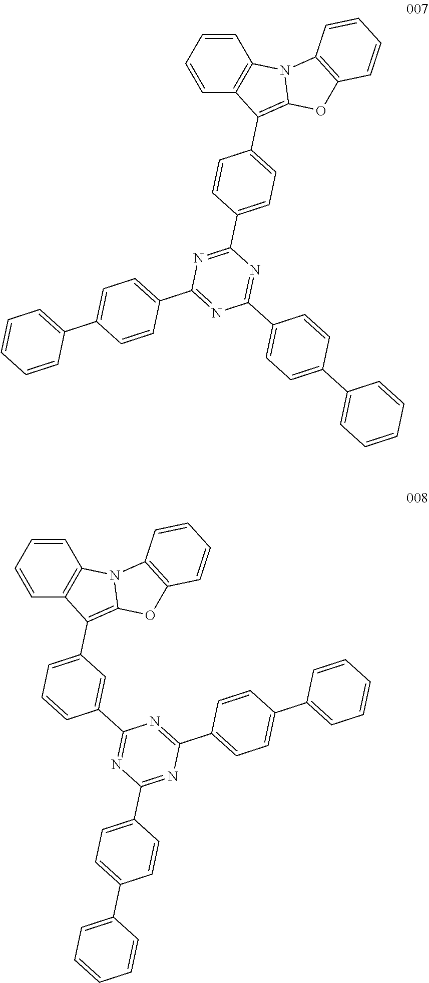 [SMILES: c1ccc(-c2ccc(-c3nc(-c4ccc(-c5ccccc5)cc4)nc(-c4ccc(-c5c6ccccc6n6c5oc5ccccc56)cc4)n3)cc2)cc1 ; c1ccc(-c2ccc(-c3nc(-c4ccc(-c5ccccc5)cc4)nc(-c4cccc(-c5c6ccccc6n6c5oc5ccccc56)c4)n3)cc2)cc1]

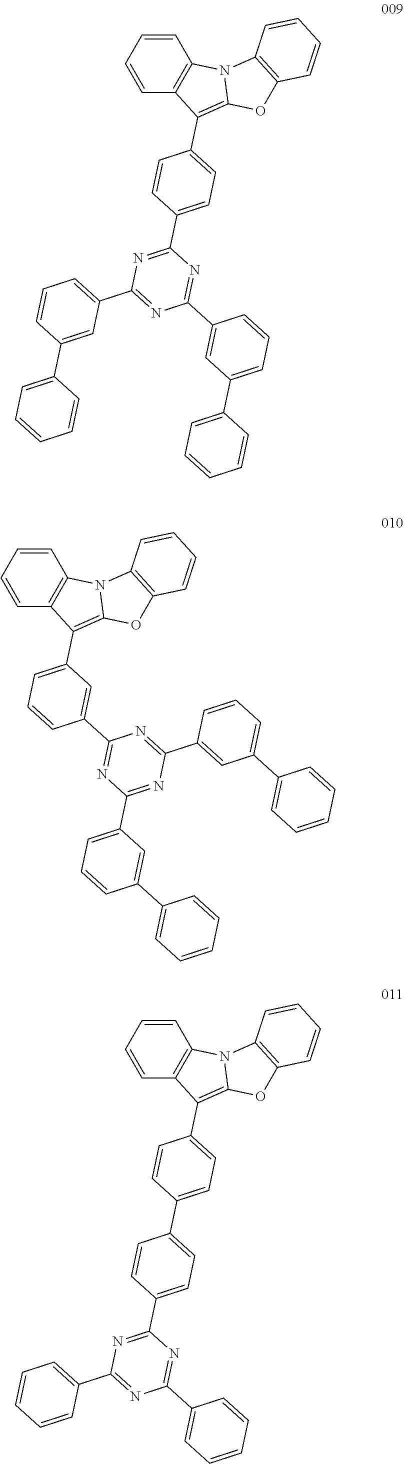 [SMILES: c1ccc(-c2cccc(-c3nc(-c4ccc(-c5c6ccccc6n6c5oc5ccccc56)cc4)nc(-c4cccc(-c5ccccc5)c4)n3)c2)cc1 ; c1ccc(-c2cccc(-c3nc(-c4cccc(-c5ccccc5)c4)nc(-c4cccc(-c5c6ccccc6n6c5oc5ccccc56)c4)n3)c2)cc1 ; c1ccc(-c2nc(-c3ccccc3)nc(-c3ccc(-c4ccc(-c5c6ccccc6n6c5oc5ccccc56)cc4)cc3)n2)cc1]

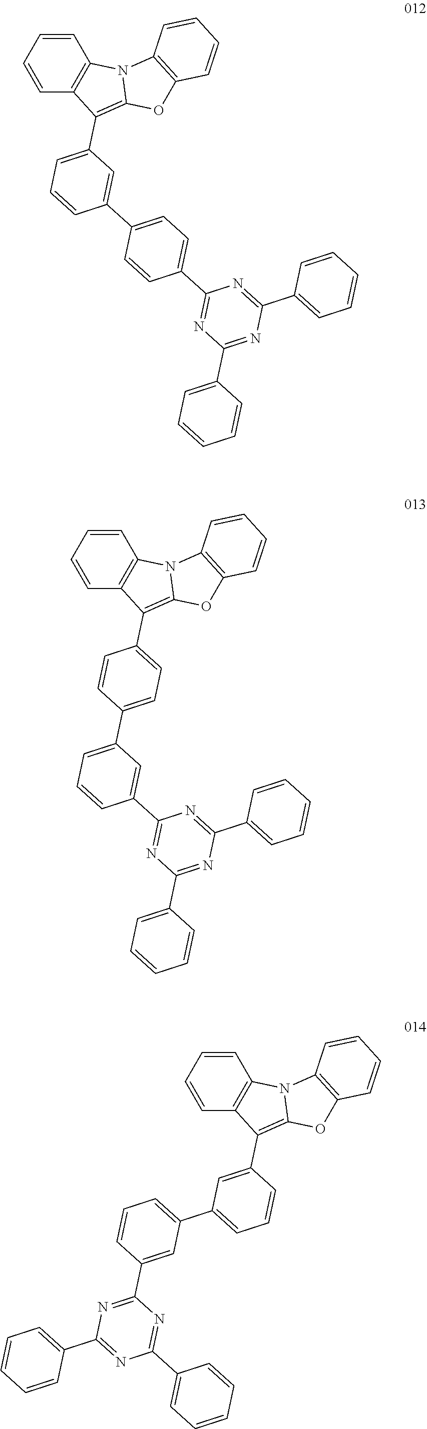 [SMILES: c1ccc(-c2nc(-c3ccccc3)nc(-c3ccc(-c4cccc(-c5c6ccccc6n6c5oc5ccccc56)c4)cc3)n2)cc1 ; c1ccc(-c2nc(-c3ccccc3)nc(-c3cccc(-c4ccc(-c5c6ccccc6n6c5oc5ccccc56)cc4)c3)n2)cc1 ; c1ccc(-c2nc(-c3ccccc3)nc(-c3cccc(-c4cccc(-c5c6ccccc6n6c5oc5ccccc56)c4)c3)n2)cc1]

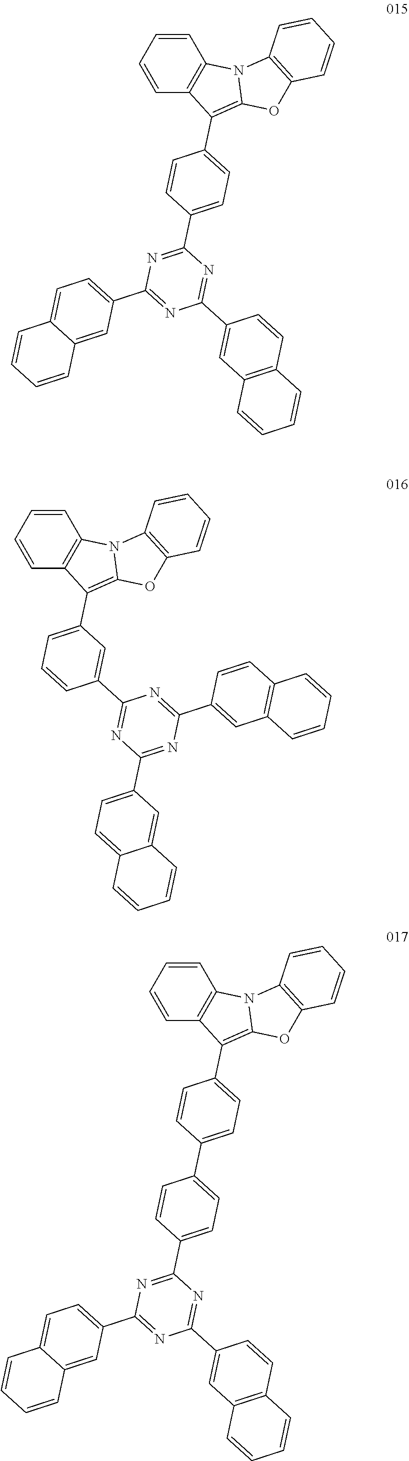 [SMILES: c1ccc2cc(-c3nc(-c4ccc(-c5c6ccccc6n6c5oc5ccccc56)cc4)nc(-c4ccc5ccccc5c4)n3)ccc2c1 ; c1cc(-c2nc(-c3ccc4ccccc4c3)nc(-c3ccc4ccccc4c3)n2)cc(-c2c3ccccc3n3c2oc2ccccc23)c1 ; c1ccc2cc(-c3nc(-c4ccc(-c5ccc(-c6c7ccccc7n7c6oc6ccccc67)cc5)cc4)nc(-c4ccc5ccccc5c4)n3)ccc2c1]

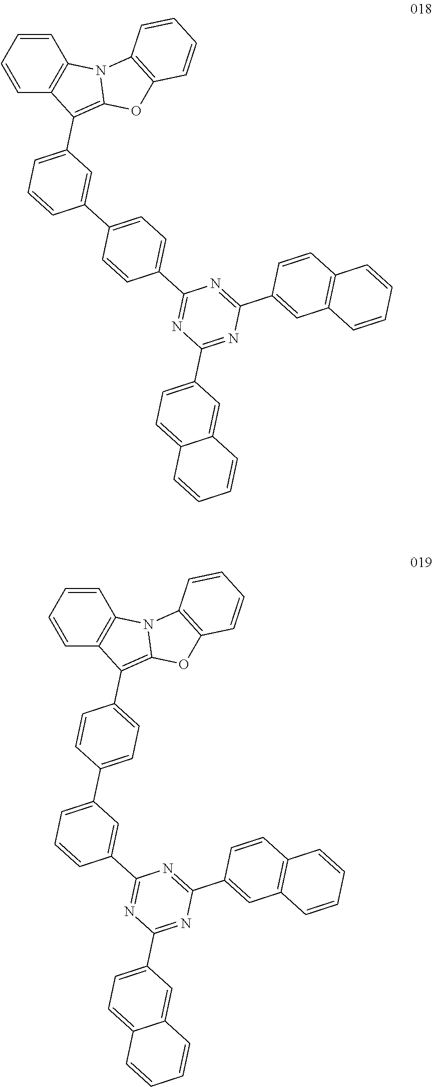 [SMILES: c1cc(-c2ccc(-c3nc(-c4ccc5ccccc5c4)nc(-c4ccc5ccccc5c4)n3)cc2)cc(-c2c3ccccc3n3c2oc2ccccc23)c1 ; c1cc(-c2ccc(-c3c4ccccc4n4c3oc3ccccc34)cc2)cc(-c2nc(-c3ccc4ccccc4c3)nc(-c3ccc4ccccc4c3)n2)c1]

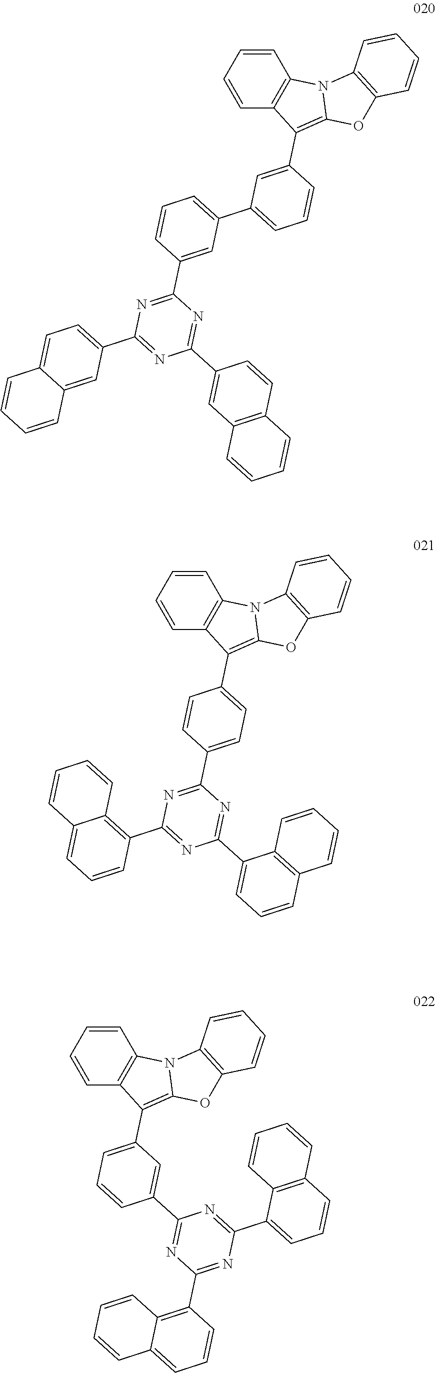 [SMILES: c1cc(-c2cccc(-c3c4ccccc4n4c3oc3ccccc34)c2)cc(-c2nc(-c3ccc4ccccc4c3)nc(-c3ccc4ccccc4c3)n2)c1 ; c1ccc2c(-c3nc(-c4ccc(-c5c6ccccc6n6c5oc5ccccc56)cc4)nc(-c4cccc5ccccc45)n3)cccc2c1 ; c1cc(-c2nc(-c3cccc4ccccc34)nc(-c3cccc4ccccc34)n2)cc(-c2c3ccccc3n3c2oc2ccccc23)c1]

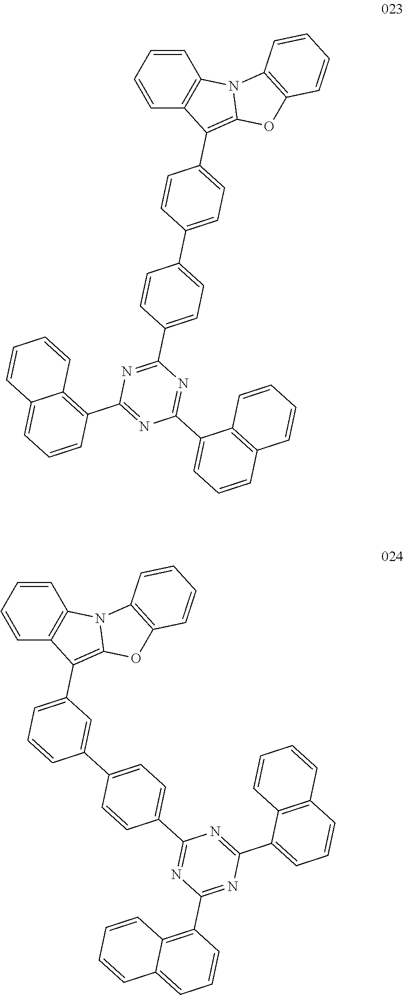 [SMILES: c1ccc2c(-c3nc(-c4ccc(-c5ccc(-c6c7ccccc7n7c6oc6ccccc67)cc5)cc4)nc(-c4cccc5ccccc45)n3)cccc2c1 ; c1cc(-c2ccc(-c3nc(-c4cccc5ccccc45)nc(-c4cccc5ccccc45)n3)cc2)cc(-c2c3ccccc3n3c2oc2ccccc23)c1]

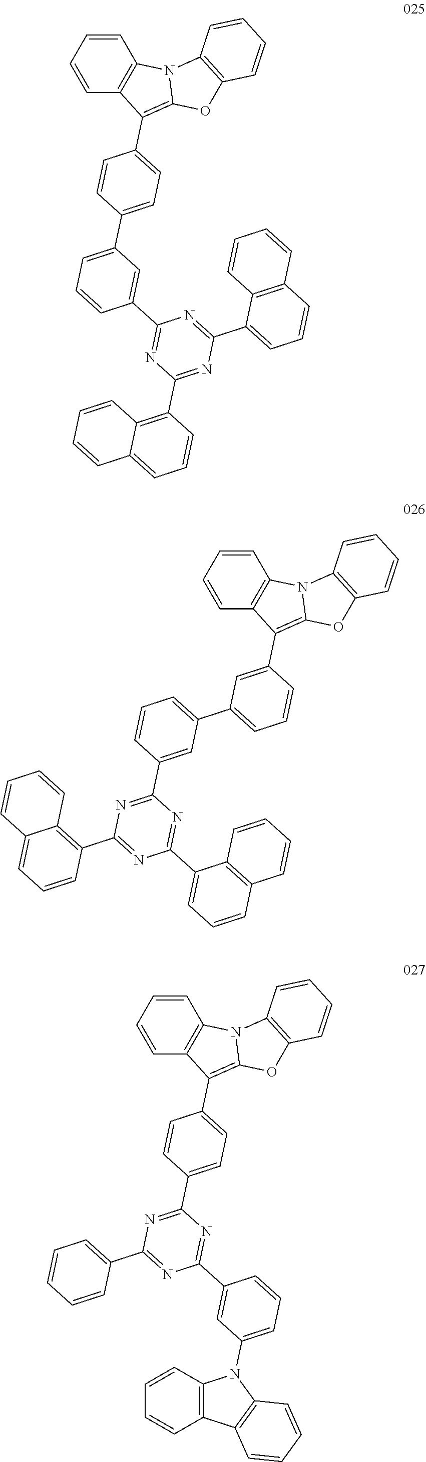 [SMILES: c1cc(-c2ccc(-c3c4ccccc4n4c3oc3ccccc34)cc2)cc(-c2nc(-c3cccc4ccccc34)nc(-c3cccc4ccccc34)n2)c1 ; c1cc(-c2cccc(-c3c4ccccc4n4c3oc3ccccc34)c2)cc(-c2nc(-c3cccc4ccccc34)nc(-c3cccc4ccccc34)n2)c1 ; c1ccc(-c2nc(-c3ccc(-c4c5ccccc5n5c4oc4ccccc45)cc3)nc(-c3cccc(-n4c5ccccc5c5ccccc54)c3)n2)cc1]

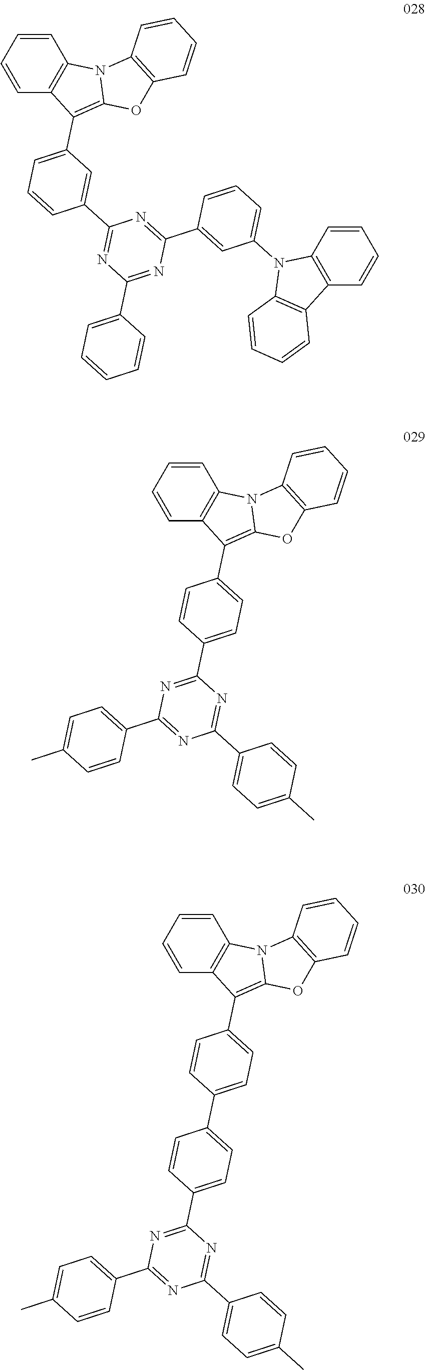 [SMILES: c1ccc(-c2nc(-c3cccc(-c4c5ccccc5n5c4oc4ccccc45)c3)nc(-c3cccc(-n4c5ccccc5c5ccccc54)c3)n2)cc1 ; Cc1ccc(-c2nc(-c3ccc(C)cc3)nc(-c3ccc(-c4c5ccccc5n5c4oc4ccccc45)cc3)n2)cc1 ; Cc1ccc(-c2nc(-c3ccc(C)cc3)nc(-c3ccc(-c4ccc(-c5c6ccccc6n6c5oc5ccccc56)cc4)cc3)n2)cc1]

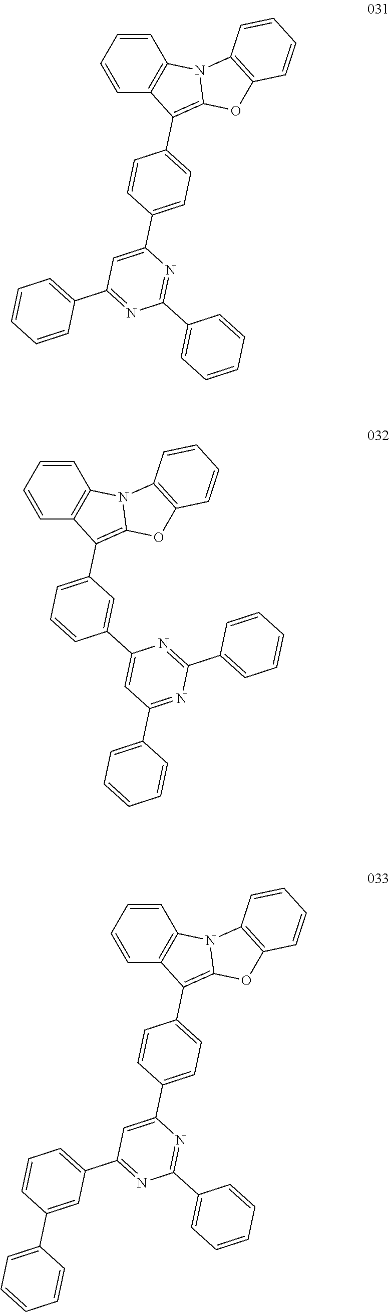 [SMILES: c1ccc(-c2cc(-c3ccc(-c4c5ccccc5n5c4oc4ccccc45)cc3)nc(-c3ccccc3)n2)cc1 ; c1ccc(-c2cc(-c3cccc(-c4c5ccccc5n5c4oc4ccccc45)c3)nc(-c3ccccc3)n2)cc1 ; c1ccc(-c2cccc(-c3cc(-c4ccc(-c5c6ccccc6n6c5oc5ccccc56)cc4)nc(-c4ccccc4)n3)c2)cc1]

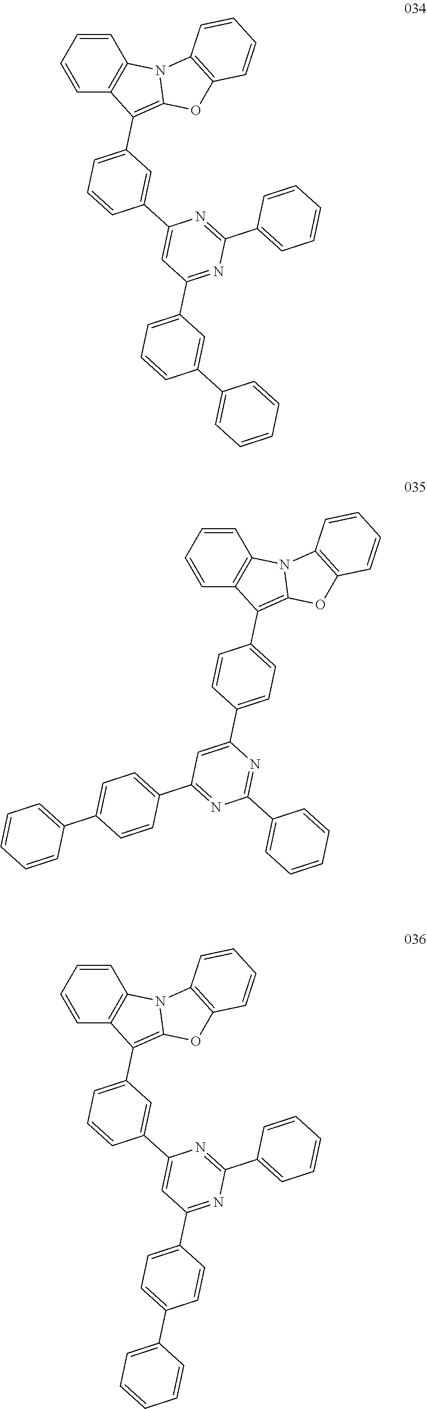 [SMILES: c1ccc(-c2cccc(-c3cc(-c4cccc(-c5c6ccccc6n6c5oc5ccccc56)c4)nc(-c4ccccc4)n3)c2)cc1 ; c1ccc(-c2ccc(-c3cc(-c4ccc(-c5c6ccccc6n6c5oc5ccccc56)cc4)nc(-c4ccccc4)n3)cc2)cc1 ; c1ccc(-c2ccc(-c3cc(-c4cccc(-c5c6ccccc6n6c5oc5ccccc56)c4)nc(-c4ccccc4)n3)cc2)cc1]

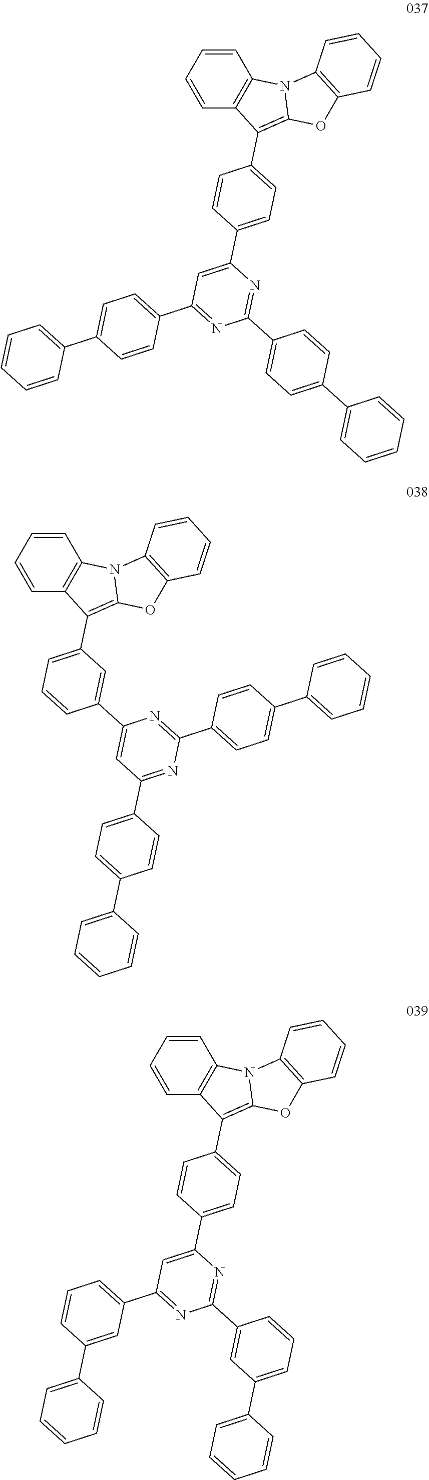 [SMILES: c1ccc(-c2ccc(-c3cc(-c4ccc(-c5c6ccccc6n6c5oc5ccccc56)cc4)nc(-c4ccc(-c5ccccc5)cc4)n3)cc2)cc1 ; c1ccc(-c2ccc(-c3cc(-c4cccc(-c5c6ccccc6n6c5oc5ccccc56)c4)nc(-c4ccc(-c5ccccc5)cc4)n3)cc2)cc1 ; c1ccc(-c2cccc(-c3cc(-c4ccc(-c5c6ccccc6n6c5oc5ccccc56)cc4)nc(-c4cccc(-c5ccccc5)c4)n3)c2)cc1]

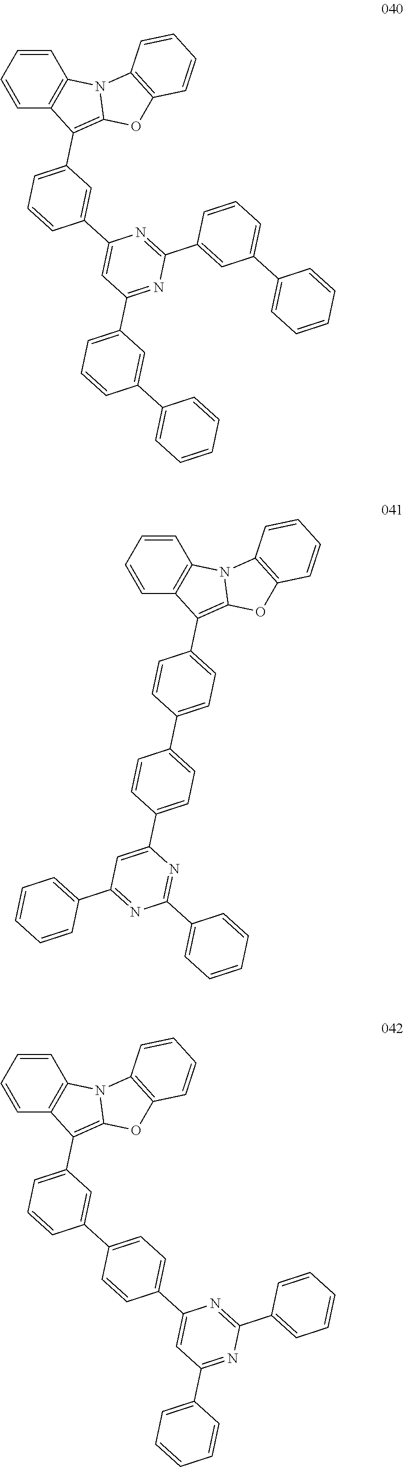 [SMILES: c1ccc(-c2cccc(-c3cc(-c4cccc(-c5c6ccccc6n6c5oc5ccccc56)c4)nc(-c4cccc(-c5ccccc5)c4)n3)c2)cc1 ; c1ccc(-c2cc(-c3ccc(-c4ccc(-c5c6ccccc6n6c5oc5ccccc56)cc4)cc3)nc(-c3ccccc3)n2)cc1 ; c1ccc(-c2cc(-c3ccc(-c4cccc(-c5c6ccccc6n6c5oc5ccccc56)c4)cc3)nc(-c3ccccc3)n2)cc1]

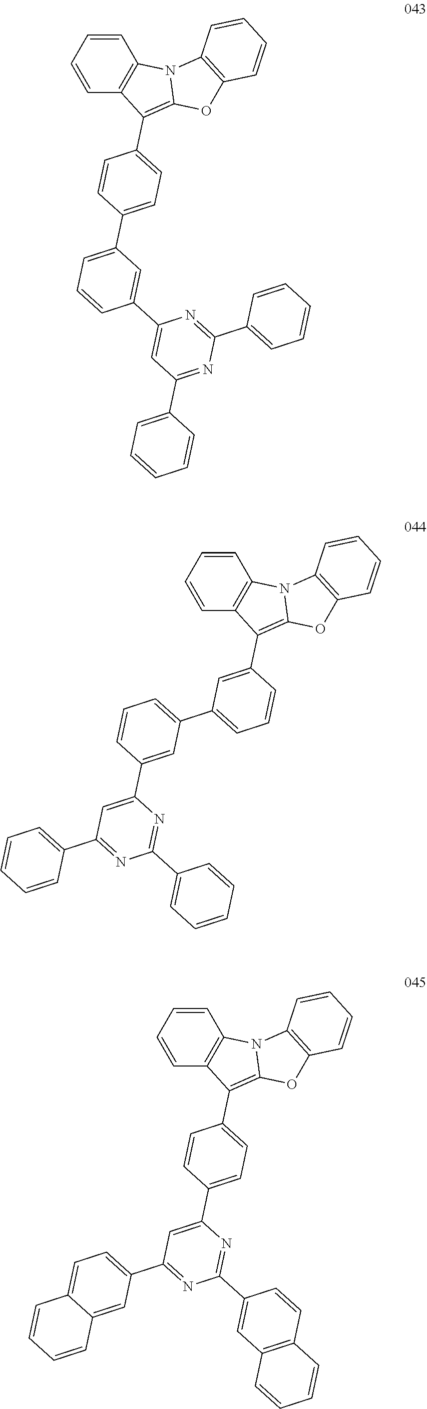 [SMILES: c1ccc(-c2cc(-c3cccc(-c4ccc(-c5c6ccccc6n6c5oc5ccccc56)cc4)c3)nc(-c3ccccc3)n2)cc1 ; c1ccc(-c2cc(-c3cccc(-c4cccc(-c5c6ccccc6n6c5oc5ccccc56)c4)c3)nc(-c3ccccc3)n2)cc1 ; c1ccc2cc(-c3cc(-c4ccc(-c5c6ccccc6n6c5oc5ccccc56)cc4)nc(-c4ccc5ccccc5c4)n3)ccc2c1]

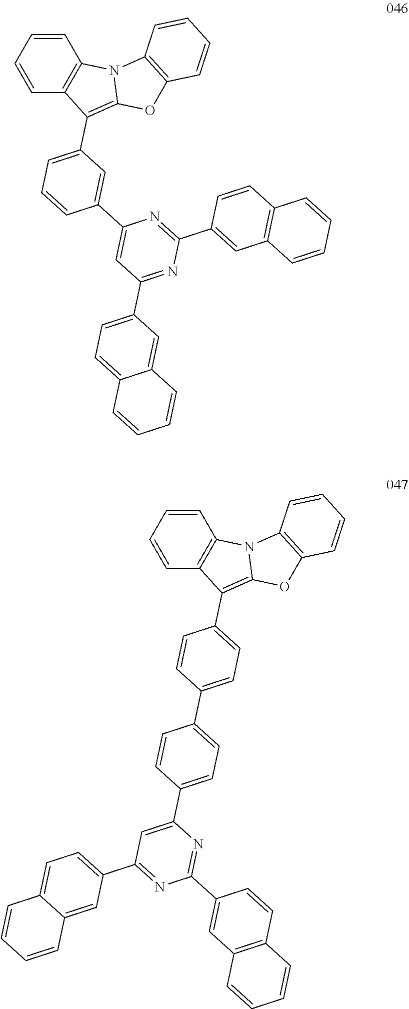 [SMILES: c1cc(-c2cc(-c3ccc4ccccc4c3)nc(-c3ccc4ccccc4c3)n2)cc(-c2c3ccccc3n3c2oc2ccccc23)c1 ; c1ccc2cc(-c3cc(-c4ccc(-c5ccc(-c6c7ccccc7n7c6oc6ccccc67)cc5)cc4)nc(-c4ccc5ccccc5c4)n3)ccc2c1]

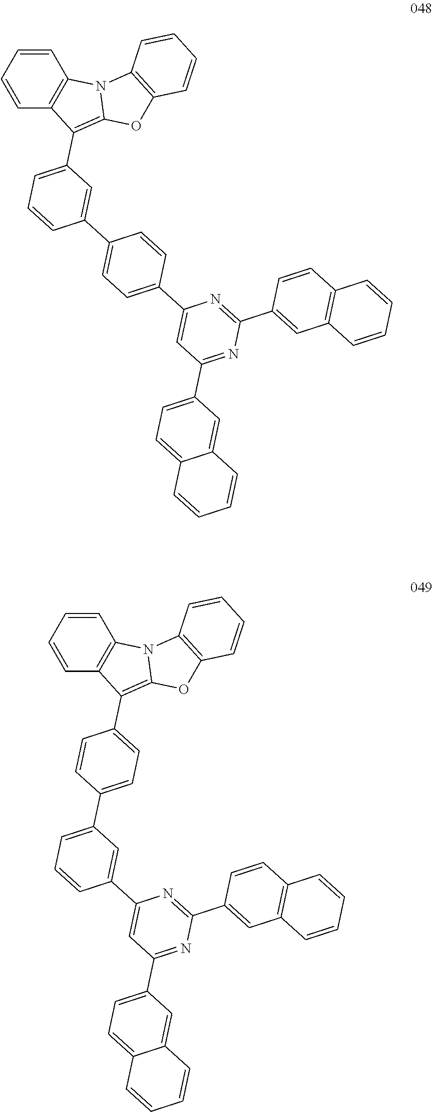 [SMILES: c1cc(-c2ccc(-c3cc(-c4ccc5ccccc5c4)nc(-c4ccc5ccccc5c4)n3)cc2)cc(-c2c3ccccc3n3c2oc2ccccc23)c1 ; c1cc(-c2ccc(-c3c4ccccc4n4c3oc3ccccc34)cc2)cc(-c2cc(-c3ccc4ccccc4c3)nc(-c3ccc4ccccc4c3)n2)c1]

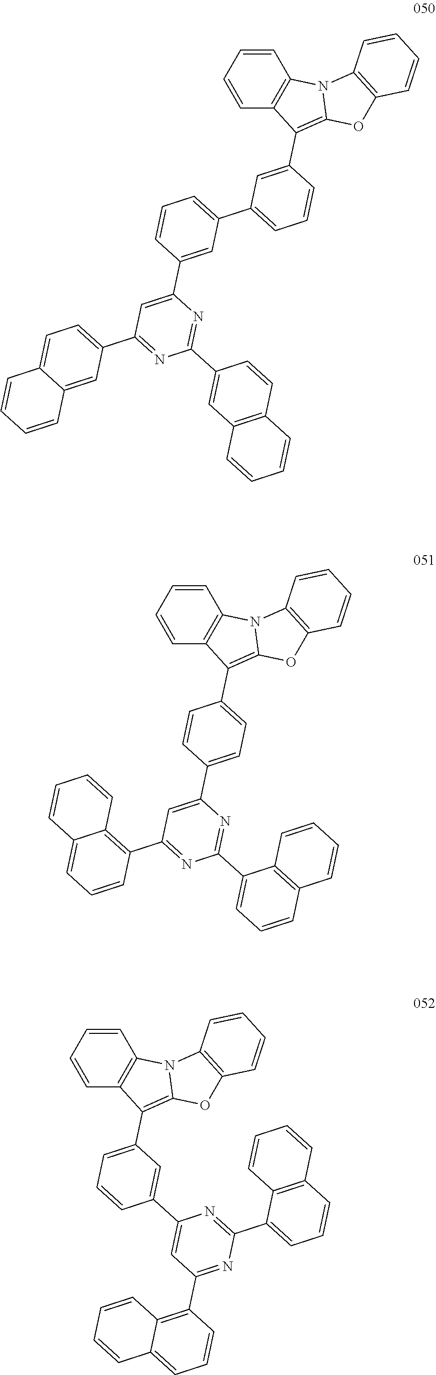 [SMILES: c1cc(-c2cccc(-c3c4ccccc4n4c3oc3ccccc34)c2)cc(-c2cc(-c3ccc4ccccc4c3)nc(-c3ccc4ccccc4c3)n2)c1 ; c1ccc2c(-c3cc(-c4ccc(-c5c6ccccc6n6c5oc5ccccc56)cc4)nc(-c4cccc5ccccc45)n3)cccc2c1 ; c1cc(-c2cc(-c3cccc4ccccc34)nc(-c3cccc4ccccc34)n2)cc(-c2c3ccccc3n3c2oc2ccccc23)c1]

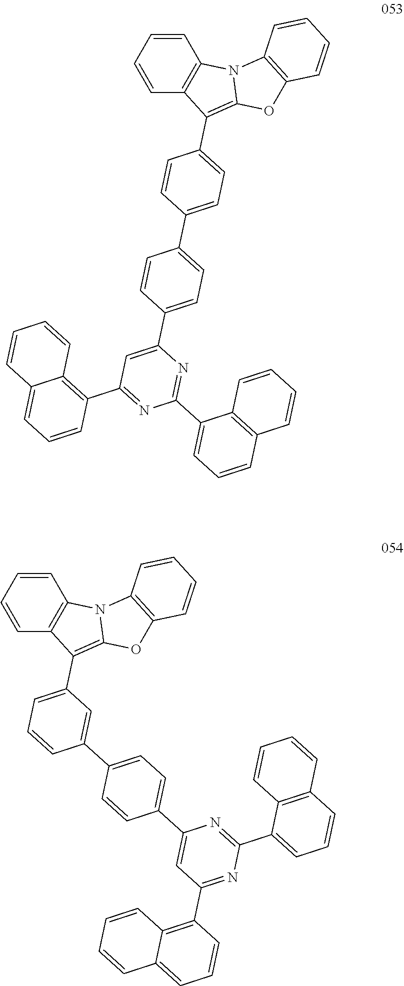 [SMILES: c1ccc2c(-c3cc(-c4ccc(-c5ccc(-c6c7ccccc7n7c6oc6ccccc67)cc5)cc4)nc(-c4cccc5ccccc45)n3)cccc2c1 ; c1cc(-c2ccc(-c3cc(-c4cccc5ccccc45)nc(-c4cccc5ccccc45)n3)cc2)cc(-c2c3ccccc3n3c2oc2ccccc23)c1]

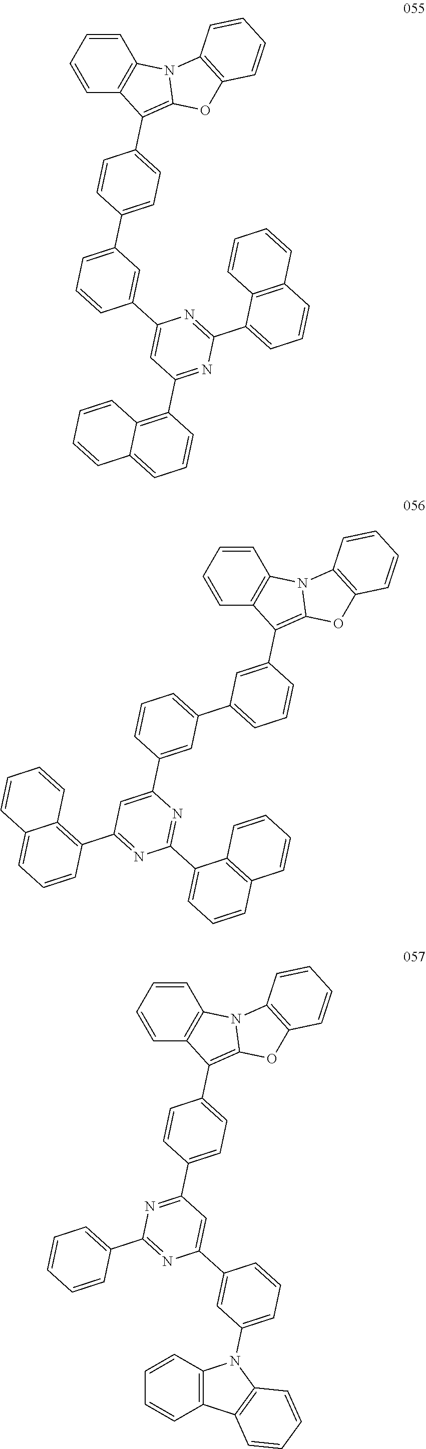 [SMILES: c1cc(-c2ccc(-c3c4ccccc4n4c3oc3ccccc34)cc2)cc(-c2cc(-c3cccc4ccccc34)nc(-c3cccc4ccccc34)n2)c1 ; c1cc(-c2cccc(-c3c4ccccc4n4c3oc3ccccc34)c2)cc(-c2cc(-c3cccc4ccccc34)nc(-c3cccc4ccccc34)n2)c1 ; c1ccc(-c2nc(-c3ccc(-c4c5ccccc5n5c4oc4ccccc45)cc3)cc(-c3cccc(-n4c5ccccc5c5ccccc54)c3)n2)cc1]

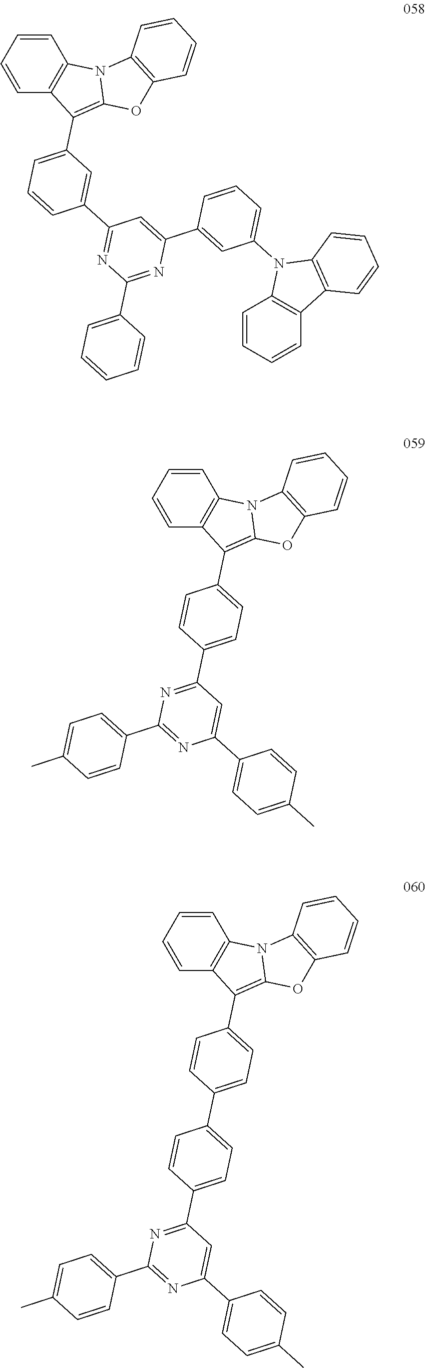 [SMILES: c1ccc(-c2nc(-c3cccc(-c4c5ccccc5n5c4oc4ccccc45)c3)cc(-c3cccc(-n4c5ccccc5c5ccccc54)c3)n2)cc1 ; Cc1ccc(-c2cc(-c3ccc(-c4c5ccccc5n5c4oc4ccccc45)cc3)nc(-c3ccc(C)cc3)n2)cc1 ; Cc1ccc(-c2cc(-c3ccc(-c4ccc(-c5c6ccccc6n6c5oc5ccccc56)cc4)cc3)nc(-c3ccc(C)cc3)n2)cc1]

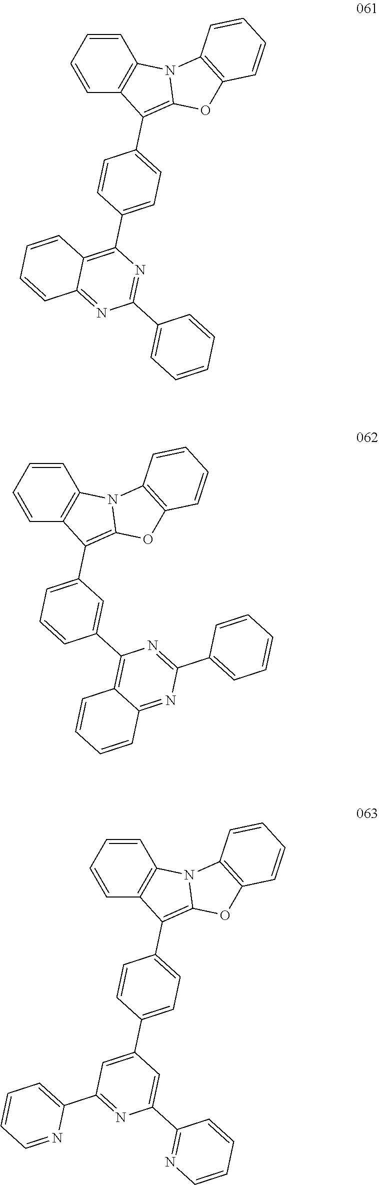 [SMILES: c1ccc(-c2nc(-c3ccc(-c4c5ccccc5n5c4oc4ccccc45)cc3)c3ccccc3n2)cc1 ; c1ccc(-c2nc(-c3cccc(-c4c5ccccc5n5c4oc4ccccc45)c3)c3ccccc3n2)cc1 ; c1ccc(-c2cc(-c3ccc(-c4c5ccccc5n5c4oc4ccccc45)cc3)cc(-c3ccccn3)n2)nc1]

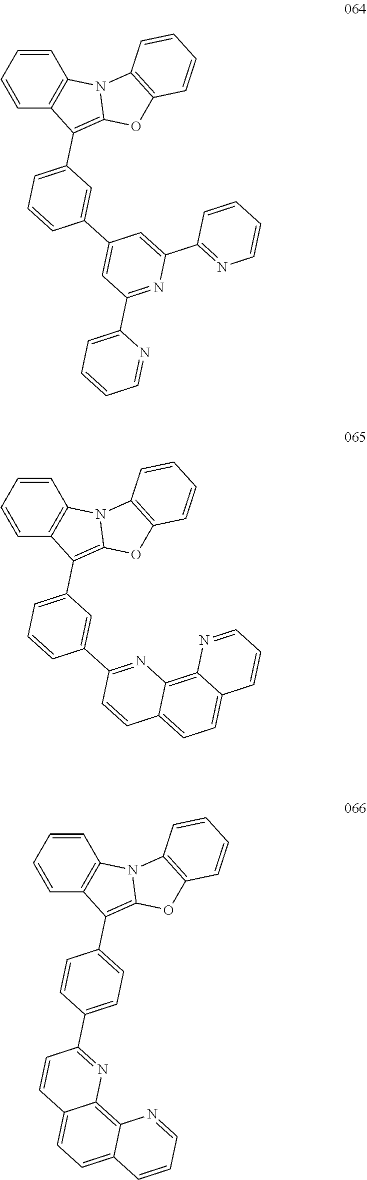 [SMILES: c1ccc(-c2cc(-c3cccc(-c4c5ccccc5n5c4oc4ccccc45)c3)cc(-c3ccccn3)n2)nc1 ; c1cc(-c2ccc3ccc4cccnc4c3n2)cc(-c2c3ccccc3n3c2oc2ccccc23)c1 ; c1cnc2c(c1)ccc1ccc(-c3ccc(-c4c5ccccc5n5c4oc4ccccc45)cc3)nc12]

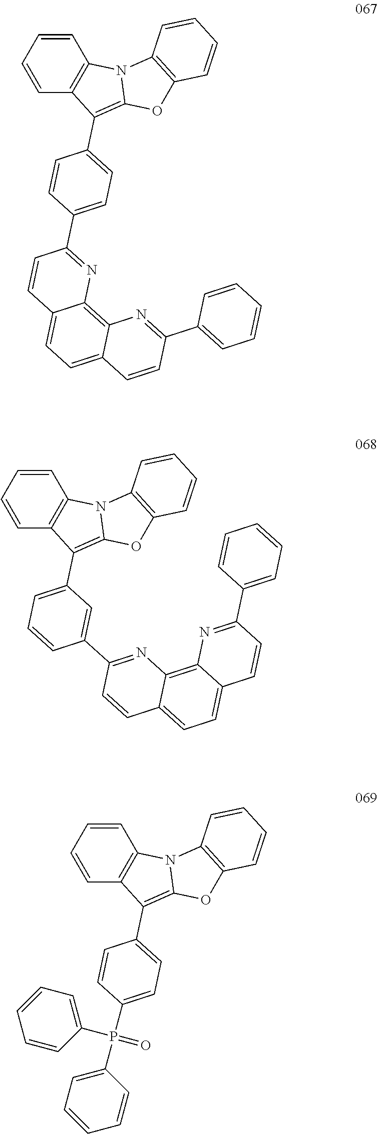 [SMILES: c1ccc(-c2ccc3ccc4ccc(-c5ccc(-c6c7ccccc7n7c6oc6ccccc67)cc5)nc4c3n2)cc1 ; c1ccc(-c2ccc3ccc4ccc(-c5cccc(-c6c7ccccc7n7c6oc6ccccc67)c5)nc4c3n2)cc1 ; O=P(c1ccccc1)(c1ccccc1)c1ccc(-c2c3ccccc3n3c2oc2ccccc23)cc1]

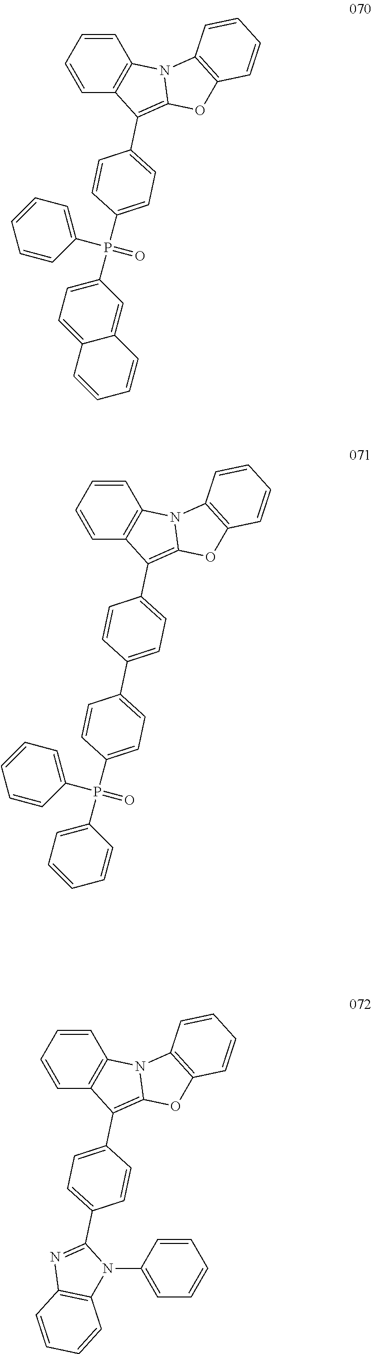 [SMILES: O=P(c1ccccc1)(c1ccc(-c2c3ccccc3n3c2oc2ccccc23)cc1)c1ccc2ccccc2c1 ; O=P(c1ccccc1)(c1ccccc1)c1ccc(-c2ccc(-c3c4ccccc4n4c3oc3ccccc34)cc2)cc1 ; c1ccc(-n2c(-c3ccc(-c4c5ccccc5n5c4oc4ccccc45)cc3)nc3ccccc32)cc1]

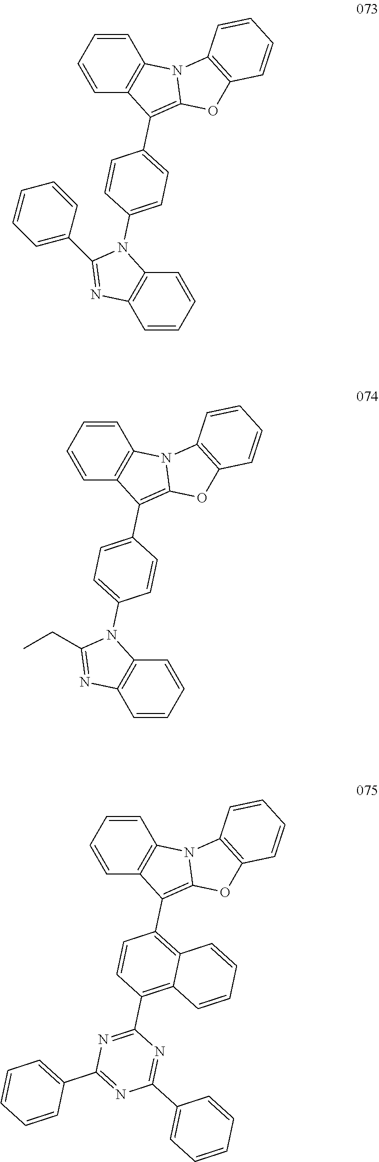 [SMILES: c1ccc(-c2nc3ccccc3n2-c2ccc(-c3c4ccccc4n4c3oc3ccccc34)cc2)cc1 ; CCc1nc2ccccc2n1-c1ccc(-c2c3ccccc3n3c2oc2ccccc23)cc1 ; c1ccc(-c2nc(-c3ccccc3)nc(-c3ccc(-c4c5ccccc5n5c4oc4ccccc45)c4ccccc34)n2)cc1]

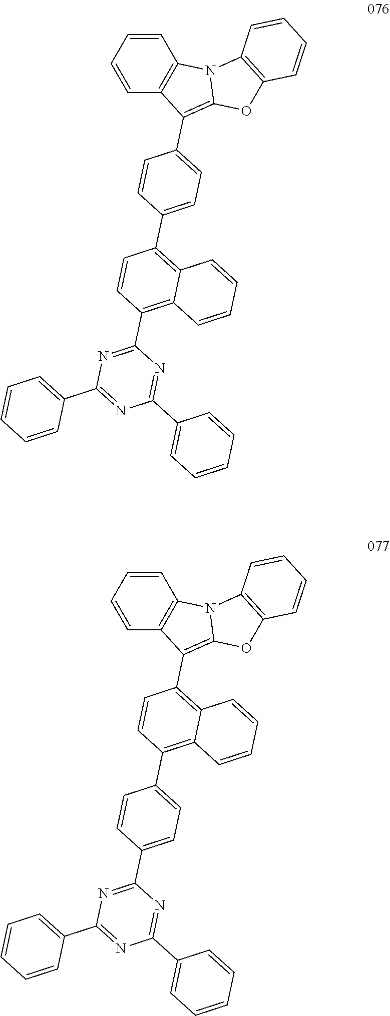 [SMILES: c1ccc(-c2nc(-c3ccccc3)nc(-c3ccc(-c4ccc(-c5c6ccccc6n6c5oc5ccccc56)cc4)c4ccccc34)n2)cc1 ; c1ccc(-c2nc(-c3ccccc3)nc(-c3ccc(-c4ccc(-c5c6ccccc6n6c5oc5ccccc56)c5ccccc45)cc3)n2)cc1]

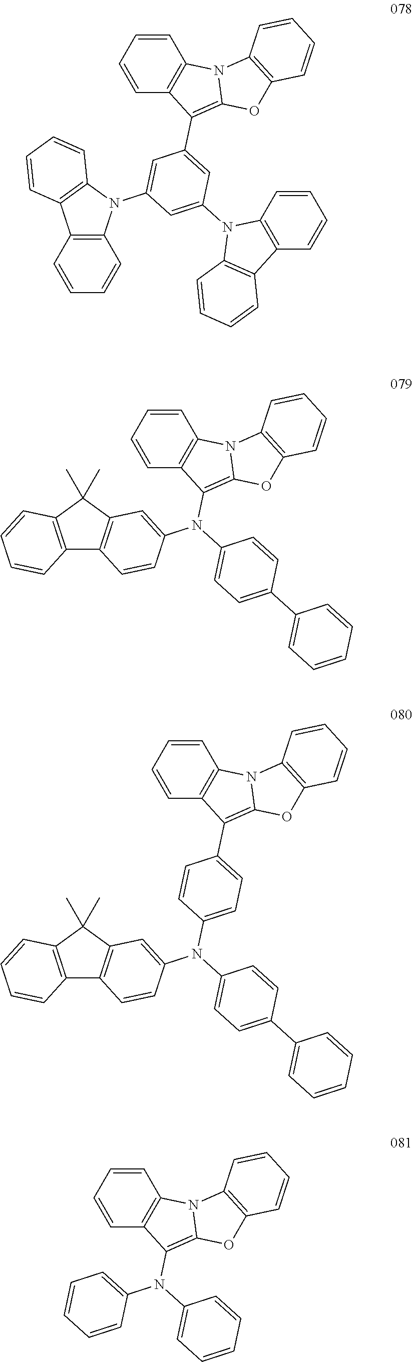 [SMILES: c1ccc2c(c1)oc1c(-c3cc(-n4c5ccccc5c5ccccc54)cc(-n4c5ccccc5c5ccccc54)c3)c3ccccc3n12 ; CC1(C)c2ccccc2-c2ccc(N(c3ccc(-c4ccccc4)cc3)c3c4ccccc4n4c3oc3ccccc34)cc21 ; CC1(C)c2ccccc2-c2ccc(N(c3ccc(-c4ccccc4)cc3)c3ccc(-c4c5ccccc5n5c4oc4ccccc45)cc3)cc21 ; c1ccc(N(c2ccccc2)c2c3ccccc3n3c2oc2ccccc23)cc1]

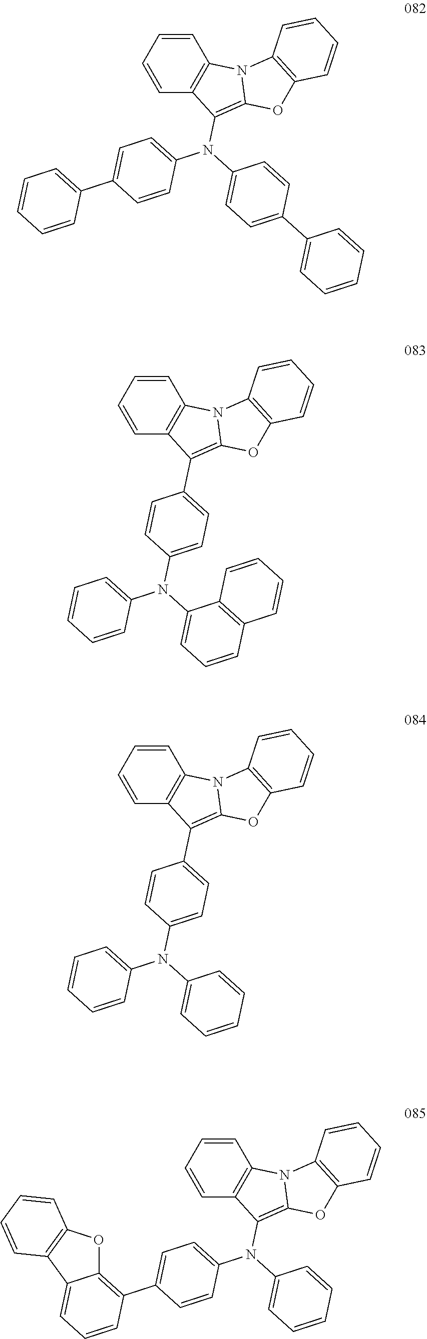 [SMILES: c1ccc(-c2ccc(N(c3ccc(-c4ccccc4)cc3)c3c4ccccc4n4c3oc3ccccc34)cc2)cc1 ; c1ccc(N(c2ccc(-c3c4ccccc4n4c3oc3ccccc34)cc2)c2cccc3ccccc23)cc1 ; c1ccc(N(c2ccccc2)c2ccc(-c3c4ccccc4n4c3oc3ccccc34)cc2)cc1 ; c1ccc(N(c2ccc(-c3cccc4c3oc3ccccc34)cc2)c2c3ccccc3n3c2oc2ccccc23)cc1]

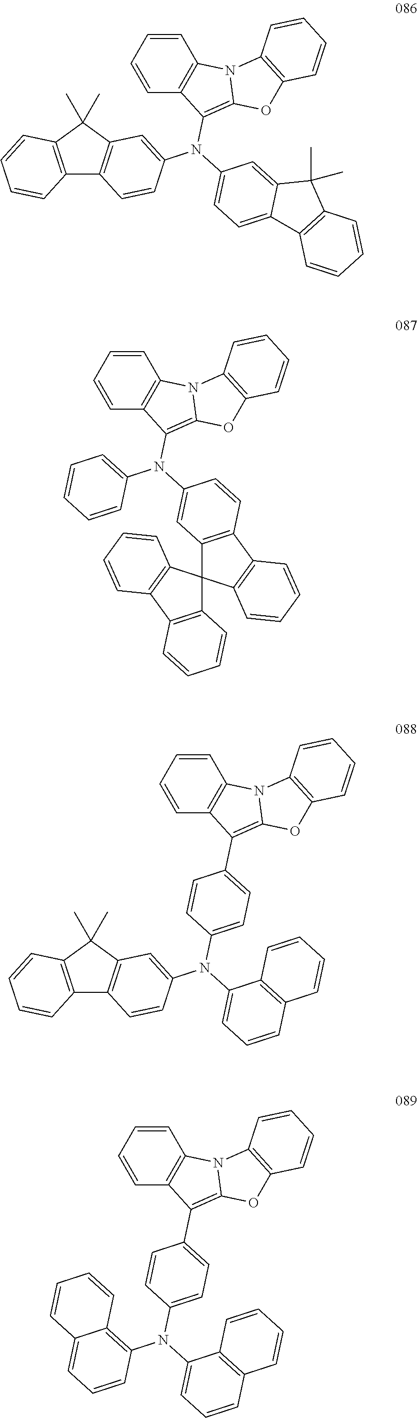 [SMILES: CC1(C)c2ccccc2-c2ccc(N(c3ccc4c(c3)C(C)(C)c3ccccc3-4)c3c4ccccc4n4c3oc3ccccc34)cc21 ; c1ccc(N(c2ccc3c(c2)C2(c4ccccc4-c4ccccc42)c2ccccc2-3)c2c3ccccc3n3c2oc2ccccc23)cc1 ; CC1(C)c2ccccc2-c2ccc(N(c3ccc(-c4c5ccccc5n5c4oc4ccccc45)cc3)c3cccc4ccccc34)cc21 ; c1ccc2c(N(c3ccc(-c4c5ccccc5n5c4oc4ccccc45)cc3)c3cccc4ccccc34)cccc2c1]

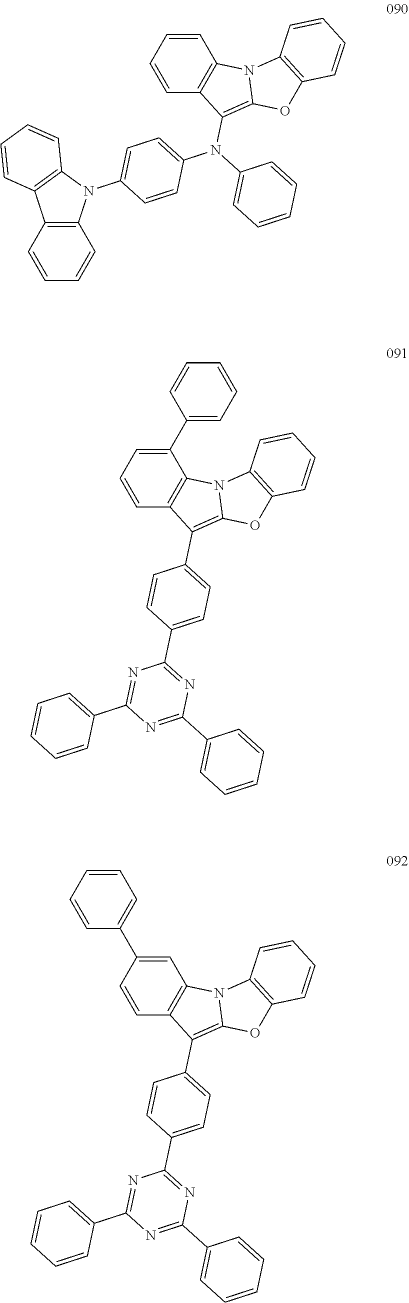 [SMILES: c1ccc(N(c2ccc(-n3c4ccccc4c4ccccc43)cc2)c2c3ccccc3n3c2oc2ccccc23)cc1 ; c1ccc(-c2nc(-c3ccccc3)nc(-c3ccc(-c4c5cccc(-c6ccccc6)c5n5c4oc4ccccc45)cc3)n2)cc1 ; c1ccc(-c2ccc3c(-c4ccc(-c5nc(-c6ccccc6)nc(-c6ccccc6)n5)cc4)c4oc5ccccc5n4c3c2)cc1]

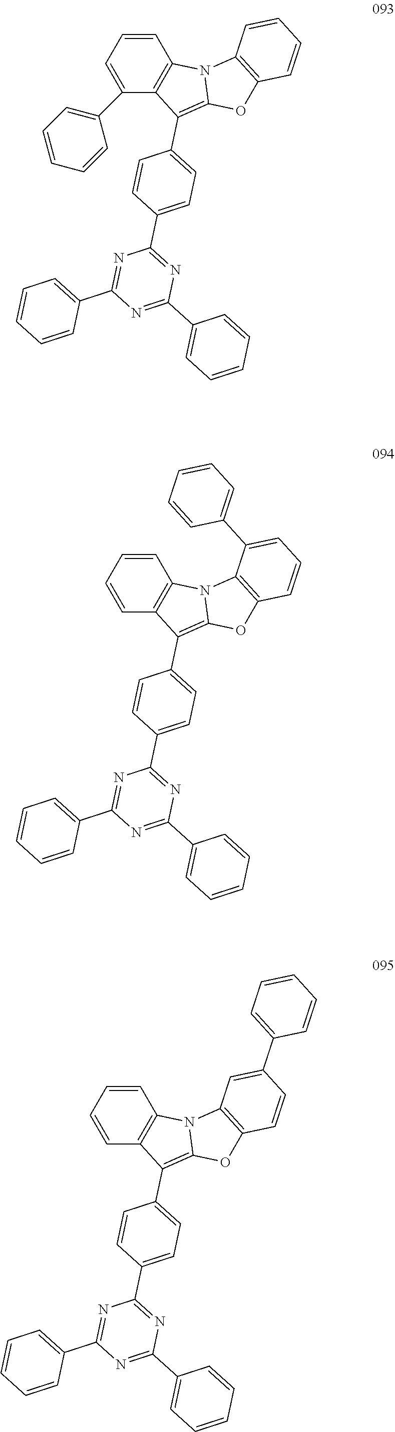 [SMILES: c1ccc(-c2nc(-c3ccccc3)nc(-c3ccc(-c4c5c(-c6ccccc6)cccc5n5c4oc4ccccc45)cc3)n2)cc1 ; c1ccc(-c2nc(-c3ccccc3)nc(-c3ccc(-c4c5ccccc5n5c4oc4cccc(-c6ccccc6)c45)cc3)n2)cc1 ; c1ccc(-c2ccc3oc4c(-c5ccc(-c6nc(-c7ccccc7)nc(-c7ccccc7)n6)cc5)c5ccccc5n4c3c2)cc1]

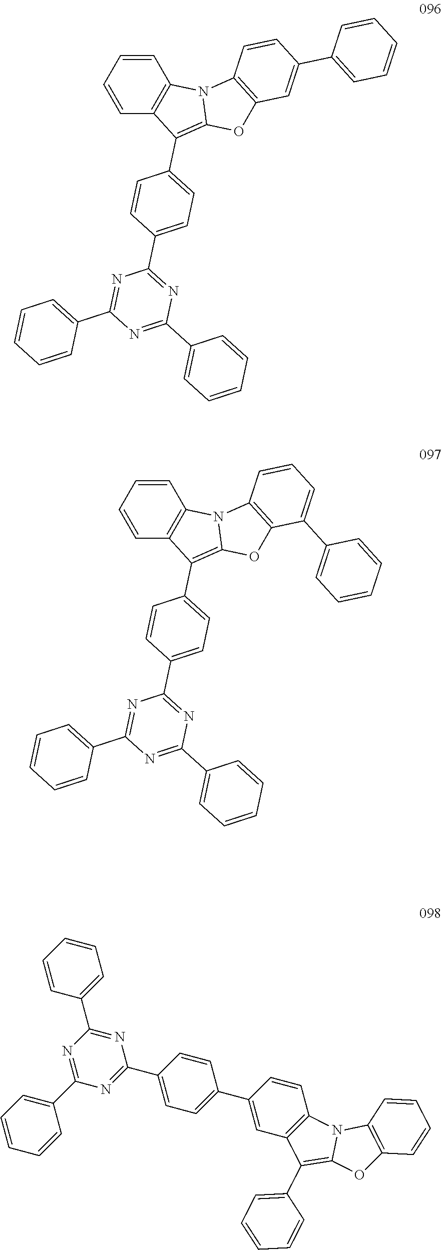 [SMILES: c1ccc(-c2ccc3c(c2)oc2c(-c4ccc(-c5nc(-c6ccccc6)nc(-c6ccccc6)n5)cc4)c4ccccc4n23)cc1 ; c1ccc(-c2nc(-c3ccccc3)nc(-c3ccc(-c4c5ccccc5n5c4oc4c(-c6ccccc6)cccc45)cc3)n2)cc1 ; c1ccc(-c2nc(-c3ccccc3)nc(-c3ccc(-c4ccc5c(c4)c(-c4ccccc4)c4oc6ccccc6n45)cc3)n2)cc1]

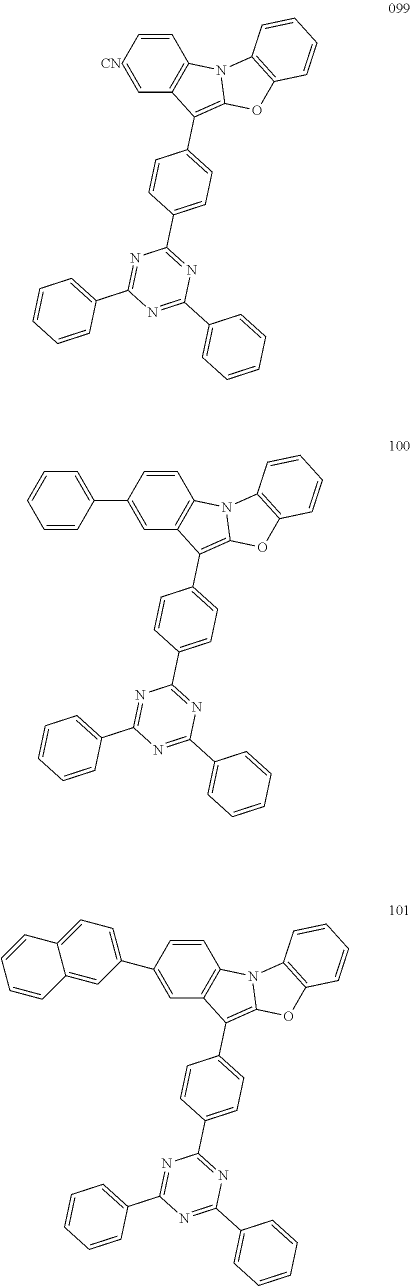

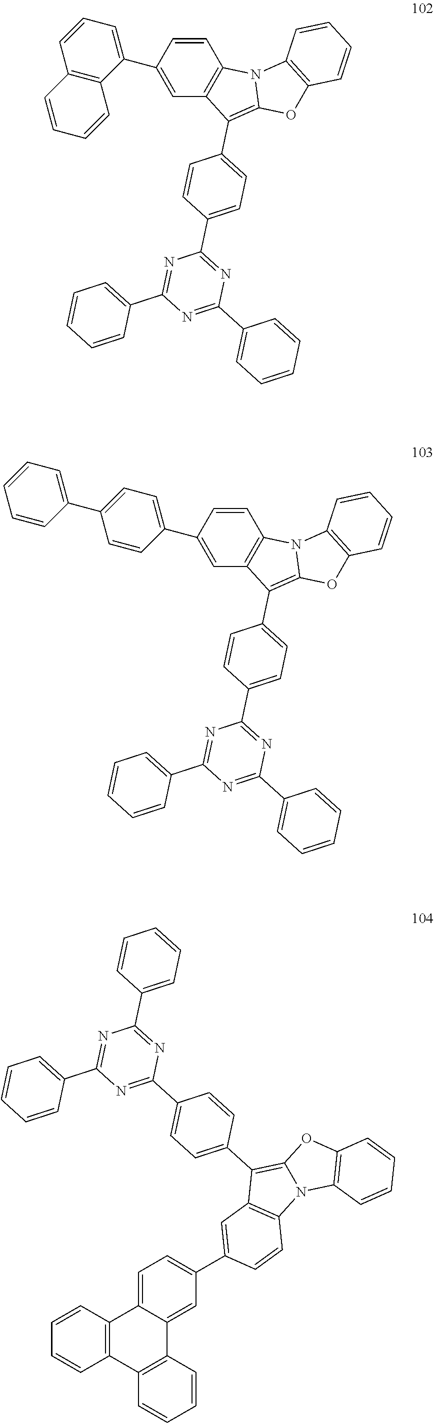 [SMILES: c1ccc(-c2nc(-c3ccccc3)nc(-c3ccc(-c4c5cc(-c6cccc7ccccc67)ccc5n5c4oc4ccccc45)cc3)n2)cc1 ; c1ccc(-c2ccc(-c3ccc4c(c3)c(-c3ccc(-c5nc(-c6ccccc6)nc(-c6ccccc6)n5)cc3)c3oc5ccccc5n34)cc2)cc1 ; c1ccc(-c2nc(-c3ccccc3)nc(-c3ccc(-c4c5cc(-c6ccc7c8ccccc8c8ccccc8c7c6)ccc5n5c4oc4ccccc45)cc3)n2)cc1]

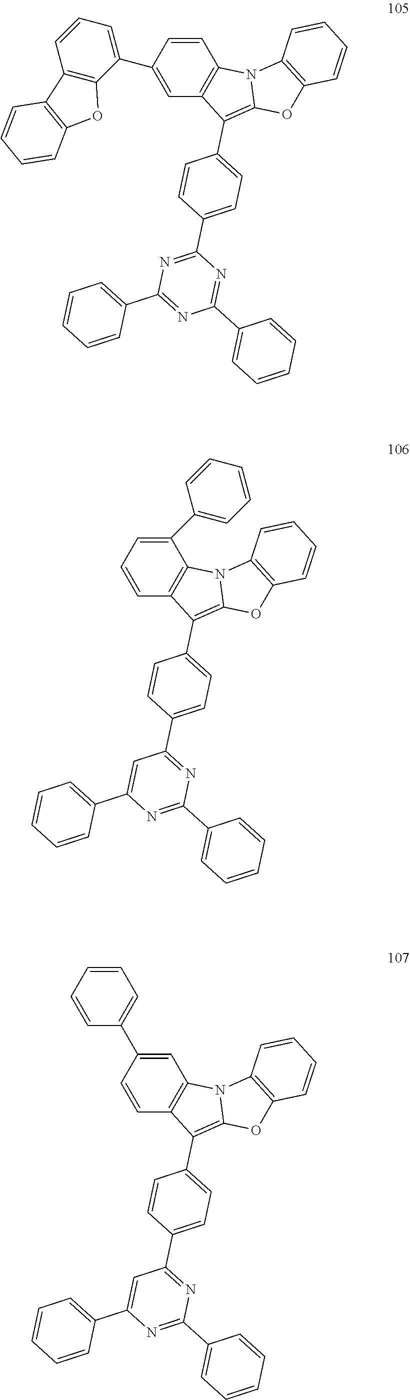 [SMILES: c1ccc(-c2nc(-c3ccccc3)nc(-c3ccc(-c4c5cc(-c6cccc7c6oc6ccccc67)ccc5n5c4oc4ccccc45)cc3)n2)cc1 ; c1ccc(-c2cc(-c3ccc(-c4c5cccc(-c6ccccc6)c5n5c4oc4ccccc45)cc3)nc(-c3ccccc3)n2)cc1 ; c1ccc(-c2ccc3c(-c4ccc(-c5cc(-c6ccccc6)nc(-c6ccccc6)n5)cc4)c4oc5ccccc5n4c3c2)cc1]

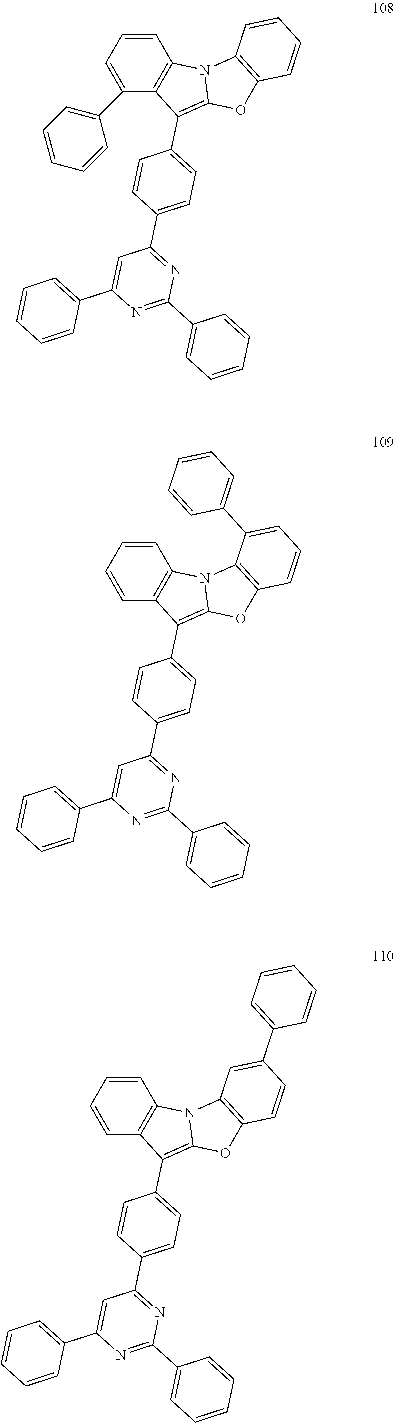 [SMILES: c1ccc(-c2cc(-c3ccc(-c4c5c(-c6ccccc6)cccc5n5c4oc4ccccc45)cc3)nc(-c3ccccc3)n2)cc1 ; c1ccc(-c2cc(-c3ccc(-c4c5ccccc5n5c4oc4cccc(-c6ccccc6)c45)cc3)nc(-c3ccccc3)n2)cc1 ; c1ccc(-c2ccc3oc4c(-c5ccc(-c6cc(-c7ccccc7)nc(-c7ccccc7)n6)cc5)c5ccccc5n4c3c2)cc1]

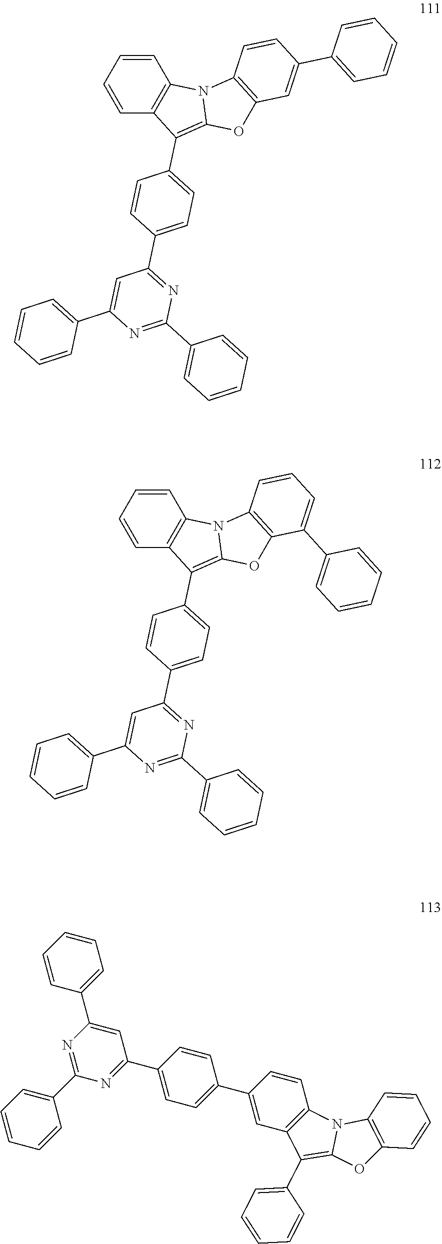 [SMILES: c1ccc(-c2ccc3c(c2)oc2c(-c4ccc(-c5cc(-c6ccccc6)nc(-c6ccccc6)n5)cc4)c4ccccc4n23)cc1 ; c1ccc(-c2cc(-c3ccc(-c4c5ccccc5n5c4oc4c(-c6ccccc6)cccc45)cc3)nc(-c3ccccc3)n2)cc1 ; c1ccc(-c2cc(-c3ccc(-c4ccc5c(c4)c(-c4ccccc4)c4oc6ccccc6n45)cc3)nc(-c3ccccc3)n2)cc1]

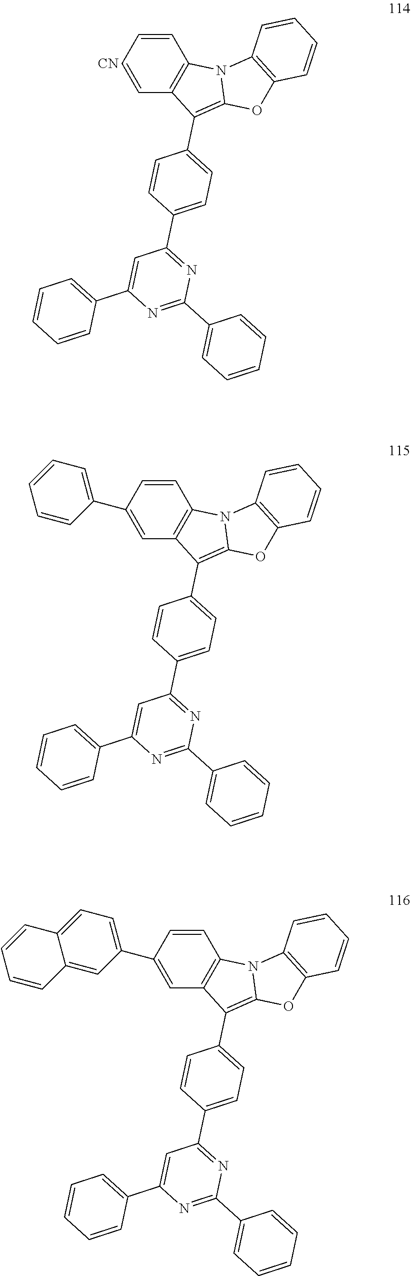

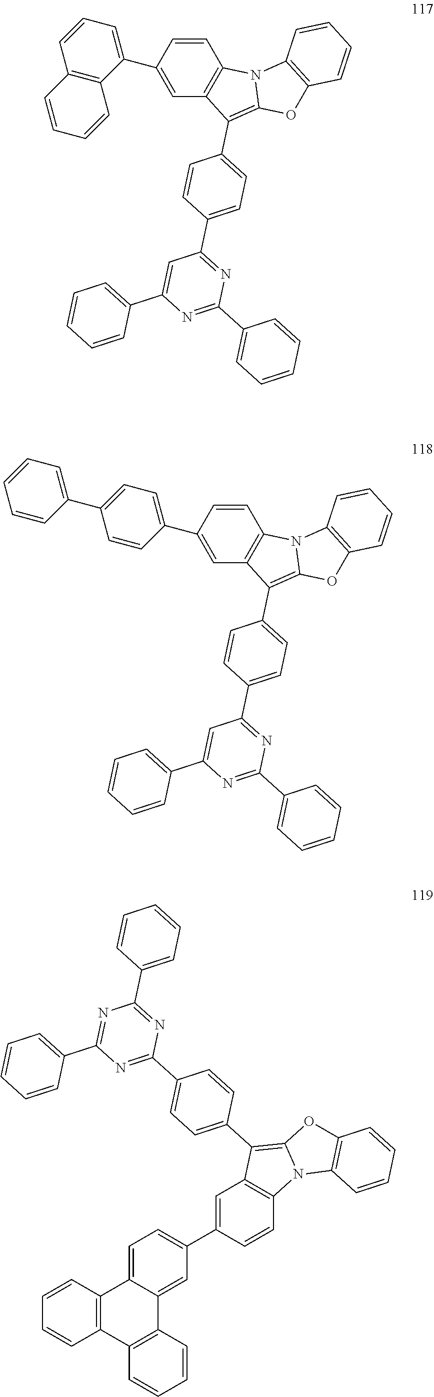 [SMILES: c1ccc(-c2cc(-c3ccc(-c4c5cc(-c6cccc7ccccc67)ccc5n5c4oc4ccccc45)cc3)nc(-c3ccccc3)n2)cc1 ; c1ccc(-c2ccc(-c3ccc4c(c3)c(-c3ccc(-c5cc(-c6ccccc6)nc(-c6ccccc6)n5)cc3)c3oc5ccccc5n34)cc2)cc1 ; c1ccc(-c2nc(-c3ccccc3)nc(-c3ccc(-c4c5cc(-c6ccc7c8ccccc8c8ccccc8c7c6)ccc5n5c4oc4ccccc45)cc3)n2)cc1]

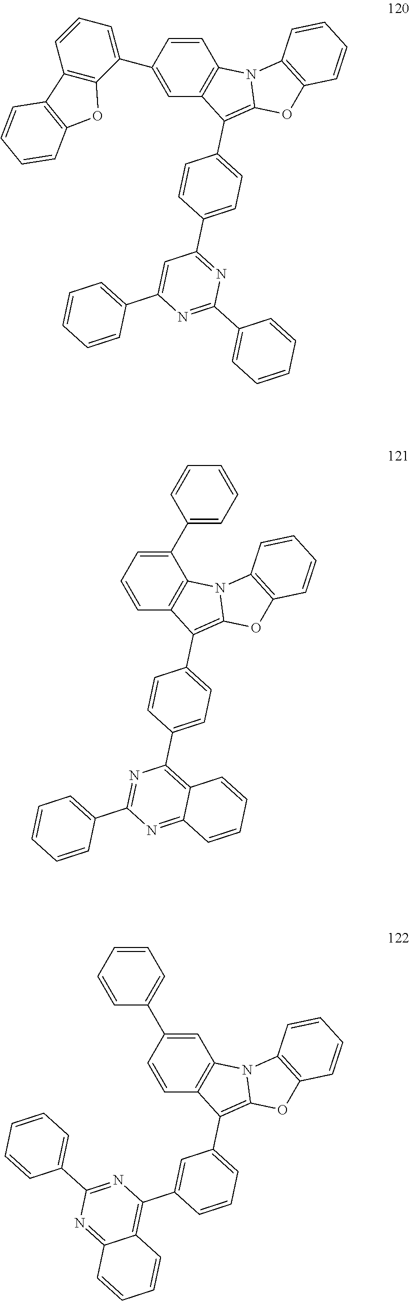 [SMILES: c1ccc(-c2cc(-c3ccc(-c4c5cc(-c6cccc7c6oc6ccccc67)ccc5n5c4oc4ccccc45)cc3)nc(-c3ccccc3)n2)cc1 ; c1ccc(-c2nc(-c3ccc(-c4c5cccc(-c6ccccc6)c5n5c4oc4ccccc45)cc3)c3ccccc3n2)cc1 ; c1ccc(-c2ccc3c(-c4cccc(-c5nc(-c6ccccc6)nc6ccccc56)c4)c4oc5ccccc5n4c3c2)cc1]

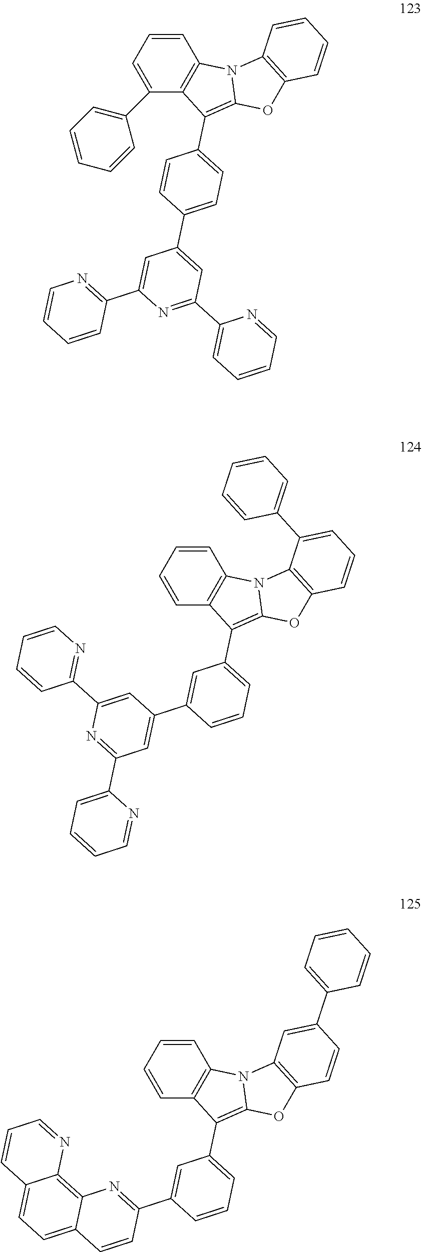 [SMILES: c1ccc(-c2cccc3c2c(-c2ccc(-c4cc(-c5ccccn5)nc(-c5ccccn5)c4)cc2)c2oc4ccccc4n23)cc1 ; c1ccc(-c2cccc3oc4c(-c5cccc(-c6cc(-c7ccccn7)nc(-c7ccccn7)c6)c5)c5ccccc5n4c23)cc1 ; c1ccc(-c2ccc3oc4c(-c5cccc(-c6ccc7ccc8cccnc8c7n6)c5)c5ccccc5n4c3c2)cc1]

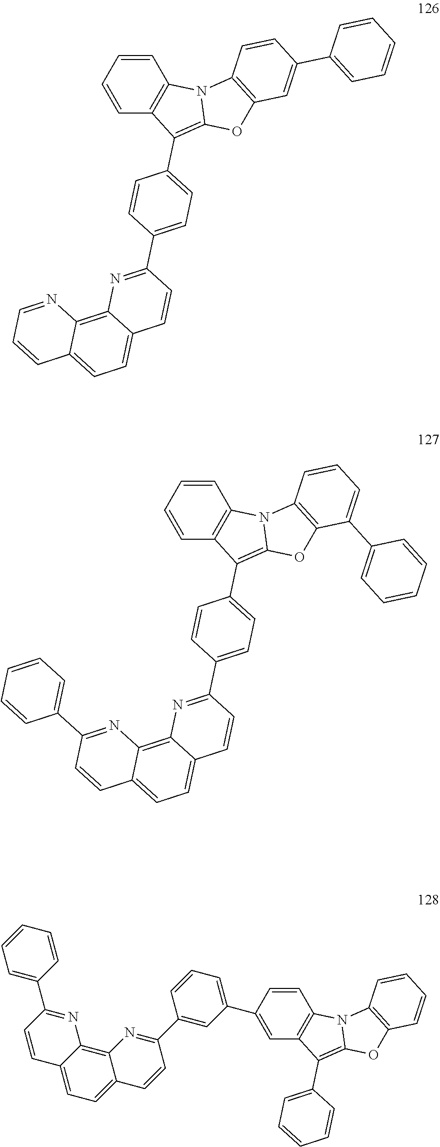 [SMILES: c1ccc(-c2ccc3c(c2)oc2c(-c4ccc(-c5ccc6ccc7cccnc7c6n5)cc4)c4ccccc4n23)cc1 ; c1ccc(-c2ccc3ccc4ccc(-c5ccc(-c6c7ccccc7n7c6oc6c(-c8ccccc8)cccc67)cc5)nc4c3n2)cc1 ; c1ccc(-c2ccc3ccc4ccc(-c5cccc(-c6ccc7c(c6)c(-c6ccccc6)c6oc8ccccc8n67)c5)nc4c3n2)cc1]

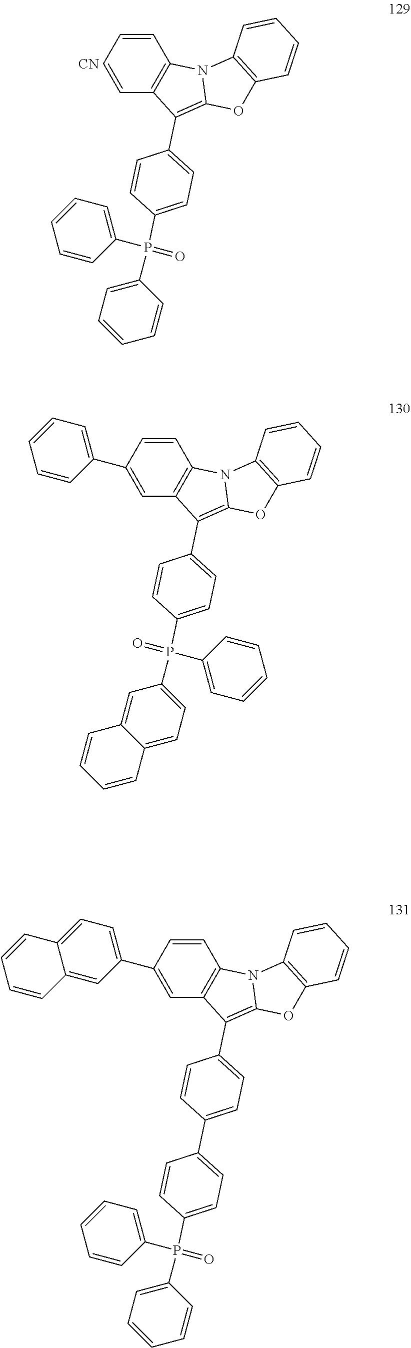

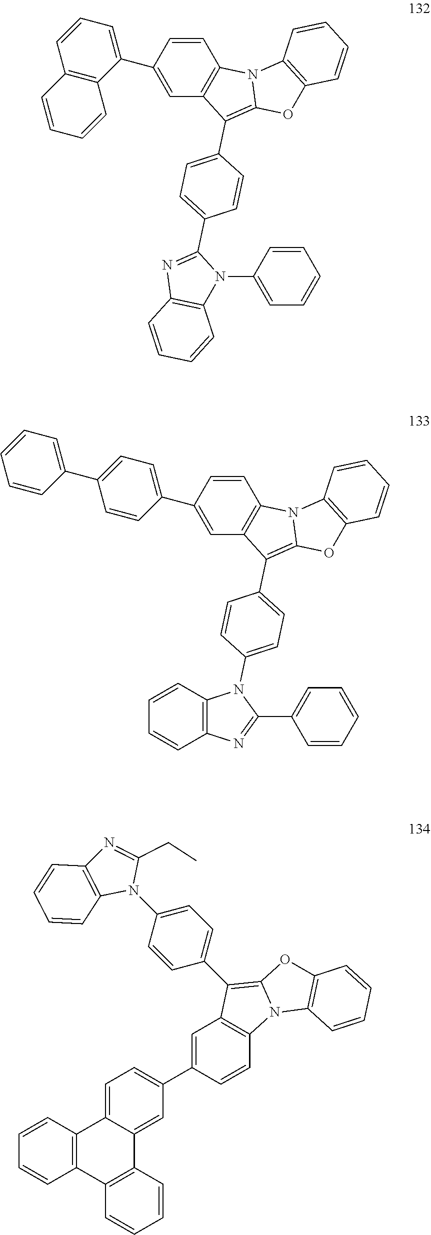 [SMILES: c1ccc(-n2c(-c3ccc(-c4c5cc(-c6cccc7ccccc67)ccc5n5c4oc4ccccc45)cc3)nc3ccccc32)cc1 ; c1ccc(-c2ccc(-c3ccc4c(c3)c(-c3ccc(-n5c(-c6ccccc6)nc6ccccc65)cc3)c3oc5ccccc5n34)cc2)cc1 ; CCc1nc2ccccc2n1-c1ccc(-c2c3cc(-c4ccc5c6ccccc6c6ccccc6c5c4)ccc3n3c2oc2ccccc23)cc1]

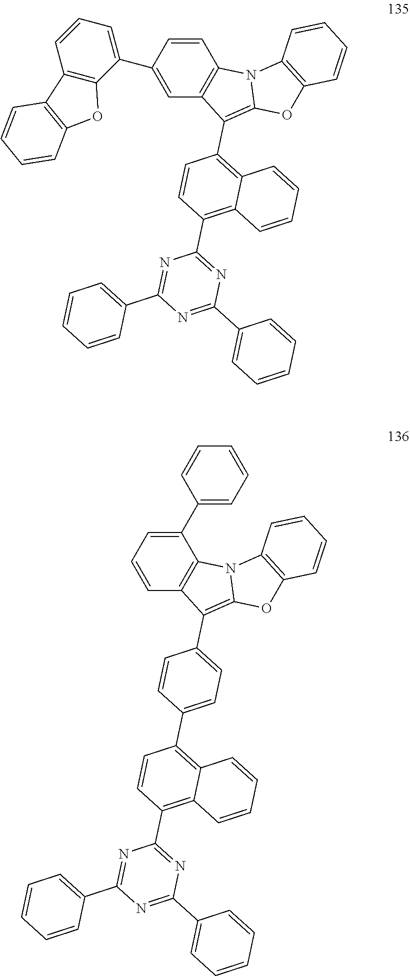 [SMILES: c1ccc(-c2nc(-c3ccccc3)nc(-c3ccc(-c4c5cc(-c6cccc7c6oc6ccccc67)ccc5n5c4oc4ccccc45)c4ccccc34)n2)cc1 ; c1ccc(-c2nc(-c3ccccc3)nc(-c3ccc(-c4ccc(-c5c6cccc(-c7ccccc7)c6n6c5oc5ccccc56)cc4)c4ccccc34)n2)cc1]

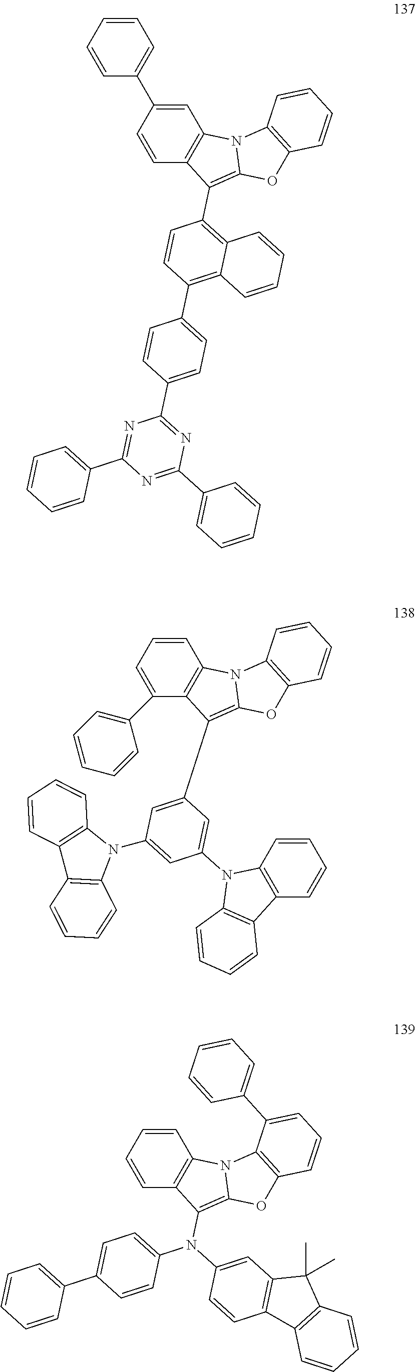 [SMILES: c1ccc(-c2ccc3c(-c4ccc(-c5ccc(-c6nc(-c7ccccc7)nc(-c7ccccc7)n6)cc5)c5ccccc45)c4oc5ccccc5n4c3c2)cc1 ; c1ccc(-c2cccc3c2c(-c2cc(-n4c5ccccc5c5ccccc54)cc(-n4c5ccccc5c5ccccc54)c2)c2oc4ccccc4n23)cc1 ; CC1(C)c2ccccc2-c2ccc(N(c3ccc(-c4ccccc4)cc3)c3c4ccccc4n4c3oc3cccc(-c5ccccc5)c34)cc21]

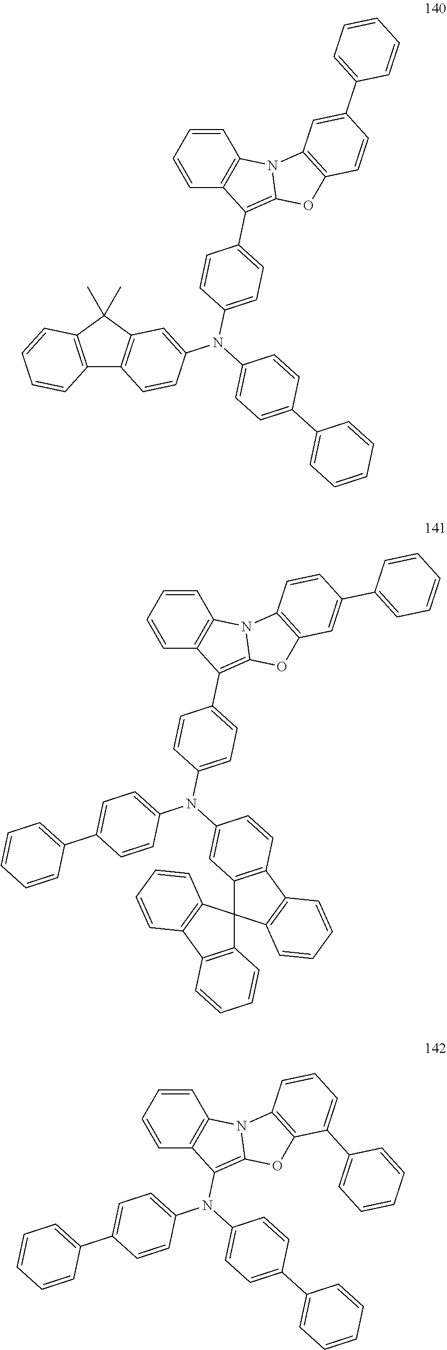 [SMILES: CC1(C)c2ccccc2-c2ccc(N(c3ccc(-c4ccccc4)cc3)c3ccc(-c4c5ccccc5n5c4oc4ccc(-c6ccccc6)cc45)cc3)cc21 ; c1ccc(-c2ccc(N(c3ccc(-c4c5ccccc5n5c4oc4cc(-c6ccccc6)ccc45)cc3)c3ccc4c(c3)C3(c5ccccc5-c5ccccc53)c3ccccc3-4)cc2)cc1 ; c1ccc(-c2ccc(N(c3ccc(-c4ccccc4)cc3)c3c4ccccc4n4c3oc3c(-c5ccccc5)cccc34)cc2)cc1]

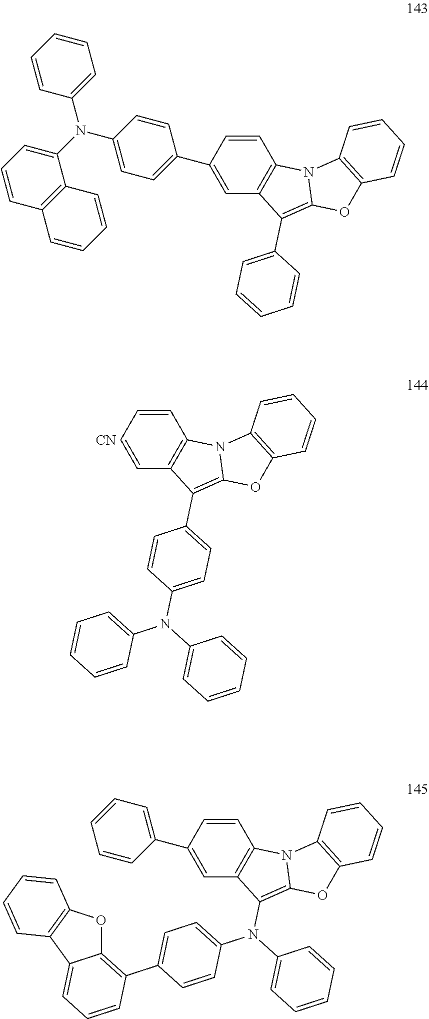

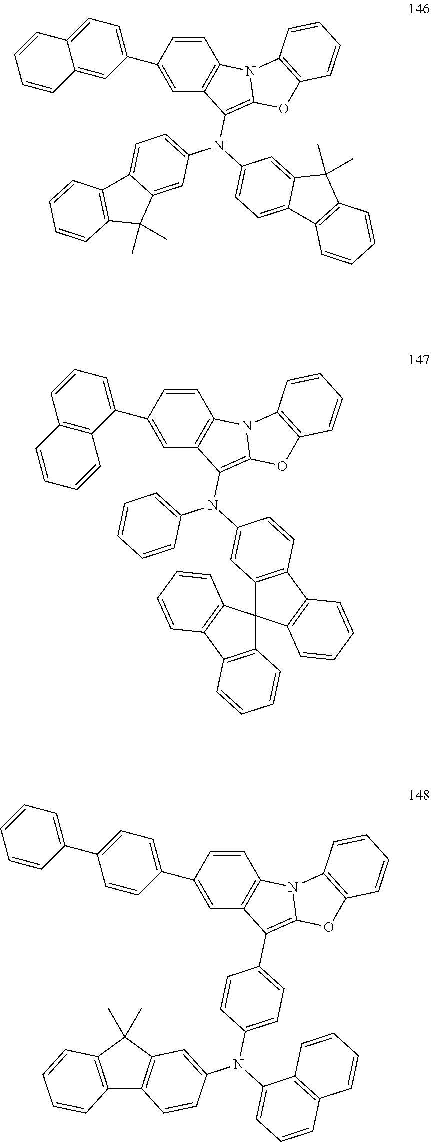 [SMILES: CC1(C)c2ccccc2-c2ccc(N(c3ccc4c(c3)C(C)(C)c3ccccc3-4)c3c4cc(-c5ccc6ccccc6c5)ccc4n4c3oc3ccccc34)cc21 ; c1ccc(N(c2ccc3c(c2)C2(c4ccccc4-c4ccccc42)c2ccccc2-3)c2c3cc(-c4cccc5ccccc45)ccc3n3c2oc2ccccc23)cc1 ; CC1(C)c2ccccc2-c2ccc(N(c3ccc(-c4c5cc(-c6ccc(-c7ccccc7)cc6)ccc5n5c4oc4ccccc45)cc3)c3cccc4ccccc34)cc21]

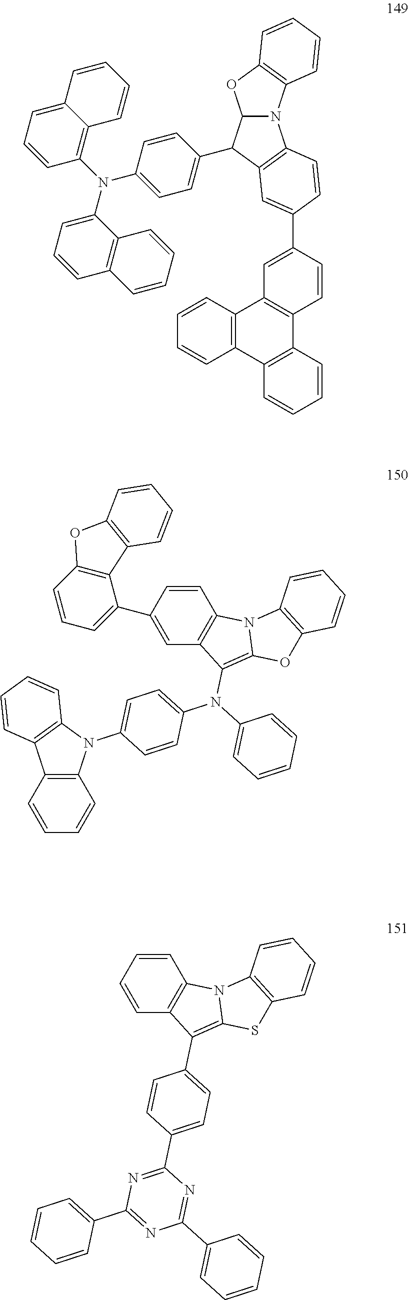 [SMILES: c1ccc2c(c1)OC1C(c3ccc(N(c4cccc5ccccc45)c4cccc5ccccc45)cc3)c3cc(-c4ccc5c6ccccc6c6ccccc6c5c4)ccc3N21 ; c1ccc(N(c2ccc(-n3c4ccccc4c4ccccc43)cc2)c2c3cc(-c4cccc5oc6ccccc6c45)ccc3n3c2oc2ccccc23)cc1 ; c1ccc(-c2nc(-c3ccccc3)nc(-c3ccc(-c4c5ccccc5n5c4sc4ccccc45)cc3)n2)cc1]

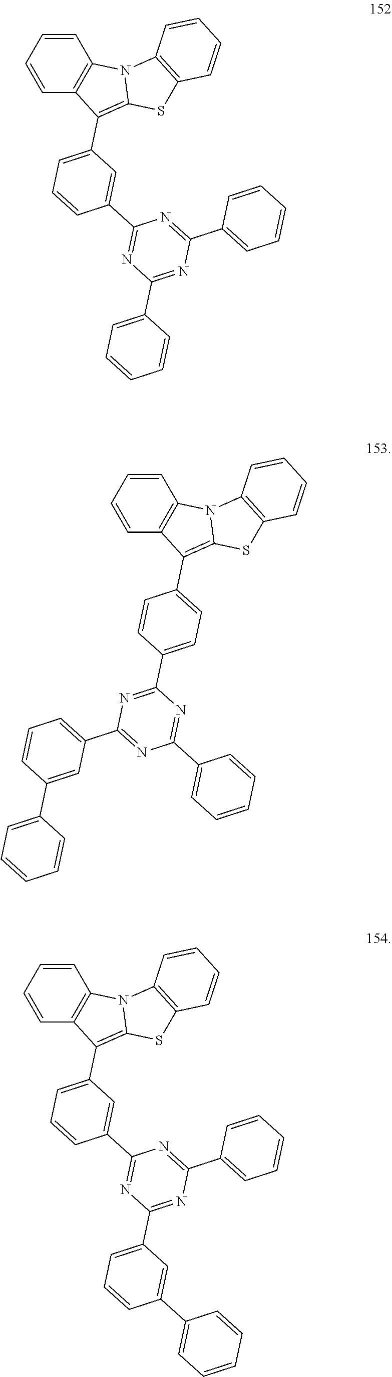 [SMILES: c1ccc(-c2nc(-c3ccccc3)nc(-c3cccc(-c4c5ccccc5n5c4sc4ccccc45)c3)n2)cc1 ; c1ccc(-c2cccc(-c3nc(-c4ccccc4)nc(-c4ccc(-c5c6ccccc6n6c5sc5ccccc56)cc4)n3)c2)cc1 ; c1ccc(-c2cccc(-c3nc(-c4ccccc4)nc(-c4cccc(-c5c6ccccc6n6c5sc5ccccc56)c4)n3)c2)cc1]

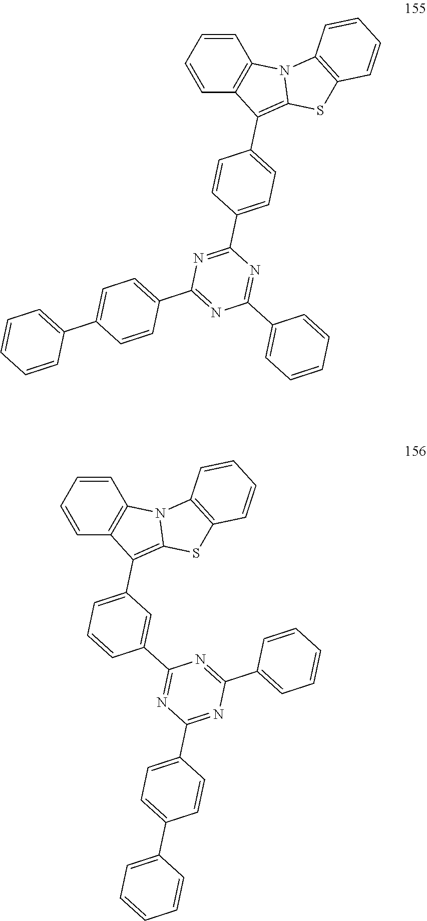 [SMILES: c1ccc(-c2ccc(-c3nc(-c4ccccc4)nc(-c4ccc(-c5c6ccccc6n6c5sc5ccccc56)cc4)n3)cc2)cc1 ; c1ccc(-c2ccc(-c3nc(-c4ccccc4)nc(-c4cccc(-c5c6ccccc6n6c5sc5ccccc56)c4)n3)cc2)cc1]

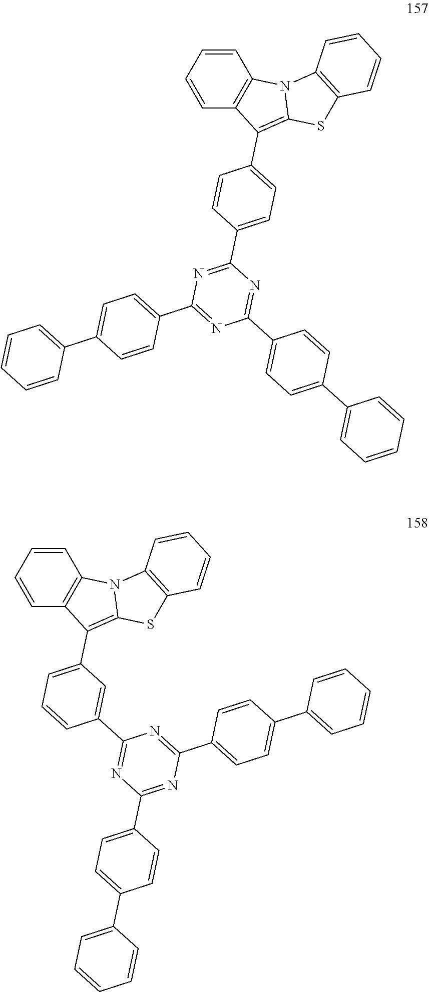 [SMILES: c1ccc(-c2ccc(-c3nc(-c4ccc(-c5ccccc5)cc4)nc(-c4ccc(-c5c6ccccc6n6c5sc5ccccc56)cc4)n3)cc2)cc1 ; c1ccc(-c2ccc(-c3nc(-c4ccc(-c5ccccc5)cc4)nc(-c4cccc(-c5c6ccccc6n6c5sc5ccccc56)c4)n3)cc2)cc1]

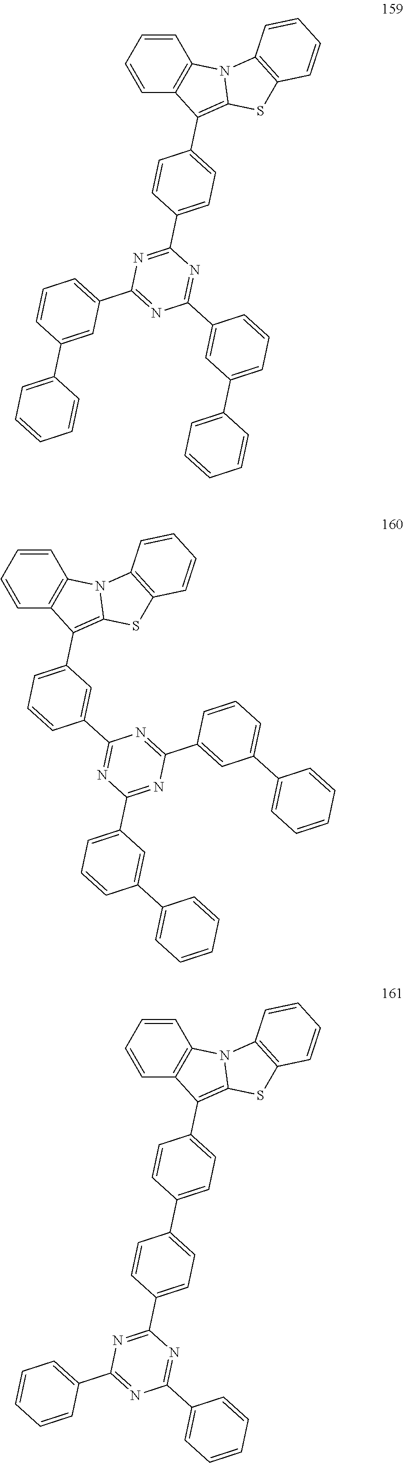 [SMILES: c1ccc(-c2cccc(-c3nc(-c4ccc(-c5c6ccccc6n6c5sc5ccccc56)cc4)nc(-c4cccc(-c5ccccc5)c4)n3)c2)cc1 ; c1ccc(-c2cccc(-c3nc(-c4cccc(-c5ccccc5)c4)nc(-c4cccc(-c5c6ccccc6n6c5sc5ccccc56)c4)n3)c2)cc1 ; c1ccc(-c2nc(-c3ccccc3)nc(-c3ccc(-c4ccc(-c5c6ccccc6n6c5sc5ccccc56)cc4)cc3)n2)cc1]

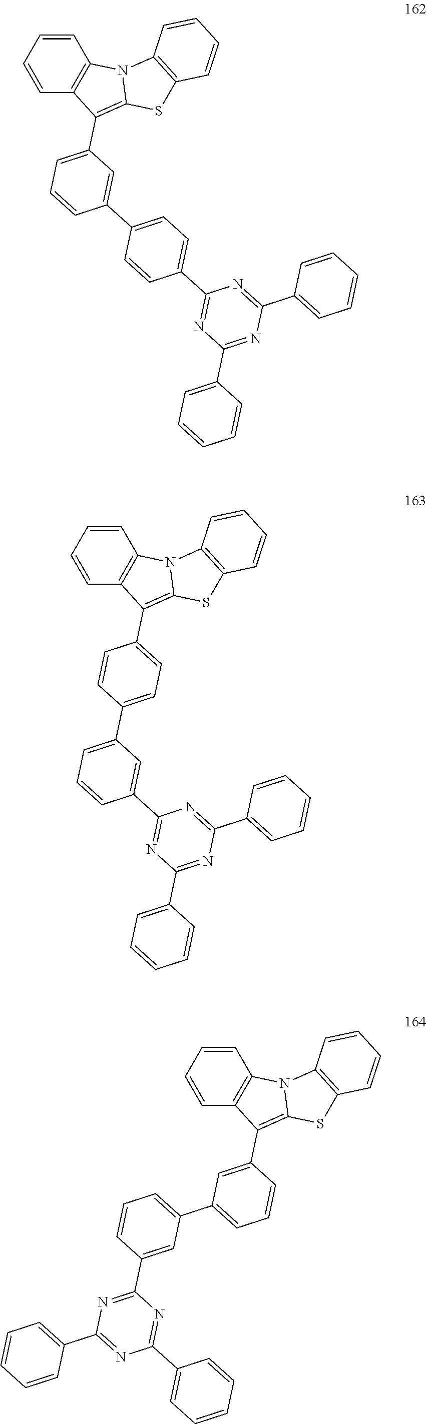 [SMILES: c1ccc(-c2nc(-c3ccccc3)nc(-c3ccc(-c4cccc(-c5c6ccccc6n6c5sc5ccccc56)c4)cc3)n2)cc1 ; c1ccc(-c2nc(-c3ccccc3)nc(-c3cccc(-c4ccc(-c5c6ccccc6n6c5sc5ccccc56)cc4)c3)n2)cc1 ; c1ccc(-c2nc(-c3ccccc3)nc(-c3cccc(-c4cccc(-c5c6ccccc6n6c5sc5ccccc56)c4)c3)n2)cc1]

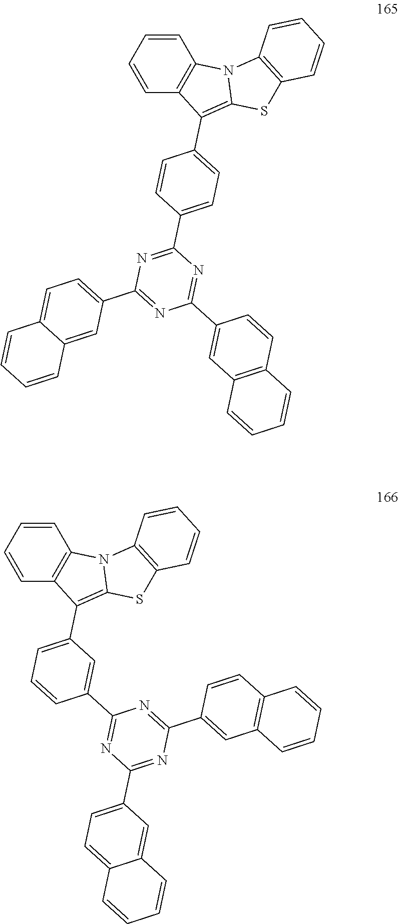 [SMILES: c1ccc2cc(-c3nc(-c4ccc(-c5c6ccccc6n6c5sc5ccccc56)cc4)nc(-c4ccc5ccccc5c4)n3)ccc2c1 ; c1cc(-c2nc(-c3ccc4ccccc4c3)nc(-c3ccc4ccccc4c3)n2)cc(-c2c3ccccc3n3c2sc2ccccc23)c1]

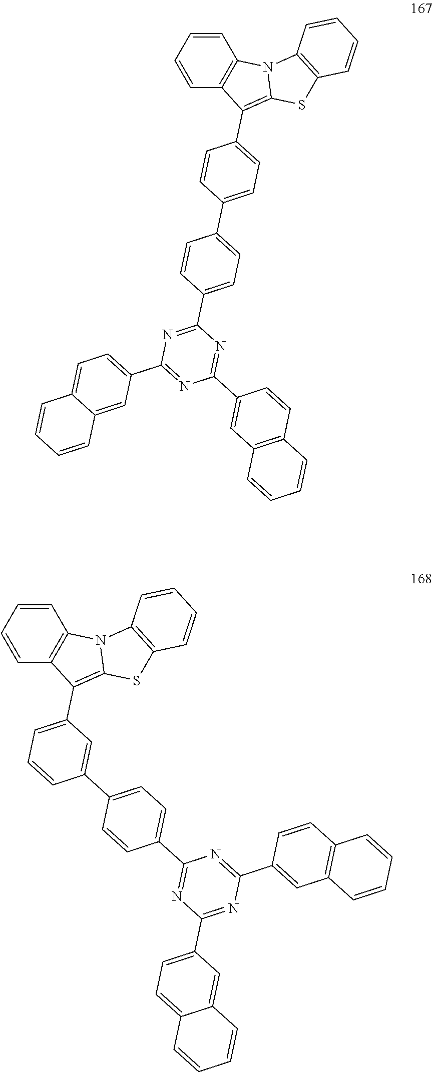 [SMILES: c1ccc2cc(-c3nc(-c4ccc(-c5ccc(-c6c7ccccc7n7c6sc6ccccc67)cc5)cc4)nc(-c4ccc5ccccc5c4)n3)ccc2c1 ; c1cc(-c2ccc(-c3nc(-c4ccc5ccccc5c4)nc(-c4ccc5ccccc5c4)n3)cc2)cc(-c2c3ccccc3n3c2sc2ccccc23)c1]

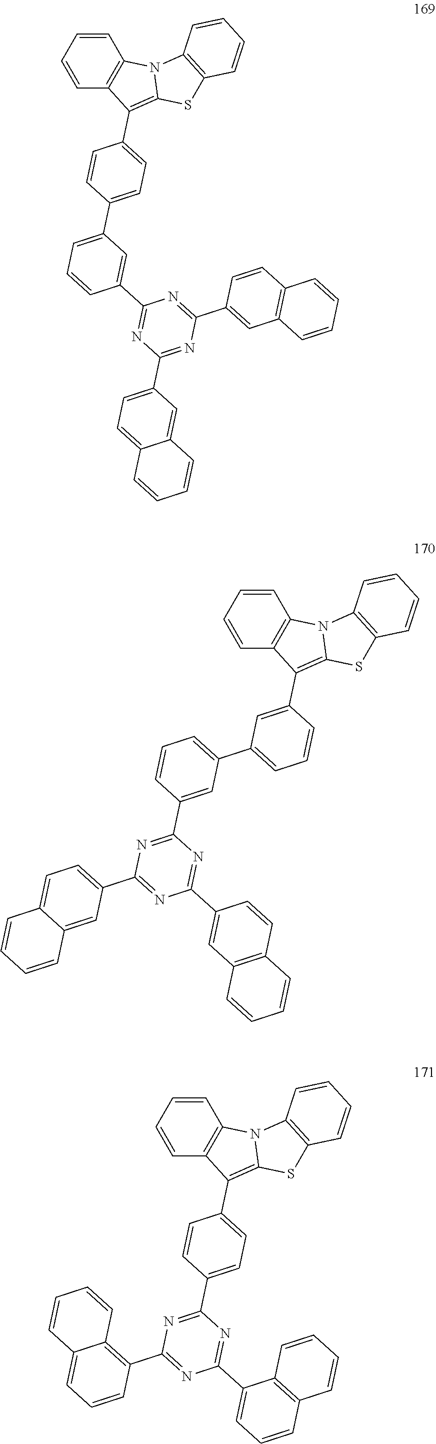 [SMILES: c1cc(-c2ccc(-c3c4ccccc4n4c3sc3ccccc34)cc2)cc(-c2nc(-c3ccc4ccccc4c3)nc(-c3ccc4ccccc4c3)n2)c1 ; c1cc(-c2cccc(-c3c4ccccc4n4c3sc3ccccc34)c2)cc(-c2nc(-c3ccc4ccccc4c3)nc(-c3ccc4ccccc4c3)n2)c1 ; c1ccc2c(-c3nc(-c4ccc(-c5c6ccccc6n6c5sc5ccccc56)cc4)nc(-c4cccc5ccccc45)n3)cccc2c1]

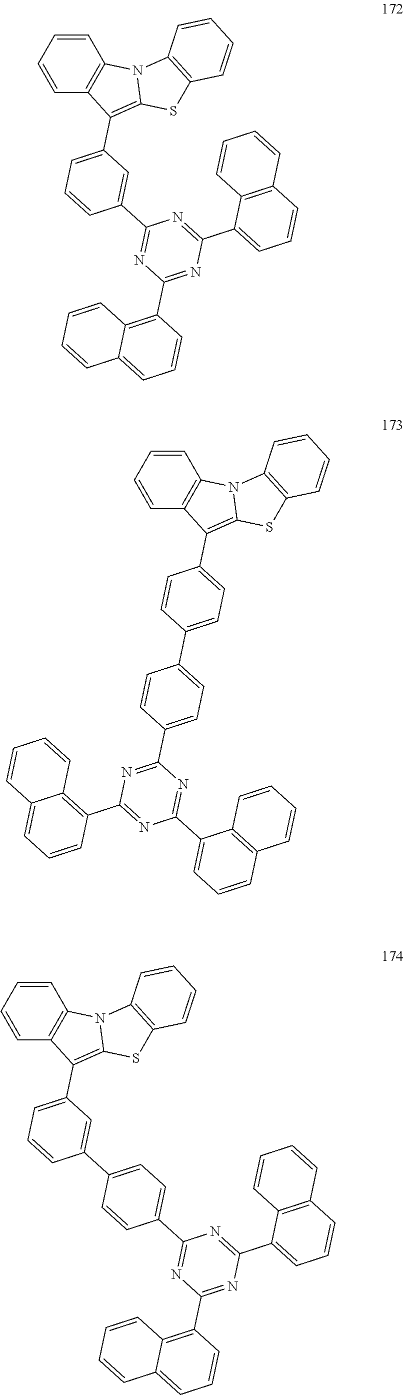 [SMILES: c1cc(-c2nc(-c3cccc4ccccc34)nc(-c3cccc4ccccc34)n2)cc(-c2c3ccccc3n3c2sc2ccccc23)c1 ; c1ccc2c(-c3nc(-c4ccc(-c5ccc(-c6c7ccccc7n7c6sc6ccccc67)cc5)cc4)nc(-c4cccc5ccccc45)n3)cccc2c1 ; c1cc(-c2ccc(-c3nc(-c4cccc5ccccc45)nc(-c4cccc5ccccc45)n3)cc2)cc(-c2c3ccccc3n3c2sc2ccccc23)c1]

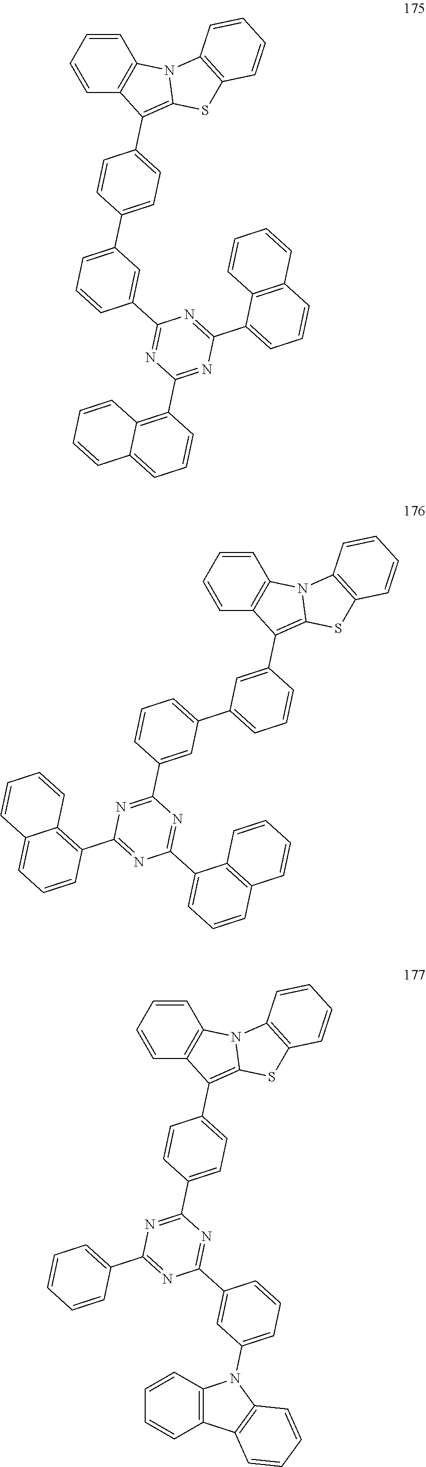 [SMILES: c1cc(-c2ccc(-c3c4ccccc4n4c3sc3ccccc34)cc2)cc(-c2nc(-c3cccc4ccccc34)nc(-c3cccc4ccccc34)n2)c1 ; c1cc(-c2cccc(-c3c4ccccc4n4c3sc3ccccc34)c2)cc(-c2nc(-c3cccc4ccccc34)nc(-c3cccc4ccccc34)n2)c1 ; c1ccc(-c2nc(-c3ccc(-c4c5ccccc5n5c4sc4ccccc45)cc3)nc(-c3cccc(-n4c5ccccc5c5ccccc54)c3)n2)cc1]

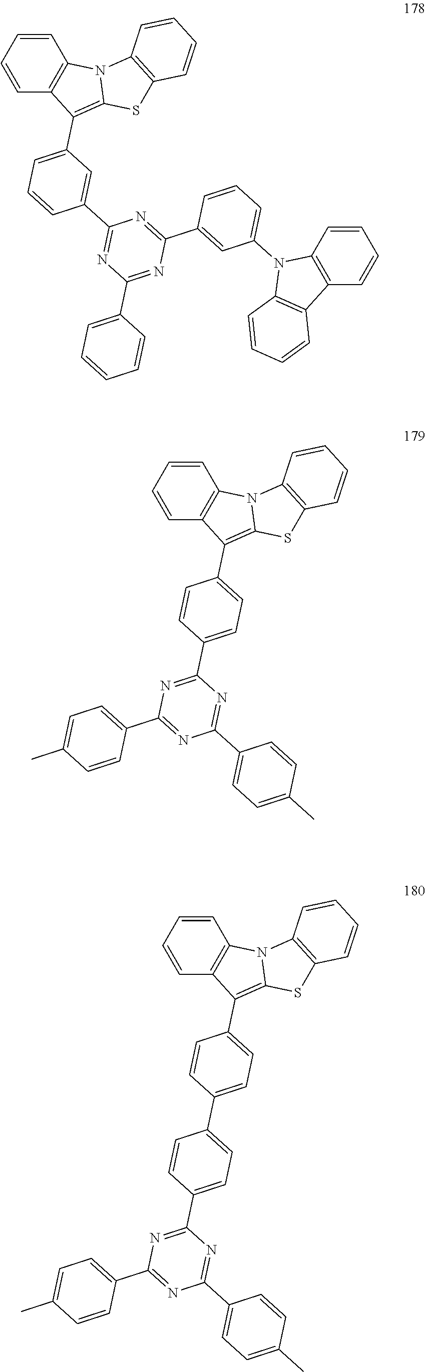 [SMILES: c1ccc(-c2nc(-c3cccc(-c4c5ccccc5n5c4sc4ccccc45)c3)nc(-c3cccc(-n4c5ccccc5c5ccccc54)c3)n2)cc1 ; Cc1ccc(-c2nc(-c3ccc(C)cc3)nc(-c3ccc(-c4c5ccccc5n5c4sc4ccccc45)cc3)n2)cc1 ; Cc1ccc(-c2nc(-c3ccc(C)cc3)nc(-c3ccc(-c4ccc(-c5c6ccccc6n6c5sc5ccccc56)cc4)cc3)n2)cc1]

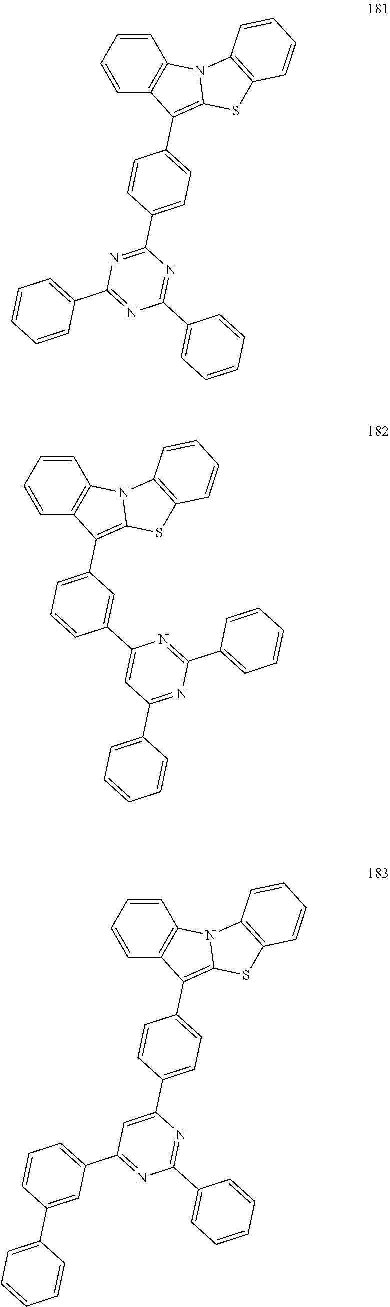 [SMILES: c1ccc(-c2nc(-c3ccccc3)nc(-c3ccc(-c4c5ccccc5n5c4sc4ccccc45)cc3)n2)cc1 ; c1ccc(-c2cc(-c3cccc(-c4c5ccccc5n5c4sc4ccccc45)c3)nc(-c3ccccc3)n2)cc1 ; c1ccc(-c2cccc(-c3cc(-c4ccc(-c5c6ccccc6n6c5sc5ccccc56)cc4)nc(-c4ccccc4)n3)c2)cc1]

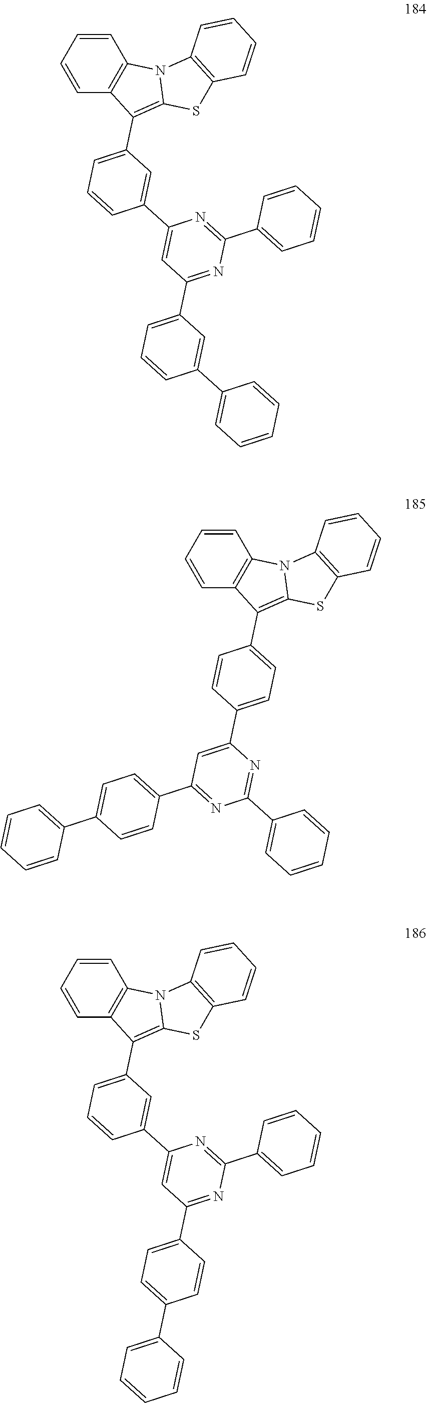 [SMILES: c1ccc(-c2cccc(-c3cc(-c4cccc(-c5c6ccccc6n6c5sc5ccccc56)c4)nc(-c4ccccc4)n3)c2)cc1 ; c1ccc(-c2ccc(-c3cc(-c4ccc(-c5c6ccccc6n6c5sc5ccccc56)cc4)nc(-c4ccccc4)n3)cc2)cc1 ; c1ccc(-c2ccc(-c3cc(-c4cccc(-c5c6ccccc6n6c5sc5ccccc56)c4)nc(-c4ccccc4)n3)cc2)cc1]

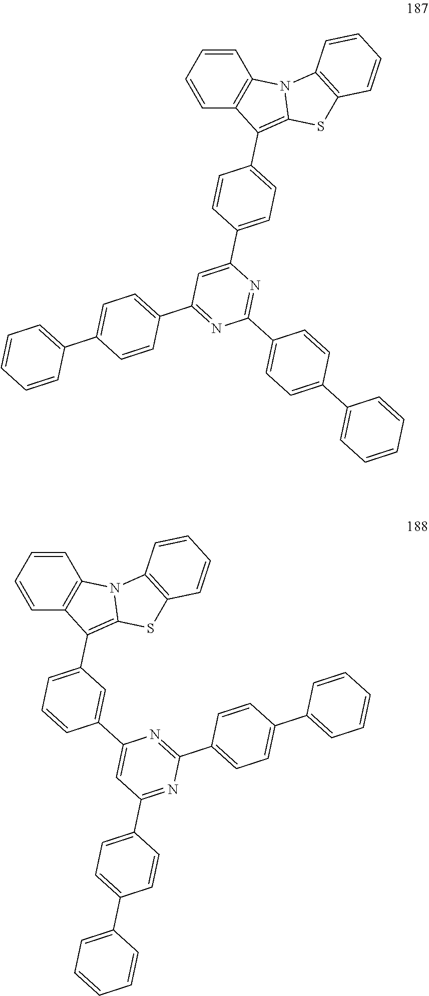 [SMILES: c1ccc(-c2ccc(-c3cc(-c4ccc(-c5c6ccccc6n6c5sc5ccccc56)cc4)nc(-c4ccc(-c5ccccc5)cc4)n3)cc2)cc1 ; c1ccc(-c2ccc(-c3cc(-c4cccc(-c5c6ccccc6n6c5sc5ccccc56)c4)nc(-c4ccc(-c5ccccc5)cc4)n3)cc2)cc1]

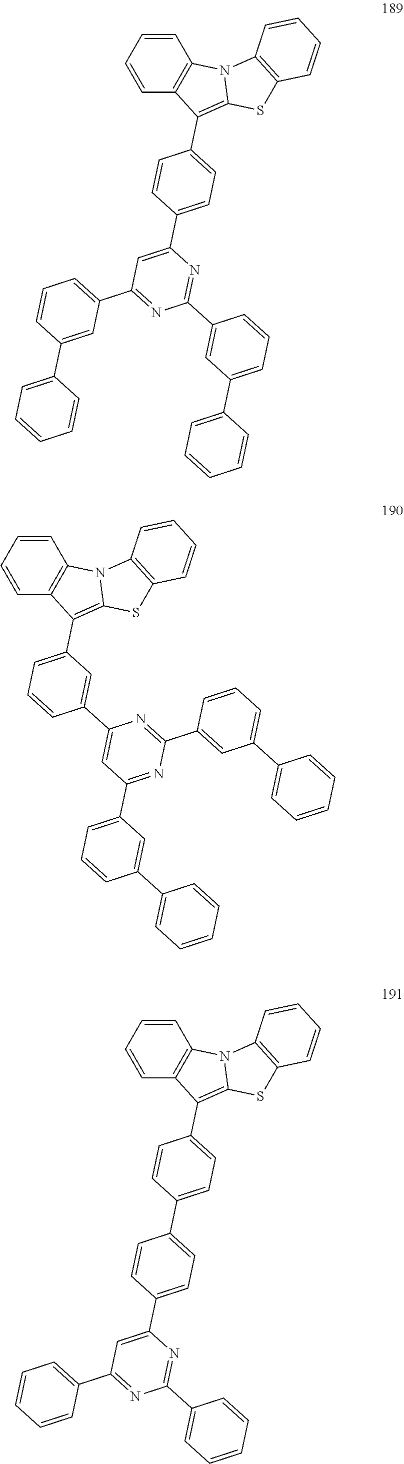 [SMILES: c1ccc(-c2cccc(-c3cc(-c4ccc(-c5c6ccccc6n6c5sc5ccccc56)cc4)nc(-c4cccc(-c5ccccc5)c4)n3)c2)cc1 ; c1ccc(-c2cccc(-c3cc(-c4cccc(-c5c6ccccc6n6c5sc5ccccc56)c4)nc(-c4cccc(-c5ccccc5)c4)n3)c2)cc1 ; c1ccc(-c2cc(-c3ccc(-c4ccc(-c5c6ccccc6n6c5sc5ccccc56)cc4)cc3)nc(-c3ccccc3)n2)cc1]

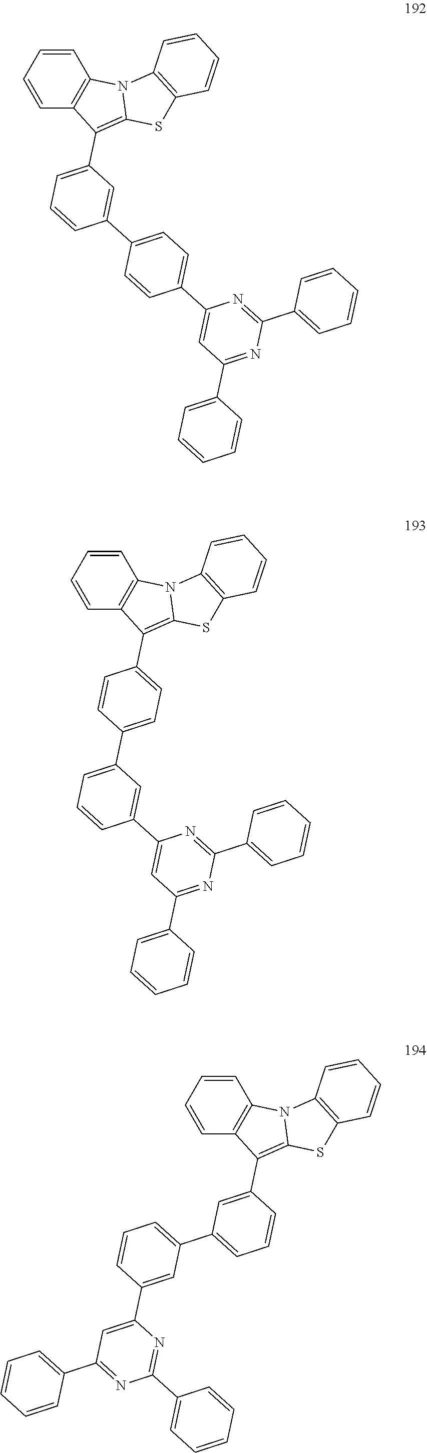 [SMILES: c1ccc(-c2cc(-c3ccc(-c4cccc(-c5c6ccccc6n6c5sc5ccccc56)c4)cc3)nc(-c3ccccc3)n2)cc1 ; c1ccc(-c2cc(-c3cccc(-c4ccc(-c5c6ccccc6n6c5sc5ccccc56)cc4)c3)nc(-c3ccccc3)n2)cc1 ; c1ccc(-c2cc(-c3cccc(-c4cccc(-c5c6ccccc6n6c5sc5ccccc56)c4)c3)nc(-c3ccccc3)n2)cc1]

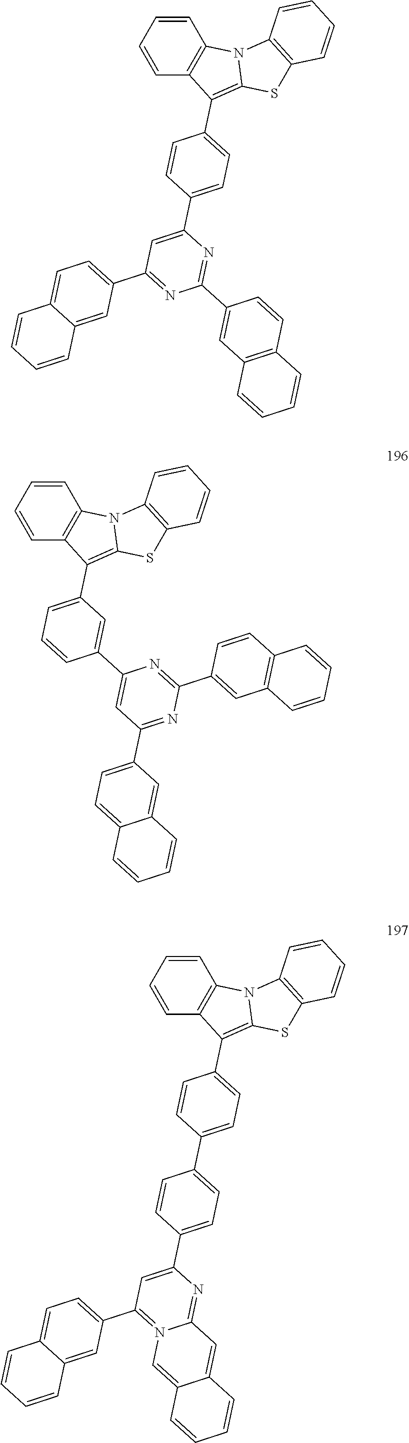

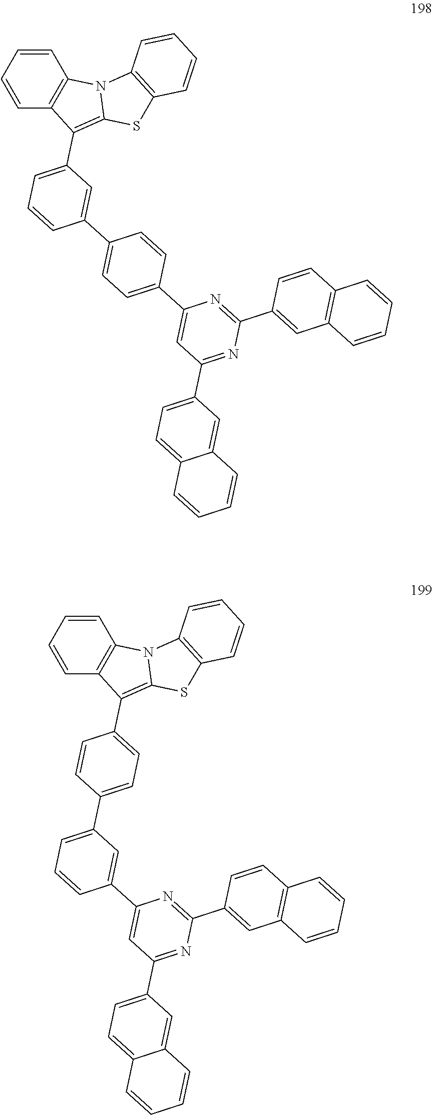 [SMILES: c1cc(-c2ccc(-c3cc(-c4ccc5ccccc5c4)nc(-c4ccc5ccccc5c4)n3)cc2)cc(-c2c3ccccc3n3c2sc2ccccc23)c1 ; c1cc(-c2ccc(-c3c4ccccc4n4c3sc3ccccc34)cc2)cc(-c2cc(-c3ccc4ccccc4c3)nc(-c3ccc4ccccc4c3)n2)c1]

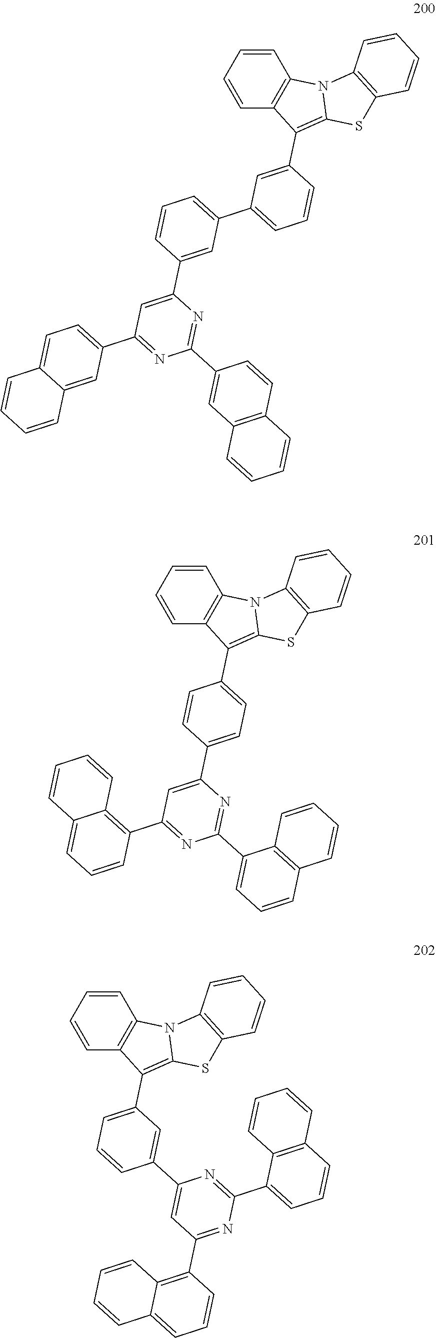 [SMILES: c1cc(-c2cccc(-c3c4ccccc4n4c3sc3ccccc34)c2)cc(-c2cc(-c3ccc4ccccc4c3)nc(-c3ccc4ccccc4c3)n2)c1 ; c1ccc2c(-c3cc(-c4ccc(-c5c6ccccc6n6c5sc5ccccc56)cc4)nc(-c4cccc5ccccc45)n3)cccc2c1 ; c1cc(-c2cc(-c3cccc4ccccc34)nc(-c3cccc4ccccc34)n2)cc(-c2c3ccccc3n3c2sc2ccccc23)c1]

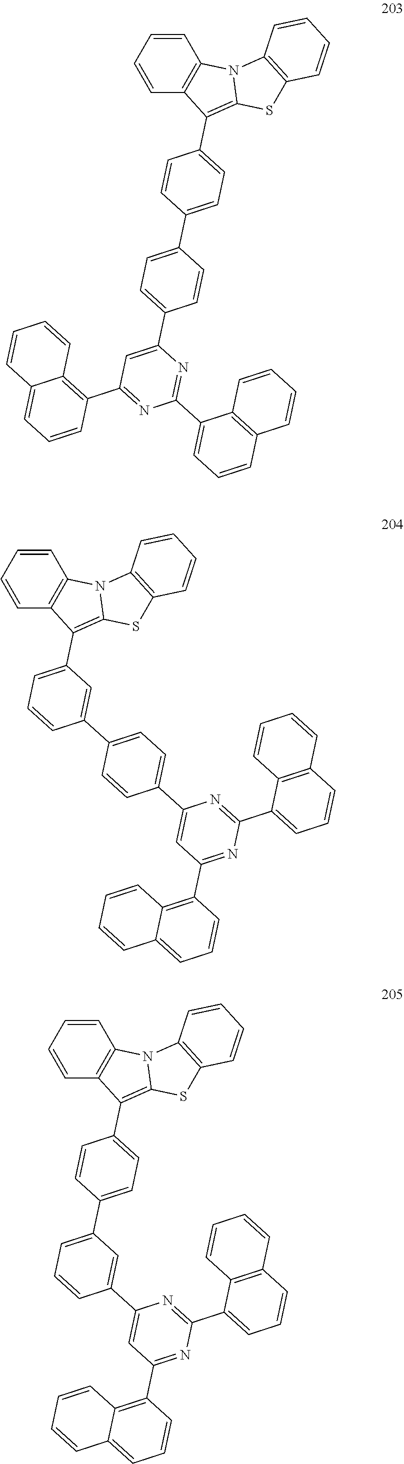 [SMILES: c1ccc2c(-c3cc(-c4ccc(-c5ccc(-c6c7ccccc7n7c6sc6ccccc67)cc5)cc4)nc(-c4cccc5ccccc45)n3)cccc2c1 ; c1cc(-c2ccc(-c3cc(-c4cccc5ccccc45)nc(-c4cccc5ccccc45)n3)cc2)cc(-c2c3ccccc3n3c2sc2ccccc23)c1 ; c1cc(-c2ccc(-c3c4ccccc4n4c3sc3ccccc34)cc2)cc(-c2cc(-c3cccc4ccccc34)nc(-c3cccc4ccccc34)n2)c1]

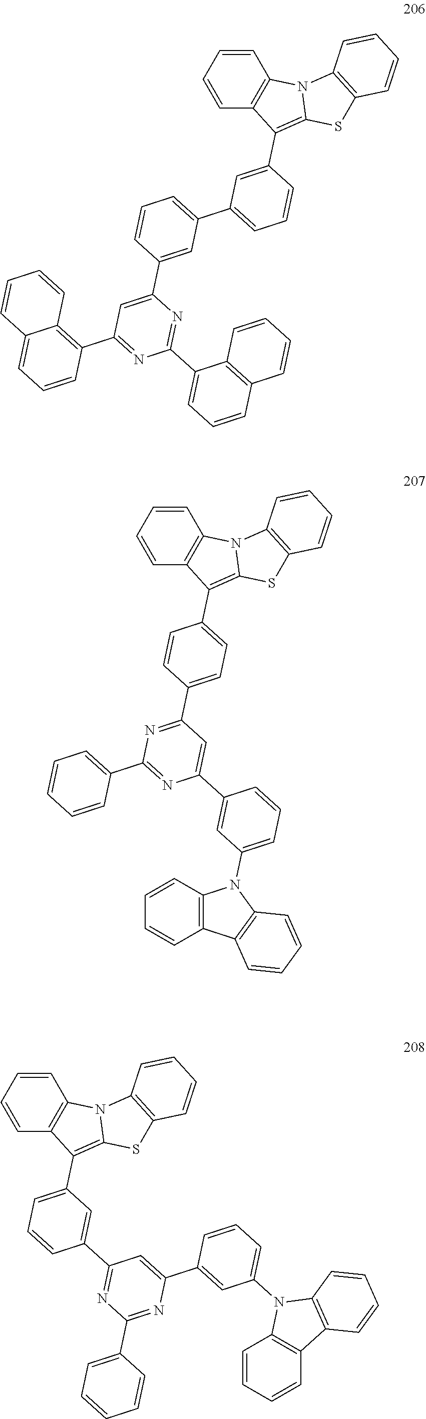 [SMILES: c1cc(-c2cccc(-c3c4ccccc4n4c3sc3ccccc34)c2)cc(-c2cc(-c3cccc4ccccc34)nc(-c3cccc4ccccc34)n2)c1 ; c1ccc(-c2nc(-c3ccc(-c4c5ccccc5n5c4sc4ccccc45)cc3)cc(-c3cccc(-n4c5ccccc5c5ccccc54)c3)n2)cc1 ; c1ccc(-c2nc(-c3cccc(-c4c5ccccc5n5c4sc4ccccc45)c3)cc(-c3cccc(-n4c5ccccc5c5ccccc54)c3)n2)cc1]

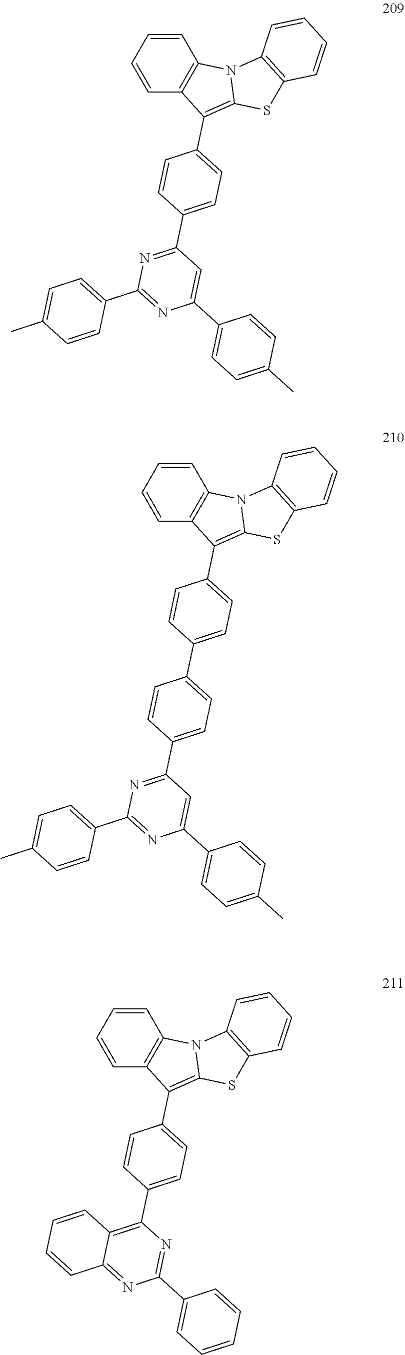 [SMILES: Cc1ccc(-c2cc(-c3ccc(-c4c5ccccc5n5c4sc4ccccc45)cc3)nc(-c3ccc(C)cc3)n2)cc1 ; Cc1ccc(-c2cc(-c3ccc(-c4ccc(-c5c6ccccc6n6c5sc5ccccc56)cc4)cc3)nc(-c3ccc(C)cc3)n2)cc1 ; c1ccc(-c2nc(-c3ccc(-c4c5ccccc5n5c4sc4ccccc45)cc3)c3ccccc3n2)cc1]

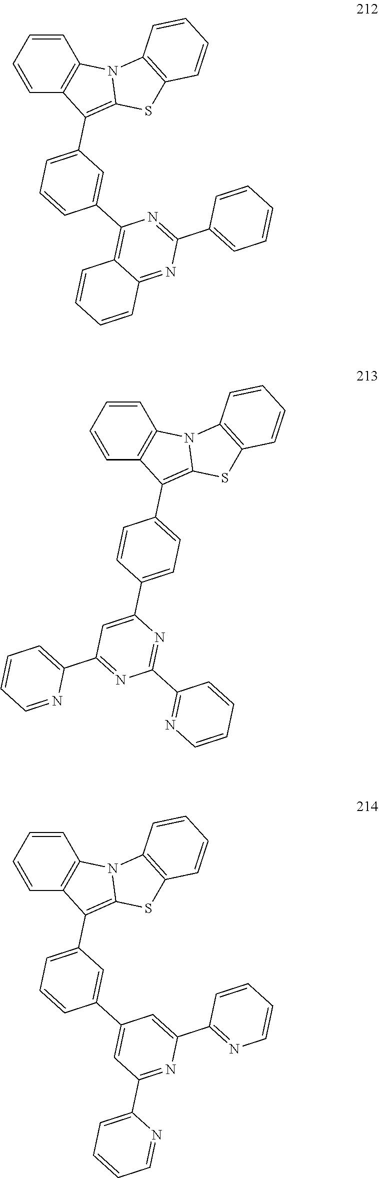 [SMILES: c1ccc(-c2nc(-c3cccc(-c4c5ccccc5n5c4sc4ccccc45)c3)c3ccccc3n2)cc1 ; c1ccc(-c2cc(-c3ccc(-c4c5ccccc5n5c4sc4ccccc45)cc3)nc(-c3ccccn3)n2)nc1 ; c1ccc(-c2cc(-c3cccc(-c4c5ccccc5n5c4sc4ccccc45)c3)cc(-c3ccccn3)n2)nc1]

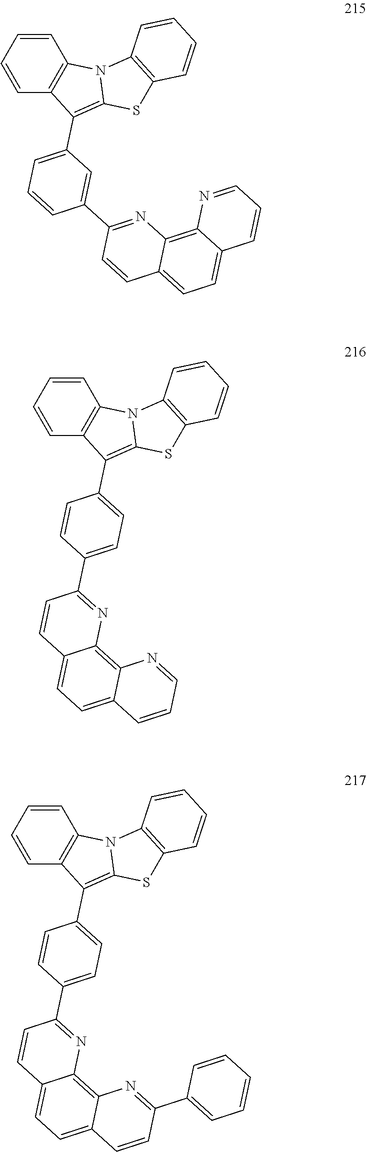 [SMILES: c1cc(-c2ccc3ccc4cccnc4c3n2)cc(-c2c3ccccc3n3c2sc2ccccc23)c1 ; c1cnc2c(c1)ccc1ccc(-c3ccc(-c4c5ccccc5n5c4sc4ccccc45)cc3)nc12 ; c1ccc(-c2ccc3ccc4ccc(-c5ccc(-c6c7ccccc7n7c6sc6ccccc67)cc5)nc4c3n2)cc1]

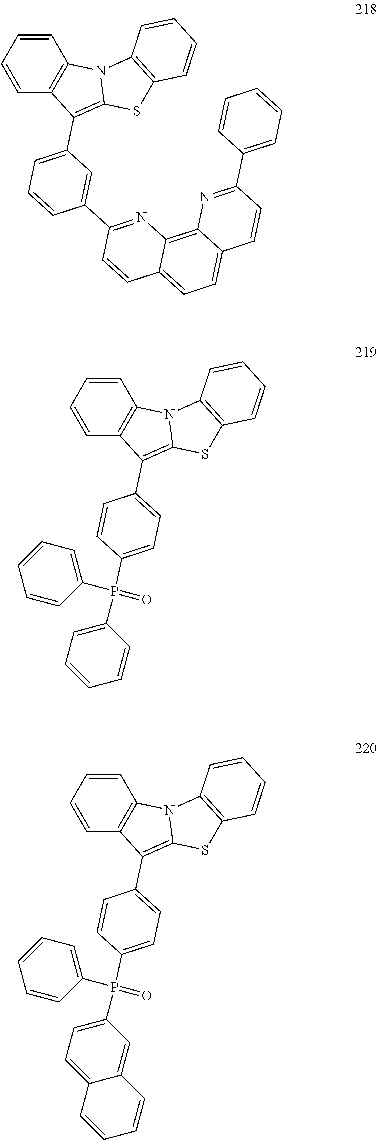 [SMILES: c1ccc(-c2ccc3ccc4ccc(-c5cccc(-c6c7ccccc7n7c6sc6ccccc67)c5)nc4c3n2)cc1 ; O=P(c1ccccc1)(c1ccccc1)c1ccc(-c2c3ccccc3n3c2sc2ccccc23)cc1 ; O=P(c1ccccc1)(c1ccc(-c2c3ccccc3n3c2sc2ccccc23)cc1)c1ccc2ccccc2c1]

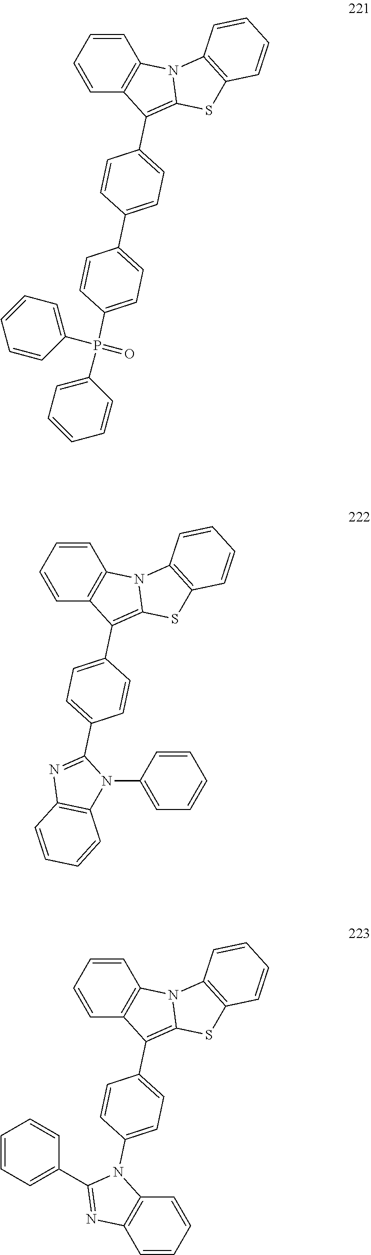 [SMILES: O=P(c1ccccc1)(c1ccccc1)c1ccc(-c2ccc(-c3c4ccccc4n4c3sc3ccccc34)cc2)cc1 ; c1ccc(-n2c(-c3ccc(-c4c5ccccc5n5c4sc4ccccc45)cc3)nc3ccccc32)cc1 ; c1ccc(-c2nc3ccccc3n2-c2ccc(-c3c4ccccc4n4c3sc3ccccc34)cc2)cc1]

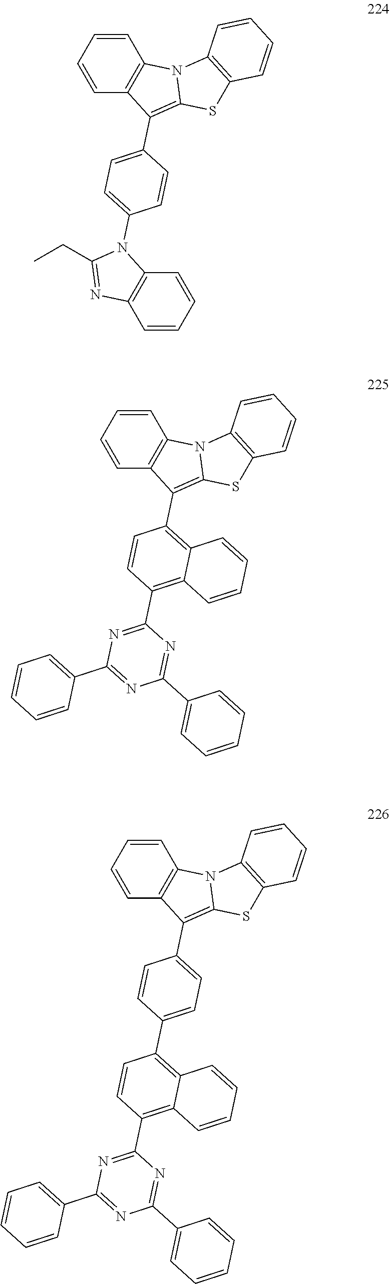 [SMILES: CCc1nc2ccccc2n1-c1ccc(-c2c3ccccc3n3c2sc2ccccc23)cc1 ; c1ccc(-c2nc(-c3ccccc3)nc(-c3ccc(-c4c5ccccc5n5c4sc4ccccc45)c4ccccc34)n2)cc1 ; c1ccc(-c2nc(-c3ccccc3)nc(-c3ccc(-c4ccc(-c5c6ccccc6n6c5sc5ccccc56)cc4)c4ccccc34)n2)cc1]

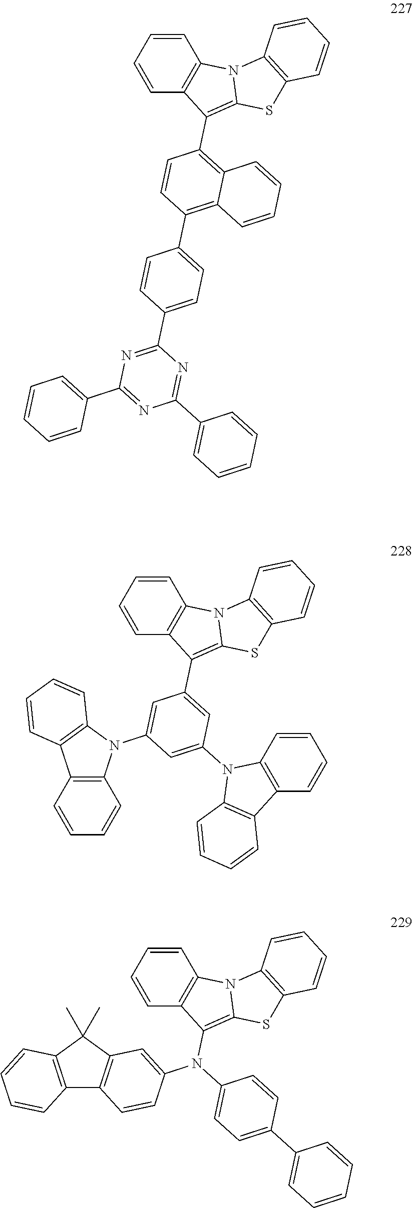 [SMILES: c1ccc(-c2nc(-c3ccccc3)nc(-c3ccc(-c4ccc(-c5c6ccccc6n6c5sc5ccccc56)c5ccccc45)cc3)n2)cc1 ; c1ccc2c(c1)sc1c(-c3cc(-n4c5ccccc5c5ccccc54)cc(-n4c5ccccc5c5ccccc54)c3)c3ccccc3n12 ; CC1(C)c2ccccc2-c2ccc(N(c3ccc(-c4ccccc4)cc3)c3c4ccccc4n4c3sc3ccccc34)cc21]

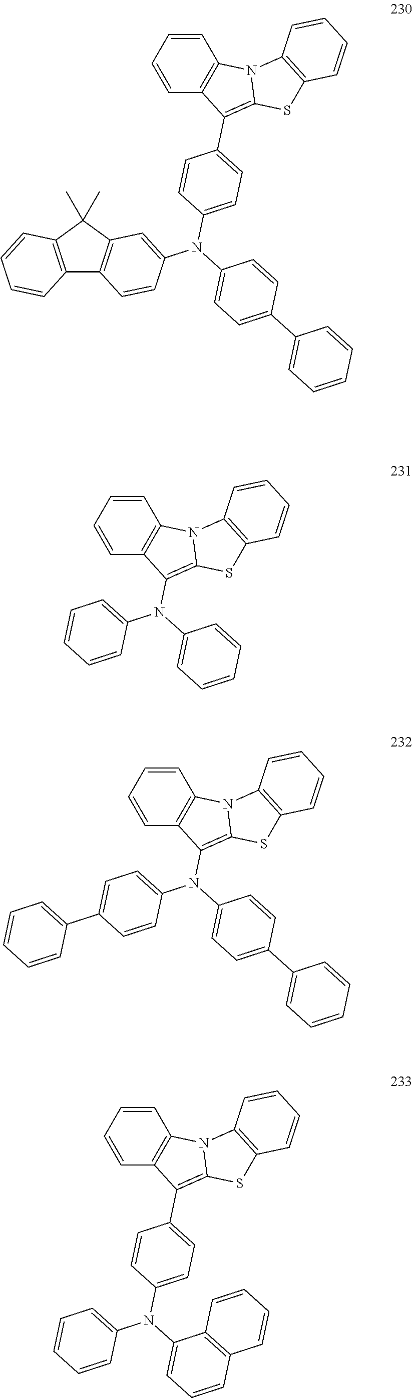 [SMILES: CC1(C)c2ccccc2-c2ccc(N(c3ccc(-c4ccccc4)cc3)c3ccc(-c4c5ccccc5n5c4sc4ccccc45)cc3)cc21 ; c1ccc(N(c2ccccc2)c2c3ccccc3n3c2sc2ccccc23)cc1 ; c1ccc(-c2ccc(N(c3ccc(-c4ccccc4)cc3)c3c4ccccc4n4c3sc3ccccc34)cc2)cc1 ; c1ccc(N(c2ccc(-c3c4ccccc4n4c3sc3ccccc34)cc2)c2cccc3ccccc23)cc1]

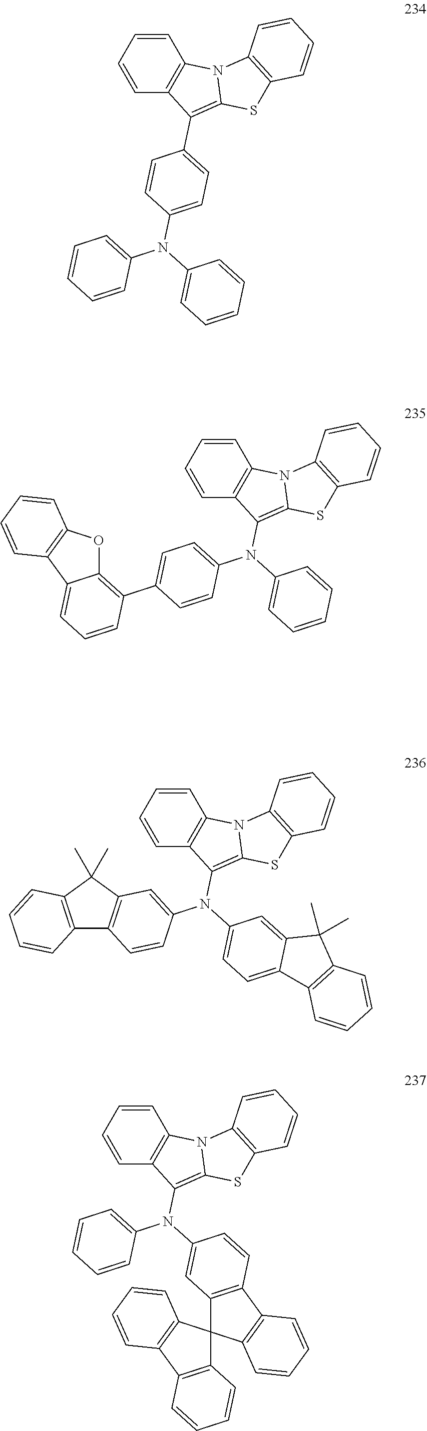 [SMILES: c1ccc(N(c2ccccc2)c2ccc(-c3c4ccccc4n4c3sc3ccccc34)cc2)cc1 ; c1ccc(N(c2ccc(-c3cccc4c3oc3ccccc34)cc2)c2c3ccccc3n3c2sc2ccccc23)cc1 ; CC1(C)c2ccccc2-c2ccc(N(c3ccc4c(c3)C(C)(C)c3ccccc3-4)c3c4ccccc4n4c3sc3ccccc34)cc21 ; c1ccc(N(c2ccc3c(c2)C2(c4ccccc4-c4ccccc42)c2ccccc2-3)c2c3ccccc3n3c2sc2ccccc23)cc1]

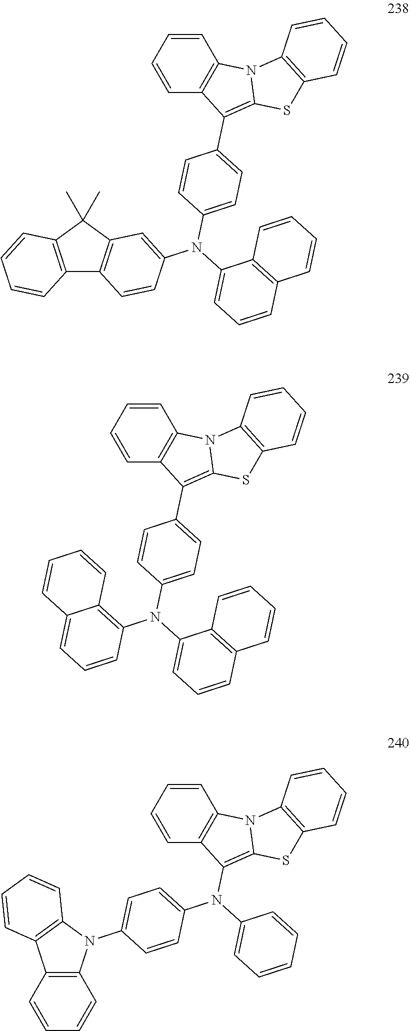 [SMILES: CC1(C)c2ccccc2-c2ccc(N(c3ccc(-c4c5ccccc5n5c4sc4ccccc45)cc3)c3cccc4ccccc34)cc21 ; c1ccc2c(N(c3ccc(-c4c5ccccc5n5c4sc4ccccc45)cc3)c3cccc4ccccc34)cccc2c1 ; c1ccc(N(c2ccc(-n3c4ccccc4c4ccccc43)cc2)c2c3ccccc3n3c2sc2ccccc23)cc1]

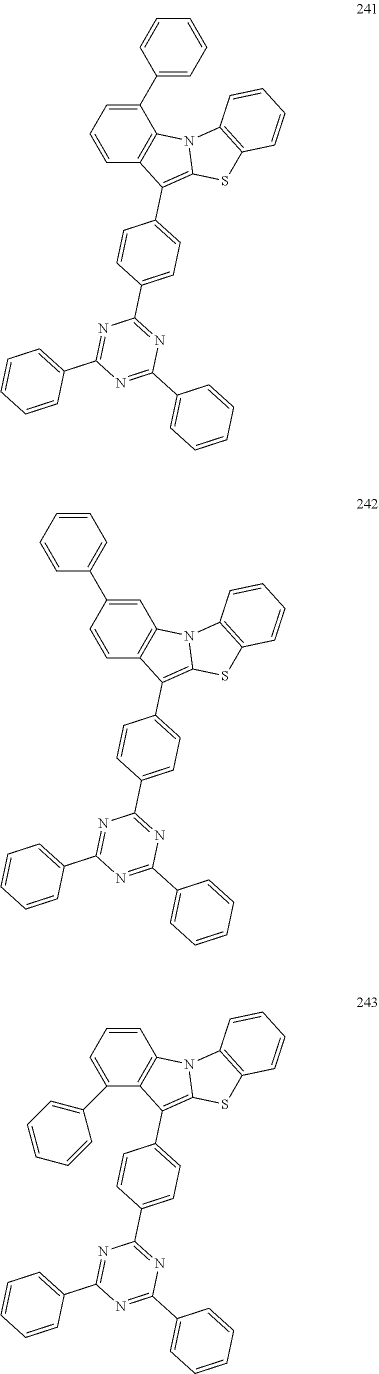 [SMILES: c1ccc(-c2nc(-c3ccccc3)nc(-c3ccc(-c4c5cccc(-c6ccccc6)c5n5c4sc4ccccc45)cc3)n2)cc1 ; c1ccc(-c2ccc3c(-c4ccc(-c5nc(-c6ccccc6)nc(-c6ccccc6)n5)cc4)c4sc5ccccc5n4c3c2)cc1 ; c1ccc(-c2nc(-c3ccccc3)nc(-c3ccc(-c4c5c(-c6ccccc6)cccc5n5c4sc4ccccc45)cc3)n2)cc1]

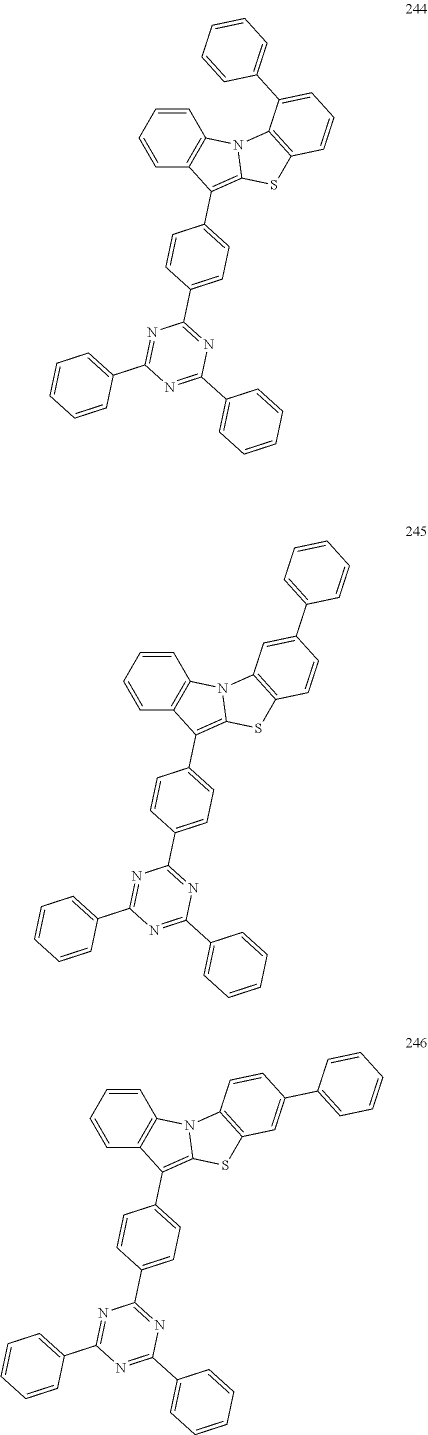 [SMILES: c1ccc(-c2nc(-c3ccccc3)nc(-c3ccc(-c4c5ccccc5n5c4sc4cccc(-c6ccccc6)c45)cc3)n2)cc1 ; c1ccc(-c2ccc3sc4c(-c5ccc(-c6nc(-c7ccccc7)nc(-c7ccccc7)n6)cc5)c5ccccc5n4c3c2)cc1 ; c1ccc(-c2ccc3c(c2)sc2c(-c4ccc(-c5nc(-c6ccccc6)nc(-c6ccccc6)n5)cc4)c4ccccc4n23)cc1]

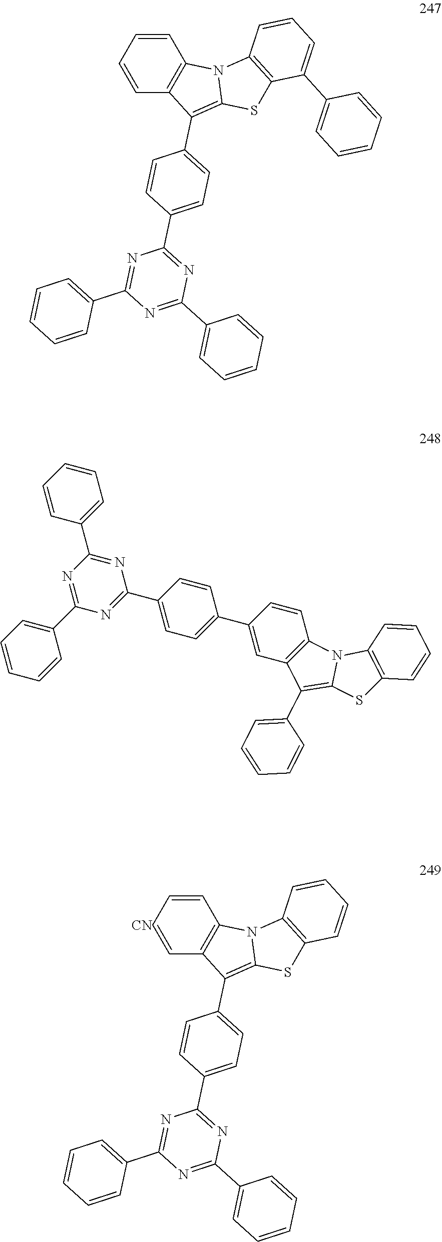

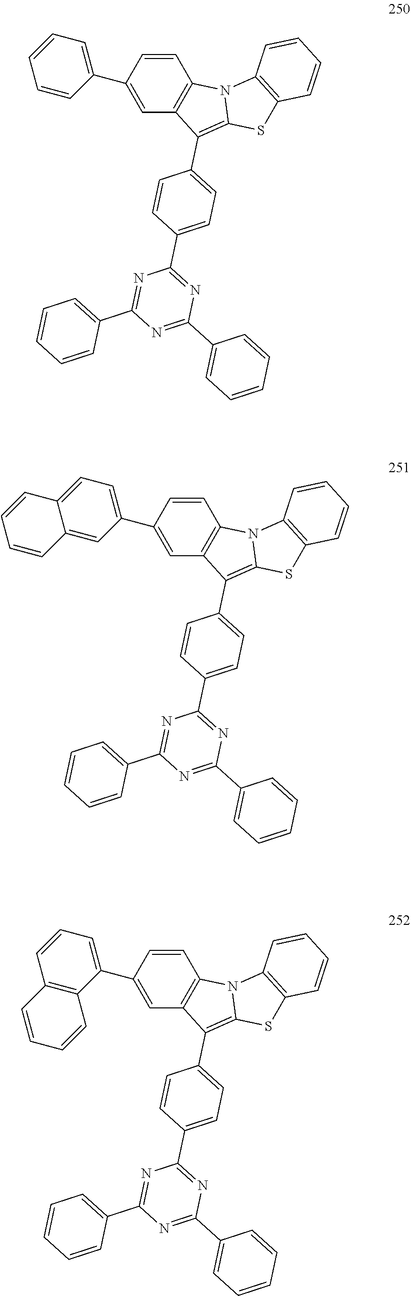 [SMILES: c1ccc(-c2ccc3c(c2)c(-c2ccc(-c4nc(-c5ccccc5)nc(-c5ccccc5)n4)cc2)c2sc4ccccc4n23)cc1 ; c1ccc(-c2nc(-c3ccccc3)nc(-c3ccc(-c4c5cc(-c6ccc7ccccc7c6)ccc5n5c4sc4ccccc45)cc3)n2)cc1 ; c1ccc(-c2nc(-c3ccccc3)nc(-c3ccc(-c4c5cc(-c6cccc7ccccc67)ccc5n5c4sc4ccccc45)cc3)n2)cc1]

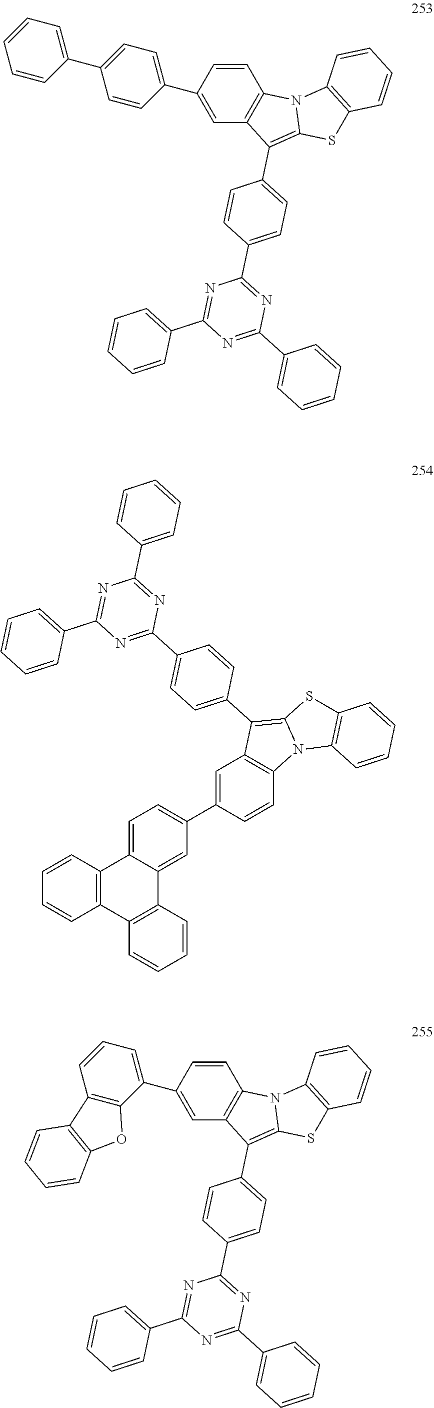 [SMILES: c1ccc(-c2ccc(-c3ccc4c(c3)c(-c3ccc(-c5nc(-c6ccccc6)nc(-c6ccccc6)n5)cc3)c3sc5ccccc5n34)cc2)cc1 ; c1ccc(-c2nc(-c3ccccc3)nc(-c3ccc(-c4c5cc(-c6ccc7c8ccccc8c8ccccc8c7c6)ccc5n5c4sc4ccccc45)cc3)n2)cc1 ; c1ccc(-c2nc(-c3ccccc3)nc(-c3ccc(-c4c5cc(-c6cccc7c6oc6ccccc67)ccc5n5c4sc4ccccc45)cc3)n2)cc1]

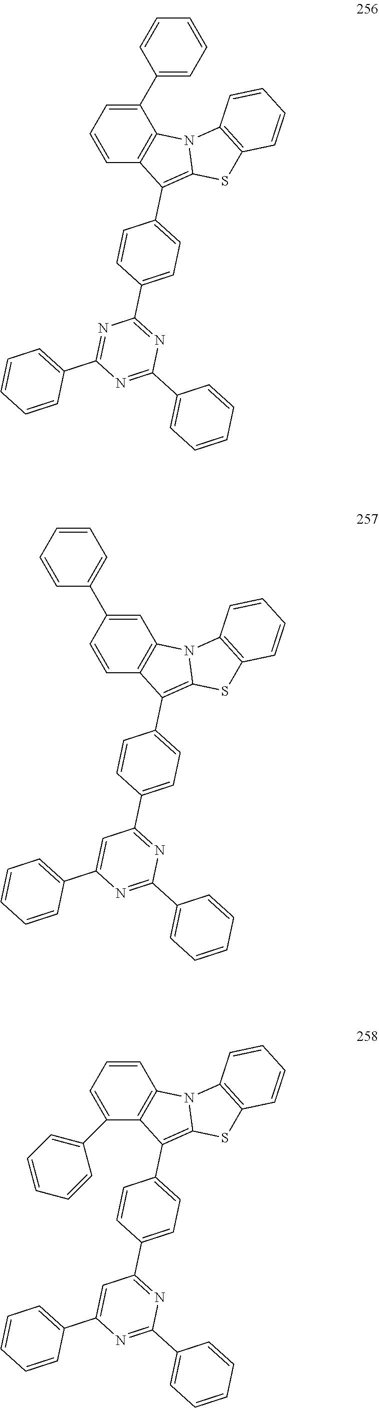 [SMILES: c1ccc(-c2nc(-c3ccccc3)nc(-c3ccc(-c4c5cccc(-c6ccccc6)c5n5c4sc4ccccc45)cc3)n2)cc1 ; c1ccc(-c2ccc3c(-c4ccc(-c5cc(-c6ccccc6)nc(-c6ccccc6)n5)cc4)c4sc5ccccc5n4c3c2)cc1 ; c1ccc(-c2cc(-c3ccc(-c4c5c(-c6ccccc6)cccc5n5c4sc4ccccc45)cc3)nc(-c3ccccc3)n2)cc1]

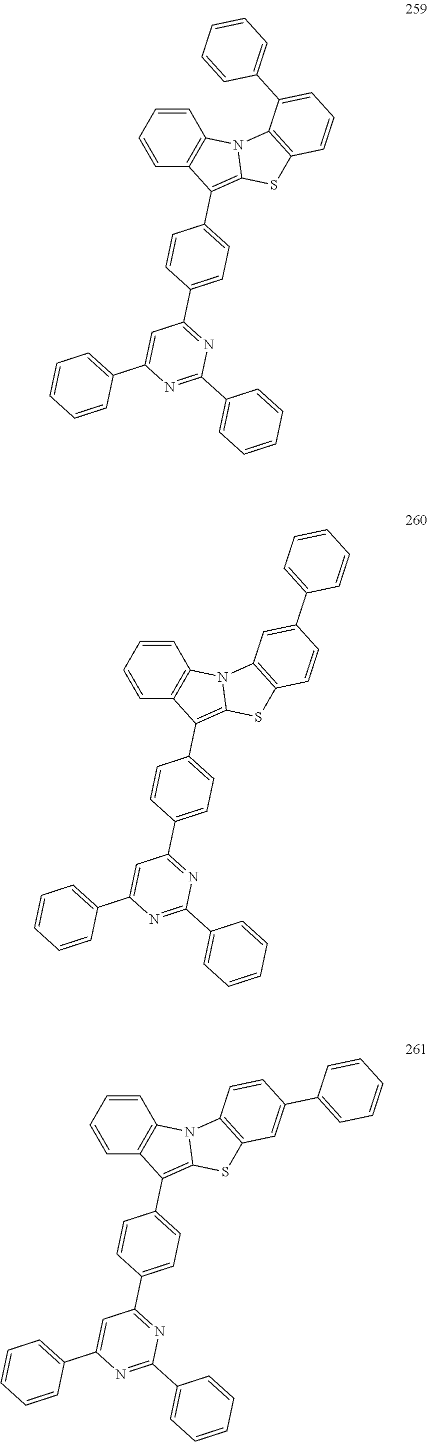 [SMILES: c1ccc(-c2cc(-c3ccc(-c4c5ccccc5n5c4sc4cccc(-c6ccccc6)c45)cc3)nc(-c3ccccc3)n2)cc1 ; c1ccc(-c2ccc3sc4c(-c5ccc(-c6cc(-c7ccccc7)nc(-c7ccccc7)n6)cc5)c5ccccc5n4c3c2)cc1 ; c1ccc(-c2ccc3c(c2)sc2c(-c4ccc(-c5cc(-c6ccccc6)nc(-c6ccccc6)n5)cc4)c4ccccc4n23)cc1]

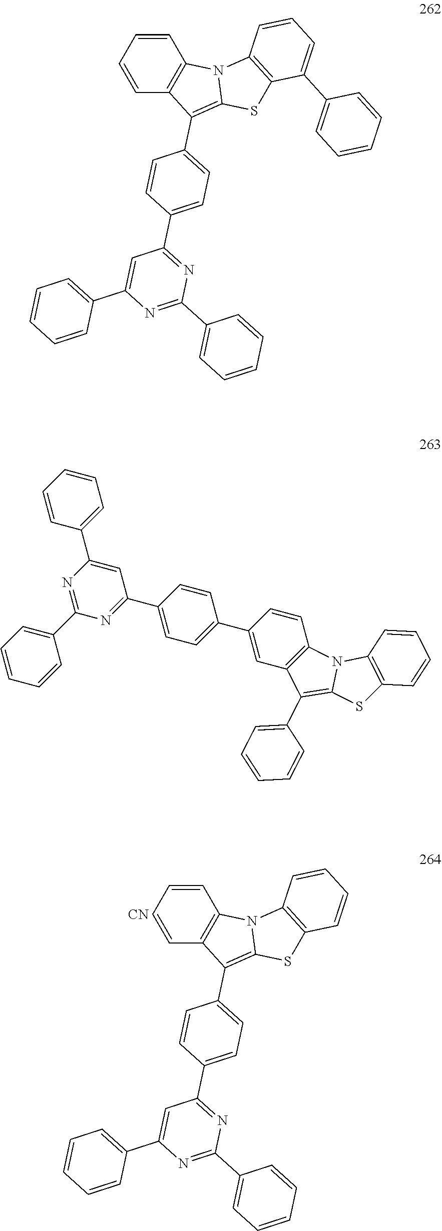

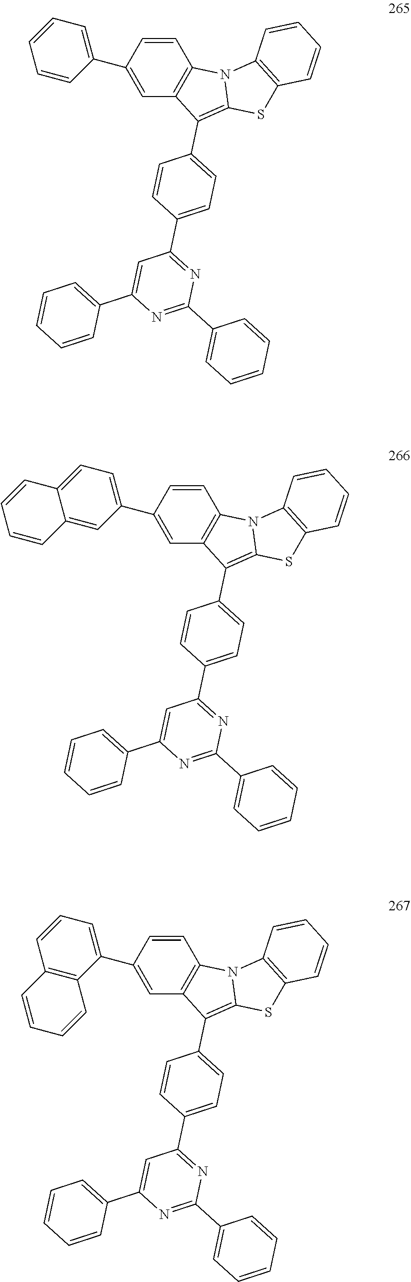 [SMILES: c1ccc(-c2ccc3c(c2)c(-c2ccc(-c4cc(-c5ccccc5)nc(-c5ccccc5)n4)cc2)c2sc4ccccc4n23)cc1 ; c1ccc(-c2cc(-c3ccc(-c4c5cc(-c6ccc7ccccc7c6)ccc5n5c4sc4ccccc45)cc3)nc(-c3ccccc3)n2)cc1 ; c1ccc(-c2cc(-c3ccc(-c4c5cc(-c6cccc7ccccc67)ccc5n5c4sc4ccccc45)cc3)nc(-c3ccccc3)n2)cc1]

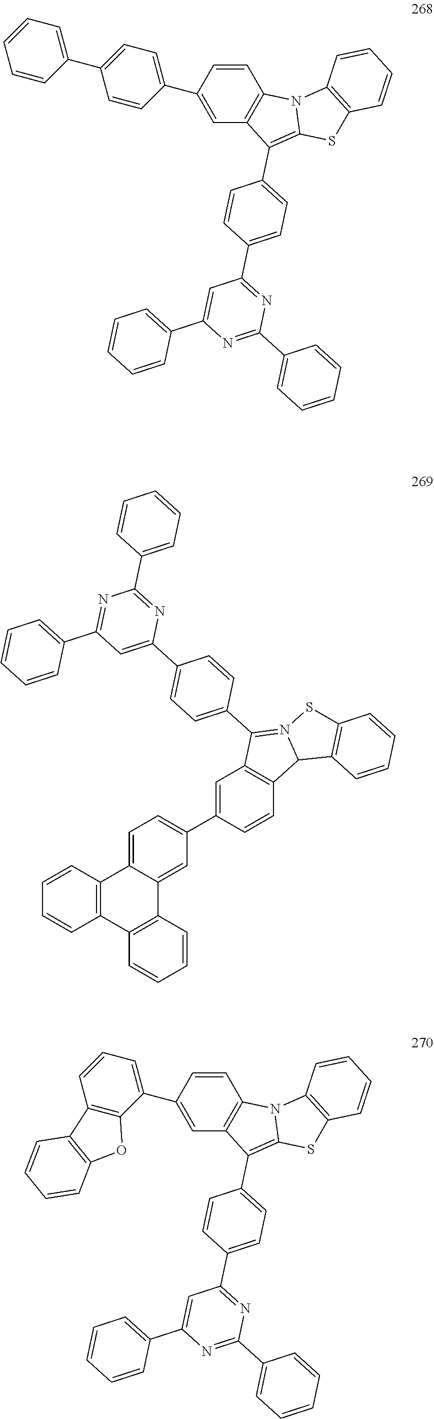

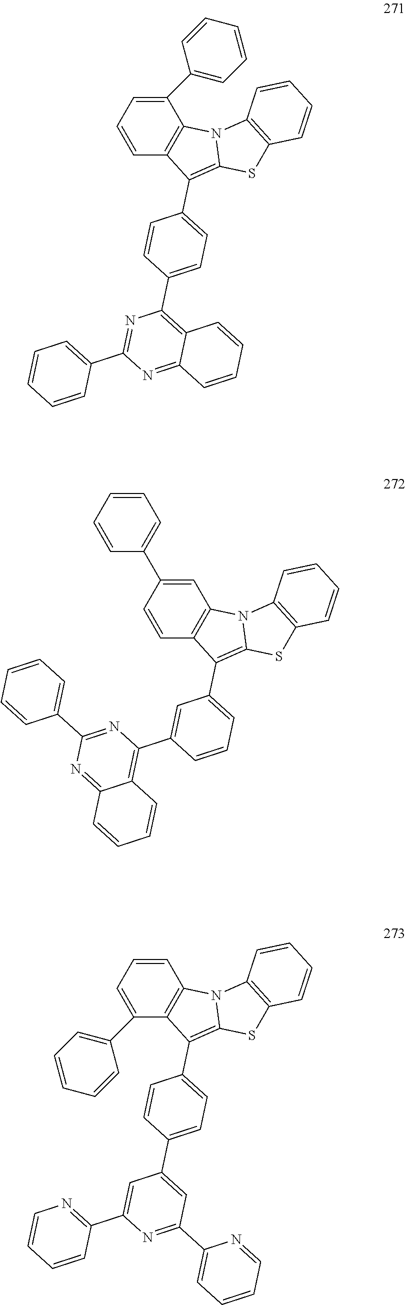 [SMILES: c1ccc(-c2nc(-c3ccc(-c4c5cccc(-c6ccccc6)c5n5c4sc4ccccc45)cc3)c3ccccc3n2)cc1 ; c1ccc(-c2ccc3c(-c4cccc(-c5nc(-c6ccccc6)nc6ccccc56)c4)c4sc5ccccc5n4c3c2)cc1 ; c1ccc(-c2cccc3c2c(-c2ccc(-c4cc(-c5ccccn5)nc(-c5ccccn5)c4)cc2)c2sc4ccccc4n23)cc1]

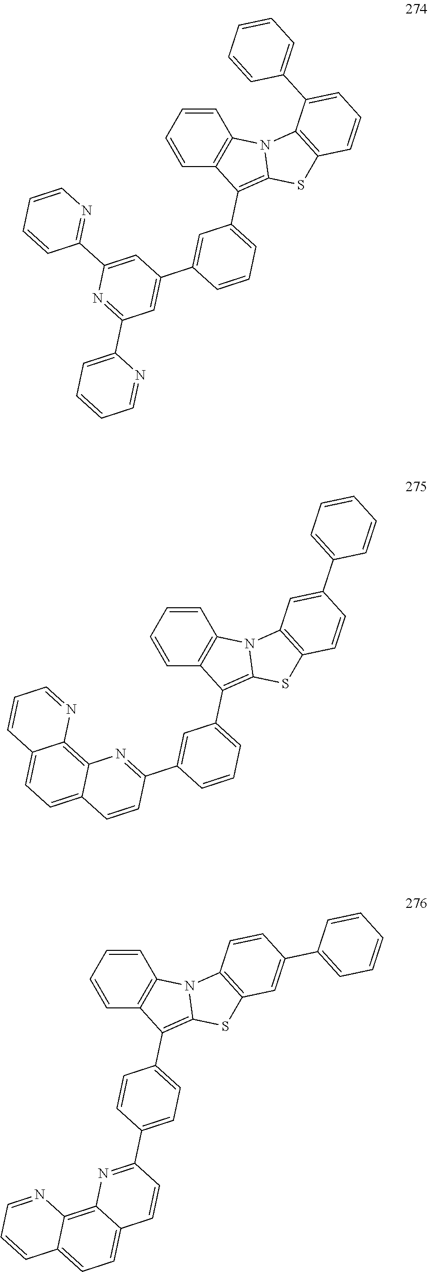 [SMILES: c1ccc(-c2cccc3sc4c(-c5cccc(-c6cc(-c7ccccn7)nc(-c7ccccn7)c6)c5)c5ccccc5n4c23)cc1 ; c1ccc(-c2ccc3sc4c(-c5cccc(-c6ccc7ccc8cccnc8c7n6)c5)c5ccccc5n4c3c2)cc1 ; c1ccc(-c2ccc3c(c2)sc2c(-c4ccc(-c5ccc6ccc7cccnc7c6n5)cc4)c4ccccc4n23)cc1]

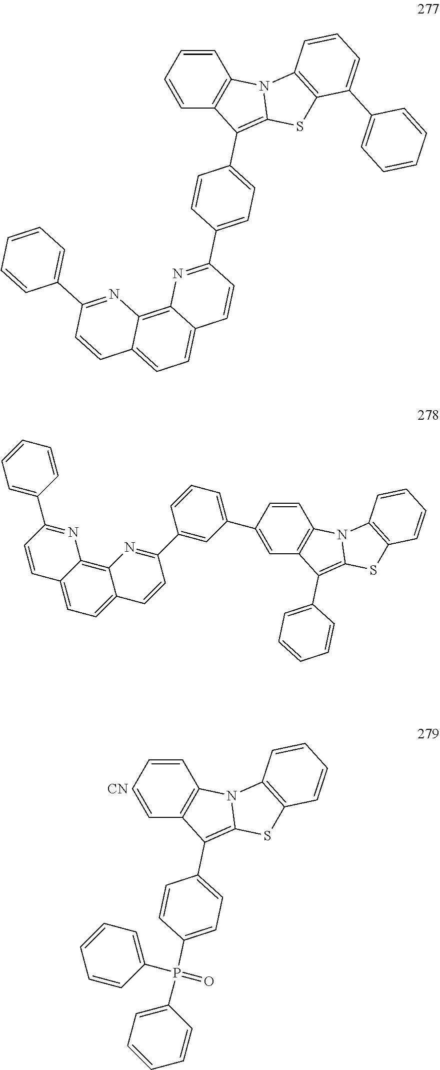

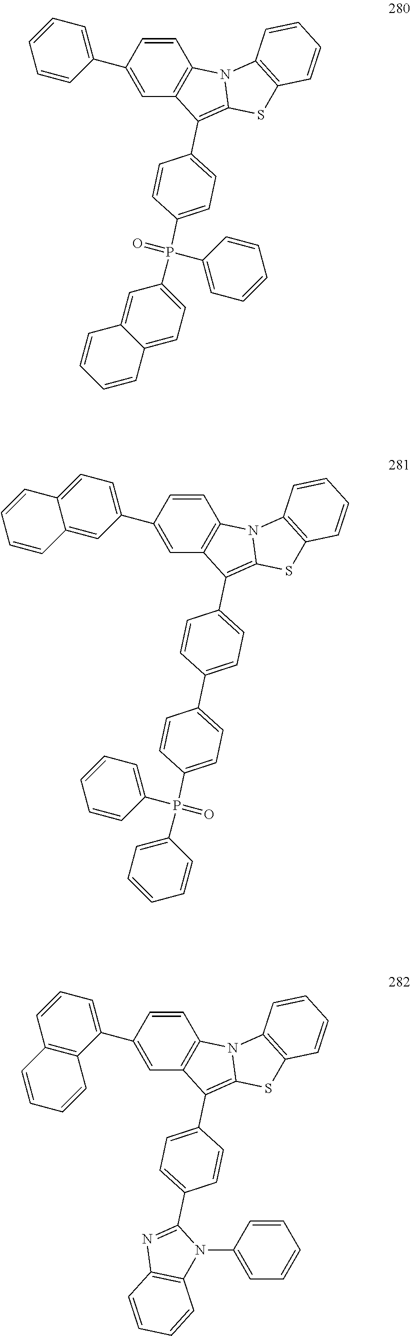 [SMILES: O=P(c1ccccc1)(c1ccc(-c2c3cc(-c4ccccc4)ccc3n3c2sc2ccccc23)cc1)c1ccc2ccccc2c1 ; O=P(c1ccccc1)(c1ccccc1)c1ccc(-c2ccc(-c3c4cc(-c5ccc6ccccc6c5)ccc4n4c3sc3ccccc34)cc2)cc1 ; c1ccc(-n2c(-c3ccc(-c4c5cc(-c6cccc7ccccc67)ccc5n5c4sc4ccccc45)cc3)nc3ccccc32)cc1]

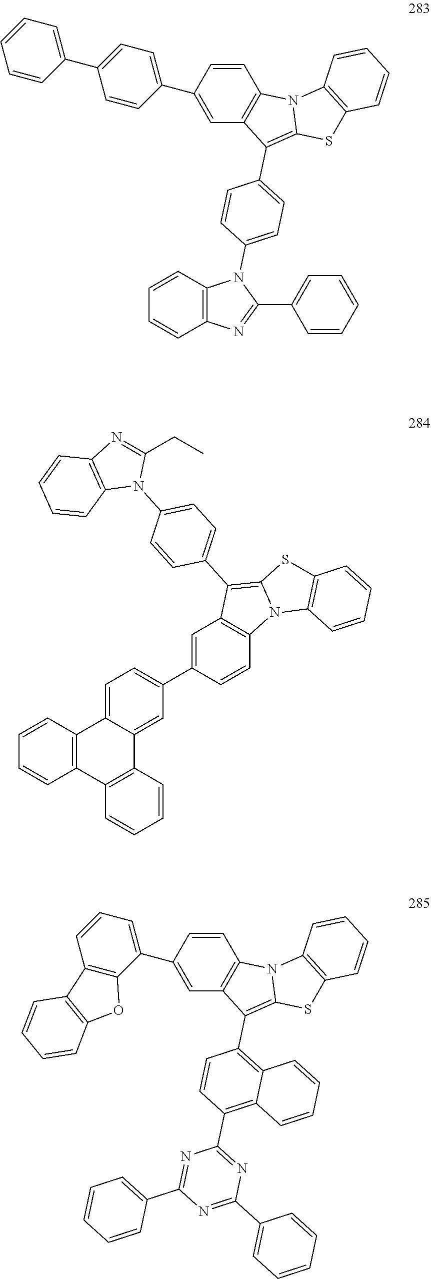 [SMILES: c1ccc(-c2ccc(-c3ccc4c(c3)c(-c3ccc(-n5c(-c6ccccc6)nc6ccccc65)cc3)c3sc5ccccc5n34)cc2)cc1 ; CCc1nc2ccccc2n1-c1ccc(-c2c3cc(-c4ccc5c6ccccc6c6ccccc6c5c4)ccc3n3c2sc2ccccc23)cc1 ; c1ccc(-c2nc(-c3ccccc3)nc(-c3ccc(-c4c5cc(-c6cccc7c6oc6ccccc67)ccc5n5c4sc4ccccc45)c4ccccc34)n2)cc1]

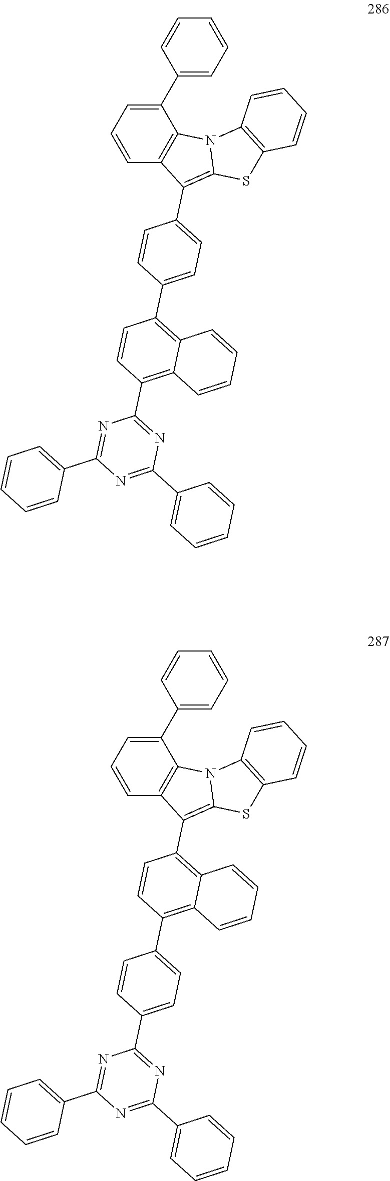 [SMILES: c1ccc(-c2nc(-c3ccccc3)nc(-c3ccc(-c4ccc(-c5c6cccc(-c7ccccc7)c6n6c5sc5ccccc56)cc4)c4ccccc34)n2)cc1 ; c1ccc(-c2nc(-c3ccccc3)nc(-c3ccc(-c4ccc(-c5c6cccc(-c7ccccc7)c6n6c5sc5ccccc56)c5ccccc45)cc3)n2)cc1]

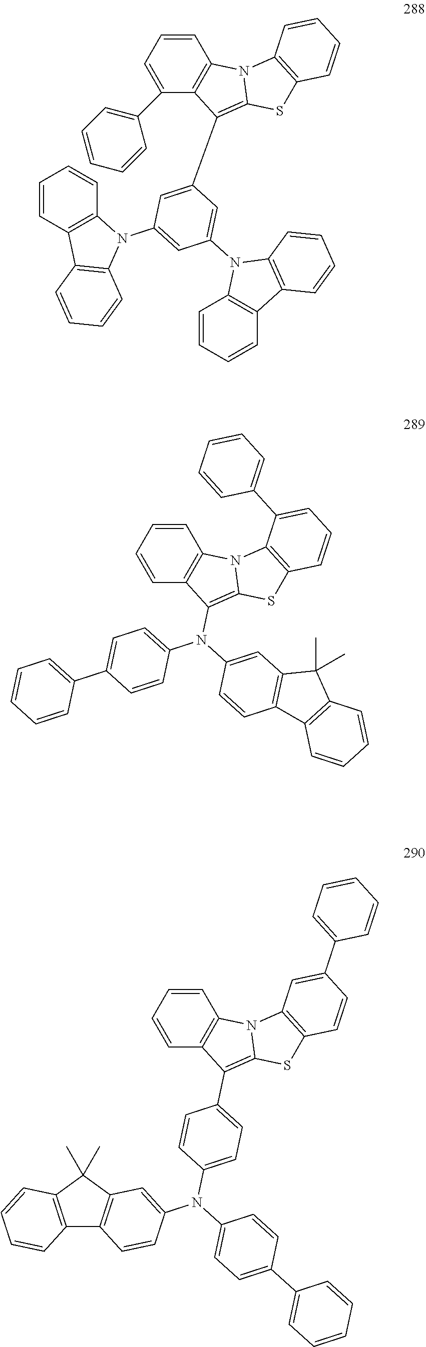 [SMILES: c1ccc(-c2cccc3c2c(-c2cc(-n4c5ccccc5c5ccccc54)cc(-n4c5ccccc5c5ccccc54)c2)c2sc4ccccc4n23)cc1 ; CC1(C)c2ccccc2-c2ccc(N(c3ccc(-c4ccccc4)cc3)c3c4ccccc4n4c3sc3cccc(-c5ccccc5)c34)cc21 ; CC1(C)c2ccccc2-c2ccc(N(c3ccc(-c4ccccc4)cc3)c3ccc(-c4c5ccccc5n5c4sc4ccc(-c6ccccc6)cc45)cc3)cc21]

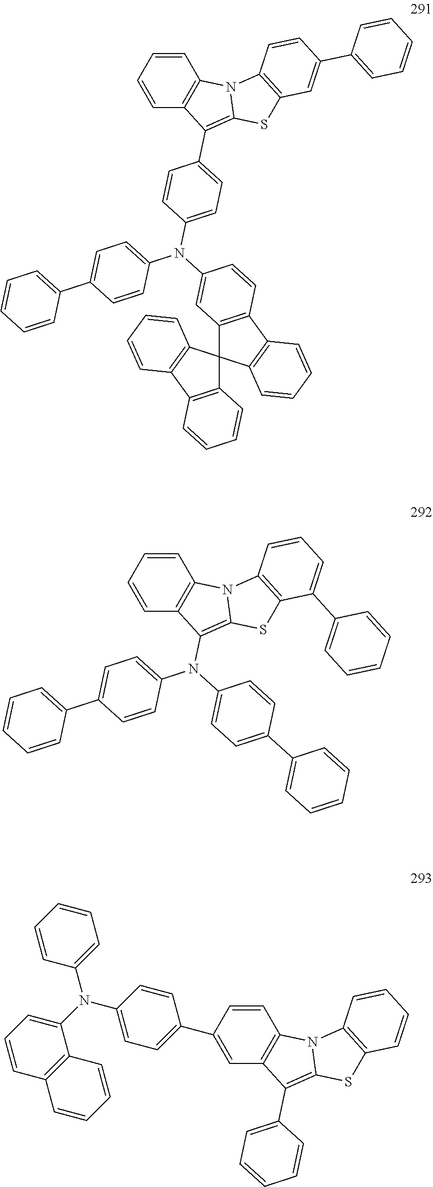 [SMILES: c1ccc(-c2ccc(N(c3ccc(-c4c5ccccc5n5c4sc4cc(-c6ccccc6)ccc45)cc3)c3ccc4c(c3)C3(c5ccccc5-c5ccccc53)c3ccccc3-4)cc2)cc1 ; c1ccc(-c2ccc(N(c3ccc(-c4ccccc4)cc3)c3c4ccccc4n4c3sc3c(-c5ccccc5)cccc34)cc2)cc1 ; c1ccc(-c2c3cc(-c4ccc(N(c5ccccc5)c5cccc6ccccc56)cc4)ccc3n3c2sc2ccccc23)cc1]

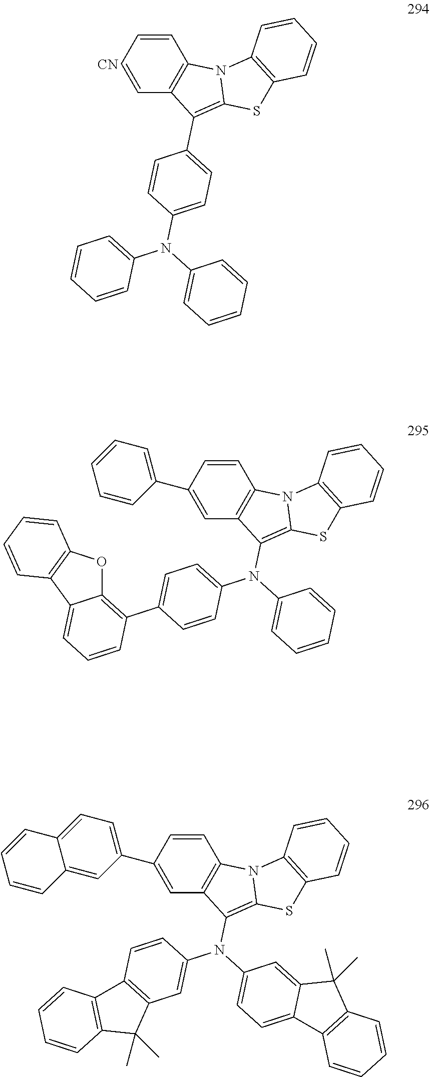

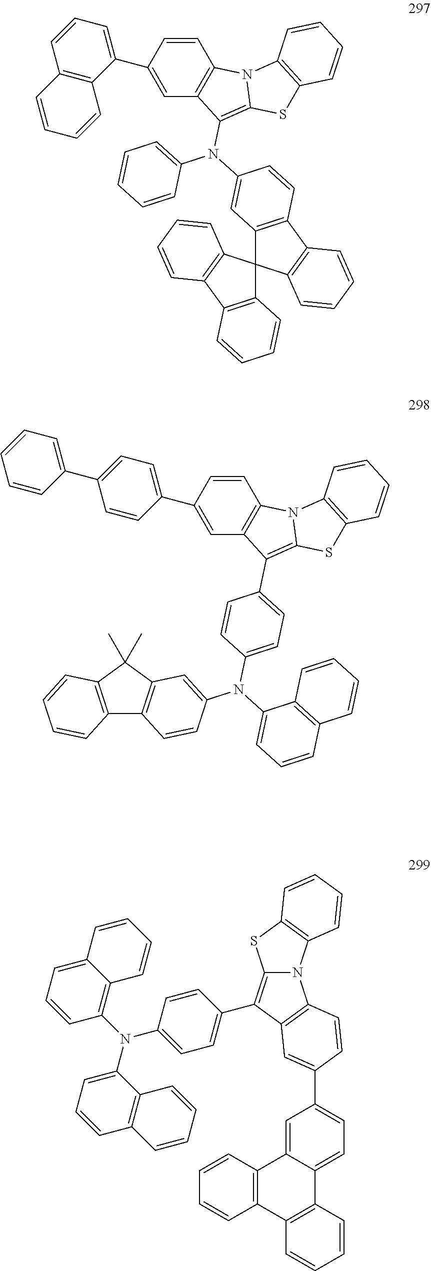 [SMILES: c1ccc(N(c2ccc3c(c2)C2(c4ccccc4-c4ccccc42)c2ccccc2-3)c2c3cc(-c4cccc5ccccc45)ccc3n3c2sc2ccccc23)cc1 ; CC1(C)c2ccccc2-c2ccc(N(c3ccc(-c4c5cc(-c6ccc(-c7ccccc7)cc6)ccc5n5c4sc4ccccc45)cc3)c3cccc4ccccc34)cc21 ; c1ccc2c(N(c3ccc(-c4c5cc(-c6ccc7c8ccccc8c8ccccc8c7c6)ccc5n5c4sc4ccccc45)cc3)c3cccc4ccccc34)cccc2c1]

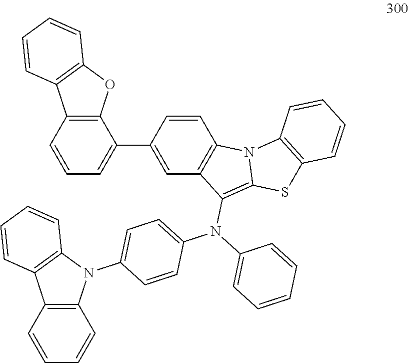 [SMILES: c1ccc(N(c2ccc(-n3c4ccccc4c4ccccc43)cc2)c2c3cc(-c4cccc5c4oc4ccccc45)ccc3n3c2sc2ccccc23)cc1]

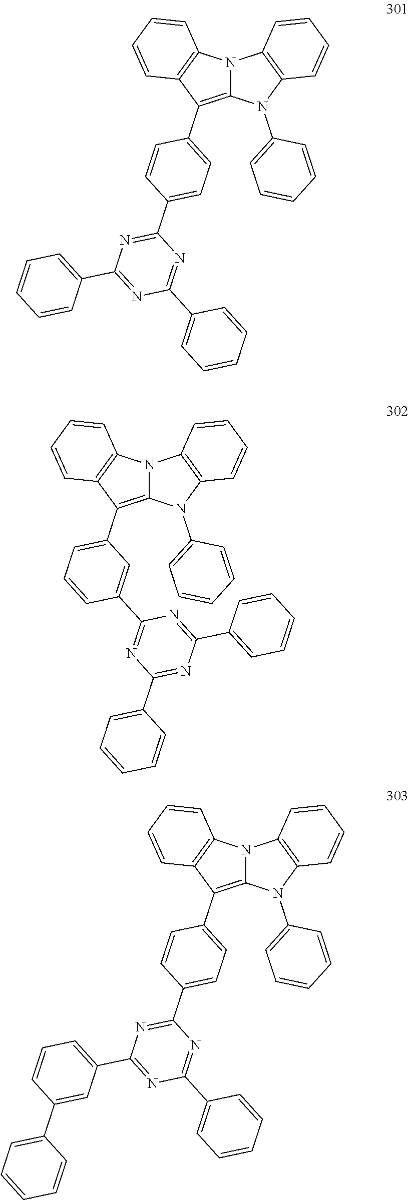

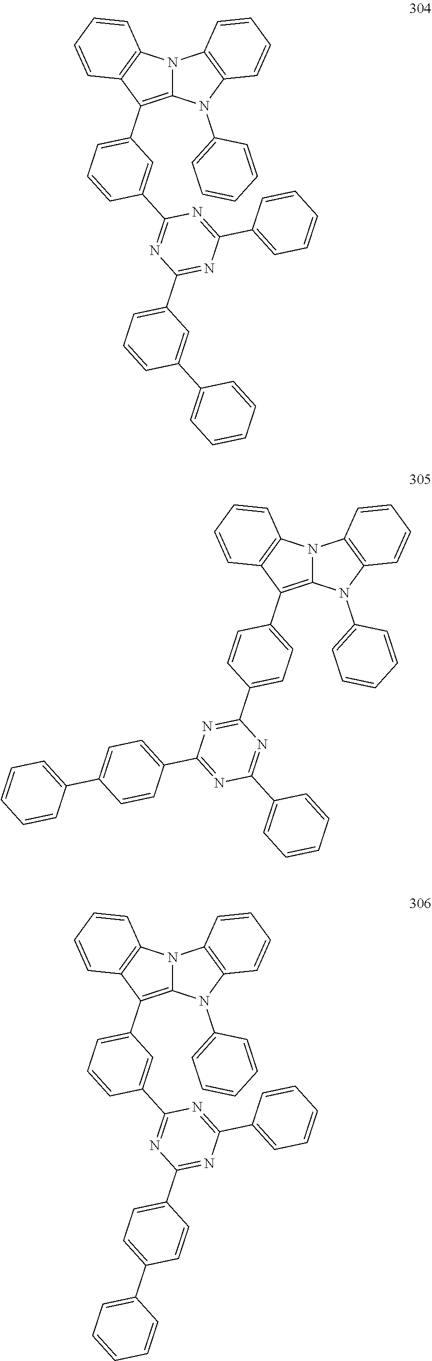 [SMILES: c1ccc(-c2cccc(-c3nc(-c4ccccc4)nc(-c4cccc(-c5c6ccccc6n6c7ccccc7n(-c7ccccc7)c56)c4)n3)c2)cc1 ; c1ccc(-c2ccc(-c3nc(-c4ccccc4)nc(-c4ccc(-c5c6ccccc6n6c7ccccc7n(-c7ccccc7)c56)cc4)n3)cc2)cc1 ; c1ccc(-c2ccc(-c3nc(-c4ccccc4)nc(-c4cccc(-c5c6ccccc6n6c7ccccc7n(-c7ccccc7)c56)c4)n3)cc2)cc1]

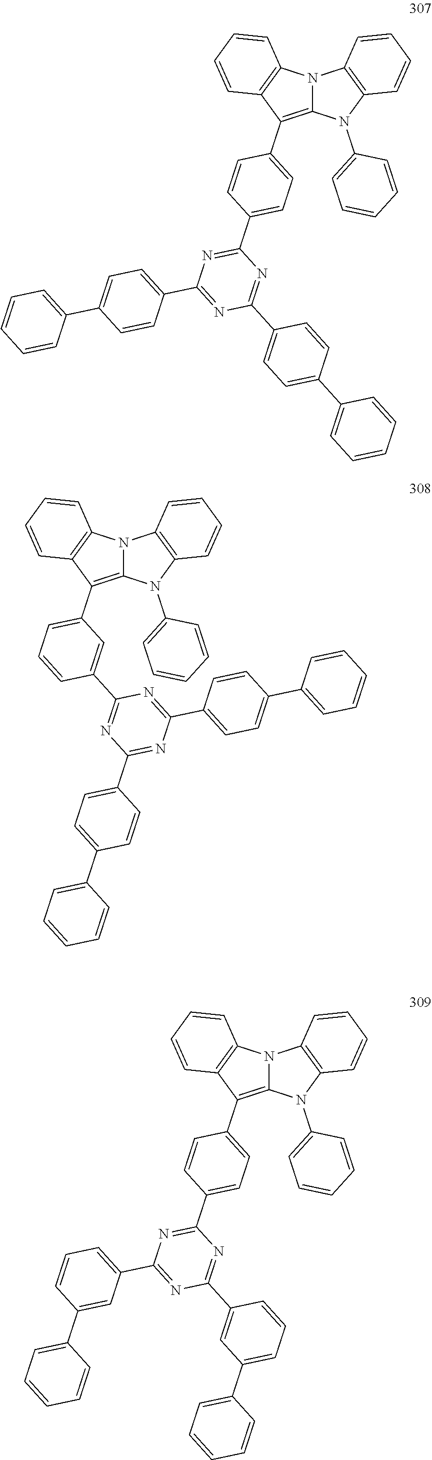 [SMILES: c1ccc(-c2ccc(-c3nc(-c4ccc(-c5ccccc5)cc4)nc(-c4ccc(-c5c6ccccc6n6c7ccccc7n(-c7ccccc7)c56)cc4)n3)cc2)cc1 ; c1ccc(-c2ccc(-c3nc(-c4ccc(-c5ccccc5)cc4)nc(-c4cccc(-c5c6ccccc6n6c7ccccc7n(-c7ccccc7)c56)c4)n3)cc2)cc1 ; c1ccc(-c2cccc(-c3nc(-c4ccc(-c5c6ccccc6n6c7ccccc7n(-c7ccccc7)c56)cc4)nc(-c4cccc(-c5ccccc5)c4)n3)c2)cc1]

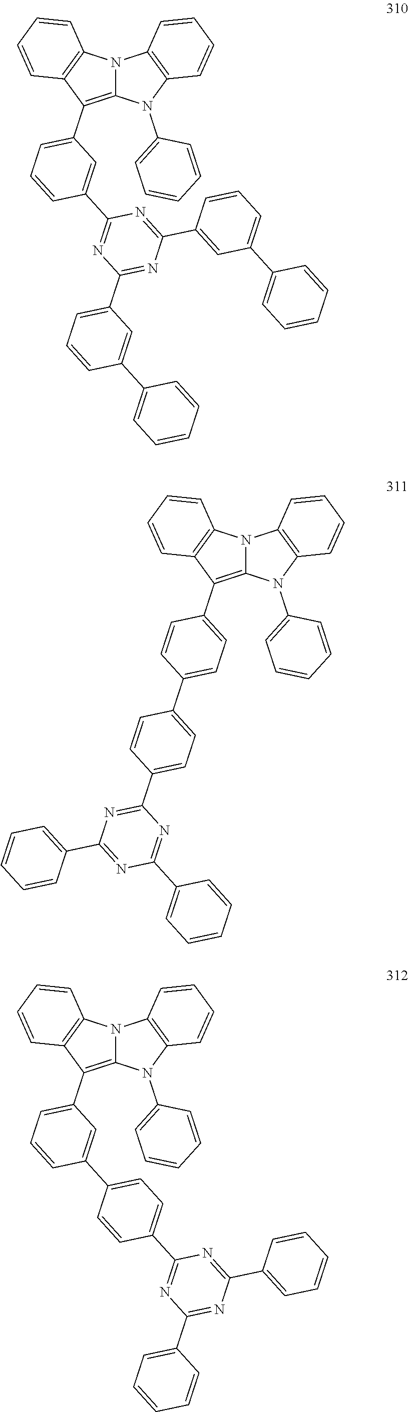

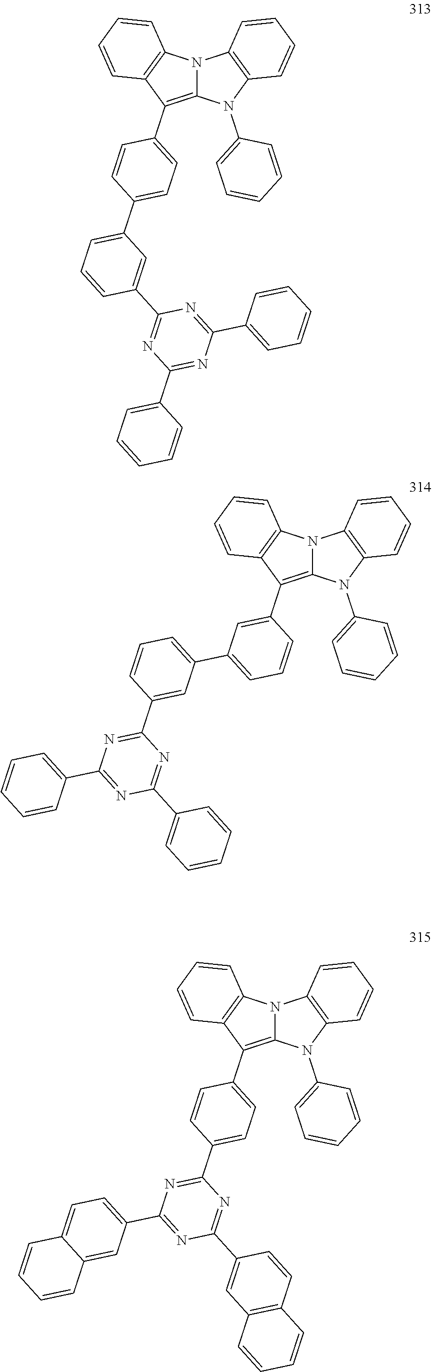 [SMILES: c1ccc(-c2nc(-c3ccccc3)nc(-c3cccc(-c4ccc(-c5c6ccccc6n6c7ccccc7n(-c7ccccc7)c56)cc4)c3)n2)cc1 ; c1ccc(-c2nc(-c3ccccc3)nc(-c3cccc(-c4cccc(-c5c6ccccc6n6c7ccccc7n(-c7ccccc7)c56)c4)c3)n2)cc1 ; c1ccc(-n2c3ccccc3n3c4ccccc4c(-c4ccc(-c5nc(-c6ccc7ccccc7c6)nc(-c6ccc7ccccc7c6)n5)cc4)c23)cc1]

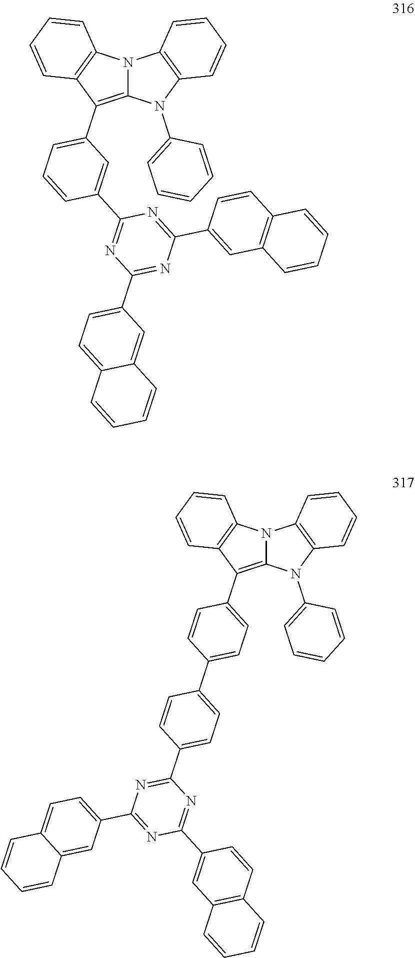 [SMILES: c1ccc(-n2c3ccccc3n3c4ccccc4c(-c4cccc(-c5nc(-c6ccc7ccccc7c6)nc(-c6ccc7ccccc7c6)n5)c4)c23)cc1 ; c1ccc(-n2c3ccccc3n3c4ccccc4c(-c4ccc(-c5ccc(-c6nc(-c7ccc8ccccc8c7)nc(-c7ccc8ccccc8c7)n6)cc5)cc4)c23)cc1]

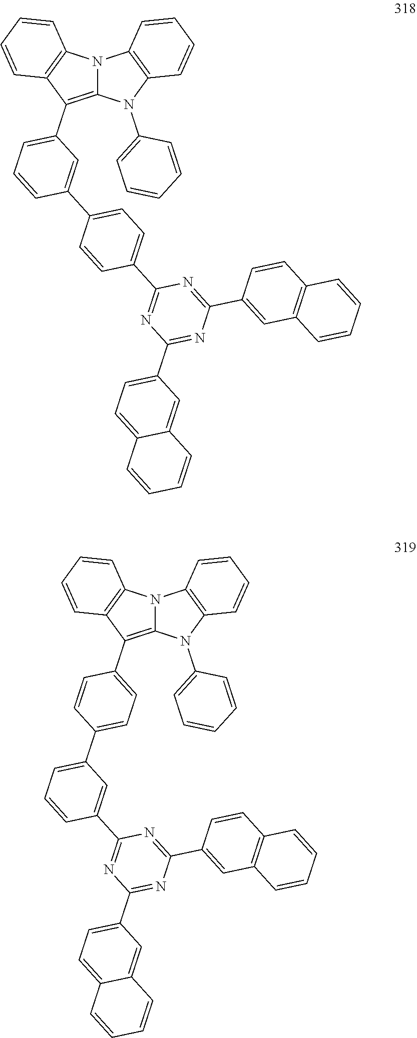 [SMILES: c1ccc(-n2c3ccccc3n3c4ccccc4c(-c4cccc(-c5ccc(-c6nc(-c7ccc8ccccc8c7)nc(-c7ccc8ccccc8c7)n6)cc5)c4)c23)cc1 ; c1ccc(-n2c3ccccc3n3c4ccccc4c(-c4ccc(-c5cccc(-c6nc(-c7ccc8ccccc8c7)nc(-c7ccc8ccccc8c7)n6)c5)cc4)c23)cc1]

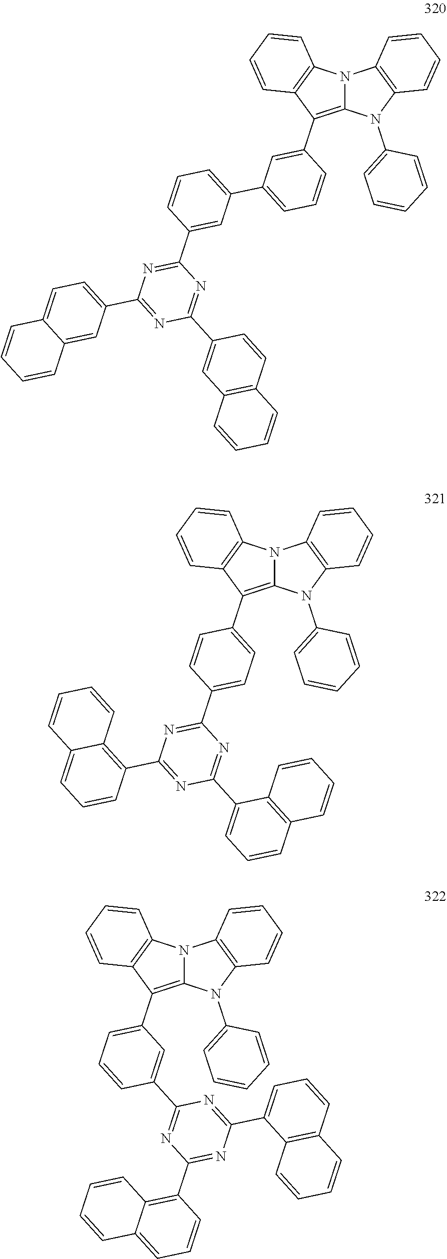 [SMILES: c1ccc(-n2c3ccccc3n3c4ccccc4c(-c4cccc(-c5cccc(-c6nc(-c7ccc8ccccc8c7)nc(-c7ccc8ccccc8c7)n6)c5)c4)c23)cc1 ; c1ccc(-n2c3ccccc3n3c4ccccc4c(-c4ccc(-c5nc(-c6cccc7ccccc67)nc(-c6cccc7ccccc67)n5)cc4)c23)cc1 ; c1ccc(-n2c3ccccc3n3c4ccccc4c(-c4cccc(-c5nc(-c6cccc7ccccc67)nc(-c6cccc7ccccc67)n5)c4)c23)cc1]

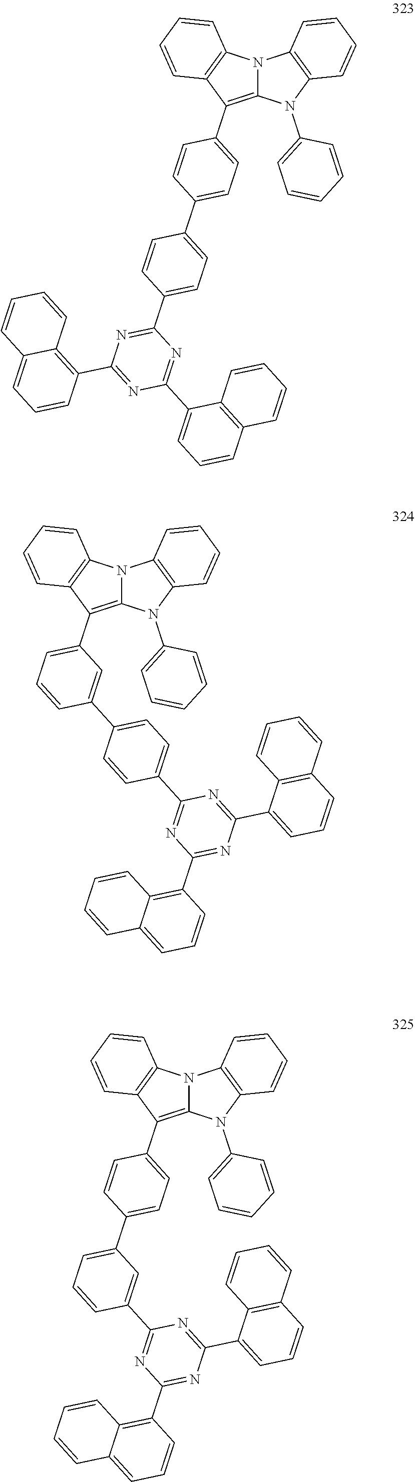 [SMILES: c1ccc(-n2c3ccccc3n3c4ccccc4c(-c4ccc(-c5ccc(-c6nc(-c7cccc8ccccc78)nc(-c7cccc8ccccc78)n6)cc5)cc4)c23)cc1 ; c1ccc(-n2c3ccccc3n3c4ccccc4c(-c4cccc(-c5ccc(-c6nc(-c7cccc8ccccc78)nc(-c7cccc8ccccc78)n6)cc5)c4)c23)cc1 ; c1ccc(-n2c3ccccc3n3c4ccccc4c(-c4ccc(-c5cccc(-c6nc(-c7cccc8ccccc78)nc(-c7cccc8ccccc78)n6)c5)cc4)c23)cc1]

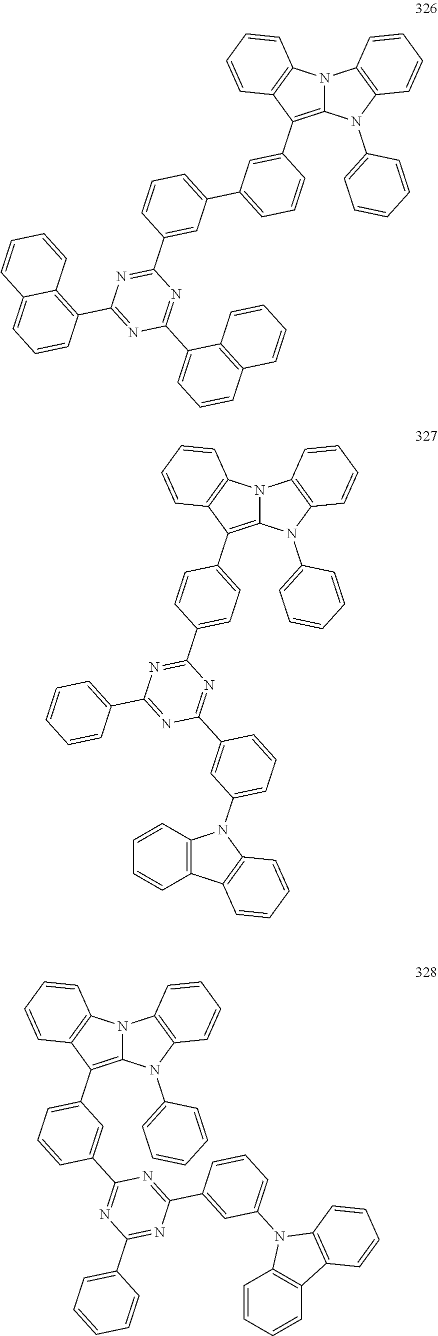

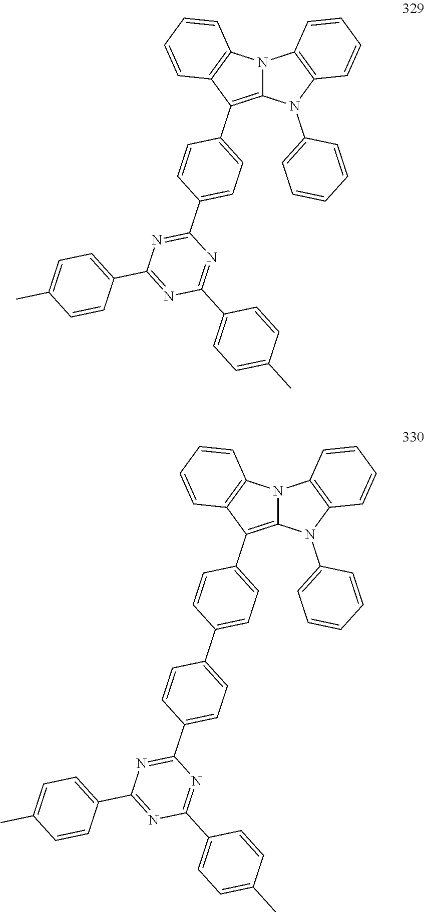 [SMILES: Cc1ccc(-c2nc(-c3ccc(C)cc3)nc(-c3ccc(-c4c5ccccc5n5c6ccccc6n(-c6ccccc6)c45)cc3)n2)cc1 ; Cc1ccc(-c2nc(-c3ccc(C)cc3)nc(-c3ccc(-c4ccc(-c5c6ccccc6n6c7ccccc7n(-c7ccccc7)c56)cc4)cc3)n2)cc1]

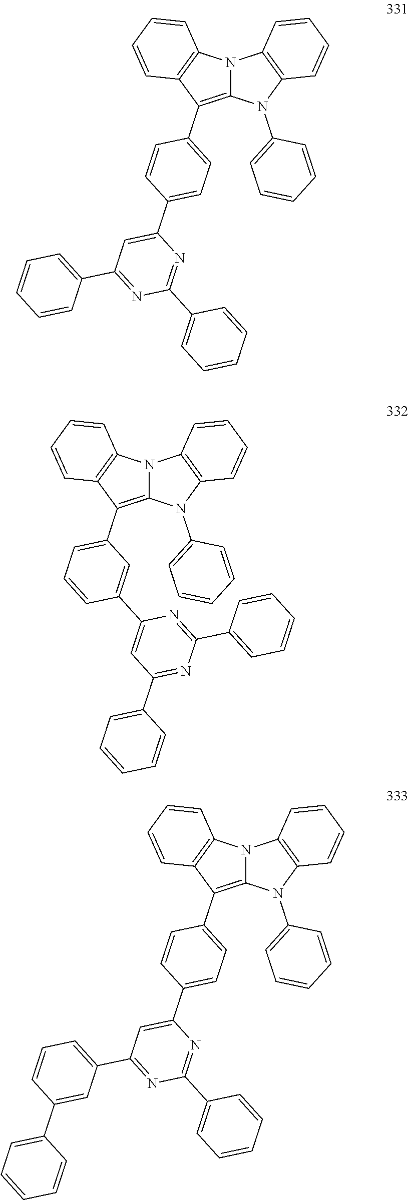

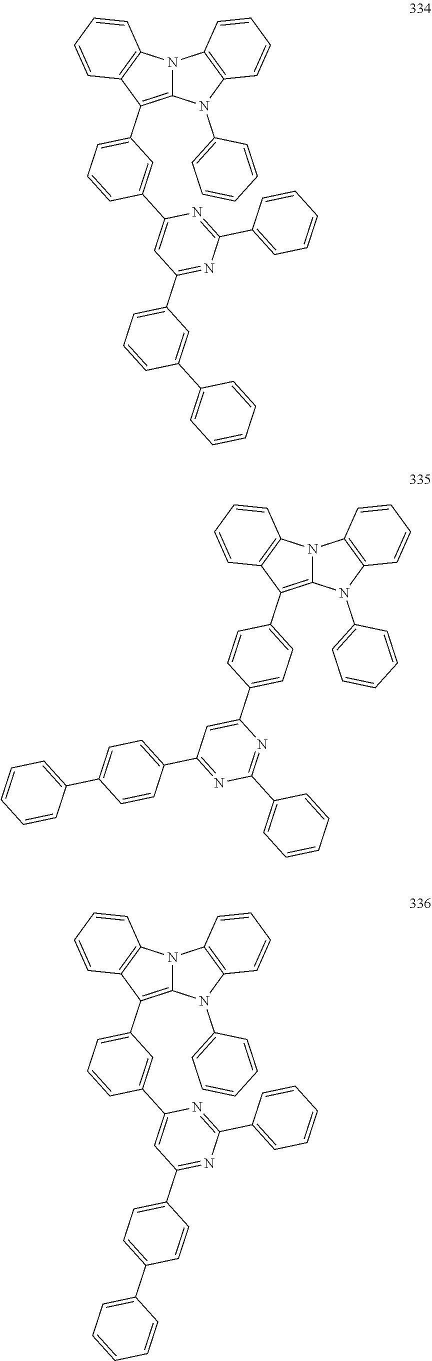 [SMILES: c1ccc(-c2cccc(-c3cc(-c4cccc(-c5c6ccccc6n6c7ccccc7n(-c7ccccc7)c56)c4)nc(-c4ccccc4)n3)c2)cc1 ; c1ccc(-c2ccc(-c3cc(-c4ccc(-c5c6ccccc6n6c7ccccc7n(-c7ccccc7)c56)cc4)nc(-c4ccccc4)n3)cc2)cc1 ; c1ccc(-c2ccc(-c3cc(-c4cccc(-c5c6ccccc6n6c7ccccc7n(-c7ccccc7)c56)c4)nc(-c4ccccc4)n3)cc2)cc1]

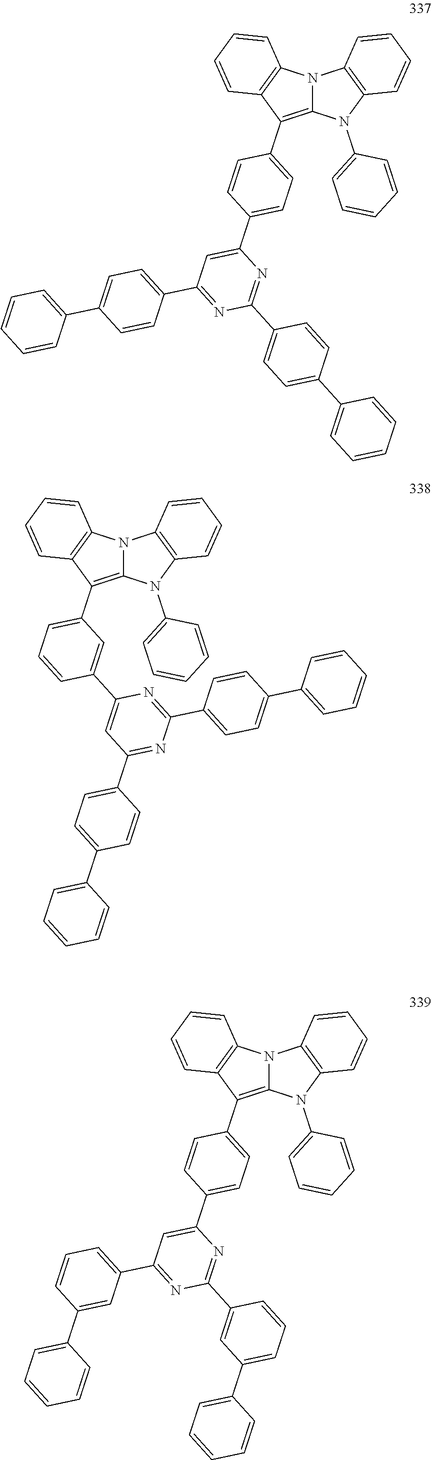 [SMILES: c1ccc(-c2ccc(-c3cc(-c4ccc(-c5c6ccccc6n6c7ccccc7n(-c7ccccc7)c56)cc4)nc(-c4ccc(-c5ccccc5)cc4)n3)cc2)cc1 ; c1ccc(-c2ccc(-c3cc(-c4cccc(-c5c6ccccc6n6c7ccccc7n(-c7ccccc7)c56)c4)nc(-c4ccc(-c5ccccc5)cc4)n3)cc2)cc1 ; c1ccc(-c2cccc(-c3cc(-c4ccc(-c5c6ccccc6n6c7ccccc7n(-c7ccccc7)c56)cc4)nc(-c4cccc(-c5ccccc5)c4)n3)c2)cc1]

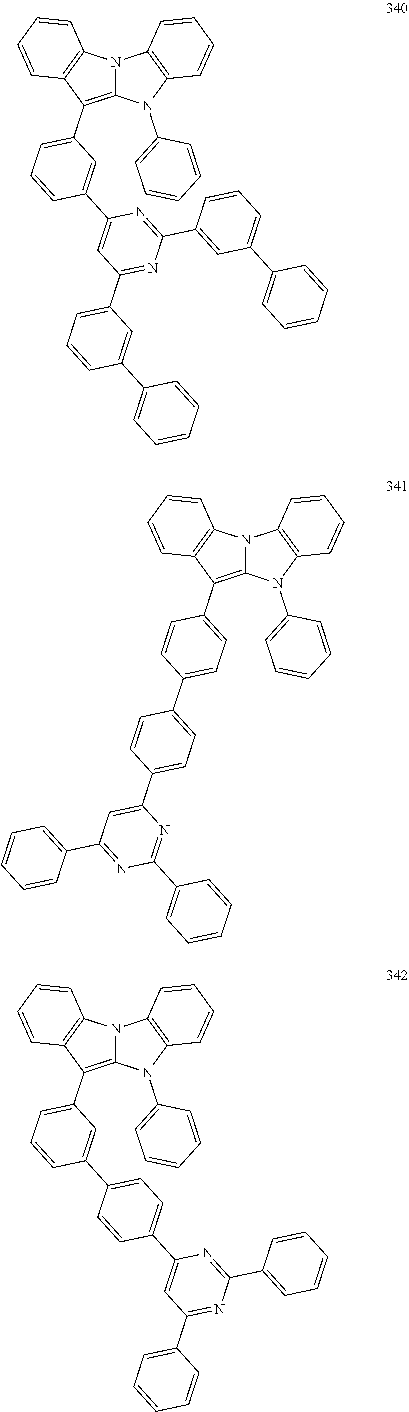

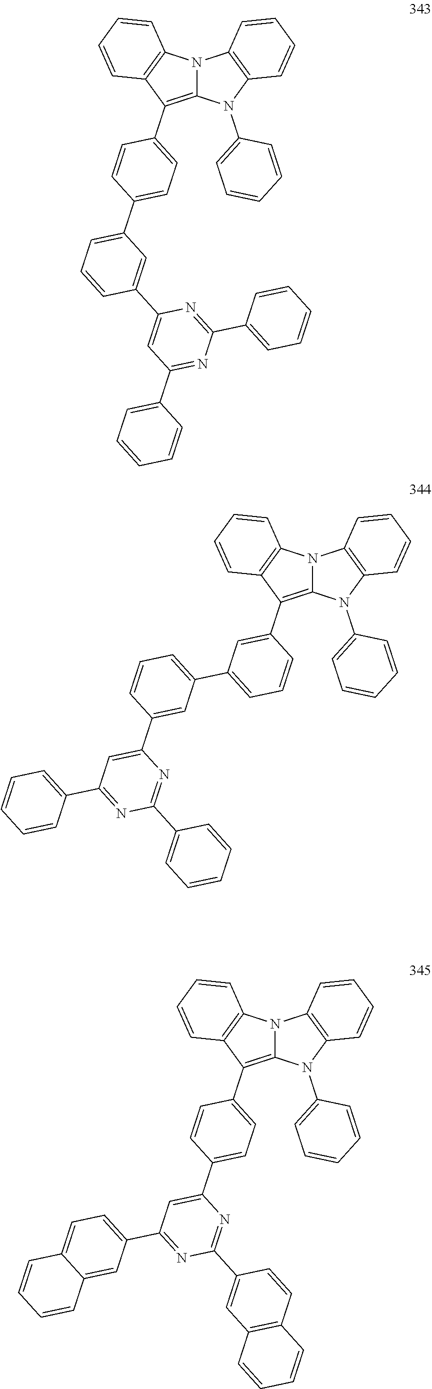 [SMILES: c1ccc(-c2cc(-c3cccc(-c4ccc(-c5c6ccccc6n6c7ccccc7n(-c7ccccc7)c56)cc4)c3)nc(-c3ccccc3)n2)cc1 ; c1ccc(-c2cc(-c3cccc(-c4cccc(-c5c6ccccc6n6c7ccccc7n(-c7ccccc7)c56)c4)c3)nc(-c3ccccc3)n2)cc1 ; c1ccc(-n2c3ccccc3n3c4ccccc4c(-c4ccc(-c5cc(-c6ccc7ccccc7c6)nc(-c6ccc7ccccc7c6)n5)cc4)c23)cc1]

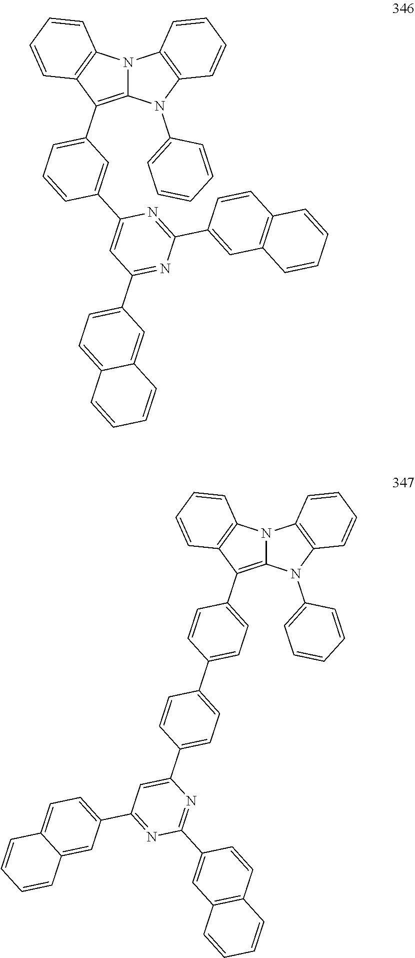 [SMILES: c1ccc(-n2c3ccccc3n3c4ccccc4c(-c4cccc(-c5cc(-c6ccc7ccccc7c6)nc(-c6ccc7ccccc7c6)n5)c4)c23)cc1 ; c1ccc(-n2c3ccccc3n3c4ccccc4c(-c4ccc(-c5ccc(-c6cc(-c7ccc8ccccc8c7)nc(-c7ccc8ccccc8c7)n6)cc5)cc4)c23)cc1]

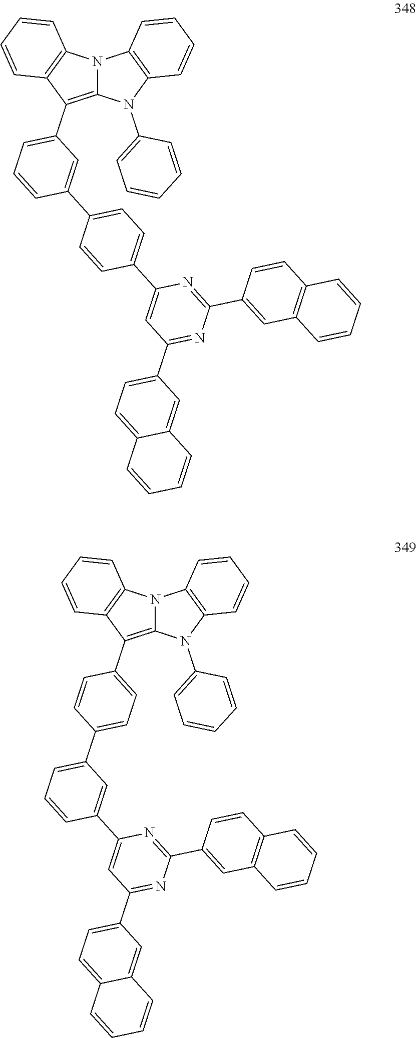 [SMILES: c1ccc(-n2c3ccccc3n3c4ccccc4c(-c4cccc(-c5ccc(-c6cc(-c7ccc8ccccc8c7)nc(-c7ccc8ccccc8c7)n6)cc5)c4)c23)cc1 ; c1ccc(-n2c3ccccc3n3c4ccccc4c(-c4ccc(-c5cccc(-c6cc(-c7ccc8ccccc8c7)nc(-c7ccc8ccccc8c7)n6)c5)cc4)c23)cc1]

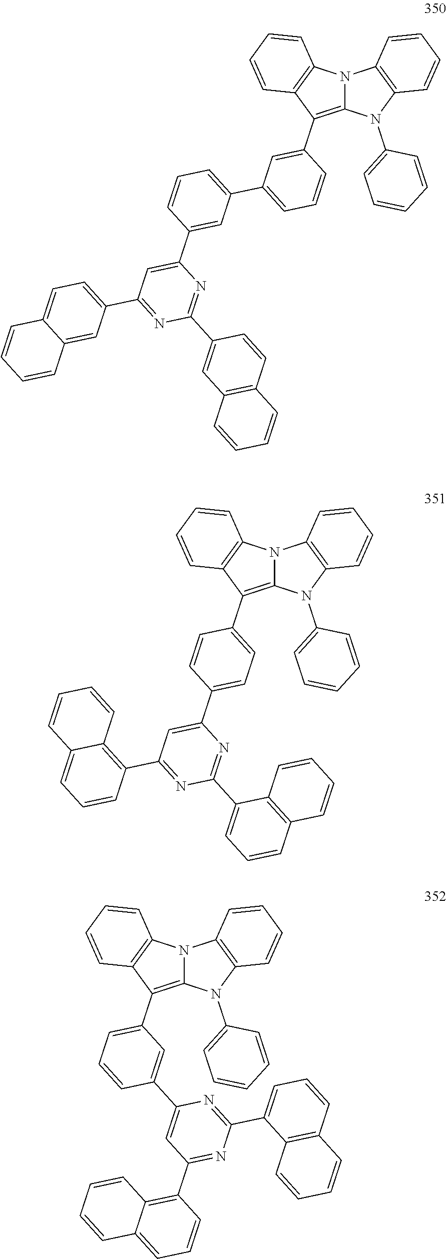 [SMILES: c1ccc(-n2c3ccccc3n3c4ccccc4c(-c4cccc(-c5cccc(-c6cc(-c7ccc8ccccc8c7)nc(-c7ccc8ccccc8c7)n6)c5)c4)c23)cc1 ; c1ccc(-n2c3ccccc3n3c4ccccc4c(-c4ccc(-c5cc(-c6cccc7ccccc67)nc(-c6cccc7ccccc67)n5)cc4)c23)cc1 ; c1ccc(-n2c3ccccc3n3c4ccccc4c(-c4cccc(-c5cc(-c6cccc7ccccc67)nc(-c6cccc7ccccc67)n5)c4)c23)cc1]

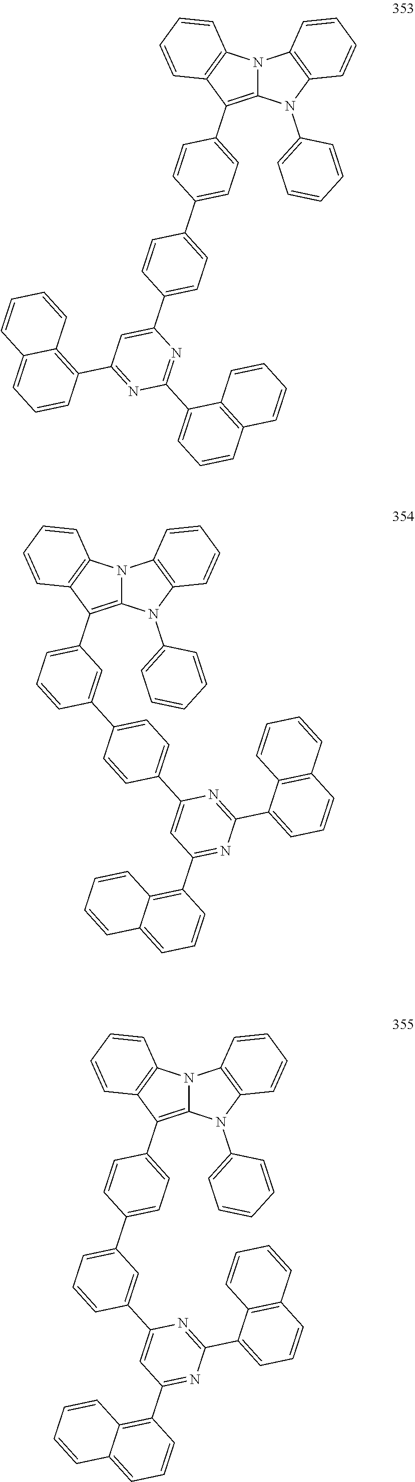 [SMILES: c1ccc(-n2c3ccccc3n3c4ccccc4c(-c4ccc(-c5ccc(-c6cc(-c7cccc8ccccc78)nc(-c7cccc8ccccc78)n6)cc5)cc4)c23)cc1 ; c1ccc(-n2c3ccccc3n3c4ccccc4c(-c4cccc(-c5ccc(-c6cc(-c7cccc8ccccc78)nc(-c7cccc8ccccc78)n6)cc5)c4)c23)cc1 ; c1ccc(-n2c3ccccc3n3c4ccccc4c(-c4ccc(-c5cccc(-c6cc(-c7cccc8ccccc78)nc(-c7cccc8ccccc78)n6)c5)cc4)c23)cc1]

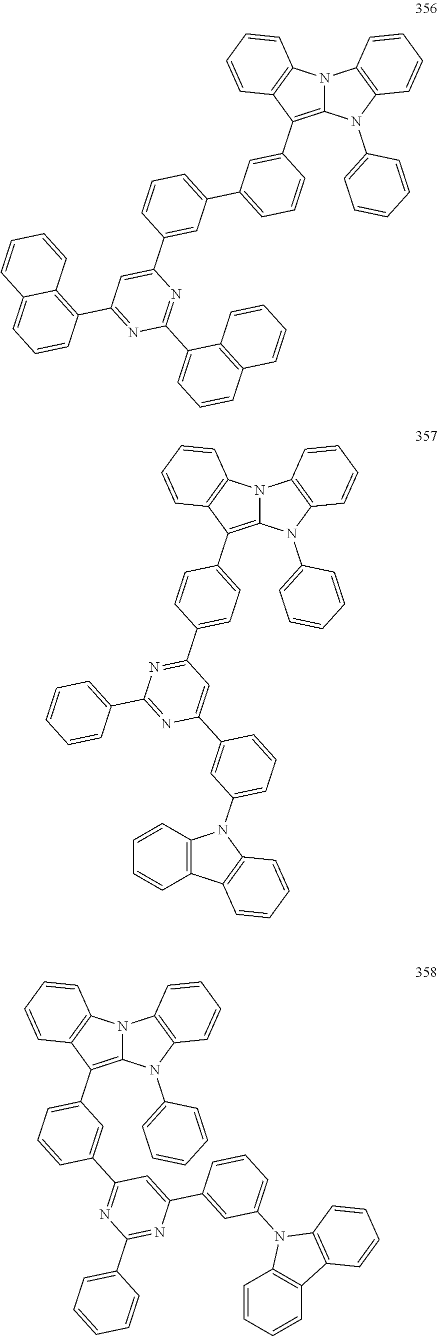

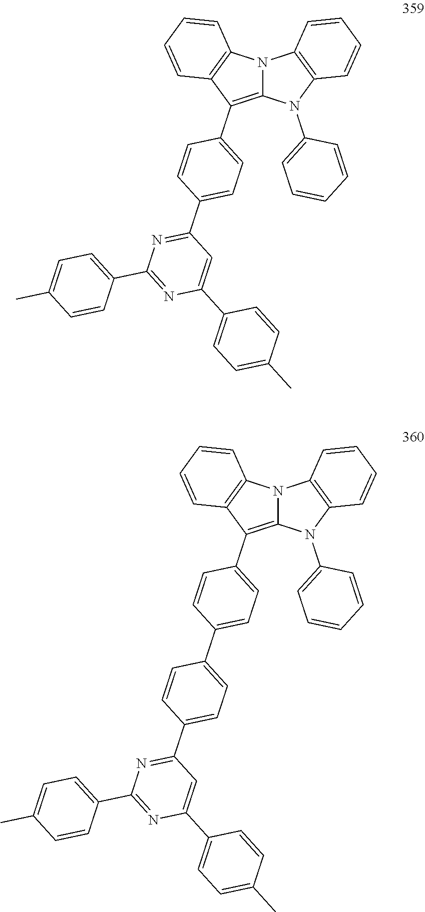 [SMILES: Cc1ccc(-c2cc(-c3ccc(-c4c5ccccc5n5c6ccccc6n(-c6ccccc6)c45)cc3)nc(-c3ccc(C)cc3)n2)cc1 ; Cc1ccc(-c2cc(-c3ccc(-c4ccc(-c5c6ccccc6n6c7ccccc7n(-c7ccccc7)c56)cc4)cc3)nc(-c3ccc(C)cc3)n2)cc1]

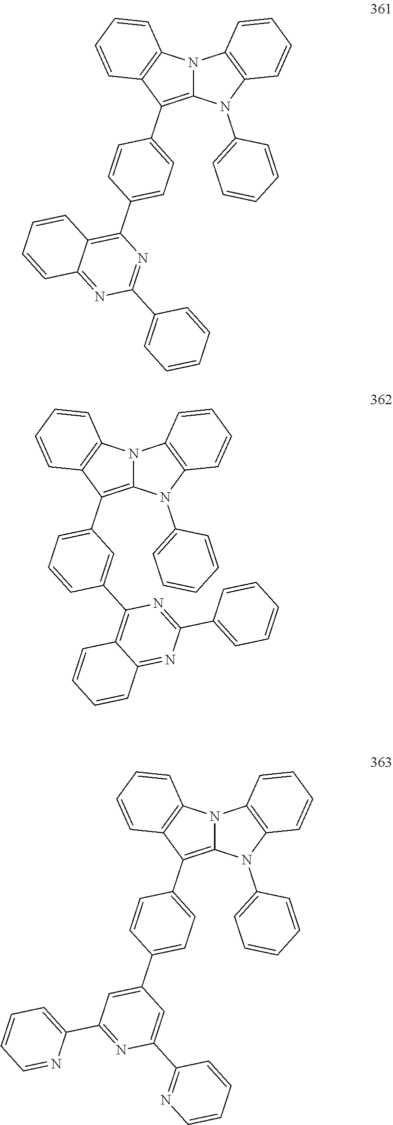

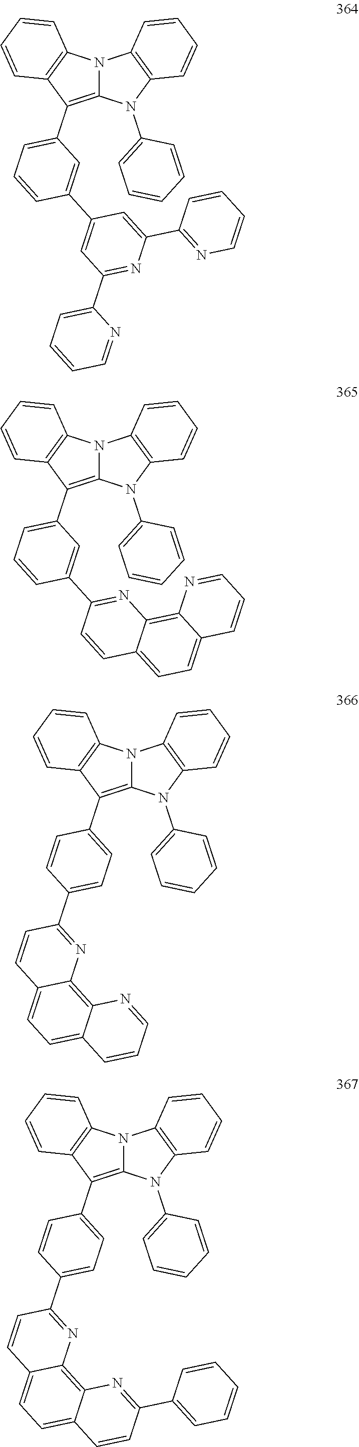

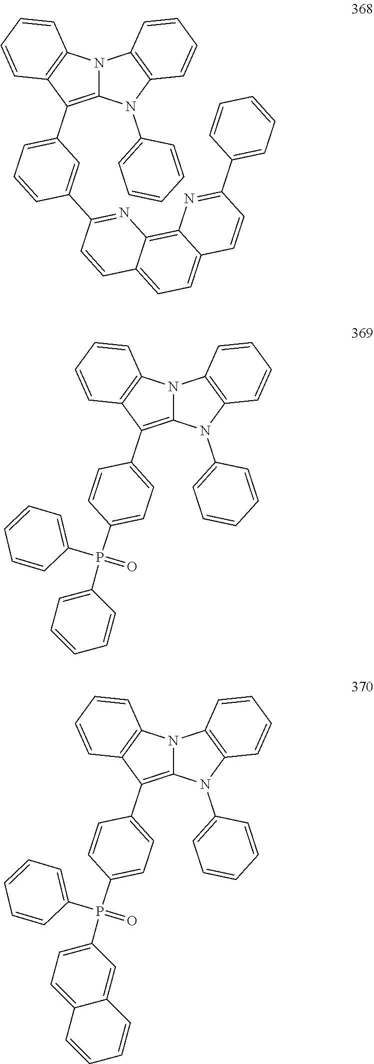 [SMILES: c1ccc(-c2ccc3ccc4ccc(-c5cccc(-c6c7ccccc7n7c8ccccc8n(-c8ccccc8)c67)c5)nc4c3n2)cc1 ; O=P(c1ccccc1)(c1ccccc1)c1ccc(-c2c3ccccc3n3c4ccccc4n(-c4ccccc4)c23)cc1 ; O=P(c1ccccc1)(c1ccc(-c2c3ccccc3n3c4ccccc4n(-c4ccccc4)c23)cc1)c1ccc2ccccc2c1]

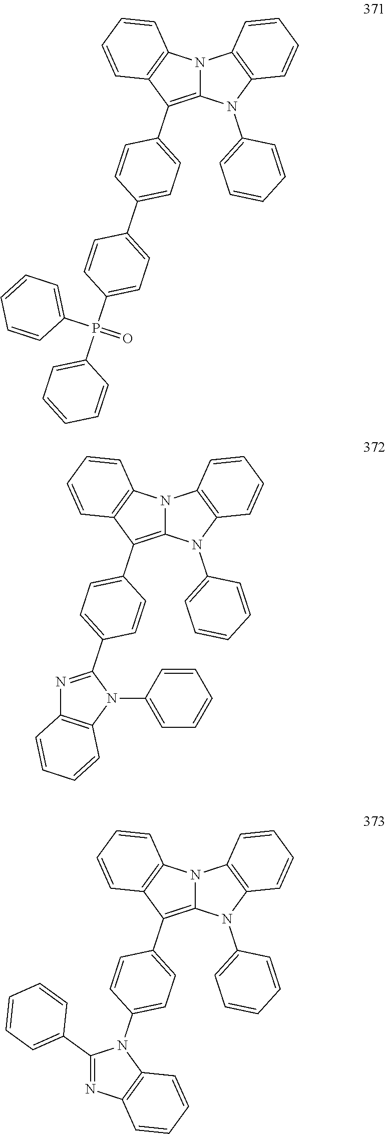

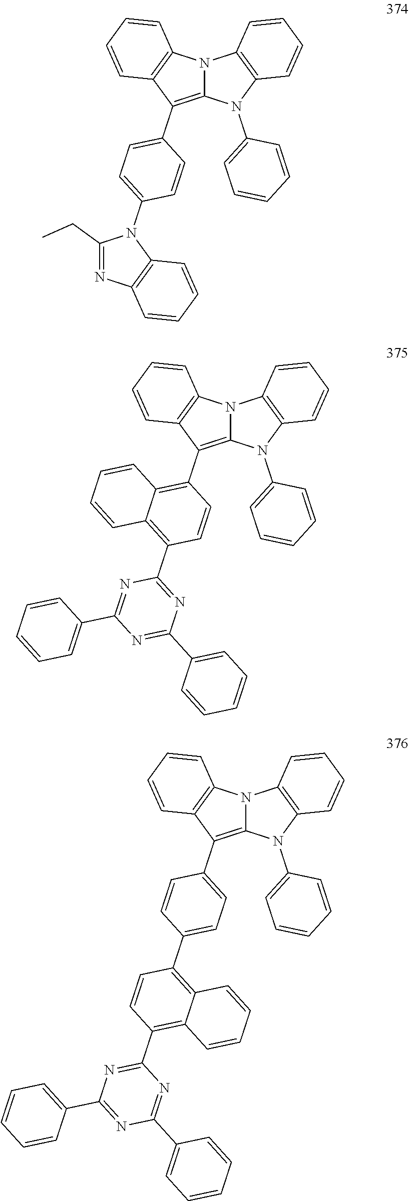

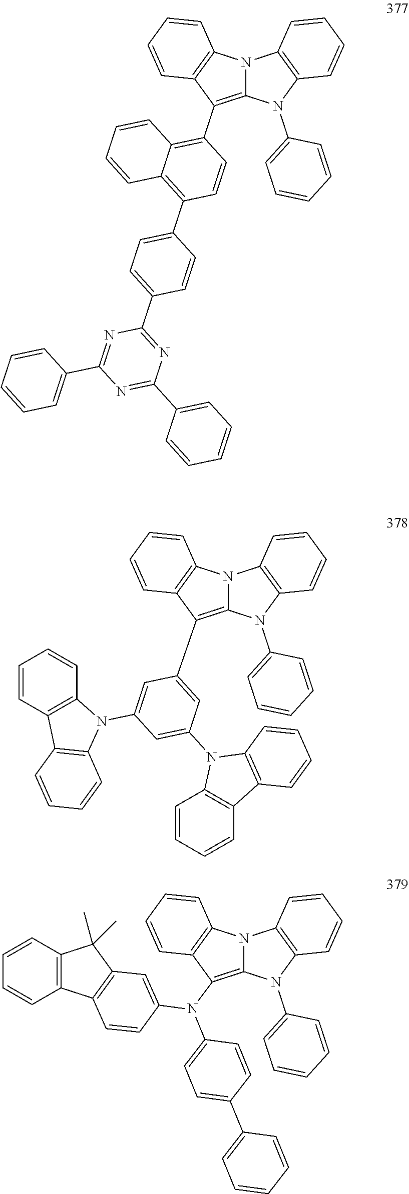

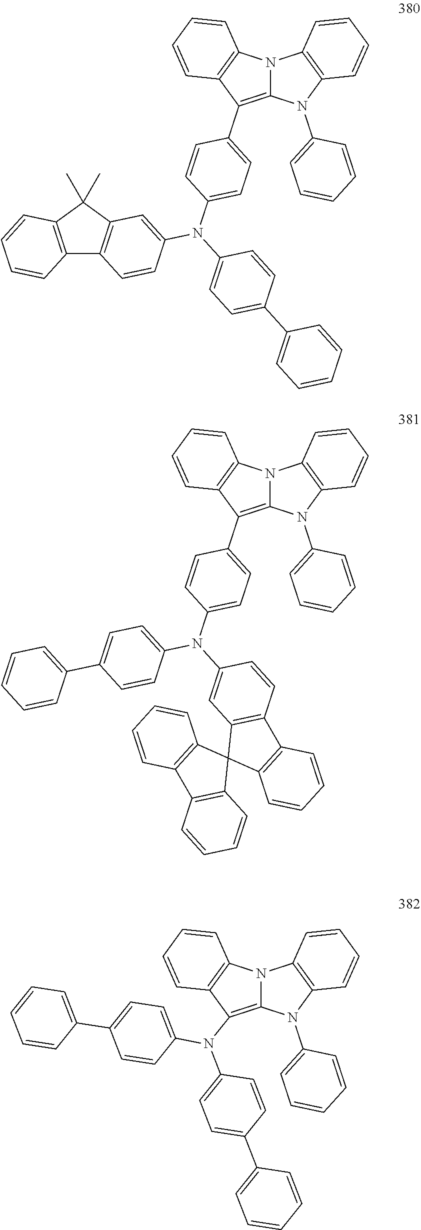 [SMILES: CC1(C)c2ccccc2-c2ccc(N(c3ccc(-c4ccccc4)cc3)c3ccc(-c4c5ccccc5n5c6ccccc6n(-c6ccccc6)c45)cc3)cc21 ; c1ccc(-c2ccc(N(c3ccc(-c4c5ccccc5n5c6ccccc6n(-c6ccccc6)c45)cc3)c3ccc4c(c3)C3(c5ccccc5-c5ccccc53)c3ccccc3-4)cc2)cc1 ; c1ccc(-c2ccc(N(c3ccc(-c4ccccc4)cc3)c3c4ccccc4n4c5ccccc5n(-c5ccccc5)c34)cc2)cc1]

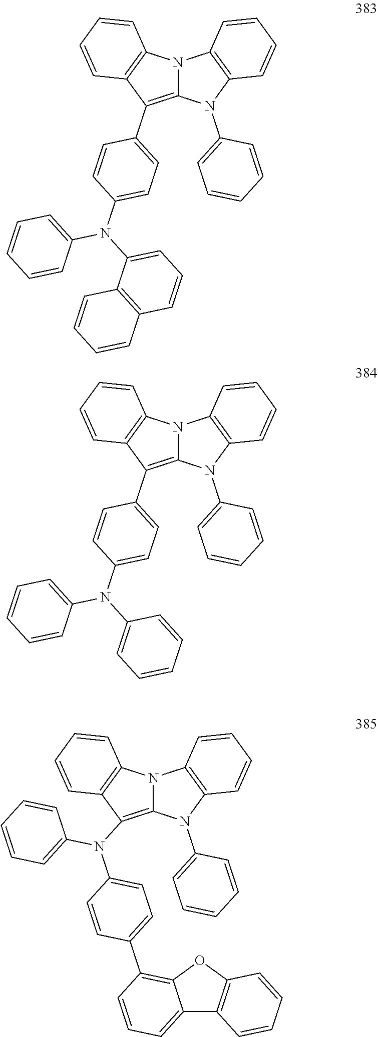

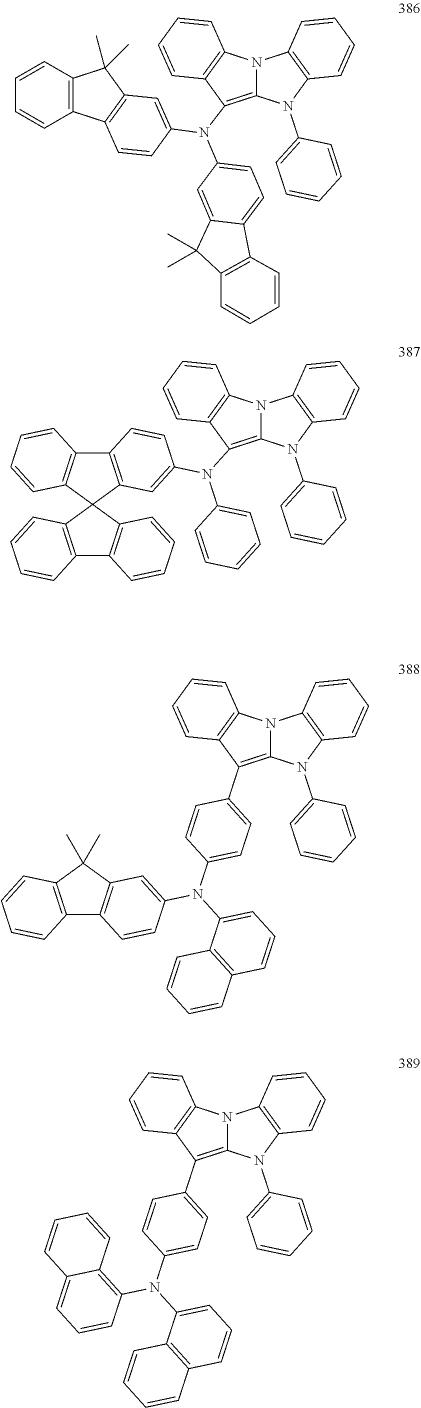 [SMILES: CC1(C)c2ccccc2-c2ccc(N(c3ccc4c(c3)C(C)(C)c3ccccc3-4)c3c4ccccc4n4c5ccccc5n(-c5ccccc5)c34)cc21 ; c1ccc(N(c2ccc3c(c2)C2(c4ccccc4-c4ccccc42)c2ccccc2-3)c2c3ccccc3n3c4ccccc4n(-c4ccccc4)c23)cc1 ; CC1(C)c2ccccc2-c2ccc(N(c3ccc(-c4c5ccccc5n5c6ccccc6n(-c6ccccc6)c45)cc3)c3cccc4ccccc34)cc21 ; c1ccc(-n2c3ccccc3n3c4ccccc4c(-c4ccc(N(c5cccc6ccccc56)c5cccc6ccccc56)cc4)c23)cc1]

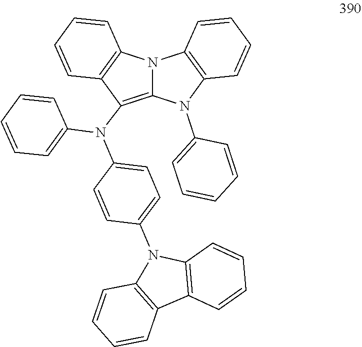 [SMILES: c1ccc(N(c2ccc(-n3c4ccccc4c4ccccc43)cc2)c2c3ccccc3n3c4ccccc4n(-c4ccccc4)c23)cc1]

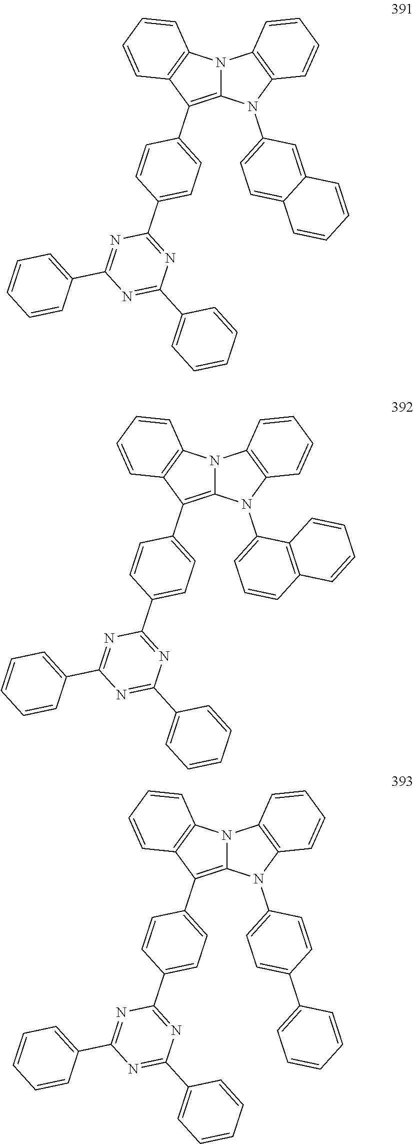

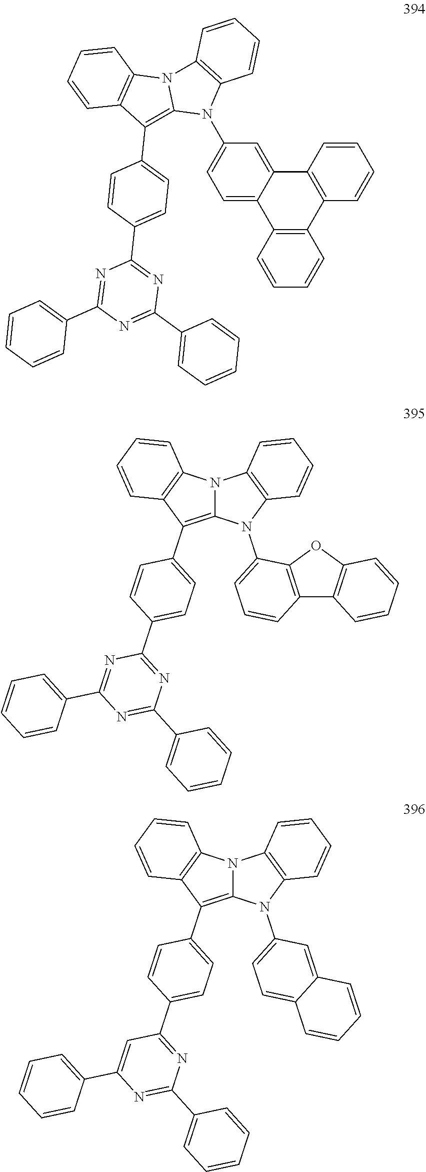

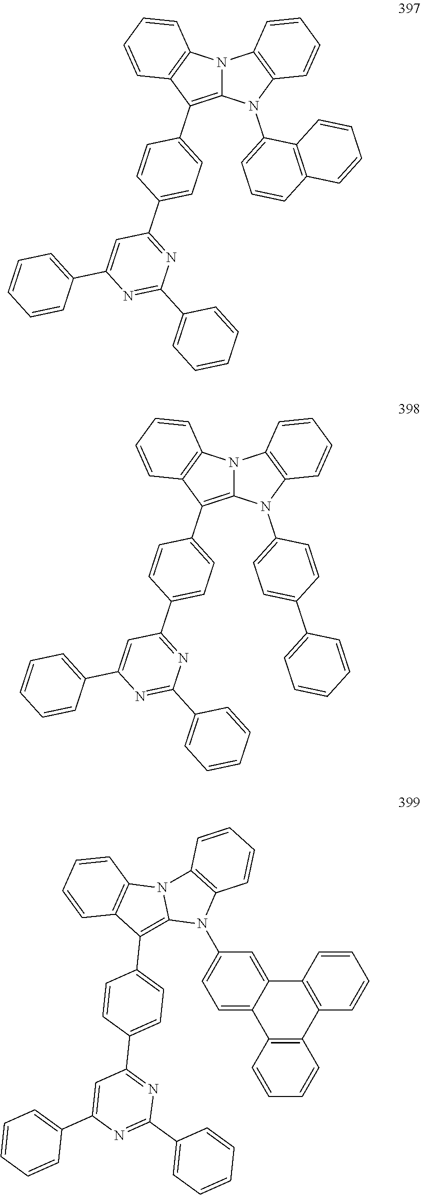 [SMILES: c1ccc(-c2cc(-c3ccc(-c4c5ccccc5n5c6ccccc6n(-c6cccc7ccccc67)c45)cc3)nc(-c3ccccc3)n2)cc1 ; c1ccc(-c2ccc(-n3c4ccccc4n4c5ccccc5c(-c5ccc(-c6cc(-c7ccccc7)nc(-c7ccccc7)n6)cc5)c34)cc2)cc1 ; c1ccc(-c2cc(-c3ccc(-c4c5ccccc5n5c6ccccc6n(-c6ccc7c8ccccc8c8ccccc8c7c6)c45)cc3)nc(-c3ccccc3)n2)cc1]

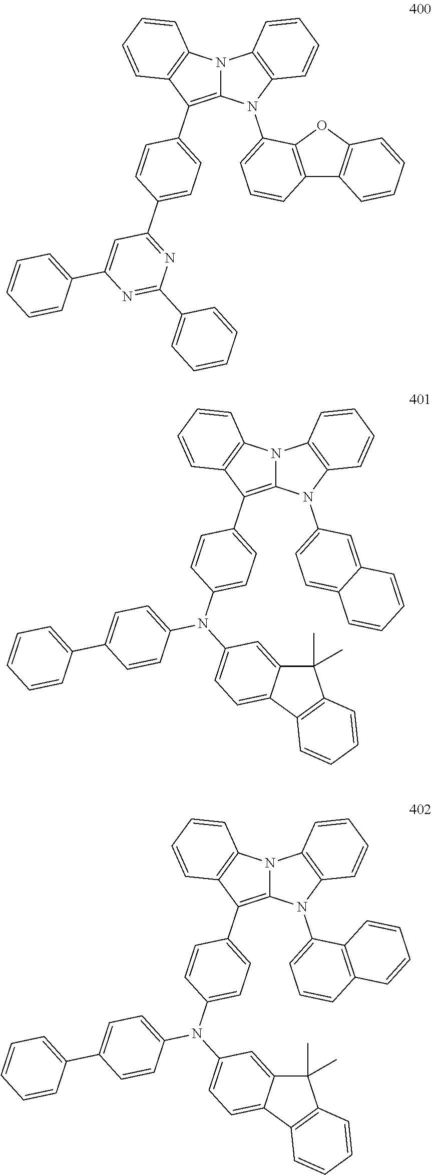

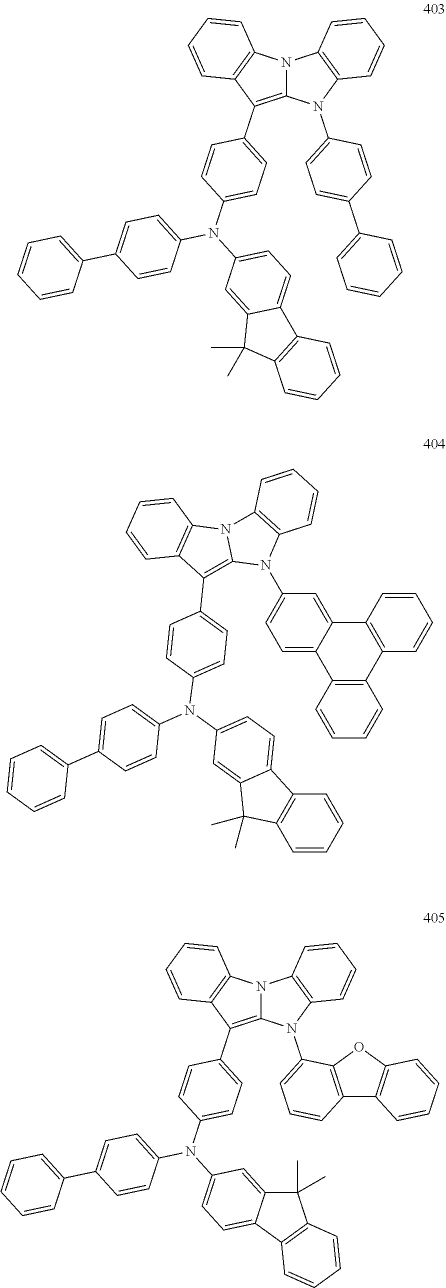 [SMILES: CC1(C)c2ccccc2-c2ccc(N(c3ccc(-c4ccccc4)cc3)c3ccc(-c4c5ccccc5n5c6ccccc6n(-c6ccc(-c7ccccc7)cc6)c45)cc3)cc21 ; CC1(C)c2ccccc2-c2ccc(N(c3ccc(-c4ccccc4)cc3)c3ccc(-c4c5ccccc5n5c6ccccc6n(-c6ccc7c8ccccc8c8ccccc8c7c6)c45)cc3)cc21 ; CC1(C)c2ccccc2-c2ccc(N(c3ccc(-c4ccccc4)cc3)c3ccc(-c4c5ccccc5n5c6ccccc6n(-c6cccc7c6oc6ccccc67)c45)cc3)cc21]

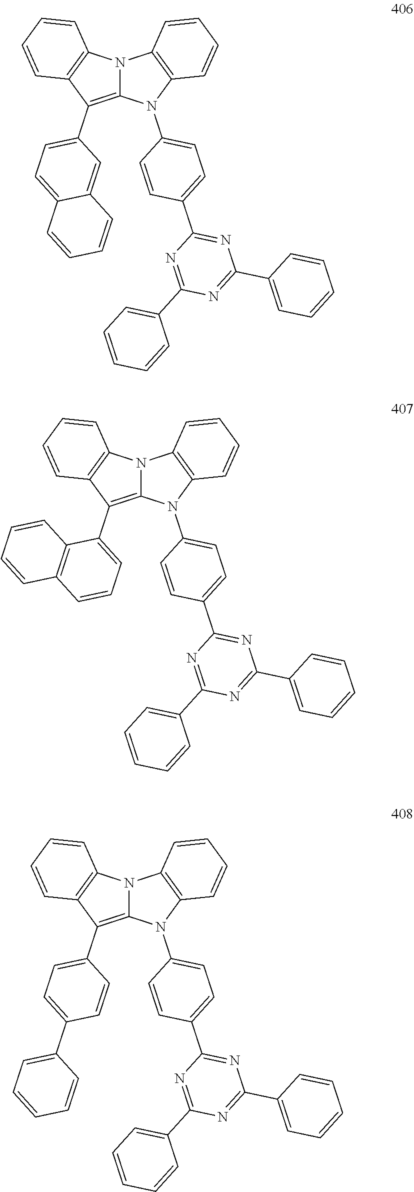 [SMILES: c1ccc(-c2nc(-c3ccccc3)nc(-c3ccc(-n4c5ccccc5n5c6ccccc6c(-c6ccc7ccccc7c6)c45)cc3)n2)cc1 ; c1ccc(-c2nc(-c3ccccc3)nc(-c3ccc(-n4c5ccccc5n5c6ccccc6c(-c6cccc7ccccc67)c45)cc3)n2)cc1 ; c1ccc(-c2ccc(-c3c4ccccc4n4c5ccccc5n(-c5ccc(-c6nc(-c7ccccc7)nc(-c7ccccc7)n6)cc5)c34)cc2)cc1]

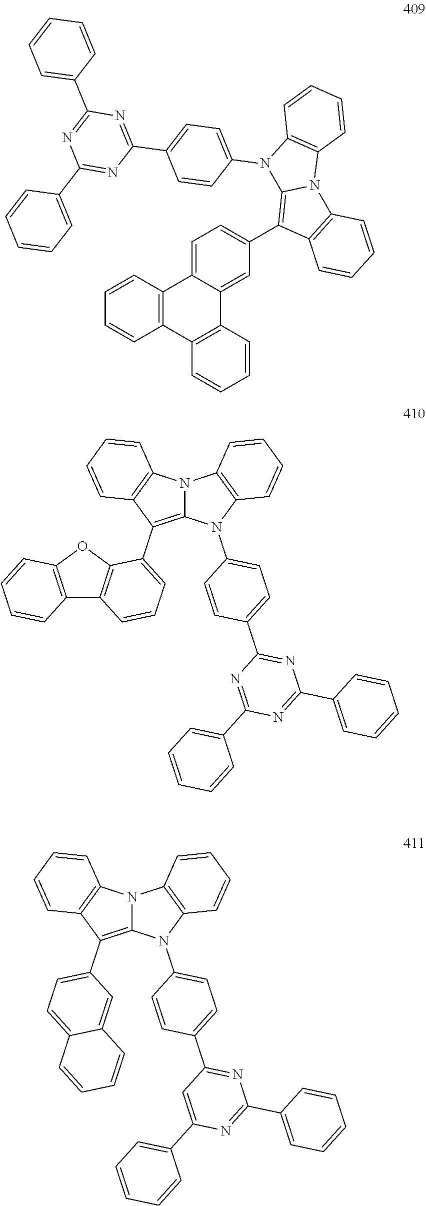 [SMILES: c1ccc(-c2nc(-c3ccccc3)nc(-c3ccc(-n4c5ccccc5n5c6ccccc6c(-c6ccc7c8ccccc8c8ccccc8c7c6)c45)cc3)n2)cc1 ; c1ccc(-c2nc(-c3ccccc3)nc(-c3ccc(-n4c5ccccc5n5c6ccccc6c(-c6cccc7c6oc6ccccc67)c45)cc3)n2)cc1 ; c1ccc(-c2cc(-c3ccc(-n4c5ccccc5n5c6ccccc6c(-c6ccc7ccccc7c6)c45)cc3)nc(-c3ccccc3)n2)cc1]

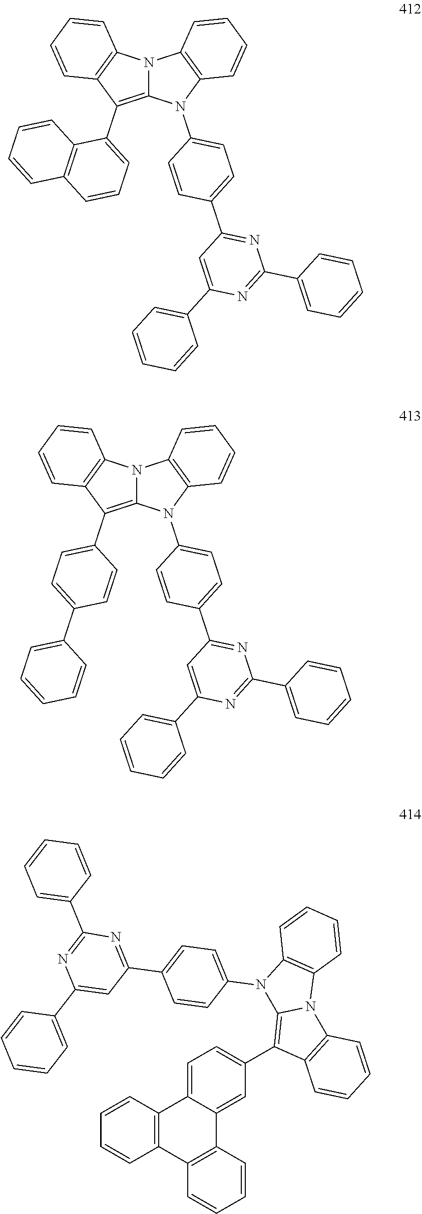 [SMILES: c1ccc(-c2cc(-c3ccc(-n4c5ccccc5n5c6ccccc6c(-c6cccc7ccccc67)c45)cc3)nc(-c3ccccc3)n2)cc1 ; c1ccc(-c2ccc(-c3c4ccccc4n4c5ccccc5n(-c5ccc(-c6cc(-c7ccccc7)nc(-c7ccccc7)n6)cc5)c34)cc2)cc1 ; c1ccc(-c2cc(-c3ccc(-n4c5ccccc5n5c6ccccc6c(-c6ccc7c8ccccc8c8ccccc8c7c6)c45)cc3)nc(-c3ccccc3)n2)cc1]

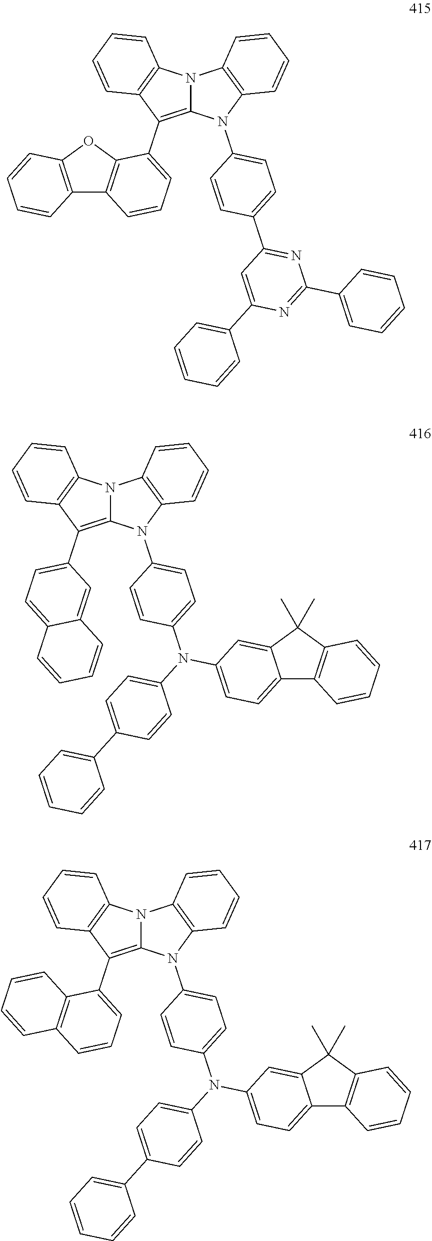 [SMILES: c1ccc(-c2cc(-c3ccc(-n4c5ccccc5n5c6ccccc6c(-c6cccc7c6oc6ccccc67)c45)cc3)nc(-c3ccccc3)n2)cc1 ; CC1(C)c2ccccc2-c2ccc(N(c3ccc(-c4ccccc4)cc3)c3ccc(-n4c5ccccc5n5c6ccccc6c(-c6ccc7ccccc7c6)c45)cc3)cc21 ; CC1(C)c2ccccc2-c2ccc(N(c3ccc(-c4ccccc4)cc3)c3ccc(-n4c5ccccc5n5c6ccccc6c(-c6cccc7ccccc67)c45)cc3)cc21]

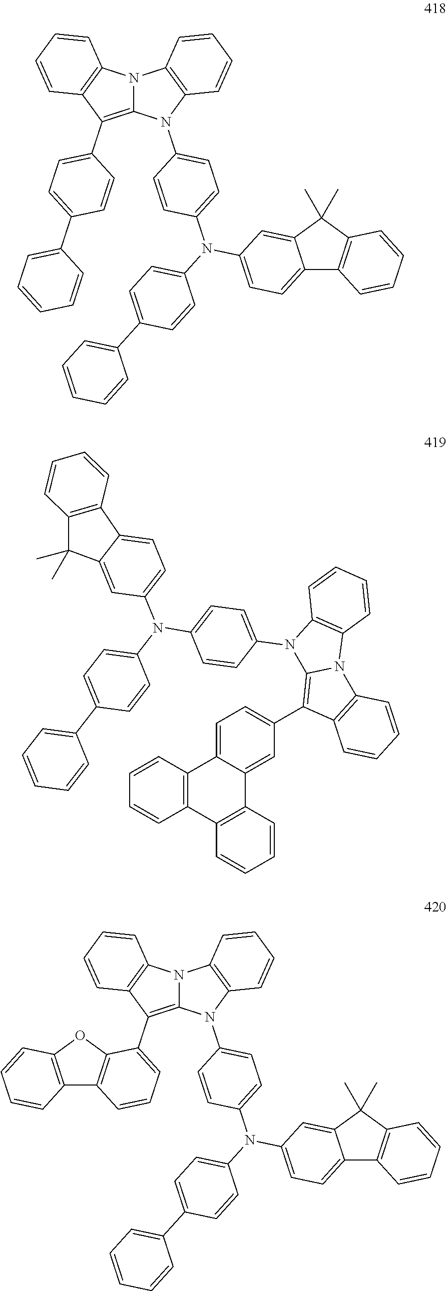 [SMILES: CC1(C)c2ccccc2-c2ccc(N(c3ccc(-c4ccccc4)cc3)c3ccc(-n4c5ccccc5n5c6ccccc6c(-c6ccc(-c7ccccc7)cc6)c45)cc3)cc21 ; CC1(C)c2ccccc2-c2ccc(N(c3ccc(-c4ccccc4)cc3)c3ccc(-n4c5ccccc5n5c6ccccc6c(-c6ccc7c8ccccc8c8ccccc8c7c6)c45)cc3)cc21 ; CC1(C)c2ccccc2-c2ccc(N(c3ccc(-c4ccccc4)cc3)c3ccc(-n4c5ccccc5n5c6ccccc6c(-c6cccc7c6oc6ccccc67)c45)cc3)cc21]

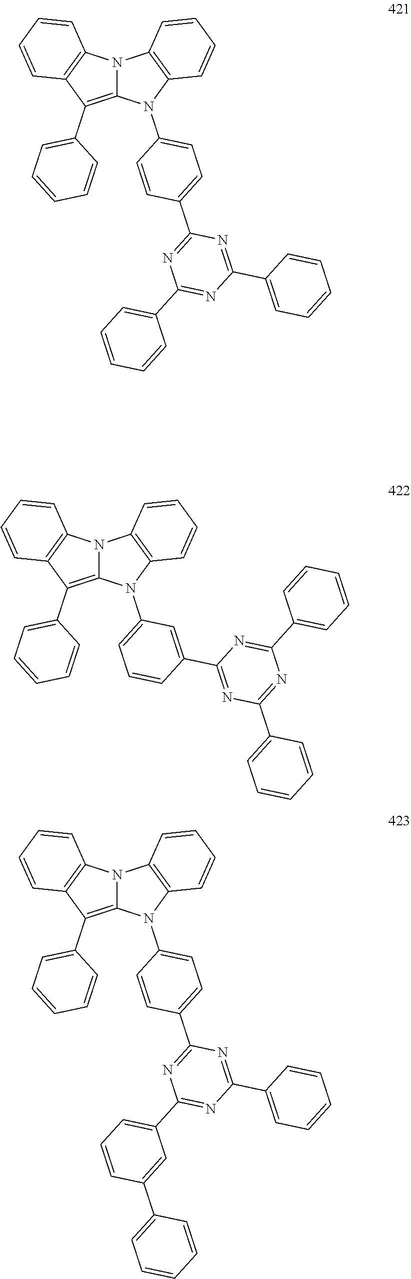 [SMILES: c1ccc(-c2nc(-c3ccccc3)nc(-c3ccc(-n4c5ccccc5n5c6ccccc6c(-c6ccccc6)c45)cc3)n2)cc1 ; c1ccc(-c2nc(-c3ccccc3)nc(-c3cccc(-n4c5ccccc5n5c6ccccc6c(-c6ccccc6)c45)c3)n2)cc1 ; c1ccc(-c2cccc(-c3nc(-c4ccccc4)nc(-c4ccc(-n5c6ccccc6n6c7ccccc7c(-c7ccccc7)c56)cc4)n3)c2)cc1]

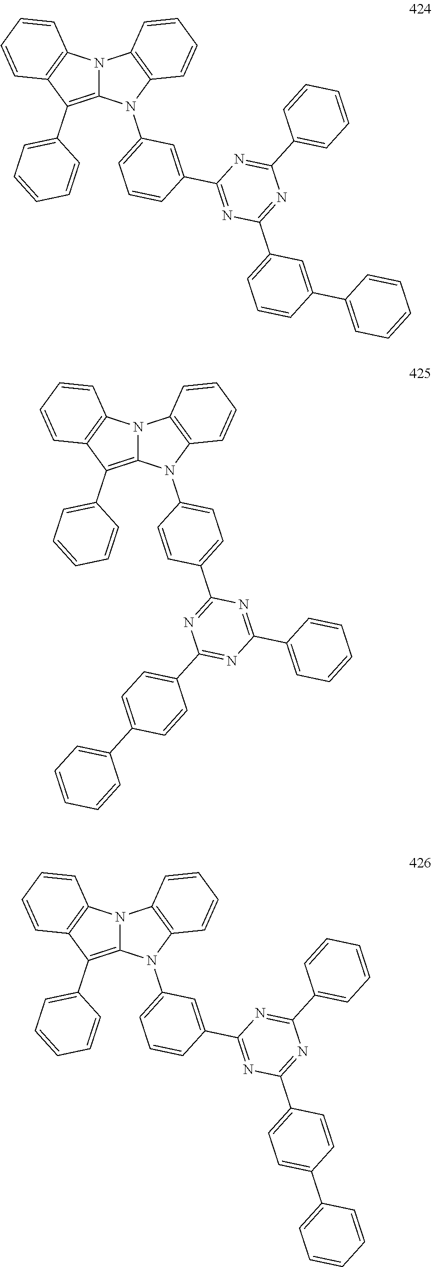 [SMILES: c1ccc(-c2cccc(-c3nc(-c4ccccc4)nc(-c4cccc(-n5c6ccccc6n6c7ccccc7c(-c7ccccc7)c56)c4)n3)c2)cc1 ; c1ccc(-c2ccc(-c3nc(-c4ccccc4)nc(-c4ccc(-n5c6ccccc6n6c7ccccc7c(-c7ccccc7)c56)cc4)n3)cc2)cc1 ; c1ccc(-c2ccc(-c3nc(-c4ccccc4)nc(-c4cccc(-n5c6ccccc6n6c7ccccc7c(-c7ccccc7)c56)c4)n3)cc2)cc1]

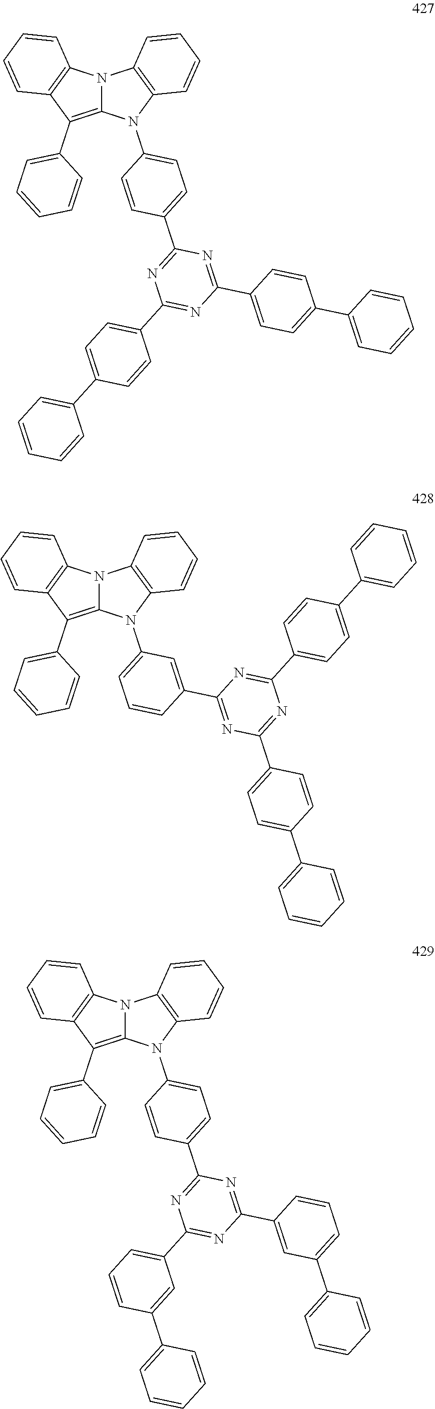 [SMILES: c1ccc(-c2ccc(-c3nc(-c4ccc(-c5ccccc5)cc4)nc(-c4ccc(-n5c6ccccc6n6c7ccccc7c(-c7ccccc7)c56)cc4)n3)cc2)cc1 ; c1ccc(-c2ccc(-c3nc(-c4ccc(-c5ccccc5)cc4)nc(-c4cccc(-n5c6ccccc6n6c7ccccc7c(-c7ccccc7)c56)c4)n3)cc2)cc1 ; c1ccc(-c2cccc(-c3nc(-c4ccc(-n5c6ccccc6n6c7ccccc7c(-c7ccccc7)c56)cc4)nc(-c4cccc(-c5ccccc5)c4)n3)c2)cc1]

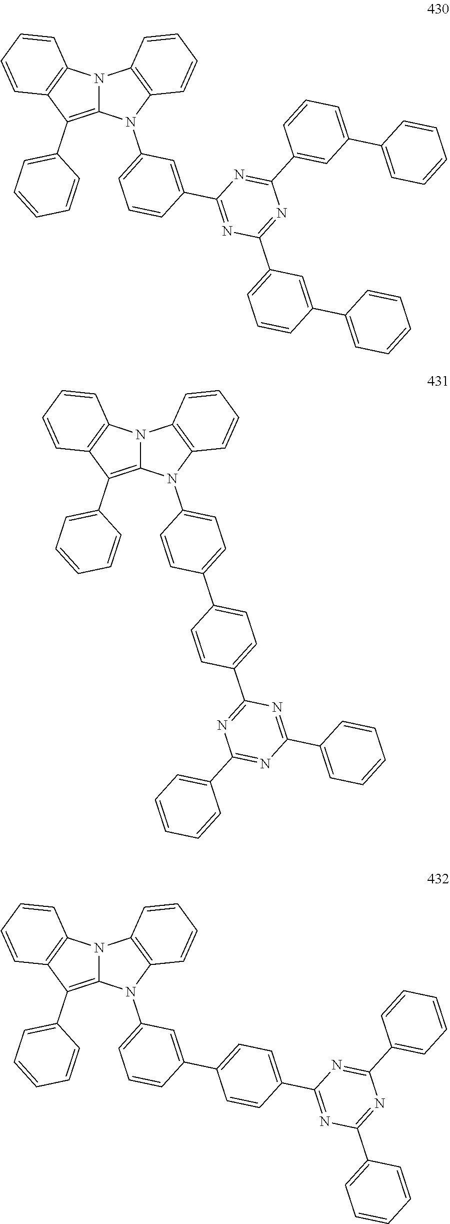 [SMILES: c1ccc(-c2cccc(-c3nc(-c4cccc(-c5ccccc5)c4)nc(-c4cccc(-n5c6ccccc6n6c7ccccc7c(-c7ccccc7)c56)c4)n3)c2)cc1 ; c1ccc(-c2nc(-c3ccccc3)nc(-c3ccc(-c4ccc(-n5c6ccccc6n6c7ccccc7c(-c7ccccc7)c56)cc4)cc3)n2)cc1 ; c1ccc(-c2nc(-c3ccccc3)nc(-c3ccc(-c4cccc(-n5c6ccccc6n6c7ccccc7c(-c7ccccc7)c56)c4)cc3)n2)cc1]

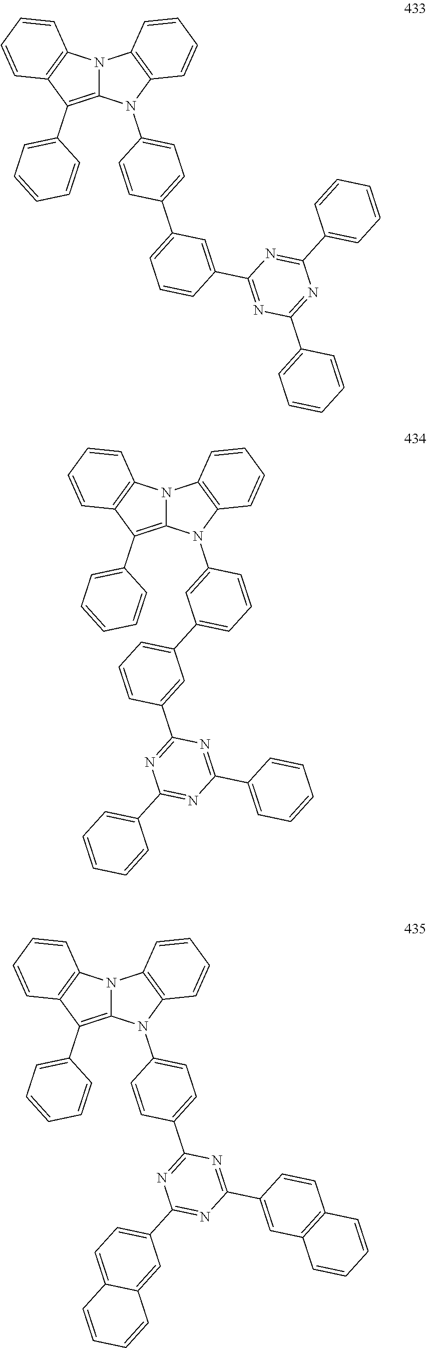 [SMILES: c1ccc(-c2nc(-c3ccccc3)nc(-c3cccc(-c4ccc(-n5c6ccccc6n6c7ccccc7c(-c7ccccc7)c56)cc4)c3)n2)cc1 ; c1ccc(-c2nc(-c3ccccc3)nc(-c3cccc(-c4cccc(-n5c6ccccc6n6c7ccccc7c(-c7ccccc7)c56)c4)c3)n2)cc1 ; c1ccc(-c2c3ccccc3n3c4ccccc4n(-c4ccc(-c5nc(-c6ccc7ccccc7c6)nc(-c6ccc7ccccc7c6)n5)cc4)c23)cc1]

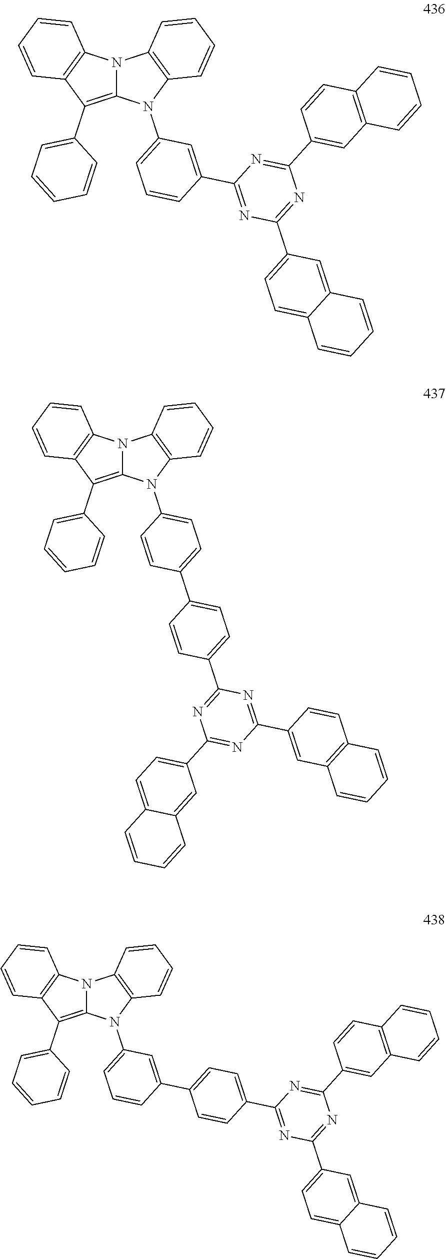 [SMILES: c1ccc(-c2c3ccccc3n3c4ccccc4n(-c4cccc(-c5nc(-c6ccc7ccccc7c6)nc(-c6ccc7ccccc7c6)n5)c4)c23)cc1 ; c1ccc(-c2c3ccccc3n3c4ccccc4n(-c4ccc(-c5ccc(-c6nc(-c7ccc8ccccc8c7)nc(-c7ccc8ccccc8c7)n6)cc5)cc4)c23)cc1 ; c1ccc(-c2c3ccccc3n3c4ccccc4n(-c4cccc(-c5ccc(-c6nc(-c7ccc8ccccc8c7)nc(-c7ccc8ccccc8c7)n6)cc5)c4)c23)cc1]

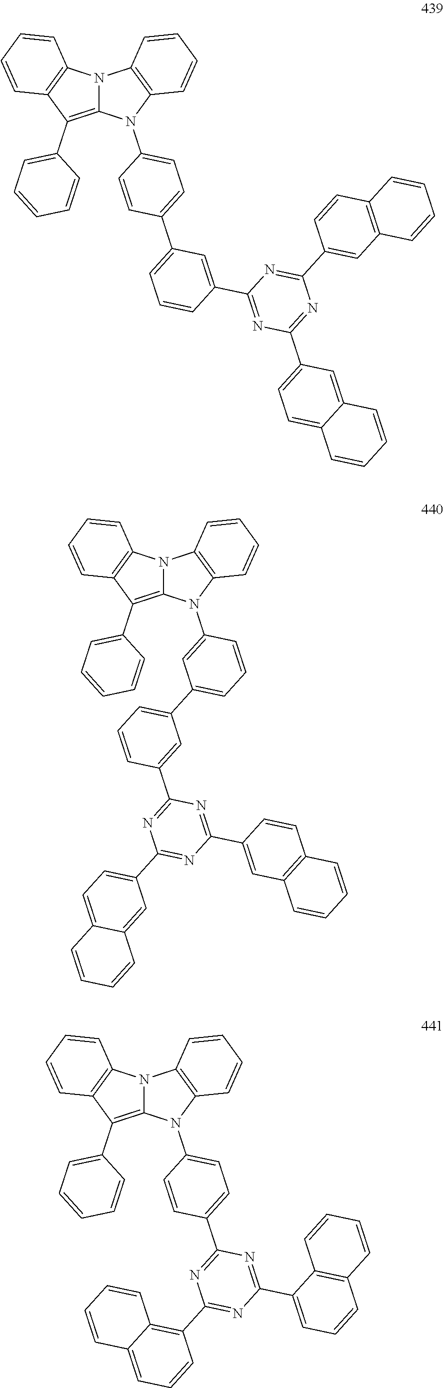 [SMILES: c1ccc(-c2c3ccccc3n3c4ccccc4n(-c4ccc(-c5cccc(-c6nc(-c7ccc8ccccc8c7)nc(-c7ccc8ccccc8c7)n6)c5)cc4)c23)cc1 ; c1ccc(-c2c3ccccc3n3c4ccccc4n(-c4cccc(-c5cccc(-c6nc(-c7ccc8ccccc8c7)nc(-c7ccc8ccccc8c7)n6)c5)c4)c23)cc1 ; c1ccc(-c2c3ccccc3n3c4ccccc4n(-c4ccc(-c5nc(-c6cccc7ccccc67)nc(-c6cccc7ccccc67)n5)cc4)c23)cc1]

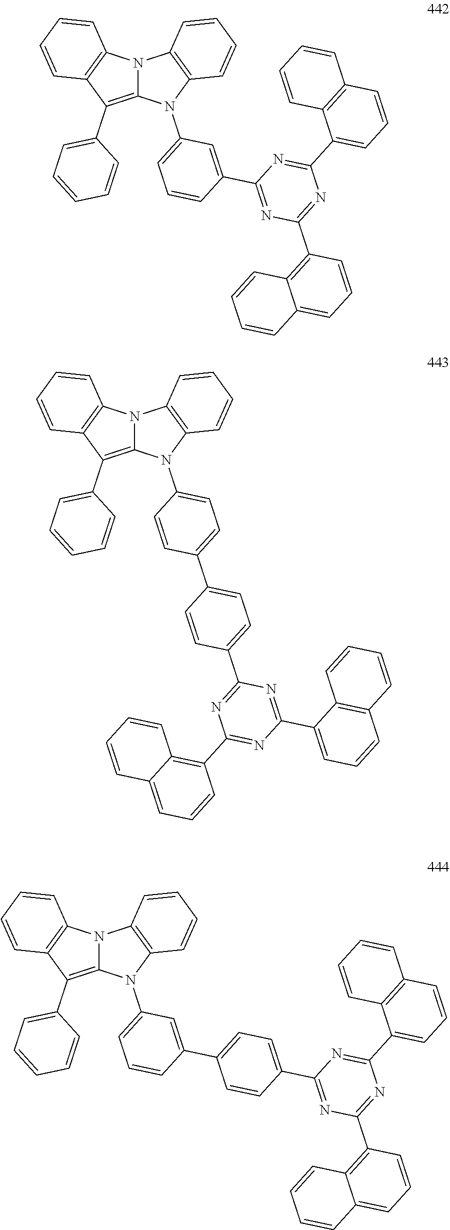 [SMILES: c1ccc(-c2c3ccccc3n3c4ccccc4n(-c4cccc(-c5nc(-c6cccc7ccccc67)nc(-c6cccc7ccccc67)n5)c4)c23)cc1 ; c1ccc(-c2c3ccccc3n3c4ccccc4n(-c4ccc(-c5ccc(-c6nc(-c7cccc8ccccc78)nc(-c7cccc8ccccc78)n6)cc5)cc4)c23)cc1 ; c1ccc(-c2c3ccccc3n3c4ccccc4n(-c4cccc(-c5ccc(-c6nc(-c7cccc8ccccc78)nc(-c7cccc8ccccc78)n6)cc5)c4)c23)cc1]

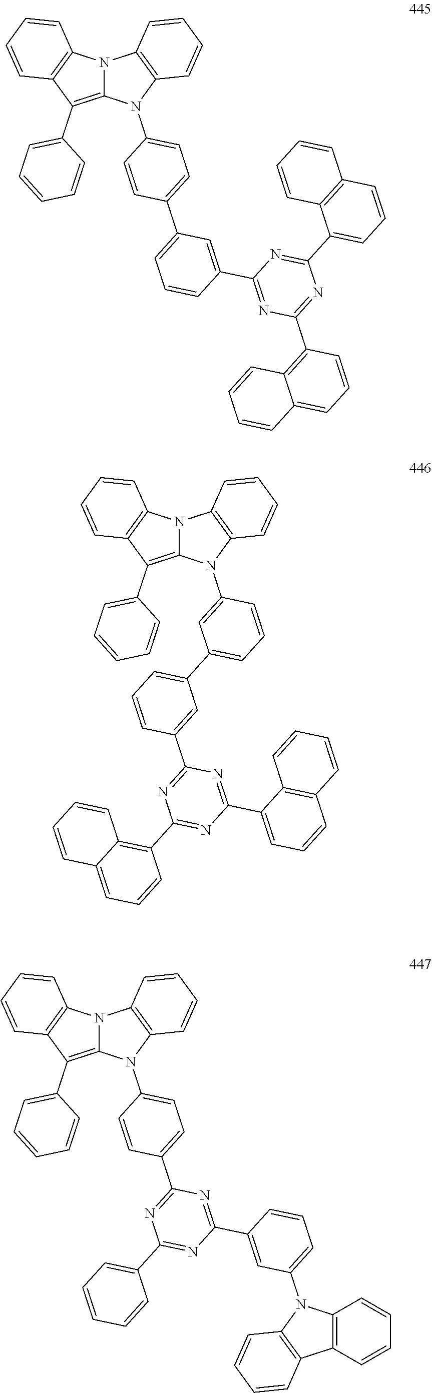 [SMILES: c1ccc(-c2c3ccccc3n3c4ccccc4n(-c4ccc(-c5cccc(-c6nc(-c7cccc8ccccc78)nc(-c7cccc8ccccc78)n6)c5)cc4)c23)cc1 ; c1ccc(-c2c3ccccc3n3c4ccccc4n(-c4cccc(-c5cccc(-c6nc(-c7cccc8ccccc78)nc(-c7cccc8ccccc78)n6)c5)c4)c23)cc1 ; c1ccc(-c2nc(-c3ccc(-n4c5ccccc5n5c6ccccc6c(-c6ccccc6)c45)cc3)nc(-c3cccc(-n4c5ccccc5c5ccccc54)c3)n2)cc1]

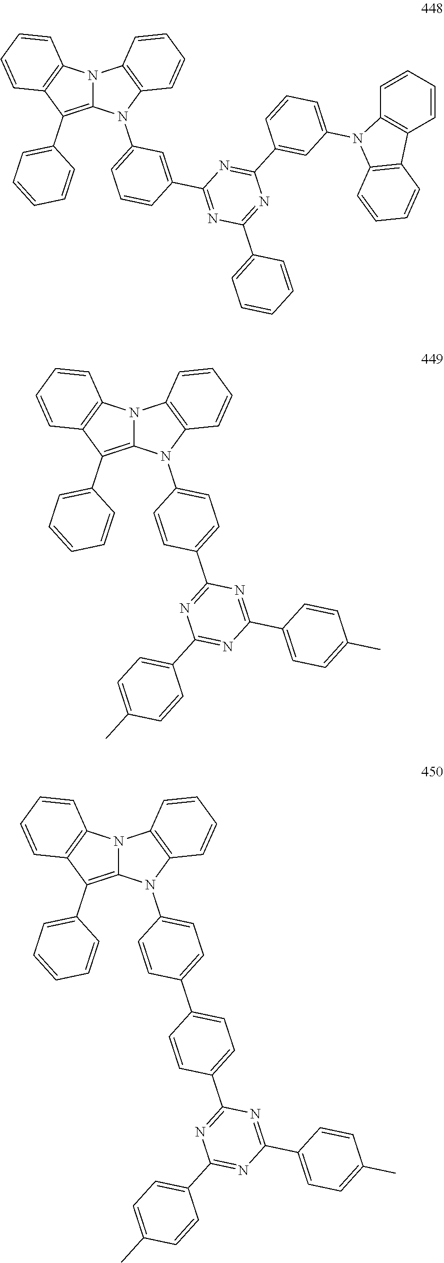 [SMILES: c1ccc(-c2nc(-c3cccc(-n4c5ccccc5c5ccccc54)c3)nc(-c3cccc(-n4c5ccccc5n5c6ccccc6c(-c6ccccc6)c45)c3)n2)cc1 ; Cc1ccc(-c2nc(-c3ccc(C)cc3)nc(-c3ccc(-n4c5ccccc5n5c6ccccc6c(-c6ccccc6)c45)cc3)n2)cc1 ; Cc1ccc(-c2nc(-c3ccc(C)cc3)nc(-c3ccc(-c4ccc(-n5c6ccccc6n6c7ccccc7c(-c7ccccc7)c56)cc4)cc3)n2)cc1]

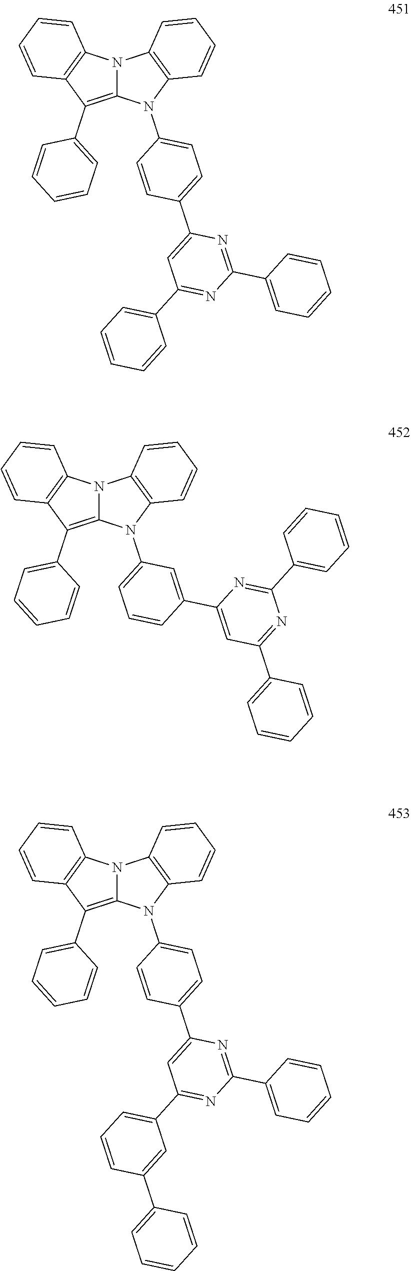 [SMILES: c1ccc(-c2cc(-c3ccc(-n4c5ccccc5n5c6ccccc6c(-c6ccccc6)c45)cc3)nc(-c3ccccc3)n2)cc1 ; c1ccc(-c2cc(-c3cccc(-n4c5ccccc5n5c6ccccc6c(-c6ccccc6)c45)c3)nc(-c3ccccc3)n2)cc1 ; c1ccc(-c2cccc(-c3cc(-c4ccc(-n5c6ccccc6n6c7ccccc7c(-c7ccccc7)c56)cc4)nc(-c4ccccc4)n3)c2)cc1]

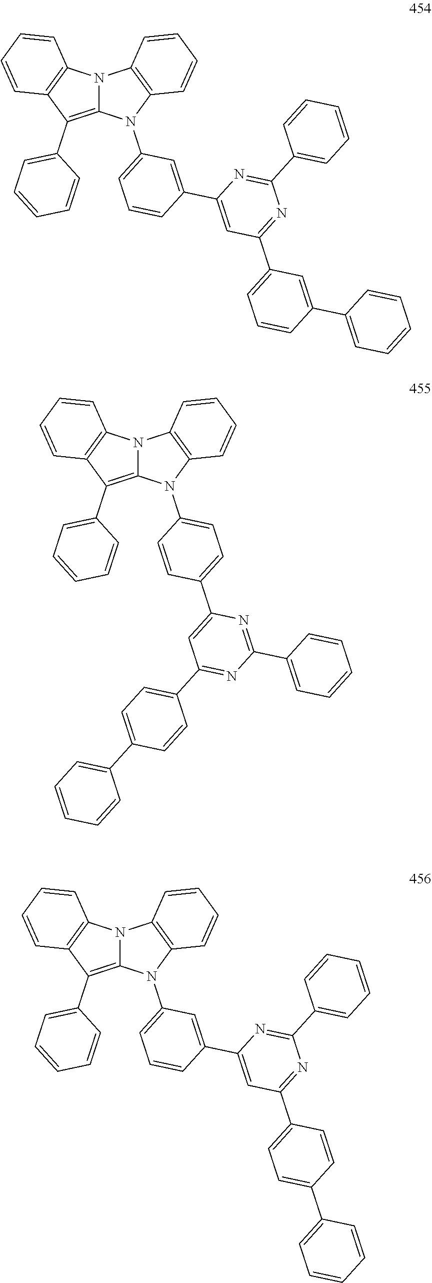 [SMILES: c1ccc(-c2cccc(-c3cc(-c4cccc(-n5c6ccccc6n6c7ccccc7c(-c7ccccc7)c56)c4)nc(-c4ccccc4)n3)c2)cc1 ; c1ccc(-c2ccc(-c3cc(-c4ccc(-n5c6ccccc6n6c7ccccc7c(-c7ccccc7)c56)cc4)nc(-c4ccccc4)n3)cc2)cc1 ; c1ccc(-c2ccc(-c3cc(-c4cccc(-n5c6ccccc6n6c7ccccc7c(-c7ccccc7)c56)c4)nc(-c4ccccc4)n3)cc2)cc1]

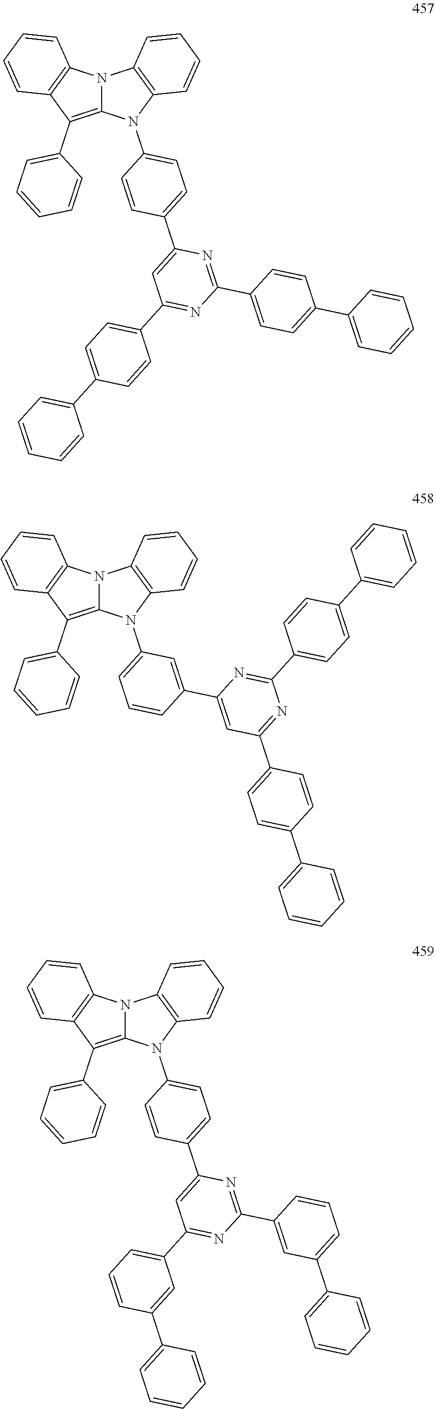 [SMILES: c1ccc(-c2ccc(-c3cc(-c4ccc(-n5c6ccccc6n6c7ccccc7c(-c7ccccc7)c56)cc4)nc(-c4ccc(-c5ccccc5)cc4)n3)cc2)cc1 ; c1ccc(-c2ccc(-c3cc(-c4cccc(-n5c6ccccc6n6c7ccccc7c(-c7ccccc7)c56)c4)nc(-c4ccc(-c5ccccc5)cc4)n3)cc2)cc1 ; c1ccc(-c2cccc(-c3cc(-c4ccc(-n5c6ccccc6n6c7ccccc7c(-c7ccccc7)c56)cc4)nc(-c4cccc(-c5ccccc5)c4)n3)c2)cc1]

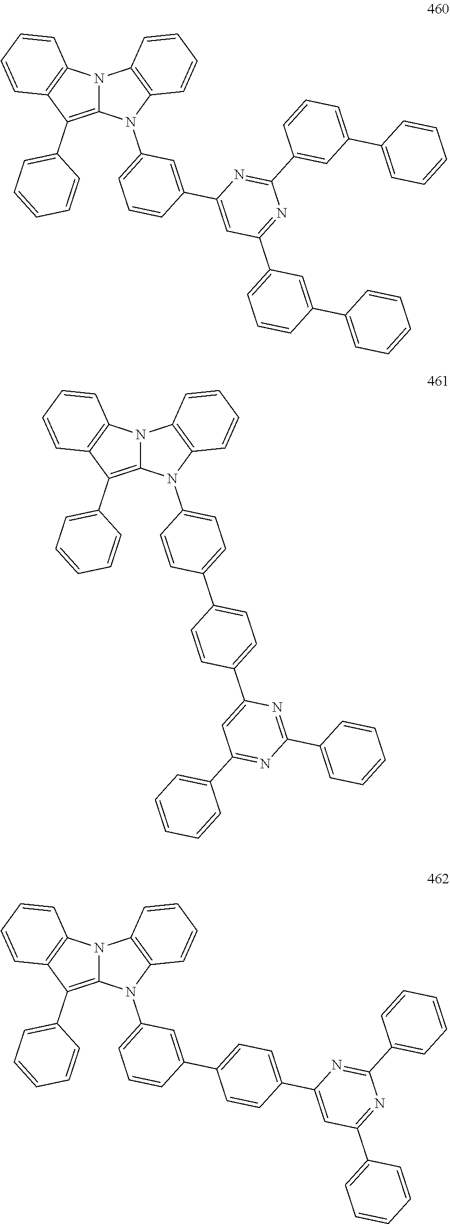 [SMILES: c1ccc(-c2cccc(-c3cc(-c4cccc(-n5c6ccccc6n6c7ccccc7c(-c7ccccc7)c56)c4)nc(-c4cccc(-c5ccccc5)c4)n3)c2)cc1 ; c1ccc(-c2cc(-c3ccc(-c4ccc(-n5c6ccccc6n6c7ccccc7c(-c7ccccc7)c56)cc4)cc3)nc(-c3ccccc3)n2)cc1 ; c1ccc(-c2cc(-c3ccc(-c4cccc(-n5c6ccccc6n6c7ccccc7c(-c7ccccc7)c56)c4)cc3)nc(-c3ccccc3)n2)cc1]

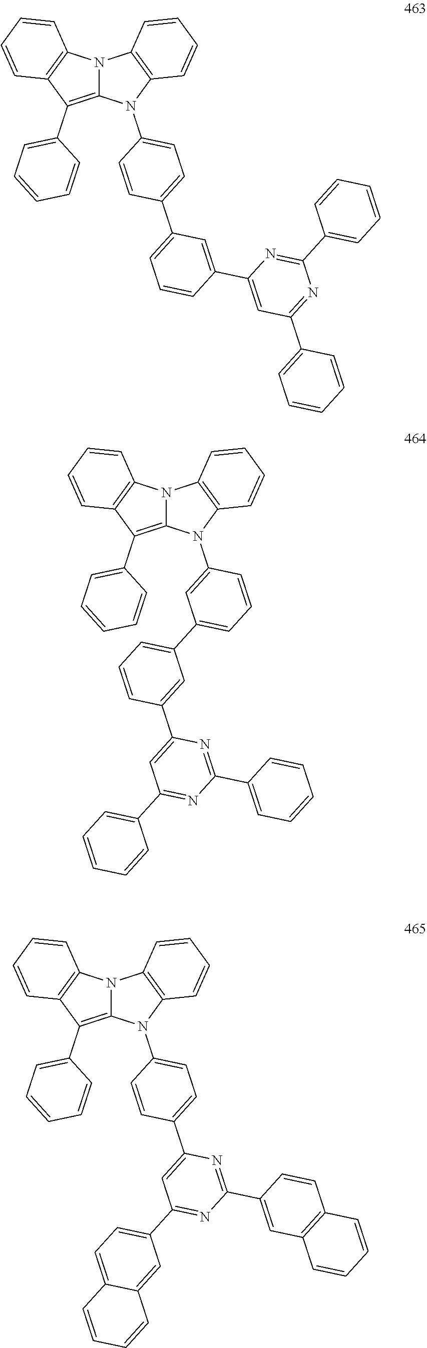 [SMILES: c1ccc(-c2cc(-c3cccc(-c4ccc(-n5c6ccccc6n6c7ccccc7c(-c7ccccc7)c56)cc4)c3)nc(-c3ccccc3)n2)cc1 ; c1ccc(-c2cc(-c3cccc(-c4cccc(-n5c6ccccc6n6c7ccccc7c(-c7ccccc7)c56)c4)c3)nc(-c3ccccc3)n2)cc1 ; c1ccc(-c2c3ccccc3n3c4ccccc4n(-c4ccc(-c5cc(-c6ccc7ccccc7c6)nc(-c6ccc7ccccc7c6)n5)cc4)c23)cc1]

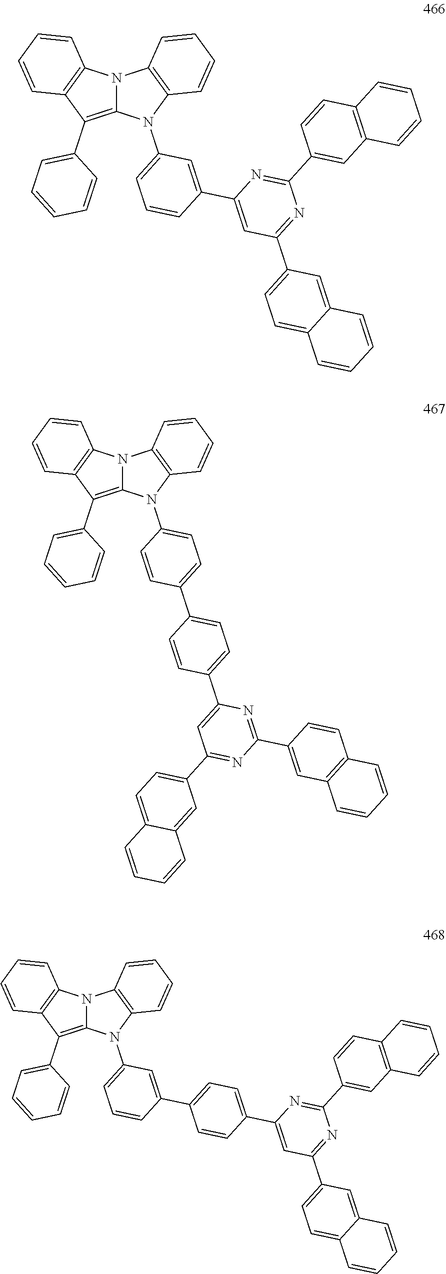 [SMILES: c1ccc(-c2c3ccccc3n3c4ccccc4n(-c4cccc(-c5cc(-c6ccc7ccccc7c6)nc(-c6ccc7ccccc7c6)n5)c4)c23)cc1 ; c1ccc(-c2c3ccccc3n3c4ccccc4n(-c4ccc(-c5ccc(-c6cc(-c7ccc8ccccc8c7)nc(-c7ccc8ccccc8c7)n6)cc5)cc4)c23)cc1 ; c1ccc(-c2c3ccccc3n3c4ccccc4n(-c4cccc(-c5ccc(-c6cc(-c7ccc8ccccc8c7)nc(-c7ccc8ccccc8c7)n6)cc5)c4)c23)cc1]

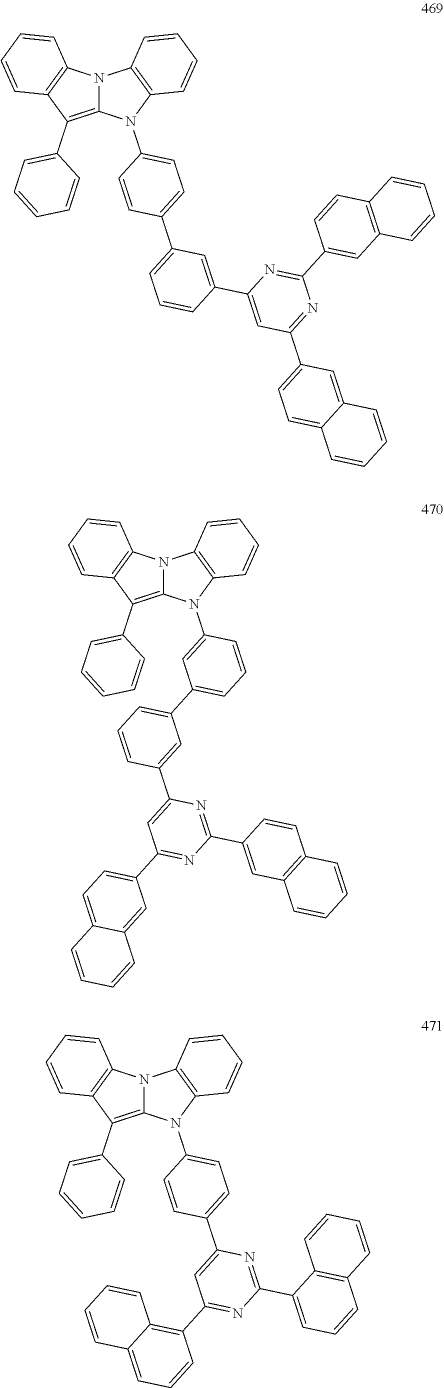 [SMILES: c1ccc(-c2c3ccccc3n3c4ccccc4n(-c4ccc(-c5cccc(-c6cc(-c7ccc8ccccc8c7)nc(-c7ccc8ccccc8c7)n6)c5)cc4)c23)cc1 ; c1ccc(-c2c3ccccc3n3c4ccccc4n(-c4cccc(-c5cccc(-c6cc(-c7ccc8ccccc8c7)nc(-c7ccc8ccccc8c7)n6)c5)c4)c23)cc1 ; c1ccc(-c2c3ccccc3n3c4ccccc4n(-c4ccc(-c5cc(-c6cccc7ccccc67)nc(-c6cccc7ccccc67)n5)cc4)c23)cc1]

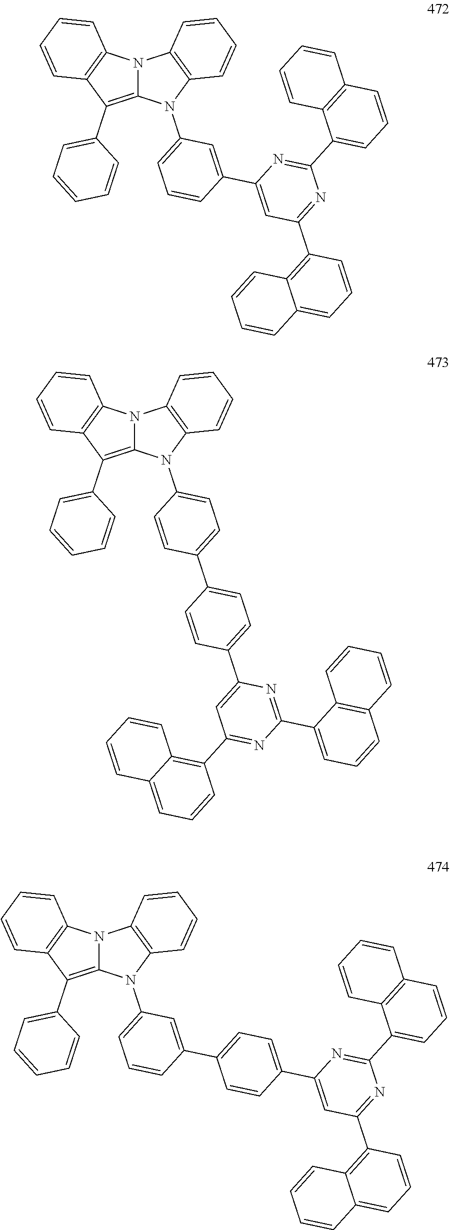 [SMILES: c1ccc(-c2c3ccccc3n3c4ccccc4n(-c4cccc(-c5cc(-c6cccc7ccccc67)nc(-c6cccc7ccccc67)n5)c4)c23)cc1 ; c1ccc(-c2c3ccccc3n3c4ccccc4n(-c4ccc(-c5ccc(-c6cc(-c7cccc8ccccc78)nc(-c7cccc8ccccc78)n6)cc5)cc4)c23)cc1 ; c1ccc(-c2c3ccccc3n3c4ccccc4n(-c4cccc(-c5ccc(-c6cc(-c7cccc8ccccc78)nc(-c7cccc8ccccc78)n6)cc5)c4)c23)cc1]

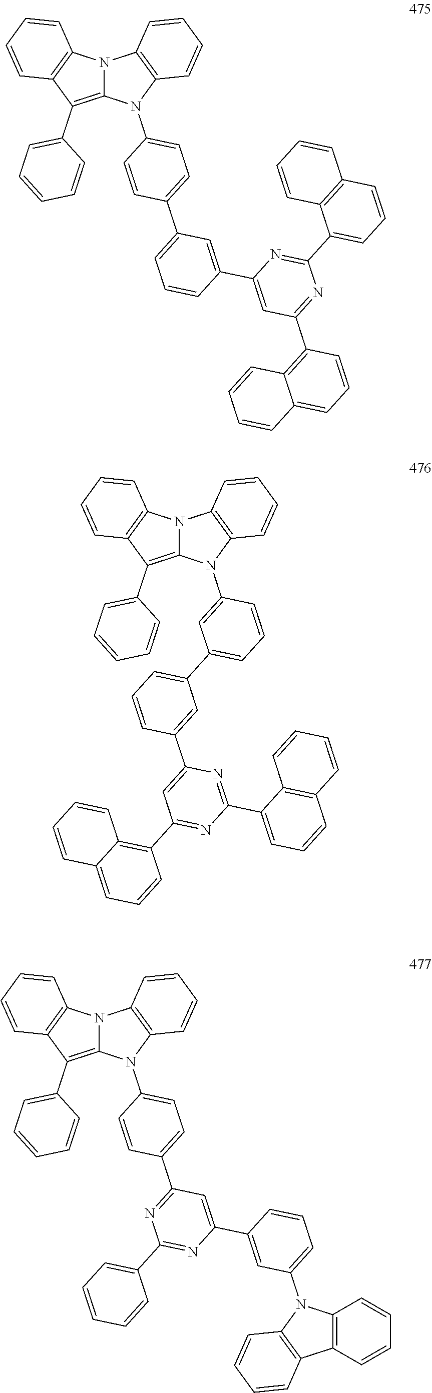 [SMILES: c1ccc(-c2c3ccccc3n3c4ccccc4n(-c4ccc(-c5cccc(-c6cc(-c7cccc8ccccc78)nc(-c7cccc8ccccc78)n6)c5)cc4)c23)cc1 ; c1ccc(-c2c3ccccc3n3c4ccccc4n(-c4cccc(-c5cccc(-c6cc(-c7cccc8ccccc78)nc(-c7cccc8ccccc78)n6)c5)c4)c23)cc1 ; c1ccc(-c2nc(-c3ccc(-n4c5ccccc5n5c6ccccc6c(-c6ccccc6)c45)cc3)cc(-c3cccc(-n4c5ccccc5c5ccccc54)c3)n2)cc1]

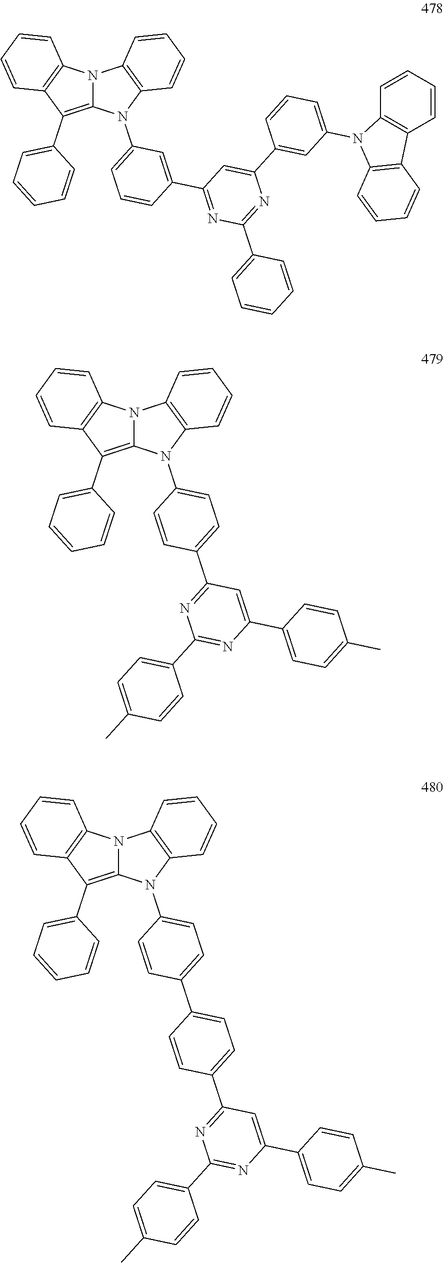 [SMILES: c1ccc(-c2nc(-c3cccc(-n4c5ccccc5c5ccccc54)c3)cc(-c3cccc(-n4c5ccccc5n5c6ccccc6c(-c6ccccc6)c45)c3)n2)cc1 ; Cc1ccc(-c2cc(-c3ccc(-n4c5ccccc5n5c6ccccc6c(-c6ccccc6)c45)cc3)nc(-c3ccc(C)cc3)n2)cc1 ; Cc1ccc(-c2cc(-c3ccc(-c4ccc(-n5c6ccccc6n6c7ccccc7c(-c7ccccc7)c56)cc4)cc3)nc(-c3ccc(C)cc3)n2)cc1]

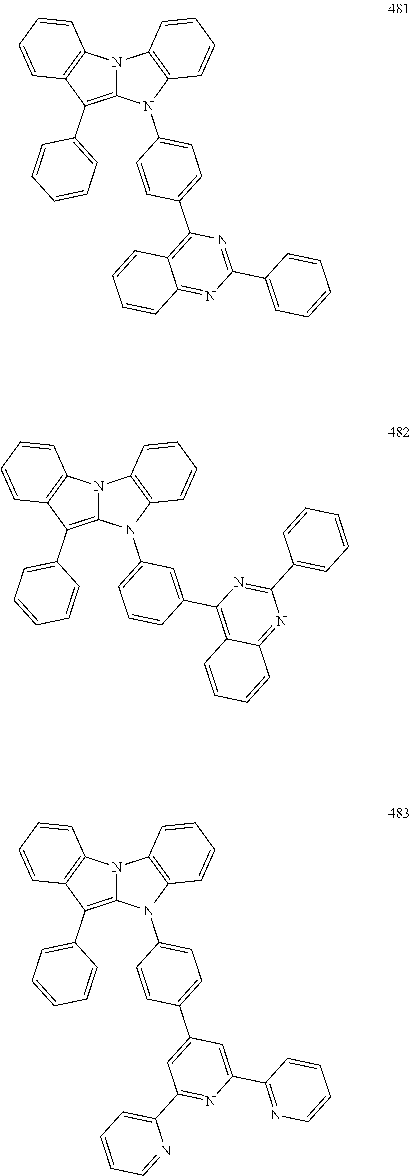 [SMILES: c1ccc(-c2nc(-c3ccc(-n4c5ccccc5n5c6ccccc6c(-c6ccccc6)c45)cc3)c3ccccc3n2)cc1 ; c1ccc(-c2nc(-c3cccc(-n4c5ccccc5n5c6ccccc6c(-c6ccccc6)c45)c3)c3ccccc3n2)cc1 ; c1ccc(-c2c3ccccc3n3c4ccccc4n(-c4ccc(-c5cc(-c6ccccn6)nc(-c6ccccn6)c5)cc4)c23)cc1]

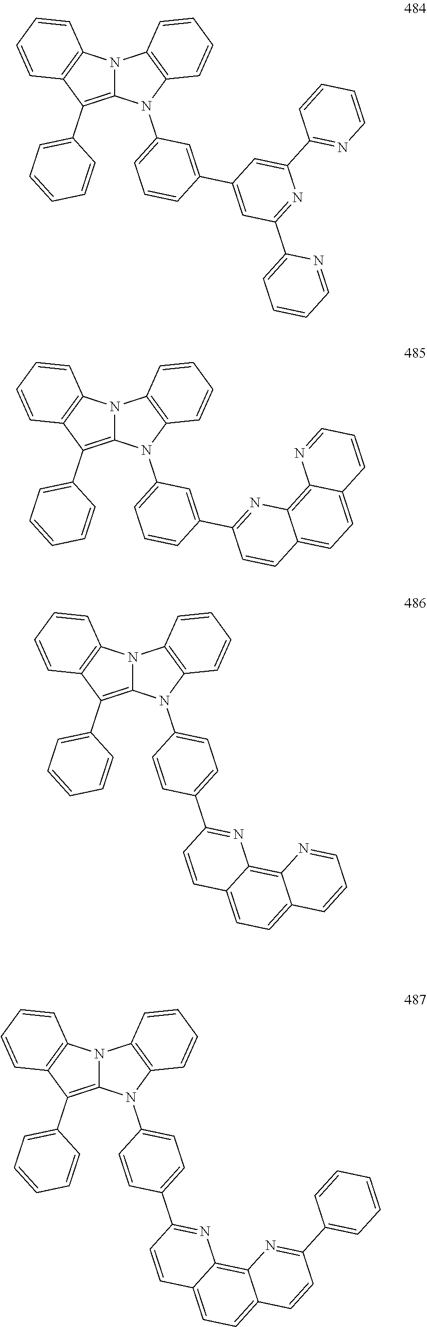 [SMILES: c1ccc(-c2c3ccccc3n3c4ccccc4n(-c4cccc(-c5cc(-c6ccccn6)nc(-c6ccccn6)c5)c4)c23)cc1 ; c1ccc(-c2c3ccccc3n3c4ccccc4n(-c4cccc(-c5ccc6ccc7cccnc7c6n5)c4)c23)cc1 ; c1ccc(-c2c3ccccc3n3c4ccccc4n(-c4ccc(-c5ccc6ccc7cccnc7c6n5)cc4)c23)cc1 ; c1ccc(-c2ccc3ccc4ccc(-c5ccc(-n6c7ccccc7n7c8ccccc8c(-c8ccccc8)c67)cc5)nc4c3n2)cc1]

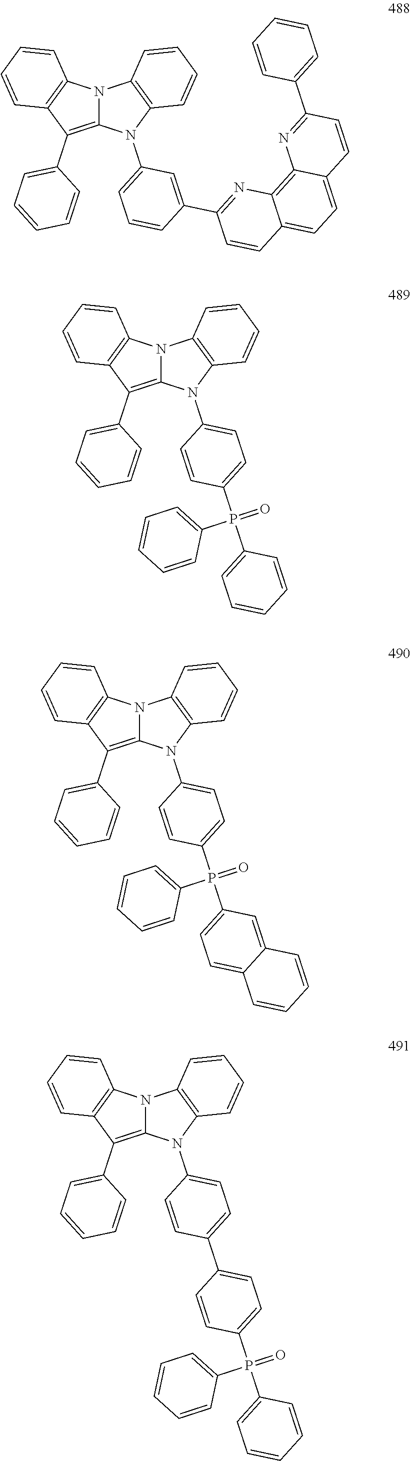 [SMILES: c1ccc(-c2ccc3ccc4ccc(-c5cccc(-n6c7ccccc7n7c8ccccc8c(-c8ccccc8)c67)c5)nc4c3n2)cc1 ; O=P(c1ccccc1)(c1ccccc1)c1ccc(-n2c3ccccc3n3c4ccccc4c(-c4ccccc4)c23)cc1 ; O=P(c1ccccc1)(c1ccc(-n2c3ccccc3n3c4ccccc4c(-c4ccccc4)c23)cc1)c1ccc2ccccc2c1 ; O=P(c1ccccc1)(c1ccccc1)c1ccc(-c2ccc(-n3c4ccccc4n4c5ccccc5c(-c5ccccc5)c34)cc2)cc1]

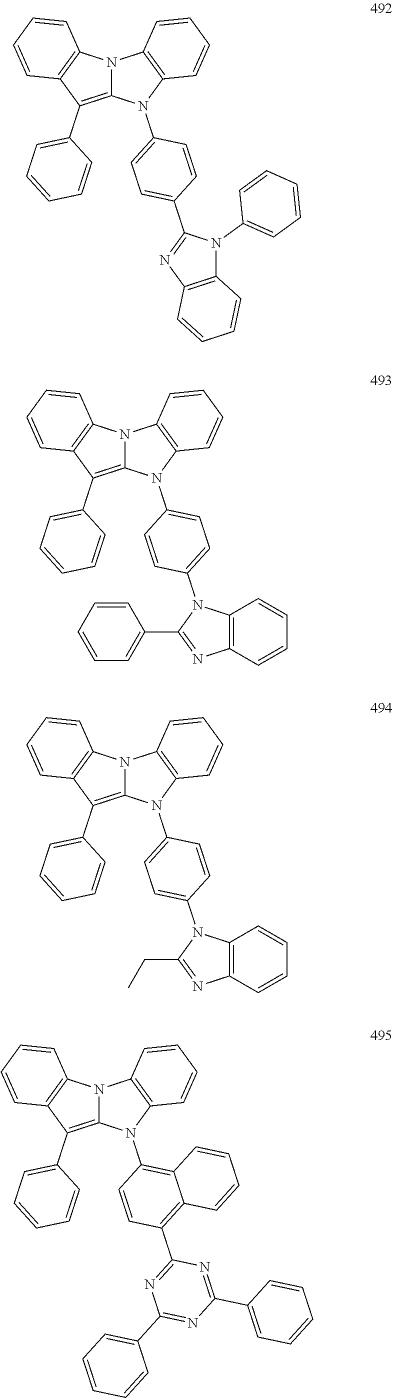 [SMILES: c1ccc(-c2c3ccccc3n3c4ccccc4n(-c4ccc(-c5nc6ccccc6n5-c5ccccc5)cc4)c23)cc1 ; c1ccc(-c2c3ccccc3n3c4ccccc4n(-c4ccc(-n5c(-c6ccccc6)nc6ccccc65)cc4)c23)cc1 ; CCc1nc2ccccc2n1-c1ccc(-n2c3ccccc3n3c4ccccc4c(-c4ccccc4)c23)cc1 ; c1ccc(-c2nc(-c3ccccc3)nc(-c3ccc(-n4c5ccccc5n5c6ccccc6c(-c6ccccc6)c45)c4ccccc34)n2)cc1]

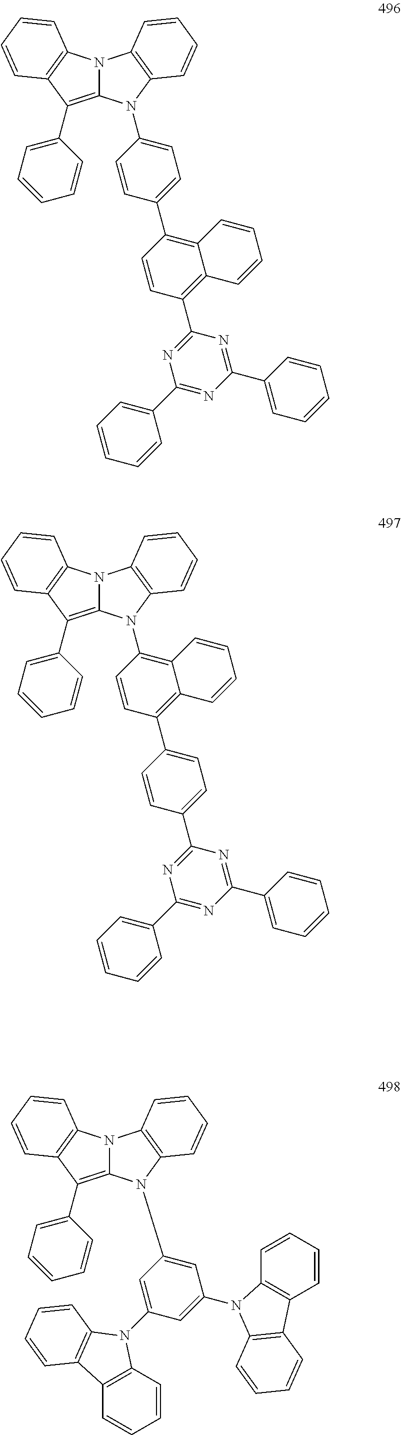 [SMILES: c1ccc(-c2nc(-c3ccccc3)nc(-c3ccc(-c4ccc(-n5c6ccccc6n6c7ccccc7c(-c7ccccc7)c56)cc4)c4ccccc34)n2)cc1 ; c1ccc(-c2nc(-c3ccccc3)nc(-c3ccc(-c4ccc(-n5c6ccccc6n6c7ccccc7c(-c7ccccc7)c56)c5ccccc45)cc3)n2)cc1 ; c1ccc(-c2c3ccccc3n3c4ccccc4n(-c4cc(-n5c6ccccc6c6ccccc65)cc(-n5c6ccccc6c6ccccc65)c4)c23)cc1]

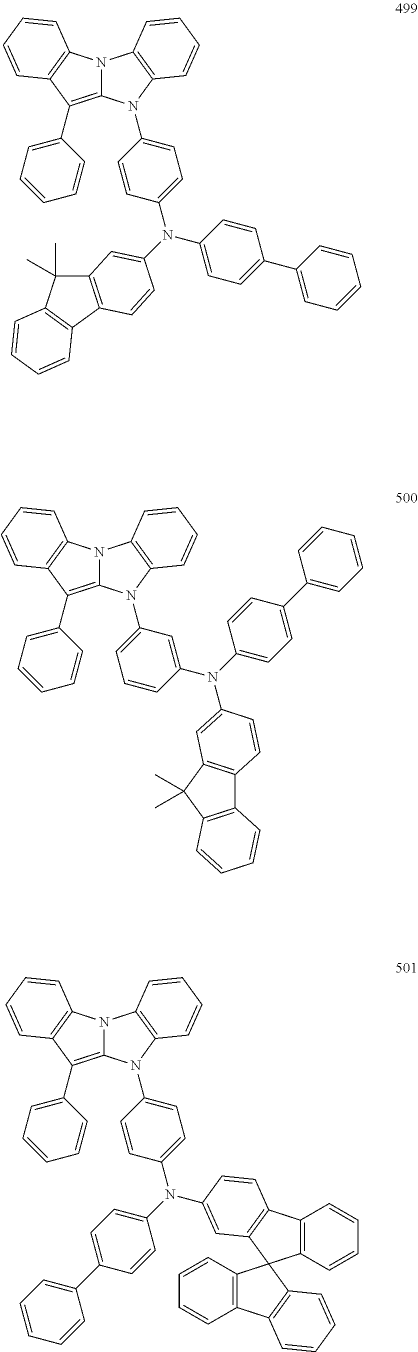 [SMILES: CC1(C)c2ccccc2-c2ccc(N(c3ccc(-c4ccccc4)cc3)c3ccc(-n4c5ccccc5n5c6ccccc6c(-c6ccccc6)c45)cc3)cc21 ; CC1(C)c2ccccc2-c2ccc(N(c3ccc(-c4ccccc4)cc3)c3cccc(-n4c5ccccc5n5c6ccccc6c(-c6ccccc6)c45)c3)cc21 ; c1ccc(-c2ccc(N(c3ccc(-n4c5ccccc5n5c6ccccc6c(-c6ccccc6)c45)cc3)c3ccc4c(c3)C3(c5ccccc5-c5ccccc53)c3ccccc3-4)cc2)cc1]

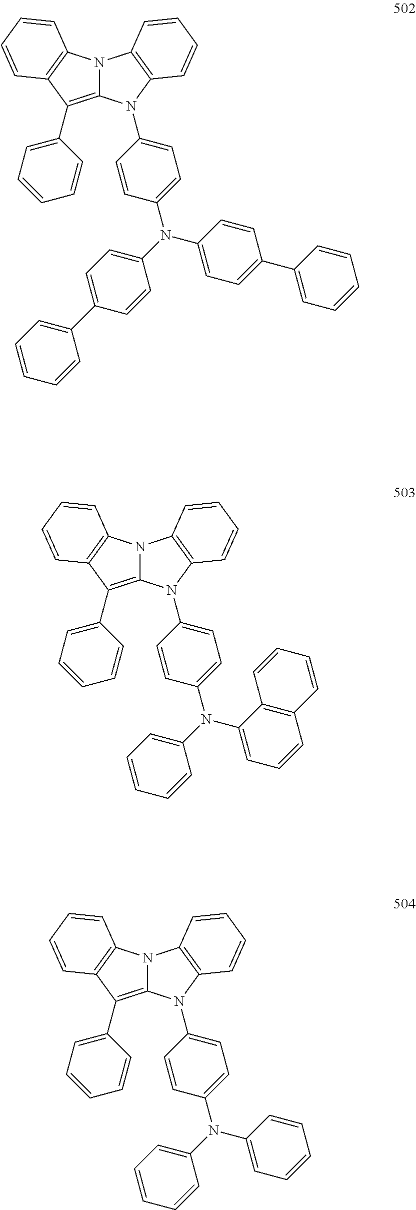 [SMILES: c1ccc(-c2ccc(N(c3ccc(-c4ccccc4)cc3)c3ccc(-n4c5ccccc5n5c6ccccc6c(-c6ccccc6)c45)cc3)cc2)cc1 ; c1ccc(-c2c3ccccc3n3c4ccccc4n(-c4ccc(N(c5ccccc5)c5cccc6ccccc56)cc4)c23)cc1 ; c1ccc(-c2c3ccccc3n3c4ccccc4n(-c4ccc(N(c5ccccc5)c5ccccc5)cc4)c23)cc1]

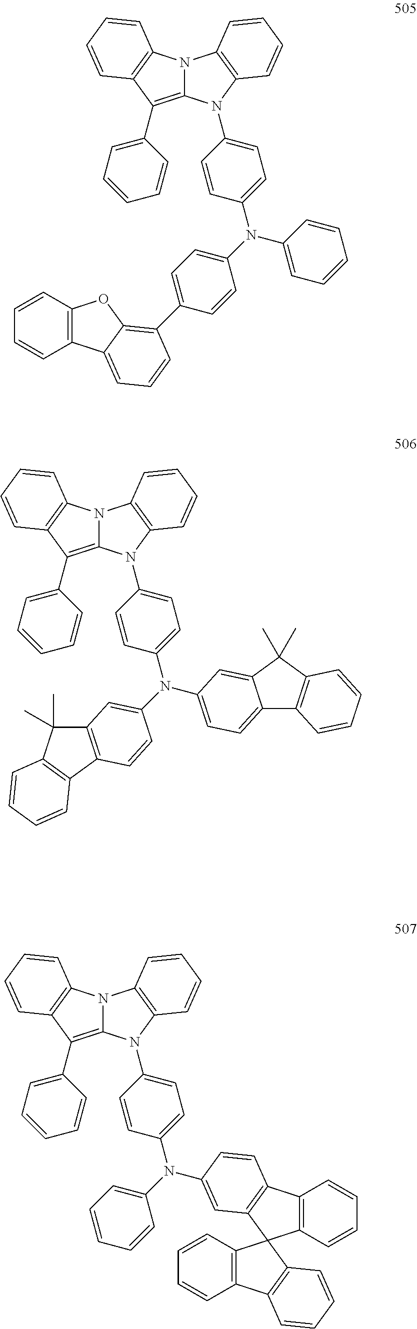 [SMILES: c1ccc(-c2c3ccccc3n3c4ccccc4n(-c4ccc(N(c5ccccc5)c5ccc(-c6cccc7c6oc6ccccc67)cc5)cc4)c23)cc1 ; CC1(C)c2ccccc2-c2ccc(N(c3ccc(-n4c5ccccc5n5c6ccccc6c(-c6ccccc6)c45)cc3)c3ccc4c(c3)C(C)(C)c3ccccc3-4)cc21 ; c1ccc(-c2c3ccccc3n3c4ccccc4n(-c4ccc(N(c5ccccc5)c5ccc6c(c5)C5(c7ccccc7-c7ccccc75)c5ccccc5-6)cc4)c23)cc1]

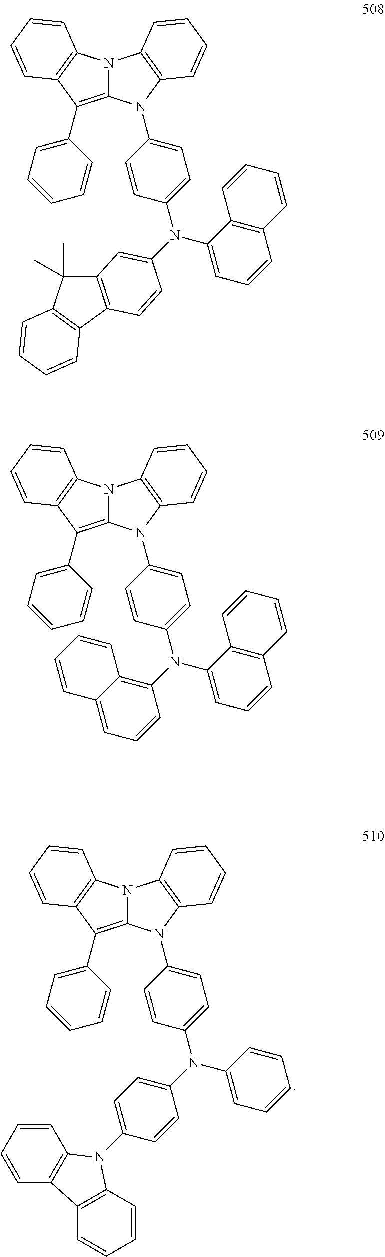 [SMILES: CC1(C)c2ccccc2-c2ccc(N(c3ccc(-n4c5ccccc5n5c6ccccc6c(-c6ccccc6)c45)cc3)c3cccc4ccccc34)cc21 ; c1ccc(-c2c3ccccc3n3c4ccccc4n(-c4ccc(N(c5cccc6ccccc56)c5cccc6ccccc56)cc4)c23)cc1 ; c1ccc(-c2c3ccccc3n3c4ccccc4n(-c4ccc(N(c5ccccc5)c5ccc(-n6c7ccccc7c7ccccc76)cc5)cc4)c23)cc1]

8. An organic light emitting device comprising:
a first electrode;
a second electrode provided opposite to the first electrode; and
one or more organic material layers provided between the first electrode and the second electrode,
wherein one or more layers of the organic material layers comprise the heterocyclic compound of claim 1.

9. The organic light emitting device of claim 8, wherein the organic material layer comprises a hole transfer layer, and the hole transfer layer comprises the heterocyclic compound.

10. The organic light emitting device of claim 8, wherein the organic material layer comprises an electron injection layer or an electron transfer layer, and the electron injection layer or the electron transfer layer comprises the heterocyclic compound.

11. The organic light emitting device of claim 8, wherein the organic material layer comprises an electron blocking layer or a hole blocking layer, and the electron blocking layer or the hole blocking layer comprises the heterocyclic compound.

12. The organic light emitting device of claim 8, further comprising one, two or more layers selected from the group consisting of a light emitting layer, a hole injection layer, a hole transfer layer, an electron injection layer, an electron transfer layer, an electron blocking layer and a hole blocking layer.

13. The organic light emitting device of claim 8 comprising:
a first stack provided on the first electrode and comprising a first light emitting layer;
a charge generation layer provided on the first stack;
a second stack provided on the charge generation layer and comprising a second light emitting layer; and
the second electrode provided on the second stack.

14. The organic light emitting device of claim 13, wherein the charge generation layer comprises the heterocyclic compound.

15. The organic light emitting device of claim 13, wherein the charge generation layer is an N-type charge generation layer, and the charge generation layer comprises the heterocyclic compound.
